(19) United States
(12) Patent Application Publication
(10) Pub. No.: US 2025/0145589 A1
(43) Pub. Date: May 8, 2025

(54) PYRIDINE DERIVATIVE AND USE THEREOF

(71) Applicants: SICHUAN HUIYU PHARMACEUTICAL CO., LTD., Neijiang (CN); SICHUAN HUIYU SEACROSS PHARMACEUTICAL TECHNOLOGY CO., LTD., Chengdu (CN)

(72) Inventors: Shoujun CHEN, Neijiang (CN); Xiaoming QIANG, Neijiang (CN); Zhao DING, Neijiang (CN); Yong XIONG, Neijiang (CN); Haibo WANG, Neijiang (CN); Ke LIU, Neijiang (CN); Mingdeng LIU, Neijiang (CN)

(73) Assignees: SICHUAN HUIYU PHARMACEUTICAL CO., LTD., Neijiang (CN); SICHUAN HUIYU SEACROSS PHARMACEUTICAL TECHNOLOGY CO., LTD., Chengdu (CN)

(21) Appl. No.: 18/692,386

(22) PCT Filed: Sep. 21, 2022
(86) PCT No.: PCT/CN2022/120154

§ 371 Date: Mar. 15, 2024

(30) Foreign Application Priority Data
Sep. 22, 2021 (CN) 202111108176.5
Jan. 25, 2022 (CN) 202210089257.3
Mar. 8, 2022 (CN) 202210227315.4

(51) Int. Cl. C07D 401/14; A61K 31/517; A61K 31/519; A61K 31/55; A61K 45/06; A61P 35/00; C07D 401/04; C07D 409/14; C07D 413/14; C07D 417/14; C07D 471/04; C07D 471/08; C07D 471/10; C07D 487/04; C07D 487/08; C07D 487/10

(52) U.S. Cl. CPC C07D 401/14; A61K 31/517; A61K 31/519; A61K 31/55; A61K 45/06; A61P 35/00; C07D 401/04; C07D 409/14; C07D 413/14; C07D 417/14; C07D 471/04; C07D 471/08; C07D 471/10; C07D 487/04; C07D 487/08; C07D 487/10

(57) ABSTRACT

A pyridine derivative and use thereof are provided. The compound has an obvious inhibitory effect on the activity of KRAS and PI3K proteins, can be used as a KRAS and/or PI3K protein inhibitor, and can be used for preparing a drug for treatment of diseases such as KRAS or PI3K protein-mediated cancer. The compound has an extremely strong inhibitory effect on a KRAS G12C inhibitor (e.g. AMG-510) drug-resistant cancer cells, and has a wide application prospect thereof.

### TECHNICAL FIELD

The present disclosure relates to the field of medical technology, and in particular to a compound used as a KRAS G12C inhibitor and/or a PI3K protein inhibitor and its uses.

### BACKGROUND

RAS protein is an important signaling molecule that participates in various processes such as cell proliferation, differentiation, survival and movement. The RAS family consists of KRAS. NRAS, and HRAS. They have two states in cells: an inactive state bound to GDP and an activated state bound to GTP. When RAS is activated, it can activate multiple downstream signaling pathways, including the MAPK signaling pathway, PI3K signaling pathway, and Ral-GEFs signaling pathway. These signaling pathways play an important role in promoting cell survival, proliferation, and cytokine release.

Activation of RAS relies on receptor phosphorylation after tyrosine kinase receptor binding to signaling molecules, exposing the binding site. Guanine Nucleotide Exchange Factor (GEF, such as SOS1) and growth factor receptor binding protein (Grb2) complex Binds to this site and binds to it through the SH2 domain of Grb2 to form an RTK/Grb2/SOS complex. SHP2 can further promote the binding of this complex to RAS, prompting it to release GDP and bind GTP, thereby activating RAS. In addition, it is worth noting that before activation, RAS needs to be bound to the inner side of the cell membrane to exert its physiological effects, in which farnesyl transferase plays a key role, and sphingomyelin synthase-1 may also be involved in regulating the localization of RAS. As the proto-oncogene with the highest mutation rate, the RAS family consists of KRAS, NRAS and HRAS respectively. 84% of patients have KRAS mutations, while only 4% and 12% have HRAS and NRAS mutations respectively. The KRAS (Kirsten Rat Sarcoma virus) gene is located on autosome 12, consists of 188-189 amino acids, and has a molecular weight of 21.7 kDa. RAS protein consists of 6 β-sheets and 5 α-helices, of which the 166 amino acids at the N-terminus constitute the G domain, while the C-terminus is the membrane-binding region.

The G domain (divided into G1-G5) is a functional domain that can directly bind to GDP/GTP, G2 (Switch I) binds to GTP, while G3 (Switch II) binds to GDP. KRAS is widely expressed in body tissues, and its mRNA levels can be detected in almost all tissues. KRAS functions in cells by participating in GTP hydrolysis. As a GTPase, it functions in catalyzing the conversion of guanine triphosphate (GTP) and guanine diphosphate (GDP), promoting Cell survival, migration and proliferation. In normal cells, KRAS mainly binds to GDP and is in an inactive state.

However, Guanine Nucleotide Exchange Factor (GEF) such as SOS1 can promote the release of GDP and combine with GTP to become an activated state. The reverse transformation can be achieved through GTPase-activating protein (GAP), such as RasGAP. KRAS accounts for the majority of RAS mutations. KRAS mutations occur widely in cancer patients, including 5-30% of lung cancer patients, 36-40% of colon cancer patients, and about 90% of pancreatic cancer patients. In addition, KRAS mutations have also been found in patients with other tumors, such as endometrial cancer, skin cancer, and multiple myeloma.

The G12 mutations are the most common among KRAS mutations, accounting for 83%, followed by G13, which accounts for 14%, and Q61, G12V, G12D, and G12C are the most common among the G12 mutations, G12C mutations account for 14% of patients with non-small cell lung cancer, 5% of colon cancer, and 2% of pancreatic ductal adenocarcinoma. Mutations in KRAS can promote its binding to GTP and remain in an activated state, continuously activating cell growth, leading to tumor occurrence. All these indicate that KRAS mutations have important value in clinical treatment, G12C mutation is a relatively common subtype of KRAS gene mutation, which refers to the mutation of glycine 12 to cysteine. KRAS G12C mutations are also the most common in lung cancer. According to data reported in the literature (Nat Rev Drug Discov 2014; 13:828-851), KRAS G12C mutations account for about 10% of all lung cancer patients. For more than 30 years, there has been no breakthrough in the search for drugs that specifically target KRAS, so KRAS is generally considered an “Undruggable Target” protein target.

In recent years, the druggability of KRAS G12C has been discovered, and KRAS G12C inhibitors have become one of the current popular areas of drug research and development. The literature (Nature. 2013; 503: 548-551) reports a class of covalent binding inhibitors targeting the KRAS G12C mutation. However, these compounds have low enzymatic activity and do not show activity at the cellular level. Another type of compound reported in the literature (Science 2016; 351: 604-608. Cancer Discov 2016; 6: 316-29) shows u M-level cellular anti-proliferative activity at the cellular level, but its metabolic stability is poor and its activity is difficult to improve further. Finding drugs that target RAS is very difficult. Because the binding ability of GTP and RAS is very strong, it is difficult to find small molecules that can competitively inhibit their binding; moreover, the surface of RAS protein is very smooth and lacks structural space for small molecules or drugs to bind. In 2013, the University of California discovered through protein crystallography research that a common mutant KRAS G12C protein among KRAS forms a new pocket on the surface of the molecule after binding to GDP. Small molecule inhibitors can co-operate with the KRAS G12C protein at this site. Valent binding locks the protein in an inactive state. In recent years, AraxesPharma has applied for several patents targeting KRAS G12C inhibitors. For example, WO2016164675 and WO2016168540 reported a class of quinazoline derivatives with high enzyme-binding activity and demonstrated μM-level cell anti-proliferative activity. Its structure is stable and has certain selectivity. Amgen (WO2018119183) and AstraZeneca (WO2018206539A1) respectively disclosed patents on KRAS G12C inhibitors in 2018, and Amgen's KRASG12C inhibitor AMG-510 launched a phase I clinical study in July 2018.

Looking at the KRAS G12C inhibitors currently reported in the literature, they all have an acrylamide fragment, which acts as a Michael addition receptor and a cysteine residue on the KRAS G12C mutant protein to form a covalent binding complex. In 2018, Liu Yi et al. published a report on Cell (Matthew, R. Janes, Yi Liu, et al., Cell, 2018, 172, 578-589.), a covalent binding inhibitor ARS-1620 targeting the KRAS G12C mutation. The compound has good metabolic stability, exhibits nM-level cellular anti-proliferative activity at the cellular level, and can effectively inhibit tumor growth in the pancreatic cancer MIA-Paca2 cell subcutaneous xenograft tumor model. Currently, the KRAS G12C inhibitors under development that are progressing rapidly include Araxes' ARS-1620, Amgen's AMG-510, and Mirati's MRTX-849 (WO2020216190A1). Among them. AMG-510 is making the fastest progress and was approved for marketing in 2021. It started phase I clinical trials in 2018 and is the first KRAS G12C inhibitor to enter clinical trials.

However, resistance to KRAS inhibitors is a major problem in cancer treatment. Amgen's latest clinical trial results also show that some patients treated with the KRAS G12C inhibitor AMG-510 continued to progress after remission. Piro Lito et al. discovered rapid non-uniform adaptation to conformation-specific KRAS G12C inhibition. Because KRAS G12C cycles between active and inactive conformations, and inhibitors bind only to the latter, the researchers tested whether syngeneic cell populations responded in a non-uniform manner by studying the effects of treatment at single-cell resolution. The researchers found that shortly after treatment, some cancer cells were rendered quiescent and had lower KRAS activity, while others bypassed this effect to resume proliferation and develop drug resistance (XIE, J. Y.; et al, Nature 2020, 577, 421-425). The latest research by Adachi and others shows that epithelial-mesenchymal transition (EMT) is the cause of intrinsic and acquired resistance to AMG510 (Adachi, Y., et al Clin Cancer Res 2020, Sep. 8. doi: 10.1158/1078-0432.CCR-20-2077. Online ahead of print.). Although there are other studies published on the resistance mechanism of AMG-510, there is still no good practical solution to the problem of resistance to KRAS inhibitors such as AMG-510 in clinical practice. Therefore, it is of great significance to develop new therapeutic agents that can overcome the drug resistance problem.

### SUMMARY

The major technical problem solved by the present disclosure is to provide a pyridine derivative, which, as a selective inhibitor of KRAS mutation, has the advantages of high activity, good selectivity and low toxic and side effects. At the same time, it also has a good inhibitory effect on mutation sites that cause resistance to other KRAS inhibitors. In addition, it also shows a strong inhibitory effect on PI3K.

In order to solve the above technical problems, the present disclosure provides a compound of formula I″″, or a tautomer, meso isomer, racemate, enantiomer, diastereomer or mixture thereof, or a pharmaceutically acceptable hydrate, solvate or salt thereof:

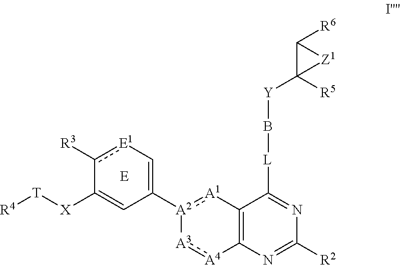

Wherein,
T is selected from S(O)₂, C═O, CH₂ or NHS(O)₂;
X is selected from NR¹, S or S(O)₂;
A¹ is selected from CH, CH₂, C═O or N, A² is selected from C, CH or N, A³ is selected from CR⁸, CR⁹R¹⁰ or N, A⁴ is selected from CR²⁹, CR⁹R¹⁰ or N;
Z¹ is a bond or O;
E1 is NR¹¹, CH or CH₂;
B is

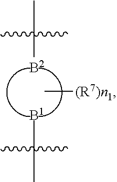

the ring where B¹, B² are located is a 5- to 10-membered nitrogen-containing heterocycloalkyl or a 5- to 10-membered nitrogen-containing heterocycloalkenyl, wherein the cycloalkyl or cycloalkenyl is a monocyclic ring, spirocyclic ring or bridged cyclic ring, B¹ is selected from CR¹² or N, B² is selected from CR¹³ or N, and at least one of B¹ and B² is N;
or, B is

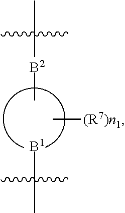

the ring where B¹ is located is a 4- to 8-membered nitrogen-containing monoheterocycloalkyl, B¹ is selected from CR¹² or N, B² is selected from CR¹³R^{13′} or NR¹⁴;
or, B is

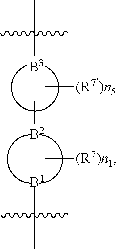

the ring where B¹, B² are located is a 5- to 10-membered nitrogen-containing heterocycloalkyl and is a monocyclic ring, spirocyclic ring or bridged cyclic ring, B¹ is selected from CR¹² or N, B² is selected from CR^{13′} or N, and at least one of B¹ and B² is N; the ring where B³ is located is a 4- to 10-membered nitrogen-containing heterocycloalkyl and is a monocyclic ring, spirocyclic ring or bridged cyclic ring, B³ is selected from CR^{12′} or N;
B¹ is connected to L;
L is selected from a bond or NR¹⁵;
Y is selected from C═O or S(O)₂;
R⁵, R⁶ are independently selected from hydrogen, halogen, cyano, alkyl, aliphatic heterocyclyl, aryl, heteroaryl, —OR^{a}, —NR^{b}R^{c}, —C(O)R¹⁶, —S(O)R¹⁶, —S(O)₂R¹⁶, —P(O)R¹⁶R¹⁷, —C(O)NR^{b}R^{c} or —C(O)OR^{a}, wherein the alkyl, aliphatic heterocyclyl, aryl, heteroaryl are optionally substituted by one or more R¹⁸;
each occurrence of R¹⁸ is independently selected from halogen, cyano, alkyl, cycloalkyl, aliphatic heterocyclyl, —OR^{a}, —NR^{b}R^{c}, —C(O)R¹⁶, —C(O)NR^{b}R^{c} or —C(O)OR^{a};
R¹, R¹¹, R¹⁴, R¹⁵ are independently selected from hydrogen, alkyl, cycloalkyl, aliphatic heterocyclyl or —C(O)R¹⁹, wherein the alkyl, cycloalkyl, aliphatic heterocyclyl are optionally substituted by one or more substituents selected from the group consisting of: halogen, cyano, hydroxy, amino, alkyl, cycloalkyl and aliphatic heterocyclyl;
n₁, n₅ are independently selected from 0, 1, 2, 3 or 4;
each occurrence of R¹², R^{12′}, R¹³, R^{13′}, R^{13″}, R⁷, R^{7′} is independently selected from hydrogen, halogen, cyano, alkyl, cycloalkyl, aliphatic heterocyclyl, aryl, heteroaryl, —ORⁿ, —NR^{o}R^{p}, —C(O)R²⁰, —C(O)NR^{o}R^{p} or —C(O)ORⁿ, wherein the alkyl, cycloalkyl, aliphatic heterocyclyl, aryl, heteroaryl are optionally substituted by one or more R²¹;
each occurrence of R²¹ is independently selected from halogen, cyano, alkyl, cycloalkyl, aliphatic heterocyclyl, —ORⁿ, —NR^{o}R^{p}, —C(O)R²⁰, —C(O)NR^{o}R^{p} or —C(O)ORⁿ;
or Z¹ is a bond, R⁵, R⁶ together with the atoms to which R⁵, R⁶ are attached form a 4- to 9-membered cycloalkenyl, or R⁵, R¹⁴ together with the fragment to which R⁵, R¹⁴ are attached form a 4- to 9-membered aliphatic heterocyclyl, or R⁶, R¹³ together with the fragment to which R⁶, R¹³ are attached form a 4- to 9-membered cycloalkenyl, or R⁵, R¹³ together with the fragment to which R⁵, R¹³ are attached form a 4- to 9-membered alicyclyl, or R⁶, R¹⁴ together with the fragment to which R⁶, R¹⁴ are attached form a 4- to 9-membered aliphatic heterocyclyl, or R⁵, R⁶ together with the atoms to which R⁵, R⁶ are attached form a 6- to 10-membered aryl or 5- to 10-membered heteroaryl, wherein the cycloalkenyl, alicyclic heterocyclyl, alicyclyl, aryl and heteroaryl are optionally substituted by one or more substituents selected from the group consisting of: halogen, cyano, hydroxy, amino, alkyl, cycloalkyl and alicyclic heterocyclyl;
R² is selected from hydrogen, halogen, cyano, alkyl, cycloalkyl, alicyclic heterocyclyl, aryl, heteroaryl, —OR^{d}, —NR^{e}R^{f}, —C(O)R²², —C(O)R^{e}R^{f} or —C(O)OR^{d}, wherein the alkyl, cycloalkyl, alicyclic heterocyclyl, aryl, heteroaryl are optionally substituted by one or more R²²;
each occurrence of R²³ is independently selected from halogen, cyano, alkyl, cycloalkyl, aliphatic heterocyclyl, —OR^{d}, —NR^{e}R^{f}, —C(O)R²², —C(O)NR^{e}R^{f} or —C(O)OR^{d};
R³ is selected from hydrogen, halogen, cyano, alkyl, cycloalkyl, aliphatic heterocyclyl, aryl, heteroaryl, —OR^{g}, —SR^{g}, —NR^{h}Rⁱ, —C(O)R²⁴, —C(O)R^{h}Rⁱ or —C(O)OR^{g}, when the ‘ [symbol] ’ in ring E is absent, R³ can also be a carbonyl group that together formed by the connected carbon atom, wherein the alkyl, cycloalkyl, aliphatic heterocyclyl, aryl, heteroaryl are optionally substituted by one or more R²⁵;
each occurrence of R²⁵ is independently selected from halogen, cyano, alkyl, cycloalkyl, aliphatic heterocyclyl, —OR^{g}, —NR^{h}Rⁱ, —C(O)R²⁴, —C(O)NR^{h}Rⁱ or —C(O)OR^{g};
R⁴ is selected from alkyl, cycloalkyl, aliphatic heterocyclyl, aryl or heteroaryl, wherein the alkyl, cycloalkyl, aliphatic heterocyclyl, aryl, heteroaryl are optionally substituted by one or more R²⁶;
each occurrence of R²⁶ is independently selected from halogen, cyano, alkyl, cycloalkyl, aliphatic heterocyclyl, aryl, heteroaryl, —OR^{j}, —NR^{k}R^{m}, —C(O)R²⁷, —C(O)NR^{k}R^{m} or —C(O)OR^{j}, wherein the alkyl, cycloalkyl, aliphatic heterocyclyl, aryl, heteroaryl are optionally substituted by one or more R²⁸;
each occurrence of R²⁸ is independently selected from halogen, cyano, alkyl, cycloalkyl, aliphatic heterocyclyl, —OR^{j}, —NR^{k}R^{m}, —C(O)R²⁷, —C(O)NR^{k}R^{m} or —C(O)OR^{j};
each occurrence of R⁸, R⁹, R¹⁰, R²⁹ is independently selected from hydrogen, halogen, cyano, alkyl, cycloalkyl, aliphatic heterocyclyl, aryl, heteroaryl, —OR^{q}, —NR^{r}R^{s}, —C(O)R³⁰, —C(O)NR^{r}R^{s} or —C(O)OR^{q}; wherein the alkyl, cycloalkyl, aliphatic heterocyclyl, aryl, heteroaryl are optionally substituted by one or more R³¹;
When the ‘ [symbol] ’ in ring E is a bond, R¹¹ is absent;
each occurrence of R³⁰ independently selected from halogen, cyano, alkyl, cycloalkyl, aliphatic heterocyclyl, —OR^{q}, —NR^{r}R^{s}, —C(O)R³⁰, —C(O)NR^{r}R^{s} or —C(O)OR^{q};
each occurrence of R^{a}, R^{b}, R^{c}, R^{d}, R^{e}, R^{f}, R^{g}, R^{h}, Rⁱ, R^{j}, R^{k}, R^{m}, Rⁿ, R^{o}, R^{p}, R^{q}, R^{r}, R^{s} is independently selected from H, alkyl, cycloalkyl, aliphatic heterocyclyl or —C(O)R³²; wherein the alkyl, cycloalkyl, alicyclic heterocyclyl are optionally substituted by one or more substituents selected from the group consisting of: halogen, cyano, hydroxy amino, alkyl, cycloalkyl and alkyl substituted or unsubstituted aliphatic heterocyclyl;
each occurrence of R¹⁶, R¹⁷, R¹⁹, R²⁰, R²², R²⁴, R²⁷, R³⁰, R³² is independently selected from H, alkyl, cycloalkyl or alicyclic heterocyclyl, the alkyl, cycloalkyl, alicyclic heterocyclyl are optionally substituted by one or more of the following substituents: halogen, cyano, hydroxy, amino, alkyl, cycloalkyl, aliphatic heterocyclyl.

In some embodiments of the present disclosure, the compound of the present disclosure has the structure shown in formula I′″, or a tautomer, meso isomer, racemate, enantiomer, diastereomer or mixture thereof, or a pharmaceutically acceptable hydrate, solvate or salt thereof:

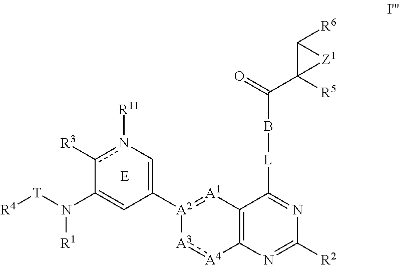

wherein,
T is selected from S(O)₂, C═O, CH₂ or NHS(O)₂;
A¹ is selected from CH, CH₂, C═O or N, A² is selected from C, CH or N, A³ is selected from CR⁸, CR⁹R¹⁰ or N, A⁴ is selected from CR²⁹, CR⁹R¹⁰ or N;
Z¹ is a bond or O;
B is

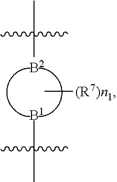

the ring where B¹, B² are located is a 5- to 10-membered nitrogen-containing heterocycloalkyl and is a monocyclic ring, spirocyclic ring or bridged cyclic ring, B¹ is selected from CR¹² or N, B² is selected from CR¹³ or N, and at least one of B¹ and B² is N;
or, B is

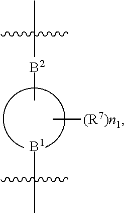

the ring where B¹ is located is a 4- to 8-membered nitrogen-containing monoheterocycloalkyl, B¹ is selected from CR¹² or N, B² is selected from CR¹³R^{13′} or NR¹⁴;
or, B is

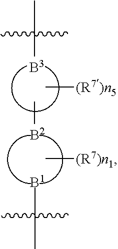

the ring where B¹, B² are located is a 5- to 10-membered nitrogen-containing heterocycloalkyl and is a monocyclic ring, spirocyclic ring or bridged cyclic ring, B¹ is selected from CR¹² or N, B² is selected from CR^{13″} or N, and at least one of B¹ and B² is N; the ring where B³ is located is a 4- to 10-membered nitrogen-containing heterocycloalkyl and is a monocyclic ring, spirocyclic ring or bridged cyclic ring, B³ is selected from CR^{12′} or N;
B¹ is connected to L;
L is selected from a bond or NR;
R⁵, R⁶ are independently selected from hydrogen, halogen, cyano, alkyl, aliphatic heterocyclyl, aryl, heteroaryl, —OR^{a}, —NR^{b}R^{c}, —C(O)R¹⁶, —S(O)R¹⁶, —S(O)₂R¹⁶, —P(O)R¹⁶R¹⁷, —C(O)NR^{b}R^{c} or —C(O)OR^{a}, wherein the alkyl, aliphatic heterocyclyl, aryl, heteroaryl are optionally substituted by one or more R¹⁸;
each occurrence of R¹⁸ is independently selected from halogen, cyano, alkyl, cycloalkyl, aliphatic heterocyclyl, —OR^{a}, —NR^{b}R^{c}, —C(O)R¹⁶, —C(O)NR^{b}R^{c} or —C(O)OR^{a};
R¹, R¹¹, R¹⁴, R¹⁵ are independently selected from hydrogen, alkyl, cycloalkyl, aliphatic heterocyclyl or —C(O)R¹⁹, wherein the alkyl, cycloalkyl, aliphatic heterocyclyl are optionally substituted by one or more substituents selected from the group consisting of: halogen, cyano, hydroxy, amino, alkyl, cycloalkyl and aliphatic heterocyclyl;
n₁, n₅ are independently selected from 0, 1, 2, 3 or 4;
each occurrence of R¹², R^{12′}, R¹³, R^{13′}, R^{13″}, R⁷, R^{7′} is independently selected from hydrogen, halogen, cyano, alkyl, cycloalkyl, aliphatic heterocyclyl, aryl, heteroaryl, —ORⁿ, —NR^{o}R^{p}, —C(O)R²⁰, —C(O)NR^{o}R^{p} or —C(O)ORⁿ, wherein the alkyl, cycloalkyl, aliphatic heterocyclyl, aryl, heteroaryl are optionally substituted by one or more R²¹;
each occurrence of R²¹ is independently selected from halogen, cyano, alkyl, cycloalkyl, aliphatic heterocyclyl, —ORⁿ, —NR^{o}R^{p}, —C(O)R²⁰, —C(O)NR^{o}R^{p} or —C(O)ORⁿ;
or Z¹ is a bond, R⁵, R⁶ together with the atoms to which R⁵, R⁶ are attached form a 4- to 9-membered cycloalkenyl, or R⁵, R¹⁴ together with the fragment to which R⁵, R¹⁴ are attached form a 4- to 9-membered aliphatic heterocyclyl, or R⁶, R¹³ together with the fragment to which R⁶, R¹³ are attached form a 4- to 9-membered cycloalkenyl, or R⁵, R¹³ together with the fragment to which R⁵, R¹³ are attached form a 4- to 9-membered alicyclyl, or R⁶, R¹⁴ together with the fragment to which R⁶, R¹⁴ are attached form a 4- to 9-membered aliphatic heterocyclyl, or R⁵, R⁶ together with the atoms to which R⁵, R⁶ are attached form a 6- to 10-membered aryl or 5- to 10-membered heteroaryl, wherein the cycloalkenyl, alicyclic heterocyclyl, alicyclyl, aryl and heteroaryl are optionally substituted by one or more substituents selected from the group consisting of: halogen, cyano, hydroxy, amino, alkyl, cycloalkyl and alicyclic heterocyclyl;
R² is selected from hydrogen, halogen, cyano, alkyl, cycloalkyl, alicyclic heterocyclyl, aryl, heteroaryl, —OR⁴, —NR^{e}R^{f}, —C(O)R²², —C(O)R^{e}R^{f} or —C(O)OR^{d}, wherein the alkyl, cycloalkyl, alicyclic heterocyclyl, aryl, heteroaryl are optionally substituted by one or more R²³;
each occurrence of R²³ is independently selected from halogen, cyano, alkyl, cycloalkyl, aliphatic heterocyclyl, —OR^{d}, —NR^{e}R^{f}, —C(O)R²², —C(O)NR^{e}R^{f} or —C(O)OR^{d};
R³ is selected from hydrogen, halogen, cyano, alkyl, cycloalkyl, aliphatic heterocyclyl, aryl, heteroaryl, —OR^{g}, —NR^{h}Rⁱ, —C(O)R²⁴, —C(O)R^{h}Rⁱ or —C(O)OR^{g}, when the ‘ [symbol] ’ in ring E is absent, R³ can also be the carbonyl group that together formed by the connected carbon atom, wherein the alkyl, cycloalkyl, aliphatic heterocyclyl, aryl, heteroaryl are optionally substituted by one or more R²⁵;
each occurrence of R²⁵ is independently selected from halogen, cyano, alkyl, cycloalkyl, aliphatic heterocyclyl, —OR^{g}, —NR^{h}Rⁱ, —C(O)R²⁴, —C(O)NR^{h}Rⁱ or —C(O)OR^{g};
R⁴ is selected from alkyl, cycloalkyl, aliphatic heterocyclyl, aryl or heteroaryl, wherein the alkyl, cycloalkyl, aliphatic heterocyclyl, aryl, heteroaryl are optionally substituted by one or more R²⁶;
each occurrence of R²⁶ is independently selected from halogen, cyano, alkyl, cycloalkyl, aliphatic heterocyclyl, aryl, heteroaryl, —OR^{j}, —NR^{k}R^{m}, —C(O)R²⁷, —C(O)NR^{k}R^{m} or —C(O)OR^{j}, wherein the alkyl, cycloalkyl, aliphatic heterocyclyl, aryl, heteroaryl are optionally substituted by one or more R²⁸;
each occurrence of R²⁸ is independently selected from halogen, cyano, alkyl, cycloalkyl, aliphatic heterocyclyl, —OR^{j}, —NR^{k}R^{m}, —C(O)R²⁷, —C(O)NR^{k}R^{m} or —C(O)OR^{j};
each occurrence of R⁸, R⁹, R¹⁶, R²⁹ is independently selected from hydrogen, halogen, cyano, alkyl, cycloalkyl, aliphatic heterocyclyl, aryl, heteroaryl, —OR^{q}, —NR^{r}R^{s}, —C(O)R³⁰, —C(O)NR^{r}R^{s} or —C(O)OR^{q}; wherein the alkyl, cycloalkyl, aliphatic heterocyclyl, aryl, heteroaryl are optionally substituted by one or more R³¹;
When the ‘ [symbol] ’ in ring E is a bond, R¹¹ is absent;
each occurrence R³¹ is independently selected from halogen, cyano, alkyl, cycloalkyl, aliphatic heterocyclyl, —OR^{q}, —NR^{r}R^{s}, —C(O)R³, —C(O)NR^{r}R^{s} or —C(O)OR^{q};
each occurrence of R^{a}, R^{b}, R^{c}, R^{d}, R^{e}, R^{f}, R^{g}, R^{h}, Rⁱ, R^{j}, R^{k}, R^{m}, Rⁿ, R^{o}, R^{p}, R^{q}, R^{r}, R^{s} is independently selected from H, alkyl, cycloalkyl, aliphatic heterocyclyl or —C(O)R³²; wherein the alkyl, cycloalkyl, alicyclic heterocyclyl are optionally substituted by one or more substituents selected from the group consisting of: halogen, cyano, hydroxy, amino, alkyl, cycloalkyl and aliphatic heterocyclyl;
each occurrence of R¹⁶, R¹⁷, R¹⁹, R²⁰, R²², R²⁴, R²⁷, R³⁰, R³¹ is independently selected from H, alkyl, cycloalkyl or alicyclic heterocyclyl, wherein the alkyl, cycloalkyl, alicyclic heterocyclyl are optionally substituted by one or more substituents selected from the group consisting of: halogen, cyano, hydroxy, amino, alkyl, cycloalkyl and aliphatic heterocyclyl.

In some embodiments of the present disclosure, the compound of the present disclosure has the structure shown in formula I′, or a tautomer, meso isomer, racemate, enantiomer, diastereomer or mixture thereof, or a pharmaceutically acceptable hydrate, solvate or salt thereof:

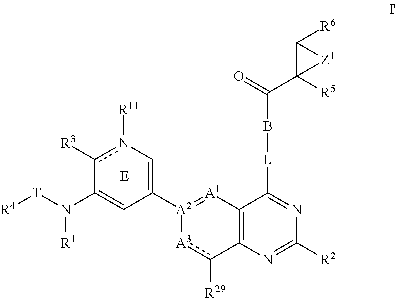

Wherein,
T is selected from S(O)₂, C═O, CH₂ or NHS(O)₂;
A¹ is selected from CH, CH₂, C═O or N, A² is selected from C or N, A³ is selected from CR⁸, CR⁹R¹⁰ or N;
Z¹ is a bond or O;
B is

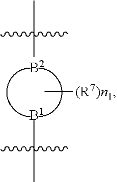

the ring where B¹, B² are located is a 5- to 10-membered nitrogen-containing heterocycloalkyl and is a monocyclic ring, spirocyclic ring or bridged cyclic ring, B¹ is selected from CR¹² or N, B² is selected from CR¹³ or N, and at least one of B¹ and B² is N;
or, B is

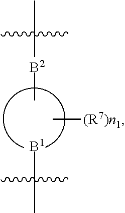

the ring where B¹ is located is a 4- to 8-membered nitrogen-containing monoheterocycloalkyl, B is selected from CR¹² or N, B² is selected from CR¹³R^{13′} or NR¹⁴;
or, B is

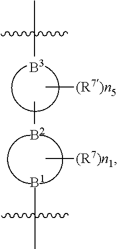

the ring where B¹, B² are located is a 5- to 10-membered nitrogen-containing heterocycloalkyl and is a monocyclic ring, spirocyclic ring or bridged cyclic ring. B¹ is selected from CR¹² or N, B² is selected from CR^{13″} or N, and at least one of B¹ and B² is N; the ring where B³ is located is a 4- to 10-membered nitrogen-containing heterocycloalkyl and is a monocyclic ring, spirocyclic ring or bridged cyclic ring, B³ is selected from CR^{12′} or N;
B¹ is connected to L;
L is selected from a bond or NR¹⁵;
R⁵, R⁶ are independently selected from hydrogen, halogen, cyano, alkyl, aliphatic heterocyclyl, aryl, heteroaryl, —OR^{a}, —NR^{b}R^{c}, —C(O)R¹⁶, —S(O)R¹⁶, —S(O)₂R¹⁶, —P(O)R¹⁶R¹⁷, —C(O)NR^{b}R^{c} or —C(O)OR^{a}, wherein the alkyl, aliphatic heterocyclyl, aryl, heteroaryl are optionally substituted by one or more R¹⁸;
each occurrence of R¹⁸ is independently selected from halogen, cyano, alkyl, cycloalkyl, aliphatic heterocyclyl, —OR, —NR^{b}R^{c}, —C(O)R¹⁶, —C(O)NR^{b}R^{c} or —C(O)OR^{a};
R¹, R¹¹, R¹⁴, R¹⁵ are independently selected from hydrogen, alkyl, cycloalkyl, aliphatic heterocyclyl or —C(O)R¹⁹, wherein the alkyl, cycloalkyl, aliphatic heterocyclyl are optionally substituted by one or more substituents selected from the group consisting of: halogen, cyano, hydroxy, amino, alkyl, cycloalkyl and aliphatic heterocyclyl;
n₁, n₅ are independently selected from 0, 1, 2, 3 or 4;
each occurrence of R¹², R^{12′}, R¹³, R^{13′}, R^{13″}, R⁷, R^{7′} is independently selected from hydrogen, halogen, cyano, alkyl, cycloalkyl, aliphatic heterocyclyl, aryl, heteroaryl, —ORⁿ, —NR^{o}R^{p}, —C(O)R²⁰, —C(O)NR^{o}R^{p} or —C(O)ORⁿ, wherein the alkyl, cycloalkyl, aliphatic heterocyclyl, aryl, heteroaryl are optionally substituted by one or more R²¹;
each occurrence of R²¹ is independently selected from halogen, cyano, alkyl, cycloalkyl, aliphatic heterocyclyl, —ORⁿ, —NR^{o}R^{p}, —C(O)R²⁰, —C(O)NR^{o}R^{p} or —C(O)ORⁿ;
or Z¹ is a bond, R⁵, R⁶ together with the atoms to which R⁵, R⁶ are attached form a 4- to 9-membered cycloalkenyl, or R⁵, R¹⁴ together with the fragment to which R⁵, R¹⁴ are attached form a 4- to 9-membered aliphatic heterocyclyl, or R⁶, R¹³ together with the fragment to which R⁶, R¹³ are attached form a 4- to 9-membered cycloalkenyl, or R⁵, R¹³ together with the fragment to which R⁵, R¹³ are attached form a 4- to 9-membered alicyclyl, or R⁶, R¹⁴ together with the fragment to which R⁵, R¹⁴ are attached form a 4- to 9-membered aliphatic heterocyclyl, or R⁵, R⁶ together with the atoms to which R⁵, R⁶ are attached form a 6- to 10-membered aryl or 5- to 10-membered heteroaryl, wherein the cycloalkenyl, alicyclic heterocyclyl, alicyclyl, aryl and heteroaryl are optionally substituted by one or more substituents selected from the group consisting of: halogen, cyano, hydroxy, amino, alkyl, cycloalkyl and alicyclic heterocyclyl;
R² is selected from hydrogen, halogen, cyano, alkyl, cycloalkyl, alicyclic heterocyclyl, aryl, heteroaryl, —OR^{g}, —NR^{e}R^{f}, —C(O)R²², —C(O)R^{e}R^{f} or —C(O)OR^{d}, wherein the alkyl, cycloalkyl, alicyclic heterocyclyl, aryl, heteroaryl are optionally substituted by one or more R²³;
each occurrence of R²³ is independently selected from halogen, cyano, alkyl, cycloalkyl, aliphatic heterocyclyl, —OR^{d}, —NR^{e}R^{f}, —C(O)R²², —C(O)NR^{e}R^{f} or —C(O)OR^{d};
R³ is selected from hydrogen, halogen, cyano, alkyl, cycloalkyl, aliphatic heterocycyl, aryl, heteroaryl, —OR^{g}, —NR^{h}Rⁱ, —C(O)R²⁴, —C(O)R^{h}Rⁱ or —C(O)OR^{g}, when the ‘ [symbol] ’ in ring E is absent, R³ can also be a carbonyl group that together formed by the connected carbon atom, wherein the alkyl, cycloalkyl, aliphatic heterocyclyl, aryl, heteroaryl are optionally substituted by one or more R²⁵;
each occurrence of R²⁵ is independently selected from halogen, cyano, alkyl, cycloalkyl, aliphatic heterocyclyl, —OR^{g}, —NR^{h}Rⁱ, —C(O)R²⁴, —C(O)NR^{h}Rⁱ or —C(O)OR^{g};
R⁴ is selected from alkyl, cycloalkyl, aliphatic heterocyclyl, aryl or heteroaryl, wherein the alkyl, cycloalkyl, aliphatic heterocyclyl, aryl, heteroaryl are optionally substituted by one or more R²⁶;
each occurrence of R²⁶ is independently selected from halogen, cyano, alkyl, cycloalkyl, aliphatic heterocyclyl, aryl, heteroaryl, —OR^{j}, —NR^{k}R^{m}, —C(O)R²⁷, —C(O)NR^{k}R^{m} or —C(O)OR^{j}, wherein the alkyl, cycloalkyl, aliphatic heterocyclyl, aryl, heteroaryl are optionally substituted by one or more R²⁸;
each occurrence of R²⁸ is independently selected from halogen, cyano, alkyl, cycloalkyl, aliphatic heterocyclyl, —OR^{j}, —NR^{k}R^{m}, —C(O)R²⁷, —C(O)NR^{k}R^{m} or —C(O)OR^{j};
each occurrence of R⁸, R⁹, R¹⁰, R²⁹ is independently selected from hydrogen, halogen, cyano, alkyl, cycloalkyl, aliphatic heterocyclyl, aryl, heteroaryl, —OR^{q}, —NR^{r}R^{s}, —C(O)R³⁰, —C(O)NR^{r}R^{s} or —C(O)OR^{q}; wherein the alkyl, cycloalkyl, aliphatic heterocyclyl, aryl, heteroaryl are optionally substituted by one or more R;
When the ‘ [symbol] ’ in ring E is a bond, R¹¹ is absent;
each occurrence of R³¹ is independently selected from halogen, cyano, alkyl, cycloalkyl, aliphatic heterocyclyl, —OR^{q}, —NR^{r}R^{s}, —C(O)R³⁰, —C(O)NR^{r}R^{s} or —C(O)OR^{q};
each occurrence of R^{a}, R^{b}, R^{c}, R^{d}, R^{e}, R^{f}, R^{g}, R^{h}, Rⁱ, R^{j}, R^{k}, R^{m}, Rⁿ, R^{o}, R^{p}, R^{q}, R^{r}, R^{s} is independently selected from H, alkyl, cycloalkyl, aliphatic heterocyclyl or —C(O)R³²; wherein the alkyl, cycloalkyl, alicyclic heterocyclyl are optionally substituted by one or more substituents selected from the group consisting of: halogen, cyano, hydroxy, amino, alkyl, cycloalkyl and aliphatic heterocyclyl
each occurrence of R¹⁶, R¹⁷, R¹⁹, R²⁰, R²², R²⁴, R²⁷, R³⁰, R³² is independently selected from H, alkyl, cycloalkyl or alicyclic heterocyclyl, the alkyl, cycloalkyl, alicyclic heterocyclyl are optionally substituted by one or more substituents selected from the group consisting of: halogen, cyano, hydroxy, amino, alkyl, cycloalkyl and aliphatic heterocyclyl.

In some embodiments of the present disclosure, the compound of the present disclosure has the structure shown in formula I or a tautomer, meso isomer, racemate, enantiomer, diastereomer or mixture thereof, or a pharmaceutically acceptable hydrate, solvate or salt thereof:

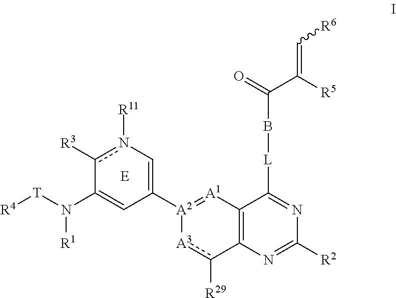

wherein.
T is selected from S(O)₂ or C═O;
A¹ is selected from CH, CH₂, C═O or N, A² is selected from C or N, A³ is selected from CR⁸, CR⁹R¹⁰ or N;
B is

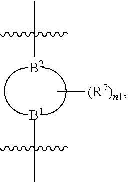

the ring where B¹, B² are located is a 5- to 10-membered nitrogen-containing heterocycloalkyl and is a monocyclic ring, spirocyclic ring or bridged cyclic ring, B¹ is selected from CR¹² or N, B² is selected from CR¹³ or N and at least one of B¹ and B² is N;
or, B is

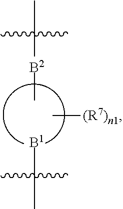

the ring where B¹ is located is a 4- to 8-membered nitrogen-containing monoheterocycloalkyl, B¹ is selected from CR¹² or N, B² is selected from CR¹³R^{13′} or NR¹⁴;
L is selected from a bond or NR¹⁵;
R⁵, R⁶ are independently selected from hydrogen, halogen, cyano, alkyl, aliphatic heterocyclyl, aryl, heteroaryl, —OR^{a}, —NR^{b}R^{c}, —C(O)R¹⁶, —S(O)R¹⁶, —S(O)₂R¹⁶, —P(O)R¹⁶R¹⁷, —C(O)NR^{b}R^{c} or —C(O)OR¹⁸, wherein the alkyl, aliphatic heterocyclyl, aryl, heteroaryl are optionally substituted by one or more R¹⁸;
each occurrence of R¹⁸ is independently selected from halogen, cyano, alkyl, cycloalkyl, aliphatic heterocyclyl, —OR^{a}, —NR^{b}R^{c}, —C(O)R¹⁶, —C(O)NR^{b}R^{c} or —C(O)OR^{a};
R¹, R¹¹, R¹⁴, R¹⁵ are independently selected from hydrogen, alkyl, cycloalkyl, aliphatic heterocyclyl or —C(O)R¹⁹, wherein the alkyl, cycloalkyl, aliphatic heterocyclyl are optionally substituted by one or more of the following substituents: halogen, cyano, hydroxy, amino, alkyl, cycloalkyl, aliphatic heterocyclyl; When the ‘ [symbol] ’ in ring E is a bond, R¹¹ is absent;
n₁ is selected from 0, 1, 2, 3, or 4;
each occurrence of R¹², R¹³, R^{13′}, R⁷ is independently selected from hydrogen, halogen, cyano, alkyl, cycloalkyl, aliphatic heterocyclyl, aryl, heteroaryl, —ORⁿ, —NR^{o}R^{p}, —C(O)R²⁰, —C(O)NR^{o}R^{p} or —C(O)ORⁿ, wherein the alkyl, cycloalkyl, aliphatic heterocyclyl, aryl, heteroaryl are optionally substituted by one or more R²¹;
each occurrence of R²¹ is independently selected from halogen, cyano, alkyl, cycloalkyl, aliphatic heterocyclyl, —ORⁿ, —NR^{o}R^{p}, —C(O)R²⁰, —C(O)NR^{o}R^{p} or —C(O)ORⁿ;
or R⁵, R⁶ together with the atoms to which R⁵, R⁶ are attached form a 4- to 9-membered cycloalkenyl, or R⁵, R¹⁴ together with the fragment to which R⁵, R¹⁴ are attached form a 4- to 9-membered aliphatic heterocyclyl, or R⁶, R¹³ together with the fragment to which R⁶, R¹³ are attached form a 4- to 9-membered cycloalkenyl, or R⁵, R¹³ together with the fragment to which R⁶, R¹⁴ are attached form a 4- to 9-membered alicyclyl, or R⁶, R¹⁴ together with the fragment to which R, R¹⁴ are attached form a 4- to 9-membered aliphatic heterocyclyl,
R² is selected from hydrogen, halogen, cyano, alkyl, cycloalkyl, alicyclic heterocyclyl, aryl, heteroaryl, —OR^{d}, —NR^{e}R^{f}, —C(O)R²¹, —C(O)R^{e}R^{f} or —C(O)OR^{d}, wherein the alkyl, cycloalkyl, alicyclic heterocyclyl, aryl, heteroaryl are optionally substituted by one or more R²³;
each occurrence of R²³ is independently selected from halogen, cyano, alkyl, cycloalkyl, aliphatic heterocyclyl, —OR^{d}, —NR^{e}R^{f}, —C(O)R²², —C(O)NR^{e}R^{f} or —C(O)OR^{d};
R³ is selected from hydrogen, halogen, cyano, alkyl, cycloalkyl, aliphatic heterocyclyl, aryl, heteroaryl, —OR^{g}, —NR^{h}Rⁱ, —C(O)R²⁴, —C(O)R^{h}Rⁱ or —C(O)OR^{g}, when the ‘ [symbol] ’ in ring E is absent, R³ can also be a carbonyl group that together formed by the connected carbon atom, wherein the cycloalkyl, aliphatic heterocyclyl, aryl, heteroaryl are optionally substituted by one or more R²⁵;
each occurrence of R²⁵ is independently selected from halogen, cyano, alkyl, cycloalkyl, aliphatic heterocyclyl, —OR^{g}, —NR^{h}Rⁱ, —C(O)R²⁴, —C(O)NR^{h}Rⁱ or —C(O)OR^{g};
R⁴ is selected from alkyl, cycloalkyl, aliphatic heterocyclyl or aryl, heteroaryl, wherein the alkyl, cycloalkyl, aliphatic heterocyclyl, aryl, heteroaryl are optionally substituted by one or more R²⁶;
each occurrence of R²⁶ is independently selected from halogen, cyano, alkyl, cycloalkyl, aliphatic heterocyclyl, aryl, heteroaryl, —OR^{j}, —NR^{k}R^{m}, —C(O)R^{2′}, —C(O)NR^{k}R^{j} or —C(O)OR^{j}, wherein the alkyl, cycloalkyl, aliphatic heterocyclyl, aryl, heteroaryl are optionally substituted by one or more RV;
each occurrence of R² is independently selected from halogen, cyano, alkyl, cycloalkyl, aliphatic heterocyclyl, —OR^{j}, —NR^{k}R^{m}, —C(O)R²⁷, —C(O)NR^{k}R^{m} or —C(O)OR^{j};
each occurrence of R⁸, R⁹, R¹⁰, R²⁹ is independently selected from hydrogen, halogen, cyano, alkyl, cycloalkyl, aliphatic heterocyclyl, aryl, heteroaryl, —OR^{q}, —NR^{r}R^{s}, —C(O)R³⁰, —C(O)NR^{r}R^{s} or —C(O)OR^{q}; wherein the alkyl, cycloalkyl, aliphatic heterocyclyl, aryl, heteroaryl are optionally substituted by one or more R³¹;
each occurrence of R³¹ is independently selected from halogen, cyano, alkyl, cycloalkyl, aliphatic heterocyclyl, —OR^{q}, —NR^{r}R^{s}, —C(O)R³⁰, —C(O)NR^{r}R^{s} or —C(O)OR^{q};
each occurrence of R^{a}, R^{b}, R^{c}, R^{d}, R^{e}, R^{f}, R^{g}, R^{h}, Rⁱ, R^{j}, R^{k}, R^{m}, Rⁿ, R^{o}, R^{p}, R^{q}, R^{r}, R^{s} is independently selected from H, alkyl, cycloalkyl, aliphatic heterocyclyl or —C(O)R³²; wherein the alkyl, cycloalkyl, alicyclic heterocyclyl are optionally substituted by one or more of the following substituents: halogen, cyano, hydroxy, amino, alkyl, cycloalkyl, aliphatic heterocyclyl;
each occurrence of R¹⁶, R¹⁷, R¹⁹, R²⁰, R²², R²⁴, R²⁷, R³⁰, R³² is independently selected from H, alkyl, cycloalkyl or alicyclic heterocyclyl, the alkyl, cycloalkyl, alicyclic heterocyclyl are optionally substituted by one or more substituents selected from the group consisting of: halogen, cyano, hydroxy, amino, alkyl, cycloalkyl and aliphatic heterocyclyl,
Z¹ is a bond, which means that the two carbon atoms between R⁵ and R⁶ are connected by a double bond:

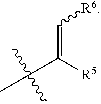

‘Each occurrence of R¹² is independently selected from’ means that when the number of R¹² is greater than 1, different R¹² can be selected from the same or different groups. For example, when the number of R¹² is 2, one R¹² can be selected from alkyl and the other R¹² can be selected from halogen; or, both R¹² can be selected from alkyl; the same applies to other similar situations.

In

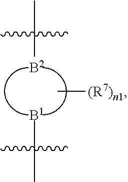

the connection point of R⁷ is not fixed. It should be understood as that R⁷ can be any substitutable position on ring B, and the same applies to other similar situations; but at here, since the substituents on B¹ and B² are limited to R¹² or R¹³, then it should be understood as that the substitutable positions for R⁷ do not include B¹ or B².

The connecting bond of R⁶ is [symbol] , which means that the configuration of Ron the alkenyl is not fixed and can be Z-configuration or E-configuration. The same applies to other similar situations. ‘R⁵, R⁶ together with the atoms to which R⁵, R⁶ are attached form a 4- to 9-membered cycloalkenyl’ means that R⁵, R⁶ and their commonly connected alkenyl fragment together form a 4- to 9-membered cycloalkenyl, that is,

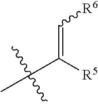

constitutes

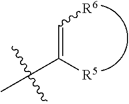

as a 4- to 9-membered cycloalkenyl. The same applies to other similar situations. ‘R⁵, R⁶ together with the atoms to which R⁵, R⁶ are attached form a 6- to 10-membered aryl or 5- to 10-membered heteraryl’ means that R⁵, R⁶ and their commonly connected alkenyl fragment together form a 6- to 10-membered aryl or 5- to 10-membered heteroaryl, that is,

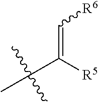

constitutes

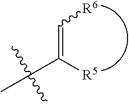

as a 6- to 10-membered aryl or 5- to 10-membered heteroaryl. The same applies to other similar situations.

‘R⁵, R¹⁴ together with the fragment to which R⁵, R¹⁴ are attached form a 4- to 9-membered aliphatic heterocyclyl’ means that R⁵, R¹⁴ and the B² (NR¹⁴) and carbonyl groups between them together form a 4- to 9-membered aliphatic heterocyclyl, that is,

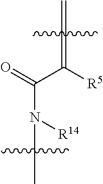

constitutes

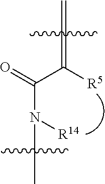

as a 4- to 9-membered aliphatic heterocyclyl. The same applies to other similar situations.

‘R⁶, R¹³ together with the fragment to which R⁶. R¹³ are attached form a 4- to 9-membered cycloalkenyl’ means that R⁶, R¹³ and the B² (CR¹³ or CR¹³R^{13′}), carbonyl, alkenyl fragment together form a 4- to 9-membered cycloalkenyl, that is,

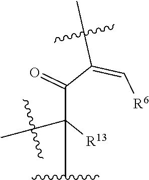

constitutes

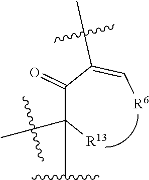

as a 4- to 9-membered cycloalkenyl. The same applies to other similar situations.

The ‘ [symbol] ’ in ring E represents ‘ [symbol] ’ can be a bond or absent. When the ‘ [symbol] ’ in ring E is a bond, R¹¹ is absent means ring E is

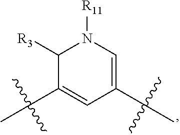

when the ‘ [symbol] ’ is absent, means ring E is

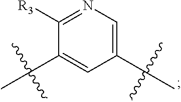

further, when the ‘ [symbol] ’ in ring E is absent, the carbonyl group which formed by R³ and the attached carbon atoms is that, ring E is

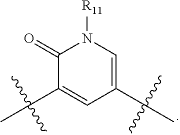

In some embodiments of the present disclosure, the compound of the present disclosure has the structure shown in formula I″ or a tautomer, meso isomer, racemate, enantiomer, diastereomer or mixture thereof, or a pharmaceutically acceptable hydrate, solvate or salt thereof:

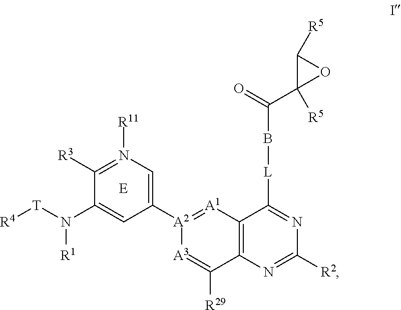

wherein, T is selected from S(O)₂ or C═O.

In some embodiments of the present disclosure, the

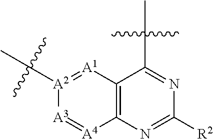

in the compound is selected from the following groups:

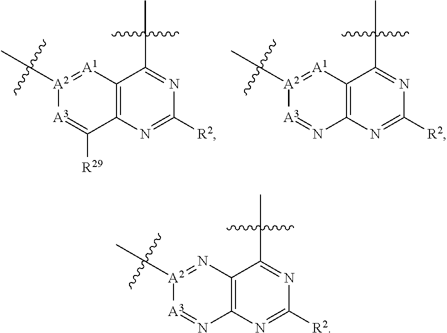

In some embodiments of the present disclosure,

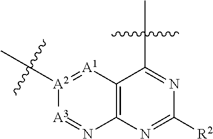

is selected from the following groups:

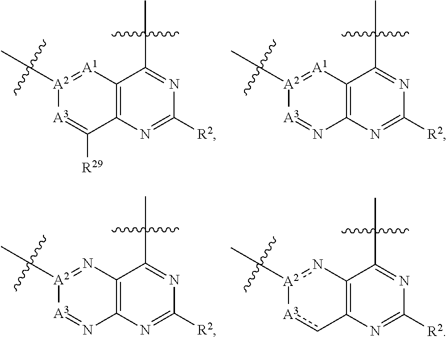

In some embodiments of the present disclosure, the

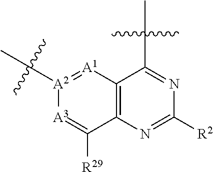

in the compound is selected from the following groups:

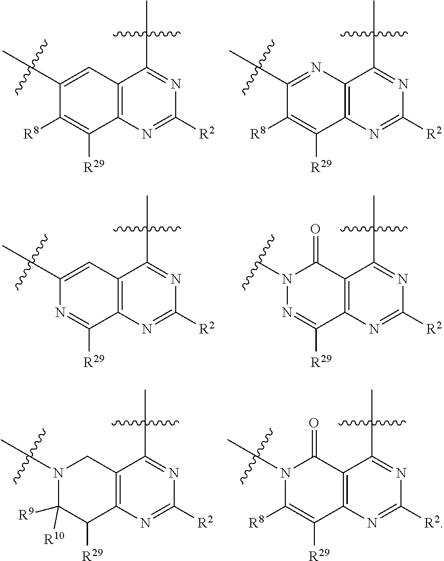

preferably

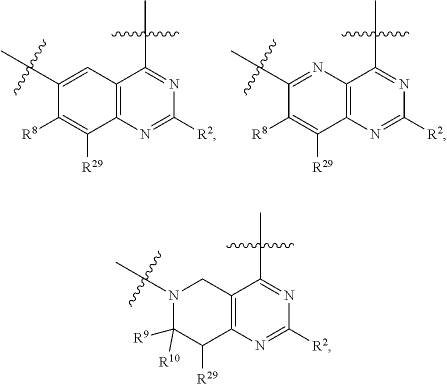

more preferably

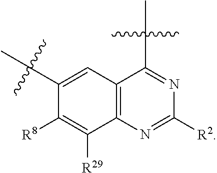

In some embodiments of the present disclosure, the compound of the present disclosure has the structure shown in formula (I′″-1) or formula (I′″-2), or a tautomer, meso isomer, racemate, enantiomer, diastereomer or mixture thereof, or a pharmaceutically acceptable hydrate, solvate or salt thereof:

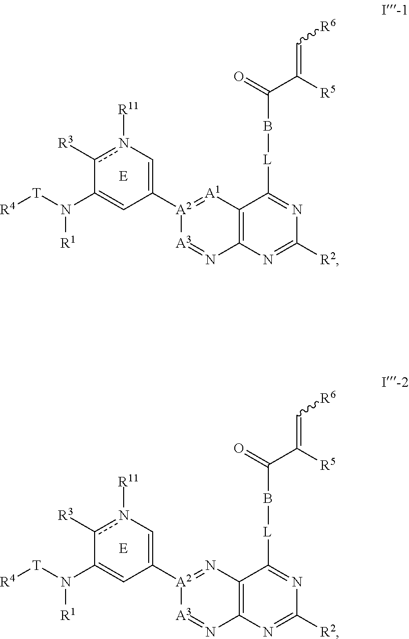

wherein, T is selected from S(O)₂ or C═O.

In some embodiments of the present disclosure, L is a bond, B¹ is N; or L is NR¹⁵, B¹ is CR¹²;
further, when B is

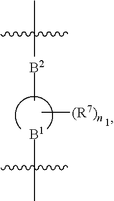

B¹ is N, B² is NR¹⁴.

In some embodiments of the present disclosure, B is

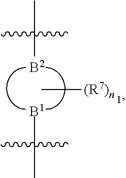

the ring where B¹, B² are located is a 6- to 9-membered nitrogen-containing heterocycloalkyl or a 6- to 9-membered nitrogen-containing heterocycloalkenyl, the cycloalkyl or cycloalkenyl is a monocyclic ring or spirocyclic ring; or, B is

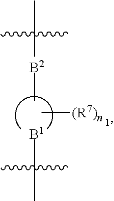

the ring where B¹ is located is a 4- to 6-membered nitrogen-containing monoheterocycloalkyl.

In some embodiments of the present disclosure, B is

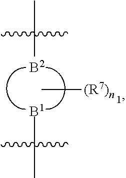

the ring where B¹, B² are located is a 6- to 9-membered nitrogen-containing heterocycloalkyl and is a monocyclic ring or spirocyclic ring: or, B is

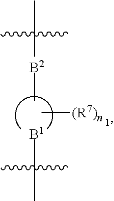

the ring where B¹ is located is a 4- to 6-membered nitrogen-containing monoheterocycloalkyl;

In some embodiments of the present disclosure, B is

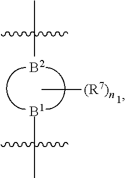

the ring where B¹, B² are located is a 5- to 6-membered nitrogen-containing monoheterocycloalkyl, 6-membered nitrogen-containing monoheterocycloalkenyl or 8- to 9-membered nitrogen-containing spiroheterocycloalkyl; or B is

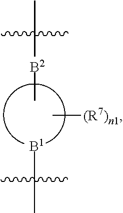

the ring where B¹ is located is a 4-membered nitrogen-containing monoheterocycloalkyl; further, B is

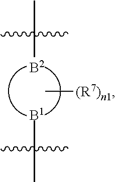

the ring where B¹, B² are located is a 6-membered nitrogen-containing monoheterocycloalkyl or 8- to 9-membered nitrogen-containing spiroheterocycloalkyl; or B is

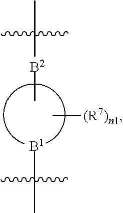

the ring where B¹ is located is a 4-membered nitrogen-containing monoheterocycloalkyl; further,

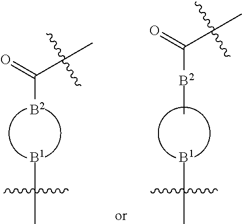

is selected from the following groups:

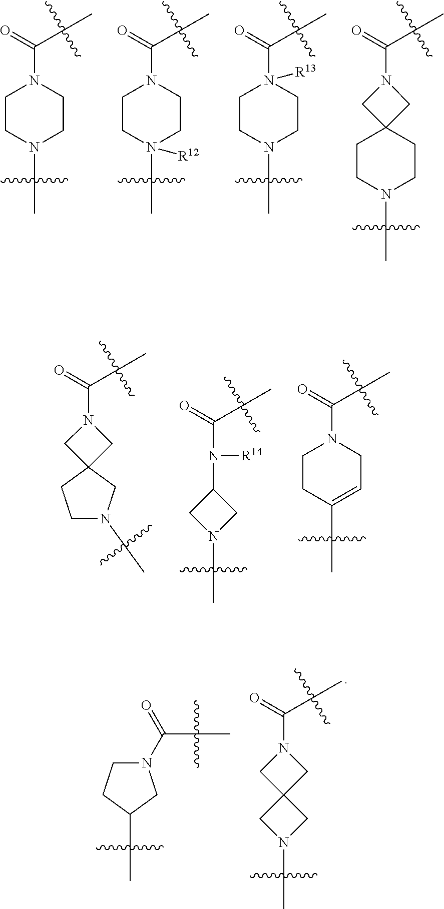

In some embodiments of the present disclosure,

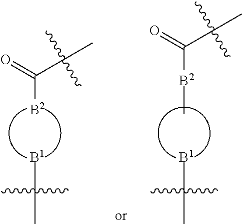

is selected from the following groups:

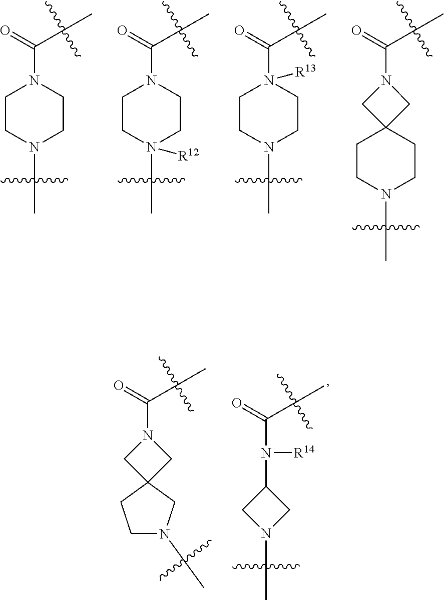

R¹² is selected from hydrogen, halogen, C1˜C6 alkyl, preferably hydrogen, halogen or C1˜C3 alkyl, more preferably hydrogen;
R¹³ is selected from hydrogen, halogen or C1˜C6 alkyl, preferably hydrogen, halogen or C1˜C3 alkyl, or R³ and R⁶ together with the fragment to which R¹³ and R⁶ are attached form a 4- to 6-membered cycloalkenyl, preferably R¹³ and R⁶ together with the fragment to which R¹³ and R⁶ are attached form a 5-membered cycloalkenyl;
R¹⁴ is selected from hydrogen. C1˜C6 alkyl, C3˜C6 cycloalkyl, preferably hydrogen or C1˜C3 alkyl, wherein the alkyl, cycloalkyl are optionally substituted by one or more substituents selected from the group consisting of: halogen, cyano, hydroxy, amino, C1˜C6 alkyl, C3˜C6 cycloalkyl and 3- to 6-membered aliphatic heterocyclyl, or
R¹⁴ and R⁵ together with the fragment to which R¹⁴ and R⁵ are attached form a 4- to 6-membered aliphatic heterocyclyl, preferably R¹⁴ and R⁵ together with the fragment to which R¹⁴ and R⁵ are attached form a 5-membered aliphatic heterocyclyl;
further,

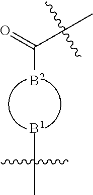

is selected from

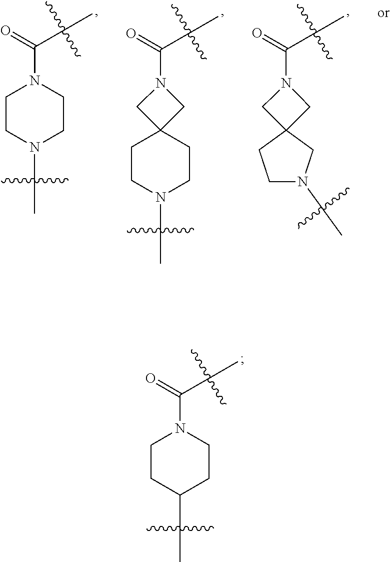 [SMILES: *C(=O)N1CCN(*)CC1 ; *C(=O)N1CC2(CCN(*)CC2)C1 ; *C(=O)N1CC2(CCN(*)C2)C1 ; *C(=O)N1CCC(*)CC1]

preferably

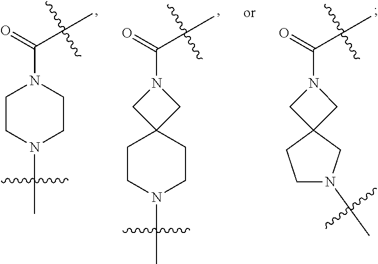 [SMILES: *C(=O)N1CCN(*)CC1 ; *C(=O)N1CC2(CCN(*)CC2)C1 ; *C(=O)N1CC2(CCN(*)C2)C1]

more preferably,

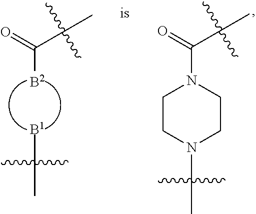

In some embodiments of the present disclosure, B is

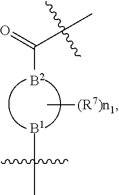

the ring where B¹, B² are located is a 6- to 9-membered nitrogen-containing bridged heterocycloalkyl, preferably 7- to 8-membered nitrogen-containing bridged heterocycloalkyl, more preferably 8-membered nitrogen-containing bridged heterocycloalkyl;
or B is

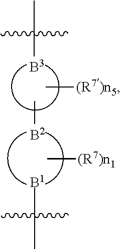

the ring where B¹, B² are located is a 5- to 8-membered nitrogen-containing heterocycloalkyl an is a monocyclic ring, spirocyclic ring or bridged cyclic ring, preferably 5- to 8-membered nitrogen-containing monoheterocycloalkyl, more preferably 6- to 7-membered nitrogen-containing monoheterocycloalkyl; the ring where B³ is located is a 4- to 8-membered nitrogen-containing heterocycloalkyl and is a monocyclic ring, spirocyclic ring or bridged cyclic ring, preferably 4- to 8-membered nitrogen-containing monoheterocycloalkyl, more preferably 4- to 6-membered nitrogen-containing monoheterocycloalkyl; further, R¹², R^{12′}, R^{13″} are independently selected from hydrogen, halogen or C1˜C6 alkyl, preferably hydrogen, halogen or C1˜C3 alkyl, more preferably hydrogen;
further, each of B¹, B², B³ is N;
further,

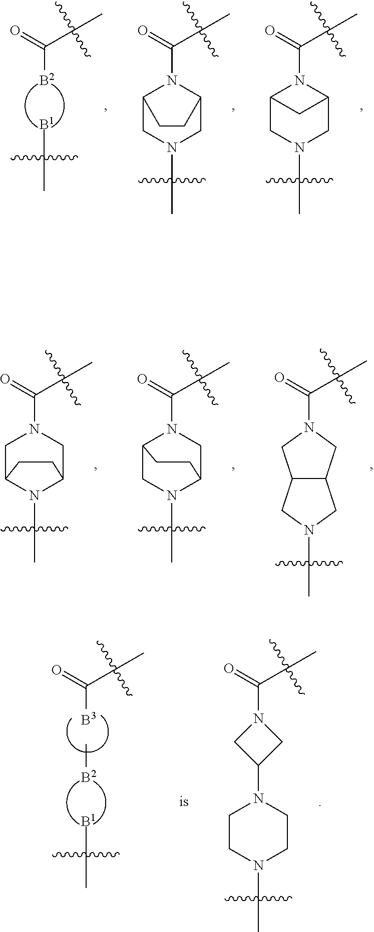

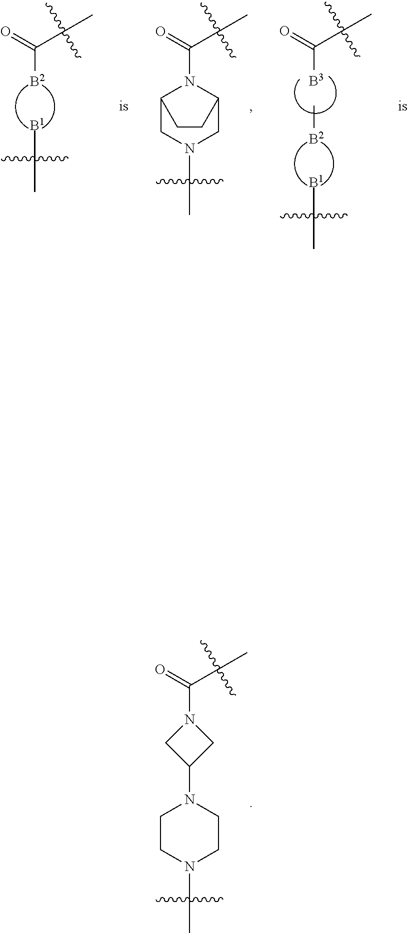

In some embodiments of the present disclosure, the compound of the present disclosure has the structure shown in formula II or formula III, or a tautomer, meso isomer, racemate, enantiomer, diastereomer or mixture thereof, or a pharmaceutically acceptable hydrate, solvate or salt thereof:

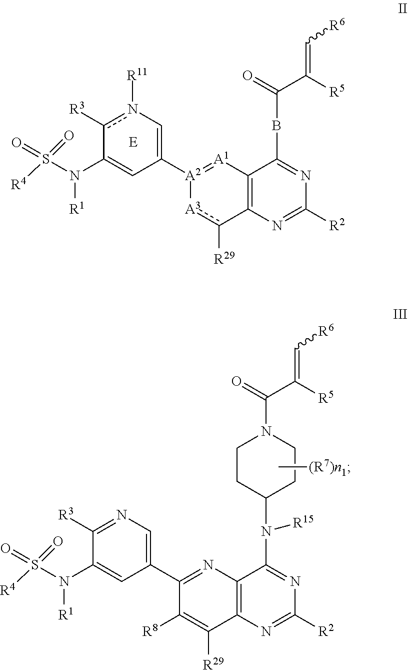

in formula II,

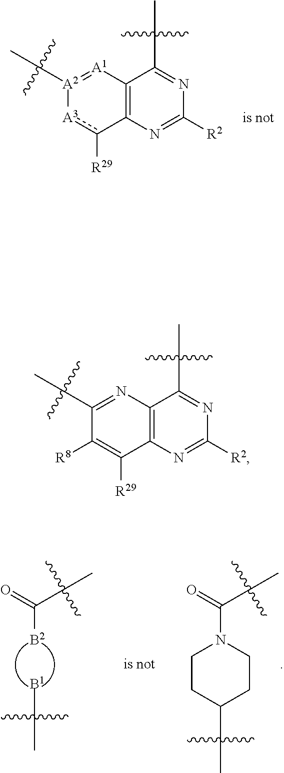

In some embodiments of the present disclosure, the compound of the present disclosure has the structure shown in formula IT or formula III, or a tautomer, meso isomer, racemate, enantiomer, diastereomer or mixture thereof, or a pharmaceutically acceptable hydrate, solvate or salt thereof:

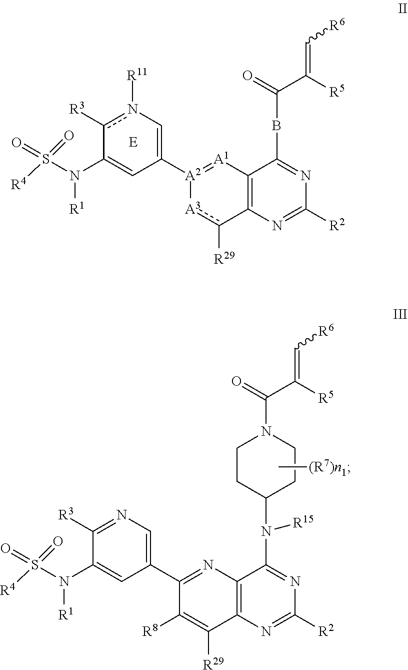

in formula II, when

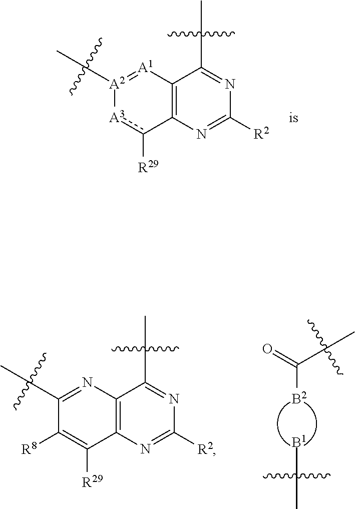

preferably

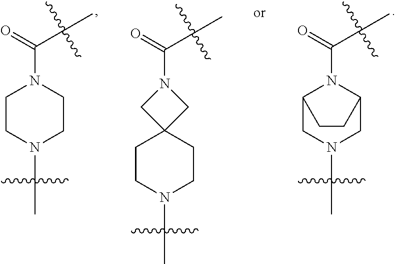 [SMILES: *C(=O)N1CCN(*)CC1 ; *C(=O)N1CC2(CCN(*)CC2)C1 ; *C(=O)N1C2CCC1CN(*)C2]

In some embodiments of the present disclosure, the compound of the present disclosure has the structure shown in formula IV or formula V, or a tautomer, meso isomer, racemate, enantiomer, diastereomer or mixture thereof, or a pharmaceutically acceptable hydrate, solvate or salt thereof:

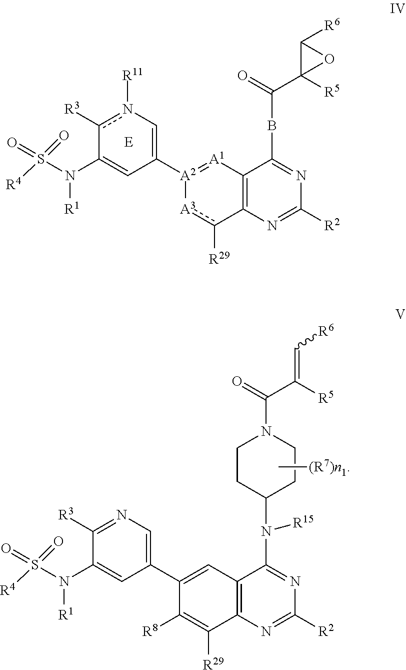

In some embodiments of the present disclosure, n₁ is selected from 0, 1, 2 or 3, preferably 0, 1 or 2, more preferably 0 or 1.

In some embodiments of the present disclosure, R¹, R¹¹, R³ are independently selected from hydrogen, halogen, C1˜C6 alkyl or C3˜C6 cycloalkyl, wherein the alkyl, cycloalkyl are optionally substituted by one or more substituents selected from the group consisting of: halogen, cyano, hydroxy, amino, C1˜C6 alkyl, C3˜C6 cycloalkyl and 3- to 6-membered aliphatic heterocyclyl; when the ‘ [symbol] ’ in ring E is a bond, R¹¹ is absent; further, R¹, R¹¹, R¹⁵ are independently selected from hydrogen, halogen or C1-˜C3 alkyl, wherein the alkyl, cycloalkyl are optionally substituted by one or more substituents selected from the group consisting of: halogen, cyano, hydroxy, amino, C1˜C3 alkyl and C3˜C6 cycloalkyl;
further, R¹, R¹⁵ are independently selected from hydrogen or methyl, preferably hydrogen; R¹¹ is selected from hydrogen or methyl, preferably methyl, when the ‘ [symbol] ’ in ring E is a bond, R¹¹ is absent.

In some embodiments of the present disclosure, the compound of the present disclosure has the structure shown in formula II′ or formula III′ or a tautomer, meso isomer, racemate, enantiomer, diastereomer or mixture thereof, or a pharmaceutically acceptable hydrate, solvate or salt thereof:

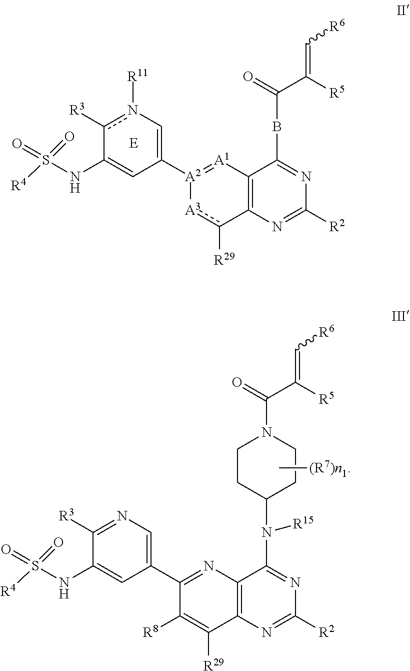

In some embodiments of the present disclosure, the compound of the present disclosure has the structure shown in formula IV′ or formula V′ or a tautomer, meso isomer, racemate, enantiomer, diastereomer or mixture thereof, or a pharmaceutically acceptable hydrate, solvate or salt thereof:

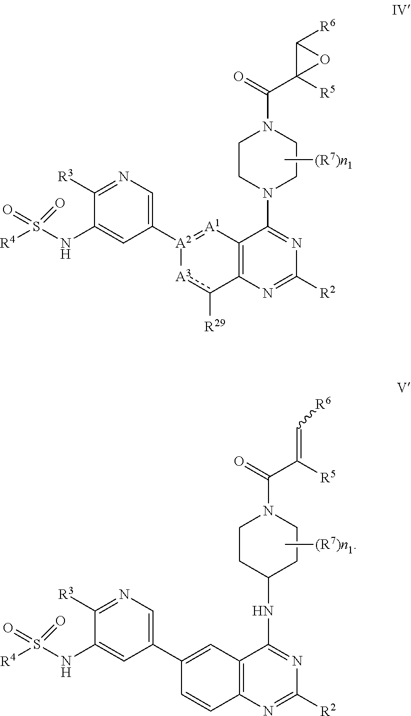

In some embodiments of the present disclosure, the compound of the present disclosure has the structure shown in formula VI, VII, VIII or LX or a tautomer, meso isomer, racemate, enantiomer, diastereomer or mixture thereof, or a pharmaceutically acceptable hydrate, solvate or salt thereof:

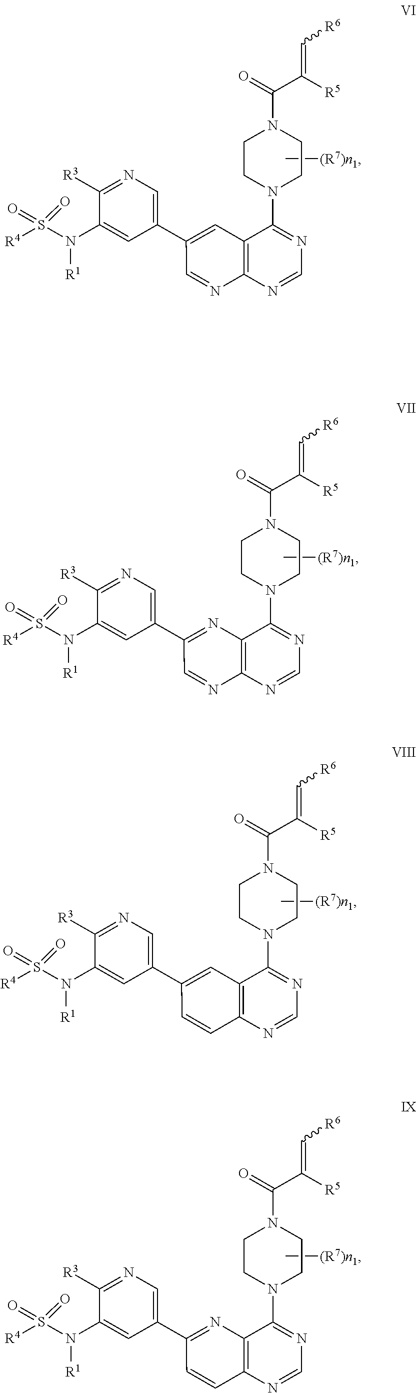

wherein n₁ is selected from 0, 1, 2 or 3, preferably 0, 1 or 2, more preferably 0 or 1.

In some embodiments of the present disclosure, R⁵, R⁶ are independently selected from hydrogen, halogen, C1˜C6 alkyl, C3˜C6 cycloalkyl, 3- to 6-membered aliphatic heterocyclyl, 6- to 10-membered aryl, 5- to 10-membered heteroaryl, —C(O)R¹⁶, —S(O)R¹⁶, —S(O)₂R¹⁶, —P(O)R¹⁶R¹⁷, —C(O)NR^{b}R^{c} or —C(O)OR^{a}, wherein the alkyl, cycloalkyl, aliphatic heterocyclyl, aryl, heteroaryl are optionally substituted by one or more R¹⁸;
each occurrence of R¹⁸ is independently selected from halogen, cyano, C1˜C6 alkyl, C3˜C6 cycloalkyl, —OR^{a} or —NR^{b}R^{c};
or R⁵, R⁶ together with the atoms to which R⁵, R⁶ are attached form

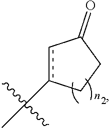

n₂ is selected from 0, 1, 2, 3 or 4;
or R⁵, R¹⁴ together with the fragment to which R⁵, R¹⁴ are attached form

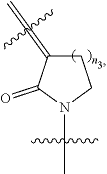

n₃ is selected from 0, 1, 2, 3 or 4;
or R⁶, R¹³ together with the fragment to which R⁶, R¹³ are attached form

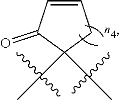

n₄ is selected from 0, 1, 2, 3 or 4;
further, R⁵, R⁶ are independently selected from hydrogen, halogen, C1˜C3 alkyl, 5- to 6-membered heteroaryl, —C(O)R¹⁶, —S(O)₂R¹⁶ or —P(O)R¹⁶R¹⁷, wherein the alkyl, heteroaryl are optionally substituted by one or more R¹⁸;
each occurrence of R¹⁸ is independently selected from halogen, C1˜C3 alkyl, —OR^{a} or —NR^{b}R^{c};
or R⁶, R¹³ together with the atoms to which R⁶, R¹³ are attached form

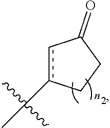

n₂ is selected from 1, 2 or 3;
or R⁵, R⁶ together with the fragment to which R⁵, R⁶ are attached form

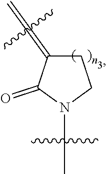

n₃ is selected from 1, 2 or 3;
or R⁶, R¹³ together with the fragment to which R⁶, R¹³ are attached form

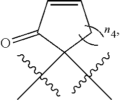

n₄ is selected from 1, 2 or 3;
further, R⁵, R⁶ are independently selected from H, F, —C(O)CH₃, —C(O)CH₂CH₂CH₃, —CH₂N(CH₃)₂, —CH₂F, —S(O)₂CH₃, —P(O)(CH₃)₂ or

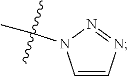 [SMILES: *n1ccnn1]

or R⁵, R⁶ together with the atoms to which R⁵, R⁶ are attached form

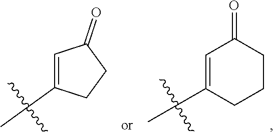 [SMILES: *C1=CC(=O)CC1 ; *C1=CC(=O)CCC1]

or R⁵ or R¹⁴ together with the fragment to which R⁵, R¹⁴ are attached form

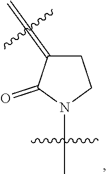 [SMILES: *N1CCC(=*)C1=O]

or R⁶, R¹³ together with the fragment to which R⁶, R¹³ are attached form

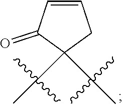 [SMILES: *C1(*)CC=CC1=O]

further, R⁵, R⁶ are independently selected from H, F, —C(O)CH₃, —C(O)CH₂CH₂CH₃, —CH₂F or

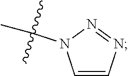 [SMILES: *n1ccnn1]

further, R⁵ is selected from H or F, R⁶ is selected from H, —C(O)CH₃, —C(O)CH₂CH₂CH₃, —CH₂F or

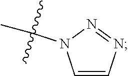 [SMILES: *n1ccnn1]

further, the configuration of

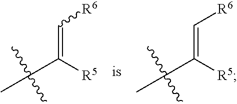

In some embodiments of the present disclosure, R⁵, R⁶ are independently selected from hydrogen, halogen, C1˜C6 alkyl, C3˜C6 cycloalkyl, 3- to 6-membered aliphatic heterocyclyl, 6- to 10-membered aryl, 5- to 10-membered heteroaryl, —C(O)R¹⁶, —S(O)R¹⁶, —S(O)₂R¹⁶, —P(O)R¹⁶R¹⁷, —C(O)NR^{b}R^{c}, —C(O)OR^{a}, —OR^{a} or —NR^{b}R^{c}, wherein the alkyl, cycloalkyl, aliphatic heterocyclyl, aryl, heteroaryl are optionally substituted by one or more R¹⁸;
each occurrence of R¹⁸ is independently selected from halogen, cyano, C1˜C6 alkyl, C3˜C6 cycloalkyl, —OR^{a} or —NR^{b}R^{c};
or Z¹ is a bond, R⁵, R⁶ together with the atoms to which R⁵, R⁶ are attached form a 6- to 10-membered aryl, wherein the aryl is optionally substituted by one or more substituents selected from the group consisting of: halogen, cyano, hydroxy, amino, C1˜C6 alkyl, C3˜C6 cycloalkyl and 3- to 6-membered aliphatic heterocyclyl;
further, R⁵, R⁶ are independently selected from hydrogen, halogen, C1˜C3 alkyl, 5- to 6-membered heteroaryl, —C(O)R¹⁶, —S(O)₂R¹⁶, —P(O)R¹⁶R¹⁷, —C(O)NR^{b}R^{c}, —C(O)OR^{a} or —OR^{a}, wherein the alkyl, heteroaryl are optionally substituted by one or more R¹⁸;
further, R⁵, R⁶ are independently selected from hydrogen, halogen, C1˜C3 alkyl, 5- to 6-membered heteroaryl, —C(O)R¹⁶, —S(O)₂R¹⁶, —P(O)R¹⁶R¹⁷, —C(O)OR^{a} or —OR^{a}, wherein the alkyl, heteroaryl are optionally substituted by one or more R¹⁸;
each occurrence of R¹⁸ is independently selected from halogen, C1˜C3 alkyl, —OR^{a} or —NR^{b}R^{c};
or Z¹ is a bond, R⁵, R⁶ together with the atoms to which R⁵, R⁶ are attached form a phenyl, wherein the phenyl is optionally substituted by 1˜2 following substituents: halogen, cyano, hydroxy, amino, C1˜C3 alkyl;
further, R⁵, R⁶ are independently selected from hydrogen, halogen, C1˜C3 alkyl, —C(O)R¹⁶, —S(O)₂R¹⁶, —P(O)R¹⁶R¹⁷, —C(O)O—C1˜C3 alkyl, —O—C1˜C3 alkyl, 1H-1,2,3-triazolyl or oxazolyl, wherein the alkyl, 1H-1,2,3-triazolyl, oxazolyl are optionally substituted by 1˜3 R¹⁸;
each occurrence of R¹⁸ is independently selected from halogen, C1˜C3 alkyl, —OR^{a} or —NR^{b}R^{c};
or Z¹ is a bond, R⁵, R⁶ together with the atoms to which R⁵, R⁶ are attached from a phenyl substituted by one hydroxy;
further, R⁵, R⁶ are independently selected from H, F, CN, —CH₂OCH₃, —C(CH₃)₃, —CH(OH)CH₃, C(O)CF₃, —C(O)CH₂CH₃, —C(O)CH₃, —C(O)NHCH₃, —C(O)CH₂CH₂CH₃, —CH₂N(CH₃)₂, —CH₂F, —S(O)₂CH₃, —P(O)(CH₃)₂,

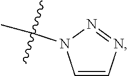 [SMILES: *n1ccnn1]

—CHCH₃CH₃, —CH₂OCH₃, CF₃,

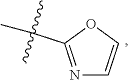 [SMILES: *c1ncco1]

—C(O)OCH₃ or —OCH₃;
further, R⁵, R⁶ are independently selected from H, F, —C(O)CH₃, —C(O)CH₂CH₂CH₃; —CH₂N(CH₃)₂, —CH₂F, —S(O)₂CH₃, —P(O)(CH)₂,

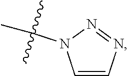 [SMILES: *n1ccnn1]

—CHCH₃CH₃, —CH₂OCH₃, CF₃,

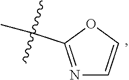 [SMILES: *c1ncco1]

—C(O)OCH₃ or —OCH₃;
or Z¹ is a bond, R⁵, R⁶ together with the atoms to which R⁵, R⁶ are attached form

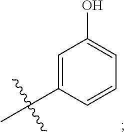 [SMILES: *c1cccc(O)c1]

further, R⁵ is selected from H, F, CN, —CH(CH₃)₂, CH(OH)CH₃, —CH₂OCH₃, —OCH₃ or —C(O)CH₃, R⁶ is selected
from H, —C(CH₃)₃, —C(O)CH₃, —C(O)CH₂CH₂CH₃, —C(O)NHCH₃, —CH₂N(CH₃)₂, —CH₂F,

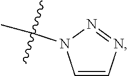 [SMILES: *n1ccnn1]

—CH₂OCH₃, CF₃,

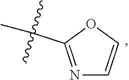 [SMILES: *c1ncco1]

—C(O)OCH₃, —P(O)(CH₃), —S(O)₂CH₃, —C(O)CF₃ or —C(O)CH₂CH₃.
further, R⁵ is selected from H, F, —CHCH₃CH₃, —CH₂OCH₃ or —OCH₃, R⁶ is selected from H, —C(O)CH₃, —C(O)CH₂CH₂CH₃, —CH₂F,

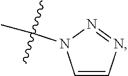 [SMILES: *n1ccnn1]

—CH₂OCH₃, CF₃,

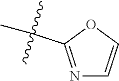 [SMILES: *c1ncco1]

or —C(O)OCH₃.

In some embodiments, Z¹ is a bond; R⁵ is H; R⁶ is selected from H, —C(CH₃)₃, —C(O)CH₃, —C(O)CH₂CH₂CH₃, —C(O)NHCH₃, —CH₂N(CH₃)₂, —CH₂F,

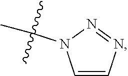 [SMILES: *n1ccnn1]

—CH₂OCH₃, CF₃,

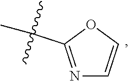 [SMILES: *c1ncco1]

—C(O)OCH₃, —P(O)(CH₃), —S(O)₂CH₃ or —C(O)CF₃; preferably, R⁶ is selected from H and —C(O)CH₃;
further preferably,

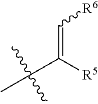

is

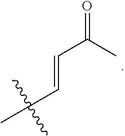 [SMILES: */C=C/C(C)=O]

In some embodiments of the present disclosure, each occurrence of R^{a}, R^{b}, R^{c} is independently selected from H, C1˜C6 alkyl or —C(O)R¹³, wherein the alkyl is optionally substituted by one or more substituents selected from the group consisting of: halogen, cyano, hydroxy, amino and C1˜C6 alkyl;
further, each occurrence of R^{a}, R^{b}, R^{c} is independently selected from H or C1˜C3 alkyl, wherein the alkyl is optionally substituted by one or more of the following substituents: halogen, C1˜C3 alkyl;
further, each occurrence of R^{a}, R^{b}, R^{c} is independently selected from H or C1˜C3 alkyl, preferably H or methyl;
more preferably methyl.

In some embodiments of the present disclosure, R² is selected from hydrogen, halogen, cyano, C1˜C6 alkyl, C3˜C6 cycloalkyl, —OR^{d} or —NR^{e}R^{f}, wherein the alkyl, cycloalkyl are optionally substituted by one or more R¹⁴,
each occurrence of R¹⁴ is independently selected from halogen, cyano, C1˜C6 alkyl, C3˜C6 cycloalkyl, —OR^{d} or —NR^{e}R^{f};
further, R² is selected from hydrogen, halogen, cyano, C1˜C3 alkyl, —OR^{d} or —NR^{e}R^{f}, wherein the alkyl is optionally substituted by one or more R¹⁴, each occurrence of R¹⁴ is independently selected from halogen, cyano, C1˜C3 alkyl, —OR^{d} or —NR^{e}R^{f};
further, R² is hydrogen.

In some embodiments of the present disclosure, each occurrence of R^{d}, R^{e}, R^{f} is independently selected from H, C1˜C6 alkyl or C3˜C6 cycloalkyl, wherein the alkyl, cycloalkyl are optionally substituted by one or more substituents selected from the group consisting of: halogen, cyano, hydroxy, amino, C1˜C6 alkyl, C3˜C6 cycloalkyl and alkyl substituted or unsubstituted C3˜C6 heterocycloalkyl;
further, each occurrence of R^{d}, R^{e}, R^{f} is independently selected from H or C1˜C3 alkyl, wherein the alkyl is optionally substituted by one or more substituents selected from the group consisting of: halogen, C1˜C3 alkyl and alkyl substituted or unsubstituted 5-membered nitrogen-containing heterocycloalkyl.

In some embodiments of the present disclosure, each occurrence of R^{d}, R^{e}, R^{f} is independently selected from H, C1˜C6 alkyl or C3˜C6 cycloalkyl, wherein the alkyl, cycloalkyl are optionally substituted by one or more of the following substituents: halogen, cyano, hydroxy, amino, C1˜C6 alkyl, C3˜C6 cycloalkyl, C3˜C6 heterocycloalkyl;
further, each occurrence of R^{d}, R^{e}, R^{f} is independently selected from H or C1˜C3 alkyl, wherein the alkyl is optionally substituted by one or more of the following substituents: halogen, C1˜C3 alkyl, 5-membered nitrogen-containing heterocycloalkyl;
further, each occurrence of R^{d}, R^{e}, R^{f} is independently selected from H or C1˜C3 alkyl, the alkyl is optionally substituted by one or more of the following substituents: halogen, C1˜C3 alkyl,

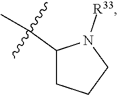

R³³ is selected from H or C1˜C6 alkyl, preferably C1˜C3 alkyl, more preferably methyl.

In some embodiments of the present disclosure, R³ is selected from hydrogen, halogen, cyano, C1˜C6 alkyl, C3˜C6 cycloalkyl, 3- to 6-membered aliphatic heterocyclyl, —OR^{g}, —C(O)OR^{g}, —SR^{g} or —NR^{h}Rⁱ, when the ‘ [symbol] ’ in ring E is absent, R³ can also be a carbonyl group that together formed by the connected carbon atom, wherein the alkyl, cycloalkyl, aliphatic heterocyclyl are optionally substituted by one or more R²⁵, each occurrence of the R²⁵ is independently selected from halogen, cyano, C1˜C6 alkyl, C3˜C6 cycloalkyl, —OR^{g} or —NR^{h}Rⁱ;
further, R³ is selected from hydrogen, halogen, cyano, C1˜C6 alkyl, C3˜C6 cycloalkyl, 3- to 6-membered aliphatic heterocyclyl, —OR^{g} or —NR^{h}Rⁱ, when the ‘ [symbol] ’ in ring E is absent, R³ can also be a carbonyl group that together formed by the connected carbon atom, wherein the alkyl, cycloalkyl, aliphatic heterocyclyl are optionally substituted by one or more R²⁵, each occurrence of the R²⁵ is independently selected from halogen, cyano, C1˜C6 alkyl, C3˜C6 cycloalkyl, —OR^{g} or —NR^{h}Rⁱ;
further, R³ is selected from hydrogen, halogen, cyano, C1˜C6 alkyl, —OR^{g}, —C(O)OR^{g}, —SR^{g} or —NR^{h}Rⁱ, when the ‘ [symbol] ’ in ring E is absent, R³ can also be a carbonyl group that together formed by the connected carbon atom, wherein the alkyl is optionally substituted by one or more halogen or —OH;
further, R³ is selected from hydrogen, halogen, C1˜C3 alkyl, —OR^{g} or —NR^{h}Rⁱ, when the ‘ [symbol] ’ in ring E is absent, R³ can also be a carbonyl group that together formed by the connected carbon atom, wherein the alkyl is optionally substituted by one or more R²⁵, each occurrence of R²⁵ is independently selected from halogen, cyano, C1˜C3 alkyl, —OR^{g} or —NR^{h}Rⁱ;
further. R³ is selected from hydrogen, F, Cl, Br, methyl, —OR^{g} or —NR^{h}Rⁱ, when the ‘ [symbol] ’ in ring E is absent, R³ forms carbonyl group together with the connected carbon atom;
further, R³ is selected from hydrogen, Cl, methyl or —OR^{g}, when the ‘ [symbol] ’ in ring E is absent, R³ forms carbonyl group together with the connected carbon atom;
further, R³ is selected from hydrogen, Cl or —OR⁸;
further, R³ is selected from hydrogen, Cl, —CF₃, —OCH₃, —OCH₂CH₃, —OCF₃, —OCHF₂, —N(CH₃)₂, —CH₃, —CH₂OH, —OCH₂CF₃, —OH, —NHCH₃, —SCH₃, —OCD₃, —CN or —C(O)OCH₃.

Further, R³ is selected from hydrogen, —OCH₃, —OCH₂CH₃, —N(CH₃)₂, —CH₂OH, —OCH₂CF₃, —SCH₃, —OCD₃, —CN or —C(O)OCH₃; preferably, R³ is —OCH₃.

In some embodiments of the present disclosure, each occurrence of R^{g}, R^{h}, Rⁱ is independently selected from H, C1˜C6 alkyl or C3˜C6 cycloalkyl, wherein the alkyl, cycloalkyl are optionally substituted by one or more substituents selected from the group consisting of: halogen, cyano, hydroxy, amino and C1˜C6 alkyl; further, each occurrence of R^{g}, R^{h}, Rⁱ is independently selected from H, C1˜C3 alkyl, cyclopropyl or cyclopentyl, wherein the alkyl, cyclopropyl are optionally substituted by one or more of the following substituents: halogen, C1˜3 alkyl;
further, each occurrence of R^{g}, R^{h}, Rⁱ is independently selected from H, methyl, cyclopropyl or cyclopentyl, preferably methyl.

In some embodiments of the present disclosure, each occurrence of R^{g}, R^{h}, Rⁱ is independently selected from H, methyl, cyclopropyl, cyclopentyl, ethyl, trifluoromethyl, trifluoroethyl, difluoromethyl or deuterated methyl.

In some embodiments of the present disclosure, R^{g}, R^{h}, Rⁱ is independently selected from H, methyl, cyclopropyl, cyclopentyl or ethyl;
preferably, each occurrence of R^{g}, R^{h}, Rⁱ is independently selected from H, methyl, cyclopropyl or ethyl.

In some embodiments of the present disclosure, R⁸, R⁹, R¹⁰, R²⁹ are independently selected from hydrogen, halogen, cyano, C1˜C6 alkyl, C3˜C6 cycloalkyl, —OR^{q} or —NR^{r}R^{s}, wherein the alkyl, cycloalkyl are optionally substituted by one or more R³¹;
each occurrence of R³¹ is independently selected from halogen, cyano, C1˜C6 alkyl, C3˜C6 cycloalkyl, —OR^{q}, —NR^{r}R^{s}, —C(O)R¹⁵, —C(O)NR^{r}R^{s} or —C(O)OR^{q}; further, R⁸, R⁹, R¹⁰, R²⁹ are independently selected from hydrogen, halogen, cyano, C1˜C3 alkyl or —OR^{q}, wherein the alkyl, cycloalkyl are optionally substituted by one or more R³¹;
each occurrence of R³¹ is independently selected from halogen, C1˜C3 alkyl or —OR^{q};
further, R⁸, R²⁹ are independently selected from hydrogen, F, Cl, Br, C1˜C3 alkyl, halogenated C1˜C3 alkyl, —CN or —OR^{q}, preferably hydrogen, Cl or OH;
R⁹, R¹⁰ is hydrogen;
further, R⁸, R⁹, R¹⁰, R²⁹ are independently selected from hydrogen, halogen, C1˜C3 alkyl or —OR^{q}, wherein the alkyl, cycloalkyl are optionally substituted by one or more R³¹;
each occurrence of R³¹ is independently selected from halogen, C1˜C3 alkyl or —OR^{q}; further, R⁸, R²⁹ are independently selected from hydrogen, F, Cl, Br, C1˜C3 alkyl or —OR^{q}, preferably hydrogen, Cl, OH;
R⁹, R¹⁰ is hydrogen; further, R⁸ is selected from hydrogen, Cl or —CH₃; preferably, R⁸ is selected from hydrogen or Cl, preferably hydrogen; R²⁹ is selected from hydrogen, OH, —CN, F or —CF₃, preferably, R²⁹ is selected from hydrogen, OH, —CN or F; preferably, R²⁹ is selected form hydrogen or OH, preferably hydrogen.

In some embodiments of the present disclosure, each occurrence of R^{q}, R^{r}, R^{s} is independently selected from H, C1˜C6 alkyl or C3˜C6 cycloalkyl, wherein the alkyl, cycloalkyl are optionally substituted by one or more of the following substituents: halogen, cyano, hydroxy, amino, C1˜C6 alkyl;
further, each occurrence of R^{q}, R^{r}, R^{s} is independently selected from H or C1˜C3 alkyl, preferably H.

In some embodiments of the present disclosure, R⁴ is selected from C1˜C6 alkyl, C3-C9 cycloalkyl, 3- to 6-membered heterocycloalkyl, 6- to 10-membered aryl or 5- to 10-membered heteroaryl, wherein the alkyl, cycloalkyl, heterocycloalkyl, aryl, heteroaryl are optionally substituted by one or more R²⁶;
each occurrence of R²⁶ is independently selected from halogen, cyano, C1˜C6 alkyl, C3˜C6 cycloalkyl, —OR^{j}, —NR^{k}R^{m}, —C(O)R²⁷, —C(O)NR^{k}R^{m} or —C(O)OR^{j}, wherein the alkyl, cycloalkyl are optionally substituted by one or more R²⁶;
each occurrence of R²⁸ is independently selected from halogen, cyano, alkyl, cycloalkyl, —OR^{j} or —NR^{k}R^{m};
further, R⁴ is selected from C1˜C3 alkyl, C3˜C6 cycloalkyl, phenyl, 5- to 6-membered nitrogen-containing heteroaryl or 5- to 6-membered sulphur-containing heteroaryl, wherein the alkyl, cycloalkyl, aryl, nitrogen-containing heteroaryl, sulphur-containing heteroaryl are optionally substituted by 1˜3 R²⁶;
each occurrence of R²⁶ is independently selected from halogen, cyano, C1˜C3 alkyl, C3˜C6 cycloalkyl, —OR^{j} or —NR^{k}R^{m};

In some embodiments, R⁴ is selected from C1˜C3 alkyl, C3˜C6 cycloalkyl, phenyl, naphthyl, 5- to 6-membered nitrogen-containing heteroaryl or 5- to 6-membered sulphur-containing heteroaryl, wherein the alkyl, cycloalkyl, aryl, nitrogen-containing heteroaryl, sulphur-containing heteroaryl are optionally substituted by 1˜3 R²⁶;
each occurrence of R²⁶ is independently selected from halogen, cyano, C1˜C3 alkyl, halogenated C1˜C3 alkyl, —O(C1˜C3 alkyl) or —C(O)OH.

In some embodiments, R⁴ is selected from C1˜C3 alkyl, cyclohexyl, phenyl, pyridyl, thienyl, naphthyl, pyrrolyl or thiazolyl, wherein the alkyl, cyclohexyl, phenyl, pyridyl, thienyl, naphthyl, pyrrolyl, thiazolyl are optionally substituted by 1˜2 R²⁶;
each occurrence of R²⁶ is independently selected from F, Cl, Br, cyano, unsubstituted C1˜C3 alkyl or C1˜C3 alkyl substituted by 1 to 3 halogen, —OR^{j} or —C(O)OR^{j}, more preferably F, Cl, methyl, methoxy, cyano, trifluoromethyl, or —COOH;
preferably, each occurrence of R²⁶ is independently selected from F, Cl, Br, cyano, unsubstituted C1˜C3 alkyl or C1˜C3 alkyl substituted by 1 to 3 halogen or —OR^{j}, more preferably F, Cl, methyl, methoxy, cyano or trifluoromethyl.

In some embodiments, R⁴ is selected from C1˜C3 alkyl, cyclohexyl, phenyl, pyrrolyl or thiazolyl, wherein the alkyl, cyclohexyl, phenyl, pyrrolyl, thiazolyl are optionally substituted by 1˜2 R²⁶; each occurrence of R²⁶ is independently selected from F, Cl, Br, C1˜C3 alkyl, —OR^{j}, preferably F, Cl, methyl or methoxy.

In some embodiments, R⁴ is selected from C1˜C3 alkyl, cyclohexyl, phenyl, pyrrolyl or thiazolyl, wherein the alkyl, cyclohexyl, phenyl, pyrrolyl, thiazolyl are optionally substituted by 1˜2 R²⁶;
each occurrence of R²⁶ is independently selected from F, Cl, Br, C1˜C3 alkyl or —OR^{j}, preferably F, Cl, methyl or methoxy.

In some embodiments of the present disclosure, R⁴ is selected from methyl, cyclohexyl,

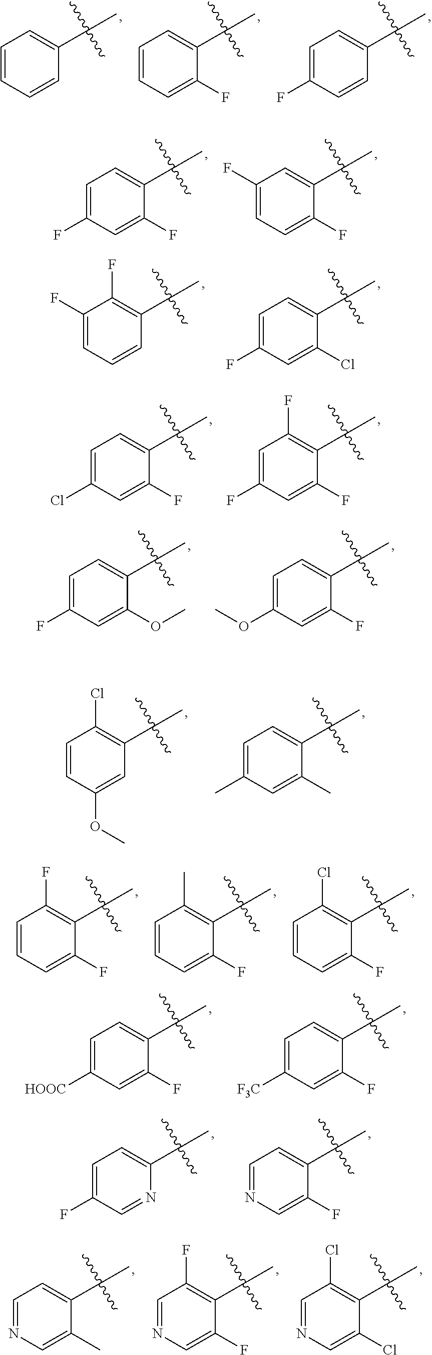

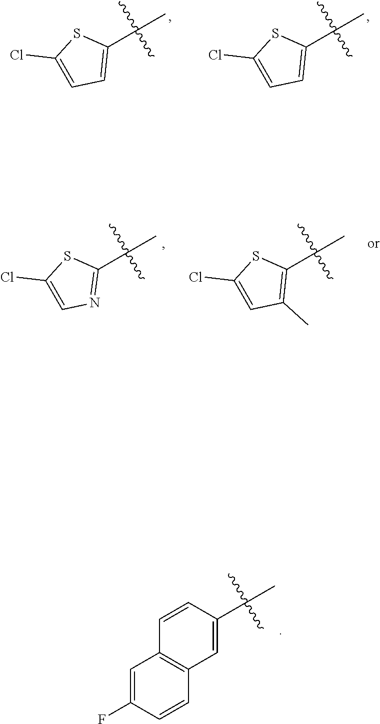

Further, R⁴ is selected from methyl, cyclohexyl,

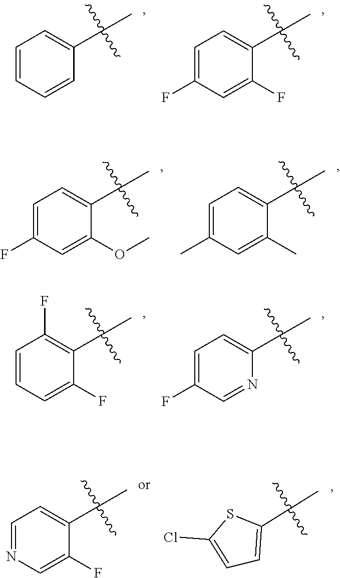

preferably methyl, cyclohexyl,

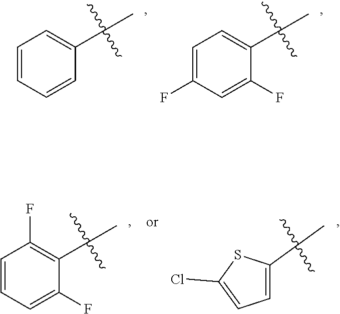 [SMILES: *c1ccccc1 ; *c1ccc(F)cc1F ; *c1c(F)cccc1F ; *c1ccc(Cl)s1]

more preferably

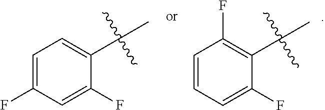 [SMILES: *c1ccc(F)cc1F ; *c1c(F)cccc1F]

In some embodiments of the present disclosure, R⁴ is selected from

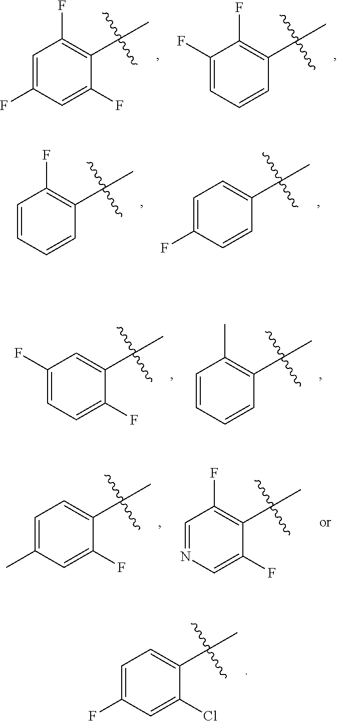

In some embodiments of the present disclosure, each occurrence of R²⁶ is independently selected from halogen, cyano, C1˜C3 alkyl, C3˜C6 cycloalkyl, —OR^{j} or —NR^{k}R^{m}, wherein the alkyl, cycloalkyl are optionally substituted by 1˜3 R²⁸;
each occurrence of R²⁸ is independently selected from halogen, cyano, C1˜C3 alkyl, —OR^{j} or —NR^{k}R^{m};
further, each occurrence of R²⁶ is independently selected from F, Cl, Br, cyano, unsubstituted C1˜C3 alkyl or C1˜C3 alkyl substituted by 1 to 3 halogen, —OR^{j}, more preferably F, Cl, methyl, methoxy, cyano or trifluoromethyl;
further, R⁴ is selected from

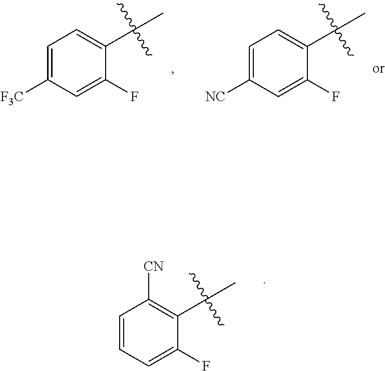 [SMILES: *c1ccc(C(F)(F)F)cc1F ; *c1ccc(C#N)cc1F ; *c1c(F)cccc1C#N]

In some embodiments of the present disclosure, R⁴ is selected from naphthyl or pyrrolyl, wherein the naphthyl, pyrrolyl are optionally substituted by 1˜3 R²⁶; R²⁶ is as defined above;
further, the R⁴ is selected from unsubstituted following group or following group substituted by 1 to 3 R²⁶;

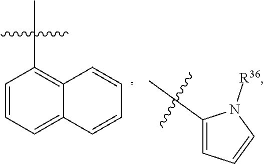

R³⁶ is selected from H or C1˜C6 alkyl, preferably C1˜C3 alkyl, more preferably methyl;
further, the R⁴ is selected from

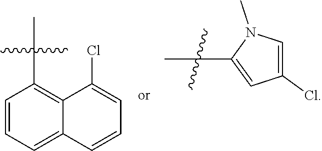

In some embodiments of the present disclosure, each occurrence of R^{j}, R^{k}, R^{m} is independently selected from H, C1˜C6 alkyl or C3˜C6 cycloalkyl, wherein the alkyl, cycloalkyl are optionally substituted by one or more substituents selected from the group consisting of: halogen, cyano, hydroxy, amino and C1˜C6 alkyl; further, each occurrence of R^{j}, R^{k}, R^{m} is independently selected from H or C1˜C3 alkyl, preferably C1˜C3 alkyl, more preferably methyl.

In some embodiments of the present disclosure, each occurrence of R⁷ is independently selected from halogen, cyano, C1˜C6 alkyl, —ORⁿ, —NR^{o}R^{p}, —C(O)R²⁰, —C(O)NR^{o}R^{p} or —C(O)ORⁿ, wherein the alkyl is optionally substituted by one or more R²¹;
each occurrence of R²¹ is independently selected from halogen, cyano, C1˜C6 alkyl, —ORⁿ, —NR^{o}R^{p}, —C(O)R²⁰, —C(O)NR^{o}R^{p} or —C(O)ORⁿ;
further, each occurrence of R⁷ is independently selected from halogen, cyano, C1˜C3 alkyl, —ORⁿ or —NR^{o}R, wherein the alkyl is optionally substituted by one or more R²¹;
each occurrence of R²¹ is independently selected from halogen, cyano, C1˜C3 alkyl, —ORⁿ or —NR^{o}R^{p}; further, each occurrence of R⁷ is independently selected from C1˜C3 alkyl, wherein the alkyl is optionally substituted by one or more R²¹;
each occurrence of R²¹ is independently selected from halogen, cyano or C1˜C3 alkyl, preferably cyano; further, R⁷ is selected from hydrogen, methyl or —CH₂CN;
further,

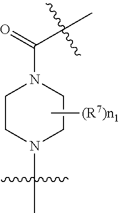

is selected from

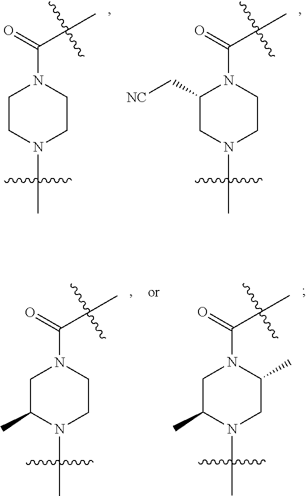

preferably,

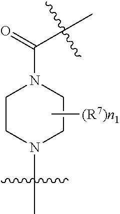

is

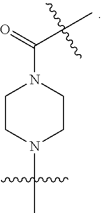 [SMILES: *C(=O)N1CCN(*)CC1]

In some embodiments of the present disclosure, each occurrence of R^{7′} is independently selected from halogen, cyano, C1˜C6 alkyl, —ORⁿ, —NR^{o}R^{p}, —C(O)R²⁰ or —C(O)ORⁿ, wherein the alkyl is optionally substituted by 1˜3 R²¹;
each occurrence of R²¹ is independently selected from halogen, cyano, C1˜C6 alkyl, —ORⁿ or —NR^{o}R^{p};
further, each occurrence of R²¹ is independently selected from halogen, cyano, C1˜C3 alkyl, —ORⁿ or —NR^{o}R^{p}, wherein the alkyl is optionally substituted by 1˜2 R²¹;
each occurrence of R²¹ is independently selected from halogen, cyano, C1˜C3 alkyl, —ORⁿ or —NR^{o}R^{p}; further, n₅ is selected from 0, 1, 2 or 3, preferably 0, 1 or 2, more preferably 0 or 1.

In some embodiments of the present disclosure, each occurrence of Rⁿ, R^{o}, R^{p} is independently selected from H, C1˜C6 alkyl or C3˜C6 cycloalkyl, wherein the alkyl, cycloalkyl are optionally substituted by one or more substituents selected from the group consisting of: halogen, cyano, hydroxy, amino and C1˜C6 alkyl; further, each occurrence of Rⁿ, R^{o}, R^{p} is independently selected from H or C1˜C3 alkyl.

In some embodiments of the present disclosure, each occurrence of R¹⁶, R¹⁷, R¹⁹, R²⁰, R²², R²⁴, R²⁷, R³⁰, R³² is independently selected from H or C1˜C6 alkyl, wherein the alkyl is optionally substituted by one or more substituents selected from the group consisting of: halogen, cyano, hydroxy, amino and C1˜C6 alkyl; further, each occurrence of R¹⁶, R¹⁷, R¹⁹, R²⁰, R²², R²⁴, R²⁷, R³⁰, R³² is independently selected from H or C1˜C3 alkyl, wherein the alkyl is optionally substituted by one or more substituents selected from the group consisting of: halogen, hydroxy, amino and C1˜C3 alkyl;
further, each occurrence of R¹⁶, R¹⁷, R¹⁹, R²⁰, R²², R²⁴, R²⁷, R³⁰, R³² is independently selected from C1˜C3 alkyl, preferably methyl or propyl.

In some embodiments, the compound of the present disclosure has the structure shown in formula II or a tautomer, meso isomer, racemate, enantiomer, diastereomer or mixture thereof, or a pharmaceutically acceptable hydrate, solvate or salt thereof:

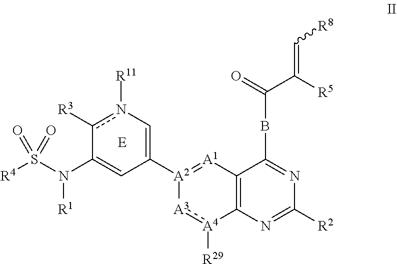

wherein,

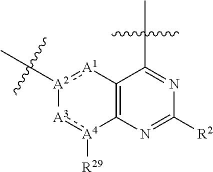

is

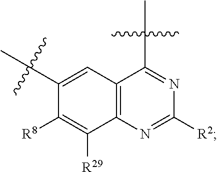

B is

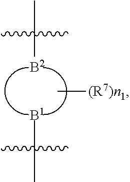

the ring where B¹, B² are located is a 6- to 9-membered nitrogen-containing heterocycloalkyl or a 6- to 9-membered nitrogen-containing heterocycloalkenyl, wherein the cycloalkyl or cycloalkenyl is a monocyclic ring, spirocyclic ring or bridged cyclic ring;
R¹ is H;
R² is H;
the ‘ [symbol] ’ in ring E represents ‘ [symbol] ’ is a bond or absent, when ‘ [symbol] ’ is a bond, R¹¹ is absent, ring E is

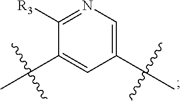

when ‘ [symbol] ’ is absent, ring E is

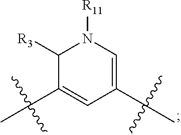

when R¹¹ exists, R¹¹ is selected from hydrogen or methyl;
R⁸ is selected from hydrogen, Cl or —CH₃; preferably R⁸ is hydrogen;
R²⁹ is selected from hydrogen, OH, —CN, F or —CF₃; preferably R²⁹ is hydrogen;
R³ is selected from hydrogen, halogen, cyano, C1˜C6 alkyl, —OR^{g}, —C(O)OR^{g}, —SR^{g} or —NR^{h}Rⁱ, when the ‘ [symbol] ’ in ring E is absent. R³ can also be a carbonyl group that together formed by the connected carbon atom, wherein the alkyl is optionally substituted by one or more halogen or —OH;
each occurrence of R^{g}, R^{h}, Rⁱ is independently selected from H, methyl, cyclopropyl, cyclopentyl or ethyl; preferably methyl;
R⁴ is selected from C1˜C3 alkyl, C3˜C6 cycloalkyl, phenyl, naphthyl, 5- to 6-membered nitrogen-containing heteroaryl or 5- to 6-membered sulphur-containing heteroaryl, wherein the alkyl, cycloalkyl, aryl, nitrogen-containing heteroaryl, sulphur-containing heteroaryl are optionally substituted by 1˜3 R²⁶;
each occurrence of R² is independently selected from halogen, cyano, C1˜C3 alkyl, halogenated C1˜C3 alkyl, —O(C1˜C3 alkyl) or —C(O)OH;
R⁵, R⁶ are independently selected from hydrogen, halogen, C1˜C3 alkyl, 5- to 6-membered heteroaryl, —C(O)R¹⁶, —S(O)₂R¹⁶, —P(O)R¹⁶R¹⁷, —C(O)NR^{b}R^{c}, —C(O)OR^{a} or —OR^{a}, wherein the alkyl, heteroaryl are optionally substituted by one or more R¹⁸;
R¹⁶, R¹⁷ are independently selected from C1˜C3 alkyl;
each occurrence of R^{a}, R^{b}, R^{c} is independently selected from H and C1˜C3 alkyl;
each occurrence of R¹⁸ is independently selected from halogen, C1˜C3 alkyl, —OR^{a} or —NR^{b}R^{c};
each occurrence of R⁷ is independently selected from C1˜C3 alkyl, wherein the alkyl is optionally substituted by one or more substituents independently selected from halogen, cyano or C1˜C3 alkyl; n₁ is selected from 0 or 1;
preferably

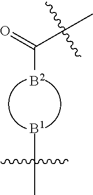

is selected from the following group:

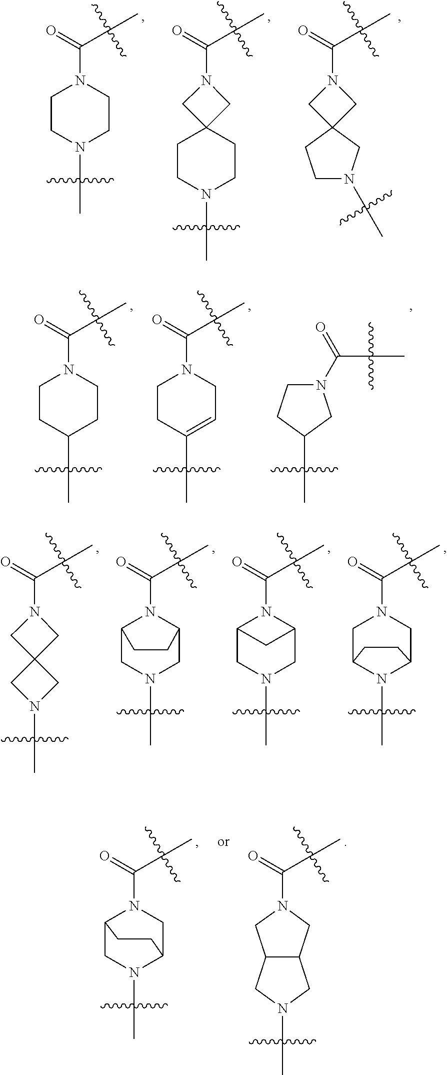

In some embodiments, the compound of the present disclosure has the structure shown in formula VIII or a tautomer, meso isomer, racemate, enantiomer, diastereomer or mixture thereof, or a pharmaceutically acceptable hydrate, solvate or salt thereof:

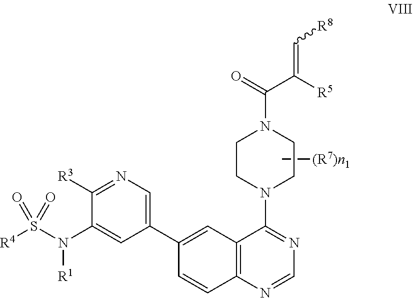

Wherein,
R¹ is H;
R³ is selected from hydrogen, halogen, cyano, C1˜C6 alkyl, —OR^{g}, —C(O)OR^{g}, —SR^{g}, —NR^{h}Rⁱ or wherein the alkyl is optionally substituted by one or more halogen or —OH;
each occurrence of R^{g}, R^{h}, Rⁱ is independently selected from H, methyl, cyclopropyl, cyclopentyl or ethyl; preferably methyl;
R⁴ is selected from C1˜C3 alkyl, cyclohexyl, phenyl, pyridyl, thienyl, naphthyl, pyrrolyl or thiazolyl, wherein alkyl, cyclohexyl, phenyl, pyridyl, thienyl, naphthyl, pyrrolyl, thiazolyl are optionally substituted by 1˜2 R²⁶;
each occurrence of R²⁶ is independently selected from F, Cl, Br, cyano, unsubstituted C1˜C3 alkyl or C1˜C3 alkyl substituted by 1 to 3 halogen, —OR^{j} or —C(O)OR^{j};
R^{j} is selected from H and C1˜C3 alkyl;
R⁵, R⁶ are independently selected from hydrogen, halogen, C1˜C3 alkyl, 5- to 6-membered heteroaryl, —C(O)R¹⁶, —S(O)₂R¹⁶, —P(O)R¹⁶R¹⁷, —C(O)NR^{b}R^{c}, —C(O)OR^{a} or —OR^{a}, wherein the alkyl, heteroaryl am optionally substituted by one or more R¹⁸;
R¹⁶, R¹⁷ is independently selected from C1˜C3 alkyl;
each occurrence of R¹⁸ is independently selected from halogen, C1˜C3 alkyl, —OR^{a} or —NR^{b}R^{c};
each occurrence of R^{a}, R^{b}, R^{c} is independently selected from H and C1˜C3 alkyl;
each occurrence of R⁷ is independently selected from C1˜C3 alkyl, wherein the alkyl is optionally substituted by one or more substituents independently selected from halogen, cyano or C1˜C3 alkyl;
n₁ is selected from 0 or 1.

In some embodiments, the compound of the present disclosure has the structure shown in formula VIII or a tautomer, meso isomer, racemate, enantiomer, diastereomer or mixture thereof, or a pharmaceutically acceptable hydrate, solvate or salt thereof:
wherein,
R¹ is H;
R³ is selected from hydrogen, Cl, —CF₃, —OCH₃, —OCH₂CH₃, —OCF₃, —OCHF₂, —N(CH₃)₂, —CH₃, —CH₂OH, —OCH₂CF₃, —OH, —NHCH₃, —SCH₃, —OCD₃, —CN or —C(O)OCH₃;
R⁴ is selected from methyl, cyclohexyl,

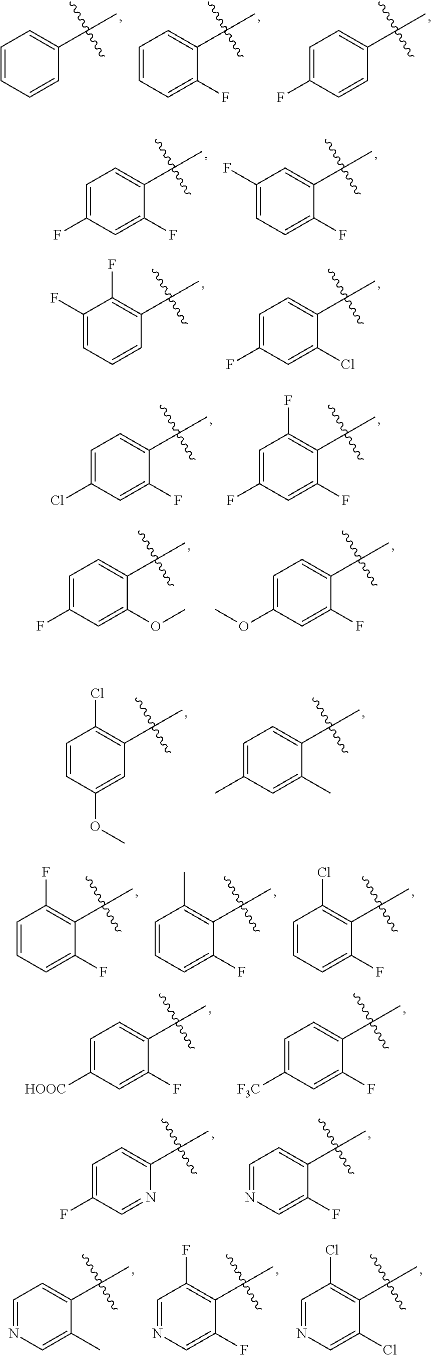

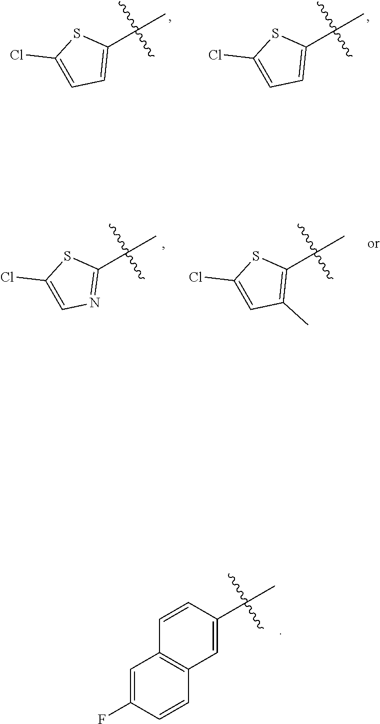

R⁵ is selected from H, F, CN, —CH(CH₃)₂, —CH(OH)CH₃, —CH₂OCH₃, —OCH₃ or —C(O)CH₃;
R⁶ is selected from H, —C(CH₃)₃, —C(O)CH₃, —C(O)CH₂CH₂CH₃, —C(O)NHCH₃, —CH₂N(CH₃)₂, —CH₂F,

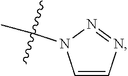 [SMILES: *n1ccnn1]

—CH₂OCH₃, CF₃,

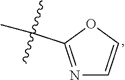 [SMILES: *c1ncco1]

—C(O)OCH₃, —P(O)(CH₃), —S(O)₂CH₃, —C(O)CF₃ or —C(O)CH₂CH₃.
R⁷ is selected from hydrogen, methyl or —CH₂CN;
n₁ is selected from 0 or 1.

In some embodiments of the present disclosure, the compound has the structure shown as followed:

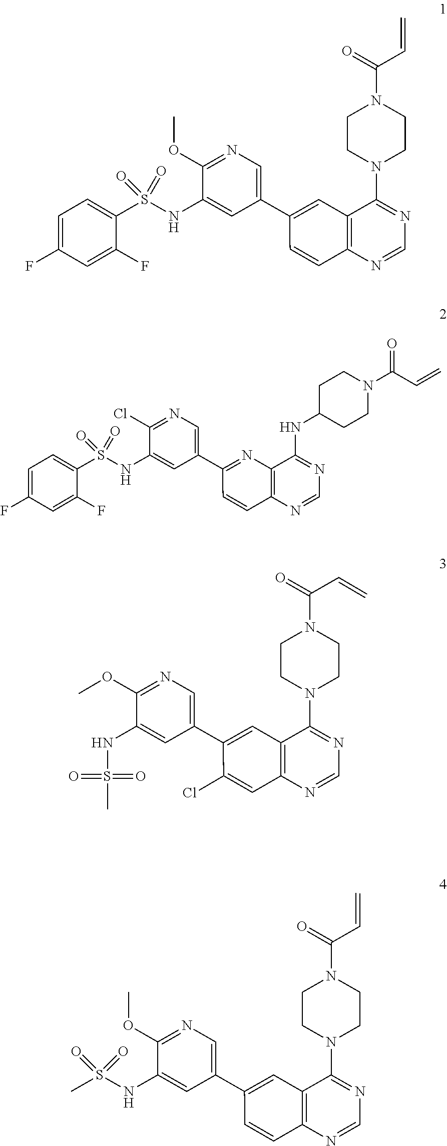

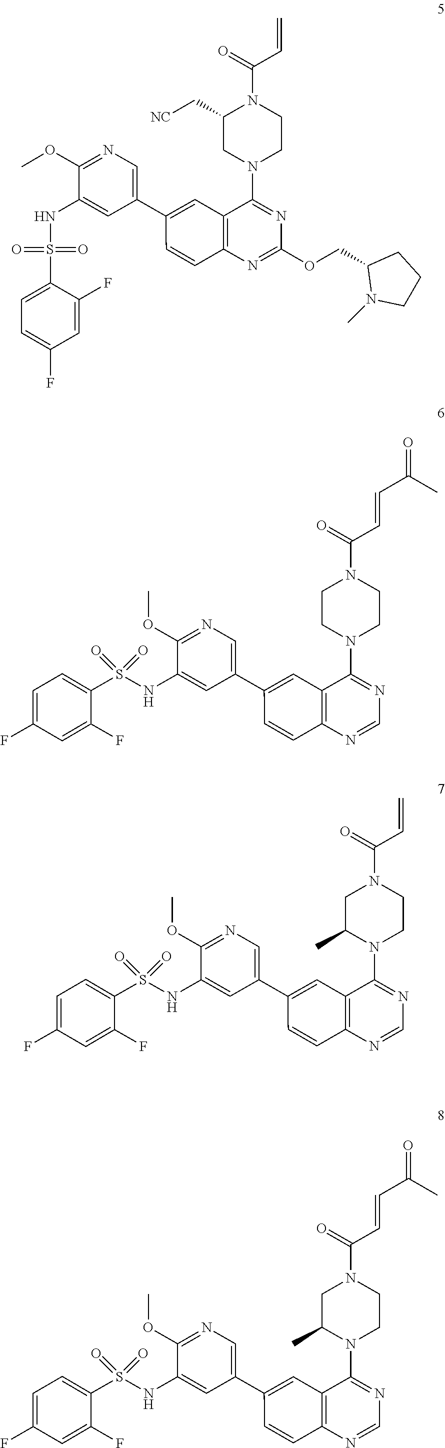 [SMILES: C=CC(=O)N1CCN(c2nc(OC[C@@H]3CCCN3C)nc3ccc(-c4cnc(OC)c(NS(=O)(=O)c5ccc(F)cc5F)c4)cc23)C[C@@H]1CC#N ; COc1ncc(-c2ccc3ncnc(N4CCN(C(=O)/C=C/C(C)=O)CC4)c3c2)cc1NS(=O)(=O)c1ccc(F)cc1F ; C=CC(=O)N1CCN(c2ncnc3ccc(-c4cnc(OC)c(NS(=O)(=O)c5ccc(F)cc5F)c4)cc23)[C@@H](C)C1 ; COc1ncc(-c2ccc3ncnc(N4CCN(C(=O)/C=C/C(C)=O)C[C@@H]4C)c3c2)cc1NS(=O)(=O)c1ccc(F)cc1F]

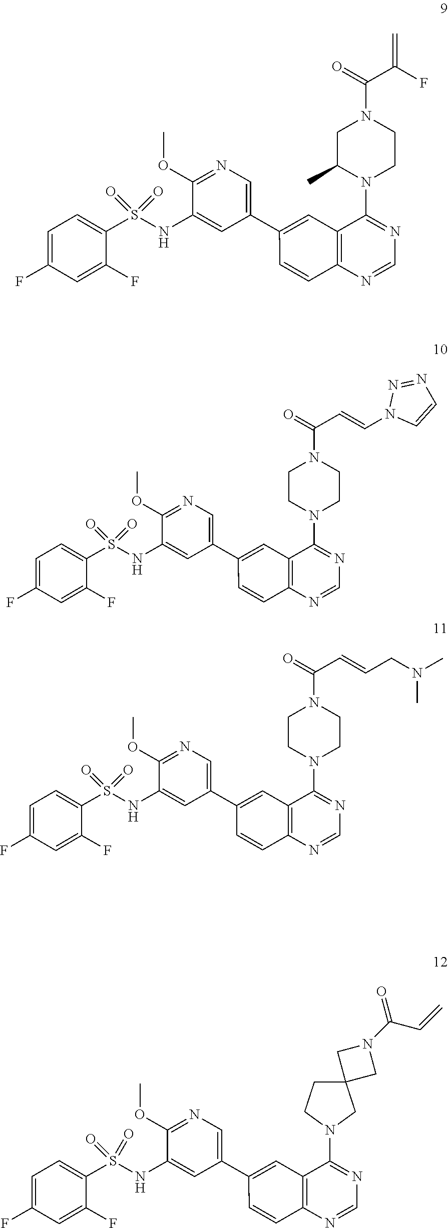 [SMILES: C=C(F)C(=O)N1CCN(c2ncnc3ccc(-c4cnc(OC)c(NS(=O)(=O)c5ccc(F)cc5F)c4)cc23)[C@@H](C)C1 ; COc1ncc(-c2ccc3ncnc(N4CCN(C(=O)/C=C/n5ccnn5)CC4)c3c2)cc1NS(=O)(=O)c1ccc(F)cc1F ; COc1ncc(-c2ccc3ncnc(N4CCN(C(=O)/C=C/CN(C)C)CC4)c3c2)cc1NS(=O)(=O)c1ccc(F)cc1F ; C=CC(=O)N1CC2(CCN(c3ncnc4ccc(-c5cnc(OC)c(NS(=O)(=O)c6ccc(F)cc6F)c5)cc34)C2)C1]

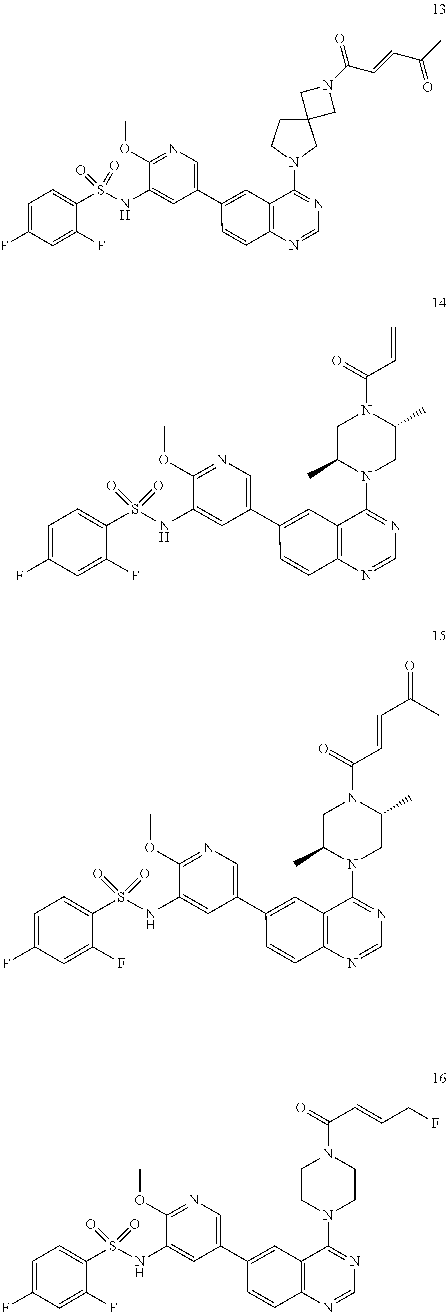 [SMILES: COc1ncc(-c2ccc3ncnc(N4CCC5(CN(C(=O)/C=C/C(C)=O)C5)C4)c3c2)cc1NS(=O)(=O)c1ccc(F)cc1F ; C=CC(=O)N1C[C@H](C)N(c2ncnc3ccc(-c4cnc(OC)c(NS(=O)(=O)c5ccc(F)cc5F)c4)cc23)C[C@H]1C ; COc1ncc(-c2ccc3ncnc(N4C[C@@H](C)N(C(=O)/C=C/C(C)=O)C[C@@H]4C)c3c2)cc1NS(=O)(=O)c1ccc(F)cc1F ; COc1ncc(-c2ccc3ncnc(N4CCN(C(=O)/C=C/CF)CC4)c3c2)cc1NS(=O)(=O)c1ccc(F)cc1F]

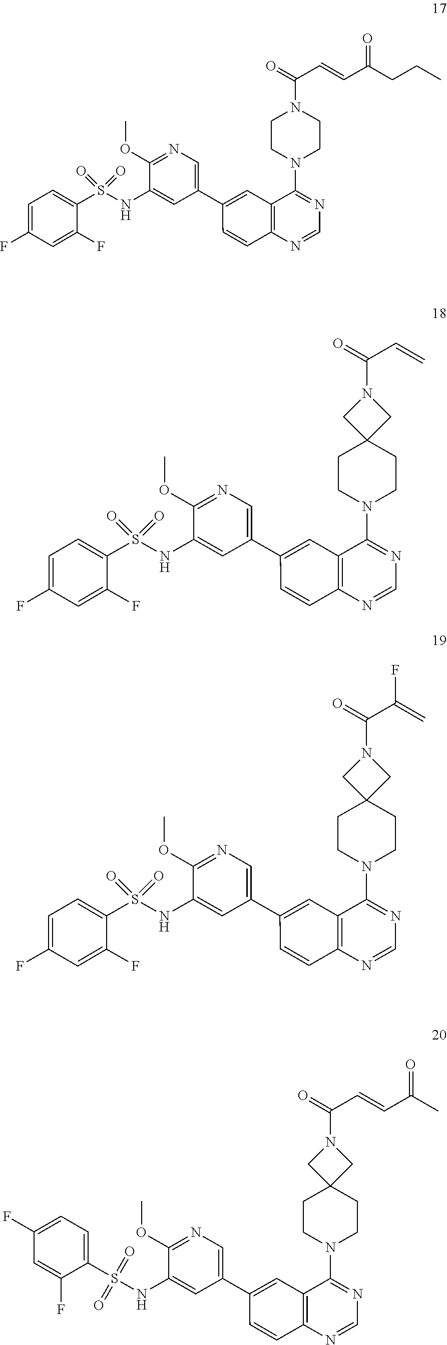 [SMILES: CCCC(=O)/C=C/C(=O)N1CCN(c2ncnc3ccc(-c4cnc(OC)c(NS(=O)(=O)c5ccc(F)cc5F)c4)cc23)CC1 ; C=CC(=O)N1CC2(CCN(c3ncnc4ccc(-c5cnc(OC)c(NS(=O)(=O)c6ccc(F)cc6F)c5)cc34)CC2)C1 ; C=C(F)C(=O)N1CC2(CCN(c3ncnc4ccc(-c5cnc(OC)c(NS(=O)(=O)c6ccc(F)cc6F)c5)cc34)CC2)C1 ; COc1ncc(-c2ccc3ncnc(N4CCC5(CC4)CN(C(=O)/C=C/C(C)=O)C5)c3c2)cc1NS(=O)(=O)c1ccc(F)cc1F]

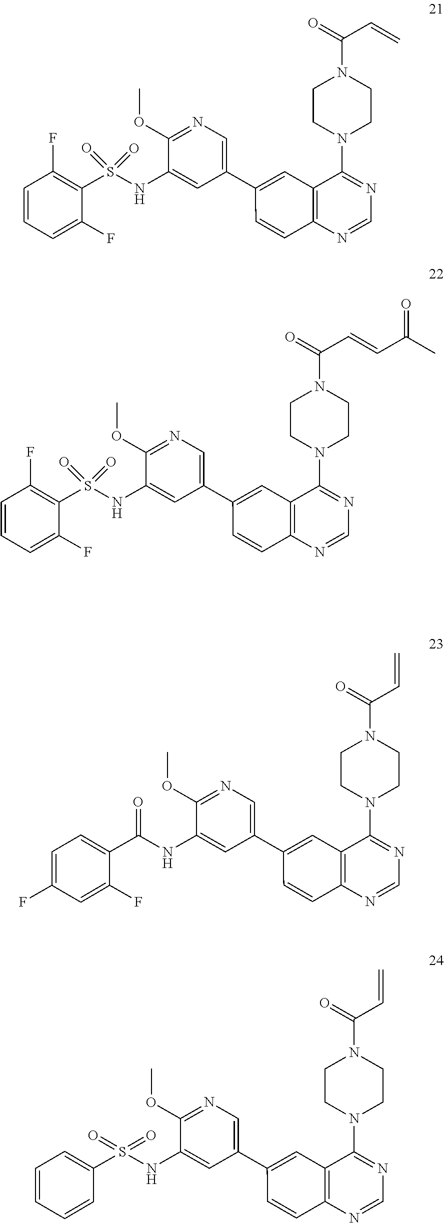 [SMILES: C=CC(=O)N1CCN(c2ncnc3ccc(-c4cnc(OC)c(NS(=O)(=O)c5c(F)cccc5F)c4)cc23)CC1 ; COc1ncc(-c2ccc3ncnc(N4CCN(C(=O)/C=C/C(C)=O)CC4)c3c2)cc1NS(=O)(=O)c1c(F)cccc1F ; C=CC(=O)N1CCN(c2ncnc3ccc(-c4cnc(OC)c(NC(=O)c5ccc(F)cc5F)c4)cc23)CC1 ; C=CC(=O)N1CCN(c2ncnc3ccc(-c4cnc(OC)c(NS(=O)(=O)c5ccccc5)c4)cc23)CC1]

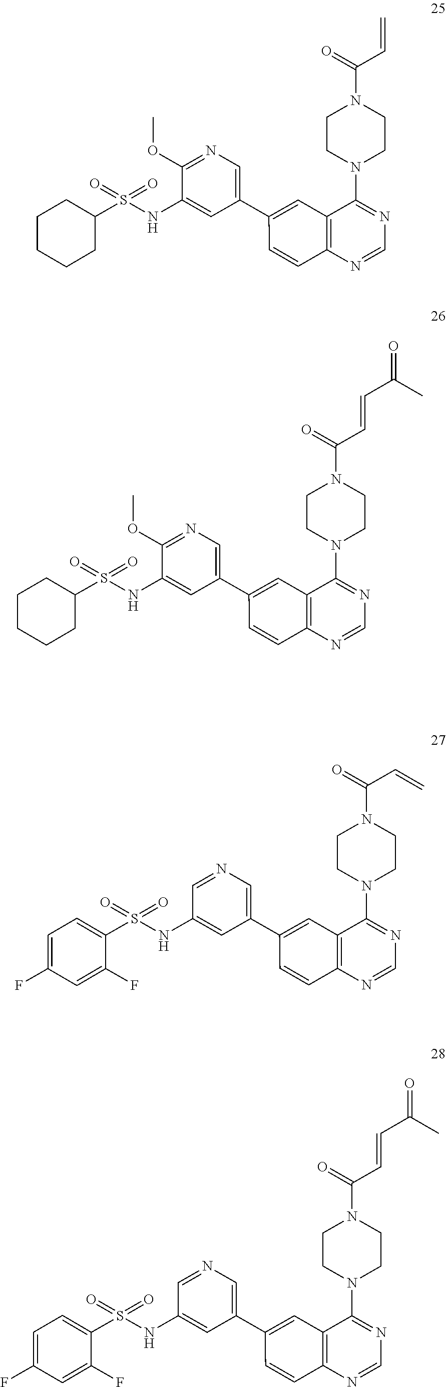 [SMILES: C=CC(=O)N1CCN(c2ncnc3ccc(-c4cnc(OC)c(NS(=O)(=O)C5CCCCC5)c4)cc23)CC1 ; COc1ncc(-c2ccc3ncnc(N4CCN(C(=O)/C=C/C(C)=O)CC4)c3c2)cc1NS(=O)(=O)C1CCCCC1 ; C=CC(=O)N1CCN(c2ncnc3ccc(-c4cncc(NS(=O)(=O)c5ccc(F)cc5F)c4)cc23)CC1 ; CC(=O)/C=C/C(=O)N1CCN(c2ncnc3ccc(-c4cncc(NS(=O)(=O)c5ccc(F)cc5F)c4)cc23)CC1]

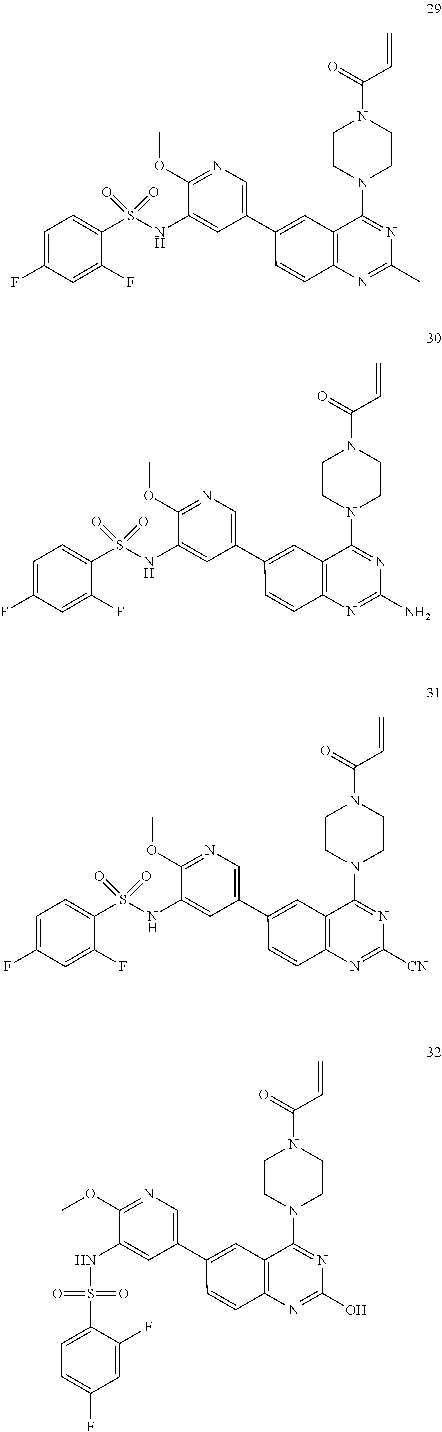 [SMILES: C=CC(=O)N1CCN(c2nc(C)nc3ccc(-c4cnc(OC)c(NS(=O)(=O)c5ccc(F)cc5F)c4)cc23)CC1 ; C=CC(=O)N1CCN(c2nc(N)nc3ccc(-c4cnc(OC)c(NS(=O)(=O)c5ccc(F)cc5F)c4)cc23)CC1 ; C=CC(=O)N1CCN(c2nc(C#N)nc3ccc(-c4cnc(OC)c(NS(=O)(=O)c5ccc(F)cc5F)c4)cc23)CC1 ; C=CC(=O)N1CCN(c2nc(O)nc3ccc(-c4cnc(OC)c(NS(=O)(=O)c5ccc(F)cc5F)c4)cc23)CC1]

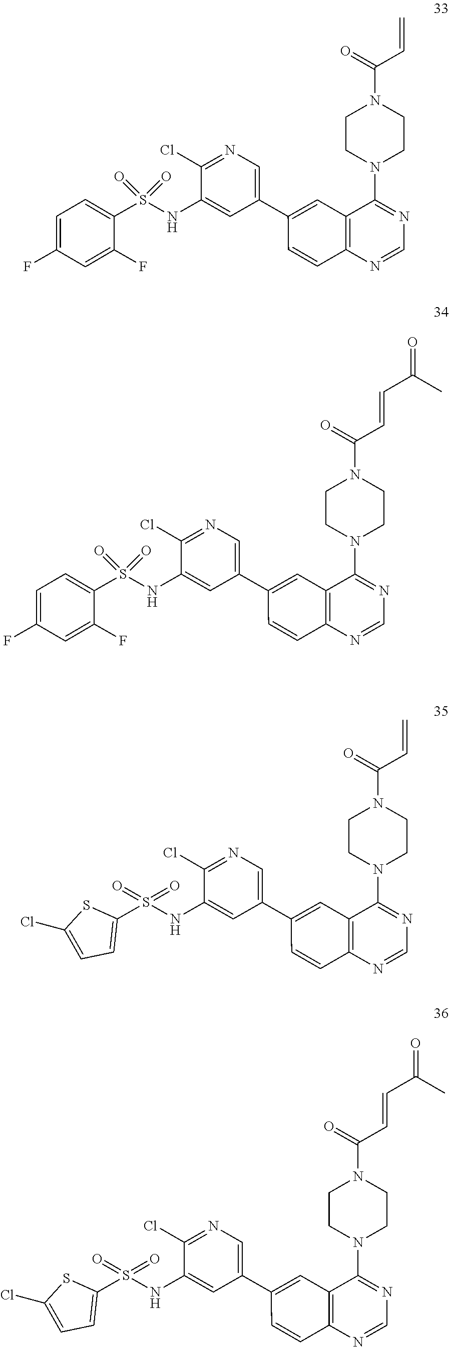 [SMILES: C=CC(=O)N1CCN(c2ncnc3ccc(-c4cnc(Cl)c(NS(=O)(=O)c5ccc(F)cc5F)c4)cc23)CC1 ; CC(=O)/C=C/C(=O)N1CCN(c2ncnc3ccc(-c4cnc(Cl)c(NS(=O)(=O)c5ccc(F)cc5F)c4)cc23)CC1 ; C=CC(=O)N1CCN(c2ncnc3ccc(-c4cnc(Cl)c(NS(=O)(=O)c5ccc(Cl)s5)c4)cc23)CC1 ; CC(=O)/C=C/C(=O)N1CCN(c2ncnc3ccc(-c4cnc(Cl)c(NS(=O)(=O)c5ccc(Cl)s5)c4)cc23)CC1]

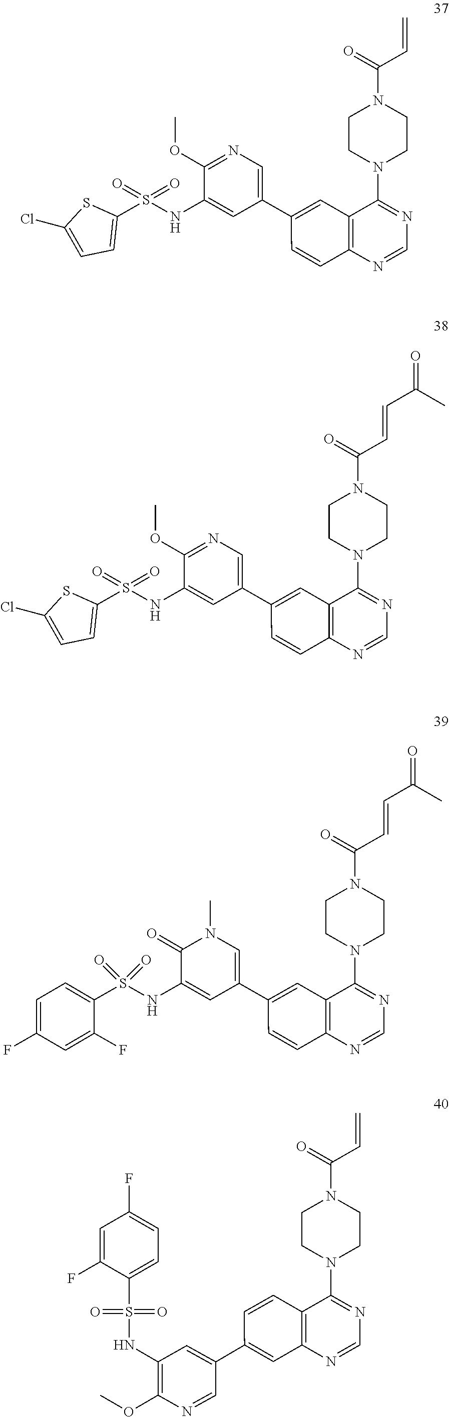 [SMILES: C=CC(=O)N1CCN(c2ncnc3ccc(-c4cnc(OC)c(NS(=O)(=O)c5ccc(Cl)s5)c4)cc23)CC1 ; COc1ncc(-c2ccc3ncnc(N4CCN(C(=O)/C=C/C(C)=O)CC4)c3c2)cc1NS(=O)(=O)c1ccc(Cl)s1 ; CC(=O)/C=C/C(=O)N1CCN(c2ncnc3ccc(-c4cc(NS(=O)(=O)c5ccc(F)cc5F)c(=O)n(C)c4)cc23)CC1 ; C=CC(=O)N1CCN(c2ncnc3cc(-c4cnc(OC)c(NS(=O)(=O)c5ccc(F)cc5F)c4)ccc23)CC1]

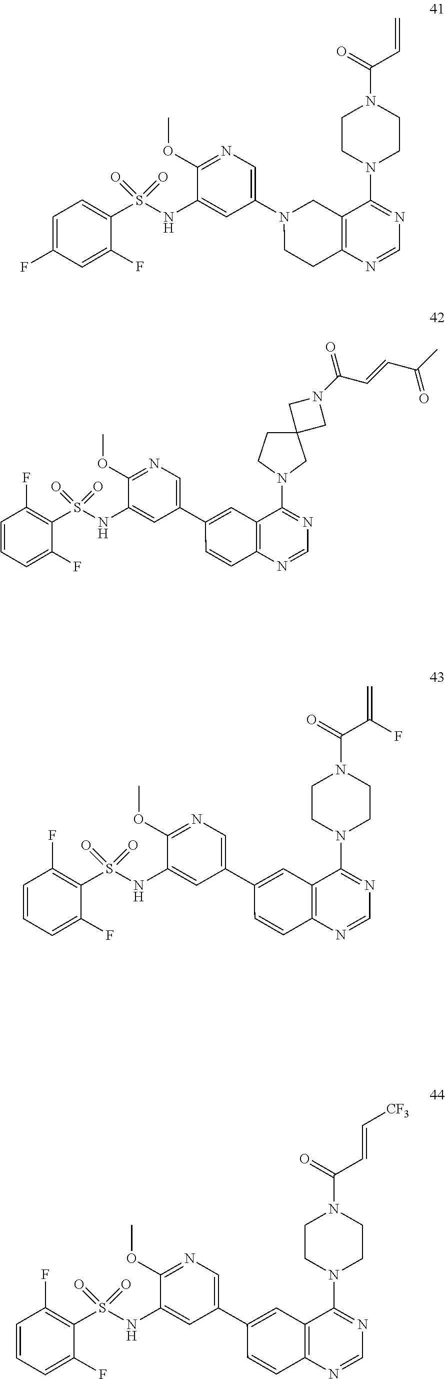 [SMILES: C=CC(=O)N1CCN(c2ncnc3c2CN(c2cnc(OC)c(NS(=O)(=O)c4ccc(F)cc4F)c2)CC3)CC1 ; COc1ncc(-c2ccc3ncnc(N4CCC5(CN(C(=O)/C=C/C(C)=O)C5)C4)c3c2)cc1NS(=O)(=O)c1c(F)cccc1F ; C=C(F)C(=O)N1CCN(c2ncnc3ccc(-c4cnc(OC)c(NS(=O)(=O)c5c(F)cccc5F)c4)cc23)CC1 ; COc1ncc(-c2ccc3ncnc(N4CCN(C(=O)/C=C/C(F)(F)F)CC4)c3c2)cc1NS(=O)(=O)c1c(F)cccc1F]

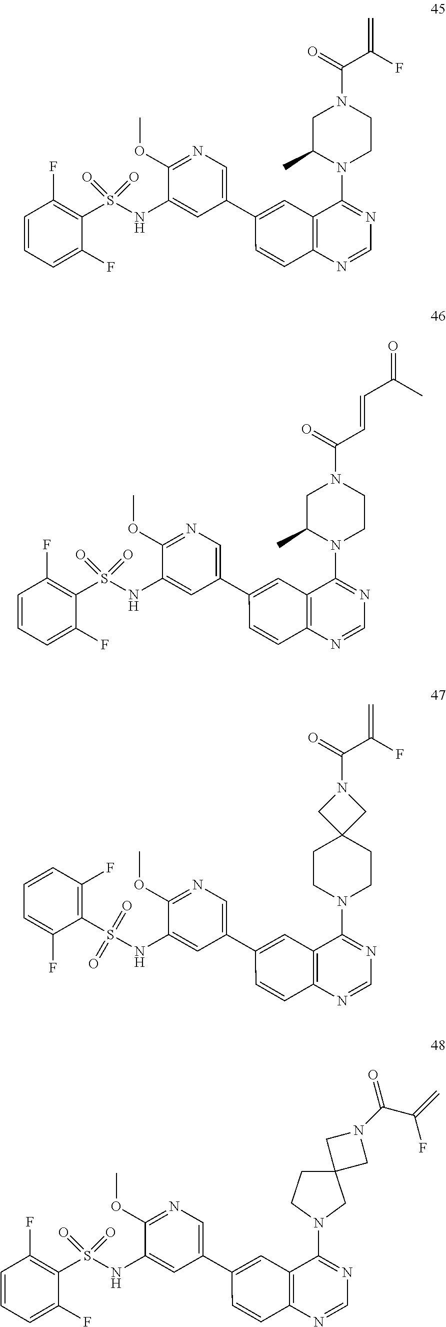 [SMILES: C=C(F)C(=O)N1CCN(c2ncnc3ccc(-c4cnc(OC)c(NS(=O)(=O)c5c(F)cccc5F)c4)cc23)[C@@H](C)C1 ; COc1ncc(-c2ccc3ncnc(N4CCN(C(=O)/C=C/C(C)=O)C[C@@H]4C)c3c2)cc1NS(=O)(=O)c1c(F)cccc1F ; C=C(F)C(=O)N1CC2(CCN(c3ncnc4ccc(-c5cnc(OC)c(NS(=O)(=O)c6c(F)cccc6F)c5)cc34)CC2)C1 ; C=C(F)C(=O)N1CC2(CCN(c3ncnc4ccc(-c5cnc(OC)c(NS(=O)(=O)c6c(F)cccc6F)c5)cc34)C2)C1]

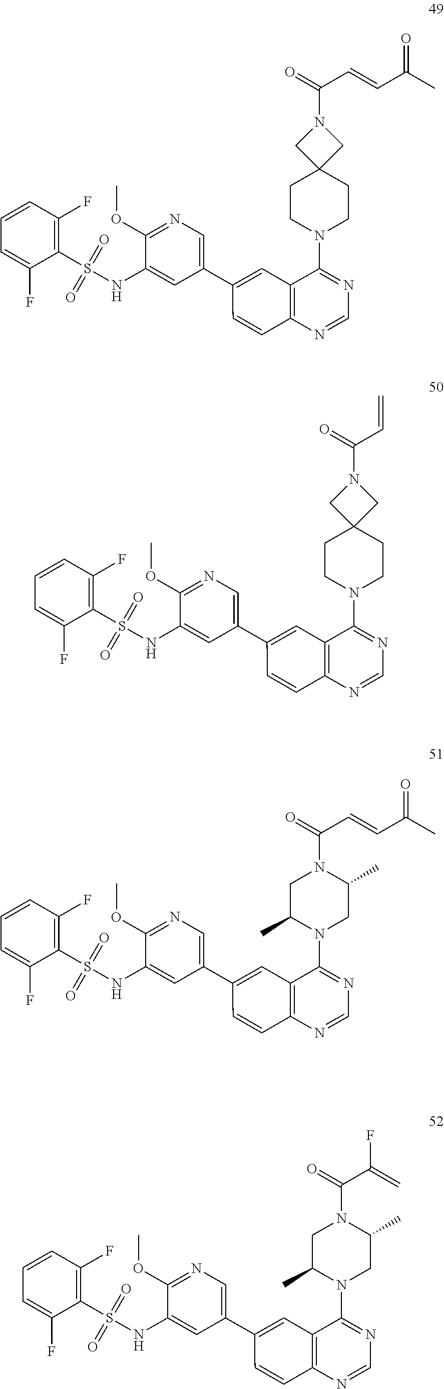 [SMILES: COc1ncc(-c2ccc3ncnc(N4CCC5(CC4)CN(C(=O)/C=C/C(C)=O)C5)c3c2)cc1NS(=O)(=O)c1c(F)cccc1F ; C=CC(=O)N1CC2(CCN(c3ncnc4ccc(-c5cnc(OC)c(NS(=O)(=O)c6c(F)cccc6F)c5)cc34)CC2)C1 ; COc1ncc(-c2ccc3ncnc(N4C[C@@H](C)N(C(=O)/C=C/C(C)=O)C[C@@H]4C)c3c2)cc1NS(=O)(=O)c1c(F)cccc1F ; C=C(F)C(=O)N1C[C@H](C)N(c2ncnc3ccc(-c4cnc(OC)c(NS(=O)(=O)c5c(F)cccc5F)c4)cc23)C[C@H]1C]

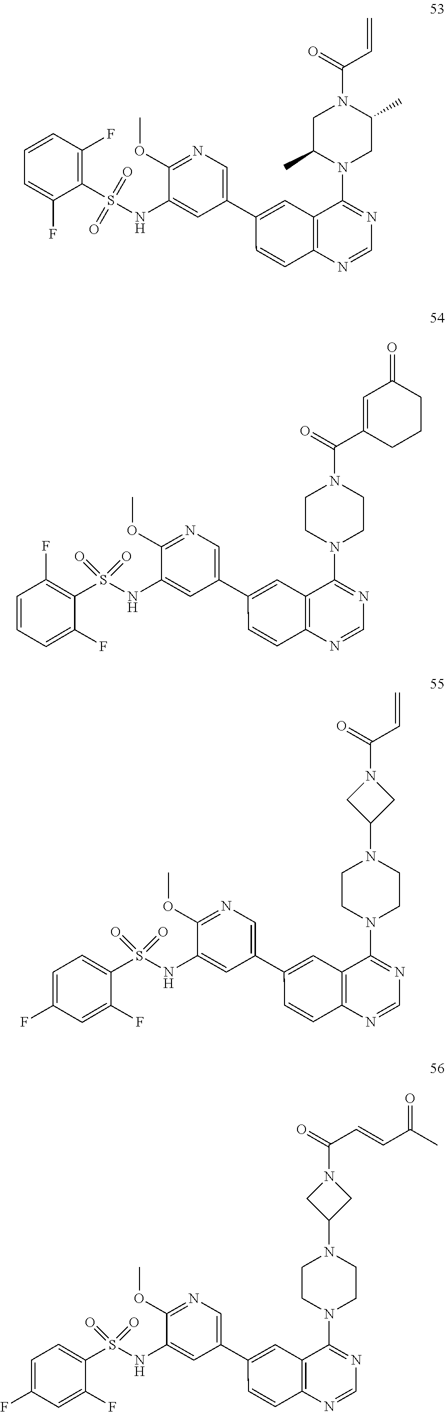 [SMILES: C=CC(=O)N1C[C@H](C)N(c2ncnc3ccc(-c4cnc(OC)c(NS(=O)(=O)c5c(F)cccc5F)c4)cc23)C[C@H]1C ; COc1ncc(-c2ccc3ncnc(N4CCN(C(=O)C5=CC(=O)CCC5)CC4)c3c2)cc1NS(=O)(=O)c1c(F)cccc1F ; C=CC(=O)N1CC(N2CCN(c3ncnc4ccc(-c5cnc(OC)c(NS(=O)(=O)c6ccc(F)cc6F)c5)cc34)CC2)C1 ; COc1ncc(-c2ccc3ncnc(N4CCN(C5CN(C(=O)/C=C/C(C)=O)C5)CC4)c3c2)cc1NS(=O)(=O)c1ccc(F)cc1F]

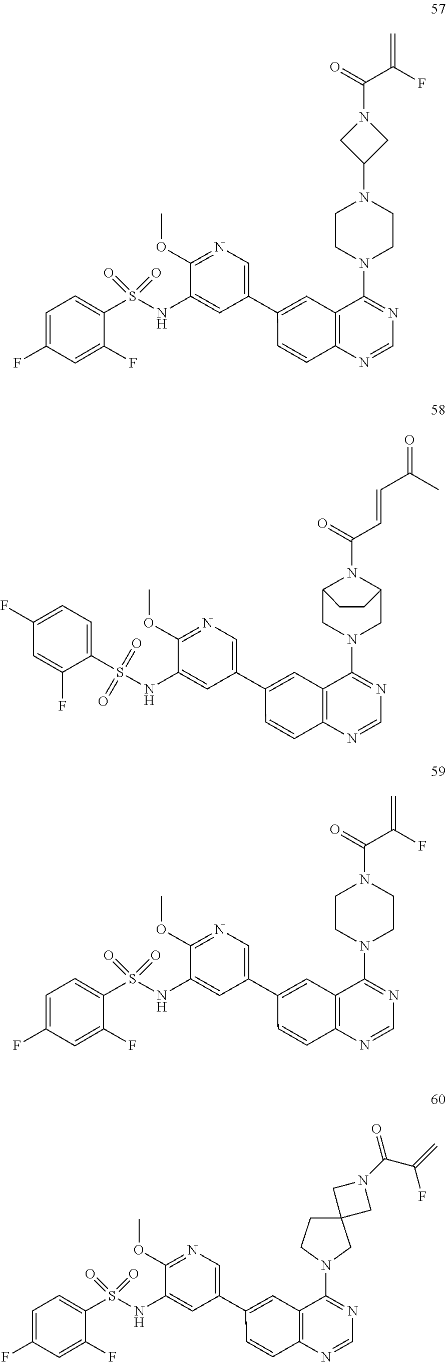 [SMILES: C=C(F)C(=O)N1CC(N2CCN(c3ncnc4ccc(-c5cnc(OC)c(NS(=O)(=O)c6ccc(F)cc6F)c5)cc34)CC2)C1 ; COc1ncc(-c2ccc3ncnc(N4CC5CCC(C4)N5C(=O)/C=C/C(C)=O)c3c2)cc1NS(=O)(=O)c1ccc(F)cc1F ; C=C(F)C(=O)N1CCN(c2ncnc3ccc(-c4cnc(OC)c(NS(=O)(=O)c5ccc(F)cc5F)c4)cc23)CC1 ; C=C(F)C(=O)N1CC2(CCN(c3ncnc4ccc(-c5cnc(OC)c(NS(=O)(=O)c6ccc(F)cc6F)c5)cc34)C2)C1]

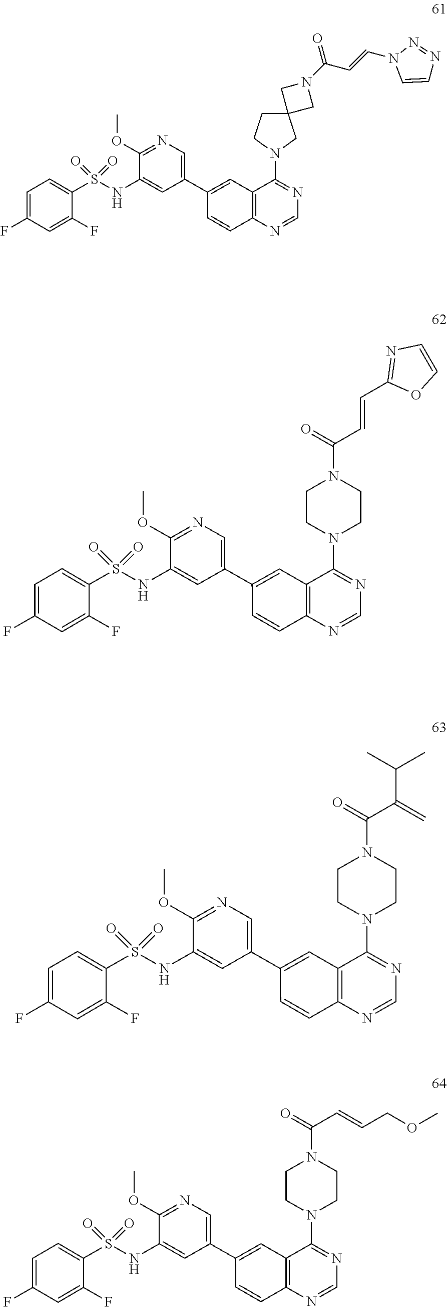 [SMILES: COc1ncc(-c2ccc3ncnc(N4CCC5(CN(C(=O)/C=C/n6ccnn6)C5)C4)c3c2)cc1NS(=O)(=O)c1ccc(F)cc1F ; COc1ncc(-c2ccc3ncnc(N4CCN(C(=O)/C=C/c5ncco5)CC4)c3c2)cc1NS(=O)(=O)c1ccc(F)cc1F ; C=C(C(=O)N1CCN(c2ncnc3ccc(-c4cnc(OC)c(NS(=O)(=O)c5ccc(F)cc5F)c4)cc23)CC1)C(C)C ; COC/C=C/C(=O)N1CCN(c2ncnc3ccc(-c4cnc(OC)c(NS(=O)(=O)c5ccc(F)cc5F)c4)cc23)CC1]

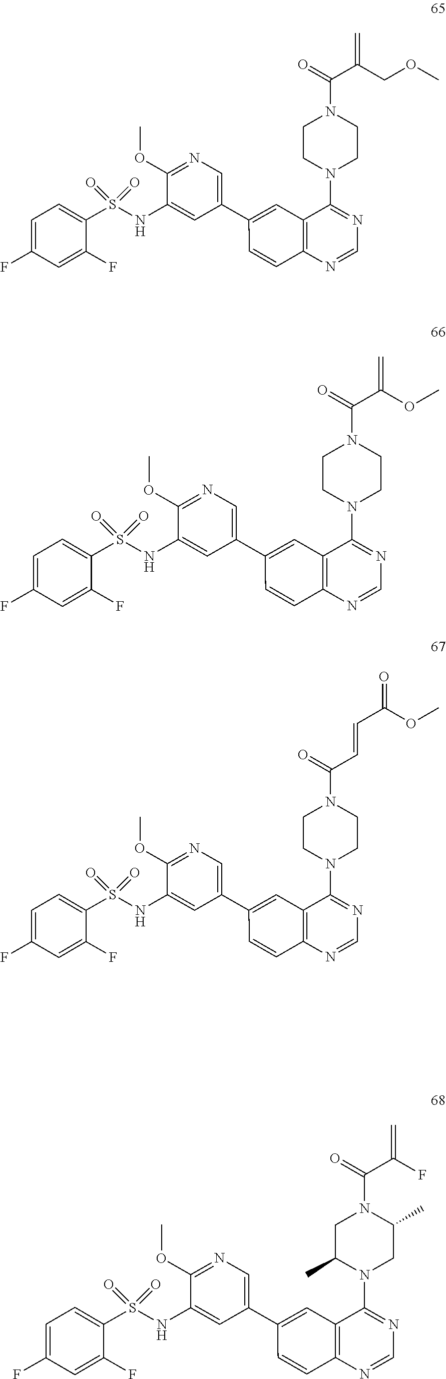 [SMILES: C=C(COC)C(=O)N1CCN(c2ncnc3ccc(-c4cnc(OC)c(NS(=O)(=O)c5ccc(F)cc5F)c4)cc23)CC1 ; C=C(OC)C(=O)N1CCN(c2ncnc3ccc(-c4cnc(OC)c(NS(=O)(=O)c5ccc(F)cc5F)c4)cc23)CC1 ; COC(=O)/C=C/C(=O)N1CCN(c2ncnc3ccc(-c4cnc(OC)c(NS(=O)(=O)c5ccc(F)cc5F)c4)cc23)CC1 ; C=C(F)C(=O)N1C[C@H](C)N(c2ncnc3ccc(-c4cnc(OC)c(NS(=O)(=O)c5ccc(F)cc5F)c4)cc23)C[C@H]1C]

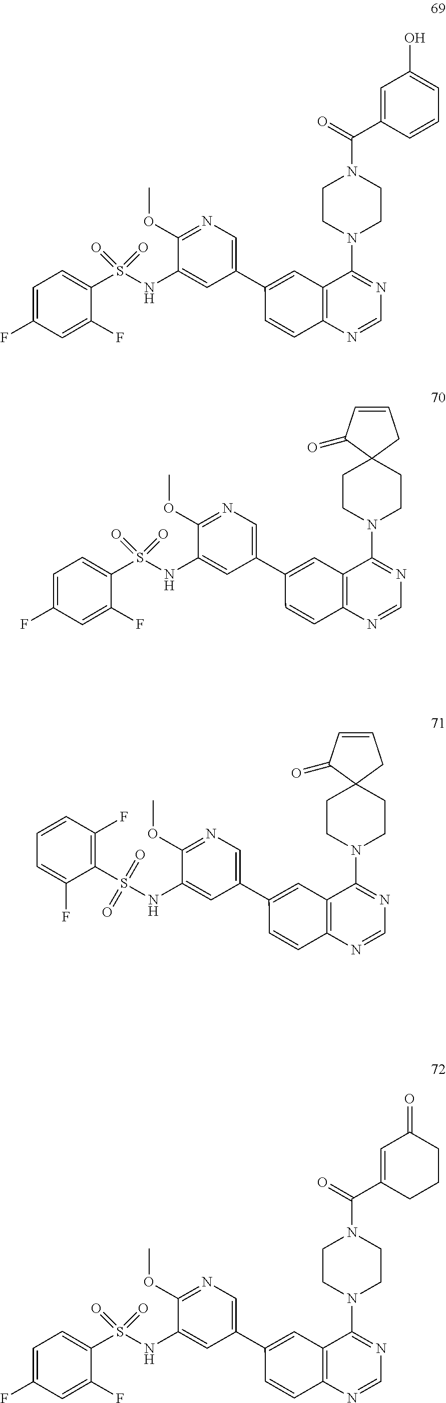 [SMILES: COc1ncc(-c2ccc3ncnc(N4CCN(C(=O)c5cccc(O)c5)CC4)c3c2)cc1NS(=O)(=O)c1ccc(F)cc1F ; COc1ncc(-c2ccc3ncnc(N4CCC5(CC=CC5=O)CC4)c3c2)cc1NS(=O)(=O)c1ccc(F)cc1F ; COc1ncc(-c2ccc3ncnc(N4CCC5(CC=CC5=O)CC4)c3c2)cc1NS(=O)(=O)c1c(F)cccc1F ; COc1ncc(-c2ccc3ncnc(N4CCN(C(=O)C5=CC(=O)CCC5)CC4)c3c2)cc1NS(=O)(=O)c1ccc(F)cc1F]

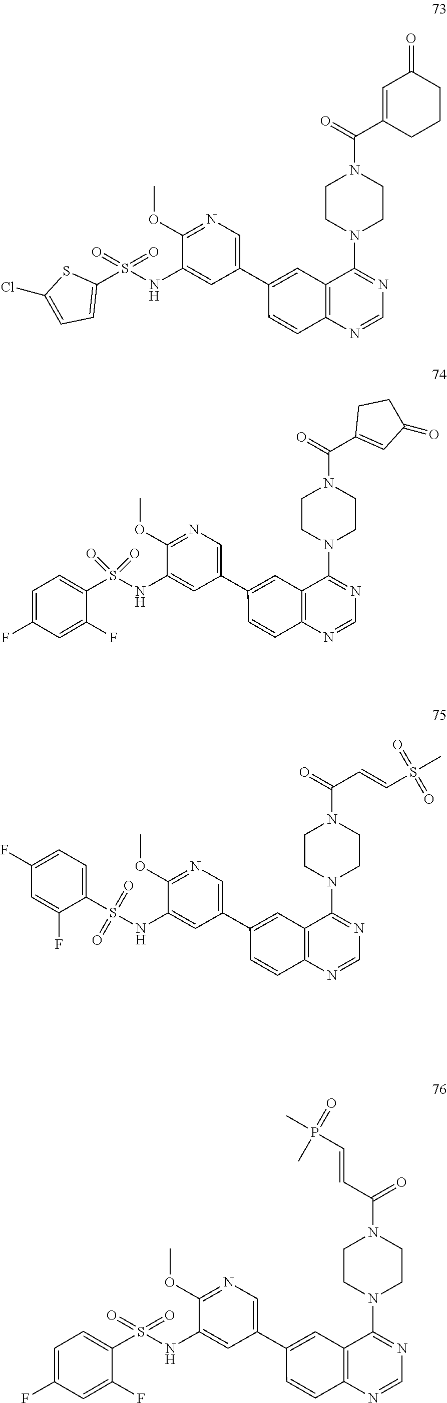 [SMILES: COc1ncc(-c2ccc3ncnc(N4CCN(C(=O)C5=CC(=O)CCC5)CC4)c3c2)cc1NS(=O)(=O)c1ccc(Cl)s1 ; COc1ncc(-c2ccc3ncnc(N4CCN(C(=O)C5=CC(=O)CC5)CC4)c3c2)cc1NS(=O)(=O)c1ccc(F)cc1F ; COc1ncc(-c2ccc3ncnc(N4CCN(C(=O)/C=C/S(C)(=O)=O)CC4)c3c2)cc1NS(=O)(=O)c1ccc(F)cc1F ; COc1ncc(-c2ccc3ncnc(N4CCN(C(=O)/C=C/P(C)(C)=O)CC4)c3c2)cc1NS(=O)(=O)c1ccc(F)cc1F]

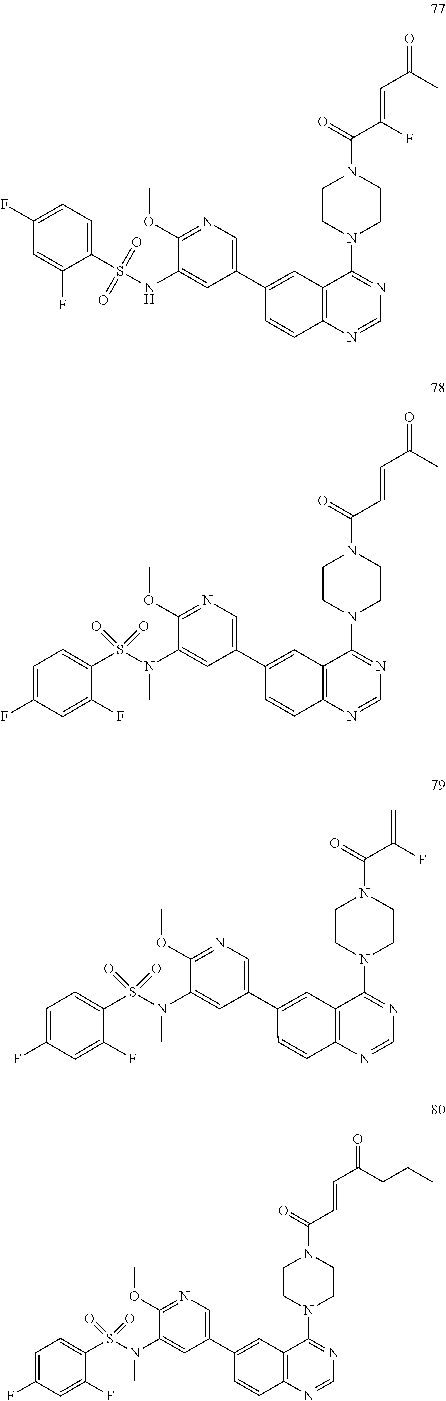 [SMILES: COc1ncc(-c2ccc3ncnc(N4CCN(C(=O)/C(F)=C/C(C)=O)CC4)c3c2)cc1NS(=O)(=O)c1ccc(F)cc1F ; COc1ncc(-c2ccc3ncnc(N4CCN(C(=O)/C=C/C(C)=O)CC4)c3c2)cc1N(C)S(=O)(=O)c1ccc(F)cc1F ; C=C(F)C(=O)N1CCN(c2ncnc3ccc(-c4cnc(OC)c(N(C)S(=O)(=O)c5ccc(F)cc5F)c4)cc23)CC1 ; CCCC(=O)/C=C/C(=O)N1CCN(c2ncnc3ccc(-c4cnc(OC)c(N(C)S(=O)(=O)c5ccc(F)cc5F)c4)cc23)CC1]

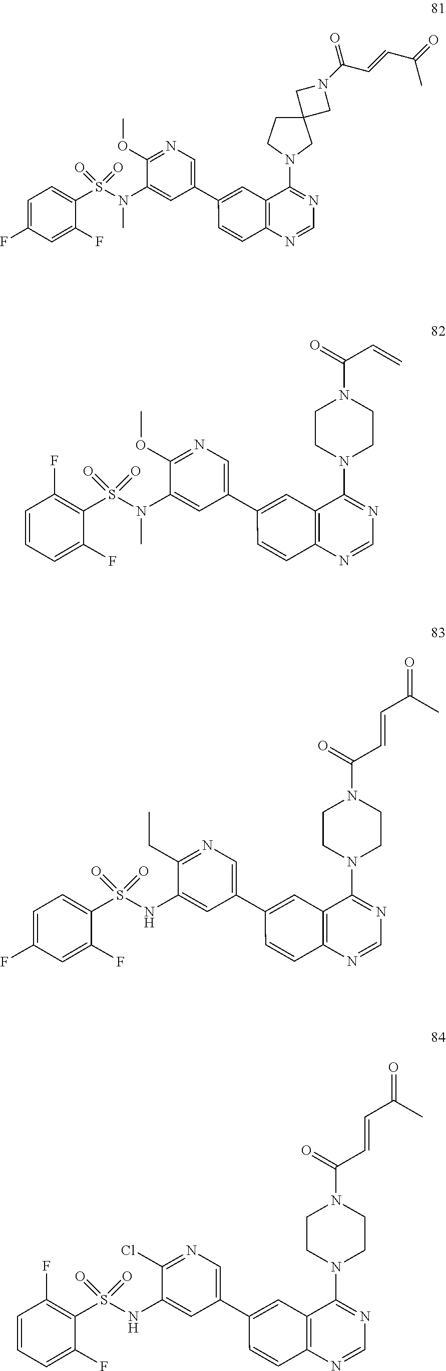 [SMILES: COc1ncc(-c2ccc3ncnc(N4CCC5(CN(C(=O)/C=C/C(C)=O)C5)C4)c3c2)cc1N(C)S(=O)(=O)c1ccc(F)cc1F ; C=CC(=O)N1CCN(c2ncnc3ccc(-c4cnc(OC)c(N(C)S(=O)(=O)c5c(F)cccc5F)c4)cc23)CC1 ; CCc1ncc(-c2ccc3ncnc(N4CCN(C(=O)/C=C/C(C)=O)CC4)c3c2)cc1NS(=O)(=O)c1ccc(F)cc1F ; CC(=O)/C=C/C(=O)N1CCN(c2ncnc3ccc(-c4cnc(Cl)c(NS(=O)(=O)c5c(F)cccc5F)c4)cc23)CC1]

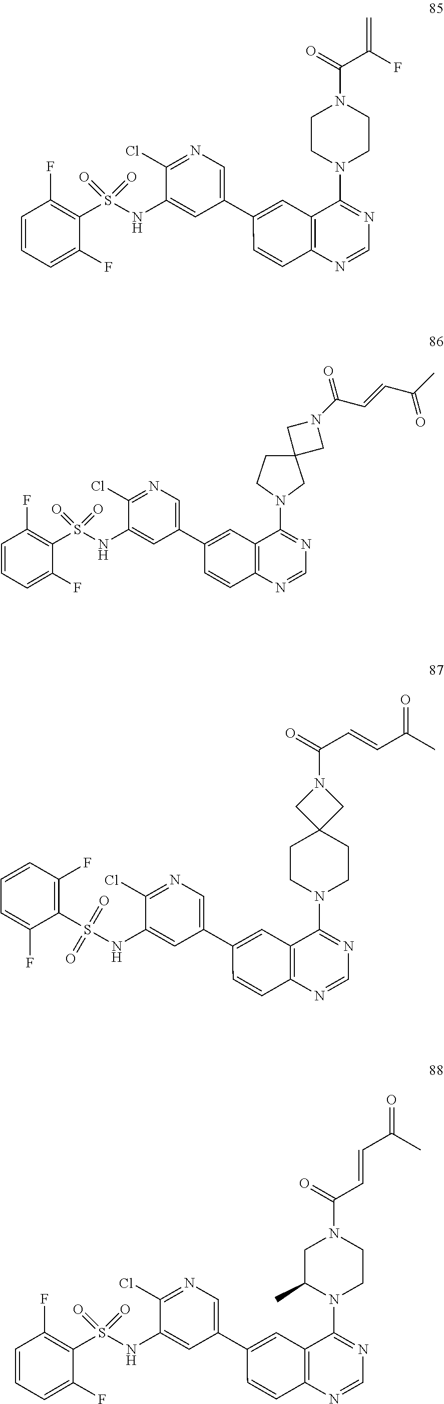 [SMILES: C=C(F)C(=O)N1CCN(c2ncnc3ccc(-c4cnc(Cl)c(NS(=O)(=O)c5c(F)cccc5F)c4)cc23)CC1 ; CC(=O)/C=C/C(=O)N1CC2(CCN(c3ncnc4ccc(-c5cnc(Cl)c(NS(=O)(=O)c6c(F)cccc6F)c5)cc34)C2)C1 ; CC(=O)/C=C/C(=O)N1CC2(CCN(c3ncnc4ccc(-c5cnc(Cl)c(NS(=O)(=O)c6c(F)cccc6F)c5)cc34)CC2)C1 ; CC(=O)/C=C/C(=O)N1CCN(c2ncnc3ccc(-c4cnc(Cl)c(NS(=O)(=O)c5c(F)cccc5F)c4)cc23)[C@@H](C)C1]

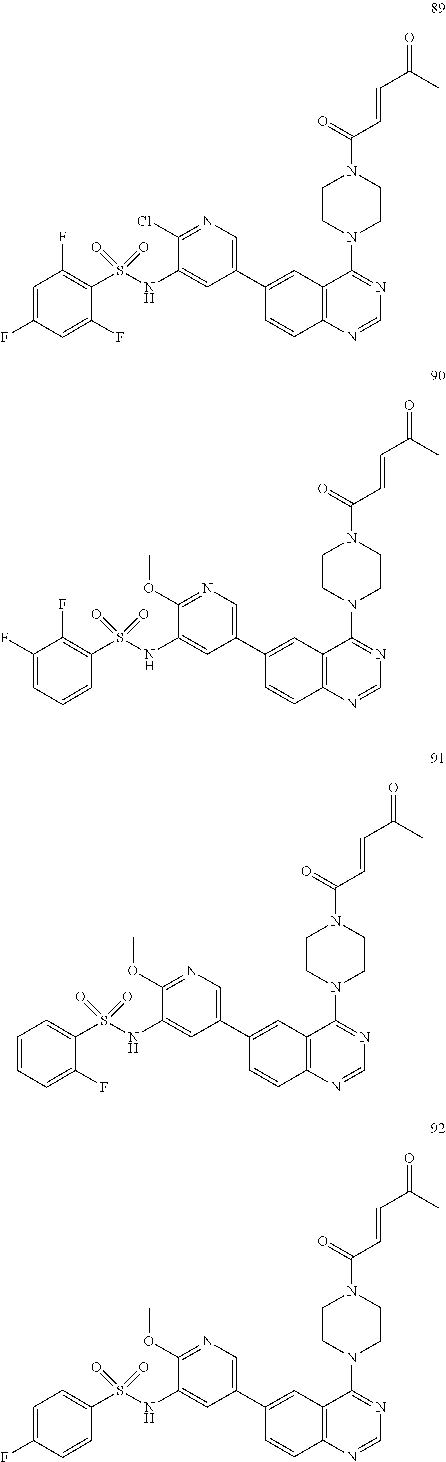 [SMILES: CC(=O)/C=C/C(=O)N1CCN(c2ncnc3ccc(-c4cnc(Cl)c(NS(=O)(=O)c5c(F)cc(F)cc5F)c4)cc23)CC1 ; COc1ncc(-c2ccc3ncnc(N4CCN(C(=O)/C=C/C(C)=O)CC4)c3c2)cc1NS(=O)(=O)c1cccc(F)c1F ; COc1ncc(-c2ccc3ncnc(N4CCN(C(=O)/C=C/C(C)=O)CC4)c3c2)cc1NS(=O)(=O)c1ccccc1F ; COc1ncc(-c2ccc3ncnc(N4CCN(C(=O)/C=C/C(C)=O)CC4)c3c2)cc1NS(=O)(=O)c1ccc(F)cc1]

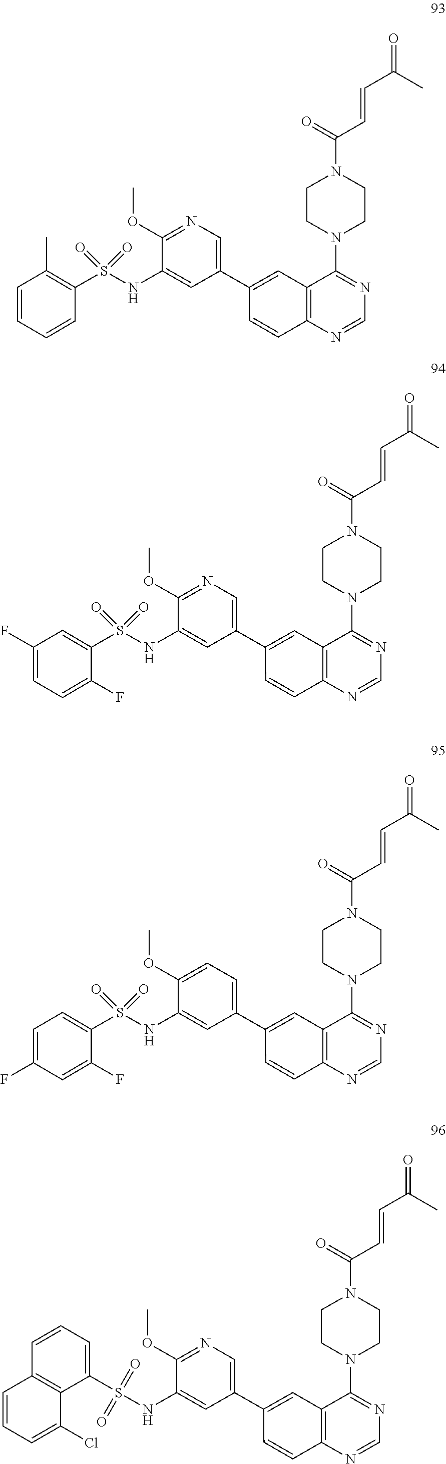 [SMILES: COc1ncc(-c2ccc3ncnc(N4CCN(C(=O)/C=C/C(C)=O)CC4)c3c2)cc1NS(=O)(=O)c1ccccc1C ; COc1ncc(-c2ccc3ncnc(N4CCN(C(=O)/C=C/C(C)=O)CC4)c3c2)cc1NS(=O)(=O)c1cc(F)ccc1F ; COc1ccc(-c2ccc3ncnc(N4CCN(C(=O)/C=C/C(C)=O)CC4)c3c2)cc1NS(=O)(=O)c1ccc(F)cc1F ; COc1ncc(-c2ccc3ncnc(N4CCN(C(=O)/C=C/C(C)=O)CC4)c3c2)cc1NS(=O)(=O)c1cccc2cccc(Cl)c12]

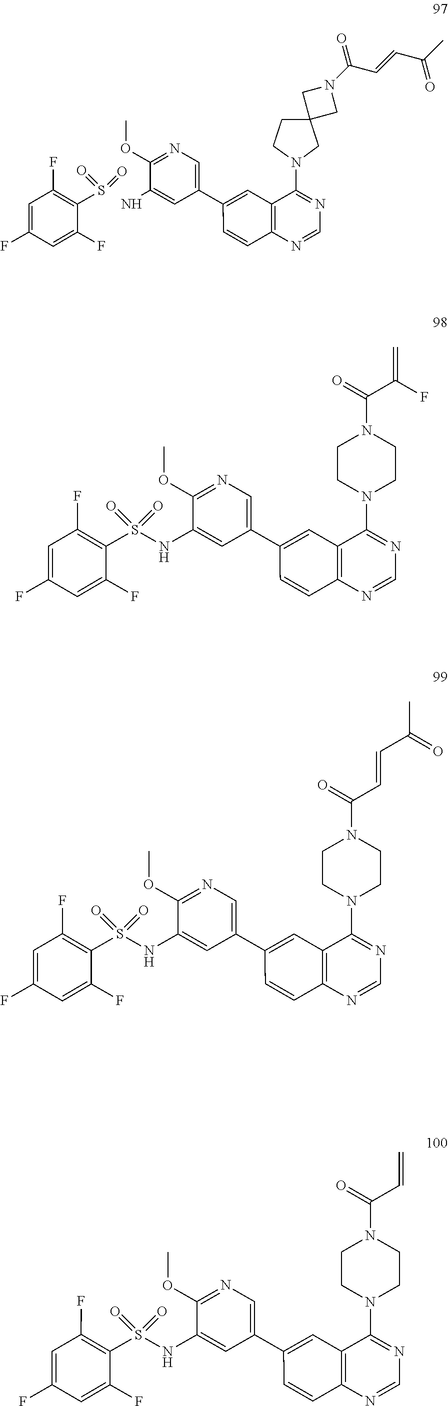

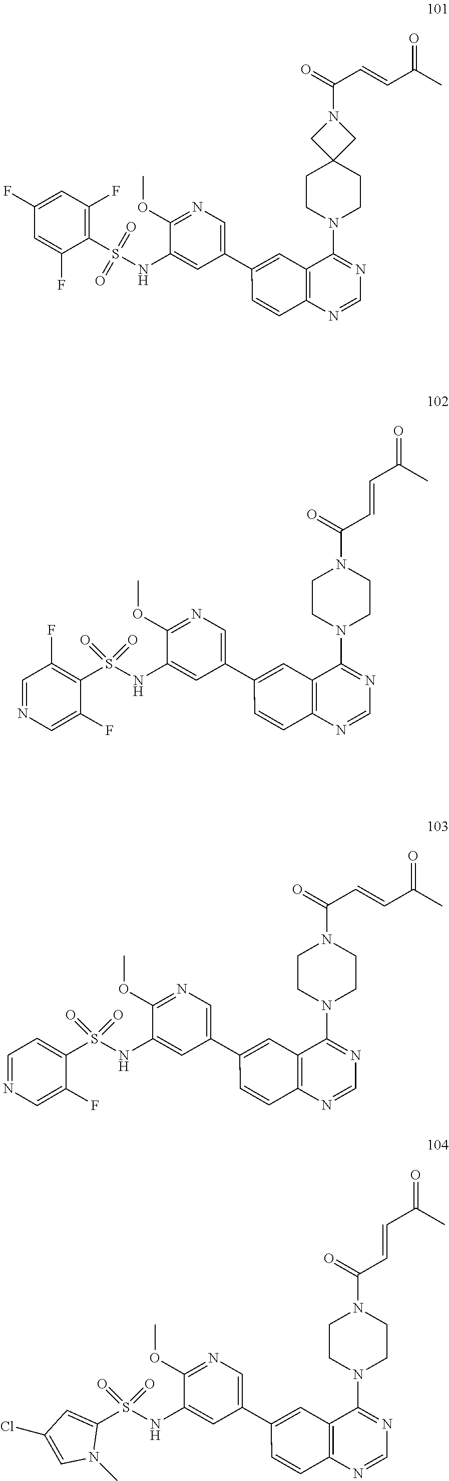 [SMILES: COc1ncc(-c2ccc3ncnc(N4CCC5(CC4)CN(C(=O)/C=C/C(C)=O)C5)c3c2)cc1NS(=O)(=O)c1c(F)cc(F)cc1F ; COc1ncc(-c2ccc3ncnc(N4CCN(C(=O)/C=C/C(C)=O)CC4)c3c2)cc1NS(=O)(=O)c1c(F)cncc1F ; COc1ncc(-c2ccc3ncnc(N4CCN(C(=O)/C=C/C(C)=O)CC4)c3c2)cc1NS(=O)(=O)c1ccncc1F ; COc1ncc(-c2ccc3ncnc(N4CCN(C(=O)/C=C/C(C)=O)CC4)c3c2)cc1NS(=O)(=O)c1cc(Cl)cn1C]

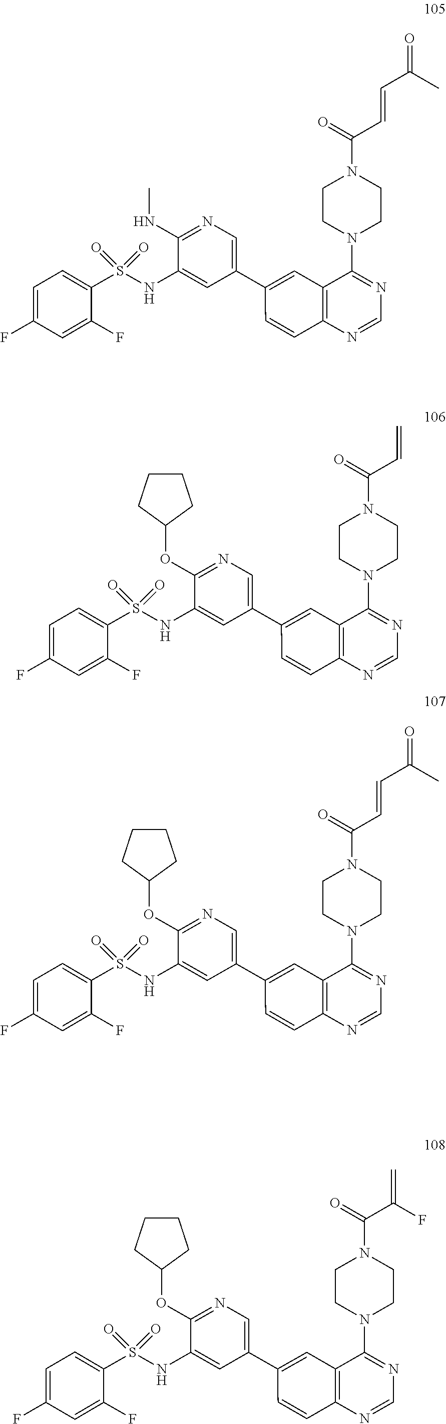 [SMILES: CNc1ncc(-c2ccc3ncnc(N4CCN(C(=O)/C=C/C(C)=O)CC4)c3c2)cc1NS(=O)(=O)c1ccc(F)cc1F ; C=CC(=O)N1CCN(c2ncnc3ccc(-c4cnc(OC5CCCC5)c(NS(=O)(=O)c5ccc(F)cc5F)c4)cc23)CC1 ; CC(=O)/C=C/C(=O)N1CCN(c2ncnc3ccc(-c4cnc(OC5CCCC5)c(NS(=O)(=O)c5ccc(F)cc5F)c4)cc23)CC1 ; C=C(F)C(=O)N1CCN(c2ncnc3ccc(-c4cnc(OC5CCCC5)c(NS(=O)(=O)c5ccc(F)cc5F)c4)cc23)CC1]

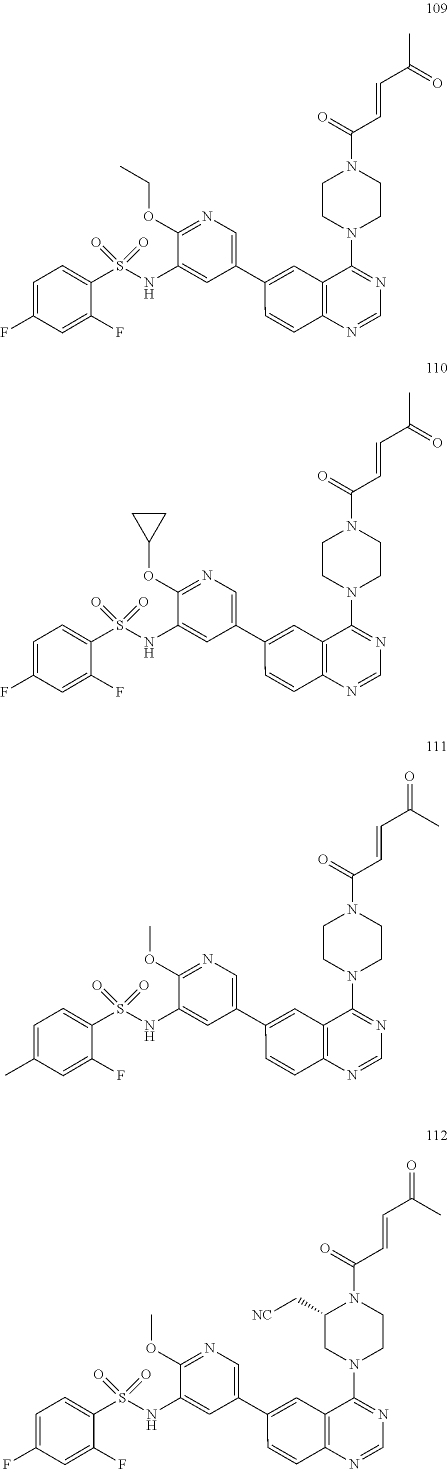 [SMILES: CCOc1ncc(-c2ccc3ncnc(N4CCN(C(=O)/C=C/C(C)=O)CC4)c3c2)cc1NS(=O)(=O)c1ccc(F)cc1F ; CC(=O)/C=C/C(=O)N1CCN(c2ncnc3ccc(-c4cnc(OC5CC5)c(NS(=O)(=O)c5ccc(F)cc5F)c4)cc23)CC1 ; COc1ncc(-c2ccc3ncnc(N4CCN(C(=O)/C=C/C(C)=O)CC4)c3c2)cc1NS(=O)(=O)c1ccc(C)cc1F ; COc1ncc(-c2ccc3ncnc(N4CCN(C(=O)/C=C/C(C)=O)[C@@H](CC#N)C4)c3c2)cc1NS(=O)(=O)c1ccc(F)cc1F]

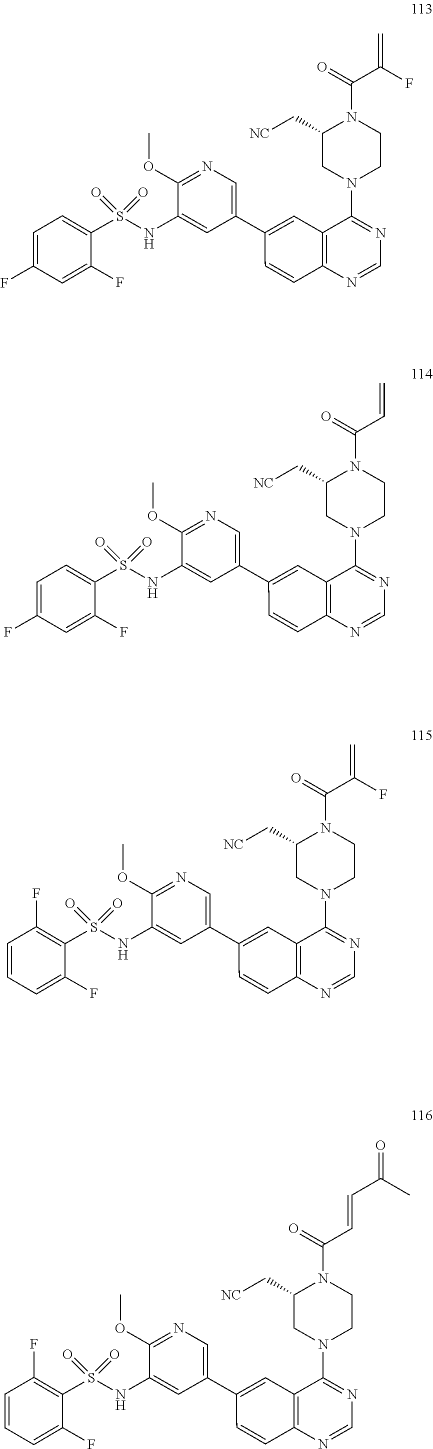 [SMILES: C=C(F)C(=O)N1CCN(c2ncnc3ccc(-c4cnc(OC)c(NS(=O)(=O)c5ccc(F)cc5F)c4)cc23)C[C@@H]1CC#N ; C=CC(=O)N1CCN(c2ncnc3ccc(-c4cnc(OC)c(NS(=O)(=O)c5ccc(F)cc5F)c4)cc23)C[C@@H]1CC#N ; C=C(F)C(=O)N1CCN(c2ncnc3ccc(-c4cnc(OC)c(NS(=O)(=O)c5c(F)cccc5F)c4)cc23)C[C@@H]1CC#N ; COc1ncc(-c2ccc3ncnc(N4CCN(C(=O)/C=C/C(C)=O)[C@@H](CC#N)C4)c3c2)cc1NS(=O)(=O)c1c(F)cccc1F]

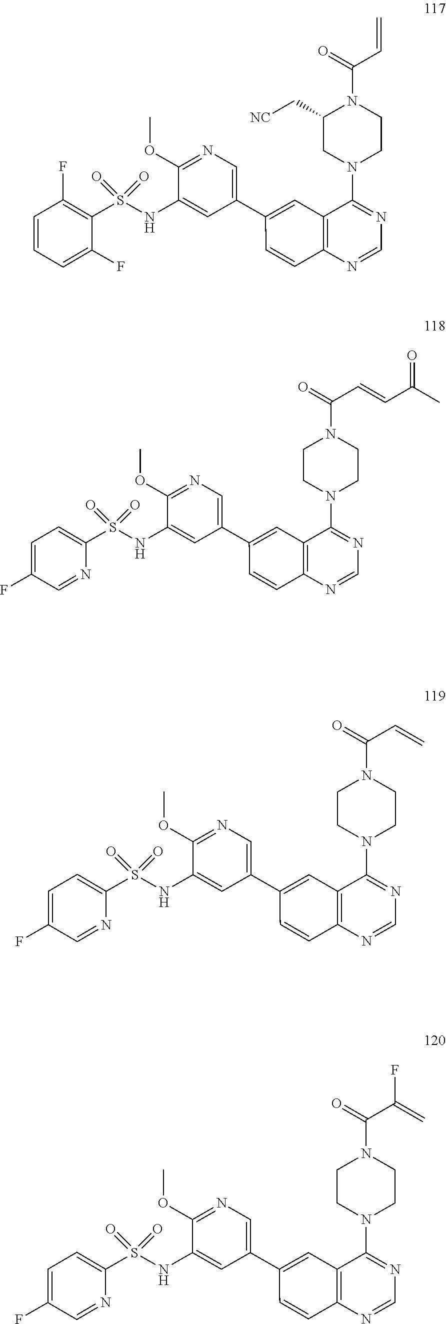 [SMILES: C=CC(=O)N1CCN(c2ncnc3ccc(-c4cnc(OC)c(NS(=O)(=O)c5c(F)cccc5F)c4)cc23)C[C@@H]1CC#N ; COc1ncc(-c2ccc3ncnc(N4CCN(C(=O)/C=C/C(C)=O)CC4)c3c2)cc1NS(=O)(=O)c1ccc(F)cn1 ; C=CC(=O)N1CCN(c2ncnc3ccc(-c4cnc(OC)c(NS(=O)(=O)c5ccc(F)cn5)c4)cc23)CC1 ; C=C(F)C(=O)N1CCN(c2ncnc3ccc(-c4cnc(OC)c(NS(=O)(=O)c5ccc(F)cn5)c4)cc23)CC1]

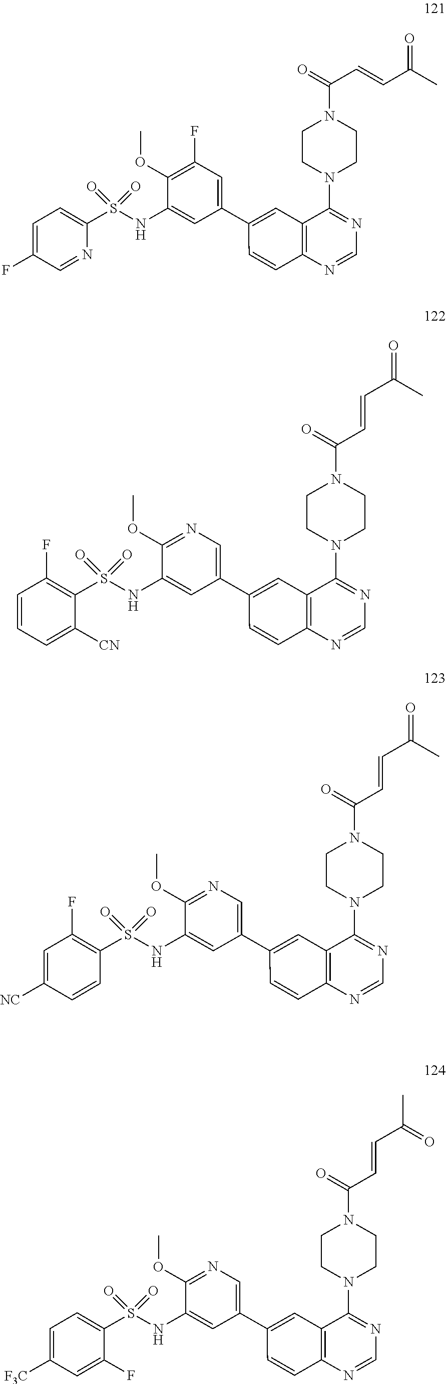 [SMILES: COc1c(F)cc(-c2ccc3ncnc(N4CCN(C(=O)/C=C/C(C)=O)CC4)c3c2)cc1NS(=O)(=O)c1ccc(F)cn1 ; COc1ncc(-c2ccc3ncnc(N4CCN(C(=O)/C=C/C(C)=O)CC4)c3c2)cc1NS(=O)(=O)c1c(F)cccc1C#N ; COc1ncc(-c2ccc3ncnc(N4CCN(C(=O)/C=C/C(C)=O)CC4)c3c2)cc1NS(=O)(=O)c1ccc(C#N)cc1F ; COc1ncc(-c2ccc3ncnc(N4CCN(C(=O)/C=C/C(C)=O)CC4)c3c2)cc1NS(=O)(=O)c1ccc(C(F)(F)F)cc1F]

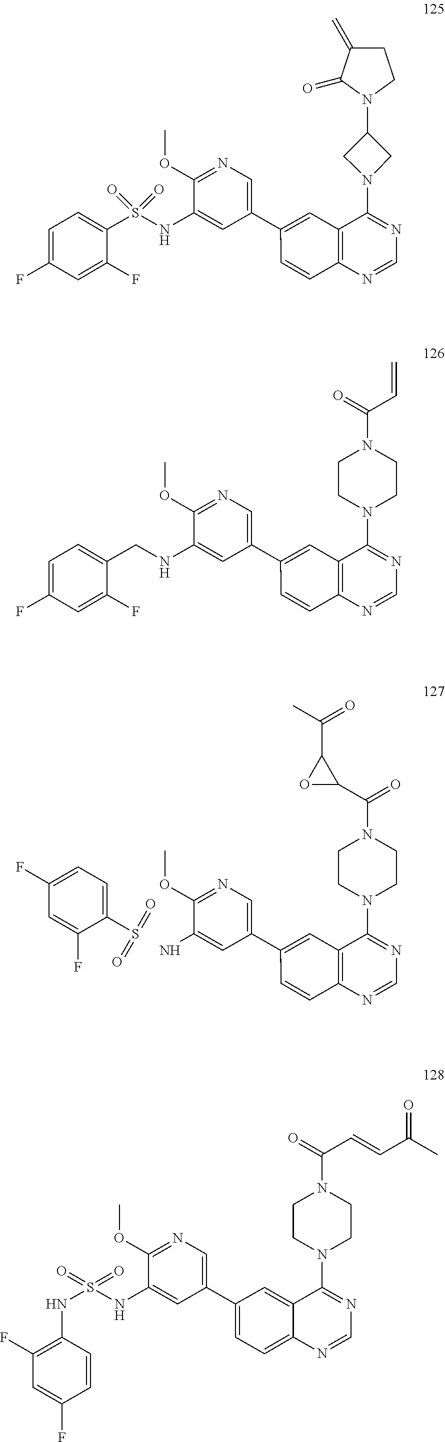

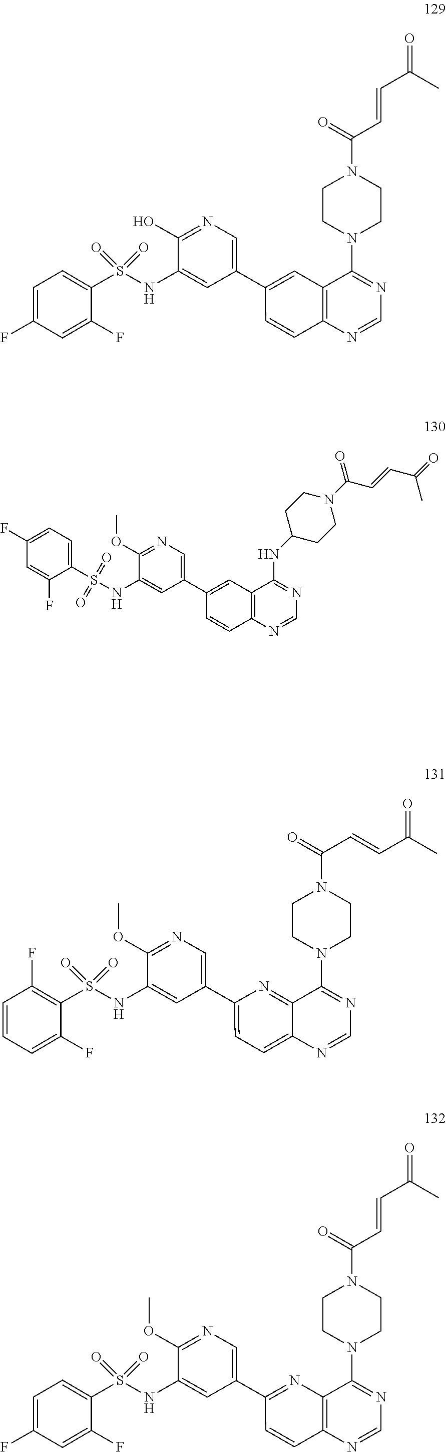 [SMILES: CC(=O)/C=C/C(=O)N1CCN(c2ncnc3ccc(-c4cnc(O)c(NS(=O)(=O)c5ccc(F)cc5F)c4)cc23)CC1 ; COc1ncc(-c2ccc3ncnc(NC4CCN(C(=O)/C=C/C(C)=O)CC4)c3c2)cc1NS(=O)(=O)c1ccc(F)cc1F ; COc1ncc(-c2ccc3ncnc(N4CCN(C(=O)/C=C/C(C)=O)CC4)c3n2)cc1NS(=O)(=O)c1c(F)cccc1F ; COc1ncc(-c2ccc3ncnc(N4CCN(C(=O)/C=C/C(C)=O)CC4)c3n2)cc1NS(=O)(=O)c1ccc(F)cc1F]

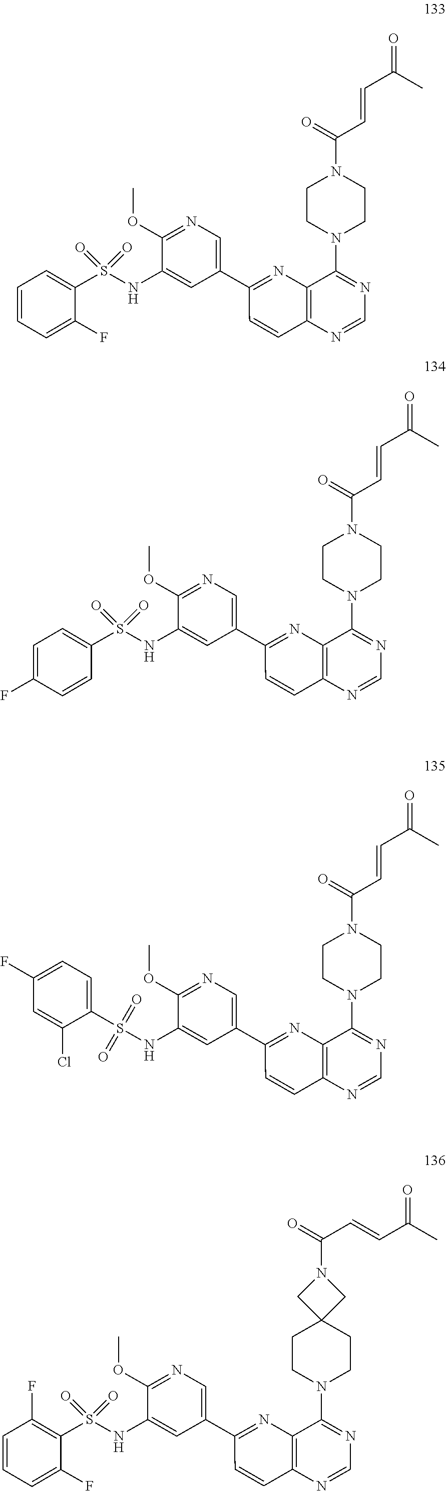 [SMILES: COc1ncc(-c2ccc3ncnc(N4CCN(C(=O)/C=C/C(C)=O)CC4)c3n2)cc1NS(=O)(=O)c1ccccc1F ; COc1ncc(-c2ccc3ncnc(N4CCN(C(=O)/C=C/C(C)=O)CC4)c3n2)cc1NS(=O)(=O)c1ccc(F)cc1 ; COc1ncc(-c2ccc3ncnc(N4CCN(C(=O)/C=C/C(C)=O)CC4)c3n2)cc1NS(=O)(=O)c1ccc(F)cc1Cl ; COc1ncc(-c2ccc3ncnc(N4CCC5(CC4)CN(C(=O)/C=C/C(C)=O)C5)c3n2)cc1NS(=O)(=O)c1c(F)cccc1F]

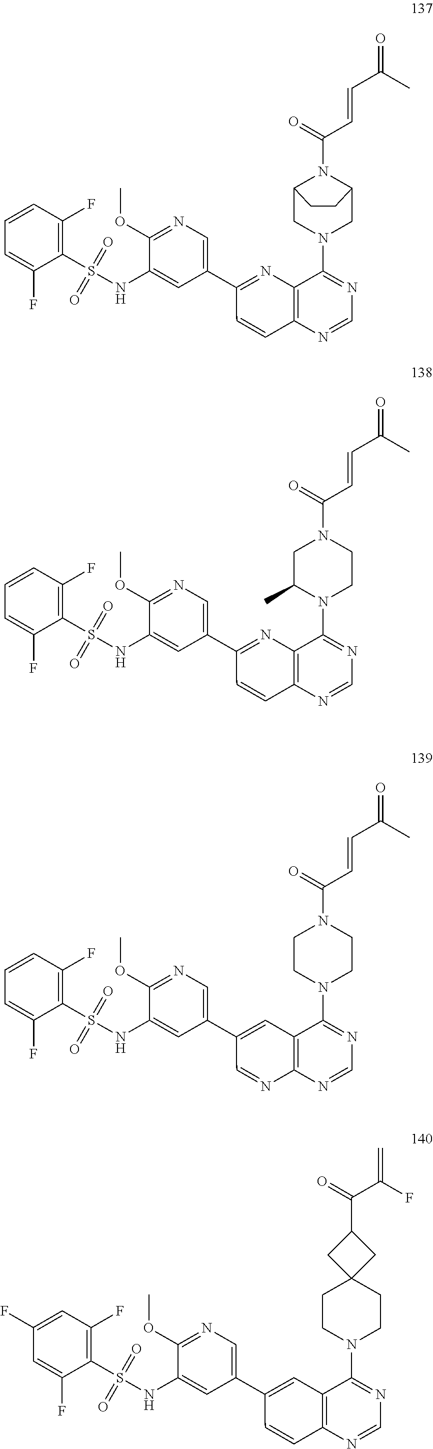 [SMILES: COc1ncc(-c2ccc3ncnc(N4CC5CCC(C4)N5C(=O)/C=C/C(C)=O)c3n2)cc1NS(=O)(=O)c1c(F)cccc1F ; COc1ncc(-c2ccc3ncnc(N4CCN(C(=O)/C=C/C(C)=O)C[C@@H]4C)c3n2)cc1NS(=O)(=O)c1c(F)cccc1F ; COc1ncc(-c2cnc3ncnc(N4CCN(C(=O)/C=C/C(C)=O)CC4)c3c2)cc1NS(=O)(=O)c1c(F)cccc1F ; C=C(F)C(=O)C1CC2(CCN(c3ncnc4ccc(-c5cnc(OC)c(NS(=O)(=O)c6c(F)cc(F)cc6F)c5)cc34)CC2)C1]

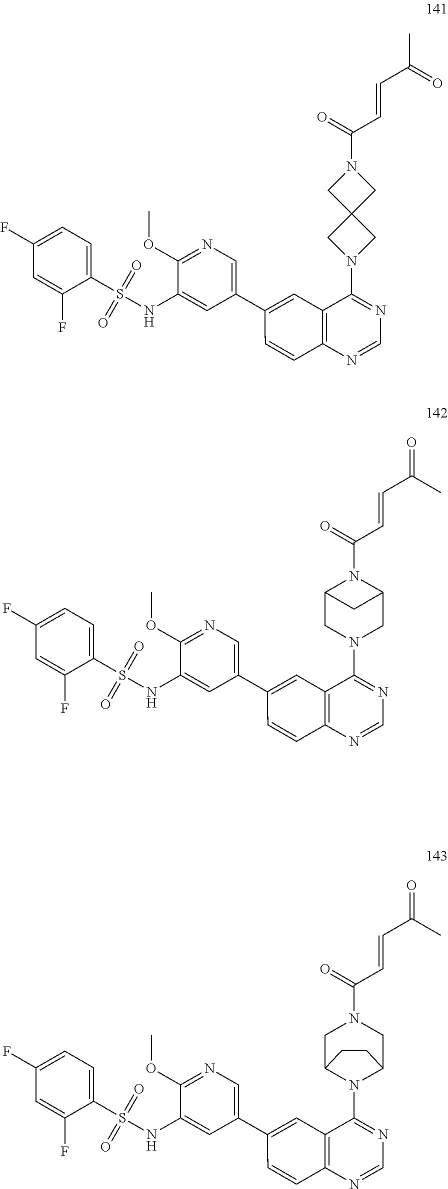 [SMILES: COc1ncc(-c2ccc3ncnc(N4CC5(CN(C(=O)/C=C/C(C)=O)C5)C4)c3c2)cc1NS(=O)(=O)c1ccc(F)cc1F ; COc1ncc(-c2ccc3ncnc(N4CC5CC(C4)N5C(=O)/C=C/C(C)=O)c3c2)cc1NS(=O)(=O)c1ccc(F)cc1F ; COc1ncc(-c2ccc3ncnc(N4C5CCC4CN(C(=O)/C=C/C(C)=O)C5)c3c2)cc1NS(=O)(=O)c1ccc(F)cc1F]

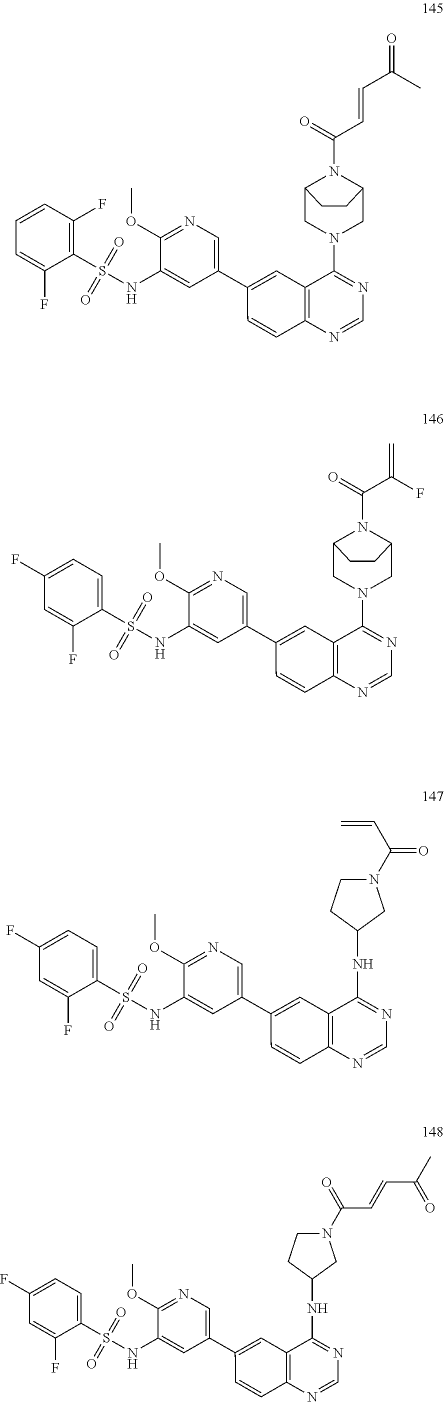 [SMILES: COc1ncc(-c2ccc3ncnc(N4CC5CCC(C4)N5C(=O)/C=C/C(C)=O)c3c2)cc1NS(=O)(=O)c1c(F)cccc1F ; C=C(F)C(=O)N1C2CCC1CN(c1ncnc3ccc(-c4cnc(OC)c(NS(=O)(=O)c5ccc(F)cc5F)c4)cc13)C2 ; C=CC(=O)N1CCC(Nc2ncnc3ccc(-c4cnc(OC)c(NS(=O)(=O)c5ccc(F)cc5F)c4)cc23)C1 ; COc1ncc(-c2ccc3ncnc(NC4CCN(C(=O)/C=C/C(C)=O)C4)c3c2)cc1NS(=O)(=O)c1ccc(F)cc1F]

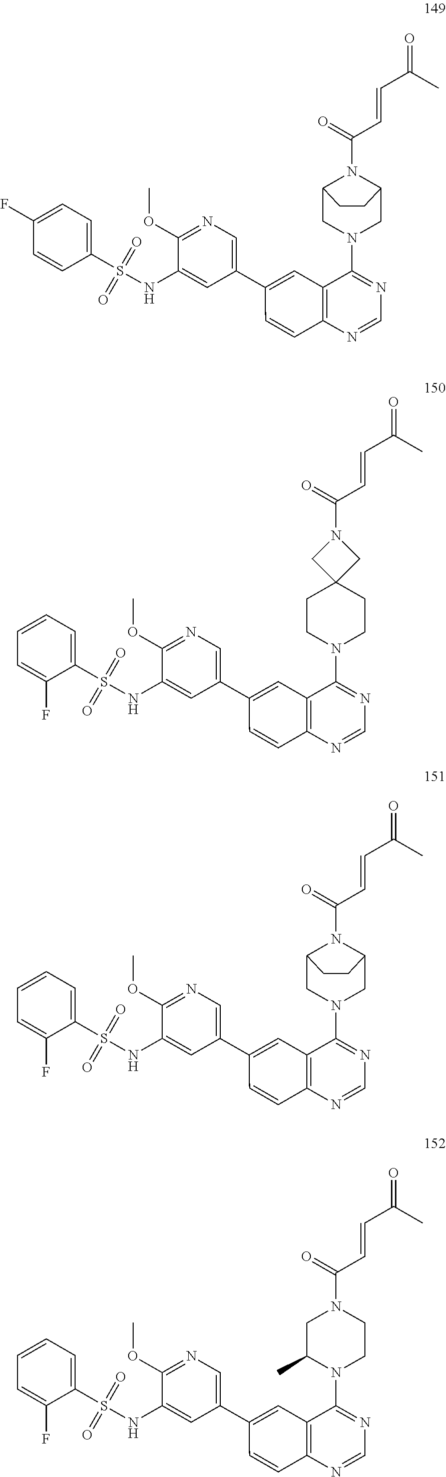 [SMILES: COc1ncc(-c2ccc3ncnc(N4CC5CCC(C4)N5C(=O)/C=C/C(C)=O)c3c2)cc1NS(=O)(=O)c1ccc(F)cc1 ; COc1ncc(-c2ccc3ncnc(N4CCC5(CC4)CN(C(=O)/C=C/C(C)=O)C5)c3c2)cc1NS(=O)(=O)c1ccccc1F ; COc1ncc(-c2ccc3ncnc(N4CC5CCC(C4)N5C(=O)/C=C/C(C)=O)c3c2)cc1NS(=O)(=O)c1ccccc1F ; COc1ncc(-c2ccc3ncnc(N4CCN(C(=O)/C=C/C(C)=O)C[C@@H]4C)c3c2)cc1NS(=O)(=O)c1ccccc1F]

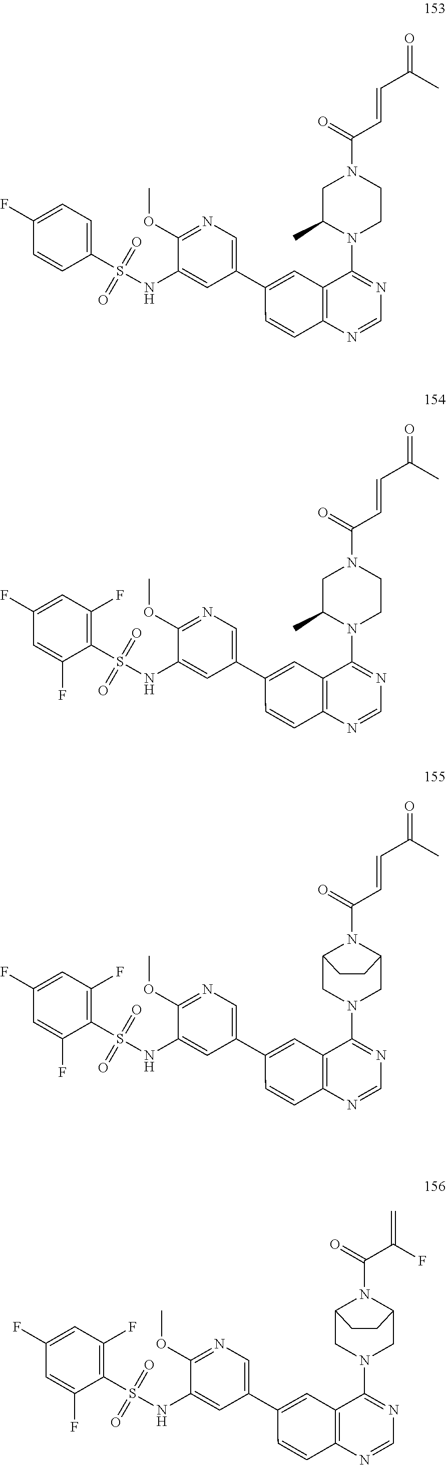 [SMILES: COc1ncc(-c2ccc3ncnc(N4CCN(C(=O)/C=C/C(C)=O)C[C@@H]4C)c3c2)cc1NS(=O)(=O)c1ccc(F)cc1 ; COc1ncc(-c2ccc3ncnc(N4CCN(C(=O)/C=C/C(C)=O)C[C@@H]4C)c3c2)cc1NS(=O)(=O)c1c(F)cc(F)cc1F ; COc1ncc(-c2ccc3ncnc(N4CC5CCC(C4)N5C(=O)/C=C/C(C)=O)c3c2)cc1NS(=O)(=O)c1c(F)cc(F)cc1F ; C=C(F)C(=O)N1C2CCC1CN(c1ncnc3ccc(-c4cnc(OC)c(NS(=O)(=O)c5c(F)cc(F)cc5F)c4)cc13)C2]

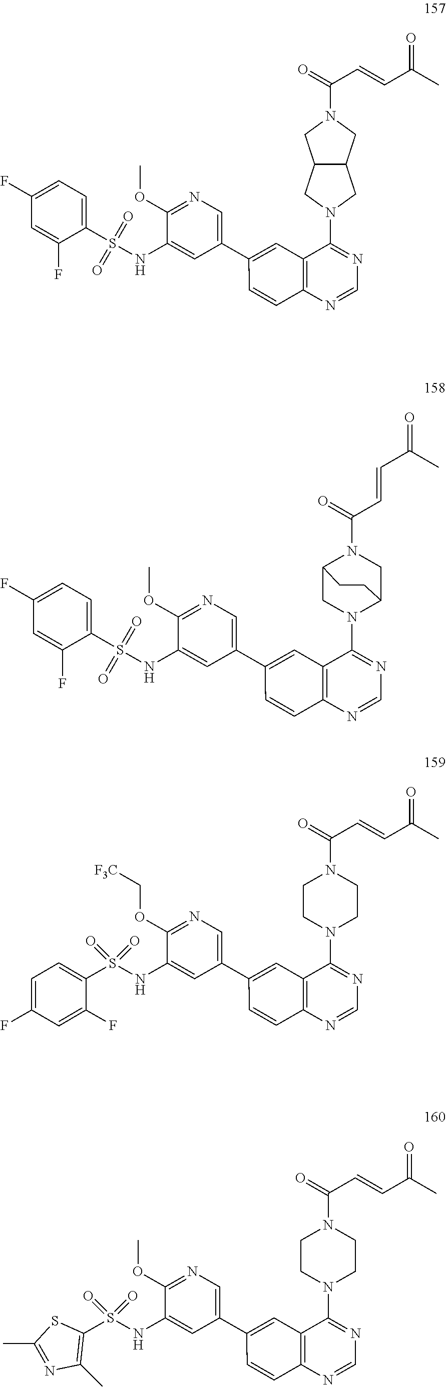

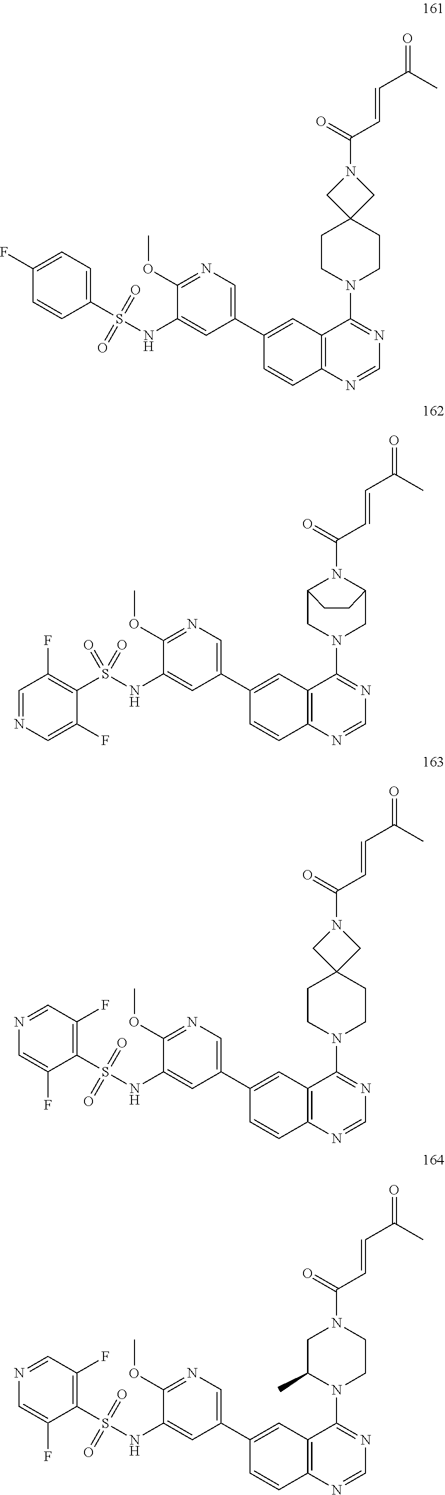 [SMILES: COc1ncc(-c2ccc3ncnc(N4CCC5(CC4)CN(C(=O)/C=C/C(C)=O)C5)c3c2)cc1NS(=O)(=O)c1ccc(F)cc1 ; COc1ncc(-c2ccc3ncnc(N4CC5CCC(C4)N5C(=O)/C=C/C(C)=O)c3c2)cc1NS(=O)(=O)c1c(F)cncc1F ; COc1ncc(-c2ccc3ncnc(N4CCC5(CC4)CN(C(=O)/C=C/C(C)=O)C5)c3c2)cc1NS(=O)(=O)c1c(F)cncc1F ; COc1ncc(-c2ccc3ncnc(N4CCN(C(=O)/C=C/C(C)=O)C[C@@H]4C)c3c2)cc1NS(=O)(=O)c1c(F)cncc1F]

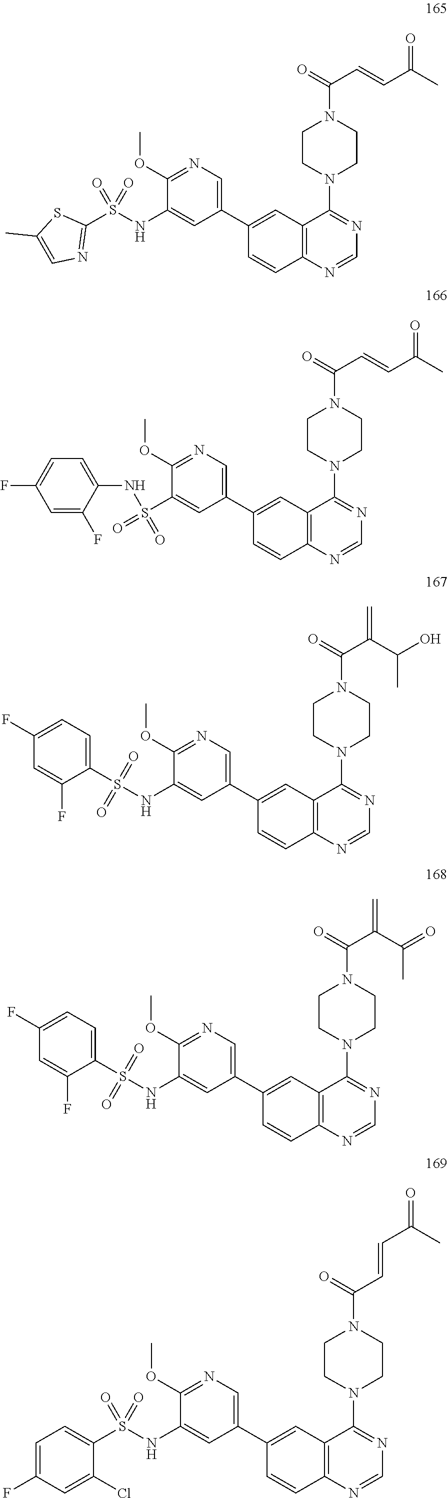 [SMILES: COc1ncc(-c2ccc3ncnc(N4CCN(C(=O)/C=C/C(C)=O)CC4)c3c2)cc1NS(=O)(=O)c1ncc(C)s1 ; COc1ncc(-c2ccc3ncnc(N4CCN(C(=O)/C=C/C(C)=O)CC4)c3c2)cc1S(=O)(=O)Nc1ccc(F)cc1F ; C=C(C(=O)N1CCN(c2ncnc3ccc(-c4cnc(OC)c(NS(=O)(=O)c5ccc(F)cc5F)c4)cc23)CC1)C(C)O ; C=C(C(C)=O)C(=O)N1CCN(c2ncnc3ccc(-c4cnc(OC)c(NS(=O)(=O)c5ccc(F)cc5F)c4)cc23)CC1 ; COc1ncc(-c2ccc3ncnc(N4CCN(C(=O)/C=C/C(C)=O)CC4)c3c2)cc1NS(=O)(=O)c1ccc(F)cc1Cl]

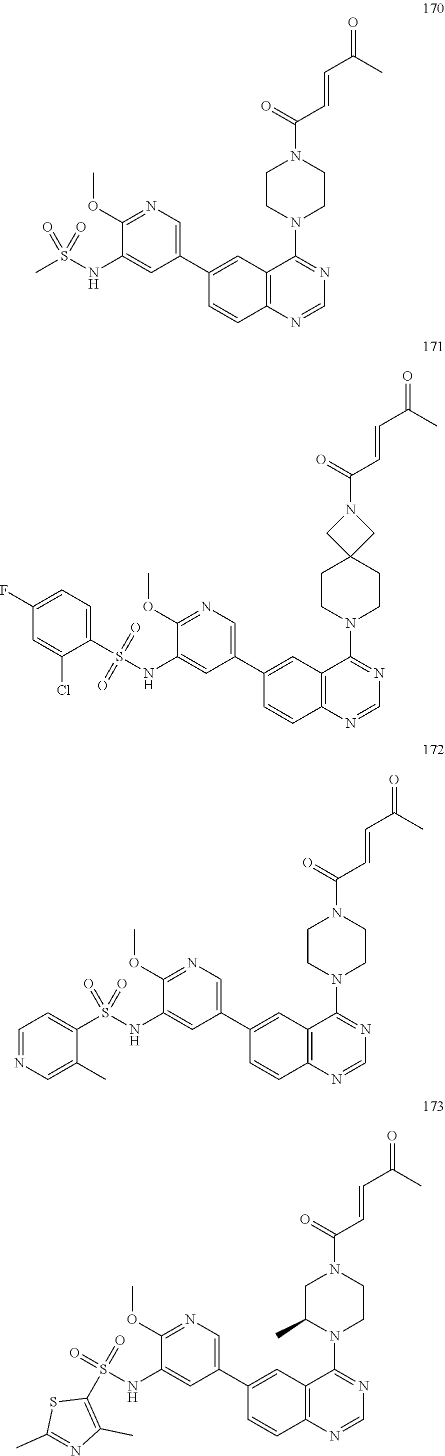

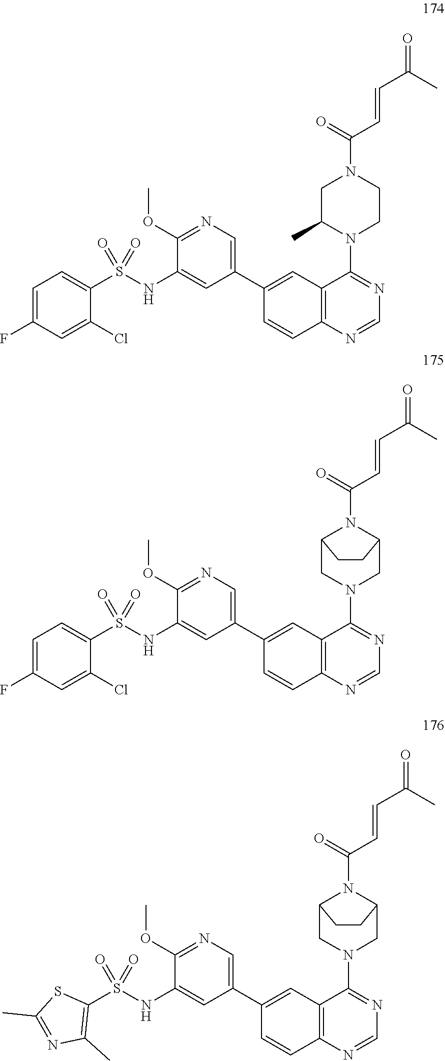

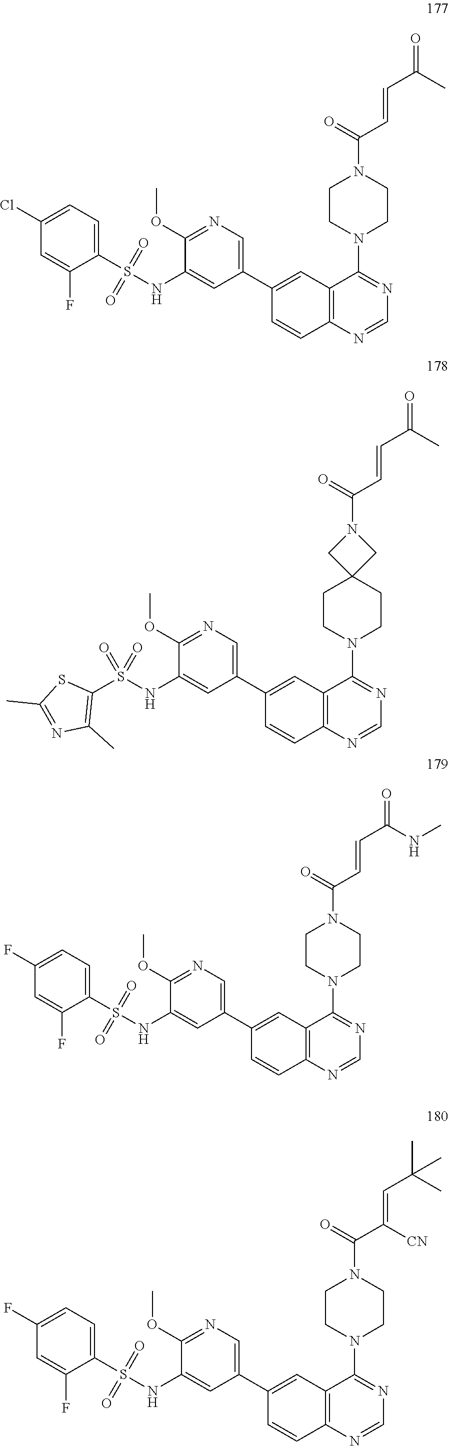

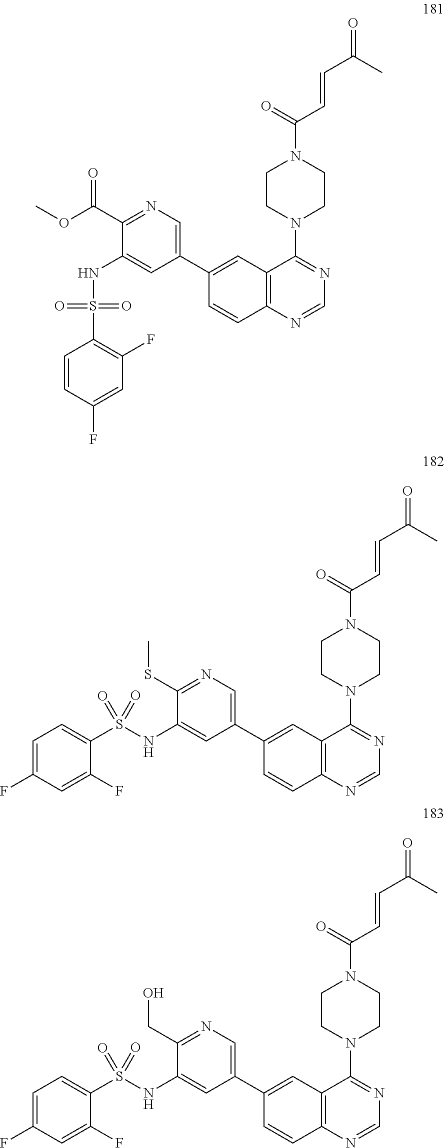 [SMILES: COC(=O)c1ncc(-c2ccc3ncnc(N4CCN(C(=O)/C=C/C(C)=O)CC4)c3c2)cc1NS(=O)(=O)c1ccc(F)cc1F ; CSc1ncc(-c2ccc3ncnc(N4CCN(C(=O)/C=C/C(C)=O)CC4)c3c2)cc1NS(=O)(=O)c1ccc(F)cc1F ; CC(=O)/C=C/C(=O)N1CCN(c2ncnc3ccc(-c4cnc(CO)c(NS(=O)(=O)c5ccc(F)cc5F)c4)cc23)CC1]

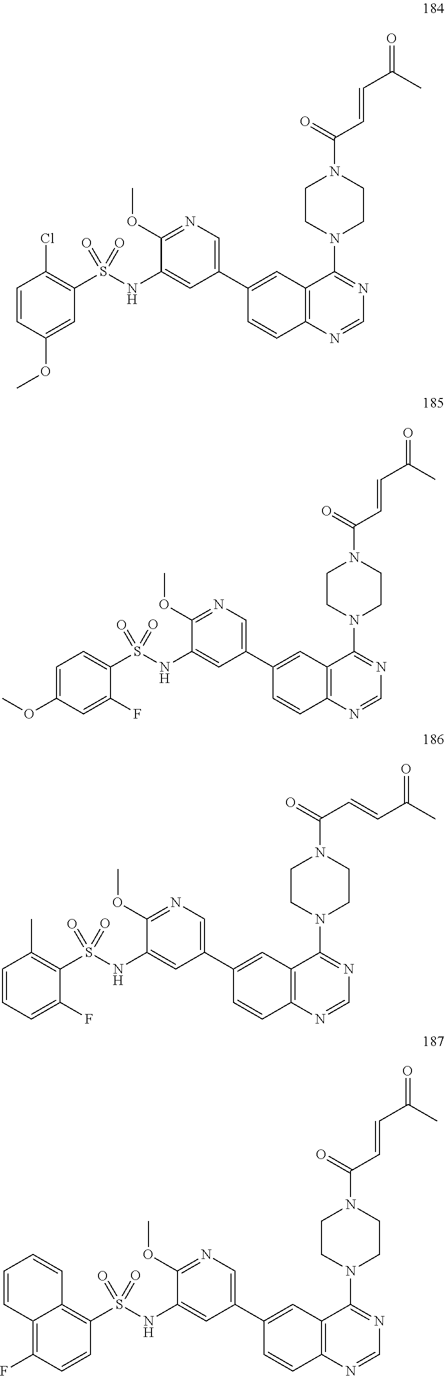

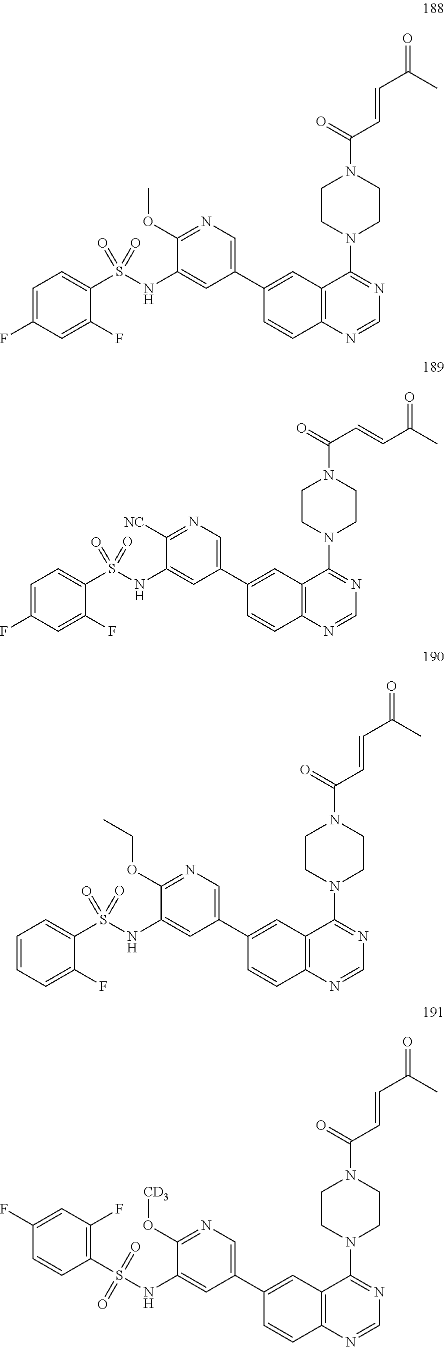 [SMILES: COc1ncc(-c2ccc3ncnc(N4CCN(C(=O)/C=C/C(C)=O)CC4)c3c2)cc1NS(=O)(=O)c1ccc(F)cc1F ; CC(=O)/C=C/C(=O)N1CCN(c2ncnc3ccc(-c4cnc(C#N)c(NS(=O)(=O)c5ccc(F)cc5F)c4)cc23)CC1 ; CCOc1ncc(-c2ccc3ncnc(N4CCN(C(=O)/C=C/C(C)=O)CC4)c3c2)cc1NS(=O)(=O)c1ccccc1F ; [2H]C([2H])([2H])Oc1ncc(-c2ccc3ncnc(N4CCN(C(=O)/C=C/C(C)=O)CC4)c3c2)cc1NS(=O)(=O)c1ccc(F)cc1F]

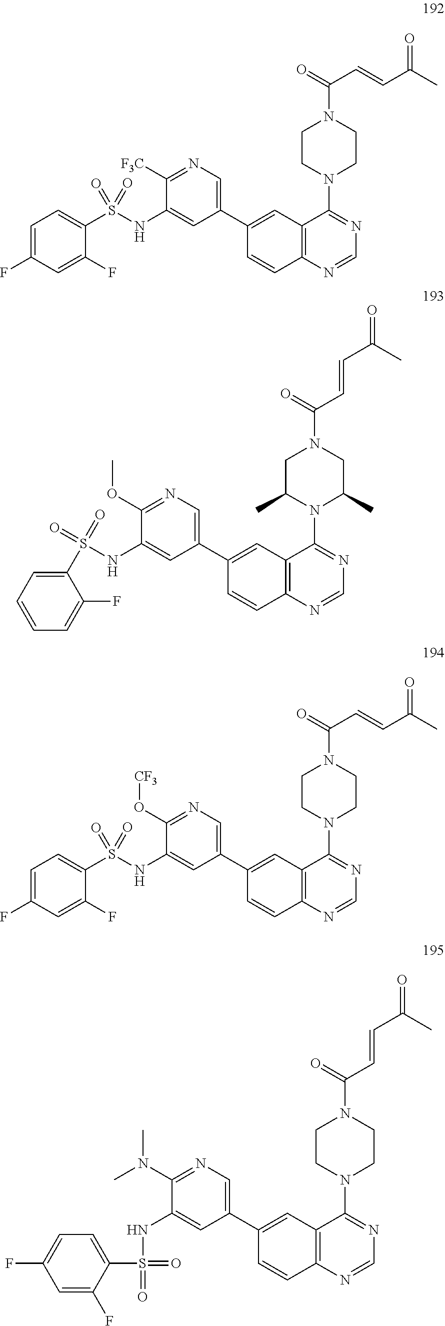 [SMILES: CC(=O)/C=C/C(=O)N1CCN(c2ncnc3ccc(-c4cnc(C(F)(F)F)c(NS(=O)(=O)c5ccc(F)cc5F)c4)cc23)CC1 ; COc1ncc(-c2ccc3ncnc(N4[C@H](C)CN(C(=O)/C=C/C(C)=O)C[C@@H]4C)c3c2)cc1NS(=O)(=O)c1ccccc1F ; CC(=O)/C=C/C(=O)N1CCN(c2ncnc3ccc(-c4cnc(OC(F)(F)F)c(NS(=O)(=O)c5ccc(F)cc5F)c4)cc23)CC1 ; CC(=O)/C=C/C(=O)N1CCN(c2ncnc3ccc(-c4cnc(N(C)C)c(NS(=O)(=O)c5ccc(F)cc5F)c4)cc23)CC1]

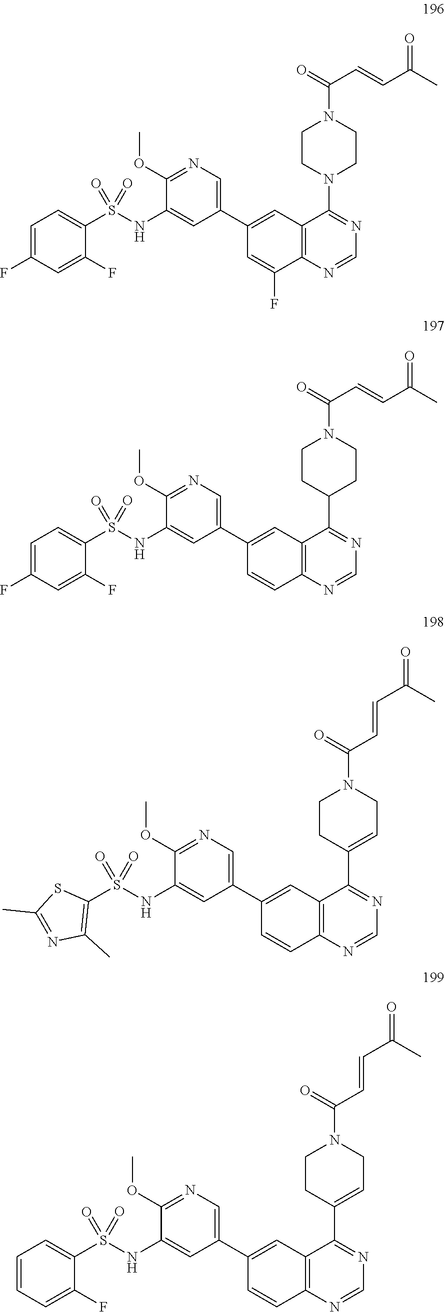

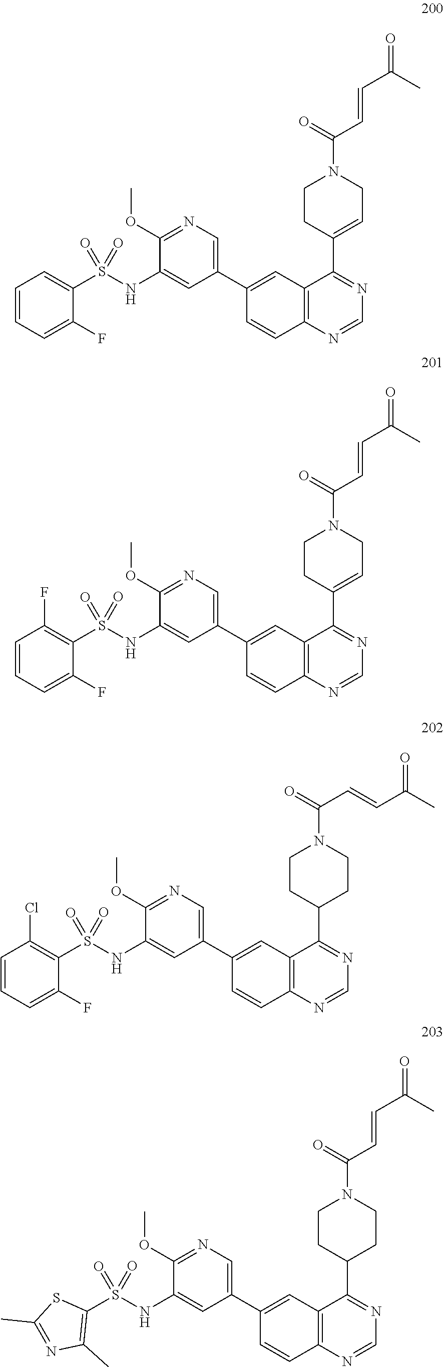

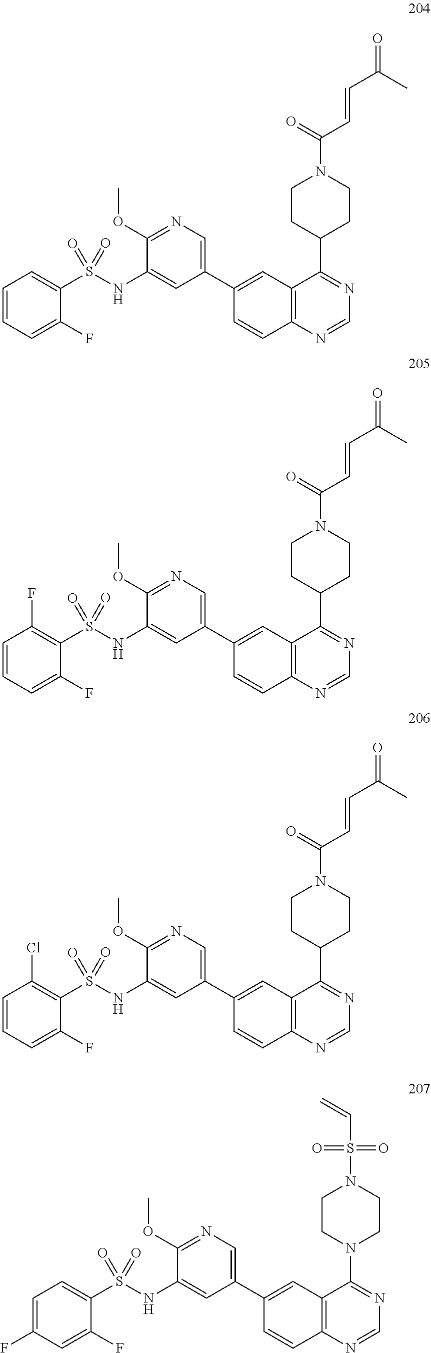 [SMILES: COc1ncc(-c2ccc3ncnc(C4CCN(C(=O)/C=C/C(C)=O)CC4)c3c2)cc1NS(=O)(=O)c1ccccc1F ; COc1ncc(-c2ccc3ncnc(C4CCN(C(=O)/C=C/C(C)=O)CC4)c3c2)cc1NS(=O)(=O)c1c(F)cccc1F ; COc1ncc(-c2ccc3ncnc(C4CCN(C(=O)/C=C/C(C)=O)CC4)c3c2)cc1NS(=O)(=O)c1c(F)cccc1Cl ; C=CS(=O)(=O)N1CCN(c2ncnc3ccc(-c4cnc(OC)c(NS(=O)(=O)c5ccc(F)cc5F)c4)cc23)CC1]

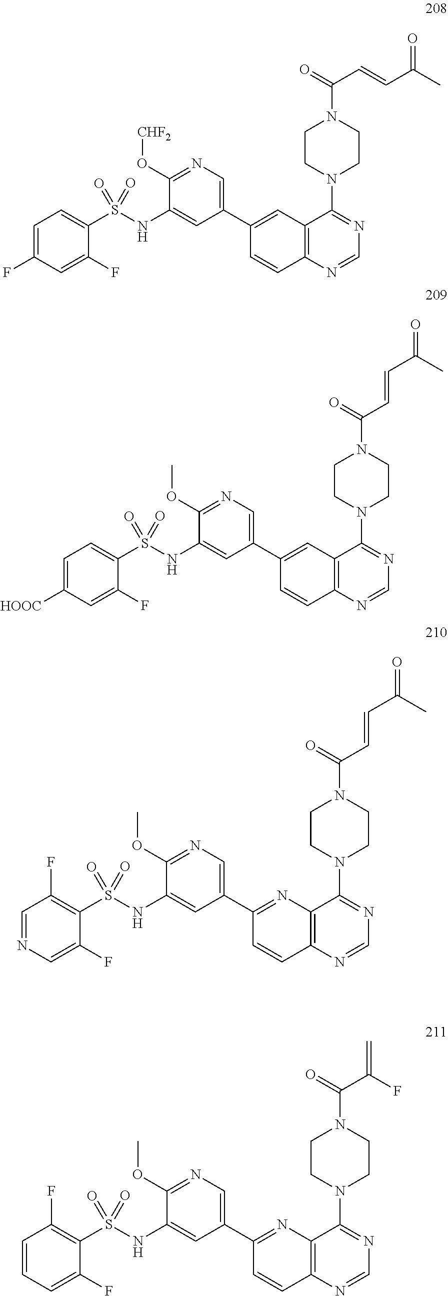 [SMILES: CC(=O)/C=C/C(=O)N1CCN(c2ncnc3ccc(-c4cnc(OC(F)F)c(NS(=O)(=O)c5ccc(F)cc5F)c4)cc23)CC1 ; COc1ncc(-c2ccc3ncnc(N4CCN(C(=O)/C=C/C(C)=O)CC4)c3c2)cc1NS(=O)(=O)c1ccc(C(=O)O)cc1F ; COc1ncc(-c2ccc3ncnc(N4CCN(C(=O)/C=C/C(C)=O)CC4)c3n2)cc1NS(=O)(=O)c1c(F)cncc1F ; C=C(F)C(=O)N1CCN(c2ncnc3ccc(-c4cnc(OC)c(NS(=O)(=O)c5c(F)cccc5F)c4)nc23)CC1]

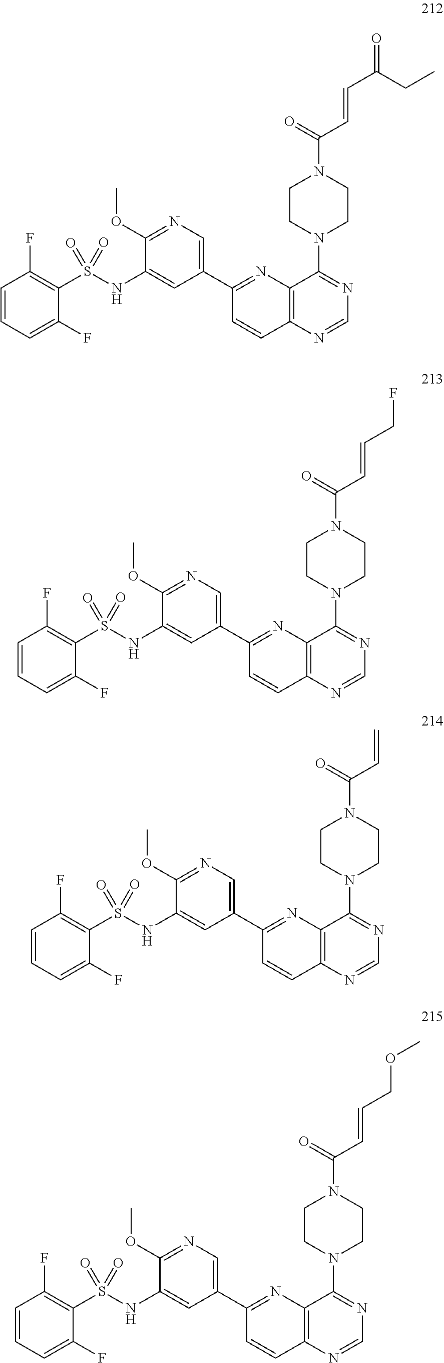 [SMILES: CCC(=O)/C=C/C(=O)N1CCN(c2ncnc3ccc(-c4cnc(OC)c(NS(=O)(=O)c5c(F)cccc5F)c4)nc23)CC1 ; COc1ncc(-c2ccc3ncnc(N4CCN(C(=O)/C=C/CF)CC4)c3n2)cc1NS(=O)(=O)c1c(F)cccc1F ; C=CC(=O)N1CCN(c2ncnc3ccc(-c4cnc(OC)c(NS(=O)(=O)c5c(F)cccc5F)c4)nc23)CC1 ; COC/C=C/C(=O)N1CCN(c2ncnc3ccc(-c4cnc(OC)c(NS(=O)(=O)c5c(F)cccc5F)c4)nc23)CC1]

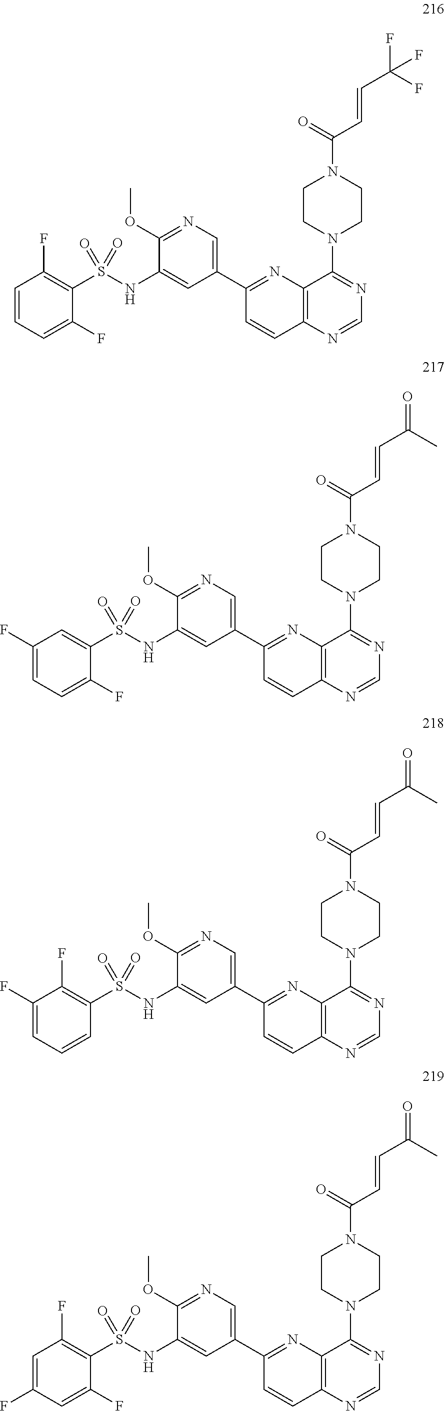 [SMILES: COc1ncc(-c2ccc3ncnc(N4CCN(C(=O)/C=C/C(F)(F)F)CC4)c3n2)cc1NS(=O)(=O)c1c(F)cccc1F ; COc1ncc(-c2ccc3ncnc(N4CCN(C(=O)/C=C/C(C)=O)CC4)c3n2)cc1NS(=O)(=O)c1cc(F)ccc1F ; COc1ncc(-c2ccc3ncnc(N4CCN(C(=O)/C=C/C(C)=O)CC4)c3n2)cc1NS(=O)(=O)c1cccc(F)c1F ; COc1ncc(-c2ccc3ncnc(N4CCN(C(=O)/C=C/C(C)=O)CC4)c3n2)cc1NS(=O)(=O)c1c(F)cc(F)cc1F]

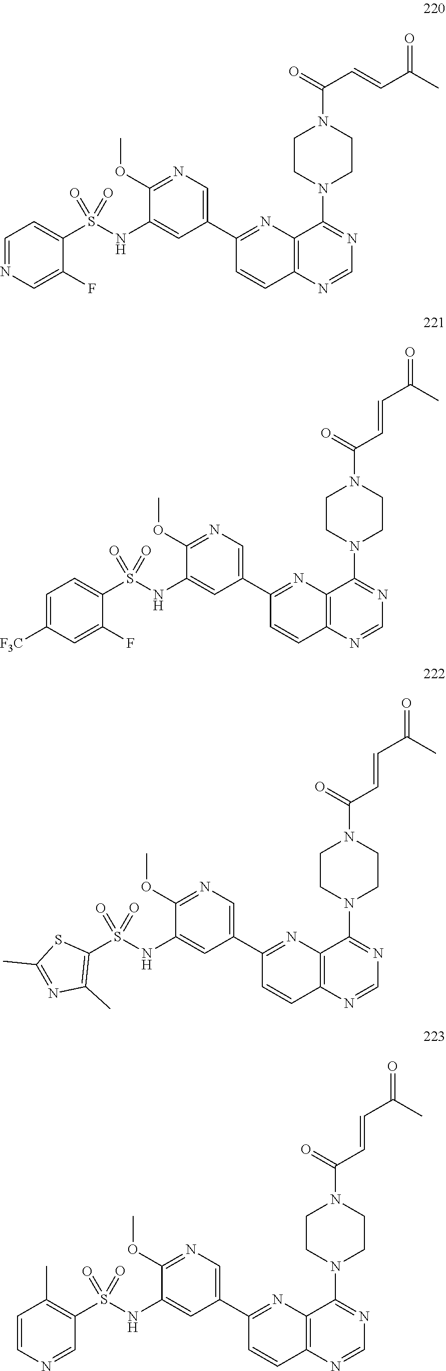

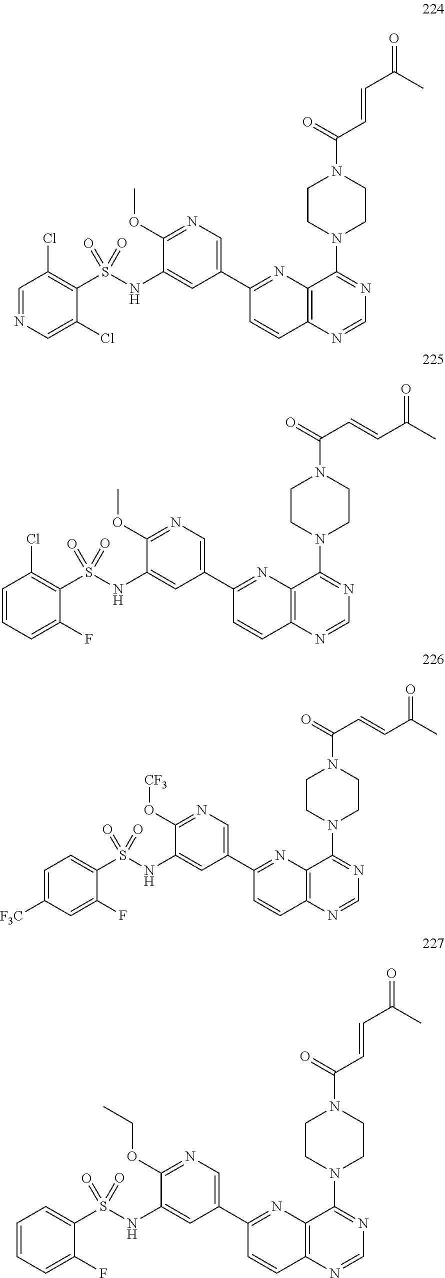 [SMILES: COc1ncc(-c2ccc3ncnc(N4CCN(C(=O)/C=C/C(C)=O)CC4)c3n2)cc1NS(=O)(=O)c1c(Cl)cncc1Cl ; COc1ncc(-c2ccc3ncnc(N4CCN(C(=O)/C=C/C(C)=O)CC4)c3n2)cc1NS(=O)(=O)c1c(F)cccc1Cl ; CC(=O)/C=C/C(=O)N1CCN(c2ncnc3ccc(-c4cnc(OC(F)(F)F)c(NS(=O)(=O)c5ccc(C(F)(F)F)cc5F)c4)nc23)CC1 ; CCOc1ncc(-c2ccc3ncnc(N4CCN(C(=O)/C=C/C(C)=O)CC4)c3n2)cc1NS(=O)(=O)c1ccccc1F]

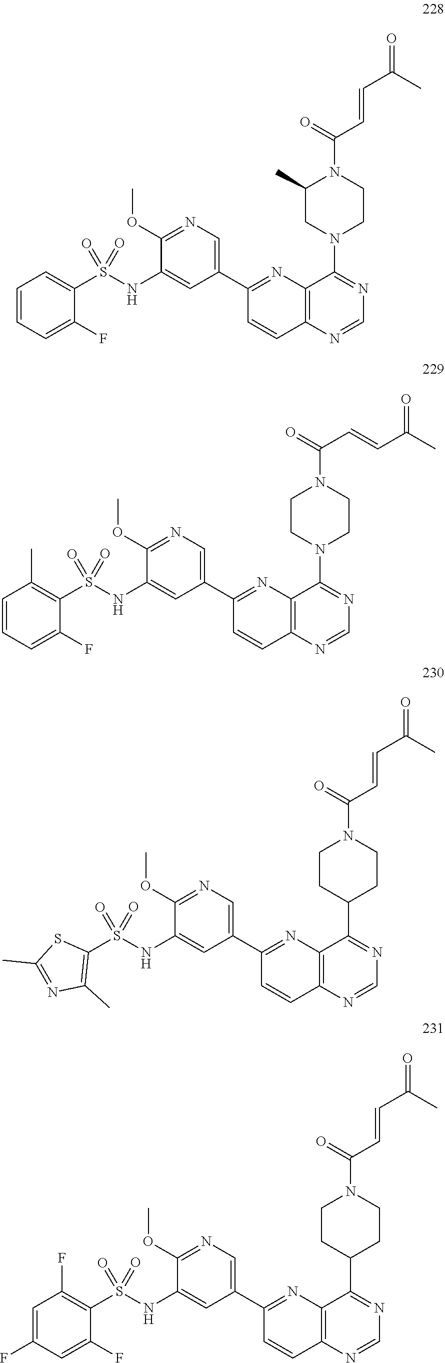

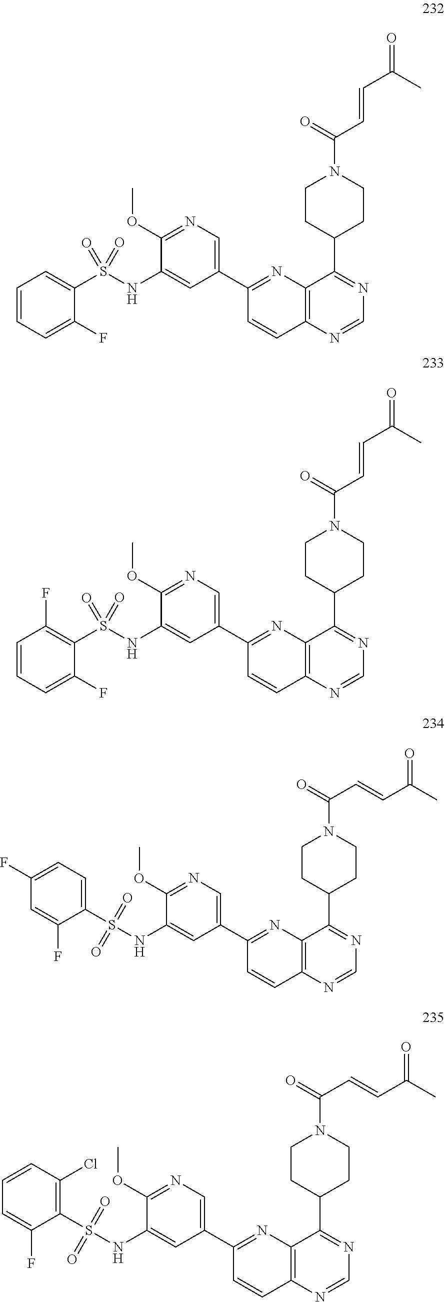 [SMILES: COc1ncc(-c2ccc3ncnc(C4CCN(C(=O)/C=C/C(C)=O)CC4)c3n2)cc1NS(=O)(=O)c1ccccc1F ; COc1ncc(-c2ccc3ncnc(C4CCN(C(=O)/C=C/C(C)=O)CC4)c3n2)cc1NS(=O)(=O)c1c(F)cccc1F ; COc1ncc(-c2ccc3ncnc(C4CCN(C(=O)/C=C/C(C)=O)CC4)c3n2)cc1NS(=O)(=O)c1ccc(F)cc1F ; COc1ncc(-c2ccc3ncnc(C4CCN(C(=O)/C=C/C(C)=O)CC4)c3n2)cc1NS(=O)(=O)c1c(F)cccc1Cl]

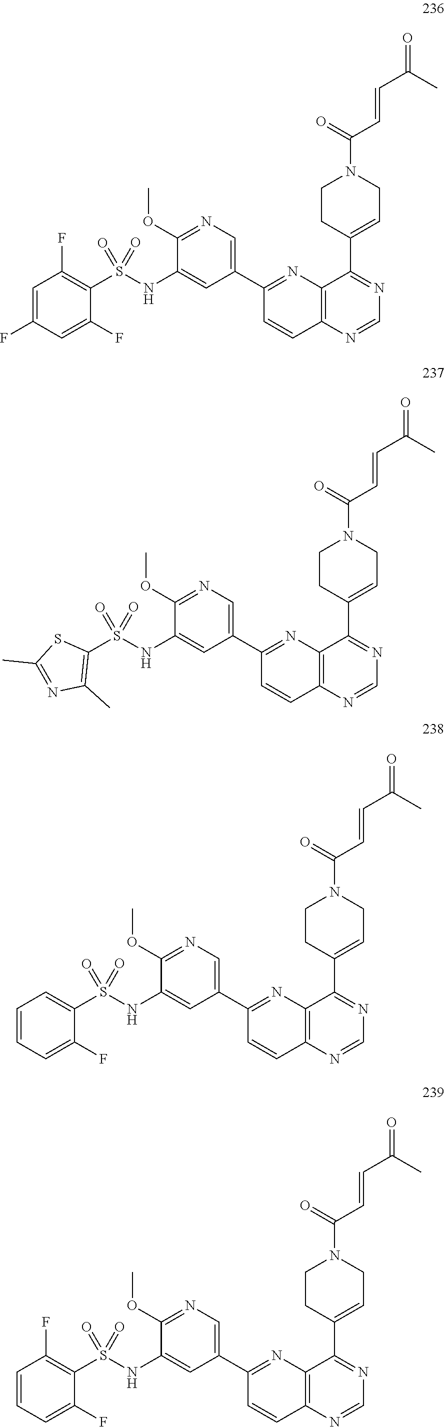

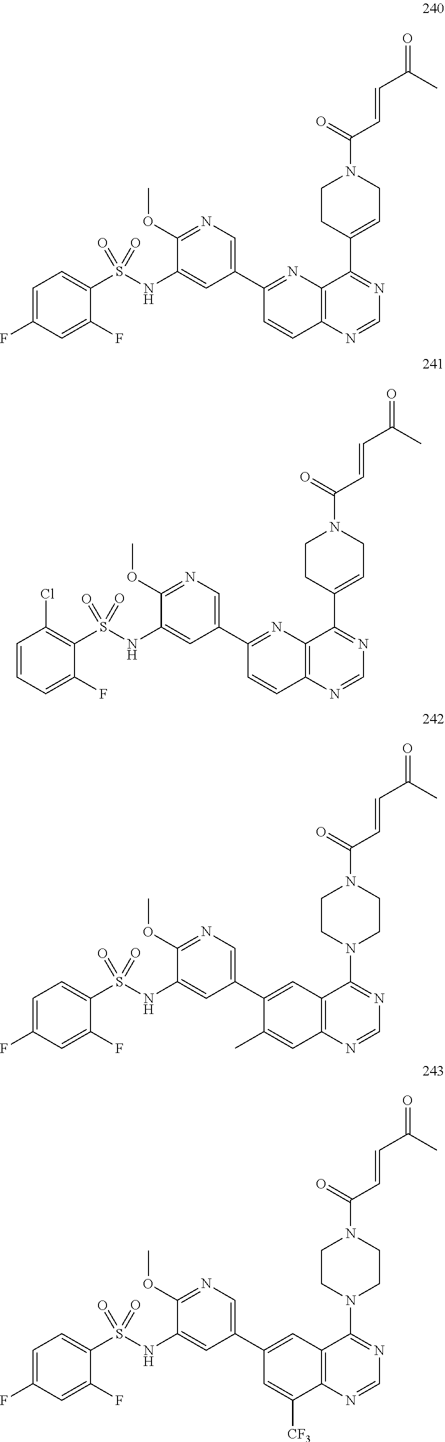 [SMILES: COc1ncc(-c2ccc3ncnc(C4=CCN(C(=O)/C=C/C(C)=O)CC4)c3n2)cc1NS(=O)(=O)c1ccc(F)cc1F ; COc1ncc(-c2ccc3ncnc(C4=CCN(C(=O)/C=C/C(C)=O)CC4)c3n2)cc1NS(=O)(=O)c1c(F)cccc1Cl ; COc1ncc(-c2cc3c(N4CCN(C(=O)/C=C/C(C)=O)CC4)ncnc3cc2C)cc1NS(=O)(=O)c1ccc(F)cc1F ; COc1ncc(-c2cc(C(F)(F)F)c3ncnc(N4CCN(C(=O)/C=C/C(C)=O)CC4)c3c2)cc1NS(=O)(=O)c1ccc(F)cc1F]

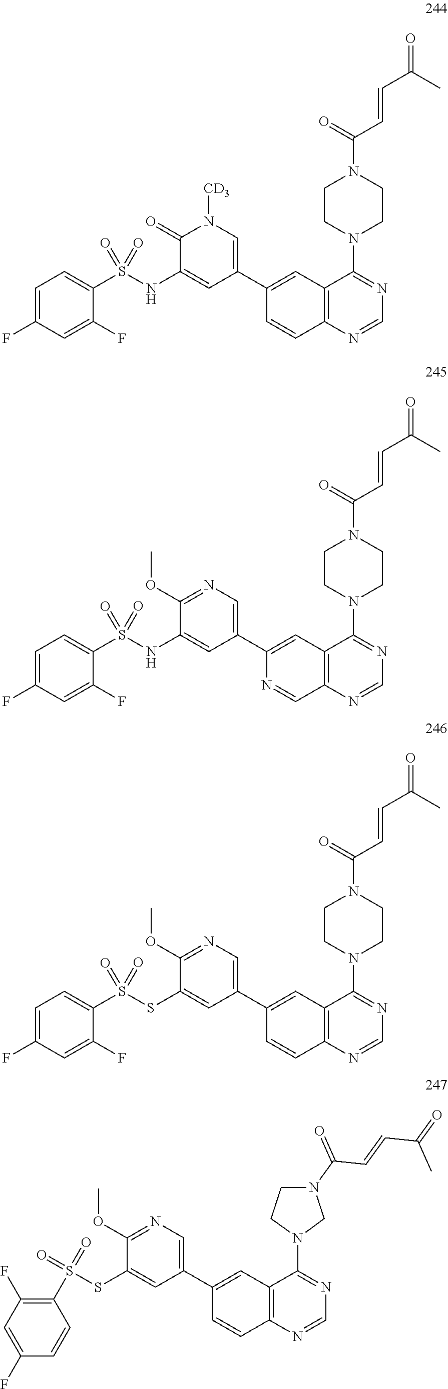 [SMILES: [2H]C([2H])([2H])n1cc(-c2ccc3ncnc(N4CCN(C(=O)/C=C/C(C)=O)CC4)c3c2)cc(NS(=O)(=O)c2ccc(F)cc2F)c1=O ; COc1ncc(-c2cc3c(N4CCN(C(=O)/C=C/C(C)=O)CC4)ncnc3cn2)cc1NS(=O)(=O)c1ccc(F)cc1F ; COc1ncc(-c2ccc3ncnc(N4CCN(C(=O)/C=C/C(C)=O)CC4)c3c2)cc1SS(=O)(=O)c1ccc(F)cc1F ; COc1ncc(-c2ccc3ncnc(N4CCN(C(=O)/C=C/C(C)=O)C4)c3c2)cc1SS(=O)(=O)c1ccc(F)cc1F]

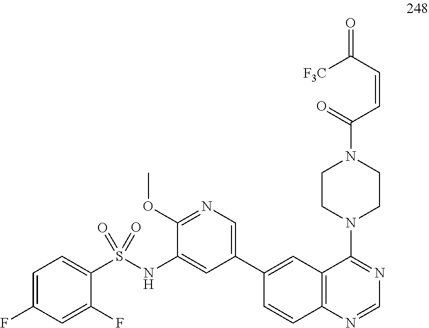 [SMILES: COc1ncc(-c2ccc3ncnc(N4CCN(C(=O)/C=C\C(=O)C(F)(F)F)CC4)c3c2)cc1NS(=O)(=O)c1ccc(F)cc1F]

The present disclosure also provides a preparation method of the above compound, which includes the following steps:

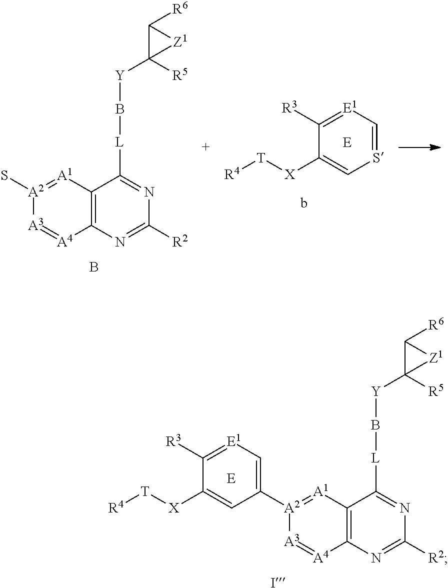

Wherein the definitions of each group in formulas (a) and (b) am as described above.

S and S′ are selected from halogen, boronic acid or boronic ester: provided that, when S is selected from halogen, S′ is selected from boronic acid or boronic ester: when S′ is selected from halogen, S is selected from boronic acid or boronic ester.

The compound of formula I″″ are prepared by Suzuki coupling reaction with the compound of formula (a) and formula (b).

The compounds of formula (a) and formula (b) can be prepared with reference to the examples. On the basis of the compound core

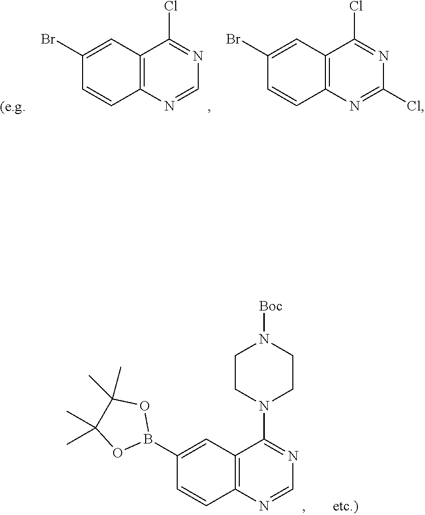

provided in the examples, according to the methods described in the examples and/or by using the intermediate raw materials

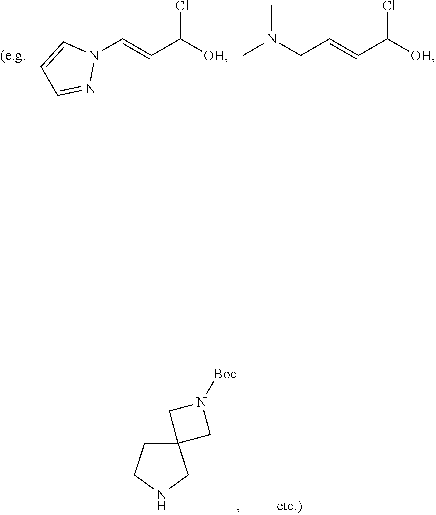

provided in the examples.

The present disclosure also provides a pharmaceutical composition, wherein the active ingredient of the pharmaceutical composition is selected from one or a combination of two or more of the compound above, or a tautomer, meso isomer, racemate, enantiomer, diastereomer or mixture thereof, or a pharmaceutically acceptable hydrate, solvate or salt thereof.

The present disclosure also provides use of the compound above, or a tautomer, meso isomer, racemate, enantiomer, diastereomer or mixture thereof, or a pharmaceutically acceptable hydrate, solvate or salt thereof or the pharmaceutical composition of above in the preparation of KRAS inhibitors and/or PI3K inhibitors.

Further, the KRAS inhibitors is selected from KRAS G12C inhibitors, KRAS G12V inhibitors, KRAS G12D inhibitors, KRAS G12S inhibitors, preferably KRAS G12C inhibitors; the PI3K inhibitors is PI3Kα inhibitors and/or PI3Kδ inhibitors.

The present disclosure also provides use of the compound above, or a tautomer, meso isomer, racemate, enantiomer, diastereomer or mixture thereof, or a pharmaceutically acceptable hydrate, solvate or salt thereof or the pharmaceutical composition of above in the preparation of a medicament for the treatment of a disease mediated by KRAS and/or PI3K.

The present disclosure provides the compound above, or a tautomer, meso isomer, racemate, enantiomer, diastereomer or mixture thereof, or a pharmaceutically acceptable hydrate, solvate or salt thereof or the pharmaceutical composition of above, for use in the treatment of a disease mediated by KRAS and/or PI3K.

The present disclosure provides a method in the treatment of a disease mediated by KRAS and/or PI3K, comprising administering to said individual an effective amount of a compound described above, or a tautomer, meso isomer, racemate, enantiomer, diastereomer or mixture thereof, or a pharmaceutically acceptable hydrate, solvate or salt thereof or the pharmaceutical composition of above.

Further, the present disclosure provides use of the compound above, or a tautomer, meso isomer, racemate, enantiomer, diastereomer or mixture thereof, or a pharmaceutically acceptable hydrate, solvate or salt thereof or the pharmaceutical composition of above in the preparation of medicament for the treatment of a disease mediated by one or more of KRAS G12C, PI3Kα, and PI3Kδ.

The present disclosure provides the compound above, or a tautomer, meso isomer, racemate, enantiomer, diastereomer or mixture thereof, or a pharmaceutically acceptable hydrate, solvate or salt thereof or the pharmaceutical composition of above, for use in the treatment of a disease mediated by one or more of KRAS G12C, PI3Kα, and PI3Kδ.

A method in the treatment of a disease mediated by one or more of KRAS G12C, PI3Kα, and PI3Kδ, comprising administering to said individual an effective amount of a compound described above, or a tautomer, meso isomer, racemate, enantiomer, diastereomer or mixture thereof, or a pharmaceutically acceptable hydrate, solvate or salt thereof or the pharmaceutical composition of above.

Further, the disease is cancer or an autoimmune disease.

Further, the cancer is selected from: non-small cell lung cancer, lung cancer, pancreatic cancer, ovarian cancer, bladder cancer, prostate cancer, chronic myelogenous leukemia, colorectal cancer, brain cancer, liver cancer, kidney cancer, stomach cancer, breast cancer, triple negative breast cancer, skin cancer, melanin Cancer, head and neck cancer, bone cancer, cervical cancer, pelvic cancer, vaginal cancer, oral cancer, lymphoma, blood cancer, esophageal cancer, urethra cancer, nasal cavity cancer.

The present disclosure also provides use of the compound above, or a tautomer, meso isomer, racemate, enantiomer, diastereomer or mixture thereof, or a pharmaceutically acceptable hydrate, solvate or salt thereof, or the pharmaceutical composition of above in the preparation of medicament for the treatment of a disease that is resistant to anticancer agents.

The present disclosure also provides the compound above, or a tautomer, meso isomer, racemate, enantiomer, diastereomer or mixture thereof, or a pharmaceutically acceptable hydrate, solvate or salt thereof or the pharmaceutical composition of above, for use in the treatment of a disease that is resistant to anticancer agents.

A method in the treatment of a disease that is resistant to anticancer agents, comprising administering to said individual an effective amount of a compound described above or a tautomer, meso isomer, racemate, enantiomer, diastereomer or mixture thereof, or a pharmaceutically acceptable hydrate, solvate or salt thereof or the pharmaceutical composition of above.

Further, the anticancer agent is selected from KRAS G12C inhibitors, KRAS G12V inhibitors, KRAS G12D inhibitors, KRAS G12S inhibitors, preferably KRAS G12C inhibitors;
further, the KRAS G12C inhibitors is selected from AMG-510, MRTX-849, preferably AMG-510.

The present disclosure also provides use of the compound above, or a tautomer, meso isomer, racemate, enantiomer, diastereomer or mixture thereof, or a pharmaceutically acceptable hydrate, solvate or salt thereof or the pharmaceutical composition of above in the preparation of medicament for the treatment of a disease causing overexpression of PI3K protein and/or KRAS G12C protein.

The present disclosure also provides use of the compound above, or a tautomer, meso isomer, racemate, enantiomer, diastereomer or mixture thereof, or a pharmaceutically acceptable hydrate, solvate or salt thereof or the pharmaceutical composition of above in the preparation of medicament for the treatment of a disease caused by overexpression of PI3K protein and/or KRAS G12C protein.

The present disclosure provides the compound above, or a tautomer, meso isomer, racemate, enantiomer, diastereomer or mixture thereof, or a pharmaceutically acceptable hydrate, solvate or salt thereof or the pharmaceutical composition of above, for use in the treatment of a disease caused by overexpression of PI3K protein and/or KRAS G12C protein.

The present disclosure provides a method in the treatment of a disease caused by overexpression of PI3K protein and/or KRAS G12C protein, including administering to said individual an effective amount of a compound described above, or a tautomer, meso isomer, racemate, enantiomer, diastereomer or mixture thereof, or a pharmaceutically acceptable hydrate, solvate or salt thereof or the pharmaceutical composition of above.

The pharmaceutical composition containing compound or a tautomer, meso isomer, racemate, enantiomer, diastereomer or mixture thereof, or a pharmaceutically acceptable hydrate, solvate or salt thereof in the present disclosure may contain pharmaceutically acceptable excipients.

“Pharmaceutically acceptable” as used herein refers to any substance that does not interfere with the effectiveness of the biological activity of the active ingredient and is non-toxic to the host to which it is administered.

Pharmaceutically acceptable excipients as claimed in the present disclosure are the general term for all additional materials in drugs other than the main drug. The excipients should have the following properties: (1) No toxic effects on the human body and almost no side effects; (2) Stable chemical properties, not easily affected by temperature, pH, storage time, etc.; (3) It has no incompatibility with the main drug and does not affect the efficacy and quality inspection of the main drug; (4) It does not interact with packaging materials. The excipients in the present disclosure include but are not limited to fillers (diluents), lubricants (glidants or anti-adhesive agents), dispersants, wetting agents, adhesives, regulators, solubilizers, antioxidants, and bacteriostatic agents, emulsifiers, disintegrants, etc. Adhesives include syrup, gum arabic, gelatin, sorbitol, tragacanth, cellulose and its derivatives (such as microcrystalline cellulose, sodium carboxymethylcellulose, ethylcellulose or hydroxypropylmethylcellulose, etc.), gelatin slurry, syrup, starch slurry or polyvinylpyrrolidone, etc.; Lubricants include micronized silica gel, magnesium stearate, talc, aluminum hydroxide, boronic acid, hydrogenated vegetable oil, polyethylene glycol, etc.; disintegrants include starch and its derivatives (such as sodium carboxymethyl starch, sodium starch glycolate, pregelatinized starch, modified starch, hydroxypropyl starch, corn starch, etc.), polyvinylpyrrolidone or microcrystalline cellulose, etc.; wetting agents include sodium lauryl sulfate, water or alcohol, etc.; antioxidants include sodium sulfite, sodium bisulfite, sodium metabisulfite, dibutylbenzoic acid, etc.; bacteriostatic agents include 0.5% phenol, 0.3% cresol, 0.5% chlorobutanol, etc.;

The term “pharmaceutically acceptable salt” refers to salts of compounds of the disclosure with acids or bases suitable for use as pharmaceuticals. The above acids and bases are generalized Lewis acids and bases. Suitable salt-forming acids include, but are not limited to: Hydrochloric acid, hydrobromic acid, hydrofluoric acid, sulfuric acid, nitric acid, phosphoric acid and other inorganic acids, formic acid, acetic acid, propionic acid, oxalic acid, malonic acid, succinic acid, fumaric acid, maleic acid, lactic acid, malic acid, tartaric acid, citric acid, picric acid, methanesulfonic acid, benzenesulfonic acid, benzenesulfonic acid and other organic acids; and acidic amino acids such as aspartic acid and glutamic acid.

The administration mode of the compound or pharmaceutical composition in the present disclosure is not particularly limited. Representative administration modes include (but are not limited to): oral, parenteral (intravenous, intramuscular or subcutaneous), and topical administration.

Solid dosage forms for oral administration include capsules, tablets, pills, powders and granules. In these solid dosage forms, the active compound is mixed with at least one conventional inert excipient (or carrier), such as sodium citrate or dicalcium phosphate, or with the following ingredients: (a) Fillers or compatibilizers, such as starch, lactose, sucrose, glucose, mannitol and silicic acid; (b) Adhesives such as hydroxymethylcellulose, alginate, gelatin, polyvinylpyrrolidone, sucrose and gum arabic; (c) Humectants such as glycerin; (d) Disintegrants such as agar, calcium carbonate, potato or tapioca starch, alginic acid, certain complex silicates, and sodium carbonate; (e) Retarders, such as paraffin; (f) Absorption accelerators, such as quaternary ammonium compounds; (g) Wetting agents, such as cetyl alcohol and glyceryl monostearate; (h) Adsorbent, such as kaolin; and (i) Lubricants, such as talc, calcium stearate, magnesium stearate, solid polyethylene glycol, sodium lauryl sulfate, or mixtures thereof. In capsules, tablets and pills, the dosage form may also contain buffering agents.

Solid dosage forms such as tablets, dragees, capsules, pills and granules may be prepared using coatings and shell materials such as enteric casings and other materials well known in the art. They may contain opacifying agents and the release of the active compound or compounds in such compositions may be in a delayed manner in a certain part of the digestive tract. Examples of embedding components that can be used are polymeric substances and waxy substances. If necessary, the active compounds can also be in microencapsulated form with one or more of the above-mentioned excipients.

Liquid dosage forms for oral administration include pharmaceutically acceptable emulsions, solutions, suspensions, syrups or tinctures. Liquid dosage forms may contain, in addition to the active compound, inert diluents conventionally employed in the art, such as water or other solvents, solubilizers and emulsifiers, for example, ethanol, isopropyl alcohol, ethyl carbonate, ethyl acetate, propylene glycol, 1,3-butanediol, dimethylformamide and oils, especially cottonseed oil, peanut oil, corn germ oil, olive oil, castor oil and sesame oil or mixtures of these substances, etc.

Besides these inert diluents, the compositions may also contain adjuvants such as wetting agents, emulsifying and suspending agents, sweetening, flavoring and perfuming agents.

Suspensions may contain, in addition to the active compound, suspending agents, for example, ethoxylated isostearyl alcohols, polyoxyethylene sorbitol and sorbitan esters, microcrystalline cellulose, aluminum methoxide and agar or mixtures of these substances and the like.

Compositions for parenteral injection may contain physiologically acceptable sterile aqueous or anhydrous solutions, dispersions, suspensions or emulsions, and sterile powders for reconstitution into sterile injectable solutions or dispersions. Suitable aqueous and non-aqueous carriers, diluents, solvents or excipients include water, ethanol, polyols and suitable mixtures thereof.

Dosage forms for topical administration of the compounds of this disclosure include ointments, powders, patches, sprays and inhalants. The active ingredient is mixed under sterile conditions with a physiologically acceptable carrier and any preservatives, buffers, or propellants that may be required.

The compounds in the present disclosure may also be used in injectable preparations. Wherein, the injection is selected from liquid injection (water injection), sterile powder for injection (powder injection) or injection tablets (Refers to the molded tablets or machine-pressed tablets made by aseptic methods, which are dissolved in water for injection for subcutaneous or intramuscular injection).

Wherein, in addition to the above compounds, the injection powder also contains at least excipients. The excipients mentioned in the present disclosure are ingredients intentionally added to the medicine, and they should not have pharmacological properties in the amount used. However, excipients may aid in processing, dissolution or dissolution of the drug, delivery through a targeted route of administration, or aid in stability. ‘optionally substituted by one or more’ means it can be substituted by one or more specified substituents, or it can be unsubstituted; if ‘more’ in ‘one or more’ is not limited, the minimum value is 2, and the maximum value is the possible number of the substitution site of substituted groups.

If a substituent is described as being ‘independently selected from’ a group, each substituent is selected independently of the other. Thus, each substituent may be the same as or different from another (or other) substituent.

‘Substituted’ means that a hydrogen atom in a molecule is replaced by a different group.

‘Membered’ refers to the number of skeleton atoms constituting the ring.

The ‘bond’ in the present disclosure means that there is only one bond, which can also be understood as ‘none’.

As used herein, alkyl as optional substituents for R^{g} and R¹¹ include deuterated alkyl. ‘Alkyl’ refers to an aliphatic hydrocarbon group and refers to a saturated hydrocarbon group. The alkyl may be a straight chain alkyl group or a branched chain alkyl group. Typical alkyl groups include, but are not limited to, methyl, ethyl, propyl, isopropyl, butyl, isobutyl, tert-butyl, pentyl, hexyl, etc.

As used in the present disclosure, C1˜Cn includes C1˜C2, C1˜C3 . . . C1˜Cn with n as an integer greater than one. The prefix used as a substituent indicates the minimum and maximum number of carbon atoms in the substituent. For example, ‘C1˜C6 alkyl’ refers to a straight-chain or branched alkyl group containing 1 to 6 carbon atoms.

‘Heteroalkyl’ refers to an alkyl group containing heteroatoms.

‘Ring’ refers to any covalently closed structure, including, for example, carbocycles (e.g., aryl or cycloalkyl), heterocycles (e.g., heteroaryl or heterocycloalkyl), aryls (e.g., aryl or heteroaryl), non-aromatic groups (such as cycloalkyl or heterocycloalkyl). The ‘ring’ in the present disclosure can be a monocyclic ring, as well as a polycyclic ring and also parallel ring, spirocyclic ring or bridged cyclic ring

‘Cycloalkyl’ refers to a saturated cyclic hydrocarbon substituent.

‘Cycloalkenyl’ refers to a cyclic substituent containing at least one carbon-carbon double bond in the ring skeleton.

‘Heterocycloalkyl’ refers to a saturated ring substituent containing heteroatoms in the ring skeleton.

‘Nitrogen-containing heterocycloalkyl’ refers to a cycloalkyl group containing nitrogen atoms in the ring skeleton, and the same applies to other similar situations.

‘Nitrogen-containing monoheterocycloalkyl’ refers to nitrogen-containing heterocycloalkyl with a monocyclic structure, and the same applies to other similar situations.

‘Alicyclyl’ refers to a cyclic substituent without aromaticity, which may be a cycloalkyl group, a cycloalkenyl group or an alicyclic heterocyclic group.

‘Aliphatic heterocyclyl’ refers to a substituent group formed by a non-aromatic heterocyclic compound containing at least one heteroatom on the ring skeleton, and ‘aliphatic heterocyclyl’ includes ‘heterocycloalkyl’.

Typical aliphatic heterocyclyl include but are not limited to:

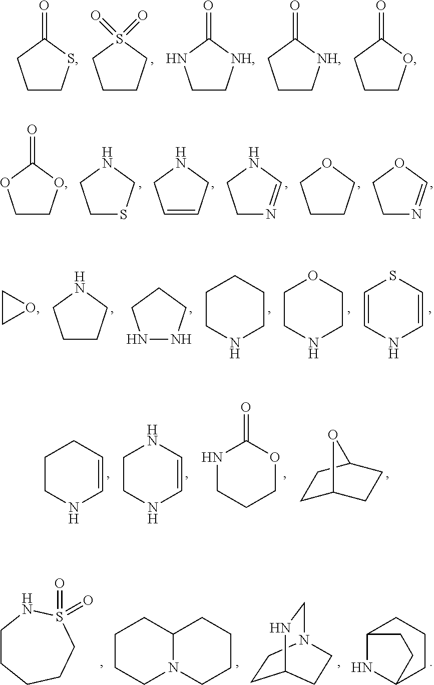

‘Aryl’ refers to an aromatic monocyclic or polycyclic group whose planar ring has a delocalized π electron system and contains 4n+2 π electrons, where n is an integer; typical aryl include but not limited to phenyl, naphthyl, phenanthrenyl, anthracenyl, fluorenyl and indenyl, etc.

‘Heteroaryl’ refers to a monocyclic or polycyclic group containing heteroatoms and having aromatic properties. Typical heteroaryl include but are not limited to:

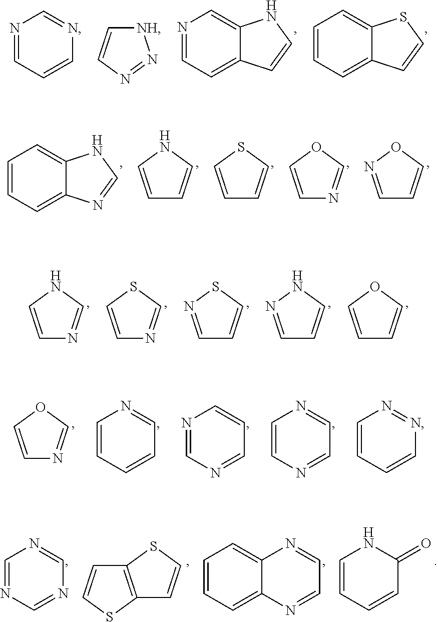

As used herein, the alkyl, cycloalkyl, cycloalkenyl, aliphatic cycloalkyl, heterocyclyl, heterocycloalkyl, aryl and heteroaryl, etc. can be unsubstituted alkyl, cycloalkyl, cycloalkenyl, aliphatic cycloalkyl, heterocyclyl, heterocycloalkyl, aryl and heteroaryl, etc., as well as alkyl, cycloalkyl, cycloalkenyl, aliphatic cycloalkyl, heterocyclyl, heterocycloalkyl, aryl and heteroaryl, etc.

As used herein, unless otherwise specified, ‘substituted’ means that the mentioned group may be substituted by one or more additional groups, each of which is independently selected from substituent groups common in the art, such as halogen, cyano, hydroxy, amino, carboxyl, alkyl, alkoxy, alkylamino, alkylthio, haloalkyl, cycloalkyl, heterocycloalkyl, aryl, heteroaryl, etc.

‘Alkoxy’ is —O-alkyl.

‘Alkylamino’ is —NH-alkyl or —N-(alkyl)₂

‘Alkoxy’ is —S-alkyl.

‘Halogen’ or ‘Halo’ is F, Cl, Br, or I.

‘Cyano’ is —CN.

‘Amino’ is —NH₂.

‘Hydroxy’ is —OH.

‘(O)’ is ═O, as —C(O)R¹³ is

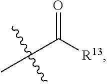

and the same applies to other similar situations.

C═O is

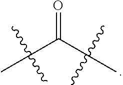 [SMILES: *C(*)=O]

The structural formula of pyridine is

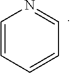

The structural formula of thiophene is

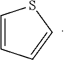

The structural formula of pyrrole is

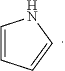

The structural formula of 1H-1,2,3-triazole is

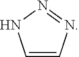

The structural formula of oxazole is

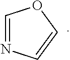

As used herein, an ‘individual’ includes a human or non-human animal. Exemplary human subjects include human subjects (referred to as patients) suffering from a disease, such as those described herein, or normal subjects. ‘Non-human animals’ include all vertebrate animals, such as non-mammals and mammals, such as non-human primates, domestic animals and/or domesticated animals.

As used herein, an ‘effective amount’ refers to an amount of a compound that, when administered, alleviates to a certain extent one or more symptoms of the disease being treated. Dosage regimens can be adjusted to provide the best desired response.

The beneficial effects of the present disclosure are:
(1) The present disclosure provides a series of compounds with obvious inhibitory effects on KRAS and PI3K proteins, provides new solutions for the treatment of diseases such as cancer with KRAS or PI3K as the target, and can be used to prepare medicines for treating related diseases, with broad application prospects.
(2) The compound of the present disclosure has a strong inhibitory effect on AMG-510-resistant cancer cells, overcomes the resistance problem caused by simple G12C inhibitors, and is expected to prolong the survival period of patients.

### DETAILED DESCRIPTION OF THE EMBODIMENTS

The technical solution of the present disclosure is clearly and completely described below. Obviously, the described embodiments are some of the embodiments of the present disclosure, rather than all the embodiments. Based on the embodiments of the present disclosure, all other embodiments obtained by those of ordinary skill in the art without creative efforts fall within the scope of protection of the present disclosure.

The structure of the compound of the present disclosure is determined by Nuclear Magnetic Resonance (NMR) or/and Liquid Chromatography-Mass Spectrometry (LC-MS). Chemical shifts (δ) for NMR are given in units of parts per million (ppm). NMR was measured using an AVANCE NEO 400 MHz Bruker instrument. The solvents used for the determination were deuterated dimethyl sulfoxide (DMSO-d₆), deuterated chloroform (CDCl₃), and deuterated methanol (CD₃OD). The internal standard was tetramethylsilane (TMS). MS was measured using an ISQ-EC Thermo Fisher LC-MS instrument. Prep-HPLC is a GX-281 Gilson chromatograph. The separation methods used are: (Method 1) Sun Fire Prep C18 OBDTM 5 μm, 30×150 mm Column, 0.04% HCl aqueous solution/acetonitrile; (Method 2) Xbridge Prep C18 OBDTM 5 μm, 30×150 mm Column, 10 mM NH₄HCO aqueous solution/acetonitrile.

The starting materials in the examples of the present disclosure are known and can be purchased on the market, or can be synthesized according to methods known in the art.

The solvents used in the present disclosure, unless otherwise specified, are commercially available.

The reaction temperature in the examples, unless otherwise specified, is room temperature, which is 20° C. to 30° C.

The chemical abbreviations involved in the present disclosure have the following meanings:
TFA: trifluoroacetic acid
DMF: N,N-dimethylformamide
DMSO: dimethyl sulfoxide
THF: tetrahydrofuran
HOBT: 1-hydroxybenzotriazole
EDCT: 1-(3-dimethylaminopropyl)-3-ethylcarbodiimide hydrochloride
DIPEA: N,N-diisopropylethylamine
HATU: O-(7-azabenzotriazol-1-yl)-N,N,N′,N′-tetramethylurea
T₃P: 1-propylphosphonic anhydride
BINAP: 1,1′-binaphthyl-2,2′-bisdiphenylphosphine
Prep-HPLC: preparative High-Performance Liquid Chromatography

### Example 1

### Preparation of N-(5-(4-(4-acryloylpiperazin-1-yl)quinazolin-6-yl)-2-methoxypyridin-3-yl)-2,4-difluorobenzenesulfonamide

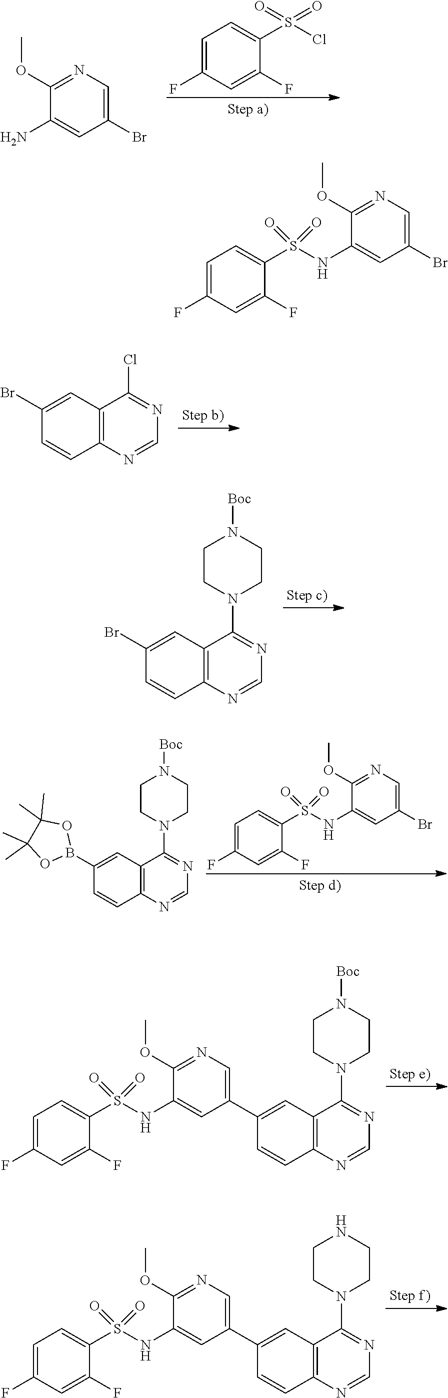

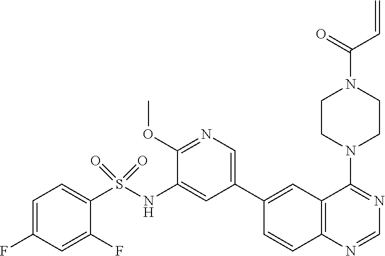 [SMILES: C=CC(=O)N1CCN(c2ncnc3ccc(-c4cnc(OC)c(NS(=O)(=O)c5ccc(F)cc5F)c4)cc23)CC1]

### Step a): Preparation of N-(5-bromo-2-methoxypyridin-3-yl)-2,4-difluorobenznesulfonamide

5-Bromo-2-methoxypyridin-3-amine (2.0 g 9.850 mmol), 2,4-difluorobenzenesulfonyl chloride (2.3 g, 10.835 mmol), 4-dimethylaminopyridine (60 mg, 0.493 mmol) and pyridine (1.2 g, 14.775 mmol) were dissolved in dichloromethane (20 mL). After the addition was completed, the reaction mixture was stirred at room temperature for 18 h. Upon completion of the reaction, the reaction was quenched by adding water (50 mL), and the reaction mixture was extracted with dichloromethane (100 mL×2). The organic phases were combined, dried over anhydrous sodium sulfate and filtered. The filtrate was concentrated in vacuo. The crude product obtained was purified by silica gel column chromatography (Eluent: petroleum ether/ethyl acetate=3/1 to 1/1). N-(5-bromo-2-methoxypyridin-3-yl)-2,4-difluorobenzene sulfonamide was obtained, yield: 37.3% ESI-MS(m/z): 381.0 [M+H]⁺.

### Step b): Preparation of tert-butyl 4-(6-bromoquinazolin-4-yl)piperazine-1-carboxylate

6-Bromo-4-chloroquinazoline (1.9 g, 7.820 mmol) and tert-butyl piperazine-1-carboxylate (1.8 g, 9.380 mmol) were dissolved in dimethyl sulfoxide (30 mL), with triethyl amine (2.4 g, 23.470 mmol) added. After the addition was completed, the reaction mixture was heated to 60° C. and stirred for 6 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated in vacuo. The crude product obtained was purified by silica gel column chromatography (Eluent: dichloromethane/methanol=50/1 to 10/1) tert-butyl 4-(6-bromoquinazolin-4-ol)piperazine-1-carboxylate was obtained, yield: 65.4%. ESI-MS(m/z): 393.1 [M+H]⁺.

### Step c): Preparation of tert-butyl 4-(6-(4,4,5,5-tetramethyl-1,3,2-dioxaborolan-2-yl)quinazolin-4-yl)piperazine-4-carboxylate

tert-butyl 4-(6-bromoquinazolin-4-yl)piperazine-1-carboxylate (2.0 g, 5.10 mmol), bis(pinacolato)diboron (1.9 g, 7.640 mmol) Pd(dppf)Cl₂ (373.8 g 0.510 mmol) and potassium acetate (1.5 g, 15.310 mmol) were dissolved in dioxane (30 mL). After the addition was completed, in the presense of protective nitrogen, the reaction mixture was kept at 110° C. and stirred for 4 h. Upon completion of the reaction, the reaction mixture was filtered, and the filtrate was concentrated under reduced pressure to provide the crude residue. The crude product obtained was purified by silica gel column chromatography (Eluent: dichloromethane/methanol=50/1 to 10/1), tert-butyl 4-(6-(4,4,5,5-tetramethyl-1,3,2-dioxaborolan-2-yl)quinazolin-4-yl)piperazine-1-carboxylate was obtained, yield: 62.5%; ESI-MS(m/7): 441.3 [M+H]⁺.

### Step d): Preparation of tert-butyl 4-(6-(5-((2,4-difluorophenyl)sulfonamido)-6-methoxypyridin-3-yl)quinazolin-4-yl)piperazine-1-carboxylate

N-(5-bromo-2-methoxypyridin-3-yl)-2,4-difluorobenzenesulfonamide (300 mg, 0.790 mmol), tert-butyl 4-(6-(4,4,5,5-tetramethyl-1,3,2-dioxaborolan-2-yl)quinazolin-4-yl)piperazine-1-carboxylate (698 mg, 1.580 mmol), Pd(dppf)cl₂ (58 mg, 0.080 mmol) and cesium carbonate (779 mg, 2.390 mmol) were dissolved in dioxane/water (4:1, 10 mL). After the addition was completed, in the presense of protective nitrogen, the reaction mixture was kept at 110° C. and stirred for 4 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=1/1 to 1/3), tert-butyl 4-(6-(5-((2,4-difluorophenyl)sulfonamido)-6-methoxypyridin-3-yl)quinazolin-4-yl)piperazine-1-carboxylate was obtained, yield: 82.5%. ESI-MS(m/z): 613.1 [M+H]⁺.

### Step e): Preparation of 2,4-difluoro-N-(2-methoxy-5-(4-(piperazin-1-yl)quinazolin-6-yl)pyridin-3-yl)benzenesulfonamide

tert-butyl 4-(6-(5-((2,4-difluorophenyl)sulfonamido)-6-methoxypyridin-3-yl)quinazolin-4-yl)piperazine-1-carboxylate (400 mg, 0.650 mmol) were dissolved in dichloromethane (8 mL), under the ice bath condition, trifluoroacetic acid (2 mL) was slowly added dropwise. After the addition was completed, the reaction mixture was stirred at room temperature for 1 h. Upon completion of the reaction, the reaction mixture was concentrated under reduced pressure, 2,4-difluoro-N-(2-methoxy-5-(4-(piperazin-1-yl)quinazolin-6-yl)pyridin-3-yl)benzenesulfonamide was obtained. ESI-MS(m/z): 513.1 [M+H]⁺.

### Step f): Preparation of N-(5-(4-(4-acryloylpiperazin-1-yl)quinazolin-6-yl)-2-methoxypyridin-3-yl)-2,4-difluorobenzenesulfonamide

To a solution of 2,4-difluoro-N-(2-methoxy-5-(4-(piperazin-1-yl)quinazolin-6-yl)pyridin-3-yl)benzenesulfonamide (170 mg, 0.330 mmol), HOBT (54 mg, 0.400 mmol), and EDCI (76 mg, 0.400 mmol) in dichloromethane (10 mL) was added DIEA (215 mg, 1.650 mmol) and acrylic (36 mg, 0.500 mmol) slowly at −78° C. After the addition was completed, the reaction mixture was kept at −78° C. and stirred for 1 h. Upon completion of the reaction, the reaction was quenched by adding water (50 mL), and the reaction mixture was extracted with dichloromethane (100 mL×2). The organic phases were combined, dried over anhydrous sodium sulfate and filtered. The filtrate was concentrated under reduced pressure. The crude product obtained was purified by Prep-HPLC (Method 2), N-(5-(4-(4-acryloylpiperazin-1-yl)quinazolin-6-yl)-2-methoxypyridin-3-yl)-2,4-difluorobenzenesulfonamide was obtained, yield: 10.2%; ¹H NMR (400 MHz, DMSO-d₆) δ 10.34 (s, 1H), 8.66 (s, 1H), 8.47 (s, 1H), 8.09-8.07 (m, 2H), 8.02 (s, 1H), 7.93-7.91 (m, 1H), 7.80-7.77 (m, 1H), 7.60-7.55 (m, 1H), 7.24-7.20 (m, 1H), 6.87-6.81 (m, 1H), 6.19 (d, J=8.0 Hz, 1H), 5.76-5.73 (m, 1H), 3.90-3.79 (m, 8H), 3.69 (s, 3H); ESI-MS(m/z): 567.0 [M+H]⁺.

### Example 2

### N-(5-(4-((1-acryloylpiperidin-4-yl)amino)pyrido[3,2-d]pyrimidin-6-yl)-2-chloropyridin-3-yl)-2,4-difluorobenzenesulfonamide

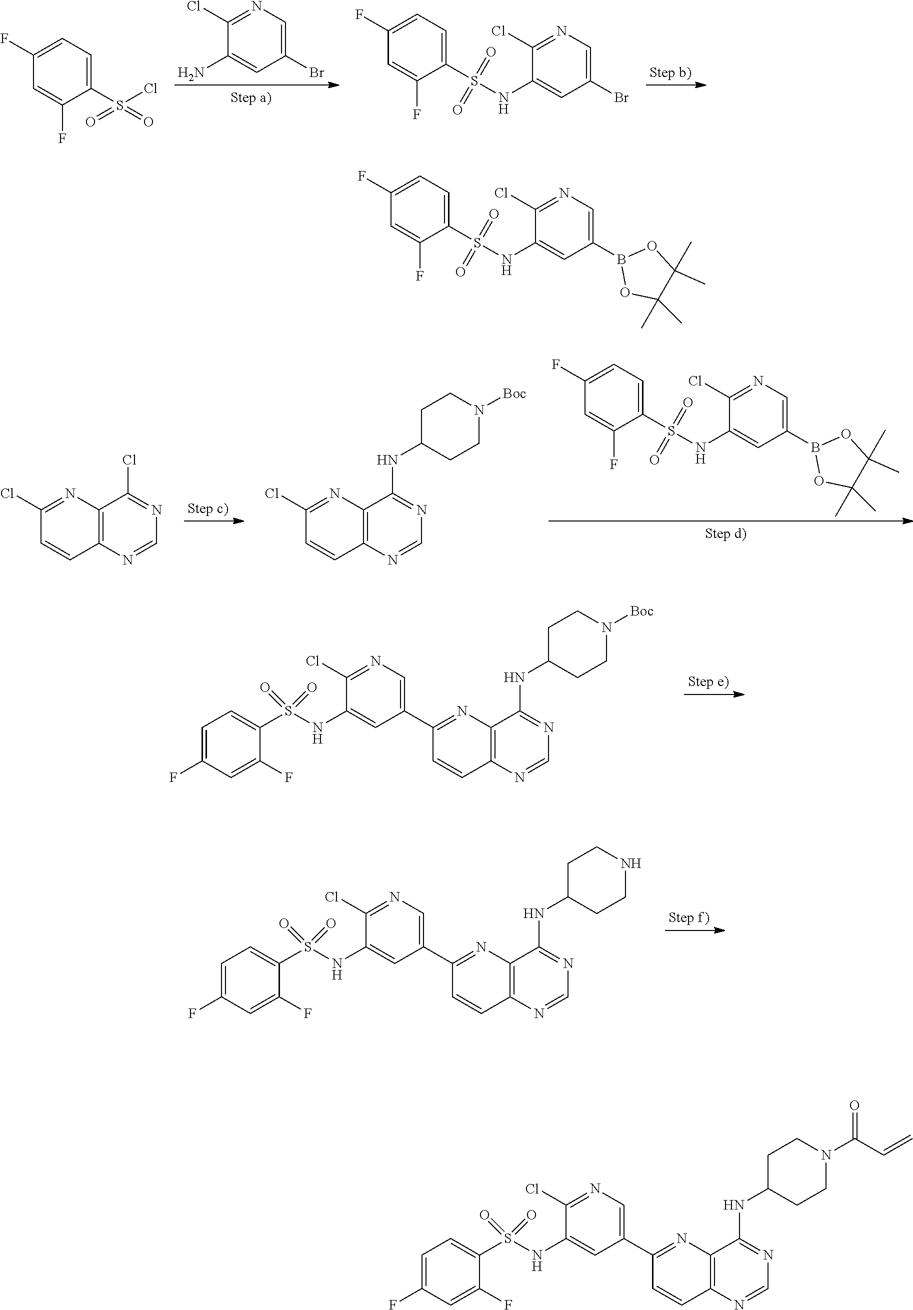

### Step a): Preparation of N-(5-bromo-2-chloropyridin-3-yl)-2,4-difluorobenzenesulfonamide

5-Bromo-2-chloropyridin-3-amine (2.0 g, 9.640 mmol), 2,4-difluorobenzenesulfonyl chloride (2.3 g, 10.819 mmol) were added to pyridine (20 mL). After the addition was completed, the reaction mixture was heated to 80′C and stirred for 18 h. Upon completion of the reaction, the reaction was quenched by adding water (50 mL), and the reaction mixture was extracted with dichloromethane (100 mL×2). The organic phases were combined, washed with saturated brine (50 mL×1), dried over anhydrous sodium sulfate and filtered. The filtrate was concentrated under reduced pressure. The crude product obtained was purified by silica gel column chromatography (Eluent: dichloromethane/methanol=100/1 to 50/1), N-(5-bromo-2-chloropyridin-3-yl)-2,4-difluorobenzenesulfonamide was obtained, yield: 31.2%; ESI-MS(m/z): 384.9 [M+H]⁺.

### Step b): Preparation of N-(2-chloro-5-(4,4,5,5-tetramethyl-1,3,2-dioxaborolan-2-yl)pyridin-3-yl)-2,4-difluorobenzenesulfonamide

N-(5-bromo-2-chloropyridin-3-yl)-2,4-difluorobenzenesulfonamide (450 mg, 1.173 mmol), bis(pinacolato)diboron (298 mg, 1.173 mmol), Pd(dppf)Cl₂ (172 mg, 0.235 mmol) and potassium acetate (345 mg, 3.519 mmol) were dissolved in dioxane (10 mL). After the addition was completed, in the presense of protective nitrogen, the reaction mixture was heated to 100° C. and stirred for 4 h. Upon completion of the reaction, the reaction was quenched by adding water (30 mL). The reaction mixture was extracted with ethyl acetate (50 mL×2). The organic phases were combined, washed with saturated brine (50 mL×1), dried over anhydrous sodium sulfate and filtered. The filtrate was concentrated through vacuum concentration. The crude product obtained was purified by silica gel column chromatography (Eluent: petroleum ether/ethyl acetate=10/1 to 5/1), N-(2-chloro-5-(4,4,5,5-tetramethyl-1,3,2-dioxaborolan-2-yl)pyridin-3-yl)-2,4-difluorobenzenesulfonamide was obtained, yield: 58.8%; ESI-MS(m/z): 431.1 [M+H]⁺.

### Step c): Preparation of tert-butyl 4-((6-chloropyrido[3,2-d]pyrimidin-4-yl)amino)piperidine-1-carboxylate

4,6-Dichloropyrido[3,2-d]pyrimidine (500 mg, 2.500 mmol), 4-aminopiperidine-1-carboxylic acid tert-butyl ester (551 mg, 2.750 mmol) were dissolved in dimethyl sulfoxide (50 mL), with N,N-diisopropylethylamine (969 mg, 7.500 mmol) added. After the addition was completed, the reaction mixture was heated to 55° C. and stirred for 2 h. Upon completion of the reaction, the reaction was quenched by adding water (50 mL). The reaction mixture was extracted with ethyl acetate (100 mL/2). The organic phases were combined and washed with saturated brine (50 mL 1), dried over anhydrous sodium sulfate and filtered. The filtrate was concentrated under reduced pressure. The crude product obtained was purified by silica gel column chromatography (Eluent: petroleum ether/ethyl acetate=10/1 to 2/1), tert-butyl 4-((6-chloropyrido[3,2-d]pyrimidin-4-yl)amino)piperidine-1-carboxylate was obtained, yield: 87.9%; ESI-MS(m/z): 364.2 [M+H]⁺.

### Step d): Preparation of tert-butyl 4-((6-(6-chloro-5-((2,4-difluorophenyl)sulfonamido)pyridin-3-yl)pyrido[3,2-d]pyrimidin-4-yl)amino)piperidine-1-carboxylate

tert-butyl 4-((6-chloropyrido[3,2-d]pyrimidin-4-yl)amino)piperidine-1-carboxylate (420 mg, 1.154 mmol), N-(2-chloro-5-(4,4,5,5-tetramethyl-1,3,2-dioxaborolan-2-yl)pyridin-3-yl)-2,4-difluorobenzenesulfonamide (497 mg, 1.154 mmol), Pd(dppf)Cl₂ (169 mg, 0.231 mmol) and cesium carbonate (1.1 g, 3.376 mmol) were added to dioxane/water mixed solvent (10 mL, v/v=4:1). After the addition was completed, in the presense of protective nitrogen, the reaction mixture was heated to 100° C. and stirred for 4 h. Upon completion of the reaction, the reaction was quenched by adding water (50 mL). The reaction mixture was extracted with ethyl acetate (100 mL×2). The organic phases were combined and washed with saturated brine (50 mL×1), dried over anhydrous sodium sulfate and filtered. The filtrate was concentrated under reduced pressure. The crude product obtained was purified by silica gel column chromatography (Eluent: dichloromethane/methanol=50/1 to 30/1), tert-butyl 4-((6-(6-chloro-5-((2,4-difluorophenyl)sulfonamido)pyridin-3-yl)pyrido[3,2-d]pyrimidin-4-yl)amino)piperidine-1-carboxylate was obtained, yield: 37.0%; ESI-MS(m/z): 632.2 [M+H]⁺.

### Step e): N-(2-chloro-5-(4-(piperidin-4-ylamino)pyrido[3,2-d]pyrimidin-6-yl)pyridin-3-yl)-2,4-difluorobenzenesulfonamide

tert-butyl 4-((6-(6-chloro-5-((2,4-difluorophenyl)sulfonamido)pyridin-3-yl)pyrido[3,2-d]pyrimidin-4-yl)amino)piperidine-1-carboxylate (70 mg, 0.132 mmol) was dissolved in hydrogen chloride dioxane solution (10 mL, 4M), and the reaction mixture was stirred at room temperature for 1 h. Upon completion of the reaction, the reaction mixture was concentrated under reduced pressure, quenched by adding saturated sodium bicarbonate solution (20 mL), extracted with ethyl acetate (50 mL×2). The organic phases were combined and washed with saturated brine (50 mL×1), dried over anhydrous sodium sulfate and filtered. The filtrate was concentrated under reduced pressure, N-(2-chloro-5-(4-(piperidin-4-ylamino)pyrido[3,2-d]pyrimidin-6-yl)pyridin-3-yl)-2,4-difluorobenzenesulfonamide was obtained. The product can be used directly in the next reaction without purification. ESI-MS(m/z): 532.2 [M+H]⁺.

### Step f): N-(5-(4-((1-acryloylpiperidin-4-yl)amino)pyrido[3,2-d]pyrimidin-6-yl)-2-chloropyridin-3-yl)-2,4-difluorobenzenesulfonamide

N-(2-chloro-5-(4-(piperidin-4-ylamino)pyrido[3,2-d]pyrimidin-6-yl)pyridin-3-yl)-2,4-difluorobenzenesulfonamide (50 mg, 0.094 mmol), 1-hydroxybenzotriazole (15 mg, 0.113 mmol), 1-ethyl-(3-dimethylaminopropyl)carbodiimide hydrochloride (22 mg, 0.113 mmol) were dissolved in dichloromethane (5 mL), with N,N-diisopropylethylamine (36 mg, 0.282 mmol) and acrylic acid (8 mg, 0.113 mmol) slowly added under −78° C. After the addition was completed, the reaction mixture was kept at −78° C. and stirred for 1 h. Upon completion of the reaction, the reaction was quenched by adding water (20 mL), and the reaction mixture was extracted with dichloromethane (50 mL). The organic phases were combined, concentrated under reduced pressure. The crude product obtained was purified by Prep-HPLC (Method 2), N-(5-(4-((1-acryloylpiperidin-4-yl)amino)pyrido[3,2-d]pyrimidin-6-yl)-2-chloropyridin-3-yl)-2,4-difluorobenzenesulfonamide was obtained, yield: 15.8%; ¹H NMR (400 MHz, DMSO-d₆) δ 9.25-9.05 (m, 1H), 8.53-8.51 (m, 2H), 8.33-8.19 (m, 3H), 7.85-7.79 (m, 1H), 7.46-7.31 (m, 1H), 7.26-7.18 (m, 1H), 6.92-6.85 (m, 1H), 6.18-6.13 (m, 1H), 5.72-5.69 (m, 1H), 4.54-4.41 (m, 2H), 4.24-4.11 (m, 2H), 2.85-2.67 (m, 1H), 2.01-1.97 (m, 2H), 1.73-1.67 (m, 2H); ESI-MS(m/z): 586.0 [M+H]⁺.

### Example 3

### Preparation of N-(5-(4-(4-acryloylpiperazin-1-yl)-7-chloroquinazolin-6-yl)-2-methoxypyridin-3-yl)methanesulfonamide

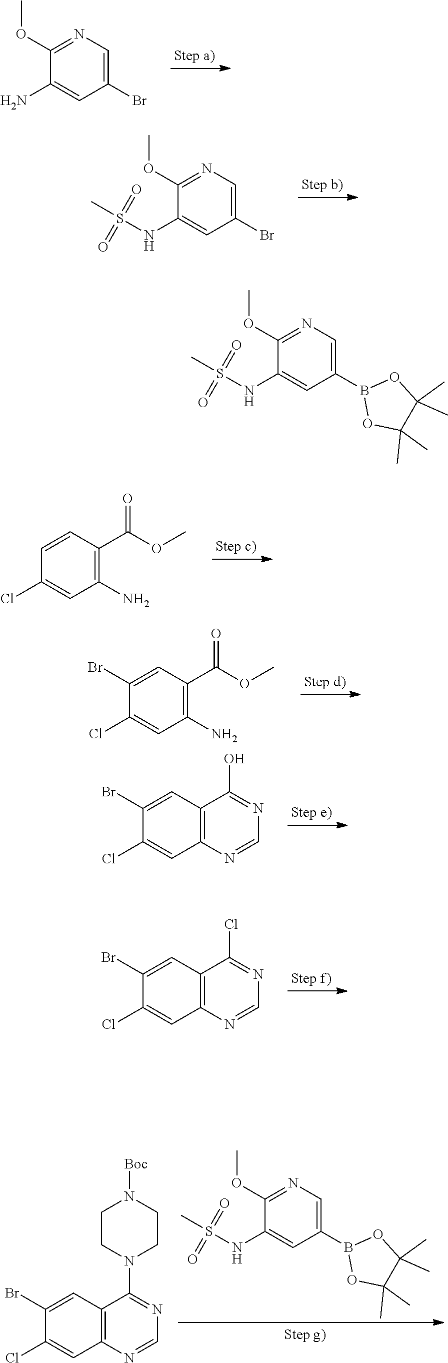

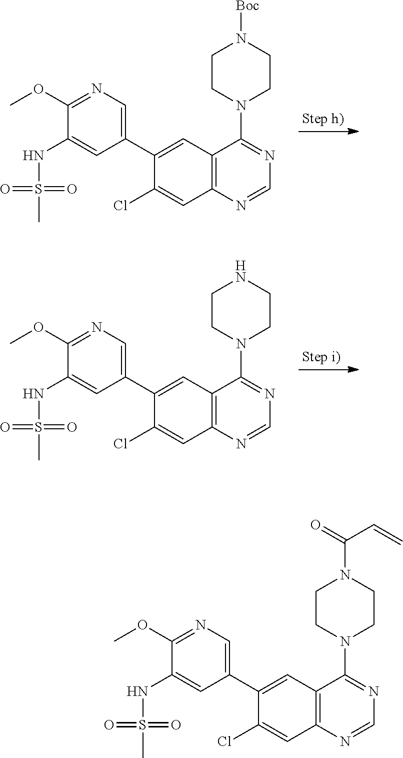 [SMILES: COc1ncc(-c2cc3c(N4CCN(C(=O)OC(C)(C)C)CC4)ncnc3cc2Cl)cc1NS(C)(=O)=O ; COc1ncc(-c2cc3c(N4CCNCC4)ncnc3cc2Cl)cc1NS(C)(=O)=O ; C=CC(=O)N1CCN(c2ncnc3cc(Cl)c(-c4cnc(OC)c(NS(C)(=O)=O)c4)cc23)CC1]

### Step a): Preparation of N-(5-bromo-2-methoxypyridin-3-yl)methanesulfonamide

Methanesulfonyl chloride (2.3 g, 20.087 mmol) and pyridine (4.8 g, 60.261 mmol) were dissolved in acetonitrile (40 mL), with 5-bromo-2-methoxypyridin-3-amine (3.4 g, 16.739 mmol) added. After the addition was completed, the reaction mixture was stirred at room temperature for 6 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=5/1 to 1:1). N-(5-bromo-2-methoxypyridin-3-yl)methanesulfonamide was obtained, yield: 42.3%; ESI-MS(m/z): 280.9 [M+H]⁺.

### Step b): Preparation of N-(2-methoxy-5-(4,4,5,5-tetramethyl-1,3,2-dioxaborolan-2-yl)pyridin-3-yl)methanesulfonamide

N-(5-bromo-2-methoxypyridin-3-yl)methanesulfonamide (500) mg, 1.779 mmol), bis(pinacolato)diboron (677.93 mg, 2.669 mmol), Pd(dppf)Cl₂ (130.25 mug, 0.178 mmol) and potassium acetate (523.13 mug, 5.339 mmol) were added to dioxane (15 mL). After the addition was completed, in the presense of protective nitrogen, the reaction mixture was kept at 110° C. and stirred for 4 h. Upon completion of the reaction, the reaction mixture was filtered. The filtrate was concentrated under reduced pressure. The crude product obtained was purified by silica gel column chromatography (Eluent: petroleum ether/ethyl acetate=5/1 to 1:1). N-(2-methoxy-5-(4,4,5,5-tetramethyl-1,3,2-dioxaborolan-2-yl)pyridin-3-yl)methanesulfonamide was obtained, yield: 85.7%; ESI-MS(m/z): 329.0 [M+H]⁺.

### Step c): Preparation of methyl 2-amino-5-bromo-4-chlorobenzoate

Methyl 2-amino-4-chlorobenzoate (5.0 g, 26.940 mmol) was dissolved in N,N-dimethylformamide (60 mL). Under the ice bath condition, N-bromosuccinimide (4.8 g, 26.940 mmol) was added to the system. After the addition was completed, the reaction mixture was stirred at room temperature overnight. Upon completion of the reaction, the reaction was quenched by adding water (80 mL), and the reaction mixture was extracted with dichloromethane (200 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=20/1 to 1/1). methyl 2-amino-5-bromo-4-chlorobenzoate was obtained, yield: 91.2%; ESI-MS(m/z): 265.9 [M+H]⁺.

### Step d): Preparation of 6-bromo-7-chloroquinazolin-4-ol

Methyl 2-amino-5-bromo-4-chlorobenzoate (1.0 g, 3.781 mmol) was added to methylamide (10 mL). The reaction mixture was heated to 200° C. and stirred for 3 h. Upon completion of the reaction, the reaction mixture was cooled to room temperature, quenched by adding water (50 mL). The solution was filtered after solid precipitation appeared. The filter cake was dried to obtain 6-Bromo-7-chloroquinazolin-4-ol, yield: 67.2%; ESI-MS(m/z): 260.9 [M+H]⁺.

### Step e): Preparation of 6-bromo-4,7-dichloroquinazoline

6-bromo-7-chloroquinazolin-4-ol (660 mg, 2.543 mmol) and N,N-diisopropylethylamine (0.5 mL) were dissolved in thionyl chloride (10 mL). The reaction mixture was heated to 100° C. and stirred for 3 h. Upon completion of the reaction, the reaction mixture was concentrated under reduced pressure, the reaction was quenched by adding water (30 mL). The reaction mixture was extracted with ethyl acetate (60 mL×2). The organic phases were combined, dried over anhydrous sodium sulfate and then filtered. The filtrate was concentrated under reduced pressure. The crude product obtained was purified by silica gel column chromatography (Eluent: petroleum ether/ethyl acetate=30/1 to 10/1), 6-Bromo-4,7-dichloroquinazoline was obtained, yield: 38.2%; ESI-MS(m/z): 278.9[M+H]⁺.

### Step f): Preparation of tert-butyl 4-(6-bromo-7-chloroquinazolin-4-yl)piperazine-1-carboxylate

6-Bromo-4,7-dichloroquinazoline (270 mg, 0.972 mmol), 1-tert-butoxycarbonylpiperazine (271.5 mg, 1.458 mmol) and triethylamine (294.9 mg, 2.916 mmol) were added to dichloromethane (5 mL) under the condition of stirring. The reaction mixture was stirred at room temperature for 1 h. Upon completion of the reaction, the reaction was quenched by adding water (20 mL). The reaction mixture was extracted with ethyl acetate (50 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=10/1 to 3/1). Tert-butyl 4-(6-bromo-7-chloroquinazolin-4-yl)piperazine-1-carboxylate was obtained, yield: 67.3%; ESI-MS(m/z): 429.0 [M+H]⁺.

### Step g): Preparation of tert-butyl 4-(7-chloro-6-(6-methoxy-5-(methylsulfonamido)pyridin-3-yl)quinazolin-4-yl)piperazine-1-carboxylate

Tert-butyl 4-(6-bromo-7-chloroquinazolin-4-yl)piperazine-1-carboxylate (280 mg, 0.655 mmol), N-(2-methoxy-5-(4,4,5,5-tetramethyl-1,3,2-dioxaborolan-2-yl)pyridin-3-yl)methanesulfonamide (429.7 mg, 1.310 mmol). Pd(dppf)Cl₂ (47.9 mg, 0.065 mmol) and potassium carbonate (181 mg, 1.310 mmol) were added to dioxane (4 mL) and water (0.8 mL) under the condition of stirring. After the addition was completed, in the presense of protective nitrogen, the reaction mixture was heated to reflux and stirred for 2 h. Upon completion of the reaction, the reaction was quenched by adding water (20 mL), and the reaction mixture was extracted with dichloromethane (50 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=3/1 to 1/1), 4-(7-chloro-6-(6-methoxy-5-(methylsulfonamido)pyridin-3-yl)quinazolin-4-yl)piperazine-1-carboxylate was obtained, yield: 61.2%; ESI-MS(m/z): 549.2 [M+H]⁺.

### Step h): Preparation of N-(5-(7-chloro-4-(piperazin-1-yl)quinazolin-6-yl)-2-methoxypyridin-3-yl)methanesulfonamide

Tert-butyl 4-(7-chloro-6-(6-methoxy-5-(methylsulfonamido)pyridin-3-yl)quinazolin-4-yl)piperazine-1-carboxylate (120 mg, 0.219 mmol) was dissolved in dichloromethane (3 mL). Under the ice bath condition, trifluoroacetic acid (1 mL) was slowly added to the system. After the addition was completed, the reaction mixture was stirred at room temperature for 1 h. Upon completion of the reaction, under the ice bath condition, the reaction was quenched by adding saturated sodium bicarbonate solution (20 mL), and the reaction mixture was extracted with dichloromethane (30 mL×2). The organic phases were combined, dried over anhydrous sodium sulfate and filtered. The filtrate was concentrated under reduced pressure. N-(5-(7-chloro-4-(piperazin-1-yl)quinazolin-6-yl)-2-methoxypyridin-3-yl)methanesulfonamide was obtained. The product can be used directly in the next reaction without further purification; ESI-MS(m/z): 449.1 [M+H]⁺.

### Step i): Preparation of N-(5-(4-(4-acryloylpiperazin-1-yl)-7-chloroquinazolin-6-yl)-2-methoxypyridin-3-yl)methanesulfonamide

N-(5-(7-chloro-4-(piperazin-1-yl)quinazolin-6-yl)-2-methoxypyridin-3-yl)methanesulfonamide (95 mg, 0.212 mmol) and acrylic acid (15.3 mg, 0.212 mmol) were dissolved in dichloromethane (3 mL). At −78° C., N,N-diisopropylethylamine (137 mg, 1.060 mmol) and HATU (89 mg, 0.233 mmol) were slowly added to the system. After the addition was completed, the reaction mixture was kept at −78° C. and stirred for 0.5 h. Upon completion of the reaction, the reaction was quenched by adding water (10 mL), and the reaction mixture was extracted with dichloromethane (50 mL×2). The organic phases were combined, dried over anhydrous sodium sulfate and filtered. The filtrate was concentrated under reduced pressure. The crude product obtained was purified by Prep-HPLC (Method 2). N-(5-(4-(4-acryloylpiperazin-1-yl)-7-chloroquinazolin-6-yl)-2-methoxypyridin-3-yl)methanesulfonamide was obtained, yield 4.9%; ¹H NMR (400 MHz, DMSO-d₆) δ 9.38 (s, 1H), 8.66 (s, 1H), 8.16 (d, J=2.4 MHz, 1H), 8.02 (s, 2H), 7.83 (d, J=2.4 Hz, 1H), 6.84-6.78 (m, 1H), 6.18-6.13 (m, 1H), 5.74-5.71 (m, 1H), 3.99 (s, 3H), 3.92-3.89 (m, 4H), 3.80 (s, 2H), 3.74 (s, 2H), 3.07 (s, 3H); ESI-MS(m/z): 503.0 [M+H]⁺.

### Example 4

### Preparation of N-(5-(4-(4-acryloylpiperazin-1-yl)quinazolin-6-yl)-2-methoxypyridin-3-yl)methanesulfonamide

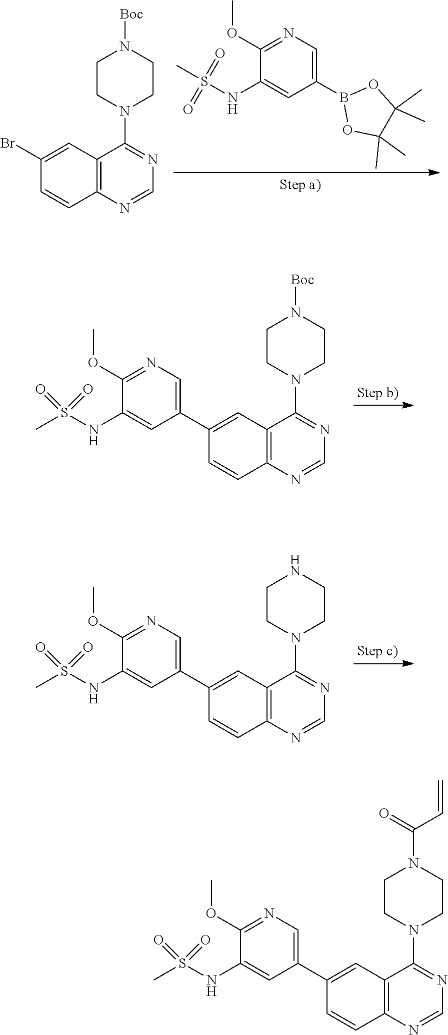

### Step a): Preparation of tert-butyl 4-(6-(6-methoxy-5-(methylsulfonamido)pyridin-3-yl)quinazolin-4-yl)piperazine-1-carboxylate

Tert-butyl 4-(6-bromoquinazolin-4-yl)piperazine-1-carboxylate (300 mg, 0.763 mmol), N-(2-methoxy-5-(4,4,5,5-tetramethyl-1,3,2-dioxaborolan-2-yl)pyridin-3-yl)methanesulfonamide (300 mg, 0.916 mmol), Pd(dppf)Cl₂ (56 mg, 0.076 mmol) and cesium carbonate (751 mg, 2.290 mmol) were added to dioxane/water (10 mL, v/v=4:1). After the addition was completed, in the presense of protective nitrogen, the reaction mixture was kept at 110° C. and stirred for 4 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated under reduced pressure. The crude product obtained was purified by silica gel column chromatography (Eluent: dichloromethane/methanol=50/1 to 20/1). Tert-butyl 4-(6-(6-methoxy-5-(methylsulfonamido)pyridin-3-yl)quinazolin-4-yl)piperazine-1-carboxylate was obtained, yield: 63.7%; ESI-MS(m/z): 515.2 [M+H]⁺.

### Step b): Preparation of N-(2-methoxy-5-(4-(piperazin-1-yl)quinazolin-6-yl)pyridin-3-yl)methanesulfonamide

Tert-butyl 4-(6-(6-methoxy-5-(methylsulfonamido)pyridin-3-yl)quinazolin-4-yl)piperazine-1-carboxylate (250 mg, 0.486 mmol) was dissolved in dichloromethane (8 mL). Under the ice bath condition, TFA (2 mL) was slowly added dropwise. After the addition was completed, the reaction mixture was kept at room temperature and reacted for 1 h. Upon completion of the reaction, the reaction mixture was concentrated under reduced pressure. N-(2-methoxy-5-(4-(piperazin-1-yl)quinazolin-6-yl)pyridin-3-yl)methanesulfonamide was obtained. The product can be used directly in the next reaction without further purification. ESI-MS(m/z): 415.1 [M+H]⁺.

### Step c): Preparation of N-(5-(4-(4-acryloylpiperazin-1-yl)quinazolin-6-yl)-2-methoxypyridin-3-yl)methanesulfonamide

N-(2-methoxy-5-(4-(piperazin-1-yl)quinazolin-6-yl)pyridin-3-yl)methanesulfonamide (200 mg, 0.483 mmol) and HATU (367 mg, 0.966 mmol) were dissolved in dichloromethane (10 mL). At −78° C., N,N-diisopropylethylamine (251 mg, 1.932 mmol) and acrylic acid (52 mg, 0.725 mmol) were slowly added to the system. After the addition was completed, the reaction mixture was kept at −78° C. and stirred for 2 h. Upon completion of the reaction, the reaction was quenched by adding water (50 mL), and the reaction mixture was extracted with dichloromethane (100 mL×2). The organic phases were combined, dried over anhydrous sodium sulfate and filtered. The filtrate was concentrated under reduced pressure. The crude product obtained was purified by Prep-HPLC. N-(5-(4-(4-acryloylpiperazin-1-yl)quinazolin-6-yl)-2-methoxypyridin-3-yl)methanesulfonamide was obtained, yield: 21.8%; ¹H NMR (400 MHz, DMSO-d₆) δ 9.40 (s, 1H), 8.66 (s, 1H), 8.46 (s, 1H), 8.15-8.12 (m, 2H), 8.02 (m, 1H), 7.93-7.91 (m, 1H), 6.87-6.80 (m, 1H), 6.15 (d, J=20.0 Hz, 1H), 5.76 (d, J=12.0 Hz, 1H), 4.00 (s, 3H), 3.89-3.79 (m, 8H), 3.13 (s, 3H); ESI-MS(m/z): 469.0 [M+H]⁺.

### Example 5

### Preparation of N-(5-(4-((S)-4-acryloyl-3-(cyanomethyl)piperazin-1-yl)-2-(((S)-1-methylpyrrolidin-2-yl)methoxy)quinazolin-6-yl)-2-methoxypyridin-3-yl)-2,4-difluorobenzenesulfonamide

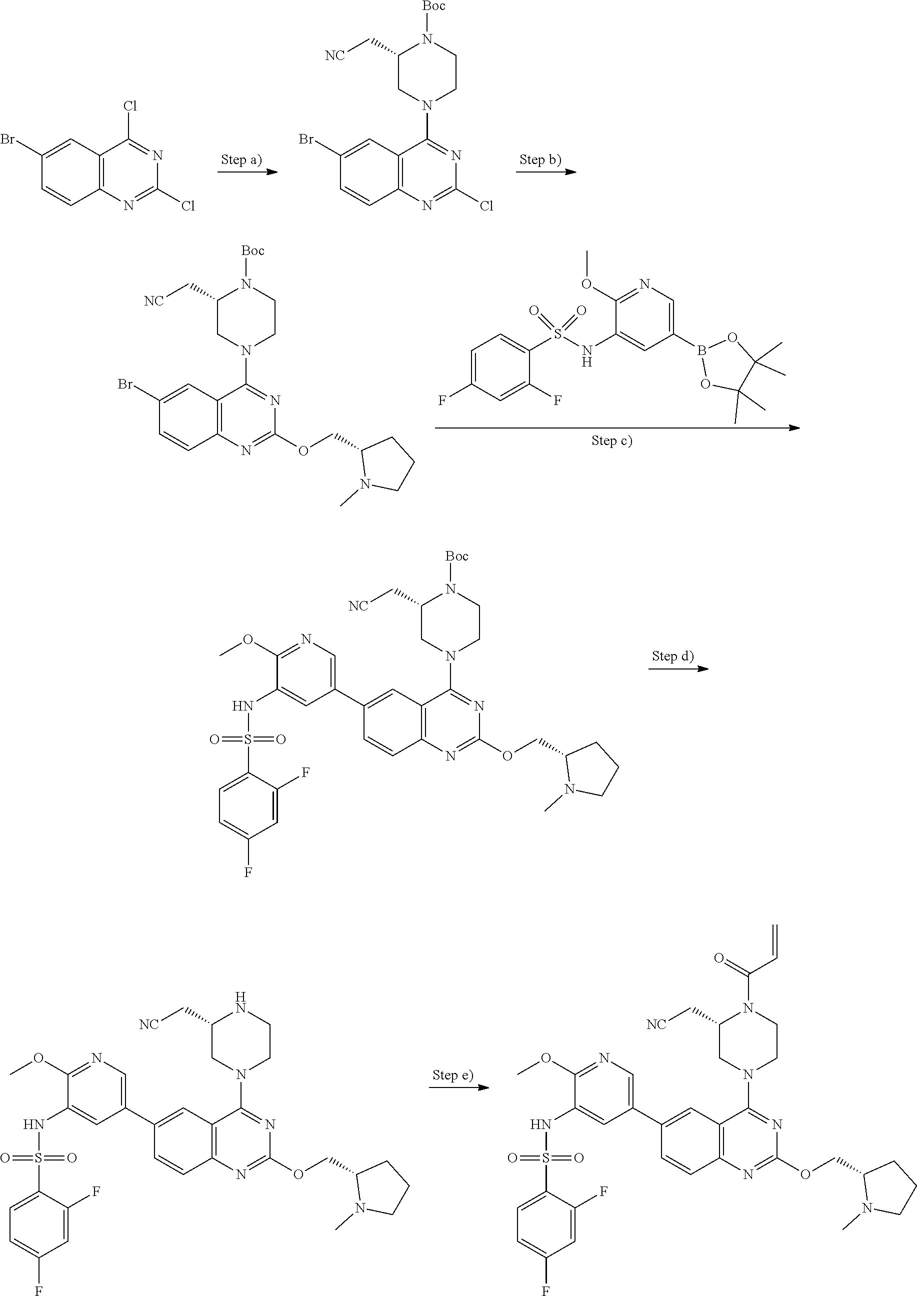

### Step a): Preparation of tert-butyl (S)-4-(6-bromo-2-chloroquinazolin-4-yl)-2-(cyanomethyl)piperazine-1-carboxylate

6-Bromo-2,4-dichloroquinazoline (800 mg, 2.878 mmol), (S)-2-(piperazin-2-yl)acetonitrile (431.65 g, 3.453 mmol) and N,N-diisopropylethylamine (1.87 g, 14.388 mmol) were dissolved in dimethyl sulfoxide (20 mL). After the addition was completed, the reaction mixture was heated to 60° C. and stirred for 1 h, with di-tert-butyl dicarbonate (1.26 g, 5.755 mmol) added. Upon completion of the reaction, the reaction was quenched by adding water (100 mL). The reaction mixture was extracted with ethyl acetate (150 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=5/1 to 3/1). Tert-butyl (S)-4-(6-bromo-2-chloroquinazolin-4-yl)-2-(cyanomethyl)piperazine-1-carboxylate was obtained, yield: 74.4%; ESI-MS(m/z): 466.0[M+H]⁺.

### Step b): Preparation of tert-butyl (S)-4-(6-bromo-2-(((S)-1-methylpyrrolidin-2-yl)methoxy)quinazolin-4-yl)-2-(cyanomethyl)piperazine-1-carboxylate

Sodium hydride (256.96 mg, 6.424 mmol) was suspended in tetrahydrofuran (30 mL) under nitrogen atmosphere. Under the ice bath condition, (S)-(1-methylpyrrolidin-2-yl)methanol (295.50 mg, 2.57 mmol) was added to the system. After the addition was completed, under the ice bath condition, the reaction mixture was stirred for 1 h. Tert-butyl (S)-4-(6-bromo-2-chloroquinazolin-4-yl)-2-(cyanomethyl)piperazine-1-carboxylate (300 mg, 0.642 mmol) was then added to the system. After the addition was completed, the reaction mixture was slowly heated to the room temperature and stirred for 2 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated under reduced pressure. The crude product obtained was purified by silica gel column chromatography (Eluent: dichloromethane/methanol=50/1 to 20/1). Tert-butyl (S)-4-(6-bromo-2-(((S)-1-methylpyrrolidin-2-yl)methoxy)quinazolin-4-yl)-2-(cyanomethyl)piperazine-1-carboxylate was obtained, yield: 85.7%; ESI-MS(m/z): 545.2 [M+H]⁺.

### Step c): Preparation of tert-butyl (S)-2-(cyanomethyl)-4-(6-(5-((2,4-difluorophenyl)sulfonamido)-6-methoxypyridin-3-yl)-2-(((S)-1-methylpyrrolidin-2-yl)methoxy)quinazolin-4-yl)piperazine-1-carboxylate

Tert-butyl (S)-4-(6-bromo-2-(((S)-1-methylpyrrolidin-2-yl)methoxy)quinazolin-4-yl)-2-(cyanomethyl)piperazine-1-carboxylate (300.00 mg, 0.550 mmol), 2,4-difluoro-N-(2-methoxy-5-(4,4,5,5-tetramethyl-1,3,2-dioxaborolan-2-yl)pyridin-3-yl)benzenesulfonamide (351.74 mg, 0.826 mmol), Pd(dppf)Cl₂ (40.29 mg, 0.055 mmol) and cesium carbonate (361.10 mg, 1.101 mmol) were added to dioxane/water (10 mL, v/v=4:1). After the addition was completed, in the presense of protective nitrogen, the reaction mixture was kept at 110° C. and stirred for 4 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated under reduced pressure. The crude product obtained was purified by silica gel column chromatography (Eluent: dichloromethane/methanol=50/1 to 20/1). Tert-butyl (S)-2-(cyanomethyl)-4-(6-(5-((2,4-difluorophenyl)sulfonamido)-6-methoxypyridin-3-yl)-2-(((S)-1-methylpyrrolidin-2-yl)methoxy)quinazolin-4-yl)piperazine-1-carboxylate was obtained, yield: 83.1%; ESI-MS(m/z): 765.3 [M+H]⁺.

### Step d): Preparation of N-(5-(4-((S)-3-(cyanomethyl)piperazin-1-yl)-2-(((S)-1-methylpyrrolidin-2-yl)methoxy)quinazolin-6-yl)-2-methoxypyridin-3-yl)-2,4-difluorobenzenesulfonamide

Tert-butyl (S)-2-(cyanomethyl)-4-(6-(5-((2,4-difluorophenyl)sulfonamido)-6-methoxypyridin-3-yl)-2-(((S)-1-methylpyrrolidin-2-yl)methoxy)quinazolin-4-yl)piperazine-1-carboxylate (350.00 mg, 0.458 mmol) was dissolved in dichloromethane (8 mL). Under the ice bath condition, TFA (2 mL) was slowly added dropwise. After the addition was completed, the reaction mixture was stirred at room temperature for 1 h. Upon completion of the reaction, the reaction mixture was concentrated under reduced pressure. N-(5-(4-((S)-3-(cyanomethyl)piperazin-1-yl)-2-(((S)-1-methylpyrrolidin-2-yl)methoxy)quinazolin-6-yl)-2-methoxypyridin-3-yl)-2,4-difluorobenzenesulfonamide was obtained. The product can be used directly in the next reaction without purification. ESI-MS(m/z): 665.3 [M+H]⁺.

### Step e): Preparation of N-(5-(4-((S)-4-acryloyl-3-(cyanomethyl)piperazin-1-yl)-2-(((S)-1-methylpyrrolidin-2-yl)methoxy)quinazolin-6-yl)-2-methoxypyridin-3-yl)-2,4-difluorobenzenesulfonamide

N-(5-(4-((S)-3-(cyanomethyl)piperazin-1-yl)-2-(((S)-1-methylpyrrolidin-2-yl)methoxy)quinazolin-6-yl)-2-methoxypyridin-3-yl)-2,4-difluorobenzenesulfonamide (250.00 mg, 0.375 mmol) and HATU (285.71 mg, 0.750 mmol) were dissolved in dichloromethane (10 mL). At −78° C. N,N-diisopropylethylamine (244.36 mg, 1.125 mmol) and acrylic acid (40.60 mg, 0.564 mmol) were slowly added to the system. After the addition was completed, the reaction mixture was kept at −78° C. and stirred for 2 h. Upon completion of the reaction, the reaction was quenched by adding water (50 mL). The reaction mixture was extracted with ethyl acetate (100 mL 2). The organic phases were combined, dried over anhydrous sodium sulfate and filtered. The filtrate was concentrated under reduced pressure. The crude product obtained was purified by Prep-HPLC. N-(5-(4-((S)-4-acryloyl-3-(cyanomethyl)piperazin-1-yl)-2-(((S)-1-methylpyrrolidin-2-yl)methoxy)quinazolin-6-yl)-2-methoxypyridin-3-yl)-2,4-difluorobenzenesulfonamide was obtained, yield: 10.4%; ¹H NMR (400 MHz, DMSO-d₆) δ 10.52-9.59 (m, 1H), 8.45 (s, 1H), 8.12 (s, 1H), 7.96-7.91 (m, 2H), 7.78-7.70 (m, 2H), 7.52-7.48 (m, 1H), 7.23-7.18 (m, 1H), 6.98-6.70 (m, 1H), 6.22 (d, J=8.0 Hz, 1H), 5.73-5.71 (m, 1H), 5.08-4.71 (m, 1H), 4.44-4.03 (m, 5H), 3.75-3.71 (m, 1H), 3.69 (s, 3H), 3.53-3.35 (m, 4H), 3.10-2.91 (m, 4H), 2.70 (s, 1H), 2.32-2.40 (m, 1H), 2.10-1.98 (m, 1H), 1.77-1.72 (m, 3H); ESI-MS(m/z): 719.5 [M+H]⁺.

### Example 6

### Preparation of (E)-2,4-difluoro-N-(2-methoxy-5-(4-(4-(4-oxopent-2-enoyl)piperazin-1-yl)quinazoline-6-yl)pyridin-3-yl)benzenesulfonamide

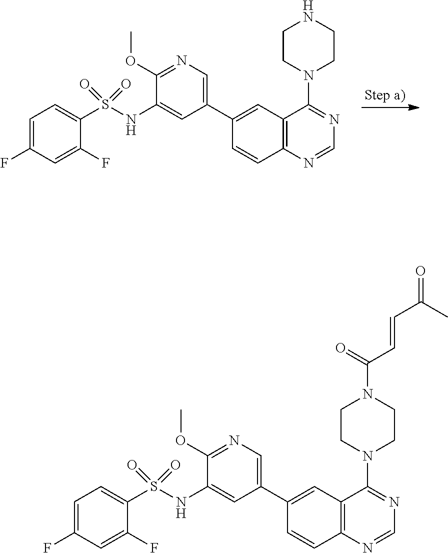 [SMILES: COc1ncc(-c2ccc3ncnc(N4CCNCC4)c3c2)cc1NS(=O)(=O)c1ccc(F)cc1F ; COc1ncc(-c2ccc3ncnc(N4CCN(C(=O)/C=C/C(C)=O)CC4)c3c2)cc1NS(=O)(=O)c1ccc(F)cc1F]

2,4-difluoro-N-(2-methoxy-5-(4-(piperazin-1-yl)quinazolin-6-yl)pyridin-3-yl)benzenesulfonamide (40 mg, 0.078 mmol) and (E)-4-oxopent-2-enoic acid (9 mg, 0.079 mmol) were dissolved in tetrahydrofuran (2 mL). At −78° C., N,N-diisopropylethylamine (101 mg, 0.781 mmol) and 1-propylphosphonic anhydride (75 mg, 0.236 mmol, 50% wt) were slowly added to the system. After the addition was completed, the reaction mixture was kept at −78° C. and stirred for 1 h. Upon completion of the reaction, the reaction was quenched by adding water (10 mL), and the reaction mixture was extracted with dichloromethane (30 mL×2). The organic phases were combined, dried over anhydrous sodium sulfate and filtered. The filtrate was concentrated under reduced pressure to provide the crude residue. The crude product obtained was purified by Prep-HPLC. (E)-2,4-difluoro-N-(2-methoxy-5-(4-(4-(4-oxopent-2-enoyl)piperazin-1-yl)quinazoline-6-yl)pyridin-3-yl)benzenesulfonamide was obtained, yield: 38.6%; ¹H NMR (400 MHz, DMSO-d₆) δ 10.34 (s, 1H), 8.67 (s, 1H), 8.46 (s, 1H), 8.14-8.08 (m, 2H), 8.03-7.99 (m, 1H), 7.95-7.90 (m, 1H), 7.81-7.74 (m, 1H), 7.61-7.52 (m, 1H), 7.45 (d, J=16.0 Hz, 1H), 7.25-7.18 (m, 1H), 6.74 (d, J=16.0 Hz, 1H), 3.93-3.87 (m, 6H), 3.83-3.79 (m, 2H), 3.68 (s, 3H), 2.37 (s, 3H); ESI-MS(m/z): 609.0 [M+H]⁺.

### Example 7

### Preparation of (S)—N-(5-(4-(4-acryloyl-2-methylpiperazin-1-yl)quinazolin-6-yl)-2-methoxypyridin-3-yl)-2,4-difluorobenzenesulfonamide

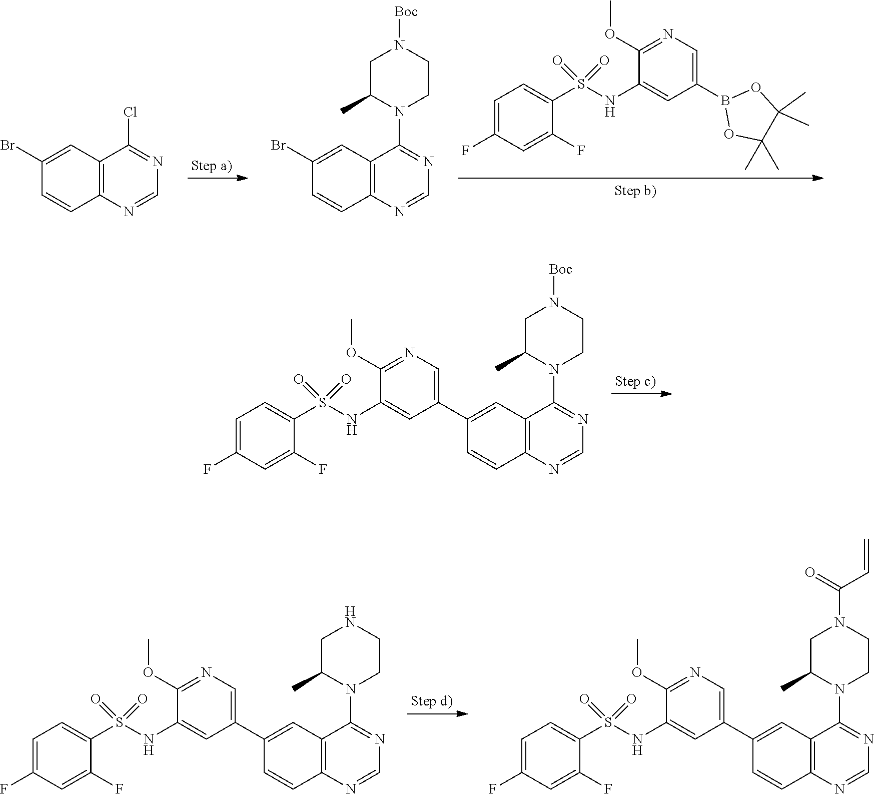

### Step a): Preparation of tert-butyl (S)-(6-bromoquinazolin-4-yl)-3-methylpiperazine-1-carboxylate

6-Bromo-4-chloroquinazoline (1.0 g, 4.133 mmol) and tert-butyl (S)-3-methylpiperazine-1-carboxylate (827.0 g, 4.133 mmol) were dissolved in dimethyl sulfoxide (10 mL), with N,N-diisopropylethylamine (1.1 g, 8.266 mmol) added. After the addition was completed, the reaction mixture was heated to 50° C. and stirred for 1 h. Upon completion of the reaction, the reaction was quenched by adding water (50 mL). The reaction mixture was extracted with ethyl acetate (100 mL×2 times). The organic phases were combined, dried over anhydrous sodium sulfate and filtered. The filtrate was concentrated under reduced pressure. The crude product obtained was purified by silica gel column chromatography (Eluent: petroleum ether/ethyl acetate=10/1 to 3/1). (S)-4-(6-bromoquinazolin-4-yl)-3-methylpiperazine-1-carboxylate was obtained, yield: 51.8%; ESI-MS(m/z): 407.10 [M+H]⁺.

### Step b): Preparation of tert-butyl (S)-4-(6-(5-((2,4-difluorophenyl)sulfonamido)-6-methoxypyridin-3-yl)quinazolin-4-yl)-3-methylpiperazine-1-carboxylate

(S)-4-(6-bromoquinazolin-4-yl)-3-methylpiperazine-1-carboxylate (377 mg, 0.929 mmol), 2,4-difluoro-N-(2-methoxy-5-(4,4,5,5-tetramethyl-1,3,2-dioxaborolan-2-yl)pyridin-3-yl)benzenesulfonamide (330 mg, 0.774 mmol), Pd(dppf)Cl₂ (113 mg, 0.155 mmol) and cesium carbonate (503 mg, 1.548 mmol) were added to dioxane/water mixed solvent (10 mL, v/v=10:1). After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (100 mL×2 times). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=2/1 to 1/1). Tert-butyl (S)-4-(6-(5-((2,4-difluorophenyl)sulfonamido)-6-methoxypyridin-3-yl)quinazolin-4-yl)-3-methylpiperazine-1-carboxylate was obtained, yield: 54.8%; ESI-MS(m/z): 613.20 [M+H]⁺.

### Step c): Preparation of (S)-2,4-difluoro-N-(2-methoxy-5-(4-(2-methylpiperazin-1-yl)quinazolin-6-yl)pyridin-3-yl)benzenesulfonamide

Tert-butyl (S)-4-(6-(5-((2,4-difluorophenyl)sulfonamido)-6-methoxypyridin-3-yl)quinazolin-4-yl)-3-methylpiperazine-1-carboxylate (260.0 mg, 0.415 mmol) was dissolved in dichloromethane (4 mL). Under the ice bath condition, trifluoroacetic acid (4 mL) was slowly added dropwise. After the addition was completed, the reaction mixture was stirred at room temperature for 1 h. Upon completion of the reaction, the reaction mixture was concentrated under reduced pressure. (S)-2,4-difluoro-N-(2-methoxy-5-(4-(2-methylpiperazin-1-yl)quinazolin-6-yl)pyridin-3-yl)benzenesulfonamide was obtained. ESI-MS(m/z): 527.2 [M+H]⁺.

### Step d): Preparation of (S)—N-(5-(4-(4-acryloyl-2-methylpiperazin-1-yl)quinazolin-6-yl)-2-methoxypyridin-3-yl)-2,4-difluorobenzenesulfonamide

(S)-2,4-difluoro-N-(2-methoxy-5-(4-(2-methylpiperazin-1-yl)quinazolin-6-yl)pyridin-3-yl)benzenesulfonamide (130.0 mg, 0.247 mmol) was dissolved in dichloromethane (10 mL). The reaction system was cooled to −78° C. with N,N-diisopropylethylamine (127.5 mg, 0.988 mmol), acrylic acid (17.8 mg, 0.247 mmol) and HATU (112.7 mg, 0.296 mmol) added successively. After the addition was completed, the reaction mixture was kept at −78° C. and stirred for 1 h. Upon completion of the reaction, the reaction was quenched by adding water (5 mL). The reaction mixture was extracted by adding dichloromethane (10 mL/2 times). The organic phases were combined, dried over anhydrous sodium sulfate and filtered. The filtrate was concentrated under reduced pressure. The crude product obtained was purified by Prep-HPLC. (S)—N-(5-(4-(4-acryloyl-2-methylpiperazin-1-yl)quinazolin-6-yl)-2-methoxypyridin-3-yl)-2,4-difluorobenzenesulfonamide was obtained, yield: 10.5%; ¹H NMR (400 MHz, DMSO-d₆) δ 10.5 (s, 1H), 8.74 (s, 1H), 8.53 (s, 1H), 8.19-7.87 (m, 4H), 7.85 (s, 1H), 7.65 (s, 1H), 7.30 (s, 1H), 7.0 (s, 1H), 6.30-6.28 (m, 1H), 5.85-5.77 (m, 1H), 4.84 (s, 1H), 4.53 (d, J=12.0 Hz, 1H), 4.44-4.21 (m, 2H), 4.05 (d, J=16.0 Hz, 1H), 3.78-3.70 (m, 4H), 3.25-3.03 (m, 1H), 1.0 (t, J=12.0 Hz, 3H); ESI-MS(m/z): 581.2 [M+H]⁺.

### Example 8

### Preparation of (S,E)-2,4-difluoro-N-(2-methoxy-5-(4-(2-methyl-4-(4-oxopent-2-enoyl)piperazin-1-yl)quinazolin-6-yl)pyridin-3-yl)benzenesulfonamide

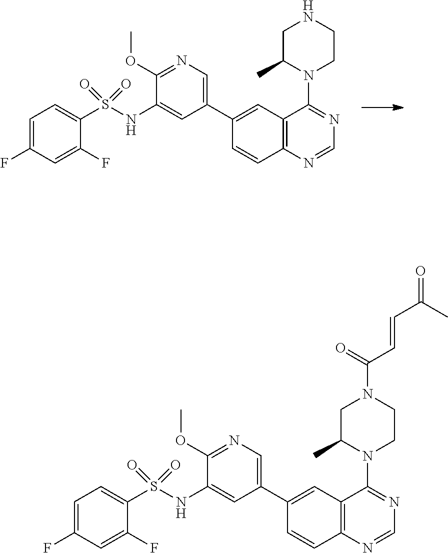 [SMILES: COc1ncc(-c2ccc3ncnc(N4CCNC[C@@H]4C)c3c2)cc1NS(=O)(=O)c1ccc(F)cc1F ; COc1ncc(-c2ccc3ncnc(N4CCN(C(=O)/C=C/C(C)=O)C[C@@H]4C)c3c2)cc1NS(=O)(=O)c1ccc(F)cc1F]

(S)-2,4-difluoro-N-(2-methoxy-5-(4-(2-methylpiperazin-1-yl)quinazolin-6-yl)pyridin-3-yl)benzenesulfonamide (60.0 mg, 0.114 mmol) was dissolved in tetrahydrofuran (2 mL). The reaction system was cooled to −78° C., with N,N-diisopropylethylamine (73.53 mg, 0.570 mmol), 3-acetylacrylic acid (13.0 mg, 0.114 mmol) and 50% T₃P ethyl acetate solution (72.5 mg, 0.114 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC. (S,E)-2,4-difluoro-N-(2-methoxy-5-(4-(2-methyl-4-(4-oxopent-2-enoyl)piperazin-1-yl)quinazolin-6-yl)pyridin-3-yl)benzenesulfonamide was obtained, yield: 28.2%. ¹H NMR (400 MHz, DMSO-d₆) δ 10.5 (s, 1H), 8.67 (s, 1H), 8.46 (s, 1H), 8.11-8.01 (m, 3H), 7.92 (d, J=8.0 Hz, 1H), 7.79-7.77 (m, 1H), 7.57-7.48 (m, 2H), 7.22-7.21 (m, 1H), 6.79-6.70 (m, 1H), 4.81-4.76 (m, 1H), 4.43 (d, J=12.0 Hz, 1H), 4.30-4.20 (m, 2H), 4.01 (d, J=12.0 Hz, 1H), 3.70-3.67 (m, 5H), 2.38-2.21 (m, 3H), 1.43-1.31 (m, 3H); ESI-MS(m/z): 622.6 [M+H]⁺.

### Example 9

### Preparation of (S)-2,4-difluoro-N-(5-(4-(4-(2-fluoroacryloyl)-2-methylpiperazin-1-yl)quinazolin-6-yl)-2-methoxypyridin-3-yl)benzenesulfonamide

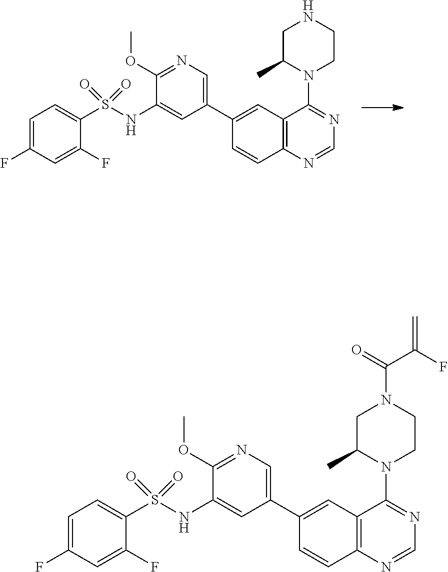 [SMILES: COc1ncc(-c2ccc3ncnc(N4CCNC[C@@H]4C)c3c2)cc1NS(=O)(=O)c1ccc(F)cc1F ; C=C(F)C(=O)N1CCN(c2ncnc3ccc(-c4cnc(OC)c(NS(=O)(=O)c5ccc(F)cc5F)c4)cc23)[C@@H](C)C1]

(S)-2,4-difluoro-N-(2-methoxy-5-(4-(2-methylpiperazin-1-yl)quinazolin-6-yl)pyridin-3-yl)benzenesulfonamide (75.0 mg, 0.143 mmol) was dissolved in tetrahydrofuran (2 mL). The reaction system was cooled to −78° C., with N,N-diisopropylethylamine (91.9 mg, 0.713 mmol), 2-fluoroacrylic acid (12.9 mg, 0.143 mmol) and 50% T₃P ethyl acetate solution (91.0 mg, 0.143 mmol) added successively After the addition was completed, the reaction mixture was kept at −78° C. and stirred for 1 h. Upon completion of the reaction, the reaction solution was diluted by adding acetonitrile (0 mL) and purified by Prep-HPLC. (S)-2,4-difluoro-N-(5-(4-(4-(2-fluoroacryloyl)-2-methylpiperazin-1-yl)quinazolin-6-yl)-2-methoxypyridin-3-yl)benzenesulfonamide was obtained, yield: 25.8%; ¹H NMR (400 MHz, DMSO-d₆) δ 10.3 (brs, 1H), 8.64 (s, 1H), 8.44 (s, 1H), 8.08-8.00 (m, 3H), 7.99-7.88 (m, 1H), 7.88-7.73 (m, 1H), 7.54-7.52 (m, 1H), 7.19 (d, J=4.0 Hz, 1H), 5.34-5.18 (m, 2H), 4.80 (s, 1H), 4.40-3.85 (m, 3H), 3.83-3.49 (m, 6H), 1.30 (d, J=8.0 Hz, 3H); ESI-MS(m/z): 599.6 [M+H]⁺.

### Example 10

### Preparation of (E)-N-(5-(4-(4-(3-(1H-1,2,3-triazol-1-yl)acryloyl)piperazin-1-yl)quinazolin-6-yl)-2-methoxypyridin-3-yl)-2,4-difluorobenzenesulfonamide

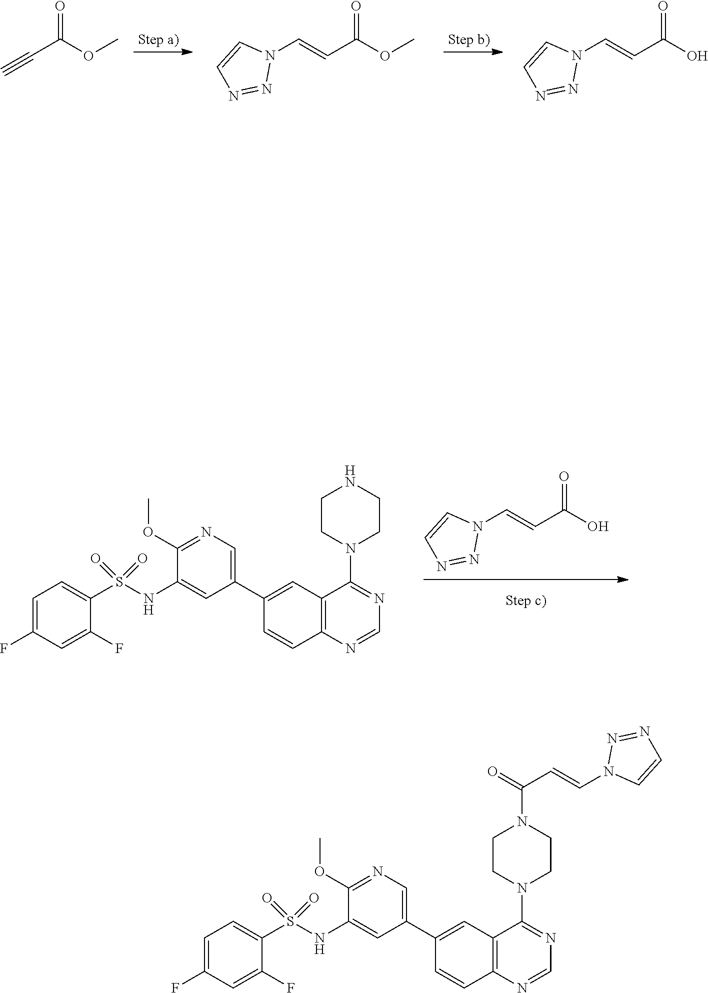

### Step a): Preparation of methyl (E)-3-(1H-1,2,3-triazol-1-yl)acrylate

Triazole (5 g, 72.359 mmol) was dissolved in methyl propiolate (8 mL), the reaction solution was heated to 100° C. and stirred for 12 h. Upon completion of the reaction, add ethyl acetate to the reaction solution (50 mL). After yellow solid precipitation appeared, the solution was filtered. The filter cake was collected and eluted with appropriate amount of ethyl acetate, vacuum dried to obtain methyl (E)-3-(1H-1,2,3-triazol-1-yl)acrylate, yield: 23.5%; ESI-MS(m/z): 154.1 [M+H]⁺.

### Step b): Preparation of (E)-3-(1H-1,2,3-triazol-1-yl)acrylic acid

Methyl (E)-3-(1H-1,2,3-triazol-1-yl)acrylate (500 mg, 3.266 mmol) was dissolved in dilute H₂SO₄ (1 M, 10 mL). The reaction solution was heated to reflux, stirred and reacted for 3 h. Upon completion of the reaction, the solution was filtered. The filter cake was collected and washed with appropriate amount of water, vacuum dried to obtain (E)-3-(1H-1,2,3-triazol-1-yl)acrylic acid, yield: 66.1%; ESI-MS(m/z): 138.0 [M+H]⁺.

### Step c): Preparation of (E)-N-(5-(4-(4-(3-(1H-1,2,3-triazol-1-yl)acryloyl)piperazin-1-yl)quinazolin-6-yl)-2-methoxypyridin-3-yl)-2,4-difluorobenzenesulfonamide

(E)-3-(1H-1,2,3-triazol-1-yl)acrylic acid (50 mg, 0.359 mmol) and HATU (71 mg, 0.187 mmol) were dissolved in N,N-dimethylformamide (2 mL). The reaction system was cooled to −41° C., with N,N-diisopropylethylamine (101 mg, 0.781 mmol) and 2,4-difluoro-N-(2-methoxy-5-(4-(piperazin-1-yl)quinazolin-6-yl)pyridin-3-yl)benzenesulfonamide (80 mg, 0.156 mmol) added successively. After the addition was completed, the reaction mixture was kept at −41° C. and stirred for 1 h. Upon completion of the reaction, the reaction solution was purified by Prep-HPLC. (E)-N-(5-(4-(4-(3-(1H-1,2,3-triazol-1-yl)acryloyl)piperazin-1-yl)quinazolin-6-yl)-2-methoxypyridin-3-yl)-2,4-difluorobenzenesulfonamide was obtained, yield: 43.3%; ¹H NMR (400 MHz, DMSO-d₆) δ 10.33 (s, 1H), 8.75 (s, 1H), 8.67 (s, 1H), 8.49-8.48 (m, 1H), 8.25-8.21 (m, 1H), 8.16-8.09 (m, 2H), 8.03 (d, J=8.0 Hz, 1H), 7.94-7.91 (m, 2H), 7.81-7.75 (m, 1H), 7.60-7.54 (m, 1H), 7.49-7.44 (m, 1H), 7.25-7.19 (m, 1H), 3.97-3.93 (m, 6H), 3.87-3.84 (m, 2H), 3.68 (s, 3H); ESI-MS(m/z): 634.0 [M+H]⁺.

### Example 11

### Preparation of (E)-N-(5-(4-(4-(4-(dimethylamino)but-2-enoyl)piperazin-1-yl)quinazolin-6-yl)-2-methoxypyridin-3-yl)-2,4-difluorobenzenesulfonamide

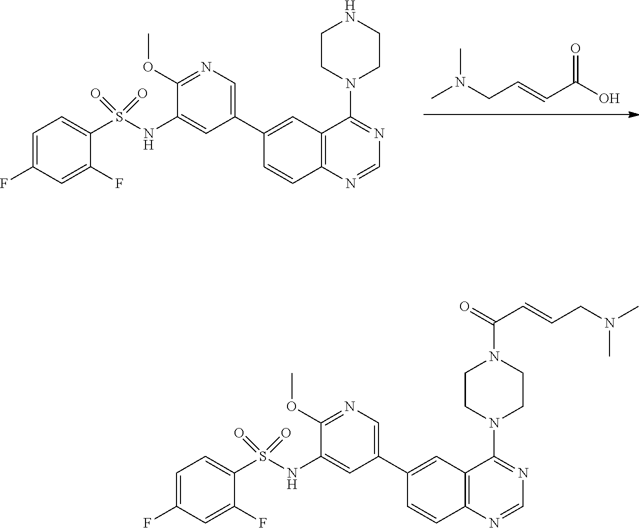

(E)-4-(dimethylamino)but-2-enoic acid (40 mg 0.310 mmol) and HATU (71 mg, 0.187 mmol) were dissolved in N,N-dimethylformamide (2 mL). The reaction system was cooled to 0° C., with N,N-diisopropylethylamine (101 mg, 0.781 mmol) and 2,4-difluoro-N-(2-methoxy-5-(4-(piperazin-1-yl)quinazolin-6-yl)pyridin-3-yl)benzenesulfonamide (80 mg, 0.156 mmol) added successively. After the addition was completed, the reaction mixture was kept at 0° C. and stirred for 1 h. Upon completion of the reaction, the reaction solution was purified by Prep-HPLC. (E)-N-(5-(4-(4-(4-(dimethylamino)but-2-enoyl)piperazin-1-yl)quinazolin-6-yl)-2-methoxypyridin-3-yl)-2,4-difluorobenzenesulfonamide was obtained, yield: 47.6%; ¹H NMR (400 MHz, DMSO-d₆) δ 8.65 (s, 1H), 8.38-8.36 (m, 1H), 8.13-8.02 (m, 3H), 7.96 (d, J=8.0 Hz, 1H), 7.92-7.88 (m, 1H), 7.82-7.75 (m, 1H), 7.54-7.48 (m, 1H), 7.22-7.16 (m, 1H), 6.68-6.65 (m, 2H), 3.88-3.85 (m, 4H), 3.84-3.81 (m, 2H), 3.79-3.76 (m, 2H), 3.70 (s, 3H), 3.22-3.19 (m, 2H), 2.28 (s, 6H); ESI-MS(m/z): 624.0 [M+H]⁺.

### Example 12

### Preparation of N-(5-(4-(2-acryloyl-2,6-diazaspiro[3.4]octan-6-yl)quinazolin-6-yl)-2-methoxypyridin-3-yl)-2,4-difluorobenzenesulfonamide

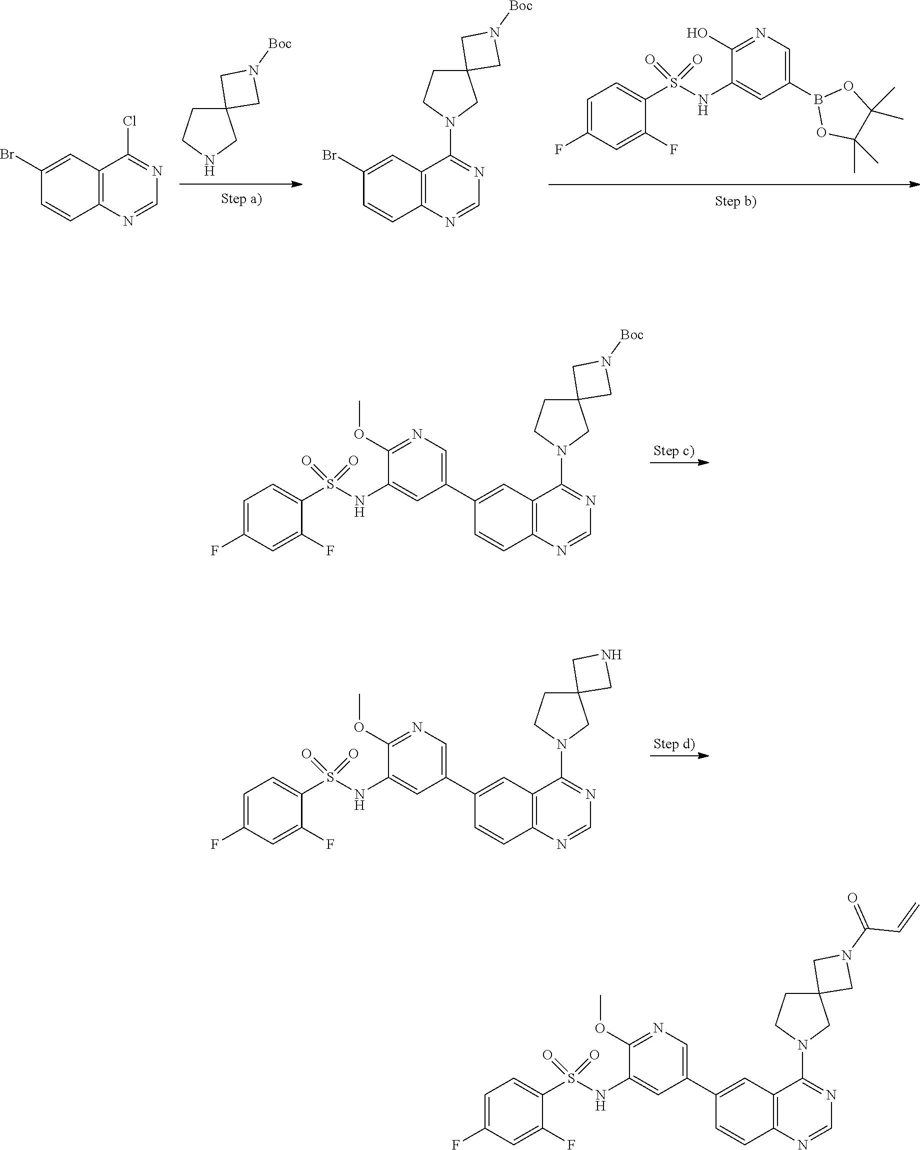

### Step a): Preparation of tert-butyl 6-(6-bromoquinazolin-4-yl)-2,6-diazaspiro[3.4]octane-2-carboxylate

6-Bromo-4-chloroquinazoline (500 mg, 2.06 mmol) and tert-butyl 2,6-diazaspiro[3.4]octane-2-carboxylate (523 mg, 2.46 mmol) were dissolved in dimethyl sulfoxide (10 mL), with N,N-diisopropylethylamine (799 mg, 6.18 mmol) added. After the addition was completed, the reaction mixture was heated to 50° C. and stirred for 1 h. Upon completion of the reaction, the reaction solution was cooled to 25° C. and added to methyl tert-butyl ether (120 mL) dropwise. The mixture was stirred for 20 min. After solid precipitation appeared, the solution was filtered. The solid was vacuum dried to obtain tert-butyl 6-(6-bromoquinazolin-4-yl)-2,6-diazaspiro[3.4]octane-2-carboxylate, yield: 46.4%; ESI-MS(m/z): 419.1[M+H]⁺.

### Step b): Preparation of tert-butyl 6-(6-(5-((2,4-difluorophenyl)sulfonamido)-6-methoxypyridin-3-yl)quinazolin-4-yl)-2,6-diazaspiro[3.4]octane-2-carboxylate

Tert-butyl 6-(6-bromoquinazolin-4-yl)-2,6-diazaspiro[3.4]octane-2-carboxylate (350 mg, 0.835 mmol), 2,4-difluoro-N-(2-hydroxy-5-(4,4,5,5-tetramethyl-1,3,2-dioxaborolan-2-yl)pyridin-3-yl)benzenesulfonamide (350 mg, 0.835 mmol), Pd(dppf)Cl₂ (120 mg, 0.167 mmol) and cesium carbonate (540 mg, 1.67 mmol) were added to dioxane/water mixed solvent (16.5 mL, v/v=10:1). After the addition was completed, in the presense of protective nitrogen, the reaction mixture was heated to 90° C. and stirred for 0.5 h. Upon completion of the reaction, the reaction mixture was cooled to 25° C. with reaction quenched by adding water (20 mL), extracted with ethyl acetate (20 mL×3). The organic phase was washed with saturated brine (15 mL), and concentrated under reduced pressure. The crude product obtained was purified by silica gel column chromatography (Eluent: methanol/dichloromethane=100/1 to 96/4). Tert-butyl 6-(6-(5-((2,4-difluorophenyl)sulfonamido)-6-methoxypyridin-3-yl)quinazolin-4-yl)-2,6-diazaspiro[3.4]octane-2-carboxylate (280 mg) was obtained, yield: 52.4%; ESI-MS(m/z): 639.21 [M+H]⁺.

### Step c): Preparation of N-(5-(4-(2,6-diazaspiro[3.4]octan-6-yl)quinazolin-6-yl)-2-methoxypyridin-3-yl)-2,4-difluorobenzenesulfonamide

Tert-butyl 6-(6-(5-((2,4-difluorophenyl)sulfonamido)-6-methoxypyridin-3-yl)quinazolin-4-yl)-2,6-diazaspiro[3.4]octane-2-carboxylate (280 mg, 0.5 mmol) was dissolved in dichloromethane (10 mL). Under the ice bath condition, trifluoroacetic acid (2 mL) was slowly added dropwise. After the addition was completed, the reaction mixture was stirred at room temperature for 1 h. Upon completion of the reaction, the reaction mixture was concentrated under reduced pressure. The crude product obtained was added to methyl tert-butyl ether and slurried for 20 min to precipitate solid and filtered. N-(5-(4-(2,6-diazaspiro[3.4]octan-6-yl)quinazolin-6-yl)-2-methoxypyridin-3-yl)-2,4-difluorobenzenesulfonamide trifluoroacetate was obtained. ESI-MS(m/z): 539.16 [M+H]⁺.

### Step d): Preparation of N-(5-(4-(2-acryloyl-2,6-diazaspiro[3.4]octan-6-yl)quinazolin-6-yl)-2-methoxypyridin-3-yl)-2,4-difluorobenzenesulfonamide

N-(5-(4-(2,6-diazaspiro[3.4]octan-6-yl)quinazolin-6-yl)-2-methoxypyridin-3-yl)-2,4-difluorobenzenesulfonamide trifluoroacetate (100 mg, 0.153 mmol) was added to tetrahydrofuran (2 mL). In the presense of protective nitrogen, the reaction system was cooled to −70° C., with N,N-diisopropylethylamine (118 mg, 0.918 mmol), acrylic acid (11 mg, 0.153 mmol) and T₃P (195 mg, 0.307 mmol) added successively. After the addition was completed, the reaction mixture was kept at −70° C. and stirred for 0.5 h. Upon completion of the reaction, the product was purified by Prep-HPLC. N-(5-(4-(2-acryloyl-2,6-diazaspiro[3.4]octan-6-yl)quinazolin-6-yl)-2-methoxypyridin-3-yl)-2,4-difluorobenzenesulfonamide (20 mg) was obtained, yield: 22.0%; ¹H NMR (400 MHz, DMSO-d₆) δ 10.33 (brs, 1H), 8.50-8.47 (s, 2H), 8.46 (s, 1H), 8.03-8.00 (m, 2H), 7.82-7.75 (m, 2H), 7.59-7.54 (m, 1H), 7.24-7.19 (m, 1H), 6.35-6.28 (m, 1H), 6.13 (d, J=16.0 Hz, 1H), 5.69 (d, J=12.0 Hz, 1H), 4.32 (d, J=8.0 Hz, 1H), 4.21-4.19 (m, 3H), 4.05-4.00 (m, 3H), 3.94 (d, J=12 Hz, 1H), 3.69 (s, 3H), 2.27-2.23 (m, 2H); ESI-MS(m/z): 593.0 [M+H]⁺.

### Example 13

### Preparation of (E)-2,4-difluoro-N-(2-methoxy-5-(4-(2-(4-oxopent-2-enoyl)-2,6-diazaspiro[3.4]octan-4-yl)quinazolin-6-yl)pyridin-3-yl)benzenesulfonamide

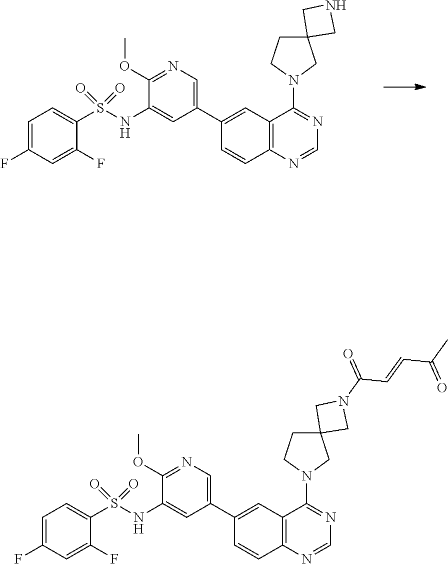 [SMILES: COc1ncc(-c2ccc3ncnc(N4CCC5(CNC5)C4)c3c2)cc1NS(=O)(=O)c1ccc(F)cc1F ; COc1ncc(-c2ccc3ncnc(N4CCC5(CN(C(=O)/C=C/C(C)=O)C5)C4)c3c2)cc1NS(=O)(=O)c1ccc(F)cc1F]

N-(5-(4-(2,6-diazaspiro[3.4]octan-6-yl)quinazolin-6-yl)-2-methoxypyridin-3-yl)-2,4-difluorobenzenesulfonamide trifluoroacetate (100.0 mg, 0.154 mmol) was added to tetrahydrofuran (2 mL). The reaction system was cooled to −70° C., with N,N-diisopropylethylamine (121 mg, 0.924 mmol), acrylic acid (18 mg, 0.154 mmol) and T₃P (170 mg, 0.278 mmol) added successively. After the addition was completed, the reaction mixture was kept at −70° C. and stirred for 0.5 h. Upon completion of the reaction, the reaction mixture was filtered and purified by Prep-HPLC. (E)-2,4-difluoro-N-(2-methoxy-5-(4-(2-(4-oxopent-2-enoyl)-2,6-diazaspiro[3.4]octan-6-yl)quinazolin-6-yl)pyridin-3-yl)benzenesulfonamide was obtained, yield: 34.0%; ¹H NMR (400 MHz, DMSO-d₆) δ 10.32 (br, 1H), 8.49 (s, 1H), 8.45 (s, 1H), 8.37 (s, 1H), 8.03-7.99 (m, 2H), 7.82 (d, J=8.0 Hz, 1H), 7.81-7.75 (m, 1H), 7.59-7.53 (m, 1H), 7.23-7.19 (m, 1H), 6.90 (d, J=16.0 Hz, 1H), 6.74 (d, J=12.0 Hz, 1H), 4.42 (d, J=8.0 Hz, 1H), 4.31 (m, J=8.0 Hz, 1H), 4.30-4.20 (m, 2H), 4.15-4.05 (m, 1H), 4.03-3.97 (m, 3H), 3.69 (s, 3H), 2.34 (s, 3H), 2.28-2.25 (m, 2H); ESI-MS(m/z): 635.0 [M+H]⁺.

### Example 14

### Preparation of N-(5-(4-((2S,5R)-4-acryloyl-2,5-dimethylpiperazin-1-yl)quinazolin-6-yl)-2-methoxypyridin-3-yl)-2,4-difluorobenzenesulfonamide

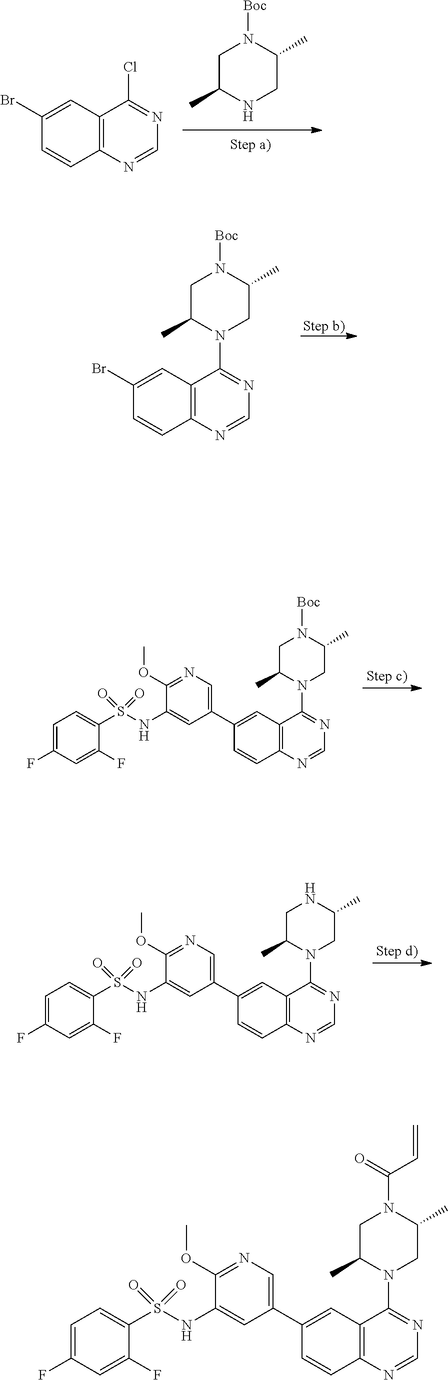

### Step a): Preparation of tert-butyl (2R,5S)-4-(6-bromoquinazolin-4-yl)-2,5-dimethylpiperazine-1-carboxylate

6-Bromo-4-chloroquinazoline (300 mg, 1.23 mmol) and tert-butyl (2R,5S)-2,5-dimethylpiperazine-1-carboxylate (396 mg, 1.84 mmol) were dissolved in dimethyl sulfoxide (5.0 mL), with N,N-diisopropylethylamine (476 mg, 3.69 mmol) added. After the addition was completed, the reaction mixture was heated to 50° C. and stirred for 0.5 h. Upon completion of the reaction, the reaction mixture was cooled to 250° C., with reaction quenched by adding water (20 mL), extracted with ethyl acetate (20 mL×2). The organic phases were combined, washed with saturated brine (15 mL×3), concentrated under reduced pressure. The crude product obtained was purified by silica gel column chromatography (Eluent: petroleum ether/ethyl acetate=10/1 to 3/1). Tert-butyl (2R,5S)-4-(6-bromoquinazolin-4-yl)-2,5-dimethylpiperazine-1-carboxylate was obtained, yield: 96.3%; ESI-MS(m/z): 421.1 [M+H]⁺.

### Step b): Preparation of tert-butyl (2R,5S)-4-(6-(5-((2,4-difluorophenyl)sulfonamido)-6-methoxypyridin-3-yl)quinazolin-4-yl)-2,5-dimethylpiperazine-1-carboxylate

Tert-butyl (2R,5S)-4-(6-bromoquinazolin-4-yl)-2,5-dimethylpiperazine-1-carboxylate (450 mg, 1.068 mmol), 2,4-difluoro-N-(2-hydroxy-5-(4,4,5,5-tetramethyl-1,3,2-dioxaborolan-2-yl)pyridin-3-yl)benzenesulfonamide (450 mg, 1.068 mmol), Pd(dppf)Cl₂ (153 mg, 0.209 mmol) and cesium carbonate (693 mg, 2.132 mmol) were added to dioxane/water mixed solvent (22 mL, v/v=10:1). After the addition was completed, in the presense of protective nitrogen, the reaction mixture was heated to 100° C. and stirred for 0.5 h. Upon completion of the reaction, the reaction mixture was cooled to 250° C. The reaction was quenched by adding water (60 mL). The reaction mixture was extracted with ethyl acetate (40 mL×2 times). The organic phases were combined, washed with saturated brine (15 mL×1), dried over anhydrous sodium sulfate and filtered. The filtrate was concentrated under reduced pressure. The crude product obtained was purified by silica gel column chromatography (Eluent: petroleum ether/ethyl acetate=20/1 to 1/1). Tert-butyl (2R,5S)-4-(6-(5-((2,4-difluorophenyl)sulfonamido)-6-methoxypyridin-3-yl)quinazolin-4-yl)-2,5-dimethylpiperazine-1-carboxylate was obtained, yield: 70.1%; ESI-MS(m/z): 641.2 [M+H]⁺.

### Step c): Preparation of N-(5-(4-((2S,5R)-2,5-dimethylpiperazin-1-yl)quinazolin-6-yl)-2-methoxypyridin-3-yl)-2,4-difluorobenzenesulfonamide trifluoroacetate

Tert-buty (2R,5S)-4-(6-(5-((2,4-difluorophenyl)sulfonamido)-6-methoxypyridin-3-yl)quinazolin-4-yl)-2,5-dimethylpiperazine-1-carboxylate (400 mg, 0.624 mmol) was dissolved in dichloromethane (10 mL). Under the ice bath condition, trifluoroacetic acid (2 mL) was slowly added dropwise. After the addition was completed, the reaction mixture was stirred at room temperature for 1 h. Upon completion of the reaction, the reaction mixture was concentrated under reduced pressure. The oily crude product was obtained, added to methyl tert-butyl ether (20 mL) and slurried for 10 min. The solution was filtered after white solid precipitation appeared. N-(5-(4-((2S,5R)-2,5-dimethylpiperazin-1-yl)quinazolin-6-yl)-2-methoxypyridin-3-yl)-2,4-difluorobenzenesulfonamide trifluoroacetate was obtained; yield: 98.0%; ESI-MS(m/z): 541.2 [M+H]⁺.

### Step d): Preparation of N-(5-(4-((2S,5R)-4-acryloyl-2,5-dimethylpiperazin-1-yl)quinazolin-6-yl)-2-methoxypyridin-3-yl)-2,4-difluorobenzenesulfonamide

N-(5-(4-((2S,5R)-2,5-dimethylpiperazin-1-yl)quinazolin-6-yl)-2-methoxypyridin-3-yl)-2,4-difluorobenzenesulfonamide trifluoroacetate (100.0 mg, 0.153 mmol) was dissolved in tetrahydrofuran (2 mL). The reaction system was cooled to −70° C. with N,N-diisopropylethylamine (118 mg, 0.918 mmol), acrylic acid (12 mg, 0.153 mmol) and T₃P (194 mg, 0.306 mmol) added successively. After the addition was completed, the reaction mixture was kept at −70° C. and stirred for 0.5 h. Upon completion of the reaction, the product was purified by Prep-HPLC to obtained N-(5-(4-((2S,5R)-4-acryloyl-2,5-dimethylpiperazin-1-yl)quinazolin-6-yl)-2-methoxypyridin-3-yl)-2,4-difluorobenzenesulfonamide, yield: 18.9%; ¹H NMR (400 MHz, DMSO-d₆) δ 10.34 (brs, 1H), 8.65 (s, 1H), 8.46 (s, 1H), 8.09-8.08 (m, 2H), 8.00 (s, 1H), 7.92 (d, J=8.0 Hz, 1H), 7.81-7.75 (m, 1H), 7.59-7.54 (m, 1H), 7.24-7.20 (m, 1H), 6.84-6.77 (m, 1H), 6.18 (d, J=16.0 Hz, 1H), 5.74 (d, J=12.0 Hz, 1H), 4.79-4.44 (m, 2H), 4.12-4.08 (m, 2H), 3.88-3.80 (m, 2H), 3.69 (s, 3H), 1.30-1.15 (m, 6H); ESI-MS(m/z): 595.0 [M+H]⁺.

### Example 15

### Preparation of N-(5-(4-((2S,5R)-2,5-dimethyl-4-((E)-4-oxopent-2-enoyl)piperazin-1-yl)quinazolin-6-yl)-2-methoxypyridin-3-yl)-2,4-difluorobenzenesulfonamide

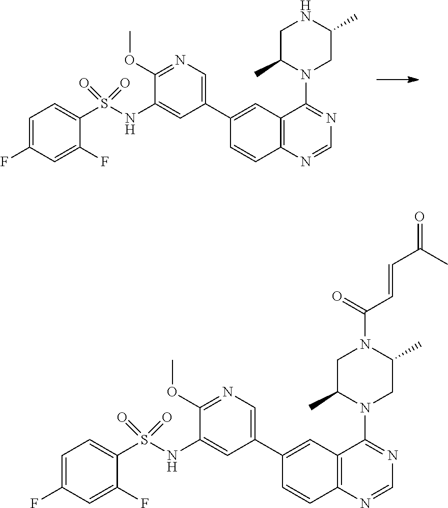 [SMILES: COc1ncc(-c2ccc3ncnc(N4C[C@@H](C)NC[C@@H]4C)c3c2)cc1NS(=O)(=O)c1ccc(F)cc1F ; COc1ncc(-c2ccc3ncnc(N4C[C@@H](C)N(C(=O)/C=C/C(C)=O)C[C@@H]4C)c3c2)cc1NS(=O)(=O)c1ccc(F)cc1F]

N-(5-(4-((2S,5R)-2,5-dimethylpiperazin-1-yl)quinazolin-6-yl)-2-methoxypyridin-3-yl)-2,4-difluorobenzenesulfonamide trifluoroacetate (100.0 mg, 0.157 mmol) was added to tetrahydrofuran (2 mL). The reaction system was cooled to −70° C., with N,N-diisopropylethylamine (121 mg, 0.942 mmol), 3-acetylacrylic acid (18 mg, 0.157 mmol) and T₃P (180 mg, 0.283 mmol) added successively. After the addition was completed, the reaction mixture was kept at −70° C. and stirred for 1 h. Upon completion of the reaction, the product was purified by Prep-HPLC. N-(5-(4-((2S,5R)-2,5-dimethyl-4-((E)-4-oxopent-2-enoyl)piperazin-1-yl)quinazolin-6-yl)-2-methoxypyridin-3-yl)-2,4-difluorobenzenesulfonamide was obtained, yield: 29.0%; ¹H NMR (400 MHz, DMSO-d₆) δ 10.34 (brs, 1H), 8.66 (s, 1H), 8.47 (s, 1H), 8.10-8.07 (m, 2H), 8.02-8.00 (m, 1H), 7.93 (d, J=8.0 Hz, 1H), 7.81-7.75 (m, 1H), 7.59-7.55 (m, 1H), 7.49 (d, J=16.0 Hz, 1H), 7.25-7.20 (m, 1H), 6.78 (d, J=16.0 Hz, 1H), 4.86-4.48 (m, 2H), 4.21-4.10 (m, 2H), 3.90-3.83 (m, 2H), 3.69 (s, 3H), 2.38 (s, 3H), 1.31-1.17 (m, 6H); ESI-MS(m/z): 637.0 [M+H]⁺.

### Example 16

### Preparation of (E)-2,4-difluoro-N-(5-(4-(4-(4-fluorobut-2-enoyl)piperazin-1-yl)quinazolin-6-yl)-2-methoxypyridin-3-yl)benzenesulfonamide

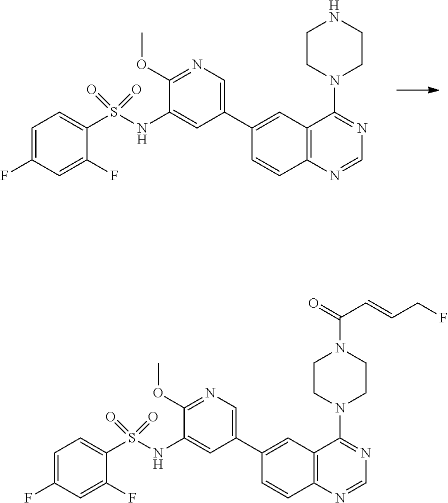 [SMILES: COc1ncc(-c2ccc3ncnc(N4CCNCC4)c3c2)cc1NS(=O)(=O)c1ccc(F)cc1F ; COc1ncc(-c2ccc3ncnc(N4CCN(C(=O)/C=C/CF)CC4)c3c2)cc1NS(=O)(=O)c1ccc(F)cc1F]

2,4-difluoro-N-(2-methoxy-5-(4-(piperazin-1-yl)quinazolin-6-yl)pyridin-3-yl)benzenesulfonamide trifluoroacetate (70 mg, 0.112 mmol) was added to tetrahydrofuran (2 mL), in the presense of protective nitrogen. The reaction system was cooled to −70° C. with N,N-diisopropylethylamine (87 mg, 0.670 mmol), (E)-4-fluorobut-2-enoic acid (12 mg, 0.112 mmol) and T₃P (142 mg, 0.224 mmol) added successively. After the addition was completed, the reaction mixture was kept at −70° C. and stirred for 0.5 h. Upon completion of the reaction, the reaction mixture was filtered and purified by Prep-HPLC. (f)-2,4-difluoro-N-(5-(4-(4-(4-fluorobut-2-enoyl)piperazin-1-yl)quinazolin-6-yl)-2-methoxypyridin-3-yl)benzenesulfonamide was obtained, yield: 37.4%; ¹H NMR (400 MHz, DMSO-d₆) δ 10.33 (brs, 1H), 8.65 (s, 1H), 8.46 (s, 1H), 8.13-8.07 (m, 2H), 8.01 (s, 1H), 7.92-7.90 (d, J=8.0 Hz, 1H), 7.80-7.74 (m, 1H), 7.58-7.53 (m, 1H), 7.23-7.18 (m, 1H), 6.85-6.79 (m, 1H), 6.77-6.71 (m, 1H), 5.20 (d, J=4.0 Hz, 1H), 5.09 (d, J=4.0 Hz, 1H), 3.88-3.78 (m, 8H), 3.68 (s, 3H); ESI-MS(m/z): 599.0 [M+H]⁺.

### Example 17

### Preparation of (E)-2,4-difluoro-N-(2-methoxy-5-(4-(4-(4-oxohept-2-enoyl)piperazin-1-yl)quinazolin-6-yl)pyridin-3-yl)benzenesulfonamide

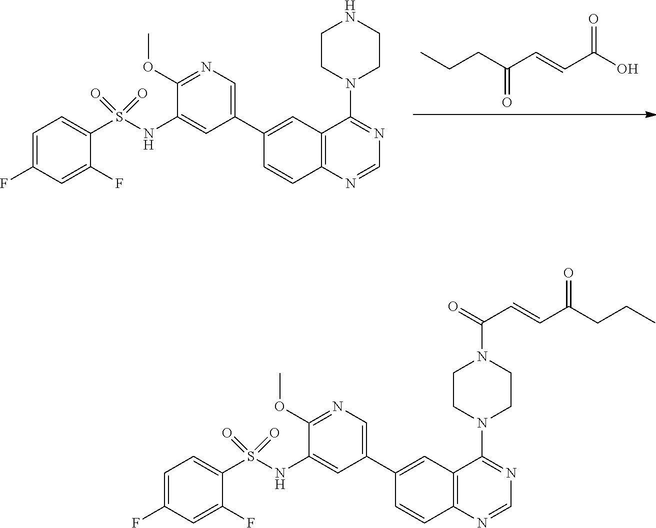

2,4-difluoro-N-(2-methoxy-5-(4-(piperazin-1-yl)quinazolin-6-yl)pyridin-3-yl)benzenesulfonamide trifluoroacetate (80 mg, 0.128 mmol) was added to tetrahydrofuran (2 mL). In the presense of protective nitrogen, the reaction system was cooled to −70° C., with N,N-diisopropylethylamine (99 mg, 0.768 mmol), (E)-4-oxohept-2-enoic acid (18 mg, 0.128 mmol) and T₃P (163 mg, 0.256 mmol) added successively. After the addition was completed, the reaction mixture was kept at −70° C. and stirred for 0.5 h. Upon completion of the reaction, the reaction mixture was filtered and purified by Prep-HPLC. (E)-2,4-difluoro-N-(2-methoxy-5-(4-(4-(4-oxohept-2-enoyl)piperazin-1-yl)quinazolin-6-yl)pyridin-3-yl)benzenesulfonamide was obtained, yield: 32.5%; ¹H NMR (400 MHz, DMSO-d₆) δ 10.33 (brs, 1H), 8.66 (s, 1H), 8.46 (s, 1H), 8.13-8.08 (m, 2H), 8.01 (d, J=4.0 Hz, 1H), 7.92 (d, J=8.0 Hz, 1H), 7.80-7.74 (m, 1H), 7.58-7.53 (m, 1H), 7.44 (d, J=16.0 Hz, 1H), 7.23-7.18 (m, 1H), 6.83 (d, J=16.0 Hz, 1H), 3.90-3.86 (m, 6H), 3.81-3.80 (m, 2H), 3.68 (s, 3H), 2.74-2.70 (m, 2H), 1.58-1.52 (m, 2H), 0.90-0.87 (m, 3H); ESI-MS(m/z): 637.0 [M+H]⁺.

### Example 18

### Preparation of N-(5-(4-(2-acryloyl-2,7-diazaspiro[3.5]nonan-7-yl)quinazolin-6-yl)-2-methoxypyridin-3-yl)-2,4-difluorobenzenesulfonamide

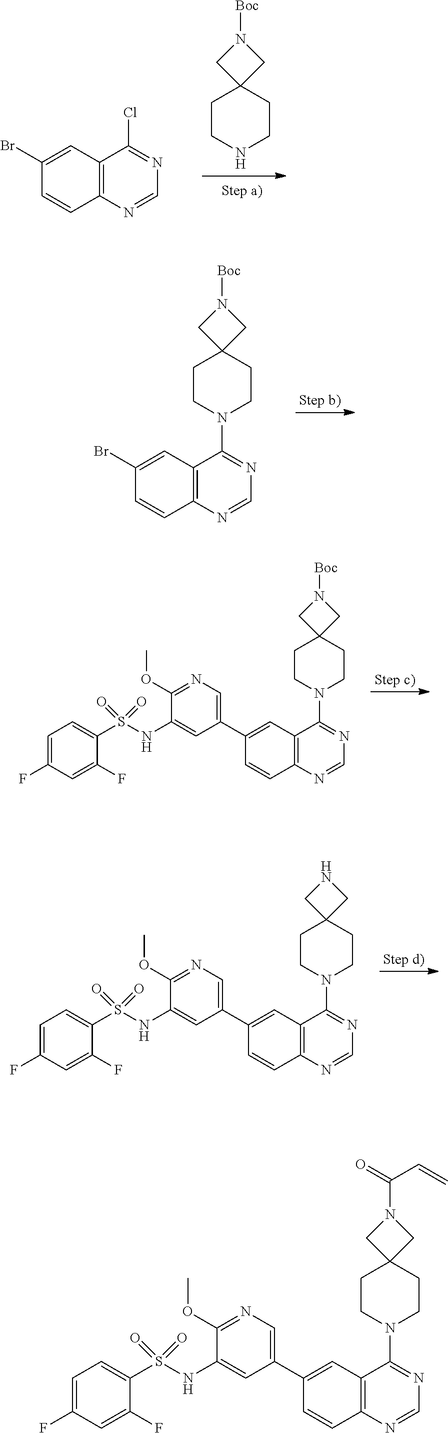 [SMILES: Clc1ncnc2ccc(Br)cc12 ; CC(C)(C)OC(=O)N1CC2(CCNCC2)C1 ; CC(C)(C)OC(=O)N1CC2(CCN(c3ncnc4ccc(Br)cc34)CC2)C1 ; COc1ncc(-c2ccc3ncnc(N4CCC5(CC4)CN(C(=O)OC(C)(C)C)C5)c3c2)cc1NS(=O)(=O)c1ccc(F)cc1F ; COc1ncc(-c2ccc3ncnc(N4CCC5(CC4)CNC5)c3c2)cc1NS(=O)(=O)c1ccc(F)cc1F ; C=CC(=O)N1CC2(CCN(c3ncnc4ccc(-c5cnc(OC)c(NS(=O)(=O)c6ccc(F)cc6F)c5)cc34)CC2)C1]

### Step a): Preparation of tert-butyl 7-(6-bromoquinazolin-4-yl)-2,7-diazaspiro[3.5]nonane-2-carboxylate

6-bromo-4-chloroquinazoline (600.0 mg, 2.480 mmol), tert-butyl 2,7-diazaspiro[3.5]nonane-2-carboxylate (560.9 mg, 2.480 mmol) were dissolved in dimethyl sulfoxide (30 mL), with triethylamine (501.1 mg, 4.960 mmol) added. After the addition was completed, the reaction mixture was heated to 60° C. and stirred for 2 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=2/1 to 1/1). Tert-butyl 7-(6-bromoquinazolin-4-yl)-2,7-diazaspiro[3.5]nonane-2-carboxylate was obtained, yield: 74.4%; ESI-MS(m/z): 433.3 [M+H]⁺.

### Step b): Preparation of tert-butyl 7-(6-(5-((2,4-difluorophenyl)sulfonamido)-6-methoxypyridin-3-yl)quinazolin-4-yl)-2,7-diazaspiro[3.5]nonane-2-carboxylate

Tert-butyl 7-(6-bromoquinazolin-4-yl)-2,7-diazaspiro[3.5]nonane-2-carboxylate (400.0 mg, 0.925 mmol), 2,4-difluoro-N-(2-hydroxy-5-(4,4,5,5-tetramethyl-1,3,2-dioxaborolan-2-yl)pyridin-3-yl)benzenesulfonamide (394.5 mg, 0.925 mmol), Pd(dppf)Cl₂ (135.4 mg, 0.185 mmol) and cesium carbonate (601.6 mg, 1.851 mmol) were added to dioxane/water mixed solvent (10 mL, v/v=10:1) successively. After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (100 mL/2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=2/1 to 1/1). Tert-butyl 7-(6-(5-((2,4-difluorophenyl)sulfonamido)-6-methoxypyridin-3-yl)quinazolin-4-yl)-2,7-diazaspiro[3.5]nonane-2-carboxylate was obtained, yield: 33.1%; ESI-MS(m/z): 653.7 [M+H]J.

### Step c): Preparation of N-(5-(4-(2,7-diazaspiro[3.5]nonan-7-yl)quinazolin-6-yl)-2-methoxypyridin-3-yl)-2,4-difluorobenzenesulfonamide

Tert-butyl 7-(6-(5-((2,4-difluorophenyl)sulfonamido)-6-methoxypyridin-3-yl)quinazolin-4-yl)-2,7-diazaspiro[3.5]nonane-2-carboxylate (200.0 mg, 0.306 mmol) was dissolved in dichloromethane (2 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. N-(5-(4-(2,7-diazaspiro[3.5]nonan-7-yl)quinazolin-6-yl)-2-methoxypyridin-3-yl)-2,4-difluorobenzenesulfonamide was obtained. The product can be used directly in the next reaction without purification. ESI-MS(m/z): 553.6 [M+H]⁺.

### Step d): Preparation of N-(5-(4-(2-acryloyl-2,7-diazaspiro[3.5]nonan-7-yl)quinazolin-6-yl)-2-methoxypyridin-3-yl)-2,4-difluorobenzenesulfonamide

N-(5-(4-(2,7-diazaspiro[3.5]nonan-7-yl)quinazolin-6-yl)-2-methoxypyridin-3-yl)-2,4-difluorobenzenesulfonamide (60.0 mg, 0.109 mmol) was dissolved in tetrahydrofuran (2 mL). The reaction system was cooled to −78° C., with N,N-diisopropylethylamine (70.1 mg, 0.543 mmol), acrylic acid (7.8 mg, 0.109 mmol) and 50% T₃P ethyl acetate solution (69.4 mg, 0.218 mmol) added successively After the addition was completed, the reaction mixture was kept at −78° C. and stirred for 1 h. Upon completion of the reaction, the reaction solution was diluted by adding acetonitrile (1 mL) and purified by Prep-HPLC. N-(5-(4-(2-acryloyl-2,7-diazaspiro[3.5]nonan-7-yl)quinazolin-6-yl)-2-methoxypyridin-3-yl)-2,4-difluorobenzenesulfonamide was obtained; yield: 10.1%; ¹H NMR (400 MHz, DMSO-d₆) δ 10.25 (s, 1H), 8.62 (s, 1H), 8.38 (s, 1H), 8.07-8.02 (m, 2H), 7.96 (s, 2H), 7.89-7.88 (m, 1H), 7.55 (s, 1H), 7.21 (d, J=4.0 Hz, 1H), 6.33-6.31 (m, 1H), 6.14 (s, 1H), 5.69-5.66 (m, 1H), 4.03 (s, 2H), 3.75-3.70 (m, 9H), 1.96-1.93 (m, 4H); ESI-MS(m/z): 607.6 [M+H]⁺.

### Example 19

### Preparation of 2,4-difluoro-N-(5-(4-(2-(2-fluoroacryloyl)-2,7-diazaspiro[3.5]nonan-7-yl)quinazolin-6-yl)-2-methoxypyridin-3-yl)benzenesulfonamide

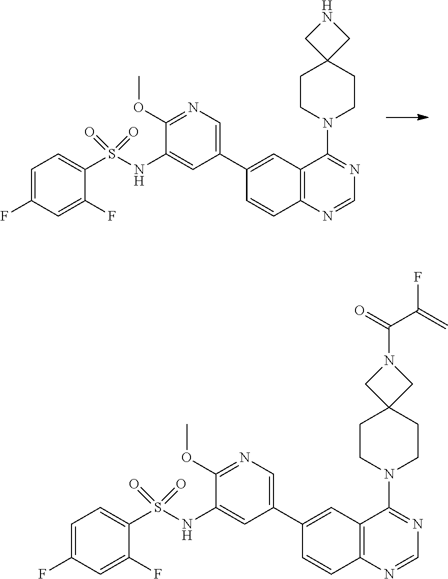 [SMILES: COc1ncc(-c2ccc3ncnc(N4CCC5(CC4)CNC5)c3c2)cc1NS(=O)(=O)c1ccc(F)cc1F ; C=C(F)C(=O)N1CC2(CCN(c3ncnc4ccc(-c5cnc(OC)c(NS(=O)(=O)c6ccc(F)cc6F)c5)cc34)CC2)C1]

N-(5-(4-(2,7-diazaspiro[3.5]nonan-7-yl)quinazolin-6-yl)-2-methoxypyridin-3-yl)-2,4-difluorobenzenesulfonamide (60.0 mg, 0.109 mmol) was dissolved in tetrahydrofuran (2 mL). The reaction system was cooled to −78° C., with N,N-diisopropylethylamine (70.1 mg, 0.543 mmol), 2-fluoroacrylic acid (9.8 mg, 0.109 mmol) and 50% T₃P ethyl acetate solution (69.4 mg, 0.218 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC. 2,4-difluoro-N-(5-(4-(2-(2-fluoroacryloyl)-2,7-diazaspiro[3.5]nonan-7-yl)quinazolin-6-yl)-2-methoxypyridin-3-yl)benzenesulfonamide was obtained; yield: 19.1%; ¹H NMR (400 MHz, DMSO-d₆) δ 8.61 (s, 1H), 8.15 (s, 1H), 7.98-7.93 (m, 2H), 7.87-7.75 (m, 3H), 7.35 (s, 1H), 7.14-7.13 (m, 1H), 5.55-5.42 (m, 1H), 5.32-5.27 (m, 1H), 4.18 (d, J=4.0 Hz, 2H), 3.80-3.69 (m, 9H), 1.95 (t, J=8.0 Hz, 4H); ESI-MS(m/z): 625.6 [M+H]⁺.

### Example 20

### Preparation of (E)-2,4-difluoro-N-(2-methoxy-5-(4-(2-(4-oxopent-2-enoyl)-2,7-diazaspiro[3.5]nonan-7-yl)quinazolin-6-yl)pyridin-3-yl)benzenesulfonamide

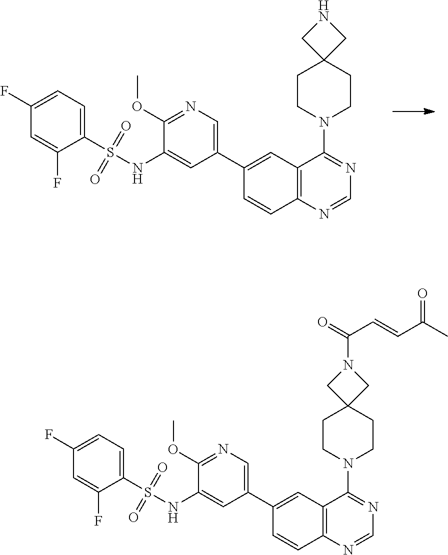 [SMILES: COc1ncc(-c2ccc3ncnc(N4CCC5(CC4)CNC5)c3c2)cc1NS(=O)(=O)c1ccc(F)cc1F ; COc1ncc(-c2ccc3ncnc(N4CCC5(CC4)CN(C(=O)/C=C/C(C)=O)C5)c3c2)cc1NS(=O)(=O)c1ccc(F)cc1F]

N-(5-(4-(2,7-diazaspiro[3.5]nonan-7-yl)quinazolin-6-yl)-2-methoxypyridin-3-yl)-2,4-difluorobenzenesulfonamide (60.0 mg, 0.109 mmol) was dissolved in tetrahydrofuran (2 mL). The reaction system was cooled to −78° C., with N,N-diisopropylethylamine (70.1 mg, 0.543 mmol), 3-acetylacrylic acid (12.4 mg, 0.109 mmol) and 50% T₃P ethyl acetate solution (69.4 mg, 0.218 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC. (E)-2,4-difluoro-N-(2-methoxy-5-(4-(2-(4-oxopent-2-enoyl)-2,7-diazaspiro[3.5]nonan-7-yl)quinazolin-6-yl)pyridin-3-yl)benzenesulfonamide was obtained; yield: 28.6%; ¹H NMR (40) MHz, DMSO-d₆) δ 10.35 (s, 1H), 8.63 (s, 1H), 8.4 (s, 1H), 8.09-8.07 (m, 2H), 8.05 (s, 1H), 7.99-7.91 (m, 1H), 7.89-7.78 (m, 1H), 7.57 (s, 1H), 7.22 (s, 1H), 6.93 (d, J=16.0 Hz, 1H), 6.75 (d, J=16.0 Hz, 1H), 4.15 (s, 2H), 3.80-3.70 (m, 9H), 2.35 (s, 3H), 1.97 (t, J=8.0 Hz, 4H); ESI-MS(m/z): 649.7 [M+H]⁺.

### Example 21

### Preparation of N-(5-(4-(4-acryloylpiperazin-1-yl)quinazolin-6-yl)-2-methoxypyridin-3-yl)-2,6-difluorobenzenesulfonamide

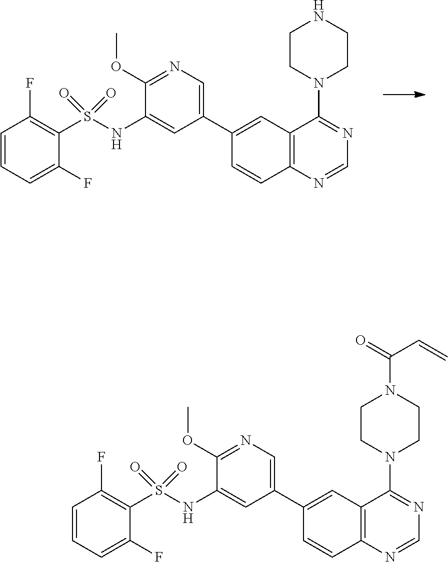 [SMILES: COc1ncc(-c2ccc3ncnc(N4CCNCC4)c3c2)cc1NS(=O)(=O)c1c(F)cccc1F ; C=CC(=O)N1CCN(c2ncnc3ccc(-c4cnc(OC)c(NS(=O)(=O)c5c(F)cccc5F)c4)cc23)CC1]

2,6-difluoro-N-(2-methoxy-5-(4-(piperazin-1-yl)quinazolin-6-yl)pyridin-3-yl)benzenesulfonamide (100.0 mg, 0.195 mmol) was dissolved in dichloromethane (10 mL). The reaction system was cooled to −78° C., with N,N-diisopropylethylamine (126.7 mg, 0.975 mmol), acrylic acid (21.1 mg, 0.293 mmol) and HATU (111.1 mg, 0.293 mmol) added successively. After the addition was completed, the reaction mixture was kept at −78° C. and stirred for 1 h. Upon completion of the reaction. The reaction was quenched by adding water (5 mL). The reaction mixture was extracted by adding dichloromethane (10 mL×2 times). The organic phases were combined, dried over anhydrous sodium sulfate and filtered. The filtrate was concentrated under reduced pressure. The crude product obtained was purified by Prep-HPLC. N-(5-(4-(4-acryloylpiperazin-1-yl)quinazolin-6-yl)-2-methoxypyridin-3-yl)-2,6-difluorobenzenesulfonamide was obtained, yield: 15.0%; ¹H NMR (400 MHz, DMSO-d₆) δ 10.61 (s, 1H), 8.67 (s, 1H), 8.50 (s, 1H), 8.15 (s, 1H), 8.14-8.11 (m, 1H), 8.11 (s, 1H), 7.92 (d, J=8 Hz, 1H), 7.75-7.71 (m, 1H), 7.31-7.26 (m, 2H), 6.21 (d, J=20 MHz, 1H), 6.87-6.81 (m, 1H), 5.75 (d, J=12.0 MHz, 1H), 3.90-3.79 (m, 8H), 3.65 (s, 3H); ESI-MS(m/z): 567.0 [M+H]⁺.

### Example 22

### Preparation of (E)-2,6-difluoro-N-(2-methoxy-5-(4-(4-(4-oxopent-2-enoyl)piperazin-1-yl)quinazolin-6-yl)pyridin-3-yl)benzenesulfonamide

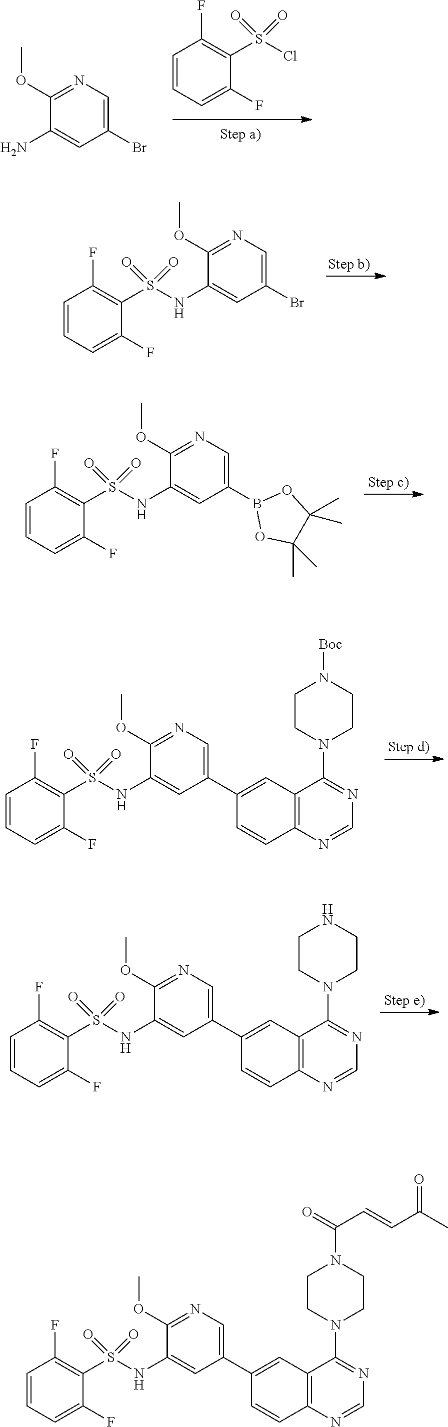

### Step a): Preparation of N-(5-bromo-2-methoxypyridin-3-yl)-2,6-difluorobenzenesulfonamide

5-bromo-2-methoxypyridin-3-amine (500 mg, 2.463 mmol), 2,6-difluorobenzenesulfonyl chloride (783.3 mg, 3.695 mmol), 4-dimethylaminopyridine (15.0 mg, 0.123 mmol) and pyridine (746.3 mg, 7.389 mmol) were dissolved in dichloromethane (20 mL). The reaction mixture was stirred at room temperature overnight. Upon completion of the reaction, the reaction was quenched by adding water (50 mL). The reaction mixture was extracted by adding dichloromethane (100 mL/2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=3/1 to 1/1), was obtained N-(5-bromo-2-methoxypyridin-3-yl)-2,6-difluorobenzenesulfonamide, yield: 69.7%; ESI-MS(m/z): 379.3 [M+H]⁺.

### Step b): Preparation of 2,6-difluoro-N-(2-methoxy-5-(4,4,5,5-tetramethyl-1,3,2-dioxaborolan-2-yl)pyridin-3-yl)benzenesulfonamide

N-(5-bromo-2-methoxypyridin-3-yl)-2,6-difluorobenzenesulfonamide (650 mg, 1.715 mmol), bis(pinacolato)diboron (653.4 mg, 2.573 mmol), Pd(dppf)Cl₂ (125.5 mg, 0.172 mmol) and potassium acetate (504.2 mg, 5.145 mmol) were dissolved in dioxane (30 mL). After the addition was completed, in the presense of protective nitrogen, the reaction mixture was heated to 110° C. and stirred for 2 h. Upon completion of the reaction, the reaction was quenched by adding water (50 mL). The reaction mixture was extracted with ethyl acetate (100 mL×2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=1/1 to 1/2), 2,6-difluoro-N-(2-methoxy-5-(4,4,5,5-tetramethyl-1,3,2-dioxaborolan-2-yl)pyridin-3-yl)benzenesulfonamide was obtained, yield: 95.8%; ESI-MS(m/z): 427.1 [M+H]⁺.

### Step c): Preparation of tert-butyl 4-(6-(5-((2,6-difluorophenyl)sulfonamido)-6-methoxypyridin-3-yl)quinazolin-4-yl)piperazine-1-carboxylate

2,6-difluoro-N-(2-methoxy-5-(4,4,5,5-tetramethyl-1,3,2-dioxaborolan-2-yl)pyridin-3-yl)benzenesulfonamide (400 mg, 0.939 mmol), tert-butyl 4-(6-bromoquinazolin-4-yl)piperazine-1-carboxylate (553.5 mg, 1.409 mmol), Pd(dppf)Cl₂ (68.7 mg, 0.094 mmol) and cesium carbonate (918.3 g, 2.817 mmol) were dissolved in dioxane/water (4:1.10 mL). After the addition was completed, the reaction mixture was heated to 110° C. and stirred for 3 h. Upon completion of the reaction, the reaction was quenched by adding water (50 mL). The reaction mixture was extracted with ethyl acetate (100 mL×2). The organic phases were combined, washed with saturated brine (100 mL 1), dried over anhydrous sodium sulfate and filtered. The filtrate was concentrated under reduced pressure. The crude product obtained was purified by silica gel column chromatography (Eluent: petroleum ether/ethyl acetate=2/1 to 1/1). Tert-butyl 4-(6-(5-((2,6-difluorophenyl)sulfonamido)-6-methoxypyridin-3-yl)quinazolin-4-yl)piperazine-1-carboxylate was obtained, yield: 69.4%; ESI-MS(m/z): 613.2 [M+H]⁺.

### Step d): Preparation of 2,6-difluoro-N-(2-methoxy-5-(4-(piperazin-1-yl)quinazolin-6-yl)pyridin-3-yl)benzenesulfonamide

Tert-butyl 4-(6-(5-((2,6-difluorophenyl)sulfonamido)-6-methoxypyridin-3-yl)quinazolin-4-yl)piperazine-1-carboxylate (400 mg, 0.435 mmol) was dissolved in dichloromethane (4 mL). Under the ice bath condition, trifluoroacetic acid (2 mL) was slowly added dropwise. After the addition was completed, the reaction mixture was stirred at room temperature for 1 h. Upon completion of the reaction, the reaction mixture was concentrated under reduced pressure. 2,6-difluoro-N-(2-methoxy-5-(4-(piperazin-1-yl)quinazolin-6-yl)pyridin-3-yl)benzenesulfonamide was obtained. ESI-MS(m/z): 513.1[M+H]⁺.

### Step e): Preparation of (E)-2,6-difluoro-N-(2-methoxy-5-(4-(4-(4-oxopent-2-enoyl)piperazin-1-yl)quinazolin-6-yl)pyridin-3-yl)benzenesulfonamide

2,6-difluoro-N-(2-methoxy-5-(4-(piperazin-1-yl)quinazolin-6-yl)pyridin-3-yl)benzenesulfonamide (100.0 mg, 0.195 mmol) was dissolved in tetrahydrofuran (10 mL). The reaction system was cooled to −78° C., with N,N-diisopropylethylamine (126.7 mg, 0.975 mmol), (E)-4-oxopent-2-enoic acid (33.3 mg, 0.293 mmol) and T₃P (186.0 mg, 0.293 mmol) added successively. After the addition was completed, the reaction mixture was kept at −78° C. and stirred for 1 h. Upon completion of the reaction, the reaction was quenched by adding water (5 mL). The reaction mixture was extracted by adding dichloromethane (10 mL/2 times). The organic phases were combined, dried over anhydrous sodium sulfate and filtered. The filtrate was concentrated under reduced pressure. The crude product obtained was purified by Prep-HPLC. (E)-2,6-difluoro-N-(2-methoxy-5-(4-(4-(4-oxopent-2-enoyl)piperazin-1-yl)quinazolin-6-yl)pyridin-3-yl)benzenesulfonamide was obtained, yield: 9.8%; ¹H NMR (400 MHz, DMSO-d₆) δ 10.55 (s, 1H), 8.67 (s, 1H), 8.44 (s, 1H), 8.13-8.10 (m, 2H), 8.09 (s, 1H), 7.92 (d, J=8 Hz, 1H), 7.71-7.68 (m, 1H), 7.45 (d, J=16 Hz, 1H), 7.28-7.23 (m, 2H), 6.78-6.73 (m, 1H), 3.90-3.80 (m, 8H), 3.63 (s, 3H), 2.37 (s, 3H); ESI-MS(m/z): 609.0 [M+H]⁺.

### Example 23

### Preparation of N-(5-(4-(4-acryloylpiperazin-1-yl)quinazolin-6-yl)-2-methoxypyridin-3-yl)-2,4-difluorobenzamide

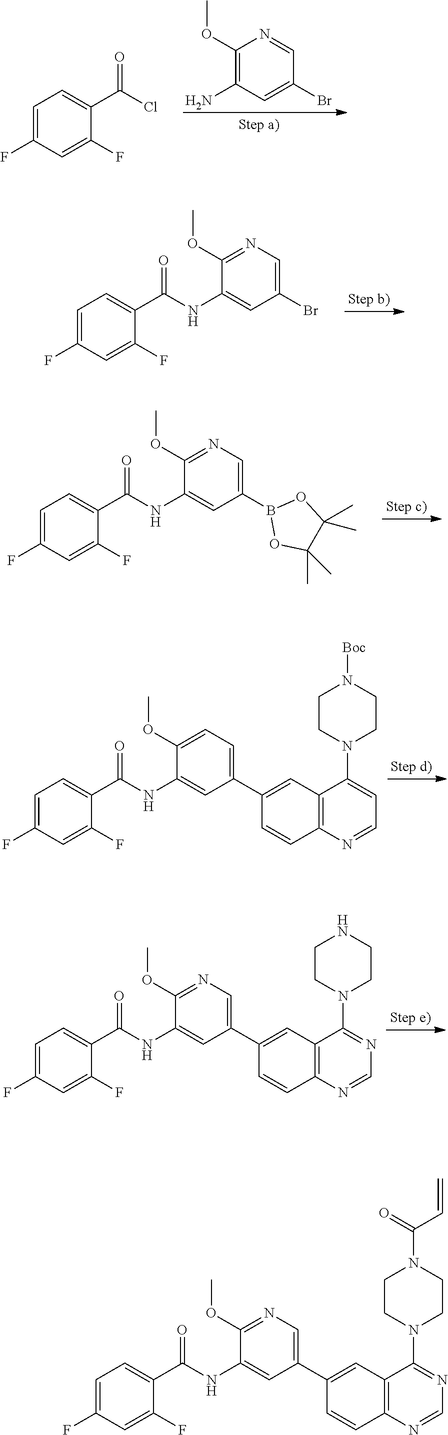

### Step a): Preparation of N-(5-bromo-2-methoxypyridin-3-yl)-2,4-difluorobenzamide

2,4-difluorobenzoyl chloride (3.0 g, 11.111 mmol) was dissolved in dichloromethane (10 mL). Under the ice bath condition, triethylamine (3.0 g, 11.111 mmol) and 5-bromo-2-methoxypyridin-3-amine (3.0 g, 11.111 mmol) were added to the system. After the addition was completed, the reaction mixture was heated to room temperature and stirred overnight. Upon completion of the reaction, the reaction was quenched by adding water (50 mL). The reaction mixture was extracted by adding dichloromethane (100 mL×2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=3/1 to 1/1). N-(5-bromo-2-methoxypyridin-3-yl)-2,4-difluorobenzamide was obtained, yield: 51.3%; ESI-MS(m/z): 343.1 [M+H]⁺.

### Step b): Preparation of 2,4-difluoro-N-(2-methoxy-5-(4,4,5,5-tetramethyl-1,3,2-dioxaborolan-2-yl)pyridin-3-yl)benzamide

N-(5-bromo-2-methoxypyridin-3-yl)-2,4-difluorobenzamide (500 mg, 1.458 mmol), bis(pinacolato)diboron (555.4 mg, 2.187 mmol), Pd(dppf)Cl₂ (106.7 mg, 0.146 mmol) and potassium acetate (428.6 mg, 4.374 mmol) were dissolved in dioxane (30 mL). After the addition was completed, in the presense of protective nitrogen, the reaction mixture was heated to 110° C. and stirred for 2 h. Upon completion of the reaction. The reaction was quenched by adding water (50 mL). The reaction mixture was extracted with ethyl acetate (100 mL×2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=1/1 to 1/2), 2,4-difluoro-N-(2-methoxy-5-(4,4,5,5-tetramethyl-1,3,2-dioxaborolan-2-yl)pyridin-3-yl)benzamide was obtained, yield: 87.9%; ESI-MS(m/z): 391.3 [M+H]⁺.

### Step c): Preparation of tert-butyl 4-(6-(3-(2,4-difluorobenzamido)-4-methoxyphenyl)quinolin-4-yl)piperazine-1-carboxylate

2,4-difluoro-N-(2-methoxy-5-(4,4,5,5-tetramethyl-1,3,2-dioxaborolan-2-yl)pyridin-3-yl)benzamide (400 mg, 1.026 mmol), tert-butyl 4-(6-bromoquinazolin-4-yl)piperazine-1-carboxylate (603.1 mg, 1.539 mmol), Pd(dppf)Cl₂ (75.1 mg, 0.103 mmol) and cesium carbonate (1.0 g, 3.078 mmol) were dissolved in dioxane/water (4:1, 10 mL). After the addition was completed, the reaction mixture was heated to 110° C. and stirred for 3 h. Upon completion of the reaction. The reaction was quenched by adding water (50 mL). The reaction mixture was extracted with ethyl acetate (100 mL×2). The organic phases were combined, washed with saturated brine (100 mL 1), dried over anhydrous sodium sulfate and filtered. The filtrate was concentrated under reduced pressure. The crude product obtained was purified by silica gel column chromatography (Eluent: petroleum ether/ethyl acetate=2/1 to 1/1). Tert-butyl 4-(6-(3-(2,4-difluorobenzamido)-4-methoxyphenyl)quinolin-4-yl)piperazine-1-carboxylate was obtained, yield: 42.4%; ESI-MS(m/z): 577.2 [M+H]⁺.

### Step d): Preparation of 2,4-difluoro-N-(2-methoxy-5-(4-(piperazin-1-yl)quinazolin-6-yl)pyridin-3-yl)benzamide

Tert-butyl 4-(6-(3-(2,4-difluorobenzamido)-4-methoxyphenyl)quinolin-4-yl)piperazine-1-carboxylate (250 mg, 0.435 mmol) was dissolved in dichloromethane (4 mL). Under the ice bath condition, trifluoroacetic acid (2 mL) was slowly added dropwise. After the addition was completed, the reaction mixture was stirred at room temperature for 1 h. Upon completion of the reaction, the reaction mixture was concentrated under reduced pressure. 2,4-difluoro-N-(2-methoxy-5-(4-(piperazin-1-yl)quinazolin-6-yl)pyridin-3-yl)benzamide was obtained. The product can be used directly in the next reaction without further purification. ESI-MS(m/z): 477.3 [M+H]⁺.

### Step e): Preparation of N-(5-(4-(4-acryloylpiperazin-1-yl)quinazolin-6-yl)-2-methoxypyridin-3-yl)-2,4-difluorobenzamide

2,4-difluoro-N-(2-methoxy-5-(4-(piperazin-1-yl)quinazolin-6-yl)pyridin-3-yl)benzamide (150.0 mg, 0.314 mmol) was dissolved in dichloromethane (10 mL). The reaction system was cooled to −78° C., with N,N-diisopropylethylamine (204.4 mg, 1.570 mmol), acrylic acid (34.0 mg, 0.471 mmol) and HATU (179.2 mg, 0.471 mmol) added successively. After the addition was completed, the reaction mixture was kept at −78° C. and stirred for 1 h. Upon completion of the reaction. The reaction was quenched by adding water (5 mL). The reaction mixture was extracted by adding dichloromethane (10 mL×2 times). The organic phases were combined, dried over anhydrous sodium sulfate and filtered. The filtrate was concentrated under reduced pressure. The crude product obtained was purified by Prep-HPLC. N-(5-(4-(4-acryloylpiperazin-1-yl)quinazolin-6-yl)-2-methoxypyridin-3-yl)-2,4-difluorobenzamide was obtained, yield: 54.9%; ¹H NMR (400 MHz, DMSO-d₆) δ 9.09-9.04 (m, 2H), 8.70 (s, 1H), 8.18-8.13 (m, 2H), 8.05-8.98 (m, 3H), 7.04-7.00 (m, 1H), 6.94-6.89 (m, 1H), 6.60-6.53 (m, 1H), 6.33-6.28 (m, 1H), 5.72 (d, J=12.0 Hz, 1H), 4.08 (s, 3H), 3.88-3.82 (m, 8H); ESI-MS(m/z): 531.0 [M+H]⁺.

### Example 24

### Preparation of N-(5 (4-(4-acryloylpiperazin-1-yl)quinazolin-6-yl)-2-methoxypyridin-3-yl)benzenesulfonamide

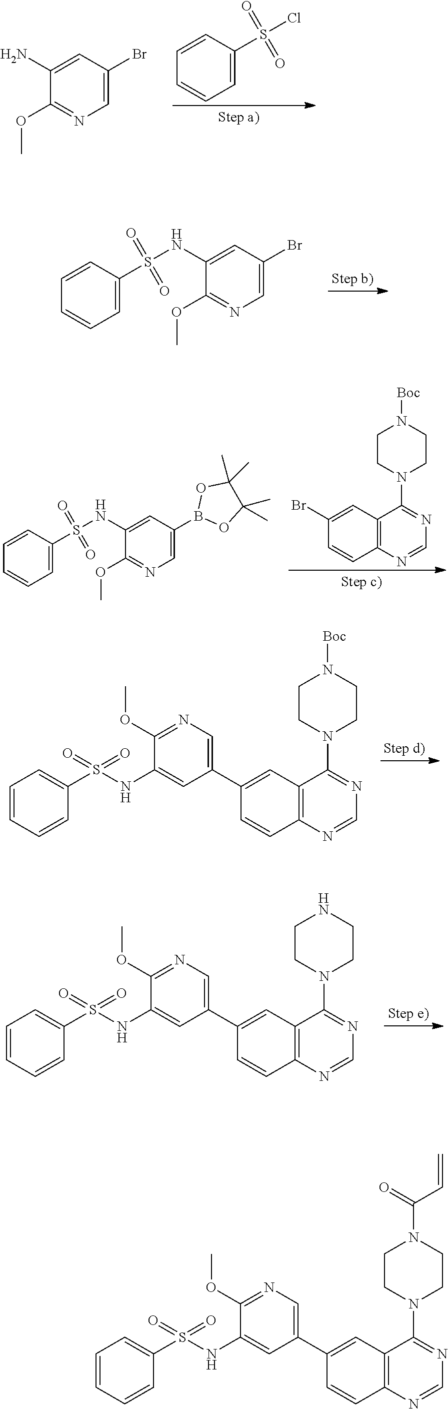

### Step a): Preparation of N-(5-bromo-2-methoxypyridin-3-yl)benzenesulfonamide

5-bromo-2-methoxypyridin-3-amine (600 mg, 2.970 mmol), benzenesulfonyl chloride (522.8 mg, 2.970 mmol), 4-dimethylaminopyridine (36.3 mg, 0.297 mmol), Pyridine (352.5 mg, 4.456 mmol) and dichloromethane (12 mL) were added to the reaction flask successively. After the addition was completed, the reaction mixture was stirred at room temperature for 4 h. Upon completion of the reaction, the reaction was quenched by adding water (50 mL). The reaction system was extracted with dichloromethane (100 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=2/1 to 1/1). N-(5-bromo-2-methoxypyridin-3-yl)benzenesulfonamide was obtained, yield: 49.2%; ESI-MS(m/z): 343.2 [M+H]⁺.

### Step b): Preparation of N-(2-methoxy-5-(4,4,5,5-tetramethyl-1,3,2-dioxaborolan-2-yl)pyridin-3-yl)benzenesulfonamide

N-(5-bromo-2-methoxypyridin-3-yl)benzenesulfonamide (600.0 mg, 1.755 mmol), bis(pinacolato)diboron (534.7 mg, 1.755 mmol). Pd(dppf)Cl₂ (256.5 mg, 0.351 mmol) and potassium acetate (792.1 mg, 8.070 mmol) were dissolved in dioxane (12 mL) successively. After the addition was completed, in the presense of protective nitrogen, the reaction mixture was kept at 100° C. and stirred for 4 h. Upon completion of the reaction, the reaction mixture was filtered. The filtrate was concentrated under reduced pressure. The crude product obtained was purified by silica gel column chromatography (Eluent: petroleum ether/ethyl acetate=2/1 to 1/1). N-(2-methoxy-5-(4,4,5,5-tetramethyl-1,3,2-dioxaborolan-2-yl)pyridin-3-yl)benzenesulfonamide was obtained, yield: 87.7%; ESI-MS(m/z): 391.2 [M+H]⁺.

### Step c): Preparation of tert-butyl 4-(6-(6-methoxy-5-(phenylsulfonamido)pyridin-3-yl)quinazolin-4-yl)piperazine-1-carboxylate

Tert-butyl 4-(6-bromoquinazolin-4-yl)piperazine-1-carboxylate (490.0 mg, 1.256 mmol), N-(2-methoxy-5-(4,4,5,5-tetramethyl-1,3,2-dioxaborolan-2-yl)pyridin-3-yl)benzenesulfonamide (492.2 mg, 1.256 mmol), Pd(dppf)Cl₂ (275.5 mg, 0.377 mmol) and cesium carbonate (780.3 mg, 2.512 mmol) were added to dioxane/water mixed solvent (10 mL, v/v=10:1). After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (100 mL×2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=2/1). Tert-butyl 4-(6-(6-methoxy-5-(phenylsulfonamido)pyridin-3-yl)quinazolin-4-yl)piperazine-1-carboxylate was obtained, yield: 23.5%; ESI-MS(m/z): 577.2 [M+H]⁺.

### Step d): Preparation of N-(2-methoxy-5-(4-(piperazin-1-yl)quinazolin-6-yl)pyridin-3-yl)benzenesulfonamide

Tert-butyl 4-(6-(6-methoxy-5-(phenylsulfonamido)pyridin-3-yl)quinazolin-4-yl)piperazine-1-carboxylate (75.0 mg, 0.130 mmol) was dissolved in dichloromethane (4 mL). Under the ice bath condition, trifluoroacetic acid (2 mL) was slowly added dropwise. After the addition was completed, the reaction mixture was stirred at room temperature for 1 h. Upon completion of the reaction, the reaction mixture was concentrated under reduced pressure. N-(2-methoxy-5-(4-(piperazin-1-yl)quinazolin-6-yl)pyridin-3-yl)benzenesulfonamide was obtained. The product can be used directly in the next reaction without further purification. ESI-MS(m/z): 477.6 [M+H]⁺.

### Step e): Preparation of N-(5-(4-(4-acryloylpiperazin-1-yl)quinazolin-6-yl)-2-methoxypyridin-3-yl)benzenesulfonamide

N-(2-methoxy-5-(4-(piperazin-1-yl)quinazolin-6-yl)pyridin-3-yl)benzenesulfonamide (62.0 mg, 0.130 mmol) was dissolved in dichloromethane (2 mL). The reaction system was cooled to −78° C., with N,N-diisopropylethylamine (134.3 mg, 1.041 mmol), acrylic acid (9.4 mg, 0.130 mmol) and HATU (74.2 mg, 0.195 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC. N-(5-(4-(4-acryloylpiperazin-1-yl)quinazolin-6-yl)-2-methoxypyridin-3-yl)benzenesulfonamide was obtained, yield: 10.1%; ¹H NMR (400 MHz, DMSO-d₆) δ 10.07 (s, 1H), 8.78 (s, 1H), 8.44 (s, 1H), 8.21 (s, 2H), 7.99-7.94 (m, 2H), 7.79-7.77 (m, 2H), 7.65-7.55 (m, 3H), 6.81 (s, 1H), 6.18 (d, J=4.0 Hz, 1H), 5.78 (d, J=4.0 Hz, 1H), 4.01-4.08 (m, 4H), 3.88-3.81 (m, 4H), 3.67 (s, 3H); ESI-MS(m/z): 531.6 [M+H]⁺.

### Example 25

### Preparation of N-(5-(4-(4-acryloylpiperazin-1-yl)quinazolin-6-yl)-2-methoxypyridin-3-yl)cyclohexanesulfonamide

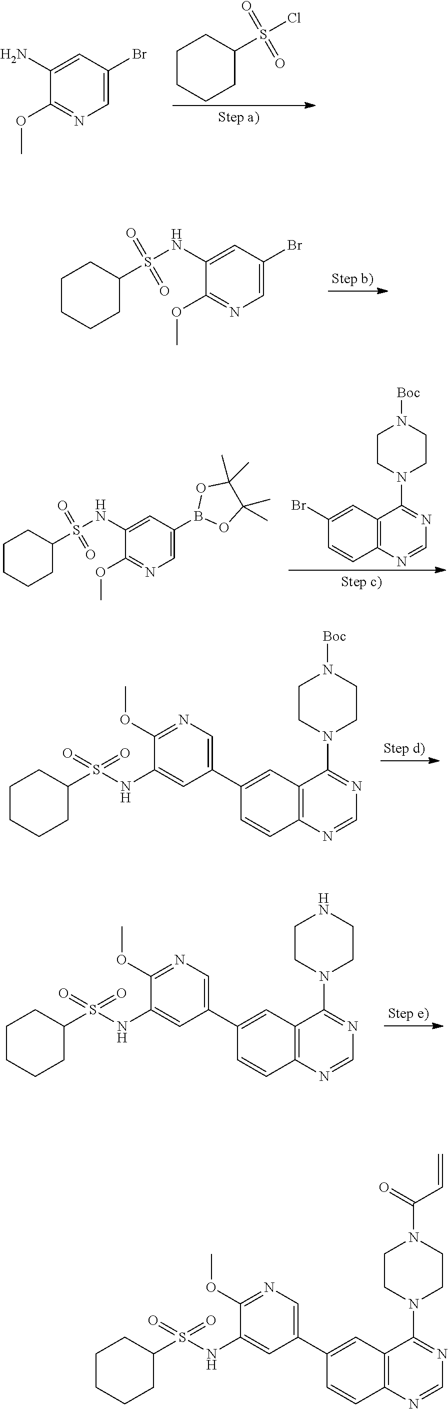

### Step a): Preparation of N-(5-bromo-2-methoxypyridin-3-yl)cyclohexanesulfonamide

5-bromo-2-methoxypyridin-3-amine (500 mg, 2.476 mmol) and tetrahydrofuran (10 mL) were added successively to the reaction flask. Potassium bistrimethylsilylamide (5.0 mL, 4.951 mmol) was slowly added to the system. After the addition was completed, the mixture was stirred for 30 min, with cyclohexanesulfonyl chloride (675.9 mg, 3.713 mmol) added. After the addition was completed, the reaction mixture was stirred at room temperature for 12 h. Upon completion of the reaction, the reaction was quenched by adding water (50 mL) slowly. The reaction system was extracted with ethyl acetate (50 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=3/1 to 1/1). N-(5-bromo-2-methoxypyridin-3-yl)cyclohexanesulfonamide was obtained, yield: 58.0%; ESI-MS(m/z): 349.2 [M+H]⁺.

### Step b): Preparation of N-(2-methoxy-5-(4,4,5,5-tetramethyl-1,3,2-dioxaborolan-2-yl)pyridin-3-yl)cyclohexanesulfonamide

N-(5-bromo-2-methoxypyridin-3-yl)cyclohexanesulfonamide (500 mg, 1.437 mmol), bis(pinacolato)diboron (437.8 mg, 1.724 mmol), Pd(dppf)Cl_{z}(210.0 mg, 0.287 mmol) and potassium acetate (647.7 mg, 6.610 mmol) were added to dioxane (10 mL) successively. After the addition was completed, the reaction mixture was stirred at 100° C. for 4 h under an atmosphere of nitrogen. Upon completion of the reaction, the reaction mixture was filtered. The filtrate was concentrated under reduced pressure. The crude product obtained was purified by silica gel column chromatography (Eluent: petroleum ether/ethyl acetate=2/1 to 1/1). N-(2-methoxy-5-(4,4,5,5-tetramethyl-1,3,2-dioxaborolan-2-yl)pyridin-3-yl)cyclohexanesulfonamide was obtained, yield: 70.3%; ESI-MS(m/z): 396.2 [M+H]⁺.

### Step c): Preparation of tert-butyl 4-(6-(5-(cyclohexanesulfonamido)-6-methoxypyridin-3-yl)quinazolin-4-yl)piperazine-1-carboxylate

Tert-butyl 4-(6-bromoquinazolin-4-yl)piperazine-1-carboxylate (395.9 mg, 1.010 mmol), N-(2-methoxy-5-(4,4,5,5-tetramethyl-1,3,2-dioxaborolan-2-yl)pyridin-3-yl)cyclohexanesulfonamide (400 mg, 1.010 mmol)) Pd(dppf)Cl₂ (147.6 mg, 0.202 mmol) and cesium carbonate (656.2 mg, 2.020 mmol) were added to dioxane/water mixed solvent (8 mL, v/v=10:1). After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (100 mL×2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=2/1 to 0/1). Tert-butyl 4-(6-(5-(cyclohexanesulfonamido)-6-methoxypyridin-3-yl)quinazolin-4-yl)piperazine-1-carboxylate was obtained, yield: 34.0%; ESI-MS(m/z): 583.2 [M+H]⁺.

### Step d): Preparation of N-(2-methoxy-5-(4-(piperazin-1-yl)quinazolin-6-yl)pyridin-3-yl)cyclohexanesulfonamide

Tert-butyl 4-(6-(5-(cyclohexanesulfonamido)-6-methoxypyridin-3-yl)quinazolin-4-yl)piperazine-1-carboxylate (80.0 mg, 0.137 mmol) was dissolved in dichloromethane (2 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. N-(2-methoxy-5-(4-(piperazin-1-yl)quinazolin-6-yl)pyridin-3-yl)cyclohexanesulfonamide was obtained.

The product can be used directly in the next reaction without further purification. ESI-MS(m/z): 483.1 [M+H]⁺.

### Step e): Preparation of N-(5-(4-(4-acryloylpiperazin-1-yl)quinazolin-6-yl)-2-methoxypyridin-3-yl)cyclohexanesulfonamide

N-(2-methoxy-5-(4-(piperazin-1-yl)quinazolin-6-yl)pyridin-3-yl)cyclohexanesulfonamide (60 mg, 0.124 mmol) was dissolved in tetrahydrofuran (2 mL). The reaction system was cooled to −78° C. with N,N-diisopropylethylamine (96 mg, 0.744 mmol), acrylic acid (8.9 mg, 0.124 mmol) and 50% T₃P ethyl acetate solution (79.1 mg, 0.248 mmol) added successively. After the addition was completed, the reaction mixture was stirred at −78° C. for 1 h. Upon completion of the reaction, the reaction was quenched by adding acetonitrile (0 mL). The reaction solution was purified by Prep-HPLC. N-(5-(4-(4-acryloylpiperazin-1-yl)quinazolin-6-yl)-2-methoxypyridin-3-yl)cyclohexanesulfonamide was obtained, yield: 40.7%; ¹H NMR (400 MHz, DMSO-d₆) δ 9.36 (s, 1H), 8.66 (s, 1H), 8.45 (s, 1H), 8.13-8.11 (m, 2H), 8.02 (d, J=4.0 Hz, 1H), 7.92 (d, J=8.0 Hz, 1H), 6.81 (t, J=16.0 Hz, 1H), 6.19-6.14 (m, 1H), 5.75-5.72 (m, 1H), 3.99 (s, 3H), 3.89-3.79 (m, 8H), 3.10 (s, 1H), 2.13 (d, J=12.0 Hz, 1H), 1.80-1.77 (m, 2H), 1.62 (d, J=12.0 Hz, 1H), 1.44-1.37 (m, 2H), 1.31-1.21 (m, 2H), 1.18-1.12 (m, 1H); ESI-MS(m/z): 537.5 [M+H]⁺.

### Example 26

### Preparation of (E)-N-(2-methoxy-5-(4-(4-(4-oxopent-2-enoyl)piperazin-1-yl)quinazolin-6-yl)pyridin-3-yl)cyclohexanesulfonamide

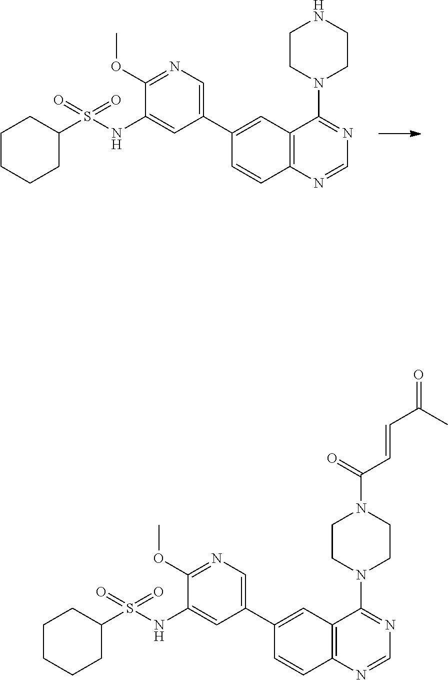 [SMILES: COc1ncc(-c2ccc3ncnc(N4CCNCC4)c3c2)cc1NS(=O)(=O)C1CCCCC1 ; COc1ncc(-c2ccc3ncnc(N4CCN(C(=O)/C=C/C(C)=O)CC4)c3c2)cc1NS(=O)(=O)C1CCCCC1]

(E)-N-(2-methoxy-5-(4-(4-(4-oxopent-2-enoyl)piperazin-1-yl)quinazolin-6-yl)pyridin-3-yl)cyclohexanesulfonamide (60.0 mg, 0.124 mmol) was dissolved in tetrahydrofuran (2 mL). The reaction system was cooled to −78° C., with N,N-diisopropylethylamine (96.0 mg, 0.744 mmol), 3-acetylacrylic acid (14.1 mg, 0.124 mmol) and 50% T₃P ethyl acetate solution (79.1 mg, 0.248 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC. (E)-N-(2-methoxy-5-(4-(4-(4-oxopent-2-enoyl)piperazin-1-yl)quinazolin-6-yl)pyridin-3-yl)cyclohexanesulfonamide was obtained, yield: 45.9%; ¹H NMR (400 MHz, DMSO-d₆) δ 9.35 (s, 1H), 8.67 (s, 1H), 8.45 (s, 1H), 8.14-8.11 (m, 2H), 8.02 (d, J=4.0 Hz, 1H), 7.92 (d, J=8.0 Hz, 1H), 7.44 (d, J=16.0 Hz, 1H), 6.73 (d, J=16.0 Hz, 1H), 3.99 (s, 3H), 3.89-3.80 (m, 8H), 3.10 (s, 1H), 2.37 (s, 3H), 2.13 (d, J=16.0 Hz, 2H), 1.62-1.59 (m, 1H), 1.80-1.77 (m, 2H), 1.44-1.40 (m, 2H), 1.27-1.24 (m, 2H), 1.18-1.12 (m, 1H); ESI-MS(m/z): 579.2 [M+H]⁺.

### Example 27

### Preparation of N-(5-(4-(4-acryloylpiperazin-1-yl)quinazolin-6-yl)pyridin-3-yl)-2,4-difluorobenzenesulfonamide

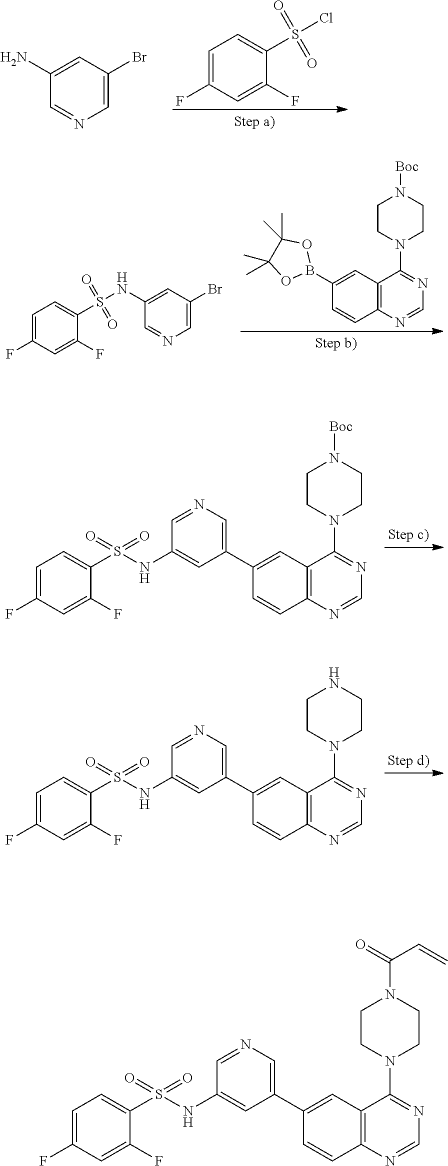

### Step a): Preparation of N-(5-bromopyridin-3-yl)-2,4-difluorobenzenesulfonamide

5-bromopyridin-3-amine (600.0 mg, 3.489 mmol), 2,4-difluorobenzenesulfonyl chloride (739.5 mg, 3.489 mmol), 4-dimethylaminopyridine (42.6 mg, 0.349 mmol), pyridine (413.5 mg, 5.233 mmol) and dichloromethane (12 mL) were added to the reaction flask successively. After the addition was completed, the reaction mixture was stirred at room temperature for 4 h. Upon completion of the reaction, the reaction was quenched by adding water (50 mL). The reaction system was extracted with dichloromethane (100 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=3/1 to 1/1). N-(5-bromopyridin-3-yl)-2,4-difluorobenzenesulfonamide was obtained, yield: 65.9%; ESI-MS(m/z): 349.2 [M+H]⁺.

### Step b): Preparation of tert-butyl 4-(6-(5-((2,4-difluorophenyl)sulfonamido)pyridin-3-yl)quinazolin-4-yl)piperazine-1-carboxylate

Tert-butyl 4-(6-(4,4,5,5-tetramethyl-1,3,2-dioxaborolan-2-yl)quinazolin-4-yl)piperazine-1-carboxylate (252.4 mg, 0.573 mmol), N-(5-bromopyridin-3-yl)-2,4-difluorobenzenesulfonamide (200 mg, 0.573 mmol), Pd(dppf)Cl₂ (167.4 mg, 0.115 mmol) and cesium carbonate (372.3 mg, 1.145 mmol) were added to dioxane/water mixed solvent (10 mL, v/v=10:1). After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (100 mL×2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=2/1 to 1/1). Tert-butyl 4-(6-(5-((2,4-difluorophenyl)sulfonamido)pyridin-3-yl)quinazolin-4-yl)piperazine-1-carboxylate was obtained, yield: 39.0%; ESI-MS(m/z): 583.6 [M+H]⁺.

### Step c): Preparation of 2,4-difluoro-N-(5-(4-(piperazin-1-yl)quinazolin-6-yl)pyridin-3-yl)benzenesulfonamide

Tert-butyl 4-(6-(5-((2,4-difluorophenyl)sulfonamido)pyridin-3-yl)quinazolin-4-yl)piperazine-1-carboxylate (130.0 mg, 0.223 mmol) was dissolved in dichloromethane (4 mL). Under the ice bath condition, trifluoroacetic acid (4 mL) was slowly added dropwise. After the addition was completed, the reaction mixture was stirred at room temperature for 1 h. Upon completion of the reaction, the reaction mixture was concentrated under reduced pressure. 2,4-difluoro-N-(5-(4-(piperazin-1-yl)quinazolin-6-yl)pyridin-3-yl)benzenesulfonamide was obtained.

The product can be used directly in the next reaction without further purification. ESI-MS(m/z): 483.1 [M+H]⁺.

### Step d): Preparation of N-(5-(4-(4-acryloylpiperazin-1-yl)quinazolin-6-yl)pyridin-3-yl)-2,4-difluorobenzenesulfonamide

2,4-difluoro-N-(5-(4-(piperazin-1-yl)quinazolin-6-yl)pyridin-3-yl)benzenesulfonamide (60.0 mg, 0.124 mmol) was dissolved in tetrahydrofuran (2 mL). The reaction system was cooled to −78° C. with N,N-diisopropylethylamine (96.0 mg, 0.744 mmol), acrylic acid (8.9 mg, 0.124 mmol) and 50% T₃P ethyl acetate solution (79.1 mg, 0.248 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC. N-(5-(4-(4-acryloylpiperazin-1-yl)quinazolin-6-yl)pyridin-3-yl)-2,4-difluorobenzenesulfonamide was obtained, yield: 12.0%; ¹H NMR (400 MHz, DMSO-d₆) δ 11.2 (s, 1H), 8.78-8.77 (m, 2H), 8.39-8.38 (m, 1H), 8.24 (s, 1H), 8.13-8.01 (m, 1H), 7.95-7.86 (m, 2H), 7.57 (s, 1H), 7.30 (s, 1H), 6.83-6.78 (m, 1H), 6.21-6.17 (m, 1H), 5.77-5.74 (m, 1H), 4.09-4.06 (m, 4H), 3.88-3.80 (m, 4H); ESI-MS(m/z): 537.5 [M+H]⁺.

### Example 28

### Preparation of (E)-2,4-difluoro-N-(5-(4-(4-(4-oxopent-2-enoyl)piperazin-1-yl)quinazolin-6-yl)pyridin-3-yl)benzenesulfonamide

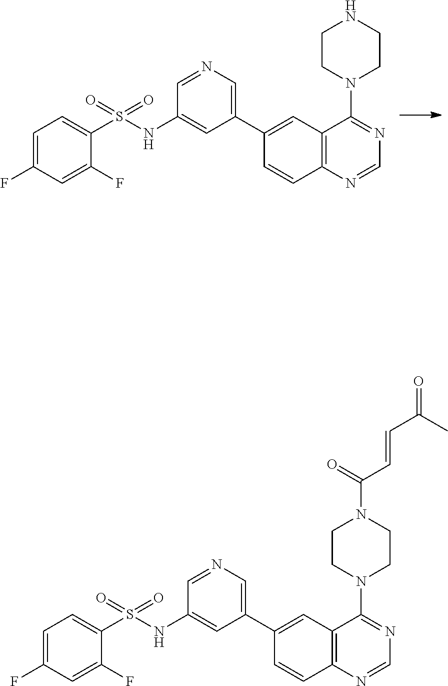 [SMILES: O=S(=O)(Nc1cncc(-c2ccc3ncnc(N4CCNCC4)c3c2)c1)c1ccc(F)cc1F ; CC(=O)/C=C/C(=O)N1CCN(c2ncnc3ccc(-c4cncc(NS(=O)(=O)c5ccc(F)cc5F)c4)cc23)CC1]

2,4-difluoro-N-(5-(4-(piperazin-1-yl)quinazolin-6-yl)pyridin-3-yl)benzenesulfonamide (60.0 mg, 0.124 mmol) was dissolved in tetrahydrofuran (2 mL). The reaction system was cooled to −78° C., with N,N-diisopropylethylamine (96.0 mg, 0.744 mmol), 3-acetylacrylic acid (14.1 mg, 0.124 mmol) and 50% T₃P ethyl acetate solution (79.1 mg, 0.248 mmol) added successively. After the addition was completed, the reaction mixture was kept at −78° C. and stirred for 1 h. Upon completion of the reaction, the reaction was quenched by adding acetonitrile (1 mL), the reaction solution was purified by Prep-HPLC. (E)-2,4-difluoro-N-(5-(4-(4-(4-oxopent-2-enoyl)piperazin-1-yl)quinazolin-6-yl)pyridin-3-yl)benzenesulfonamide was obtained, yield: 21.8%; H NMR (400 MHz, DMSO-d₆) δ 11.2 (s, 1H), 8.79-8.77 (m, 2H), 8.38-8.24 (m, 1H), 8.15 (d, J=8.0 Hz, 1H), 8.01 (s, 2H), 7.95 (d, J=8.0 Hz, 1H), 7.87 (s, 1H), 7.57 (s, 1H), 7.42 (d, J=16.0 Hz, 1H), 7.29 (s, 1H), 6.76 (d, J=16.0 Hz, 1H), 4.12-4.11 (m, 4H), 3.93-3.80 (m, 4H), 2.37 (s, 3H); ESI-MS(m/z): 579.5 [M+H]⁺.

### Example 29

### Preparation of N-(5-(4-(4-acryloylpiperazin-1-yl)-2-methylquinazolin-6-yl)-2-methoxypyridin-3-yl)-2,4-difluorobenzenesulfonamide

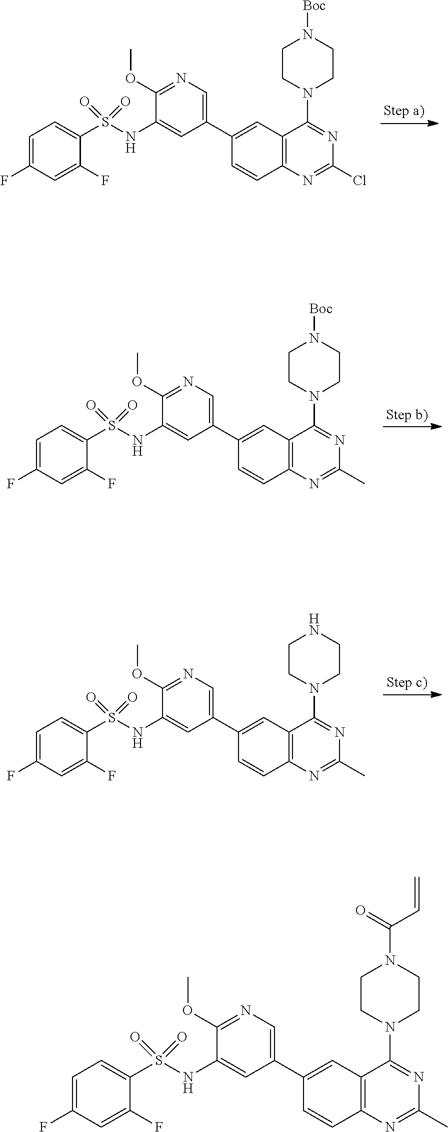 [SMILES: COc1ncc(-c2ccc3nc(Cl)nc(N4CCN(C(=O)OC(C)(C)C)CC4)c3c2)cc1NS(=O)(=O)c1ccc(F)cc1F ; COc1ncc(-c2ccc3nc(C)nc(N4CCN(C(=O)OC(C)(C)C)CC4)c3c2)cc1NS(=O)(=O)c1ccc(F)cc1F ; COc1ncc(-c2ccc3nc(C)nc(N4CCNCC4)c3c2)cc1NS(=O)(=O)c1ccc(F)cc1F ; C=CC(=O)N1CCN(c2nc(C)nc3ccc(-c4cnc(OC)c(NS(=O)(=O)c5ccc(F)cc5F)c4)cc23)CC1]

### Step a): Preparation of tert-butyl 4-(6-(5-((2,4-difluorophenyl)sulfonamido)-6-methoxypyridin-3-yl)-2-methylquinazolin-4-yl)piperazine-1-carboxylate

Tert-buty 4-(2-chloro-6-(5-((2,4-difluorophenyl)sulfonamido)-6-methoxypyridin-3-yl)quinazolin-4-yl)piperazine-1-carboxylate (200 mg, 0.309 mmol), pinacol methylborate (88 mg, 0.620 mmol), Pd(dppf)Cl₂ (23 mg, 0.031 mmol) and potassium carbonate (128 mg, 0.926 mmol) were added to N,N-dimethylformamide/water mixed solvent (5 mL, v/v=10:1). After the addition was completed, in the presense of protective nitrogen, the reaction mixture was heated to 100° C. and stirred overnight. Upon completion of the reaction, the reaction was quenched by adding water (30 mL). The reaction mixture was extracted with ethyl acetate (60 mL×2). The organic phases were combined, washed with saturated brine (50 mL×1), dried over anhydrous sodium sulfate and filtered. The filtrate was concentrated under reduced pressure. The crude product obtained was purified by silica gel column chromatography (Eluent: petroleum ether/ethyl acetate=2/1 to 1/1). Tert-butyl 4-(6-(5-((2,4-difluorophenyl)sulfonamido)-6-methoxypyridin-3-yl)-2-methylquinazolin-4-yl)piperazine-1-carboxylate was obtained, yield: 36.3%; ESI-MS(m/z): 627.20 [M+H]⁺.

### Step b): Preparation of 2,4-difluoro-N-(2-methoxy-5-(2-methyl-4-(piperazin-1-yl)quinazolin-6-yl)pyridin-3-yl)benzenesulfonamide

Tert-butyl 4-(6-(5-((2,4-difluorophenyl)sulfonamido)-6-methoxypyridin-3-yl)-2-methylquinazolin-4-yl)piperazine-1-carboxylate (70 mg, 0.112 mmol) was dissolved in dichloromethane (2 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. 2,4-difluoro-N-(2-methoxy-5-(2-methyl-4-(piperazin-1-yl)quinazolin-6-yl)pyridin-3-yl)benzenesulfonamide was obtained. The product can be used directly in the next reaction without further purification. ESI-MS(m/z): 527.2 [M+H]⁺.

### Step c): Preparation of N-(5-(4-(4-acryloylpiperazin-1-yl)-2-methylquinazolin-6-yl)-2-methoxypyridin-3-yl)-2,4-difluorobenzenesulfonamide

2,4-difluoro-N-(2-methoxy-5-(2-methyl-4-(piperazin-1-yl)quinazolin-6-yl)pyridin-3-yl)benzenesulfonamide (50 mg, 0.095 mmol) and acrylic acid (34 mg, 0.472 mmol) were dissolved in tetrahydrofuran (2 mL). The reaction system was cooled to −78° C., with N,N-diisopropylethylamine (61 mg, 0.472 mmol) and 1-propylphosphonic anhydride (91 mg, 0.286 mmol) added successively. After the addition was completed, the reaction mixture was kept at −78° C. and stirred for 1 h. Upon completion of the reaction, the reaction system was diluted by adding acetonitrile (1 mL). The reaction solution was purified by Prep-HPLC. N-(5-(4-(4-acryloylpiperazin-1-yl)-2-methylquinazolin-6-yl)-2-methoxypyridin-3-yl)-2,4-difluorobenzenesulfonamide was obtained, yield: 24.3%; ¹H NMR (400 MHz, DMSO-d₆) δ 10.3 (s, 1H), 8.42-8.38 (m, 1H), 8.04-7.93 (m, 3H), 7.79-7.69 (m, 2H), 7.56-7.49 (m, 1H), 7.19-7.14 (m, 1H), 6.83-6.75 (m, 1H), 6.16-6.09 (m, 1H), 5.72-5.66 (m, 1H), 3.81-3.71 (m, 8H), 3.63 (s, 3H), 2.51 (s, 3H); ESI-MS(m/z): 581.0 [M+H]⁺.

### Example 30

### Preparation of N-(5-(4-(4-acryloylpiperazin-1-yl)-2-aminoquinazolin-6-yl)-2-methoxypyridin-3-yl)-2,4-difluorobenzenesulfonamide

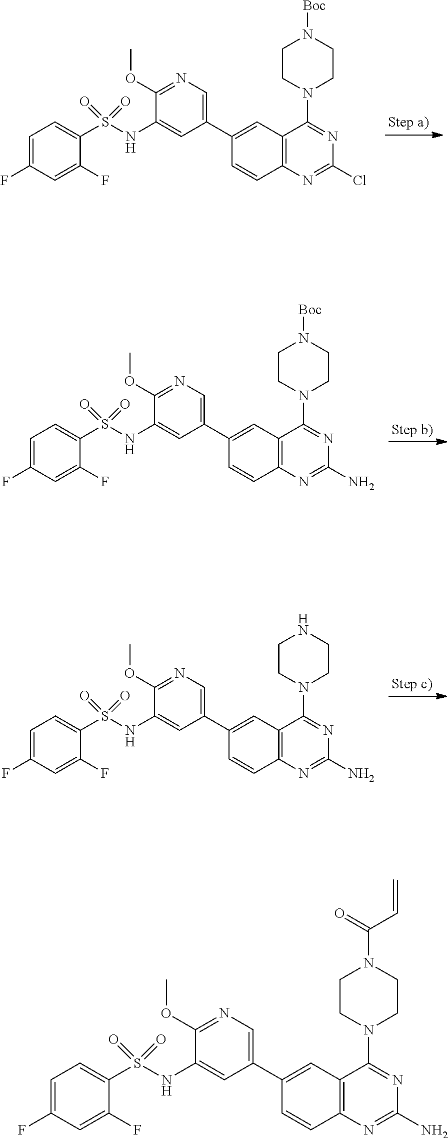 [SMILES: COc1ncc(-c2ccc3nc(Cl)nc(N4CCN(C(=O)OC(C)(C)C)CC4)c3c2)cc1NS(=O)(=O)c1ccc(F)cc1F ; COc1ncc(-c2ccc3nc(N)nc(N4CCN(C(=O)OC(C)(C)C)CC4)c3c2)cc1NS(=O)(=O)c1ccc(F)cc1F ; COc1ncc(-c2ccc3nc(N)nc(N4CCNCC4)c3c2)cc1NS(=O)(=O)c1ccc(F)cc1F ; C=CC(=O)N1CCN(c2nc(N)nc3ccc(-c4cnc(OC)c(NS(=O)(=O)c5ccc(F)cc5F)c4)cc23)CC1]

### Step a): Preparation of tert-butyl 4-(2-amino-6-(5-((2,4-difluorophenyl)sulfonamido)-6-methoxypyridin-3-yl)quinazolin-4-yl)piperazine-1-carboxylate

Tert-butyl 4-(2-chloro-6-(5-((2,4-difluorophenyl)sulfonamido)-6-methoxypyridin-3-yl)quinazolin-4-yl)piperazine-1-carboxylate (250 mg, 0.386 mmol) were dissolved in NH₃/methanol solution (6 mL), the reaction solution was placed in a scaled tube, heated to 100° C. and stirred overnight. Upon completion of the reaction, the reaction solution was purified by reversed-phase column chromatography (mobile phase: acetonitrile/water (0.1% TFA)=0 to 50%). Tert-butyl 4-(2-amino-6-(5-((2,4-difluorophenyl)sulfonamido)-6-methoxypyridin-3-yl)quinazolin-4-yl)piperazine-1-carboxylate was obtained, yield: 20.7%; ESI-MS(m/z): 628.2 [M+H]⁺.

### Step b): Preparation of N-(5-(2-amino-4-(piperazin-1-yl)quinazolin-6-yl)-2-methoxypyridin-3-yl)-2,4-difluorobenzenesulfonamide

Tert-butyl 4-(2-amino-6-(5-((2,4-difluorophenyl)sulfonamido)-6-methoxypyridin-3-yl)quinazolin-4-yl)piperazine-1-carboxylate (50 mg, 0.080 mmol) was dissolved in dichloromethane (2 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. N-(5-(2-amino-4-(piperazin-1-yl)quinazolin-6-yl)-2-methoxypyridin-3-yl)-2,4-difluorobenzenesulfonamide was obtained. The product can be used directly in the next reaction without further purification. ESI-MS(m/z): 528.2 [M+H]⁺.

### Step c): Preparation of N-(5-(4-(4-acryloylpiperazin-1-yl)-2-aminoquinazolin-6-yl)-2-methoxypyridin-3-yl)-2,4-difluorobenzenesulfonamide

N-(5-(2-amino-4-(piperazin-1-yl)quinazolin-6-yl)-2-methoxypyridin-3-yl)-2,4-difluorobenzenesulfonamide (40 mg, 0.076 mmol) was dissolved in dichloromethane (1.5 mL). The reaction system was cooled to −78° C., with N,N-diisopropylethylamine (49 mg, 0.379 mmol), acrylic acid (11 mg, 0.153 mmol) and HATU (35 mg, 0.092 mmol) added successively. After the addition was completed, the reaction mixture was kept at −78° C. and stirred for 1 h. Upon completion of the reaction, the reaction system was diluted by adding acetonitrile (1 mL). The reaction solution was purified by Prep-HPLC. N-(5-(4-(4-acryloylpiperazin-1-yl)-2-aminoquinazolin-6-yl)-2-methoxypyridin-3-yl)-2,4-difluorobenzenesulfonamide was obtained, yield: 4.3%; ¹H NMR (400 MHz, DMSO-d₆) δ 11.84 (s, 1H), 10.26 (s, 1H), 9.25-8.95 (m, 1H), 8.55-8.46 (m, 1H), 8.44-8.40 (m, 1H), 8.17-8.06 (m, 1H), 8.05-8.02 (m, 1H), 7.74-7.60 (m, 2H), 7.56-7.49 (m, 1H), 7.18-7.12 (m, 1H), 6.89-6.78 (m, 1H), 6.18-6.10 (m, 1H), 5.73-5.69 (m, 1H), 3.85 (s, 4H), 3.77-3.65 (m, 4H), 3.57 (s, 3H); ESI-MS(m/z): 582.0 [M+H]⁺.

### Example 31

### Preparation of N-(5-(4-(4-acryloylpiperazin-1-yl)-2-cyanoquinazolin-6-yl)-2-methoxypyridin-3-yl)-2,4-difluorobenzenesulfonamide

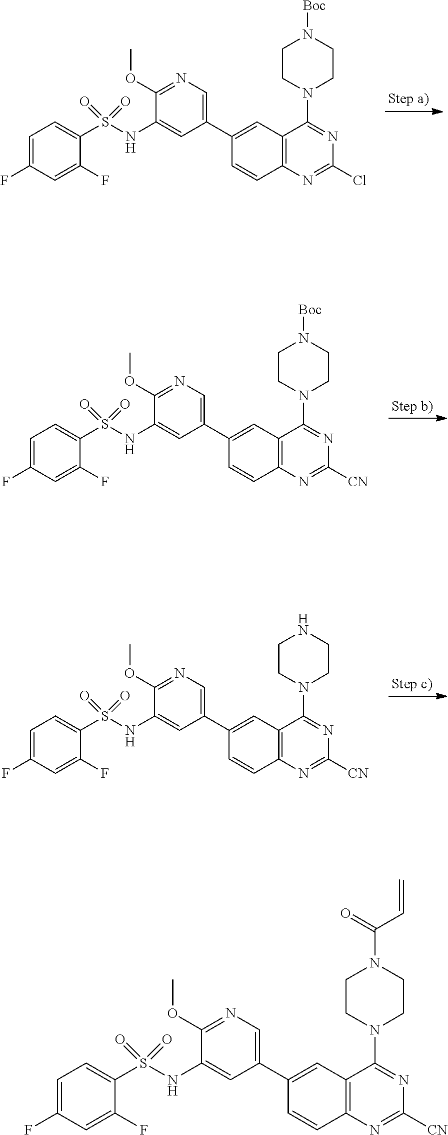 [SMILES: COc1ncc(-c2ccc3nc(Cl)nc(N4CCN(C(=O)OC(C)(C)C)CC4)c3c2)cc1NS(=O)(=O)c1ccc(F)cc1F ; COc1ncc(-c2ccc3nc(C#N)nc(N4CCN(C(=O)OC(C)(C)C)CC4)c3c2)cc1NS(=O)(=O)c1ccc(F)cc1F ; COc1ncc(-c2ccc3nc(C#N)nc(N4CCNCC4)c3c2)cc1NS(=O)(=O)c1ccc(F)cc1F ; C=CC(=O)N1CCN(c2nc(C#N)nc3ccc(-c4cnc(OC)c(NS(=O)(=O)c5ccc(F)cc5F)c4)cc23)CC1]

### Step a): Preparation of tert-butyl 4-(2-cyano-6-(5-((2,4-difluorophenyl)sulfonamido)-6-methoxypyridin-3-yl)quinazolin-4-yl)piperazine-1-carboxylate

Tert-butyl 4-(2-chloro-6-(5-((2,4-difluorophenyl)sulfonamido)-6-methoxypyridin-3-yl)quinazolin-4-yl)piperazine-1-carboxylate (150 mg, 0.232 mmol), Zinc cyanide (55 mg, 0.464 mmol), Pd(dppf)Cl₂ (37 mg, 0.046 mmol) and cesium carbonate (227 mg, 0.696 mmol) were added to N-methylpyrrolidone (5 mL). After the addition was completed, under nitrogen atmosphere, the reaction mixture was microwave heated to 150° C. and reacted for 2 h. Upon completion of the reaction, the reaction was quenched by adding water (30 mL). The reaction mixture was extracted with ethyl acetate (100 mL/2 times). The organic phases were combined, washed with saturated brine (50 mL×1 time), dried over anhydrous sodium sulfate and filtered. The filtrate was concentrated under reduced pressure. The crude product obtained was purified by silica gel column chromatography (Eluent: petroleum ether/ethyl acetate=5/1 to 3/1). Tert-butyl 4-(2-cyano-6-(5-((2,4-difluorophenyl)sulfonamido)-6-methoxypyridin-3-yl)quinazolin-4-yl)piperazine-1-carboxylate was obtained, yield: 48.6%; ESI-MS(m/z): 638.2 [M+H]⁺.

### Step b): Preparation of N-(5-(2-cyano-4-(piperazin-1-yl)quinazolin-6-yl)-2-methoxypyridin-3-yl)-2,4-difluorobenzenesulfonamide

Tert-butyl 4-(2-cyano-6-(5-((2,4-difluorophenyl)sulfonamido)-6-methoxypyridin-3-yl)quinazolin-4-yl)piperazine-1-carboxylate (50 mg, 0.078 mmol) was dissolved in dichloromethane (2 mL). Under the ice bath condition, TFA (1 mL) was slowly added dropwise. After the addition was completed, the reaction mixture was stirred at room temperature for 1 h. Upon completion of the reaction, the reaction mixture was concentrated under reduced pressure. N-(5-(2-cyano-4-(piperazin-1-yl)quinazolin-6-yl)-2-methoxypyridin-3-yl)-2,4-difluorobenzenesulfonamide was obtained: 538.1 [M+H]⁺.

### Step c): Preparation of N-(5-(4-(4-acryloylpiperazin-1-yl)-2-cyanoquinazolin-6-yl)-2-methoxypyridin-3-yl)-2,4-difluorobenzenesulfonamide

N-(5-(2-cyano-4-(piperazin-1-yl)quinazolin-6-yl)-2-methoxypyridin-3-yl)-2,4-difluorobenzenesulfonamide (30 mg, 0.056 mmol) was dissolved in tetrahydrofuran (2 mL). The reaction system was cooled to −78° C., with N,N-diisopropylethylamine (36 mg, 0.280 mmol), acrylic acid (4 mg, 0.056 mmol) and 50% T₃P ethyl acetate solution (36 mg, 0.112 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC. N-(5-(4-(4-acryloylpiperazin-1-yl)-2-cyanoquinazolin-6-yl)-2-methoxypyridin-3-yl)-2,4-difluorobenzenesulfonamide was obtained, yield: 18.5%; ¹H NMR (400 MHz, DMSO-d₆) δ 10.36 (brs, 1H), 8.53-8.52 (m, 1H), 8.26-8.21 (m, 1H), 8.07-8.06 (m, 1H), 8.00-7.98 (m, 1H), 7.80-7.74 (m, 1H), 7.61-7.55 (m, 1H), 7.24-7.19 (m, 1H), 6.89-6.79 (m, 1H), 6.21-6.16 (m, 1H), 5.77-5.74 (m, 1H), 4.06-4.03 (m, 4H), 3.87-3.79 (m, 4H), 3.68 (s, 3H); ESI-MS(m/z): 592.2 [M+H]⁺.

### Example 32

### Preparation of N-(5-(4-(4-acryloylpiperazin-1-yl)-2-hydroxyquinazolin-6-yl)-2-methoxypyridin-3-yl)-2,4-difluorobenzenesulfonamide

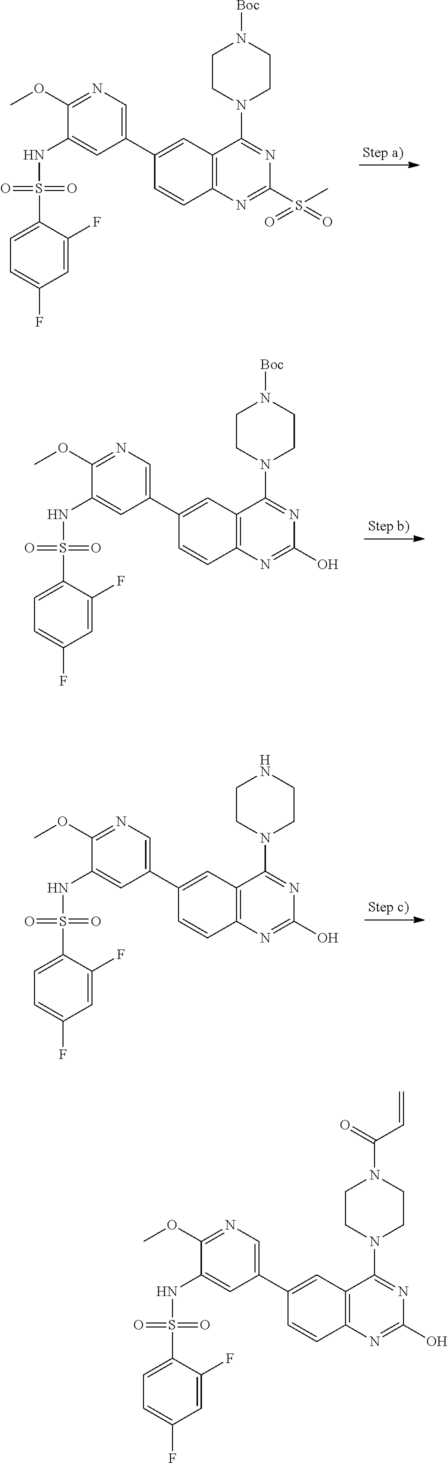 [SMILES: COc1ncc(-c2ccc3nc(S(C)(=O)=O)nc(N4CCN(C(=O)OC(C)(C)C)CC4)c3c2)cc1NS(=O)(=O)c1ccc(F)cc1F ; COc1ncc(-c2ccc3nc(O)nc(N4CCN(C(=O)OC(C)(C)C)CC4)c3c2)cc1NS(=O)(=O)c1ccc(F)cc1F ; COc1ncc(-c2ccc3nc(O)nc(N4CCNCC4)c3c2)cc1NS(=O)(=O)c1ccc(F)cc1F ; C=CC(=O)N1CCN(c2nc(O)nc3ccc(-c4cnc(OC)c(NS(=O)(=O)c5ccc(F)cc5F)c4)cc23)CC1]

### Step a): Preparation of tert-butyl 4-(6-(5-((2,4-difluorophenyl)sulfonamido)-6-methoxypyridin-3-yl)-2-hydroxyquinazolin-4-yl)piperazine-1-carboxylate

Tert-butyl 4-(6-(5-((2,4-difluorophenyl)sulfonamido)-6-methoxypyridin-3-yl)-2-(methylsulfonyl)quinazolin-4-yl)piperazine-1-carboxylate (170.0 mg, 0.246 mmol), potassium hydroxide (55.3 mg, 0.985 mmol) were dissolved in dimethyl sulfoxide (10 mL) and water (2 mL) successively. The reaction mixture was heated to 100° C. and stirred for 2 h. Upon completion of the reaction, the reaction solution was adjust to pH=2-3 with 1M hydrogen chloride aqueous solution and extracted with dichloromethane (10 mL×3). The organic phase was concentrated under reduced pressure. Tert-butyl 4-(6-(5-((2,4-difluorophenyl)sulfonamido)-6-methoxypyridin-3-yl)-2-hydroxyquinazolin-4-yl)piperazine-1-carboxylate was obtained, crude product yield: 99.9%; ESI-MS(m/z): 629.6 [M+H]⁺.

### Step b): Preparation of 2,4-difluoro-N-(5-(2-hydroxy-4-(piperazin-1-yl)quinazolin-6-yl)-2-methoxypyridin-3-yl)benzenesulfonamide

Tert-butyl 4-(6-(5-((2,4-difluorophenyl)sulfonamido)-6-methoxypyridin-3-yl)-2-hydroxyquinazolin-4-yl)piperazine-1-carboxylate (170.0 mg, 0.072 mmol) was dissolved in dichloromethane (4 mL). Under the ice bath condition, trifluoroacetic acid (2 mL) was slowly added dropwise. After the addition was completed, the reaction mixture was stirred at room temperature for 1 h. Upon completion of the reaction, the reaction mixture was concentrated under reduced pressure. 2,4-difluoro-N-(5-(2-hydroxy-4-(piperazin-1-yl)quinazolin-6-yl)-2-methoxypyridin-3-yl)benzenesulfonamide was obtained. The product can be used directly in the next reaction without further purification. ESI-MS(m/z): 529.5 [M+H]⁺.

### Step c): Preparation of N-(5-(4-(4-acryloylpiperazin-1-yl)-2-hydroxyquinazolin-6-yl)-2-methoxypyridin-3-yl)-2,4-difluorobenzenesulfonamide

2,4-difluoro-N-(5-(2-hydroxy-4-(piperazin-1-yl)quinazolin-6-yl)-2-methoxypyridin-3-yl)benzenesulfonamide (80.0 mg, 0.151 mmol) was dissolved in dichloromethane (5 mL). The reaction system was cooled to −78° C., with N,N-diisopropylethylamine (97.7 mg, 0.757 mmol), acrylic acid (10.9 mg, 0.151 mmol) and HATU (69.1 mg, 0.182 mmol) added successively. After the addition was completed, the reaction mixture was kept at −78° C. and stirred for 1 h. Upon completion of the reaction, acetonitrile (1 mL) was added for dissolution. The reaction solution was purified by Prep-HPLC. N-(5-(4-(4-acryloylpiperazin-1-yl)-2-hydroxyquinazolin-6-yl)-2-methoxypyridin-3-yl)-2,4-difluorobenzenesulfonamide was obtained; yield: 80% z; ¹H NMR (400 MHz, DMSO-d₆) δ 11.07 (s, 1H), 10.30 (s, 1H), 8.32 (d, J=4.0 Hz, 1H), 7.90-7.82 (m, 3H), 7.79-7.73 (m, 1H), 7.58-7.53 (m, 1H), 7.31 (d, J=8.0 Hz, 1H), 7.23-7.18 (m, 1H), 6.83-6.76 (m, 1H), 6.17-6.12 (m, 1H), 5.73-5.70 (m, 1H), 3.82-3.72 (m, 8H), 3.63 (s, 3H); ESI-MS(m/z): 583.0 [M+H]⁺.

### Example 33

### Preparation of N-(5-(4-(4-acryloylpiperazin-1-yl)quinazolin-6-yl)-2-chloropyridin-3-yl)-2,4-difluorobenzenesulfonamide

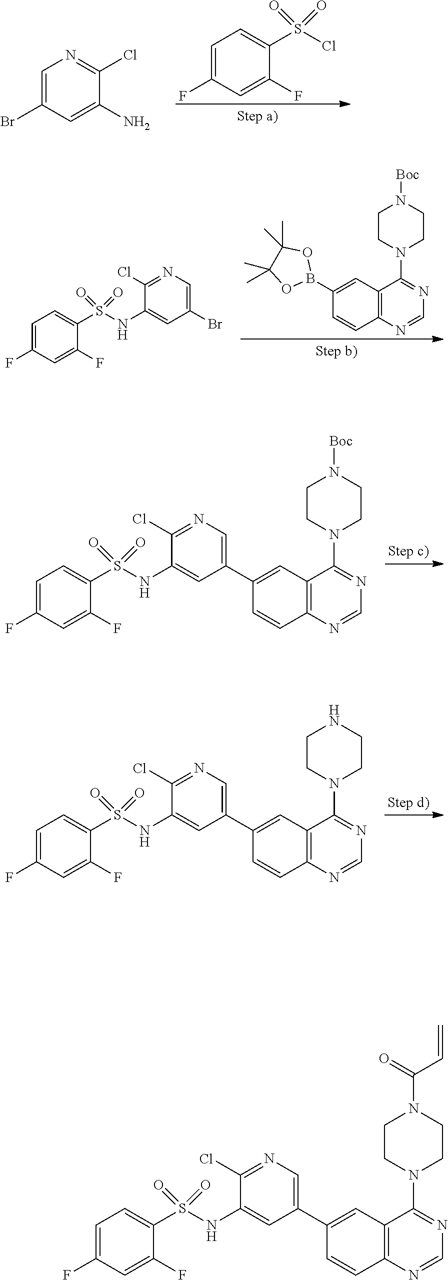

### Step a): Preparation of N-(5-bromo-2-chloropyridin-3-yl)-2,4-difluorobenzenesulfonamide

5-bromo-2-chloropyridin-3-amine (1.5 g, 7.230 mmol), 2,4-difluorobenzenesulfonyl chloride (1.69 g, 7.950 mmol) were dissolved in pyridine (20 mL). After the addition was completed, the reaction mixture was heated to 80° C., stirred and reacted overnight. Upon completion of the reaction, the reaction was quenched by adding water (50 mL). The reaction mixture was extracted with ethyl acetate (200 mL×2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=10/1 to 3/1). N-(5-bromo-2-chloropyridin-3-yl)-2,4-difluorobenzenesulfonamide was obtained. ESC-MS(m/z): 384.9 [M+H]⁺.

### Step b): Preparation of tert-butyl 4-(6-(6-chloro-5-((2,4-difluorophenyl)sulfonamido)pyridin-3-yl)quinazolin-4-yl)piperazine-1-carboxylate

N-(5-bromo-2-chloropyridin-3-yl)-2,4-difluorobenzenesulfonamide (300 mg, 0.782 mmol), tert-butyl 4-(6-(4,4,5,5-tetramethyl-1,3,2-dioxaborolan-2-yl)quinazolin-4-yl)piperazine-1-carboxylate (379 mg, 0.860 mmol), Pd(dppf)Cl₂ (114 mg, 0.156 mmol) and cesium carbonate (764 mg, 2.346 mmol) were added to dioxane/water mixed solvent (10 mL, v/v=10:1). After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (150 mL×2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=5/1 to 1/1). Tert-butyl 4-(6-(6-chloro-5-((2,4-difluorophenyl)sulfonamido)pyridin-3-yl)quinazolin-4-yl)piperazine-1-carboxylate was obtained, yield: 51.8%; ESI-MS(m/z): 617.2 [M+H]⁺.

### Step c): Preparation of N-(2-chloro-5-(4-(piperazin-1-yl)quinazolin-6-yl)pyridin-3-yl)-2,4-difluorobenzenesulfonamide

Tert-butyl 4-(6-(6-chloro-5-((2,4-difluorophenyl)sulfonamido)pyridin-3-yl)quinazolin-4-yl)piperazine-1-carboxylate (100 mg, 0.162 mmol) was dissolved in dichloromethane (5 mL). Under the ice bath condition, trifluoroacetic acid (2 mL) was slowly added dropwise. After the addition was completed, the reaction mixture was stirred at room temperature for 1 h. Upon completion of the reaction, the reaction mixture was concentrated under reduced pressure. N-(2-chloro-5-(4-(piperazin-1-yl)quinazolin-6-yl)pyridin-3-yl)-2,4-difluorobenzenesulfonamide was obtained. The product can be used directly in the next reaction without further purification. ESI-MS(m/z): 517.1 [M+H]⁺.

### Step d): Preparation of N-(5-(4-(4-acryloylpiperazin-1-yl)quinazolin-6-yl)-2-chloropyridin-3-yl)-2,4-difluorobenzenesulfonamide

N-(2-chloro-5-(4-(piperazin-1-yl)quinazolin-6-yl)pyridin-3-yl)-2,4-difluorobenzenesulfonamide (50 mg, 0.097 mmol) was dissolved in tetrahydrofuran (2 mL). The reaction system was cooled to −78° C., with N,N-diisopropylethylamine (63 mg, 0.485 mmol), acrylic acid (4 mg, 0.097 mmol) and 50% T₃P ethyl acetate solution (123 mg, 0.194 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC. N-(5-(4-(4-acryloylpiperazin-1-yl)quinazolin-6-yl)-2-chloropyridin-3-yl)-2,4-difluorobenzenesulfonamide was obtained, yield: 17.6%; ¹H NMR (400 MHz, DMSO-d₆) δ 8.69 (s, 2H), 8.23-8.13 (m, 3H), 7.95-7.93 (m, 1H), 7.85-7.79 (m, 1H), 7.58-7.50 (m, 1H), 7.26-7.19 (m, 1H), 6.86-6.80 (m, 1H), 6.20-6.15 (m, 1H), 5.75-5.73 (m, 1H), 3.98-3.92 (m, 4H), 3.85-3.79 (m, 4H); ESI-MS(m/z): 571.0 [M+H]r.

### Example 34

### Preparation of (E)-N-(2-chloro-5-(4-(4-(4-oxopent-2-enoyl)piperazin-1-yl)quinazolin-6-yl)pyridin-3-yl)-2,4-difluorobenzenesulfonamide

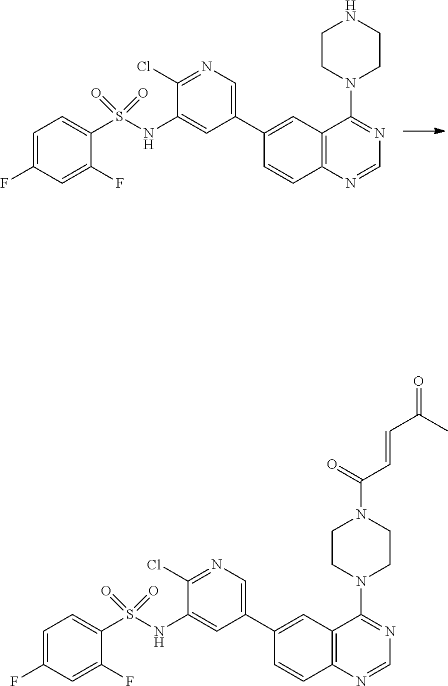 [SMILES: O=S(=O)(Nc1cc(-c2ccc3ncnc(N4CCNCC4)c3c2)cnc1Cl)c1ccc(F)cc1F ; CC(=O)/C=C/C(=O)N1CCN(c2ncnc3ccc(-c4cnc(Cl)c(NS(=O)(=O)c5ccc(F)cc5F)c4)cc23)CC1]

### Step a): Preparation of (E)-N-(2-chloro-5-(4-(4-(4-oxopent-2-enoyl)piperazin-1-yl)quinazolin-6-yl)pyridin-3-yl)-2,4-difluorobenzenesulfonamide

N-(2-chloro-5-(4-(piperazin-1-yl)quinazolin-6-yl)pyridin-3-yl)-2,4-difluorobenzenesulfonamide (50 mg, 0.097 mmol) was dissolved in tetrahydrofuran (2 mL). The reaction system was cooled to −78° C., with N,N-diisopropylethylamine (63 mg, 0.485 mmol), (E)-4-oxopent-2-enoic acid (1 mg, 0.097 mmol) and 50% T₃P ethyl acetate solution (123 mg, 0.194 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC. (E)-N-(2-chloro-5-(4-(4-(4-oxopent-2-enoyl)piperazin-1-yl)quinazolin-6-yl)pyridin-3-yl)-2,4-difluorobenzenesulfonamide was obtained, yield: 16.9%; ¹H NMR (400 MHz, DMSO-d₆) δ 8.68 (s, 1H), 8.31 (s, 1H), 8.12 (s, 1H), 8.04-7.99 (m, 2H), 7.93 (d, J=8.0 MHz, 1H), 7.86-7.80 (m, 1H), 7.45 (d, J=16.0 MHz, 1H), 7.41-7.35 (m, 1H), 7.18-7.13 (m, 2H), 6.74 (d, J=16.0 MHz, 1H), 3.89-3.78 (m, 8H), 2.37 (s, 3H); ESI-MS(m/z): 613.0 [M+H]⁺.

### Example 35

### Preparation of N-(5-(4-(4-acryloylpiperazin-1-yl)quinazolin-6-yl)-2-chloropyridin-3-yl)-5-chlorothiophene-2-sulfonamide

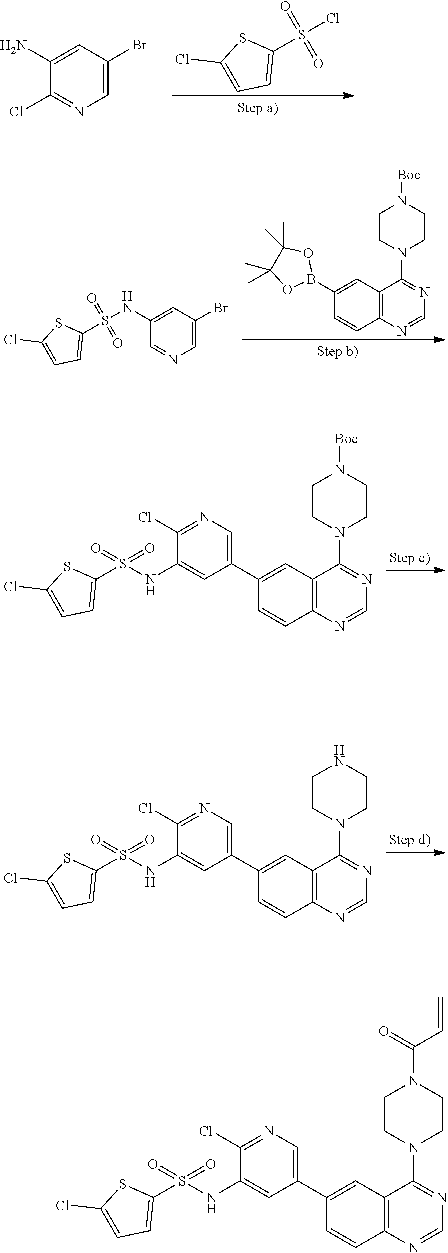

### Step a): Preparation of N-(5-bromopyridin-3-yl)-5-chlorothiophene-2-sulfonamide

5-bromo-2-chloropyridin-3-amine (1.0 g, 4.820 mmol), 5-chlorothiophene-2-sulfonyl chloride (994 mg, 4.579 mmol) were dissolved in pyridine (10 mL). After the addition was completed, at 80° C., the reaction mixture was stirred and reacted overnight. Upon completion of the reaction, the reaction was quenched by adding water (50 mL). The reaction mixture was extracted with ethyl acetate (200 mL×2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=10/1 to 3/1). N-(5-bromopyridin-3-yl)-5-chlorothiophene-2-sulfonamide was obtained, yield: 49.7%. ESI-MS(m/z): 387.8 [M+H]⁺.

### Step b): Preparation of tert-butyl 4-(6-(6-chloro-5-((5-chlorothiophene)-2-sulfonamido)pyridin-3-yl)quinazolin-4-yl)piperazine-1-carboxylate

N-(5-bromopyridin-3-yl)-5-chlorothiophene-2-sulfonamide (500 mg, 1.289 mmol), tert-butyl 4-(6-(4,4,5,5-tetramethyl-1,3,2-dioxaborolan-2-yl)quinazolin-4-yl)piperazine-1-carboxylate (624 mg, 1.418 mmol), Pd(dppf)Cl₂ (189 mg, 0.258 mmol) and cesium carbonate (1.26 g, 3.867 mmol) were added to dioxane/water mixed solvent (10 mL, v/v=10:1). After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (200 mL×2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: dichloromethane/methanol=100/1 to 40/1). Tert-butyl 4-(6-(6-chloro-5-((5-chlorothiophene)-2-sulfonamido)pyridin-3-yl)quinazolin-4-yl)piperazine-1-carboxylate was obtained, yield: 55.2%; ESI-MS(m/z): 621.1 [M+H]⁺.

### Step c): Preparation of 5-chloro-N-(2-chloro-5-(4-(piperazin-1-yl)quinazolin-6-yl)pyridin-3-yl)thiophene-2-sulfonamide

Tert-butyl 4-(6-(6-chloro-5-((5-chlorothiophene)-2-sulfonamido)pyridin-3-yl)quinazolin-4-yl)piperazine-1-carboxylate (500 mg, 0.804 mmol) was dissolved in dichloromethane (5 mL). Under the ice bath condition, trifluoroacetic acid (2 mL) was slowly added dropwise. After the addition was completed, the reaction mixture was stirred at room temperature for 1 h. Upon completion of the reaction, the reaction mixture was concentrated under reduced pressure. 5-chloro-N-(2-chloro-5-(4-(piperazin-1-yl)quinazolin-6-yl)pyridin-3-yl)thiophene-2-sulfonamide was obtained. The product can be used directly in the next reaction without further purification. ESI-MS(m/z): 521.0 [M+H]⁺.

### Step d): Preparation of N-(5-(4-(4-acryloylpiperazin-1-yl)quinazolin-6-yl)-2-chloropyridin-3-yl)-5-chlorothiophene-2-sulfonamide

5-chloro-N-(2-chloro-5-(4-(piperazin-1-yl)quinazolin-6-yl)pyridin-3-yl)thiophene-2-sulfonamide (100 mg, 0.192 mmol) was dissolved in tetrahydrofuran (2 mL). The reaction system was cooled to −78 C. with N,N-diisopropylethylamine (124 mg, 0.960 mmol), acrylic acid (14 mg, 0.192 mmol) and 50% T₃P ethyl acetate solution (244 mg, 0.384 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC. N-(5-(4-(4-acryloylpiperazin-1-yl)quinazolin-6-yl)-2-chloropyridin-3-yl)-5-chlorothiophene-2-sulfonamide was obtained, yield: 15.3%. ¹H NMR (400 MHz, DMSO-d₆) δ 8.70 (s, 1H), 8.63-8.62 (m, 1H), 8.23-8.22 (m, 1H), 8.15-8.14 (m, 1H), 8.13-8.12 (m, 1H), 7.96-7.93 (m, 1H), 7.39-7.37 (m, 1H), 7.21-7.20 (m, 1H), 6.87-6.80 (m, 1H), 6.20-6.15 (m, 4H), 5.76-5.73 (m, 4H), 3.97-3.79 (m, 8H); ESI-MS(m/z): 575.0 [M+H]⁺.

### Example 36

### Preparation of (E)-5-chloro-N-(2-chloro-5-(4-(4-(4-oxopent-2-enoyl)piperazin-1-yl)quinazolin-6-yl)pyridin-3-yl)thiophene-2-sulfonamide

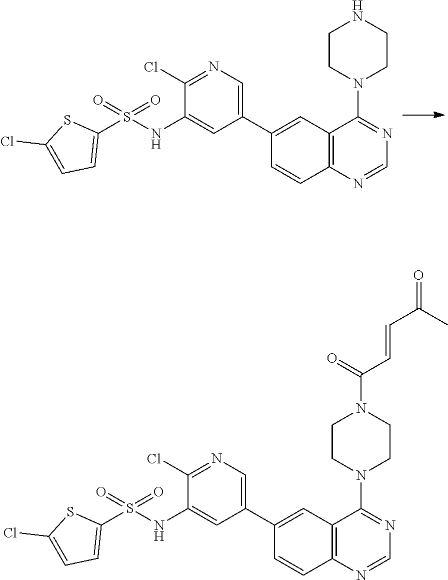 [SMILES: O=S(=O)(Nc1cc(-c2ccc3ncnc(N4CCNCC4)c3c2)cnc1Cl)c1ccc(Cl)s1 ; CC(=O)/C=C/C(=O)N1CCN(c2ncnc3ccc(-c4cnc(Cl)c(NS(=O)(=O)c5ccc(Cl)s5)c4)cc23)CC1]

5-chloro-N-(2-chloro-5-(4-(piperazin-1-yl)quinazolin-6-yl)pyridin-3-yl)thiophene-2-sulfonamide (100 mg, 0.192 mmol) was dissolved in tetrahydrofuran (2 mL). The reaction system was cooled to −78° C. with N,N-diisopropylethylamine (124 mg, 0.960 mmol), (E)-4-oxopent-2-enoic acid (22 mg, 0.192 mmol) and 50% T₃P ethyl acetate solution (244 mg, 0.384 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC. (E)-5-chloro-N-(2-chloro-5-(4-(4-(4-oxopent-2-enoyl)piperazin-1-yl)quinazolin-6-yl)pyridin-3-yl)thiophene-2-sulfonamide was obtained, yield: 14.4%; ¹H NMR (400 MHz, DMSO-d₆) δ 8.71 (s, 1H), 8.61-8.60 (m, 1H), 8.23-8.22 (m, 1H), 8.15-8.14 (m, 1H), 8.13-8.11 (m, 1H), 7.95 (d, J=8.0 MHz, 1H), 7.47-7.37 (m, 2H), 7.21-7.20 (m, 1H), 6.75 (d, J=16.0 MHz, 1H), 3.98-3.80 (m, 8H), 2.37 (s, 3H); ESI-MS(m/z): 617.0 [M+H]⁺.

### Example 37

### Preparation of N-(5-(4-(4-acryloylpiperazin-1-yl)quinazolin-6-yl)-2-methoxypyridin-3-yl)-5-chlorothiophene-2-sulfonamide

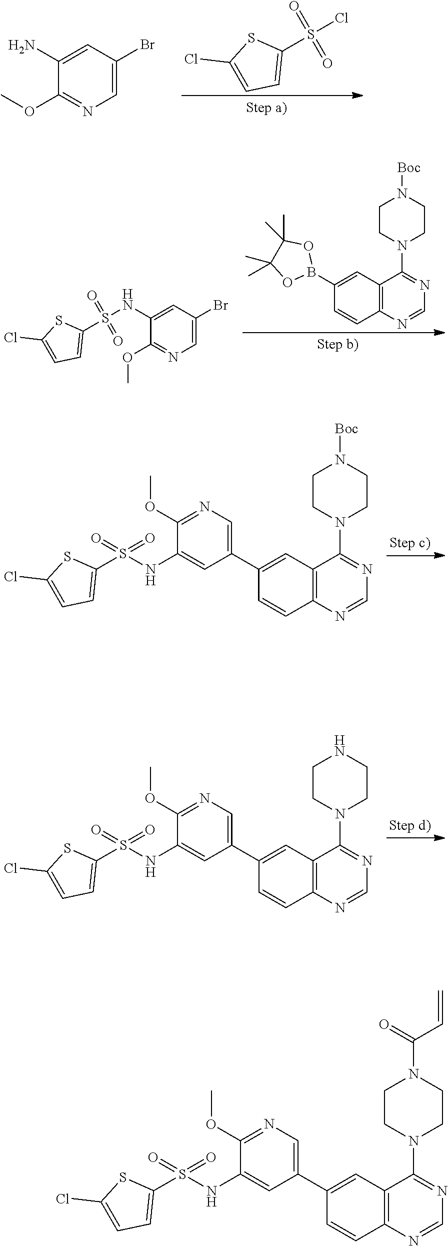

### Step a): Preparation of N-(5-bromo-2-methoxypyridin-3-yl)-5-chlorothiophene-2-sulfonamide

5-bromo-2-methoxypyridin-3-amine (1.0 g, 4.925 mmol), 5-chlorothiophene-2-sulfonyl chloride (1.02 g, 4.679 mmol) were dissolved in pyridine (10 mL). After the addition was completed, at 80° C., the reaction mixture was stirred and reacted overnight. Upon completion of the reaction, the reaction was quenched by adding water (50 mL). The reaction mixture was extracted with ethyl acetate (200 mL×2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=10/1 to 3/1). N-(5-bromo-2-methoxypyridin-3-yl)-5-chlorothiophene-2-sulfonamide was obtained, yield: 78.5%. ESI-MS(m/z): 384.9 [M+H]⁺.

### Step b): Preparation of tert-butyl 4-(6-(5-((5-chlorothiophene)-2-sulfonamido)-6-methoxypyridin-3-yl)quinazolin-4-yl)piperazine-1-carboxylate

N-(5-bromo-2-methoxypyridin-3-yl)-5-chlorothiophene-2-sulfonamide (300 mg, 0.782 mmol), tert-butyl 4-(6-(4,4,5,5-tetramethyl-1,3,2-dioxaborolan-2-yl)quinazolin-4-yl)piperazine-1-carboxylate (370 mg, 0.782 mmol), Pd(dppf)Cl₂ (114 mg, 0.156 mmol) and cesium carbonate (750 mg, 2.346 mmol) were added to dioxane/water mixed solvent (10 mL, v/v=10:1). After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (200 mL×2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: dichloromethane/methanol=100/1 to 40/1). Tert-butyl 4-(6-(5-((5-chlorothiophene)-2-sulfonamido)-6-methoxypyridin-3-yl)quinazolin-4-yl)piperazine-1-carboxylate was obtained, yield: 50.7%; ESI-MS(m/z): 617.1 [M+H]⁺.

### Step c): Preparation of 5-chloro-N-(2-methoxy-5-(4-(piperazin-1-yl)quinazolin-6-yl)pyridin-3-yl)thiophene-2-sulfonamide

Tert-butyl 4-(6-(5-((5-chlorothiophene)-2-sulfonamido)-6-methoxypyridin-3-yl)quinazolin-4-yl)piperazine-1-carboxylate (200 mg, 0.804 mmol) was dissolved in dichloromethane (3 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. 5-chloro-N-(2-methoxy-5-(4-(piperazin-1-yl)quinazolin-6-yl)pyridin-3-yl)thiophene-2-sulfonamide was obtained. ESI-MS(m/z): 517.1 [M+H]⁺.

### Step d): Preparation of N-(5-(4-(4-acryloylpiperazin-1-yl)quinazolin-6-yl)-2-methoxypyridin-3-yl)-5-chlorothiophene-2-sulfonamide

5-chloro-N-(2-methoxy-5-(4-(piperazin-1-yl)quinazolin-6-yl)pyridin-3-yl)thiophene-2-sulfonamide (100 mg, 0.193 mmol) was dissolved in tetrahydrofuran (2 mL). The reaction system was cooled to −78° C. with N,N-diisopropylethylamine (124 mg, 0.960 mmol), acrylic acid (14 mg, 0.193 mmol) and 50% T₃P ethyl acetate solution (245 mg, 0.386 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC. N-(5-(4-(4-acryloylpiperazin-1-yl)quinazolin-6-yl)-2-methoxypyridin-3-yl)-5-chlorothiophene-2-sulfonamide was obtained, yield: 16.6%; ¹H NMR (400 MHz, DMSO-d₆) δ 8.66 (s, 1H), 8.51-8.50 (m, 1H), 8.15-8.09 (m, 1H), 8.02-8.01 (m, 1H), 7.93-7.91 (m, 1H), 7.42-7.41 (m, 1H), 7.23-7.21 (m, 1H), 6.86-6.80 (m, 1H), 6.19-6.14 (m, 4H), 5.75-5.72 (m, 1H), 3.91-3.79 (m, 8H), 3.78 (s, 3H); ESI-MS(m/z): 571.0 [M+H]⁺.

### Example 38

### Preparation of (E)-5-chloro-N-(2-methoxy-5-(4-(4-(4-oxopent-2-enoyl)piperazin-1-yl)quinazolin-6-yl)pyridin-3-yl)thiophene-2-sulfonamide

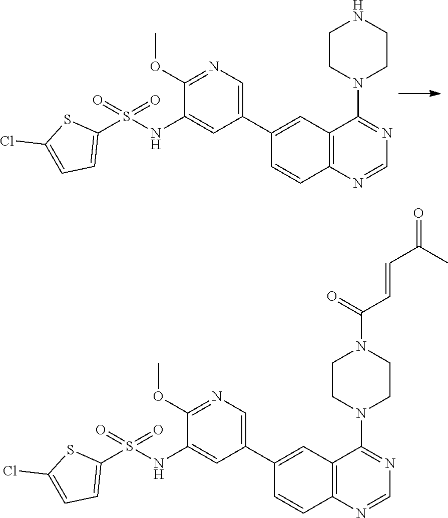 [SMILES: COc1ncc(-c2ccc3ncnc(N4CCNCC4)c3c2)cc1NS(=O)(=O)c1ccc(Cl)s1 ; COc1ncc(-c2ccc3ncnc(N4CCN(C(=O)/C=C/C(C)=O)CC4)c3c2)cc1NS(=O)(=O)c1ccc(Cl)s1]

5-chloro-N-(2-methoxy-5-(4-(piperazin-1-yl)quinazolin-6-yl)pyridin-3-yl)thiophene-2-sulfonamide (100 mg, 0.193 mmol) was added to tetrahydrofuran (2 mL). The reaction system was stirred to mix evenly and cooled to −78° C. with N,N-diisopropylethylamine (124 mg, 0.960 mmol), (E)-4-oxopent-2-enoic acid (22 mg, 0.193 mmol) and 50% T₃P ethyl acetate solution (245 mg, 0.386 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC. (E)-5-chloro-N-(2-methoxy-5-(4-(4-(4-oxopent-2-enoyl)piperazin-1-yl)quinazolin-6-yl)pyridin-3-yl)thiophene-2-sulfonamide was obtained, yield: 18.1%; ¹H NMR (400 MHz, DMSO-d₆) δ 10.42 (s, 1H), 8.69 (s, 1H), 8.53-8.52 (m, 1H), 8.17-8.11 (m, 2H), 8.03 (d, J=4.0 Hz, 1H), 7.93 (d, J=8.0 Hz, 1H), 7.47-7.41 (m, 2H), 7.24 (d, J=4.0 Hz, 1H), 6.77-6.73 (d, J=16.0 Hz, 1H), 3.96-3.80 (m, 8H), 3.78 (s, 3H), 2.37 (s, 3H); ESI-MS(m/z): 613.0 [M+H]⁺.

### Example 39

### Preparation of (E)-2,4-difluoro-N-(1-methyl-2-oxo-5-(4-(4-(4-oxopent-2-enoyl)piperazin-1-yl)quinazolin-6-yl)-1,2-dihydropyridin-3-yl)benzenesulfonamide

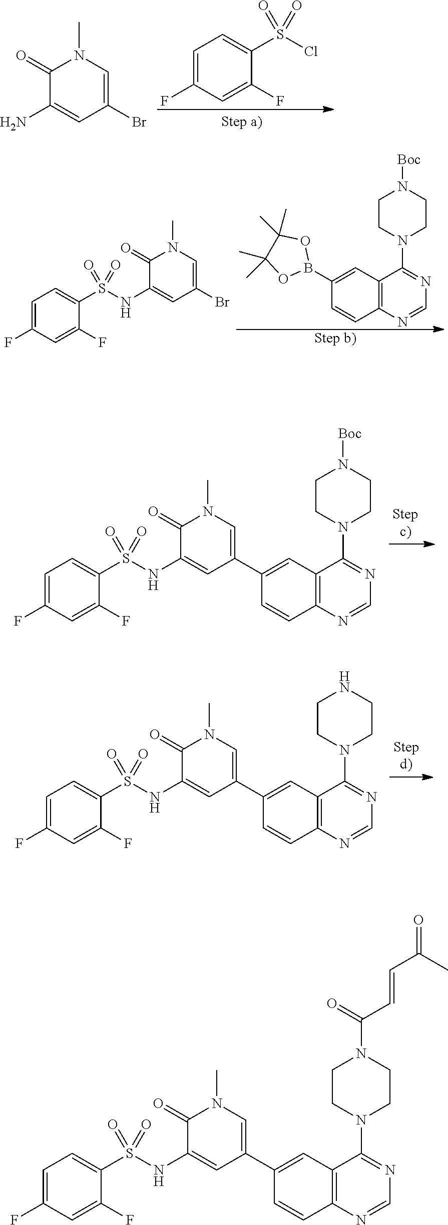

### Step a): Preparation of N-(5-bromo-1-methyl-2-oxo-1,2-dihydropyridin-3-yl)-2,4-difluorobenznesulfonamide

3-amino-5-bromo-1-methylpyridin-2 (1H)-one (500 mg, 2.46 mmol), 4-dimethylaminopyridine (30 mg, 0.25 mmol) and pyridine (600 mg, 7.38 mmol) were dissolved in dichloromethane (15.0 mL), the reaction mixture was cooled to 0° C. with 2,4-difluorobenzenesulfonyl chloride (1.57 g, 7.38 mmol) added. The reaction mixture was heated to 25° C. and stirred for 4 h. Upon completion of the reaction, the reaction mixture was diluted by adding dichloromethane (20 mL). The organic phase was washed with water (15 mL 1), 5% citric acid aqueous solution (30 mL×1), and saturated brine (15 mL×1) successively, concentrated under reduced pressure. With acetonitrile (8 mL) added, the crude product was kept at 45° C. and stirred for 1 h, then kept at 25° C. and stirred for 20 h. The solution was filtered after solid precipitation appeared. N-(5-bromo-1-methyl-2-oxo-1,2-dihydropyridin-3-yl)-2,4-difluorobenzenesulfonamide was obtained, yield: 70.0%; ESI-MS(m/z): 378.9 [M+H]⁺.

### Step b): Preparation of tert-butyl 4-(6-(5-((2,4-difluorophenyl)sulfonamido)-1-methyl-6-oxo-1,6-dihydropyridin-3-yl)quinazolin-4-yl)piperazine-1-carboxylate

N-(5-bromo-1-methyl-2-oxo-1,2-dihydropyridin-3-yl)-2,4-difluorobenzenesulfonamide (156 mg, 0.413 mmol), tert-butyl 4-(6-(4,4,5,5-tetramethyl-1,3,2-dioxaborolan-2-yl)quinazolin-4-yl)piperazine-1-carboxylate (200 mg, 0.454 mmol), Pd(dppf)Cl₂ (45 mg, 0.06 mmol) and cesium carbonate (402 mg, 1.24 mmol) were added to dioxane/water mixed solvent (10 mL, v/v=4:1). After the addition was completed, in the presense of protective nitrogen, the reaction mixture was heated to 95° C. and stirred for 0.5 h. Upon completion of the reaction, the reaction mixture was cooled to 25° C., with reaction quenched by adding water (15 mL), extracted with ethyl acetate (20 mL×2). The organic phases were combined, dried over anhydrous sodium sulfate and filtered. The filtrate was concentrated under reduced pressure. The crude product obtained was purified by silica gel column chromatography (Eluent: dichloromethane/methanol=100/1 to 50/1). Tert-butyl 4-(6-(5-((2,4-difluorophenyl)sulfonamido)-1-methyl-6-oxo-1,6-dihydropyridin-3-yl)quinazolin-4-yl)piperazine-1-carboxylate was obtained, yield: 31.7%; ESI-MS(m/z): 613.2 [M+H]⁺.

### Step c): Preparation of 2,4-difluoro-N-(1-methyl-2-oxo-5-(4-(piperazin-1-yl)quinazolin-6-yl)-1,2-dihydropyridin-3-yl)benzenesulfonamide trifluoroacetate

Tert-butyl 4-(6-(5-((2,4-difluorophenyl)sulfonamido)-1-methyl-6-oxo-1,6-dihydropyridin-3-yl)quinazolin-4-yl)piperazine-1-carboxylate (80 mg, 13 mmol) was dissolved in dichloromethane (2.5 mL). Under the ice bath condition, trifluoroacetic acid (0.5 mL) was slowly added dropwise. After the addition was completed, the reaction mixture was stirred at room temperature for 3 h. Upon completion of the reaction, the reaction mixture was concentrated under reduced pressure. Oily crude product was obtained, with methyl tert-butyl ether (5 mL) added, and stirred for 30 min. The solution was filtered after solid precipitation appeared, 2,4-difluoro-N-(1-methyl-2-oxo-5-(4-(piperazin-1-yl)quinazolin-6-yl)-1,2-dihydropyridin-3-yl)benzenesulfonamide trifluoroacetate was obtained, yield: 73.7%. ESI-MS(m/z): 513.14 [M+H]⁺.

### Step d): Preparation of (E)-2,4-difluoro-N-(1-methyl-2-oxo-5-(4-(4-(4-oxopent-2-enoyl)piperazin-1-yl)quinazolin-6-yl)-1,2-dihydropyridin-3-yl)benzenesulfonamide

2,4-difluoro-N-(1-methyl-2-oxo-5-(4-(piperazin-1-yl)quinazolin-6-yl)-1,2-dihydropyridin-3-yl)benzenesulfonamide trifluoroacetate (60 mg, 0.096 mmol) was added to tetrahydrofuran (2 mL). The reaction system was cooled to −70° C., with N,N-diisopropylethylamine (75 mg, 0.575 mmol), 3-acetylacrylic acid (11 mg, 0.096 mmol) and T₃P (122 mg, 0.193 mmol) added successively. After the addition was completed, the reaction mixture was kept at −70° C. and stirred for 0.5 h. Upon completion of the reaction, the reaction mixture was filtered and purified by Prep-HPLC. (E)-2,4-difluoro-N-(1-methyl-2-oxo-5-(4-(4-(4-oxopent-2-enoyl)piperazin-1-yl)quinazolin-6-yl)-1,2-dihydropyridin-3-yl)benzenesulfonamide was obtained, yield 39.6%; ¹H NMR (400 MHz, DMSO-4) δ 9.86 (brs, 1H), 8.66 (s, 1H), 8.11 (s, 1H), 8.01-7.98 (m, 2H), 7.96-7.92 (m, 1H), 7.91-7.85 (m, 2H), 7.54-7.49 (m, 1H), 7.46 (d, J=16 Hz, 1H), 7.27-7.22 (m, 1H), 6.76 (d, J=16.0 Hz, 1H), 3.87-3.82 (m, 8H), 3.53 (s, 3H), 2.36 (s, 3H); ESI-MS(m/z): 609.0 [M+H]⁺.

### Example 40

### Preparation of N-(5-(4-(4-acryloylpiperazin-1-yl)quinazolin-7-yl)-2-methoxypyridin-3-yl)-2,4-difluorobenzenesulfonamide

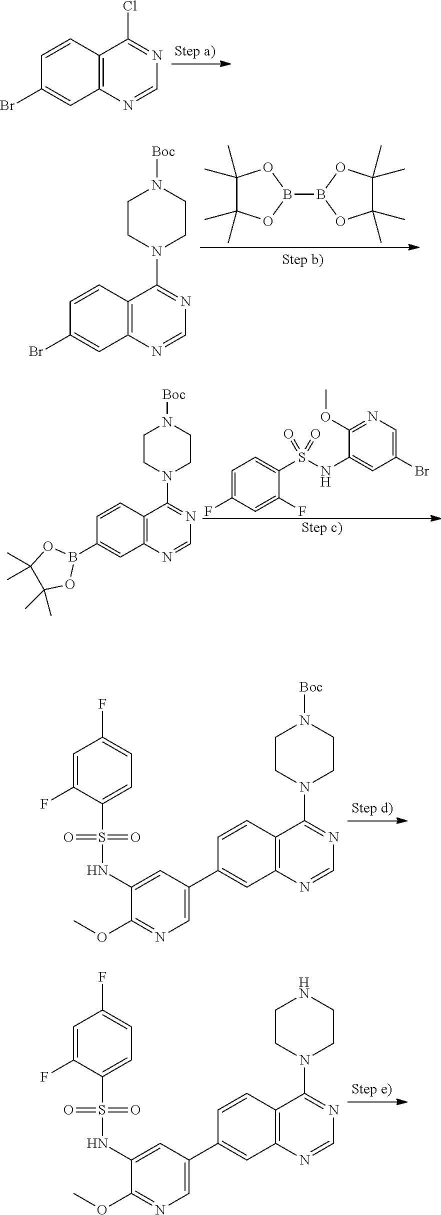

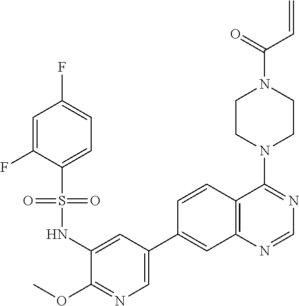 [SMILES: C=CC(=O)N1CCN(c2ncnc3cc(-c4cnc(OC)c(NS(=O)(=O)c5ccc(F)cc5F)c4)ccc23)CC1]

### Step a): Preparation of tert-butyl 4-(7-bromoquinazolin-4-yl)piperazine-1-carboxylate

7-Bromo-4-chloroquinazoline (500 mg, 2.053 mmol) and tert-butyl piperazine-1-carboxylate (420 mg, 2.255 mmol) were dissolved in dimethyl sulfoxide (10 mL), with N,N-diisopropylethylamine (796 mg, 6.159 mmol) added. After the addition was completed, the reaction mixture was heated to 55° C. and stirred for 1 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=10/1 to 3/1). Tert-butyl 4-(7-bromoquinazolin-4-yl)piperazine-1-carboxylate was obtained, yield: 82.6%; ESI-MS(m/z): 393.1 [M+H]⁺.

### Step b): Preparation of tert-butyl 4-(7-(4,4,5,5-tetramethyl-1,3,2-dioxaborolan-2-yl)quinazolin-4-yl)piperazine-1-carboxylate

Tert-butyl 4-(7-bromoquinazolin-4-yl)piperazine-1-carboxylate (800 mg, 2.034 mmol), bis(pinacolato)diboron (775 mg, 3.051 mmol), Pd(dppf)Cl₂ (149 mg, 0.203 mmol) and potassium acetate (599 mg, 6.102 mmol) were dissolved in dioxane (20 mL). After the addition was completed, in the presense of protective nitrogen, the reaction mixture was kept at 110° C. and stirred for 4 h. Upon completion of the reaction, the reaction mixture was filtered, and the filtrate was concentrated through pressure concentration. The crude product obtained was purified by silica gel column chromatography (Eluent: dichloromethane/methanol=50/1 to 10/1). Tert-butyl 4-(7-(4,4,5,5-tetramethyl-1,3,2-dioxaborolan-2-yl)quinazolin-4-yl)piperazine-1-carboxylate was obtained, yield: 66.6%; ESI-MS(m/z): 441.3 [M+H]⁺.

### Step c): Preparation of tert-butyl 4-(7-(5-((2,4-difluorophenyl)sulfonamido)-6-methoxypyridin-3-yl)quinazolin-4-yl)piperazine-1-carboxylate

Tert-butyl 4-(7-(4,4,5,5-tetramethyl-1,3,2-dioxaborolan-2-yl)quinazolin-4-yl)piperazine-1-carboxylate (735 mg, 1.668 mmol), N-(5-bromo-2-methoxypyridin-3-yl)-2,4-difluorobenzenesulfonamide (500 mg, 1.319 mmol), Pd(dppf)Cl₂ (96 mg, 0.132 mmol) and cesium carbonate (1.289 g, 3.957 mmol) were added to dioxane/water mixed solvent (25 mL, v/v=10:1). After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (150 mL×2 times). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=2/1 to 1/1). Tert-butyl 4-(7-(5-((2,4-difluorophenyl)sulfonamido)-6-methoxypyridin-3-yl)quinazolin-4-yl)piperazine-1-carboxylate was obtained, yield: 37.8%; ESI-MS(m/z): 613.2 [M+H]⁺.

### Step d): Preparation of 2,4-difluoro-N-(2-methoxy-5-(4-(piperazin-1-yl)quinazolin-7-yl)pyridin-3-yl)benzenesulfonamide

Tert-butyl 4-(7-(5-((2,4-difluorophenyl)sulfonamido)-6-methoxypyridin-3-yl)quinazolin-4-yl)piperazine-1-carboxylate (200 mg, 0.326 mmol) was dissolved in dichloromethane (4 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. 2,4-difluoro-N-(2-methoxy-5-(4-(piperazin-1-yl)quinazolin-7-yl)pyridin-3-yl)benzenesulfonamide was obtained. ESI-MS(m/z): 513.1 [M+H]⁺.

### Step e): Preparation of N-(5-(4-(4-acryloylpiperazin-1-yl)quinazolin-7-yl)-2-methoxypyridin-3-yl)-2,4-difluorobenzenesulfonamide

2,4-difluoro-N-(2-methoxy-5-(4-(piperazin-1-yl)quinazolin-7-yl)pyridin-3-yl)benzenesulfonamide (100 mg, 0.195 mmol) was dissolved in tetrahydrofuran (3 mL). The reaction system was cooled to −78° C., with N,N-diisopropylethylamine (126 mg, 0.975 mmol), acrylic acid (14.0 mg, 0.195 mmol) and 50% T₃P ethyl acetate solution (124 mg, 0.390 mmol) added successively. After the addition was completed, the reaction mixture was kept at −78° C. and stirred for 1 h. Upon completion of the reaction, the reaction was quenched by adding acetonitrile (2 mL), the reaction solution was purified by Prep-HPLC. N-(5-(4-(4-acryloylpiperazin-1-yl)quinazolin-7-yl)-2-methoxypyridin-3-yl)-2,4-difluorobenzenesulfonamide was obtained, yield: 13.1%; ¹H NMR (400 MHz, DMSO-d₆) δ 10.37 (brs, 1H), 8.67 (s, 1H), 8.51 (s, 1H), 8.24 (s, 1H), 8.16-8.13 (m, 1H), 8.04-8.02 (m, 1H), 7.84-7.76 (m, 2H), 7.60-7.54 (m, 1H), 7.24-7.19 (m, 1H), 6.89-6.82 (m, 1H), 6.20-6.15 (m, 1H), 5.76-5.73 (m, 1H), 3.84-3.77 (m, 8H), 3.70 (s, 3H); ESI-MS(m/z): 567.0 [M+H]⁺.

### Example 41

### Preparation of N-(5-(4-(4-acryloylpiperazin-1-yl)-7,8-dihydropyrido[4,3-d]pyrimidin-6 (5H)-yl)-2-methoxypyridin-3-yl)-2,4-difluorobenzenesulfonamide

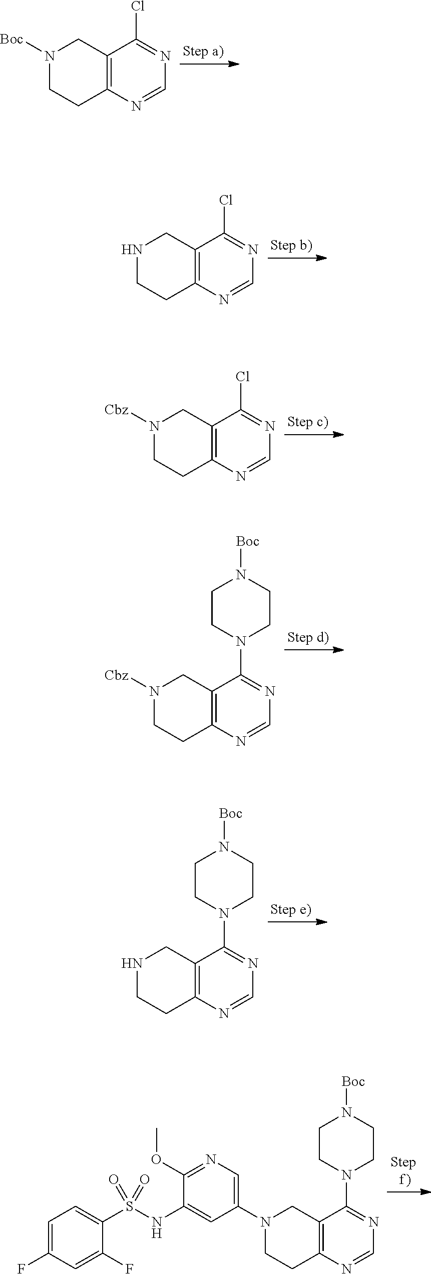 [SMILES: CC(C)(C)OC(=O)N1CCc2ncnc(Cl)c2C1 ; Clc1ncnc2c1CNCC2 ; O=C(OCc1ccccc1)N1CCc2ncnc(Cl)c2C1 ; CC(C)(C)OC(=O)N1CCN(c2ncnc3c2CN(C(=O)OCc2ccccc2)CC3)CC1 ; CC(C)(C)OC(=O)N1CCN(c2ncnc3c2CNCC3)CC1 ; COc1ncc(N2CCc3ncnc(N4CCN(C(=O)OC(C)(C)C)CC4)c3C2)cc1NS(=O)(=O)c1ccc(F)cc1F]

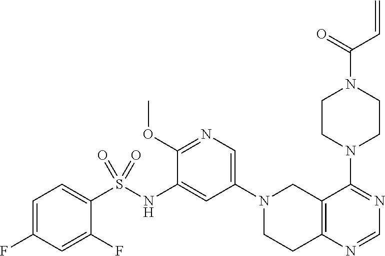 [SMILES: C=CC(=O)N1CCN(c2ncnc3c2CN(c2cnc(OC)c(NS(=O)(=O)c4ccc(F)cc4F)c2)CC3)CC1]

### Step a): Preparation of 4-chloro-5,6,7,8-tetrahydropyrido[4,3-d]pyrimidine

Tert-butyl 4-chloro-7,8-dihydropyrido[4,3-d]pyrimidine-6 (5H)-carboxylate (3.0 g, 11.111 mmol) was dissolved in dichloromethane (10 mL). Under the ice bath condition, HCl dioxane solution (4M, 10 mL) was added to the system. After the addition was completed, the reaction mixture was heated to room temperature and stirred for 1 h. Upon completion of the reaction, the reaction mixture was concentrated under reduced pressure. 4-chloro-5,6,7,8-tetrahydropyrido[4,3-d]pyrimidine was obtained, yield: 90.3%; ESI-MS(m/z): 170.1 [M+H]⁺.

### Step b): Preparation of benzyl 4-chloro-7,8-dihydropyrido[4,3-d]pyrimidine-6 (5H)-carboxylate

4-chloro-5,6,7,8-tetrahydropyrido[4,3-d]pyrimidine (1.7 g, 1.003 mmol) was dissolved in dichloromethane (30 mL). Under the ice bath condition, triethylamine (5.1 g, 5.015 mmol) and benzyl chloroformate (2.6 g, 1.505 mmol) were added to the system. After the addition was completed, the reaction mixture was heated to room temperature and reacted for 2 h. Upon completion of the reaction, the reaction was quenched by adding water (50 mL). The reaction mixture was extracted by adding dichloromethane (100 mL×2). The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=3/1 to 1/1). Benzyl 4-chloro-7,8-dihydropyrido[4,3-d]pyrimidine-6 (5H)-carboxylate was obtained, yield: 98.6%; ESI-MS(m/z): 304.3 [M+H]⁺.

### Step c): Preparation of benzyl 4-(4-(tert-butoxycarbonyl)piperazin-1-yl)-7,8-dihydropyrido[4,3-d]pyrimidine-6 (5H)-carboxylate

Benzyl 4-chloro-7,8-dihydropyrido[4,3-d]pyrimidine-6 (5H)-carboxylate (2.9 g, 9.539 mmol) were dissolved in N-methylpyrrolidone (30 mL), with triethylamine (2.9 g, 28.617 mmol) and tert-butyl piperazine-1-carboxylate (2.7 g, 14.309 mmol) added. After the addition was completed, the reaction mixture was heated to 130° C. and stirred for 3 h. Upon completion of the reaction, the reaction was quenched by adding water (50 mL). The reaction mixture was extracted with ethyl acetate (100 mL/2). The organic phases were combined, washed with saturated brine (100 mL 1), dried over anhydrous sodium sulfate and filtered. The filtrate was concentrated under reduced pressure. The crude product obtained was purified by silica gel column chromatography (Eluent: petroleum ether/ethyl acetate=2/1 to 1/1). Benzyl 4-(4-(tert-butoxycarbonyl)piperazin-1-yl)-7,8-dihydropyrido[4,3-d]pyrimidine-6 (5H)-carboxylate was obtained, yield: 92.4%; ESI-MS(m/z): 454.1 [M+H]⁺.

### Step d): Preparation of tert-butyl 4-(5,6,7,8-tetrahydropyrido[4,3-d]pyrimidin-4-yl)piperazine-1-carboxylate

Benzyl 4-(4-(tert-butoxycarbonyl)piperazin-1-yl)-7,8-dihydropyrido[4,3-d]pyrimidine-6 (5H)-carboxylate (500 mg, 1.101 mmol) were dissolved in isopropanol (10 mL), with Pd/C (500 mg, 10%) added. After the addition was completed, the reaction mixture was kept at room temperature and stirred at for 3 h. Upon completion of the reaction, the reaction mixture was filtered. The filtrate was concentrated under reduced pressure. The crude product obtained was purified by silica gel column chromatography (Eluent: petroleum ether/ethyl acetate=2/1 to 1/1). Tert-butyl 4-(5,6,7,8-tetrahydropyrido[4,3-d]pyrimidin-4-yl)piperazine-1-carboxylate was obtained, yield: 85.5%; ESI-MS(m/z): 320.1 [M+H]⁺.

### Step e): Preparation of tert-butyl 4-(6-(5-((2,4-difluorophenyl)sulfonamido)-6-methoxypyridin-3-yl)-5,6,7,8-tetrahydropyrido[4,3-d]pyrimidin-4-yl)piperazine-1-carboxylate

Tert-butyl 4-(5,6,7,8-tetrahydropyrido[4,3-d]pyrimidin-4-yl)piperazine-1-carboxylate (300 mg, 0.940 mmol), N-(5-bromo-2-methoxypyridin-3-yl)-2,4-difluorobenzenesulfonamide (427.7 mg, 1.128 mmol), Pd₂(dba)₃ (172.3 mg, 0.188 mmol), RuPhos (131.8 mg, 0.282 mmol) and cesium carbonate (919.7 mg, 2.821 mmol) were added to toluene (10 mL). After the addition was completed, in the presense of protective nitrogen, the reaction mixture was heated to 110° C. and stirred for 2 h. Upon completion of the reaction, the reaction was quenched by adding water (50 mL). The reaction mixture was extracted with ethyl acetate (100 mL×2) The organic phases were combined, washed with saturated brine (100 mL×1), dried over anhydrous sodium sulfate and filtered. The filtrate was concentrated under reduced pressure. The crude product obtained was purified by silica gel column chromatography (Eluent: petroleum ether/ethyl acetate=1/1 to 1/2). Tert-butyl 4-(6-(5-((2,4-difluorophenyl)sulfonamido)-6-methoxypyridin-3-yl)-5,6,7,8-tetrahydropyrido[4,3-d]pyrimidin-4-yl)piperazine-1-carboxylate was obtained, yield: 34.4%; ESI-MS(m/z): 618.3 [M+1]⁺.

### Step f): Preparation of 2,4-difluoro-N-(2-methoxy-5-(4-(piperazin-1-yl)-7,8-dihydropyrido[4,3-d]pyrimidin-6 (5H)-yl)pyridin-3-yl)benzenesulfonamide

Tert-butyl 4-(6-(5-((2,4-difluorophenyl)sulfonamido)-6-methoxypyridin-3-yl)-5,6,7,8-tetrahydropyrido[4,3-d]pyrimidin-4-yl)piperazine-1-carboxylate (200 mg, 0.324 mmol) was dissolved in dichloromethane (4 mL). Under the ice bath condition, trifluoroacetic acid (2 mL) was slowly added dropwise. After the addition was completed, the reaction mixture was stirred at room temperature for 1 h. Upon completion of the reaction, the reaction mixture was concentrated under reduced pressure. 2,4-difluoro-N-(2-methoxy-5-(4-(piperazin-1-yl)-7,8-dihydropyrido[4,3-d]pyrimidin-6 (5H)-yl)pyridin-3-yl)benzenesulfonamide was obtained. ESI-MS(m/z): 518.2 [M+H]⁺.

### Step g): Preparation of N-(5-(4-(4-acryloylpiperazin-1-yl)-7,8-dihydropyrido[4,3-d]pyrimidin-6 (5H)-yl)-2-methoxypyridin-3-yl)-2,4-difluorobenzenesulfonamide

2,4-difluoro-N-(2-methoxy-5-(4-(piperazin-1-yl)-7,8-dihydropyrido[4,3-d]pyrimidin-6 (5H)-yl)pyridin-3-yl)benzenesulfonamide (150.0 mg, 0.289 mmol) was dissolved in dichloromethane (10 mL). The reaction system was cooled to −78° C., with N,N-diisopropylethylamine (188.2 mg, 1.445 mmol), acrylic acid (31.3 mg, 0.434 mmol) and HATU (165.1 mg, 0.434 mmol) added successively. After the addition was completed, the reaction mixture was kept at −78° C. and stirred for 1 h. Upon completion of the reaction, the reaction was quenched by adding water (5 mL). The reaction mixture was extracted by adding dichloromethane (10 mL/2). The organic phases were combined, dried over anhydrous sodium sulfate and filtered. The filtrate was concentrated under reduced pressure. The crude product obtained was purified by Prep-HPLC. N-(5-(4-(4-acryloylpiperazin-1-yl)-7,8-dihydropyrido[4,3-d]pyrimidin-6 (5H)-yl)-2-methoxypyridin-3-yl)-2,4-difluorobenzenesulfonamide was obtained, yield: 13.1%; ¹H NMR (400 MHz, DMSO-d₆) δ 8.48 (s, 1H), 7.75-7.73 (m, 1H), 7.25-7.20 (m, 1H), 7.10 (s, 2H), 6.88-6.80 (m, 3H), 6.15 (d, J=16.0 Hz, 1H), 5.72 (d, J=16.0 Hz, 1H), 4.01 (s, 2H), 3.70-3.64 (m, 7H), 3.40-3.36 (m, 6H), 2.77-2.74 (m, 2H); ESI-MS(m/z): 572.0 [M+H]⁺.

### Example 42

### Preparation of (E)-2,6-difluoro-N-(2-methoxy-5-(4-(2-(4-oxopent-2-enoyl))-2,6-diazaspiro[3.4]octane-6-yl)quinazolin-6-yl)pyridin-3-yl)benzenesulfonamide

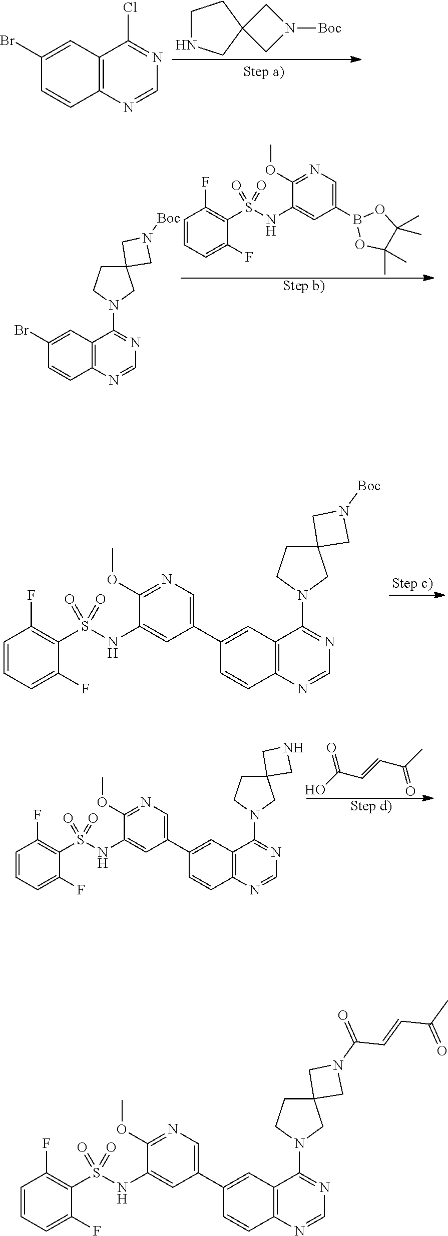

### Step a): Preparation of Tert-butyl 6-(6-bromoquinazolin-4-yl)-2,6-diazaspiro[3.4]octane-2-carboxylate

6-bromo-4-chloroquinazoline (500 mg, 2.053 mmol), tert-butyl 2,6-diazaspiro[3.4]octane-2-carboxylate (436 mg, 2.464 mmol) and triethylamine (623 mg, 6.159 mmol) were added to N,N-dimethylformamide (10 mL). After the addition was completed, the reaction mixture was heated to 60° C., stirred and reacted for 2 h. Upon completion of the reaction, the reaction was quenched by adding water (50 mL). The reaction mixture was extracted by adding dichloromethane (150 mL×2 times). The organic phases were combined, washed with saturated brine (100 mL), dried over anhydrous sodium sulfate and filtered. The filtrate was concentrated under reduced pressure. The crude product obtained was purified by silica gel column chromatography (Eluent: dichloromethane/methanol=100/1 to 50/1). Tert-butyl 6-(6-bromoquinazolin-4-yl)-2,6-diazaspiro[3.4]octane-2-carboxylate was obtained, yield: 83.9%; ESI-MS(m/z): 419.1 [M+H]⁺.

### Step b): Preparation of tert-butyl 6-(6-(5-((2,6-difluorophenyl)sulfonamido)-6-methoxypyridin-3-yl)quinazolin-4-yl)-2,6-diazaspiro[3.4]octane-2-carboxylate

Tert-butyl 6-(6-bromoquinazolin-4-yl)-2,6-diazaspiro[3.4]octane-2-carboxylate (400 mg, 0.954 mmol), 2,6-difluoro-N-(2-methoxy-5-(4,4,5,5-tetramethyl-1,3,2-dioxaboran-2-yl)pyridine-3-benzenesulfonamide (447 mg, 1.049 mmol), Pd(dppf)Cl₂ (140 mg, 0.191 mmol) and cesium carbonate (932 mg, 2.862 mmol) were added to dioxane/water mixed solvent (10 mL, v/v=10:1). After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (200 mL×2 times). The organic phases were combined, washed with saturated brine (100 mL), dried over anhydrous sodium sulfate and filtered. The filtrate was concentrated under reduced pressure. The crude product obtained was purified by silica gel column chromatography (Eluent: dichloromethane/methanol=100/1 to 50/1). tert-butyl 6-(6-(5-((2,6-difluorophenyl)sulfonamido)-6-methoxypyridin-3-yl)quinazolin-4-yl)-2,6-diazaspiro[3.4]octane-2-carboxylate was obtained, yield: 90.5%; ESI-MS(m/z): 639.2 [M+H]⁺.

### Step c): Preparation of N-(5-(4-(2,6-diazaspiro[3.4]octane-6-yl)quinazolin-6-yl)-2-methoxypyridin-3-yl)-2,6-difluorobenzenesulfonamide trifluoroacetate

tert-butyl 6-(6-(5-((2,6-difluorophenyl)sulfonamido)-6-methoxypyridin-3-yl)quinazolin-4-yl)-2,6-diazaspiro[3.4]octane-2-carboxylate (500 mg, 0.783 mmol) was dissolved in dichloromethane (4 mL). Under the ice bath condition, TFA (1.5 mL) was slowly added dropwise. After the addition was completed, the reaction mixture was stirred at room temperature for 1 h. Upon completion of the reaction, the reaction mixture was concentrated under reduced pressure. Methyl tert-butyl ether was added to the crude product. The mixture was stirred to precipitate solid and filtered. N-(5-(4-(2,6-diazaspiro[3.4]octane-6-yl)quinazolin-6-yl)-2-methoxypyridin-3-yl)-2,6-difluorobenzenesulfonamide trifluoroacetate was obtained, yield: 92.8%; ESI-MS(m/z): 539.2 [M+H]⁺.

### Step d): Preparation of (E)-2,6-difluoro-N-(2-methoxy-5-(4-(2-(4-oxopent-2-enoyl))-2,6-diazaspiro[3.4]octane-6-yl)quinazolin-6-yl)pyridin-3-yl)benzenesulfonamide

N-(5-(4-(2,6-diazaspiro[3.4]octane-6-yl)quinazolin-6-yl)-2-methoxypyridin-3-yl)-2,6-difluorobenzenesulfonamide trifluoroacetate (150 mg, 0.230 mmol) was dissolved in tetrahydrofuran (2 mL). The reaction system was cooled to −78° C., with N,N-diisopropylethylamine (178 mg, 1.380 mmol), (E)-4-oxopent-2-enoic acid (26 mg, 0.230 mmol) and 50% T₃P ethyl acetate solution (293 mg, 0.460 mmol) added successively. After the addition was completed, the reaction mixture was kept at −78° C. and stirred for 1 h. Upon completion of the reaction, the reaction solution was diluted by adding acetonitrile (0 mL) and purified by Prep-HPLC (Method 2). (E)-2,6-difluoro-N-(2-methoxy-5-(4-(2-(4-oxopent-2-enoyl))-2,6-diazaspiro[3.4]octane-6-yl)quinazolin-6-yl)pyridin-3-yl)benzenesulfonamide was obtained, yield: 19.1%; ¹H NMR (400 MHz, DMSO-d₆) δ 8.48 (s, 1H), 8.43 (s, 1H), 8.36 (s, 1H), 8.01-7.99 (m, 2H), 7.80 (d, J=8.0 Hz, 1H), 7.71-7.65 (m, 1H), 7.27-7.22 (m, 2H), 6.88 (d, J=16.0 Hz, 1H), 6.71 (d, J=16.0 Hz, 1H), 4.41 (d, J=8.0 Hz, 1H), 4.31 (d, J=8.0 Hz, 1H), 4.24-4.17 (m, 2H), 4.10-4.07 (m, 1H), 4.02-3.96 (m, 3H), 3.65 (s, 3H), 2.83 (s, 3H), 2.28-2.24 (m, 2H); ESI-MS(m/z): 635.0 [M+H]⁺.

Referring to the preparation method of Example 42 and using the corresponding raw materials, the compounds in the following examples were prepared.

| Number | Name | Structure | ¹H NMR and MS |
|---|---|---|---|
| Example 43 | 2,6-difluoro-N-(5- (4-(4-(2- fluoroacryloyl) piperazin-1- yl)quinazolin-6- yl)-2- methoxypyridin-3- yl) benzenesulfonamide | 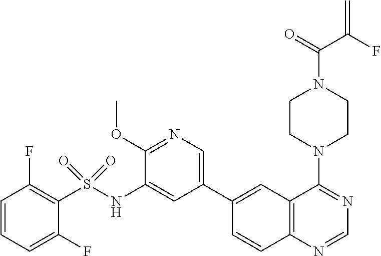 [SMILES: C=C(F)C(=O)N1CCN(c2ncnc3ccc(-c4cnc(OC)c(NS(=O)(=O)c5c(F)cccc5F)c4)cc23)CC1] | ¹H NMR (400 MHz, DMSO-d₆) δ 8.66 (s, 1H), 8.49 (s, 1H), 8.13-8.03 (m, 3H), 7.93-7.91 (m, 1H), 7.71-7.69 (m, 1H), 7.30-7.24 (m, 2H), 5.38-5.20 (m, 2H), 3.92-3.69 (m, 8H), 3.65 (s, 3H); ESI-MS(m/z): 585.0 [M + H]⁺. |
| Example 44 | (E)-2,6-difluoro- N-(2-methoxy-5- (4-(4-(4,4,4- trifluorobut-2- enoyl)piperazin-1- yl)quinazolin-6- yl)pyridin-3- yl) benzenesulfonamide | 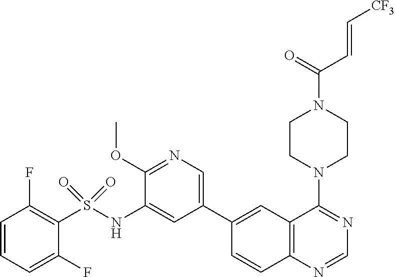 [SMILES: COc1ncc(-c2ccc3ncnc(N4CCN(C(=O)/C=C/C(F)(F)F)CC4)c3c2)cc1NS(=O)(=O)c1c(F)cccc1F] | ¹H NMR (400 MHz, DMSO-d₆) δ 10.59 (brs, 1H), 8.67 (s, 1H), 8.48 (s, 1H), 8.13-8.08 (m, 2H), 8.04 (d, J = 4.0 Hz, 1H), 7.93 (d, J = 8.0 Hz, 1H), 7.75-7.68 (m, 1H), 7.43-7.38 (m, 1H), 7.30-7.25 (t, 2H), 6.86-6.77 (m, 1H), 3.91-3.80 (m, 8H), 3.65 (s, 3H); ESI- MS (m/z): 635.0 [M + H]⁺. |
| Example 45 | (S)-2,6-difluoro- N-(5-(4-(4-(2- fluoroacryloyl)-2- methylpiperazin- 1-yl)quinazolin-6- yl)-2- methoxypyridin-3- yl) benzenesulfonamide | 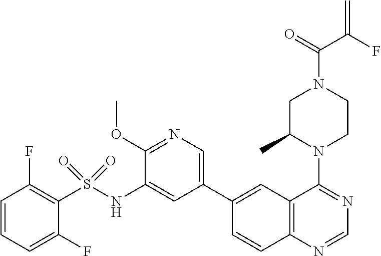 [SMILES: C=C(F)C(=O)N1CCN(c2ncnc3ccc(-c4cnc(OC)c(NS(=O)(=O)c5c(F)cccc5F)c4)cc23)[C@@H](C)C1] | ¹H NMR (400 MHz, DMSO-d₆) δ 10.61 (s, 1H), 8.69 (s, 1H), 8.47 (s, 1H), 8.11-8.04 (m, 3H), 7.94 (d, J = 8.0 Hz, 1H), 7.71 (s, 1H), 7.27 (s, 2H), 5.39-5.35 (m, 1H), 5.34-5.22 (m, 1H), 4.81 (s, 1H), 4.25-4.09 (m, 3H), 3.69 (s, 4H), 3.20 (d, J = 4.0 Hz, 2H), 1.35 (d, J = 4.0 Hz, 3H); ESI- MS(m/z): 599.2 [M + H]⁺. |
| Example 46 | (S,E)-2,6-difluoro- N-(2-methoxy-5- (4-(2-methyl-4-(4- oxopent-2- enoyl)piperazin-1- yl)quinazolin-6- yl)pyridin-3- yl) benzenesulfonamide | 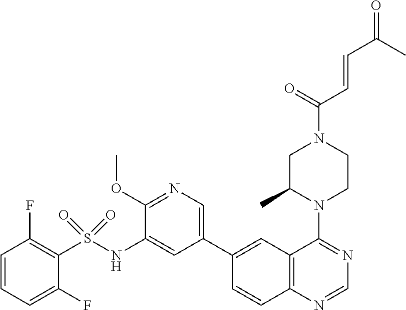 [SMILES: COc1ncc(-c2ccc3ncnc(N4CCN(C(=O)/C=C/C(C)=O)C[C@@H]4C)c3c2)cc1NS(=O)(=O)c1c(F)cccc1F] | ¹H NMR (400 MHz, DMSO-d₆) δ 8.63 (s, 1H), 8.35 (s, 1H), 8.06-8.02 (m, 2H), 7.96 (s, 1H), 7.90 (d, J = 8.0 Hz, 1H), 7.68-7.61 (m, 1H), 7.52-7.30 (m, 1H), 7.21 (t, J = 8.0 Hz, 2H), 6.76- 5.70 (m, 1H), 4.79-4.73 (m, 1H), 4.41-4.14 (m, 3H), 3.96 (d, J = 16.0 Hz, 1H), 3.65 (s, 4H), 3.22-3.11 (m, 1H), 2.36 (d, J = 4.0 Hz, 3H), 1.31- 1.28 (m, 3H); ESI-MS(m/z): 623.2 [M + H]⁺. |
| Example 47 | 2,6-difluoro-N-(5- (4-(2-(2- fluoroacryloyl)- 2,7- diazaspiro[3.5] nonan-7- yl)quinazolin-6- yl)-2- methoxypyridin-3- yl) benzenesulfonamide | 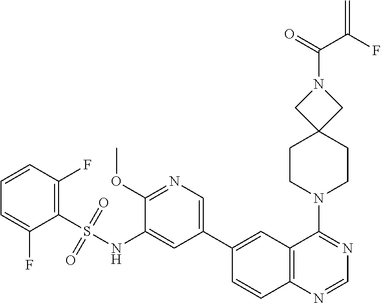 [SMILES: C=C(F)C(=O)N1CC2(CCN(c3ncnc4ccc(-c5cnc(OC)c(NS(=O)(=O)c6c(F)cccc6F)c5)cc34)CC2)C1] | ¹H NMR (400 MHz, DMSO-d₆) δ 10.59 (s, 1H), 8.62 (s, 1H), 8.40 (s, 1H), 8.09-8.01 (m, 2H), 7.98-7.97 (m, 1H), 7.88 (d, J = 8.4 Hz, 1H), 7.71- 7.67 (m, 1H), 7.25 (t, J = 9.2 Hz, 2H), 5.47 (dd, J = 48.4, 3.6 Hz, 1H), 5.29 (dd, J = 16.4, 3.6 Hz, 1H), 4.17 (d, J = 3.2 Hz, 2H), 3.79 (s, 2H), 3.75-3.73 (m. 4H), 3.66 (s, 3H), 1.95 (t, J = 5.2 Hz, 4H); ESI-MS(m/z): 625.0 [M + H]⁺. |
| Example 48 | 2,6-difluoro-N-(5- (4-(2-(2- fluoroacryloyl)- 2,6- diazaspiro[3.4] octan-6- yl)quinazolin-6- yl)-2- methoxypyridin-3- yl) benzenesulfonamide | 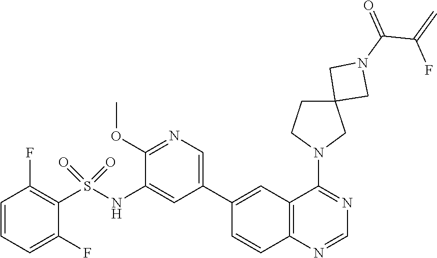 [SMILES: C=C(F)C(=O)N1CC2(CCN(c3ncnc4ccc(-c5cnc(OC)c(NS(=O)(=O)c6c(F)cccc6F)c5)cc34)C2)C1] | ¹H NMR (400 MHz, DMSO-d₆) δ 10.58 (s, 1H), 8.47 (s, 1H), 8.42 (s, 1H), 8.35 (s, 1H), 8.01-7.98 (m, 2H), 7.80 (d, J = 8.0 Hz, 1H), 7.72-7.65 (m, 1H), 7.27-7.22 (m, 2H), 5.52-5.39 (m, 1H), 5.30-5.25 (m, 1H), 4.48-4.45 (m, 1H), 4.36-4.32 (m, 1H), 4.23-4.16 (m, 2H), 4.10-4.07 (m, 1H), 4.01-3.96 (m, 3H), 3.66 (s, 3H), 2.29-2.20 (m, 2H); ESI-MS(m/z): 611.0 [M + H]⁺. |
| Example 49 | (E)-2,6-difluoro- N-(2-methoxy-5- (4-(2-(4-oxopent- 2-enoyl)-2,7- diazaspiro[3.5] nonan-7- yl)quinazolin-6- yl)pyridin-3- yl) benzenesulfonamide | 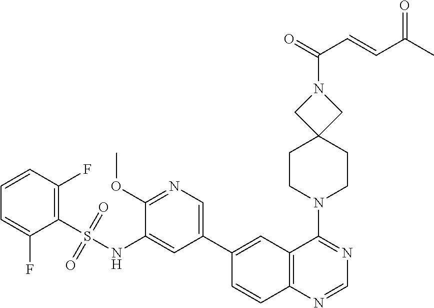 [SMILES: COc1ncc(-c2ccc3ncnc(N4CCC5(CC4)CN(C(=O)/C=C/C(C)=O)C5)c3c2)cc1NS(=O)(=O)c1c(F)cccc1F] | ¹H NMR (400 MHz, DMSO-d₆) δ 10.56 (s, 1H), 8.62 (s, 1H), 8.35 (s, 1H), 8.06-8.03 (m, 2H), 7.97-7.95 (m, 1H), 7.88 (d, J = 12.0 Hz, 1H), 7.67- 7.65 (m, 1H), 7.23 (t, J = 12.0 Hz, 2H), 6.93 (d, J = 16.0 Hz, 1H), 6.74 (d, J = 16.0 Hz, 1H), 4.14 (s, 2H), 3.79 (s, 2H), 3.76-7.72 (m, 4H), 3.67 (s, 3H), 2.34 (s, 3H), 1.96 (t, J = 5.2 Hz, 4H); ESI-MS(m/z): 649.0 [M + H]⁺. |
| Example 50 | N-(5-(4-(2- acryloyl-2,7- diazaspiro[3.5] nonan-7- yl)quinazolin-6- yl)-2- methoxypyridin-3- yl)-2,6- difluorobenzene- sulfonamide | 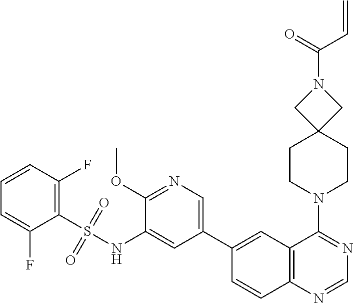 [SMILES: C=CC(=O)N1CC2(CCN(c3ncnc4ccc(-c5cnc(OC)c(NS(=O)(=O)c6c(F)cccc6F)c5)cc34)CC2)C1] | ¹H NMR (400 MHz, DMSO-d₆) δ 10.59 (s, 1H), 8.62 (s, 1H), 8.42 (s, 1H), 8.11-7.95 (m, 3H), 7.89 (d, J = 8.4 Hz, 1H), 7.74-7.7.67 (m, 1H), 7.27 (t, J = 9.6 Hz, 2H), 6.34 (dd, J = 17.2, 10.4 Hz, 1H), 6.11 (dd, J = 16.8, 2.4 Hz, 1H), 5.67 (dd, J = 10.4, 2.4 Hz, 1H), 4.02 (s, 2H), 3.75-3.73 (m, 6H), 3.66 (s, 3H), 1.94 (t, J = 5.2 Hz, 4H); ESI-MS(m/z): 607.0 [M + H]⁺. |
| Example 51 | N-(5-(4-((2S,5R)- 2,5-dimethyl-4- ((E)-4-oxopent-2- enoyl)piperazin-1- yl)quinazolin-6- yl)-2- methoxypyridin-3- yl)-2,6- difluorobenzene- sulfonamide | 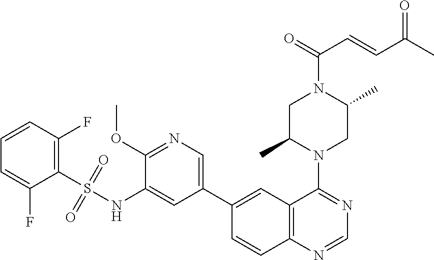 [SMILES: COc1ncc(-c2ccc3ncnc(N4C[C@@H](C)N(C(=O)/C=C/C(C)=O)C[C@@H]4C)c3c2)cc1NS(=O)(=O)c1c(F)cccc1F] | ¹H NMR (400 MHz, DMSO-d₆) δ 10.59 (s, 1H), 8.66 (s, 1H), 8.45 (s, 1H), 8.10-8.05 (m, 2H), 8.03-7.99 (m, 1H), 7.92 (d, J = 12.0 Hz, 1H), 7.74- 7.66 (m, 1H), 7.49-7.35 (m, 1H), 7.26 (t, J = 12.0 Hz, 2H), 6.78-6.72 (m, 1H), 4.88-4.78 (m, 1H), 4.77-4.44 (m, 1H), 4.21-4.08 (m, 1H), 3.91-3.81 (m, 2H), 3.65 (s, 3H), 3.54-3.42 (m, 1H), 2.37 (s, 3H), 1.31-1.15 (m, 6H); ESI- MS(m/z): 637.0 [M + H]⁺. |
| Example 52 | 2,6-difluoro-N-(5- (4-((2S,5R)-4-(2- fluoroacryloyl)- 2.5- dimethylpiperazin- 1-yl)quinazolin- 6-yl)-2- methoxypyridin-3- yl) benzenesulfonamide | 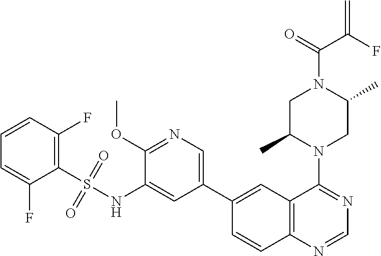 [SMILES: C=C(F)C(=O)N1C[C@H](C)N(c2ncnc3ccc(-c4cnc(OC)c(NS(=O)(=O)c5c(F)cccc5F)c4)cc23)C[C@H]1C] | ¹H NMR (400 MHz, DMSO-d₆) δ 10.60 (s, 1H), 8.65 (s, 1H), 8.46 (s, 1H), 8.09-8.06 (m, 2H), 8.03-8.01 (m, 1H), 7.92 (d, J = 12.0 Hz, 1H), 7.74- 7.66 (m, 1H), 7.26 (t, J = 8.0 Hz, 2H), 5.36-5.16 (m, 2H), 4.95-4.60 (m, 2H), 4.33-4.06 (m, 2H), 3.90-3.71 (m, 2H), 3.65 (s, 3H), 1.30-1.21 (m, 6H); ESI- MS(m/z): 613.0 [M + H]⁺. |
| Example 53 | N-(5-(4-((2S,5R)- 4-acryloyl-2,5- dimethylpiperazin- 1-yl)quinazolin- 6-yl)-2- methoxypyridin-3- yl)-2,6- difluorobenzene- sulfonamide | 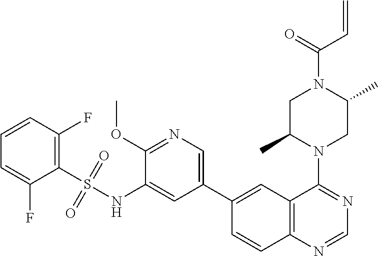 [SMILES: C=CC(=O)N1C[C@H](C)N(c2ncnc3ccc(-c4cnc(OC)c(NS(=O)(=O)c5c(F)cccc5F)c4)cc23)C[C@H]1C] | ¹H NMR (400 MHz, DMSO-d₆) δ 10.60 (s, 1H), 8.65 (s, 1H), 8.49-8.47 (m, 1H), 8.10-8.06 (m, 2H), 8.05-8.01 (m, 1H), 7.93-7.89 (m, 1H), 7.76-7.68 (m, 1H), 7.28 (t, J = 8.0 Hz, 2H), 6.88-6.76 (m, 1H), 6.20-6.14 (m, 1H), 5.76-5.71 (m, 1H), 4.85-4.40 (m, 2H), 4.20-3.75 (m, 4H), 3.65 (s, 3H), 1.29- 1.20 (m, 6H); ESI-MS(m/z): 595.0 [M + H]⁺. |
| Example 54 | 2,6-difluoro-N-(2- methoxy-5-(4-(4- (3-oxocyclohex-1- ene-1- carbonyl)piperazin- 1-yl)quinazolin- 6-yl)pyridin-3- yl) benzenesulfonamide | 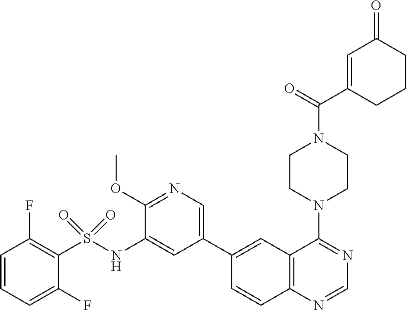 [SMILES: COc1ncc(-c2ccc3ncnc(N4CCN(C(=O)C5=CC(=O)CCC5)CC4)c3c2)cc1NS(=O)(=O)c1c(F)cccc1F] | ¹HNMR (400 MHz, DMSO-d₆) δ 10.59 (s, 1H), 8.68 (s, 1H), 8.49 (s, 1H), 8.11-8.09 (m, 2H), 8.05 (s, 1H), 7.92 (d, J = 8 Hz, 1H), 7.75-7.72 (m, 1H), 7.27 (t, J = 12 Hz, 2H), 5.94 (s, 1H), 3.95-3.90 (m, 4H), 3.75-3.69 (m, 4H), 3.64 (s, 3H), 3.35-3.34 (m, 2H), 2.41-22.40 (m, 2H), 2.07-2.01 (m, 2H); ESI-MS(m/z): 635.0 [M + H]⁺. |
| Example 55 | N-(5-(4-(4-(1- acryloylazetidin- 3-yl)piperazin-1- yl)quinazolin-6- yl)-2- methoxypyridin-3- yl)-2,4- difluorobenzene- sulfonamide | 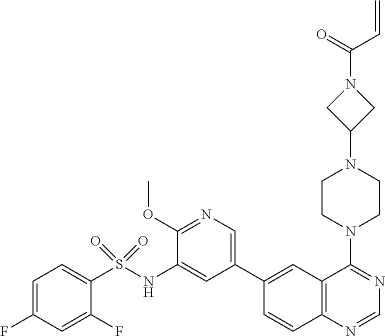 [SMILES: C=CC(=O)N1CC(N2CCN(c3ncnc4ccc(-c5cnc(OC)c(NS(=O)(=O)c6ccc(F)cc6F)c5)cc34)CC2)C1] | ¹H NMR (400 MHz, DMSO-d₆) δ 10.41 (s, 1H), 8.64 (s, 1H), 8.45 (s, 1H), 8.06-8.05 (m, 2H), 7.95 (s, 1H), 7.83-7.80 (m, 1H), 7.78-7.76 (m, 1H), 7.53-7.51 (m, 1H), 7.25-7.20 (m, 1H), 6.35-6.31 (m, 1H), 6.08-6.07 (m, 1H), 5.67 (d, J = 12.0 Hz, 1H), 4.26-4.23 (m, 1H), 4.13-4.10 (m, 1H), 4.01-3.97 (m, 1H), 3.83-3.79 (m, 5H), 3.69 (s, 3H), 3.28-3.25 (m, 1H), 2.58-2.52 (m, 4H); ESI-MS(m/z): 622.0 [M + H]⁺. |
| Example 56 | (E)-2,4-difluoro- N-(2-methoxy-5- (4-(4-(1-(4- oxopent-2- enoyl)azetidin-3- yl)piperazin-1- yl)quinazolin-6- yl)pyridin-3- yl)benzenesulfon- amide | 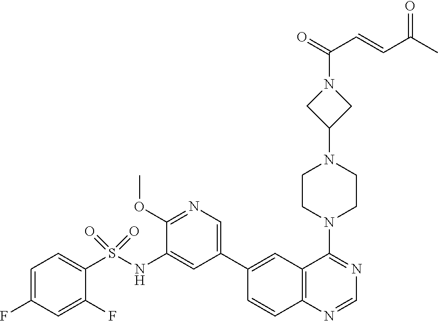 [SMILES: COc1ncc(-c2ccc3ncnc(N4CCN(C5CN(C(=O)/C=C/C(C)=O)C5)CC4)c3c2)cc1NS(=O)(=O)c1ccc(F)cc1F] | ¹HNMR (400 MHz, DMSO-d₆) δ 10.41 (s, 1H), 8.64 (s, 1H), 8.45 (s, 1H), 8.06-8.05 (m, 2H), 7.91-7.80 (m, 2H), 7.78-7.76 (m, 1H), 7.53-7.51 (m, 1H), 7.25-7.20 (m, 1H), 6.91 (d, J = 12 Hz, 1H), 6.71 (d, J = 12 Hz, 1H), 4.38-4.36 (m, 1H), 4.28-4.24 (m, 1H), 4.02-3.97 (m, 1H), 3.89-3.70 (m, 5H), 3.69 (s, 3H), 2.83-2.80 (m, 1H), 2.58- 2.52 (m, 4H), 2.23 (s, 3H); ESI- MS(m/z): 664.0 [M + H]⁺. |
| Example 57 | 2,4-difluoro-N-(5- (4-(4-(1-(2- fluoroacryloyl) azetidin-3- yl)piperazin-1- yl)quinazolin-6- yl)-2- methoxypyridin-3- yl)benzenesulfon- amide | 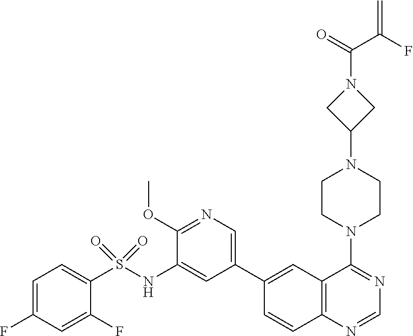 [SMILES: C=C(F)C(=O)N1CC(N2CCN(c3ncnc4ccc(-c5cnc(OC)c(NS(=O)(=O)c6ccc(F)cc6F)c5)cc34)CC2)C1] | ¹HNMR (400 MHz, DMSO-d₆) δ 10.41 (s, 1H), 8.64 (s, 1H), 8.45 (s, 1H), 8.06-8.05 (m, 2H), 7.91-7.80 (m, 2H), 7.78-7.76 (m, 1H), 7.53-7.51 (m, 1H), 7.25-7.20 (m, 1H), 5.53-5.40 (m, 1H), 5.31-5.20 (m, 1H), 4.90-4.81 (m, 1H), 4.76-4.73 (m, 1H), 4.07-4.03 (m, 1H), 3.87-3.81 (m, 5H), 3.69 (s, 3H), 3.28-3.25 (m. 1H), 2.58-2.52 (m, 4H); ESI-MS(m/z): 640.0 [M + H]⁺. |
| Example 58 | 2,4-difluoro-N-(2- methoxy-5-(4-(8- ((E)-4-oxopent-2- enoyl)-3,8- diazabicyclo[3.2.1] octan-3- yl)quinazolin-6- yl)pyridin-3- yl)benzenesulfon- amide | 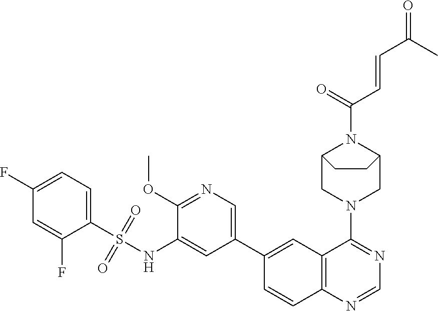 [SMILES: COc1ncc(-c2ccc3ncnc(N4CC5CCC(C4)N5C(=O)/C=C/C(C)=O)c3c2)cc1NS(=O)(=O)c1ccc(F)cc1F] | ¹H NMR (400 MHz, DMSO-d₆) δ 10.31 (brs, 1H), 8.60 (s, 1H), 8.45 (s, 1H), 8.09-8.05 (m, 2H), 8.00-7.98 (m, 1H), 7.88 (d, J = 8.0 Hz, 1H), 7.79- 7.73 (m, 1H), 7.60-7.55 (m, 1H), 7.41 (d, J = 16.0 Hz, 1H), 7.24-7.19 (m, 1H), 6.84 (d, J = 16.0 Hz, 1H), 4.80- 4.74 (m, 2H), 4.55-4.46 (m, 2H), 3.67 (s, 3H), 3.63-3.57 (m, 2H), 2.93 (s, 3H), 1.96-1.79 (m, 4H); ESI- MS(m/z): 635.0 [M + H]⁺. |

### Example 59

### Preparation of 2,4-difluoro-N-(5-(4-(4-(2-fluoroacryloyl)piperazin-1-yl)quinazolin-6-yl)-2-methoxypyridin-3-yl)benzenesulfonamide

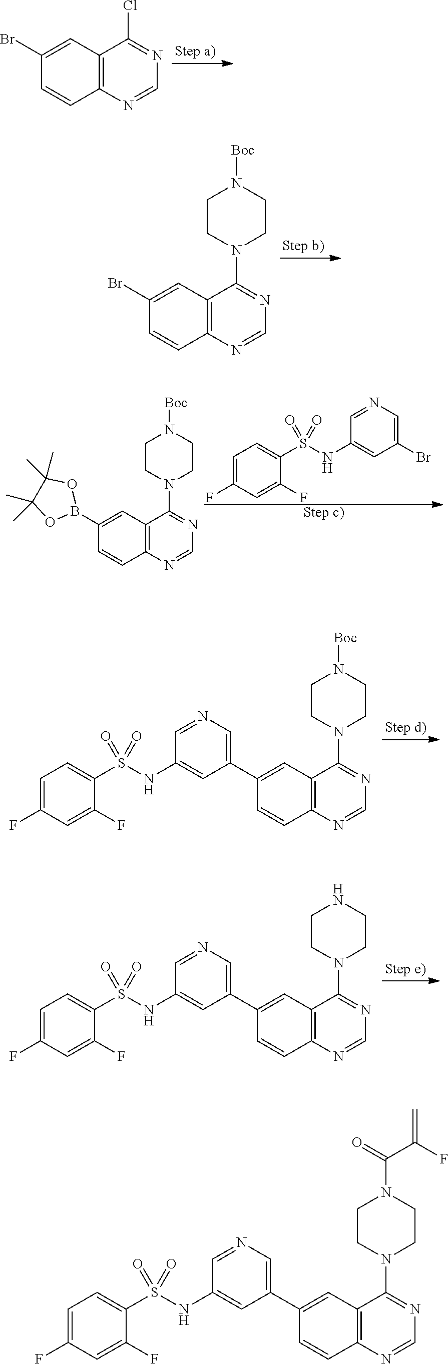

### Step a): Preparation of tert-butyl 4-(6-bromoquinazolin-4-yl)piperazine-1-carboxylate

6-Bromo-4-chloroquinazoline (1.9 g, 7.820 mmol) and tert-butyl piperazine-1-carboxylate (1.8 g, 9.380 mmol) were dissolved in dimethyl sulfoxide (30 mL), with triethylamine (2.4 g, 23.470 mmol) added. After the addition was completed, the reaction mixture was heated to 60° C. and stirred for 6 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated under reduced pressure. The crude product obtained was purified by silica gel column chromatography (Eluent: dichloromethane/methanol=50/1 to 10/1). Tert-butyl 4-(6-bromoquinazolin-4-yl)piperazine-1-carboxylate was obtained, yield: 65.4%. ESI-MS(m/z): 393.1 [M+H]⁺.

### Step b): Preparation of tert-butyl 44 (6-(4,4,5,5-tetramethyl-1,3,2-dioxaborolan-2-yl)quinazolin-4-yl)piperadine-1-carboxylate

Tert-butyl 4-(6-bromoquinazolin-4-yl)piperazine-1-carboxylate (2.0 g, 5.10 mmol), bis(pinacolato)diboron (1.9 g, 7.640 mmol), Pd(dppf)Cl₂ (373 mg, 0.510 mmol) and potassium acetate (1.5 g, 15.310 mmol) were dissolved in dioxane (30 mL). After the addition was completed, in the presense of protective nitrogen, the reaction mixture was kept at 110° C. and stirred for 4 h. Upon completion of the reaction, the reaction mixture was filtered. The filtrate was concentrated through pressure concentration. The crude product obtained was purified by silica gel column chromatography (Eluent: dichloromethane/methanol=50/1 to 10/1). Tert-butyl 4-(6-(4,4,5,5-tetramethyl-1,3,2-dioxaborolan-2-yl)quinazolin-4-yl)piperazine-1-carboxylate was obtained, yield: 62.5%; ESI-MS(m/z): 441.3 [M+H]⁺.

### Step c): Preparation of tert-butyl 4-(6-(5-((2,4-difluorophenyl)sulfonamido)-6-methoxypyridin-3-yl)quinazolin-4-yl)piperazine-1-carboxylate

N-(5-bromo-2-methoxypyridin-3-yl)-2,4-difluorobenzenesulfonamide (300 mg, 0.790 mmol), tert-butyl 4-(6-(4,4,5,5-tetramethyl-1,3,2-dioxaborolan-2-yl)quinazolin-4-yl)piperazine-1-carboxylate (698 mg, 1.580 mmol), Pd(dppf)Cl₂ (58 mg, 0.080 mmol) and cesium carbonate (779 mg, 2.390 mmol) were dissolved in dioxane/water (4:1, 10 mL). After the addition was completed, in the presense of protective nitrogen, the reaction mixture was kept at 110° C. and stirred for 4 h. Upon completion of the reaction, the reaction was quenched by adding water (50 mL). The reaction mixture was extracted with ethyl acetate (100 mL 2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=1/1 to 1/3). Tert-butyl 4-(6-(5-((2,4-difluorophenyl)sulfonamido)-6-methoxypyridin-3-yl)quinazolin-4-yl)piperazine-1-carboxylate was obtained, yield: 82.5%. ESI-MS(m/z): 613.1 [M+H]⁺.

### Step d): Preparation of 2,4-difluoro-N-(2-methoxy-5-(4-(piperazin-1-yl)quinazolin-6-yl)pyridin-3-yl)benzenesulfonamide

Tert-butyl 4-(6-(5-((2,4-difluorophenyl)sulfonamido)-6-methoxypyridin-3-yl)quinazolin-4-yl)piperazine-1-carboxylate (400 mg, 0.650 mmol) was dissolved in dichloromethane (8 mL). Under the ice bath condition, trifluoroacetic acid (2 mL) was slowly added dropwise. After the addition was completed, the reaction mixture was stirred at room temperature for 1 h. Upon completion of the reaction, the reaction mixture was concentrated under reduced pressure. 2,4-difluoro-N-(2-methoxy-5-(4-(piperazin-1-yl)quinazolin-6-yl)pyridin-3-yl)benzenesulfonamide was obtained. ESI-MS(m/z): 513.1 [M+H]⁺.

### Step e): Preparation of 2,4-difluoro-N-(5-(4-(4-(2-fluoroacryloyl)piperazin-1-yl)quinazolin-6-yl)-2-methoxypyridin-3-yl)benzenesulfonamide

2-Fluoroacrylic acid (35 mg, 0.388 mmol) and 2,4-difluoro-N-(2-methoxy-5-(4-(piperazin-1-yl)quinazolin-6-yl)pyridin-3-yl)benzenesulfonamide trifluoroacetate (80 mg, 0.128 mmol) were dissolved in THF (2 mL). The reaction system was cooled to −78° C., with N,N-diisopropylethylamine (99 mg, 0.766 mmol) and T₃P (244 mg, 0.383 mmol, 50/o wt) added successively. After the addition was completed, the reaction mixture was kept at −78° C. and stirred for 1 h. Upon completion of the reaction, the reaction was quenched by adding water (10 mL), and the reaction mixture was extracted with dichloromethane (30 mL×2 times). The organic phases were combined, dried over anhydrous sodium sulfate and filtered. The filtrate was concentrated under reduced pressure. The crude product obtained was purified by Prep-HPLC (Method 2), 2,4-difluoro-N-(5-(4-(4-(2-fluoroacryloyl)piperazin-1-yl)quinazolin-6-yl)-2-methoxypyridin-3-yl)benzenesulfonamide was obtained, yield: 12.9%; ¹H NMR (400 MHz, DMSO-d₆) δ 10.32 (s, 1H), 8.66 (s, 1H), 8.46 (s, 1H), 8.13-8.07 (m, 2H), 8.02-7.99 (m, 1H), 7.91 (d, J=12.0 Hz, 1H), 7.81-7.74 (m, 1H), 7.58-7.51 (m, 1H), 7.24-7.18 (m, 1H), 5.38-5.19 (m, 2H), 3.92-3.88 (m, 4H), 3.82-3.76 (m, 4H), 3.68 (s, 3H); ESI-MS(m/z): 585.0 [M+H]⁺.

Referring to the preparation method of Example 59 and using the corresponding raw materials, the compounds in the following examples were prepared.

| Num- | | | |
|---|---|---|---|
| ber | Name | Structure | ¹H NMR and MS |
| Ex- ample 60 | 2,4-difluoro-N- (5-(4-(2-(2- fluoroacryloyl)- 2,6- diazaspiro[3.4] octan-6-yl) quinazolin- 6-yl)-2- methoxy- pyridin-3-yl) benzene- sulfonamide | 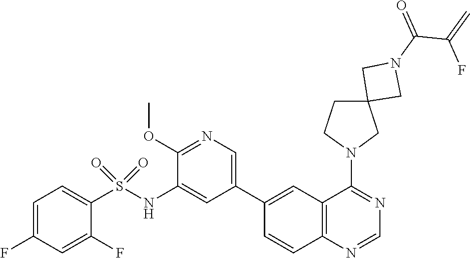 [SMILES: C=C(F)C(=O)N1CC2(CCN(c3ncnc4ccc(-c5cnc(OC)c(NS(=O)(=O)c6ccc(F)cc6F)c5)cc34)C2)C1] | ¹H NMR (400 MHz, DMSO-d₆) δ 10.30 (brs, 1H), 8.48 (s, 1H), 8.46 (s, 1H), 8.36 (s, 1H), 8.02 (s, 1H), 8.00 (s, 1H), 7.81-7.74 (m, 2H), 7.56 (t, J = 12.0 Hz, 1H), 7.20 (t, J = 12.0 Hz, 1H), 5.52 (d, J = 48.0 Hz, 1H), 5.30 (d, J = 16 Hz, 1H), 4.48-4.44 (m, 1H), 4.36-4.32 (m, 1H), 4.24-4.17 (m, 2H), 4.10 (d, J = 12.0 Hz, 1H), 4.02-3.96 (m, 3H), 3.68 (s, 3H), 2.28-2.23 (m, 2H); ESI-MS(m/z): 611.0 [M + H]⁺. |
| Ex- ample 61 | (E)-N-(5-(4- (2-(3-(1H- 1,2,3-triazol- 1-yl)acryloyl)- 2,6-diazaspiro [3.4]octan-6-yl) quinazolin- 6-yl)-2- methoxy- pyridin-3- yl)-2,4- difluoro- benzene- sulfonamide | 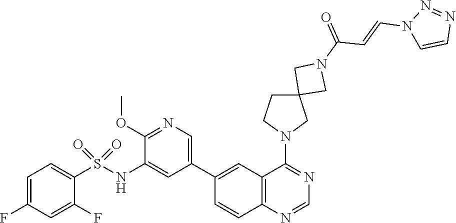 [SMILES: COc1ncc(-c2ccc3ncnc(N4CCC5(CN(C(=O)/C=C/n6ccnn6)C5)C4)c3c2)cc1NS(=O)(=O)c1ccc(F)cc1F] | ¹H NMR (400 MHz, DMSO-d₆) δ 10.31 (s, 1H), 8.67 (s, 1H), 8.48 (s, 1H), 8.38-8.35 (m, 2H), 8.13 (d, J = 16.0 Hz, 1H), 8.00-7.92 (m, 3H), 7.81-7.74 (m, 2H), 7.53-7.48 (m, 1H), 7.21-7.16 (m, 1H), 6.90 (d, J = 12.0 Hz, 1H), 4.41-4.36 (m, 1H), 4.30 (d, J = 8.0 Hz, 1H), 4.25-4.19 (m, 2H), 4.10 (d, J = 8.0 Hz, 1H), 4.02-3.98 (m, 3H), 3.69 (s, 3H), 2.29-2.26 (m, 2H); ESI-MS(m/z): 660.0 [M + H]⁺. |
| Ex- ample 62 | (E)-2,4- difluoro-N-(2- methoxy-5-(4- (4-(3-(oxazol-2- yl)acryloyl) piperazin- 1-yl)quinazolin- 6-yl) pyridin-3-yl) benzene- sulfonamide | 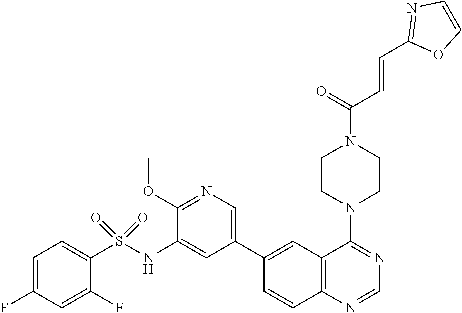 [SMILES: COc1ncc(-c2ccc3ncnc(N4CCN(C(=O)/C=C/c5ncco5)CC4)c3c2)cc1NS(=O)(=O)c1ccc(F)cc1F] | ¹H NMR (400 MHz, DMSO-d₆) δ 10.33 (s, 1H), 8.66 (s, 1H), 8.48 (d, J = 2.4 Hz, 1H), 8.24 (s, 1H), 8.14 (d, J = 2.0 Hz, 1H), 8.09 (dd, J = 8.4, 2.0 Hz, 1H), 8.02 (d, J = 2.4 Hz, 1H), 7.91 (d, J = 8.8 Hz, 1H), 7.77 (td, J = 8.4, 6.4 Hz, 1H), 7.59-7.75 (m, 1H), 7.46-7.43 (m, 2H), 7.30-7.17 (m, 2H), 3.93-3.84 (m, 8H), 3.68 (s, 3H); ESI- MS(m/z): 634.0 [M + H]⁺. |
| Ex- ample 63 | 2,4-difluoro- N-(2-methoxy- 5-(4-(4- (3-methyl-2- methylene- butanoyl) piperazin-1- yl)quinazolin-6- yl)pyridin-3-yl) benzene- sulfonamide | 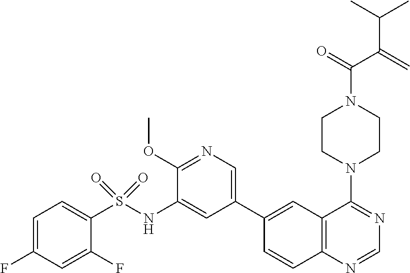 [SMILES: C=C(C(=O)N1CCN(c2ncnc3ccc(-c4cnc(OC)c(NS(=O)(=O)c5ccc(F)cc5F)c4)cc23)CC1)C(C)C] | ¹H NMR (400 MHz, DMSO-d₆) δ 10.33 (brs, 1H), 8.66 (s, 1H), 8.46 (s, 1H), 8.10 (s, 1H), 8.08 (d, J = 2.0 Hz, 1H), 8.02 (d, J = 2.4 Hz, 1H), 7.92 (d, J = 8.4 Hz, 1H), 7.80-7.74 (m, 1H), 7.59-7.53 (t, 1H), 7.24-7.19 (t, 1H), 5.18 (s, 1H), 5.04 (s, 1H), 3.82-3.75 (m, 8H), 3.68 (s, 3H), 2.62-2.55 (m, 1H), 1.05 (d, J = 6.8 Hz, 6H); ESI-MS (m/z): 609.0 [M + H]⁺. |
| Ex- ample 64 | (E)-2,4- difluoro-N-(2- methoxy-5-(4- (4-(4- methoxybut- 2-enoyl) piperazin-1-yl) quinazolin-6- yl)pyridin-3-yl) benzene- sulfonamide | 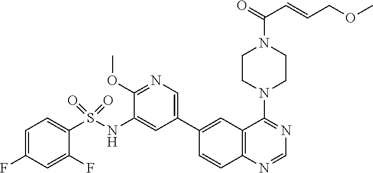 [SMILES: COC/C=C/C(=O)N1CCN(c2ncnc3ccc(-c4cnc(OC)c(NS(=O)(=O)c5ccc(F)cc5F)c4)cc23)CC1] | ¹H NMR (400 MHz, DMSO-d₆) δ 10.34 (brs, 1H), 8.65 (s, 1H), 8.47 (s, 1H), 8.13-8.08 (t, 2H), 8.022 (d, J = 4.0 Hz, 1H), 7.92 (d, J = 8.0 Hz, 1H), 7.80-7.74 (m, 1H), 7.59-7.54 (t, 1H), 7.24-7.19 (t, 1H), 6.76-6.70 (m, 1H), 6.66-6.61 (m, 1H), 4.09 (d, J = 4.0 Hz, 2H), 3.87-3.78 (m, 8H), 3.68 (s, 3H), 3.31(s, 3H); ESI-MS (m/z): 611.0 [M + H]⁺. |
| Ex- ample 65 | 2,4-difluoro- N-(2-methoxy- 5-(4-(4-(2- (methoxy- methyl) acryloyl) piperazin-1-yl) quinazolin-6- yl)pyridin-3-yl) benzene- sulfonamide | 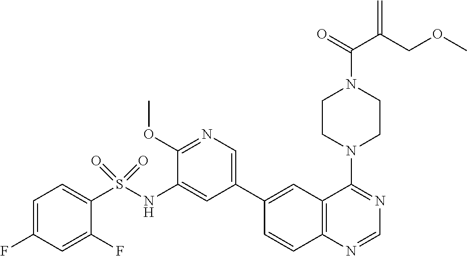 [SMILES: C=C(COC)C(=O)N1CCN(c2ncnc3ccc(-c4cnc(OC)c(NS(=O)(=O)c5ccc(F)cc5F)c4)cc23)CC1] | ¹H NMR (400 MHz, DMSO-d₆) δ 8.64 (s, 1H), 7.93-7.89 (m, 1H), 7.89- 7.78 (m, 3H), 7.78-7.75 (m, 1H), 7.57 (d, J = 2.0 Hz, 1H), 7.23-7.11 (m, 1H), 7.09-6.98 (m, 1H), 6.06 (s, 1H), 5.53-5.40 (m, 1H), 5.36-5.23 (m, 1H), 4.05 (s, 2H), 3.80 (s, 3H), 3.75 (s, 8H), 3.28 (s, 3H); ESI-MS (m/z): 611.2 [M + H]⁺. |
| Ex- ample 66 | 2,4-difluoro- N-(2-methoxy- 5-(4-(4- (2-methoxy- acryloyl) piperazin-1- yl)quinazolin-6- yl)pyridin-3-yl) benzene- sulfonamide | 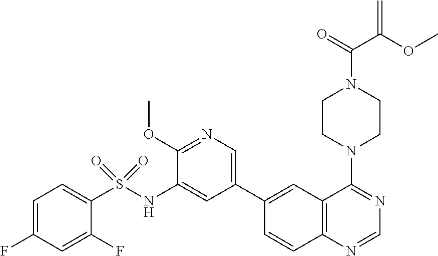 [SMILES: C=C(OC)C(=O)N1CCN(c2ncnc3ccc(-c4cnc(OC)c(NS(=O)(=O)c5ccc(F)cc5F)c4)cc23)CC1] | ¹H NMR (400 MHz, DMSO-d₆) δ 10.34 (s, 1H), 8.66 (s, 1H), 8.47 (s, 1H), 8.12-8.07 (m, 2H), 8.02 (d, J = 8.0 Hz, 1H), 7.93-7.89 (m, 1H), 7.80- 7.73 (m, 1H), 7.60-7.53 (m, 1H), 7.24-7.18 (m, 1H), 4.47-4.40 (m, 2H), 3.86-3.82 (m, 4H), 3.72-3.67 (m, 7H), 3.60 (s, 3H); ESI-MS (m/z): 597.0 [M + H]⁺. |
| Ex- ample 67 | methyl (E)-4-(4- (6-(5-((2,4- difluorophenyl) sulfonamido)- 6-methoxy- pyridin-3- yl)quinazolin- 4-yl)piperazin- 1-yl)-4-oxobut- 2-enoate | 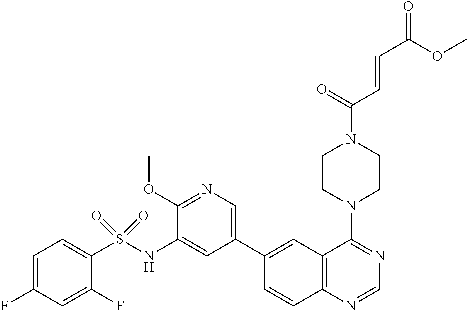 [SMILES: COC(=O)/C=C/C(=O)N1CCN(c2ncnc3ccc(-c4cnc(OC)c(NS(=O)(=O)c5ccc(F)cc5F)c4)cc23)CC1] | ¹H NMR (400 MHz, DMSO-d₆) δ 10.33 (s, 1H), 8.67 (s, 1H), 8.45 (s, 1H), 8.14-8.05 (m, 2H), 8.01 (d, J = 2.0 Hz, 1H), 7.92 (d, J = 8.4 Hz, 1H), 7.83-7.71 (m, 1H), 7.62-7.50 (m, 1H), 7.26-7.15 (m, 1H), 6.85 (d, J = 12.0 Hz, 1H), 6.12 (d, J = 12.0 Hz, 1H), 3.89-3.79 (m, 4H), 3.79-3.71 (m, 2H), 3.70-3.64 (m, 6H), 3.63-3.55 (m, 2H); ESI-MS (m/z): 625.2 [M + H]⁺. |
| Ex- ample 68 | 2,4-difluoro- N-(5-(4- ((2S,5R)-4-(2- fluoroacryloyl)- 2,5- dimethyl- piperazin-1-yl) quinazolin-6- yl)-2-methoxy- pyridin-3-yl) benzene- sulfonamide | 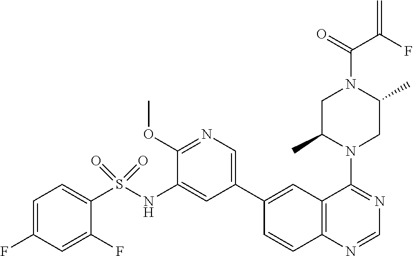 [SMILES: C=C(F)C(=O)N1C[C@H](C)N(c2ncnc3ccc(-c4cnc(OC)c(NS(=O)(=O)c5ccc(F)cc5F)c4)cc23)C[C@H]1C] | ¹H NMR (400 MHz, DMSO-d₆) δ 10.33 (brs, 1H), 8.65 (s, 1H), 8.49 (d, J = 4.0 Hz, 1H), 8.10-8.07 (m, 2H), 8.02 (d, J = 4.0 Hz, 1H), 7.93-7.90 (m, 1H), 7.80-7.74 (m, 1H), 7.62 (t, J = 8.0 Hz, 1H), 7.20 (t, J = 8.0 Hz, 1H), 5.34-5.18 (m, 2H), 4.85 (m, 1H), 4.63-4.30 (m, 1H), 3.81-3.77 (m, 1H), 3.72-3.49 (m, 1H), 3.68 (s, 3H), 1.29- 1.23 (m, 6H); ESI-MS(m/z): 613.0 [M + H]⁺. |
| Ex- ample 69 | 2,4-difluoro- N-(5- (4-(4-(3- hydroxy- benzoyl) piperazin-1- yl)quinazolin- 6-yl)-2- methoxy- pyridin-3-yl) benzene- sulfonamide | 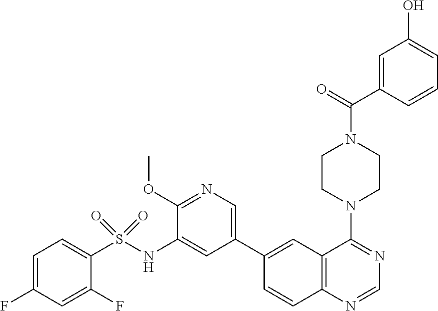 [SMILES: COc1ncc(-c2ccc3ncnc(N4CCN(C(=O)c5cccc(O)c5)CC4)c3c2)cc1NS(=O)(=O)c1ccc(F)cc1F] | ¹H NMR (400 MHz, DMSO-d₆) δ 10.33 (s, 1H), 9.72 (s, 1H), 8.66 (s, 1H), 8.46 (d, J = 4.0 Hz, 1H), 8.10- 8.07 (m, 2H), 8.00 (s, 1H), 7.91 (d, J = 8.0 Hz, 1H), 7.80-7.74 (m, 1H), 7.58-7.52 (m, 1H), 7.28-7.18 (m, 2H), 6.88-6.82 (m, 3H), 3.96-3.75 (m, 6H), 3.68-3.62(m, 5H), ESI-MS(m/z): 633.0 [M + H]⁺. |

### Example 70

### Preparation of 2,4-difluoro-N-(2-methoxy-5-(4-(1-oxo-8-azaspiro[4,5]dec-2-en-8-yl)quinazolin-6-yl)pyridin-3-yl)benzenesulfonamide

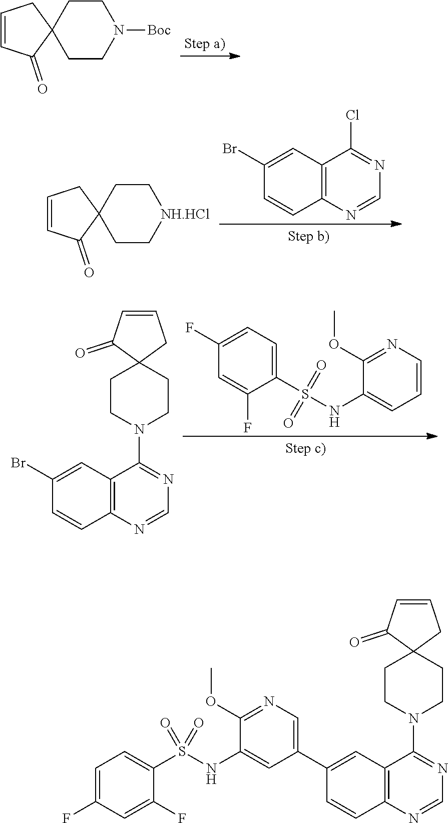

### Step a): Preparation of 8-azaspiro[4,5]dec-2-en-1-one hydrochloride

Tert-butyl 1-oxo-8-azaspiro[4,5]dec-2-ene-8-carboxylate (260 mg, 1.032 mmol) and tetrahydrofuran (2 mL) were added to the reaction flask, stirred to dissolve. At 0° C. under the condition of stirring, HCl/1,4-dioxane solution (4 M, 4 mL) was slowly added to the system. After the addition was completed, the reaction mixture was naturally heated to room temperature and reacted for 1 h. After completion of the reaction, the reaction mixture was concentrated under reduced pressure. The residue was used directly in the next reaction without purification. ESI-MS (m/z): 152.2 [M+H]⁺.

### Step b-c): Preparation of 2,4-difluoro-N-(2-methoxy-5-(4-(1-oxo-8-azaspiro[4,5]dec-2-en-8-yl)quinazolin-6-yl pyridin-3-yl)benzenesulfonamide

Referring to the preparation method of Example 42, Step a-b, 2,4-difluoro-N-(2-methoxy-5-(4-(1-oxo-8-azaspiro[4,5]dec-2-en-8-yl)quinazolin-6-yl)pyridin-3-yl)benzenesulfonamide was obtained, yield: 69.8%; ¹H NMR (400 MHz, DMSO-d₆) δ 10.32 (s, 1H), 8.64 (s, 1H), 8.38 (s, 1H), 8.08-8.04 (m, 2H), 7.95-7.88 (m, 3H), 7.80-7.75 (m, 1H), 7.56-7.51 (m, 1H), 7.23-7.18 (m, 1H), 6.23-6.20 (m, 1H), 4.36 (d, J=12.0 Hz, 2H), 3.69 (s, 3H), 3.42 (d, J=12.0 Hz, 2H), 2.78 (s, 2H), 1.89-1.81 (m, 2H), 1.43 (d, J=12.0 Hz, 2H); ESI-MS (m/z): 578.6 [M+H]⁺.

### Example 71

### Preparation of 2,6-difluoro-N-(2-methoxy-5-(4-(1-oxo-8-azaspiro[4,5]dec-2-en-8-yl)quinazolin-6-yl)pyridin-3-yl)benzenesulfonamide

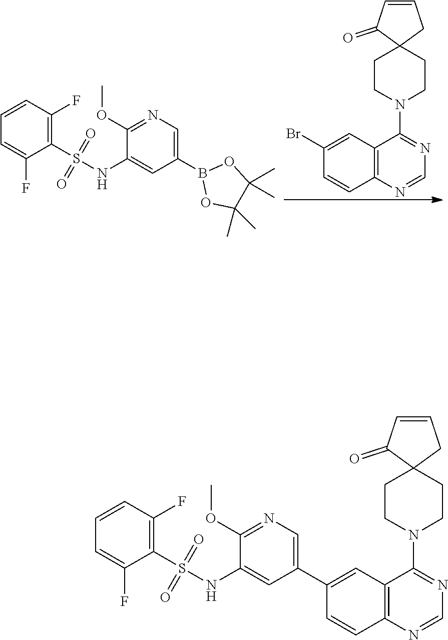 [SMILES: COc1ncc(B2OC(C)(C)C(C)(C)O2)cc1NS(=O)(=O)c1c(F)cccc1F ; O=C1C=CCC12CCN(c1ncnc3ccc(Br)cc13)CC2 ; COc1ncc(-c2ccc3ncnc(N4CCC5(CC=CC5=O)CC4)c3c2)cc1NS(=O)(=O)c1c(F)cccc1F]

Referring to the preparation method of Example 42, Step b, 2,6-difluoro-N-(2-methoxy-5-(4-(1-oxo-8-azaspiro[4,5]dec-2-en-8-yl)quinazolin-6-yl)pyridin-3-yl)benzenesulfonamide was obtained, yield: 42.2%. ¹H NMR (400 MHz, DMSO-dₕ) δ 10.57 (s, 1H), 8.68 (s, 1H), 8.39 (s, 1H), 8.09-8.07 (m, 2H), 7.99-7.92 (m, 3H), 7.72-7.69 (m, 1H), 7.30-7.25 (m, 2H), 7.28 (t, J=8.0 Hz, 1H), 6.26-6.24 (m, 1H), 4.41-4.38 (m, 2H), 3.70 (s, 3H). 3.50-3.44 (m, 2H), 2.82 (t, J=4.0 Hz, 1H), 1.89-1.86 (m, 2H), 1.48-1.45 (m, 2H); ESI-MS(m/z): 578.1 [M+H]⁺.

### Example 72

### Preparation of 2,4-difluoro-N-(2-methoxy-5-(4-(4-(3-oxocyclohex-1-ene-1-carbonyl)piperazin-1-yl)quinazolin-6-yl)pyridin-3-yl)benzenesulfonamide

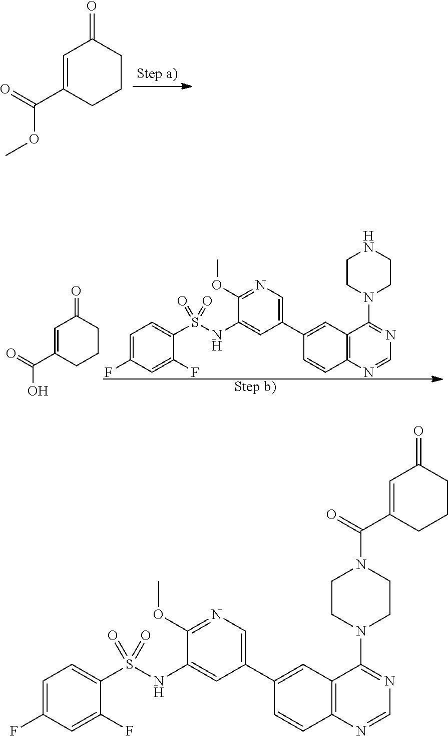

### Step a): Preparation of 3-oxocyclohex-1-ene-1-carboxylic acid

Methyl 3-oxocyclohex-1-ene-1-carboxylate (300 mg, 1.948 mmol), sodium carbonate (309.7 mg, 2.922 mmol) were dissolved in tetrahydrofuran/methanol/water (4:1:1, 6 mL). The reaction mixture was stirred at room temperature and reacted overnight. Upon completion of the reaction, the reaction was quenched by adding water (50 mL). The reaction mixture was adjusted to pH=3˜4 with hydrochloric acid (2 M) and extracted with ethyl acetate (100 mL×2 times). The organic phases were combined, washed with saturated brine (100 mL), dried over anhydrous sodium sulfate and filtered. The filtrate was concentrated under reduced pressure. 3-oxocyclohex-1-ene-1-carboxylic acid was obtained, yield: 55.1%.

### Step b): Preparation of 2,4-difluoro-N-(2-methoxy-5-(4-(4-(3-oxocyclohex-1-ene-1-carbonyl)piperazin-1-yl)quinazolin-6-yl)pyridin-3-yl)benzenesulfonamide

2,4-difluoro-N-(2-methoxy-5-(4-(piperazin-1-yl)quinazolin-6-yl)pyridin-3-yl)benzenesulfonamide (200.0 mg, 0.390 mmol) and HATU (222.2 mg, 0.585 mmol) were dissolved in dichloromethane (10 mL). The reaction system was cooled to −78° C., with N,N-diisopropylethylamine (253.4 mg, 0.975 mmol), 3-oxocyclohex-1-ene-1-carboxylic acid (81.9 mg, 1.950 mmol) added successively. After the addition was completed, the reaction mixture was kept at −78° C. and stirred for 1 h. Upon completion of the reaction, the reaction was quenched by adding water (5 mL). The reaction mixture was extracted by adding dichloromethane (10 mL/2 times). The organic phases were combined, dried over anhydrous sodium sulfate and filtered. The filtrate was concentrated under reduced pressure. The crude product obtained was purified by Prep-HPLC (Method 2), 2,4-difluoro-N-(2-methoxy-5-(4-(4-(3-oxocyclohex-1-ene-1-carbonyl)piperazin-1-yl)quinazolin-6-yl)pyridin-3-yl)benzenesulfonamide was obtained, yield: 15.0%; ¹H NMR (400 MHz, DMSO-d₆) δ 10.32 (s, 1H), 8.67 (s, 11H), 8.45 (s, 1H), 8.09-8.07 (m, 2H), 8.00 (s, 1H), 7.92 (d, J=8.0 Hz, 1H), 7.79-7.76 (m, 1H), 7.55-7.54 (m, 1H), 7.21-7.18 (m, 1H), 5.93 (s, 1H), 3.86-3.68 (m, 1H), 2.53-2.52 (m, 2H), 2.41-2.38 (m, 2H), 2.04-2.01 (m, 2H); ESI-MS(m/z): 635.0 [M+H]⁺.

### Example 73

### Preparation of 5-chloro-N-(2-methoxy-5-(4-(4-(3-oxocyclohexane-1-en-1-carbonyl)piperazin-1-yl)quinazoline-6-yl)pyridin-3-yl)thiophene-2-sulfonamide

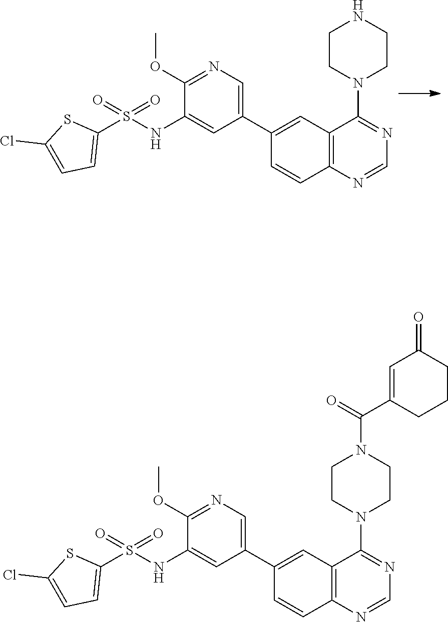 [SMILES: COc1ncc(-c2ccc3ncnc(N4CCNCC4)c3c2)cc1NS(=O)(=O)c1ccc(Cl)s1 ; COc1ncc(-c2ccc3ncnc(N4CCN(C(=O)C5=CC(=O)CCC5)CC4)c3c2)cc1NS(=O)(=O)c1ccc(Cl)s1]

Referring to the preparation method of Example 72, 5-chloro-N-(2-methoxy-5-(4-(4-(3-oxocyclohexane-1-en-1-carbonyl)piperazin-1-yl)quinazoline-6-yl)pyridin-3-yl)thiophene-2-sulfonamide was obtained, yield: 16.6%; ¹H NMR (400 MHz, DMSO-d₆) δ 10.41 (s, 1H), 8.67 (s, 1H), 8.48 (d, J=4.0 Hz, 1H), 8.11-8.09 (m, 2H), 8.01 (d, J=4.0 Hz, 1H), 7.94-7.91 (m, 1H), 7.39 (d, J=4.0 Hz, 11H), 7.21 (d, J=4.0 Hz, 1H), 5.94 (s, 1H), 3.90-3.84 (m, 4H), 3.78 (s, 3H), 3.71-3.68 (m, 4H), 3.28-3.25 (m, 2H), 2.49-2.38 (m, 2H), 2.06-1.99 (m, 2H); ESI-MS(m/z): 639.0 [M+H]⁺.

### Example 74

### Preparation of 2,4-difluoro-N-(2-methoxy-5-(4-(4-(3-oxocyclopent-1-ene-1-carbonyl)piperazin-1-yl)quinazolin-6-yl)pyridin-3-yl)benzenesulfonamide

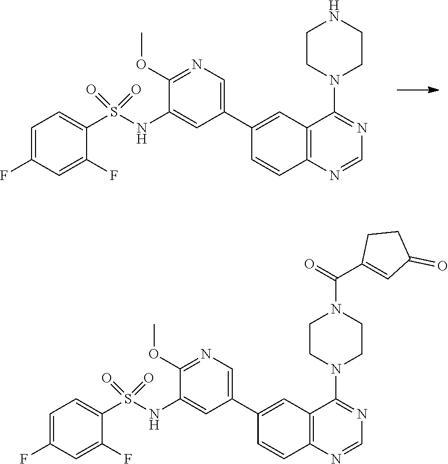 [SMILES: COc1ncc(-c2ccc3ncnc(N4CCNCC4)c3c2)cc1NS(=O)(=O)c1ccc(F)cc1F ; COc1ncc(-c2ccc3ncnc(N4CCN(C(=O)C5=CC(=O)CC5)CC4)c3c2)cc1NS(=O)(=O)c1ccc(F)cc1F]

Referring to the preparation method of Example 72, 2,4-difluoro-N-(2-methoxy-5-(4-(4-(3-oxocyclopent-1-ene-1-carbonyl)piperazin-1-yl)quinazolin-6-yl)pyridin-3-yl)benzenesulfonamide was obtained, yield: 10.6%; ¹H NMR (400 MHz, DMSO-d₆) δ 10.36 (s, 1H), 8.85 (s, 1H), 8.51 (s, 1H), 8.27-8.26 (m, 2H), 8.05 (s, 1H), 7.93 (d, J=8.0 Hz, 1H), 7.77-7.74 (m, 1H), 7.60-7.55 (m, 1H), 7.24-7.20 (m, 1H), 6.31 (s, 1H), 4.24-4.10 (m, 3H), 3.77 (s, 2H), 3.74 (s, 2H), 3.67 (s, 3H), 3.48-3.46 (m, 1H), 2.86-2.84 (m, 2H), 2.45-2.42 (m, 211); ESI-MS(m/z): 621.0 [M+H]⁺.

### Example 75

### Preparation of (E)-2,4-difluoro-N-(2-methoxy-5-(4-(4-(3-(methylsulfonyl)acryloyl)piperazin-1-yl)quinazolin-6-yl)pyridin-3-v)benzenesulfonamide

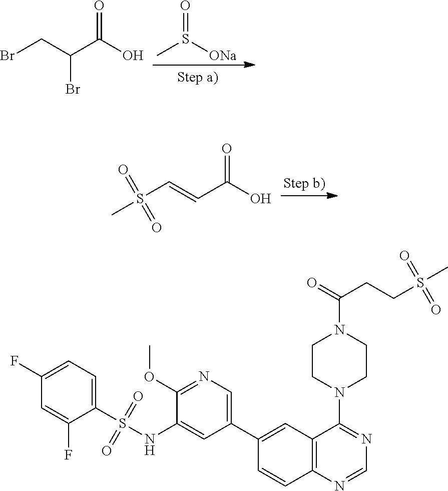

### Step a): Preparation of (E)-3-(methylsulfonyl)acrylic acid

2,3-dibromopropanoic acid (500 mg, 2.155 mmol) and sodium methanesulfinate (263.8 mg, 2.586 mmol) were dissolved in N,N-dimethylformamide/water (4:1, 10 mL). At 80 C. the reaction mixture was stirred and reacted overnight. Upon completion of the reaction, the reaction was quenched by adding water (50 mL). The reaction mixture was extracted with ethyl acetate (100 mL×2 times). The organic phases were combined, washed with saturated brine (100 mL), dried over anhydrous sodium sulfate and filtered. The filtrate was concentrated under reduced pressure. (E)-3-(methylsulfonyl)acrylic acid was obtained, yield: 61.9%. The product can be used directly in the next reaction without further purification.

### Step b): Preparation of (E)-2,4-difluoro-N-(2-methoxy-5-(4-(4-(3-(methylsulfonyl)acryloyl)piperazin-1-yl)quinazolin-6-yl)pyridin-3-yl)benzenesulfonamide

Referring to the preparation method of Example 72, Step b, (E)-2,4-difluoro-N-(2-methoxy-5-(4-(4-(3-(methylsulfonyl)acryloyl)piperazin-1-yl)quinazolin-6-yl)pyridin-3-yl)benzenesulfonamide was obtained, yield: 10.7%; ¹H NMR (400 MHz, DMSO-d₆) δ 8.63 (s, 1H), 7.92-7.78 (m, 5H), 7.56 (s, 1H), 7.55-7.37 (m, 2H), 7.20-7.18 (m, 1H), 7.09-7.05 (m, 1H), 3.85-3.79 (m, 11H), 3.17 (s, 3H); ESI-MS(m/z): 645.0 [M+H]⁺.

### Example 76

### Preparation of (E)-N-(5-(4-(4-(3-(dimethylphosphoryl)acryloyl)piperazin-1-yl)quinazolin-6-yl)-2-methoxypyridin-3-yl)-2,4-difluorobenzenesulfonamide

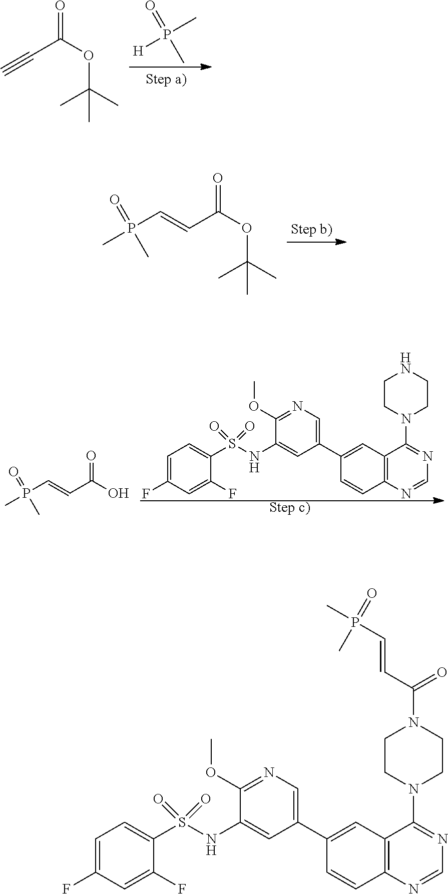

### Step a): Preparation of tert-butyl (E)-3-(dimethylphosphoryl)acrylate

Tert-butyl propiolate (1.0 g, 9.730 mmol) was dissolved in tetrahydrofuran (10 mL), with dimethylphosphine oxide (620 mg, 9.730 mmol) added. After the addition was completed, the reaction mixture was heated to 50° C. and stirred for 12 h. Upon completion of the reaction, the reaction mixture was concentrated under reduced pressure. The crude product obtained was purified by silica gel column chromatography (Eluent: petroleum ether/ethyl acetate=20/1 to 10/1). Tert-butyl (E)-3-(dimethylphosphoryl)acrylate was obtained, yield: 58.7%.

### Step b): Preparation of (E)-3-(dimethylphosphoryl)acrylic acid

Tert-butyl (E)-3-(dimethylphosphoryl)acrylate (50 mg, 0.240 mmol) was dissolved in dichloromethane (3 mL). After the addition was completed, the reaction mixture was cooled to 0° C., with TFA (1 mL) added slowly. After the addition was completed, the reaction mixture was kept at current temperature and stirred for 30 min. Upon completion of the reaction, the reaction mixture was concentrated under reduced pressure. (E)-3-(dimethylphosphoryl)acrylic acid was obtained. ESI-MS(m/z): 147 [M−H]⁺.

### Step c): Preparation of (E)-N-(5-(4-(4-(3-(dimethylphosphoryl)acryloyl)piperazin-1-yl)quinazolin-6-yl)-2-methoxypyridin-3-yl)-2,4-difluorobenzenesulfonamide

2,4-difluoro-N-(2-methoxy-5-(4-(piperazin-1-yl)quinazolin-6-yl)pyridin-3-yl)benzenesulfonamide trifluoroacetate (60 mg, 0.098 mmol) and tetrahydrofuran (2 mL) were added to the reaction flask. The reaction system was cooled to −78° C., with N,N-diisopropylethylamine (76 mg, 0.590 mmol), (E)-3-(dimethylphosphoryl)acrylic acid (15 mg, 0.098 mmol) and 50% T₃P ethyl acetate solution (62 mg, 0.098 mmol) added successively. After the addition was completed, the reaction mixture was kept at −78° C. and stirred for 1 h. Upon completion of the reaction, the reaction was quenched by adding acetonitrile (1 mL), the reaction solution was purified by Prep-HPLC (Method 2). (E)-N-(5-(4-(4-(3-(dimethylphosphoryl)acryoyl)piperazin-1-yl)quinazolin-6-yl)-2-methoxypyridin-3-yl)-2,4-difluorobenzenesulfonamide was obtained, yield: 21.9%; ¹H NMR (400 MHz, DMSO-d₆) δ 10.27 (s, 1H), 8.72 (s, 1H), 8.50 (d, J=4.0 Hz, 1H), 8.19-8.14 (m, 2H), 8.03 (s, 1H), 7.92 (d, J=8.0 Hz, 1H), 7.79-7.74 (m, 1H), 7.61-7.55 (m, 1H), 7.24-7.03 (m, 3H), 4.01 (m, 4H), 3.95-3.80 (m, 2H), 3.82-3.80 (m, 2H), 3.69 (s, 3H), 1.55 (s, 3H), 1.51 (s, 3H); ESI-MS(m/z): 643.0 [M+H]⁺.

### Example 77

### Preparation of (Z)-2,4-difluoro-N-(5-(4-(4-(2-fluoro-4-oxopent-2-enoyl)piperazin-1-yl)quinazolin-6-yl)-2-methoxypyridin-3-yl)benzenesulfonamide

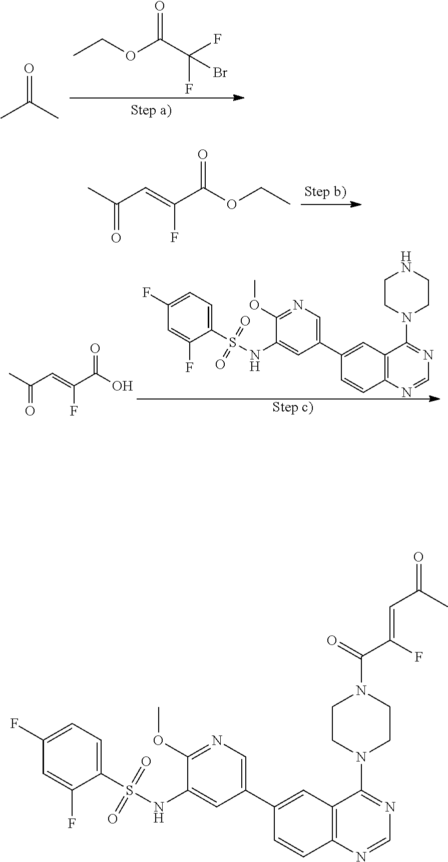

### Step a): Preparation of ethyl (Z)-2-fluoro-4-oxopent-2-enoate

Acetone (1.0 g, 17.218 mmol), cuprous iodide (328 mg, 1.722 mmol) and acetonitrile (20 mL) were added to the reaction flask. Under the ice bath condition, pentamethyldiethylenetriamine (4.48 g, 25.827 mmol), trimethylsilyl iodide (5.17 g, 25.827 mmol) and ethyl difluorobromoacetate (8.74 g, 43.045 mmol) were then added successively. After the addition was completed, the reaction mixture was kept at room temperature and reacted for 10 min, then heated to 60° C. and reacted overnight. Upon completion of the reaction, under the ice bath condition, the reaction was quenched by adding water (50 mL). The reaction mixture was extracted with ethyl acetate (100 mL×2 times). The organic phases were combined, washed with saturated brine (100 mL), dried over anhydrous sodium sulfate and filtered. The filtrate was concentrated under reduced pressure. The crude product obtained was purified by silica gel column chromatography (Eluent: ethyl acetate/petroleum ether=1/100 to 1/20). Ethyl (Z)-2-fluoro-4-oxopent-2-enoate was obtained, yield: 34.2%; ¹H NMR (400 MHz, CDCl₃) δ 6.40 (d, J=34.0 Hz, 1H), 4.36 (q, J=7.2 Hz, 2H), 2.46 (d, J=2.8 Hz, 3H), 1.37 (t, J=7.2 Hz, 3H).

### Step b): Preparation of (Z)-2-fluoro-4-oxopent-2-enoic acid

Ethyl (Z)-2-fluoro-4-oxopent-2-enoate (150 mg, 0.937 mmol) was added to tetrahydrofuran (2 mL), with sodium bicarbonate solution (2 mL, 8.4% w/w) added. After the addition was completed, the reaction mixture was heated to 70° C. and reacted for 2 h. Upon completion of the reaction, the reaction mixture was adjusted to pH=1 with dilute hydrochloric acid (2M) under the ice bath condition, extracted with ethyl acetate (50 mL/2 times). The organic phases were combined, washed with saturated brine (50 mL), dried over anhydrous sodium sulfate and filtered. The filtrate was concentrated under reduced pressure. (Z)-2-fluoro-4-oxopent-2-enoic acid was obtained, yield: 58.8%; ESI-MS(m/z): 131.0 [M−H]⁻.

### Step c): Preparation of (Z)-2,4-difluoro-N-(5-(4-(4-(2-fluoro-4-oxopent-2-enoyl)piperazin-1-yl)quinazolin-6-yl)-2-methoxypyridin-3-yl)benzenesulfonamide

Referring to the preparation method of Example 72. Step b, (Z)-2,4-difluoro-N-(5-(4-(4-(2-fluoro-4-oxopent-2-enoyl)piperazin-1-yl)quinazolin-6-yl)-2-methoxypyridin-3-yl)benzenesulfonamide was obtained, yield: 20.1%; ¹H NMR (400 MHz, DMSO-d₆) δ 10.36 (s, 1H), 8.68 (s, 1H), 8.49 (d, J=4.0 Hz, 1H), 8.16-8.08 (m, 2H), 8.03 (d, J=4.0 Hz, 1H), 7.93 (d, J=8.0 Hz, 1H), 7.83-7.72 (m, 11H), 7.63-7.53 (m, 1H), 7.27-7.17 (m, 1H), 6.12 (d, J=400 Hz, 1H), 3.95-3.79 (m, 8H), 3.68 (s, 3H), 2.37 (d, J=4.0 Hz, 3H); ESI-MS(m/z): 627.0 [M+H]⁺.

### Example 78

### Preparation of (E)-2,4-difluoro-N-(2-methoxy-5-(4-(4-(4-oxopent-2-enoyl)piperazin-1-yl)quinazolin-6-yl)pyridin-3-yl)-N-methylbenzenesulfonamide

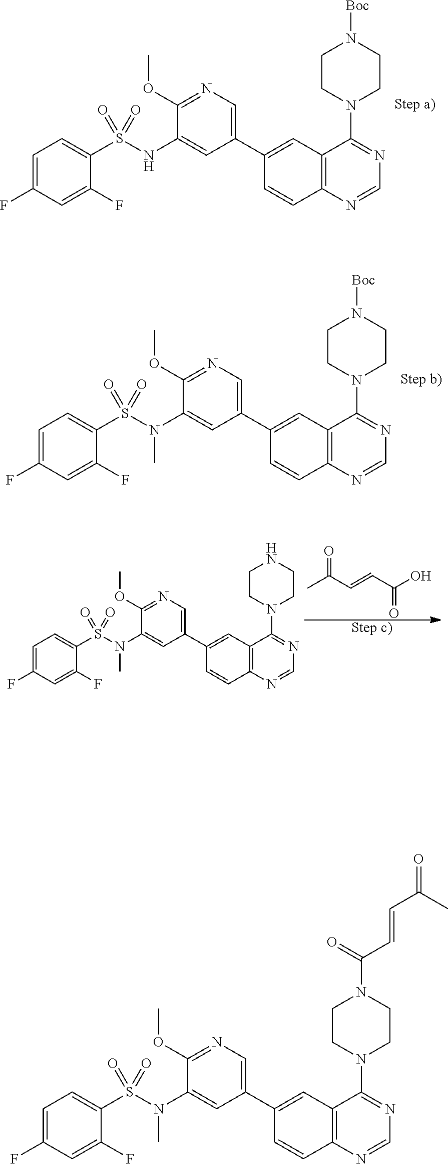

### Step a): Preparation of tert-butyl 4-(6-(5-((2,4-difluoro-N-methylphenyl)sulfonamido)-6-methoxypyridin-3-yl)quinazolin-4-yl)piperazine-1-carboxylate

Tert-butyl 4-(6-(5-((2,4-difluorophenyl)sulfonamido)-6-methoxypyridin-3-yl)quinazolin-4-yl)piperazine-1l-carboxylate (500 mg, 0.817 mmol), methyl iodide (580 mg, 4.085 mmol), cesium carbonate (1.34 g, 4.085 mmol) and N,N-dimethylformamide (10 mL) were added to the reaction flask. After the addition was completed, the reaction mixture was stirred at room temperature and reacted for 2 h. Upon completion of the reaction, the reaction was quenched by adding water (50 mL). The reaction mixture was extracted with ethyl acetate (200 mL×2 times). The organic phases were combined, washed with saturated brine (100 mL), dried over anhydrous sodium sulfate and filtered. The filtrate was concentrated under reduced pressure. The crude product obtained was purified by silica gel column chromatography (Eluent: dichloromethane/methanol=100/1 to 50/1). Tert-butyl 4-(6-(5-((2,4-difluoro-N-methylphenyl)sulfonamido)-6-methoxypyridin-3-yl)quinazolin-4-yl)piperazine-1-carboxylate was obtained, yield: 45.2%; ESI-MS(m/z): 627.2 [M+H]⁺.

### Step b): Preparation of 2,4-difluoro-N-(2-methoxy-5-(4-(piperazin-1-yl)quinazolin-6-yl)pyridin-3-yl)-N-methylbenzenesulfonamide trifluoroacetate

Tert-butyl 4-(6-(5-((2,4-difluoro-N-methylphenyl)sulfonamido)-6-methoxypyridin-3-yl)quinazolin-4-yl)piperazine-1-carboxylate (200 mg, 0.319 mmol) was dissolved in dichloromethane (3 mL). Under the ice bath condition, TFA (1 mL) was slowly added dropwise. After the addition was completed, the reaction mixture was stirred at room temperature for 1 h. Upon completion of the reaction, the reaction mixture was concentrated under reduced pressure. The crude product was slurried with methyl tert-butyl ether (5 mL) to precipitate solid and filtered. 2,4-difluoro-N-(2-methoxy-5-(4-(piperazin-1-yl)quinazolin-6-yl)pyridin-3-yl)-N-methylbenzenesulfonamide trifluoroacetate was obtained, yield: 95.6%; ESI-MS(m/z): 513.1 [M+H]⁺.

### Step c): Preparation of (E)-2,4-difluoro-N-(2-methoxy-5-(4-(4-(4-oxopent-2-enoyl)piperazin-1-yl)quinazolin-6-yl)pyridin-3-yl)-N-methylbenzenesulfonamide

2,4-difluoro-N-(2-methoxy-5-(4-(piperazin-1-yl)quinazolin-6-yl)pyridin-3-yl)-N-methylbenzenesulfonamide (85 mg, 0.133 mmol) was dissolved in tetrahydrofuran (2 mL). The reaction system was cooled to −78° C., with N,N-diisopropylethylamine (86 mg, 0.665 mmol), (E)-4-oxopent-2-enoic acid (15 mg, 0.133 mmol) and 50% T₃P ethyl acetate solution (169 mg, 0.266 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC (Method 2). (E)-2,4-difluoro-N-(2-methoxy-5-(4-(4-(4-oxopent-2-enoyl)piperazin-1-yl)quinazolin-6-yl)pyridin-3-yl)-N-methylbenzenesulfonamide was obtained, yield: 15.2%; ¹H NMR (400 MHz, DMSO-d₆+D₂O) δ 8.65 (s, 1H), 8.63 (d, J=4.0 Hz, 1H), 8.18-8.13 (m, 3H), 7.94 (d, J=12.0 Hz, 1H), 7.79-7.73 (m, 1H), 7.61-7.56 (m, 1H), 7.43 (d, J=16.0 Hz, 1H), 7.28-7.24 (m, 1H), 6.74 (d, J=16.0 Hz, 1H), 3.96-3.80 (m, 8H), 3.65 (s, 3H), 3.29 (s, 3H), 2.37 (s, 3H); ESI-MS(m/z): 623.0 [M+H]⁺.

### Example 79

### Preparation of 2,4-difluoro-N-(5-(4-(4-(2-fluoroacryloyl)piperazin-1-yl)quinazolin-6-yl)-2-methoxypyridin-3-yl)-N-methylbenzenesulfonamide

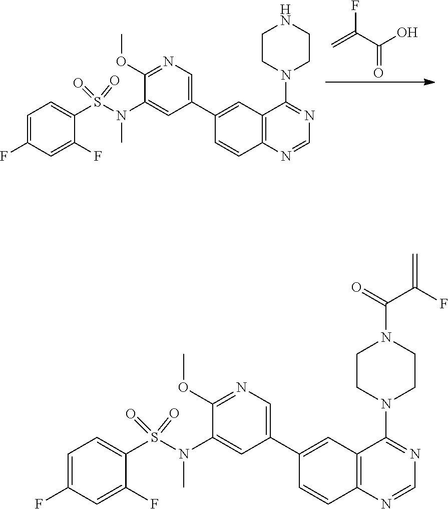

Referring to the preparation method of Example 78, Step c, 2,4-difluoro-N-(5-(4-(4-(2-fluoroacryloyl)piperazin-1-yl)quinazolin-6-yl)-2-methoxypyridin-3-yl)-N-methylbenzenesulfonamide was obtained, yield: 14.3%; ¹H NMR (400 MHz, DMSO-d₆+D₂O) δ 8.66-8.63 (m, 2H), 8.18-8.13 (m, 3H), 7.93 (d, J=8.0 Hz, 1H), 7.79-7.73 (m, 1H), 7.61-7.56 (m, 1H), 7.28-7.24 (m, 1H), 5.38-5.20 (m, 2H), 3.96-3.81 (m, 8H), 3.65 (s, 3H), 3.29 (s, 3H), 2.37 (s, 3H); ESI-MS(m/z): 623.0 [M+H]⁺.

### Example 80

### Preparation of (E)-2,4-difluoro-N-(2-methoxy-5-(4-(4-(4-oxohept-2-enoyl)piperazin-1-yl)quinazolin-6-yl)pyridin-3-yl)-N-methylbenzenesulfonamide

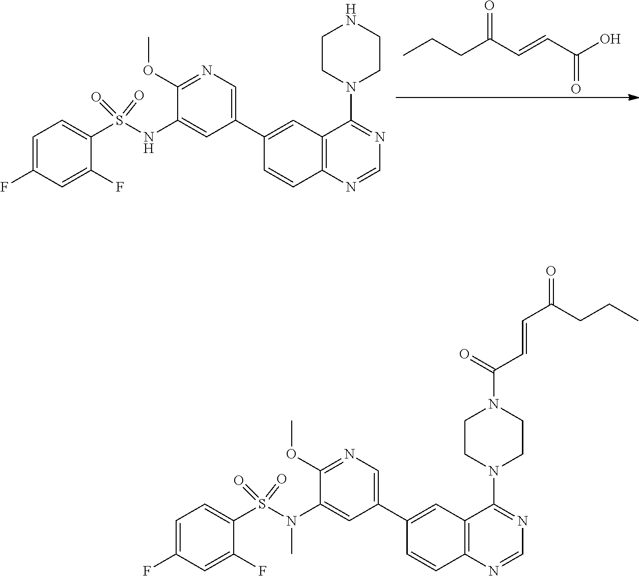

Referring to the preparation method of Example 78, Step c, (f)-2,4-difluoro-N-(2-methoxy-5-(4-(4-(4-oxohept-2-enoyl)piperazin-1-yl)quinazolin-6-yl)pyridin-3-yl)-N-methylbenzenesulfonamide was obtained, yield: 18.8%; ¹H NMR (400 MHz, DMSO-d₆) δ 8.66 (s, 1H), 8.64 (d, J=8.0 Hz, 1H), 8.18-8.13 (m, 3H), 7.92 (d, J=8.0 Hz, 1H), 7.78-7.72 (m, 1H), 7.67-7.61 (m, 1H), 7.43 (d, J=16.0 Hz, 1H), 7.28-7.23 (m, 1H), 6.82 (d, J=16.0 Hz, 1H), 3.94-3.79 (m, 8H), 3.79 (s, 3H), 3.32-3.29 (m, 3H), 2.73 (t, J=8.0 Hz, 2H), 1.60-1.51 (m, 2H), 0.89 (t, J=12.0 Hz, 3H); ESI-MS(m/z): 651.0 [M+H]⁺.

### Example 81

### Preparation of (E)-2,4-difluoro-N-(2-methoxy-5-(4-(2-(4-oxopent-2-enoyl)-2,6-diazaspiro[3.4]octan-6-yl)quinazolin-6-yl)pyridin-3-yl)-N-methylbenzenesulfonamide

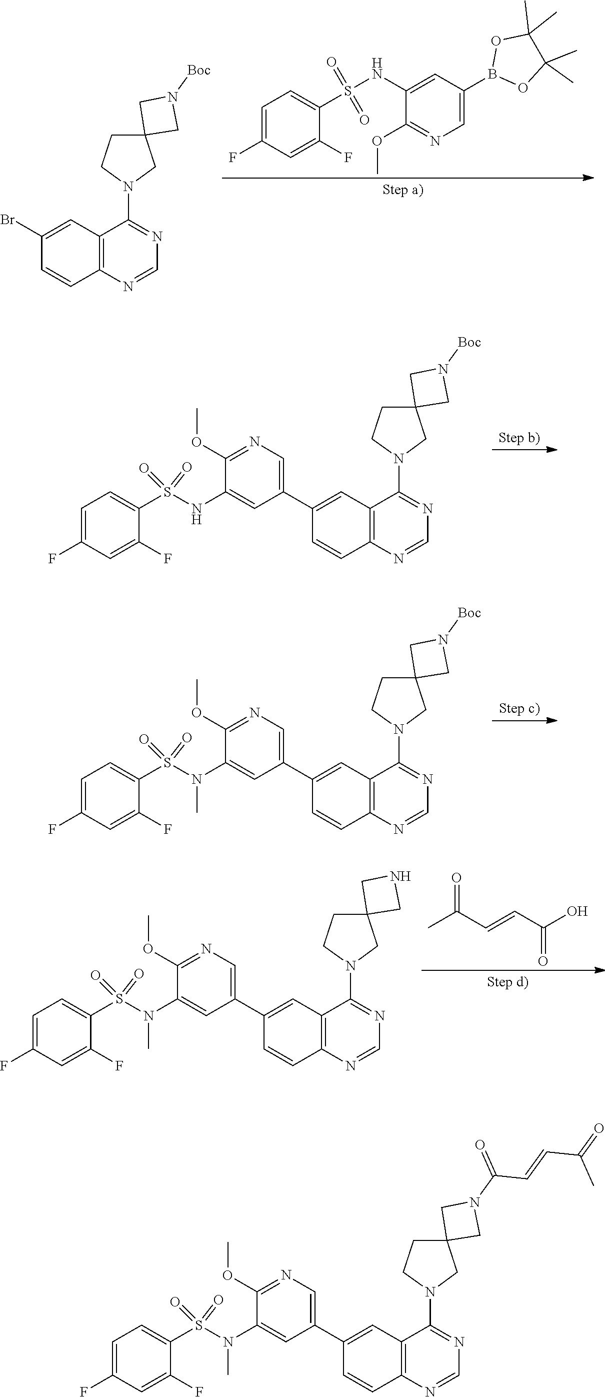

### Step a): Preparation of tert-butyl 6-(6-(5-((2,4-difluorophenyl)sulfonamido)-6-methoxypyridin-3-yl)quinazolin-4-yl)-2,6-diazaspiro[3.4]octane-2-carboxylate

Tert-butyl 6-(6-bromoquinazolin-4-yl)-2,6-diazaspiro[3.4]octane-2-carboxylate (200 mg, 0.478 mmol), 2,4-difluoro-N-(2-hydroxy-5-(4,4,5,5-tetramethyl-1,3,2-dioxaborolan-2-yl)pyridin-3-yl)benznesulfonamide (204 mg, 0.478 mmol), Pd(dppf)Cl₂ (70 mg, 0.096 mmol) and cesium carbonate (311 mg, 0.956 mmol) were added to dioxane/water mixed solvent (v/v=10:1, 4.4 mL) successively. After the addition was completed, in the presense of protective nitrogen, the reaction mixture was kept at 100° C. and stirred for 3 h. Upon completion of the reaction, the reaction mixture was filtered. The filtrate was concentrated under reduced pressure. The crude product obtained was purified by silica gel column chromatography (Eluent: petroleum ether/ethyl acetate=10/1 to 0/1). Tert-butyl 6-(6-(5-((2,4-difluorophenyl)sulfonamido)-6-methoxypyridin-3-yl)quinazolin-4-yl)-2,6-diazaspiro[3.4]octane-2-carboxylate was obtained, yield: 65.6%; ESI-MS(m/z): 639.2 [M+H]⁺.

### Step b-d): Preparation of (E)-2,4-difluoro-N-(2-methoxy-5-(4-(2-(4-oxopent-2-enoyl)-2,6-diazaspiro[3.4]octan-6-yl) quinazolin-6-yl)pyridin-3-yl)-N-methylbenzenesulfonamide

Referring to the preparation method of Example 78, Step a-c, (E)-2,4-difluoro-NV-(2-methoxy-5-(4-(2-(4-oxopent-2-enoyl)-2,6-diazaspiro[3.4]octan-6-yl) quinazolin-6-yl)pyridin-3-yl)-N-methylbenzenesulfonamide was obtained, yield: 48.4%; ¹H NMR (400 MHZ, DMSO-d₆) δ 8.65 (d, J=4.0 Hz, 1H), 8.49 (s, 1H), 8.41 (d, J=4.0 Hz, 1H), 8.15 (d, J=4.0 Hz, 1H), 8.06 (t, J=4.0 Hz, 1H), 7.81 (d, J=8.0 Hz, 1H), 7.77-7.71 (m, 1H), 7.66-7.61 (m, 1H), 7.28-7.23 (m, 1H), 6.89 (d, J=12.0 Hz, 1H), 6.73 (d, J=16.0 Hz, 1H), 4.41 (d, J=8.0 Hz, 1H), 4.32 (d, J=12.0 Hz, 1H), 4.22 (m, 2H), 4.10-3.97 (m, 4H), 3.63 (s, 3H), 3.29 (s, 3H), 2.32-2.24 (m, 5H); ESI-MS (m/z): 649.2 [M+H]⁺.

### Example 82

### Preparation of N-(5-(4-(4-acryloylpiperazin-1-yl)quinazolin-6-yl)-2-methoxypyridin-3-yl)-2,6-difluoro-N-methylbenzenesulfonamide

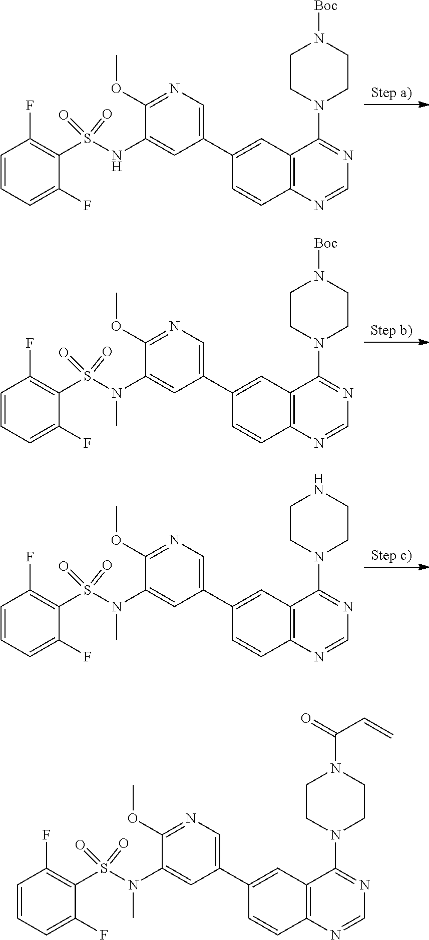

### Step a): Preparation of tert-butyl 4-(6-(5-((2,6-difluoro-N-methylphenyl)sulfonamido)-6-methoxypyridin-3-yl)quinazolin-4-yl)piperazine-1-carboxylate

Tert-butyl 4-(6-(5-((2,6-difluorophenyl)sulfonamido)-6-methoxypyridin-3-yl)quinazolin-4-yl)piperazine-1-carboxylate (240 mg, 0.392 mmol) and cesium carbonate (255 mg, 0.784 mmol) were dissolved in tert-butyl 6-(6-(5-((2,4-difluorophenyl)sulfonamido)-6-methoxypyridin-3-yl)quinazolin-4-yl)-2,6-diazaspiro[3.4]octane-2-carboxylate (8 mL), with methyl iodide (111 mg, 0.784 mmol) added. After the addition was completed, the reaction mixture was stirred at room temperature for 2 h. Upon completion of the reaction, the reaction was quenched by adding water (5 mL). The reaction mixture was extracted with ethyl acetate (100 mL×2 times). The organic phases were combined, dried over anhydrous sodium sulfate and filtered. The filtrate was concentrated under reduced pressure. The crude product obtained was purified by silica gel column chromatography (Eluent: petroleum ether/ethyl acetate=2/1 to 1/2). Tert-butyl 4-(6-(5-((2,6-difluoro-N-methylphenyl)sulfonamido)-6-methoxypyridin-3-yl)quinazolin-4-yl)piperazine-1-carboxylate was obtained, yield: 61.2%; ESI-MS(m/z): 627.1 [M+H]⁺.

### Step b): Preparation of 2,6-difluoro-N-(2-methoxy-5-(4-(piperazin-1-yl)quinazolin-6-yl)pyridin-3-yl)-N-methylbenzenesulfonamide trifluoroacetate

Tert-butyl 4-(6-(5-((2,6-difluoro-N-methylphenyl)sulfonamido)-6-methoxypyridin-3-yl)quinazolin-4-yl)piperazine-1-carboxylate (150 mg, 0.239 mmol) was dissolved in dichloromethane (4 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. 2,6-difluoro-N-(2-methoxy-5-(4-(piperazin-1-yl)quinazolin-6-yl)pyridin-3-yl)-N-methylbenzenesulfonamide trifluoroacetate was obtained; ESI-MS(m/z): 527.2 [M+H]⁺.

### Step c): Preparation of N-(5-(4-(4-acryloylpiperazin-1-yl)quinazolin-6-yl)-2-methoxypyridin-3-yl)-2,6-difluoro-N-methylbenzenesulfonamide

2,6-difluoro-N-(2-methoxy-5-(4-(piperazin-1-yl)quinazolin-6-yl)pyridin-3-yl)-N-methylbenzenesulfonamide (120 mg, 0.187 mmol) was dissolved in dichloromethane (5 mL). The reaction system was cooled to −78° C., with N,N-diisopropylethylamine (148 mg, 1.140 mmol), acrylic acid (25 mg, 0.342 mmol) and HATU (130 mg, 0.342 mmol) added successively. After the addition was completed, the reaction mixture was kept at −78° C. and stirred for 1 h. Upon completion of the reaction, the reaction was quenched by adding water (5 mL). The reaction mixture was extracted by adding dichloromethane (100 mL×2 times). The organic phases were combined, dried over anhydrous sodium sulfate and filtered. The filtrate was concentrated under reduced pressure. The crude product obtained was purified by Prep-HPLC (Method 2). N-(5-(4-(4-acryloylpiperazin-1-yl)quinazolin-6-yl)-2-methoxypyridin-3-yl)-2,6-difluoro-N-methylbenzenesulfonamide was obtained, yield: 24.9%; ¹H NMR (400 MHz, DMSO-d₆) δ 8.65 (s, 2H), 8.20-8.15 (m, 3H), 7.93-7.90 (m, 1H), 7.80-7.75 (m, 1H), 7.35-7.30 (m, 2H), 6.84-6.80 (m, 1H), 6.20-6.15 (m, 1H), 5.74 (d, J=8.0 Hz, 1H), 3.90-3.75 (m, 8H), 3.61 (s, 3H), 3.34 (s, 3H); ESI-MS(m/z): 581.0 [M+H]⁺.

### Example 83

### Preparation of (E)-N-(2-ethyl-5-(4-(4-(4-oxopent-2-enoyl)piperazin-1-yl)quinazolin-6-yl)pyridin-3-yl)-2,4-difluorobenzenesulfonamide

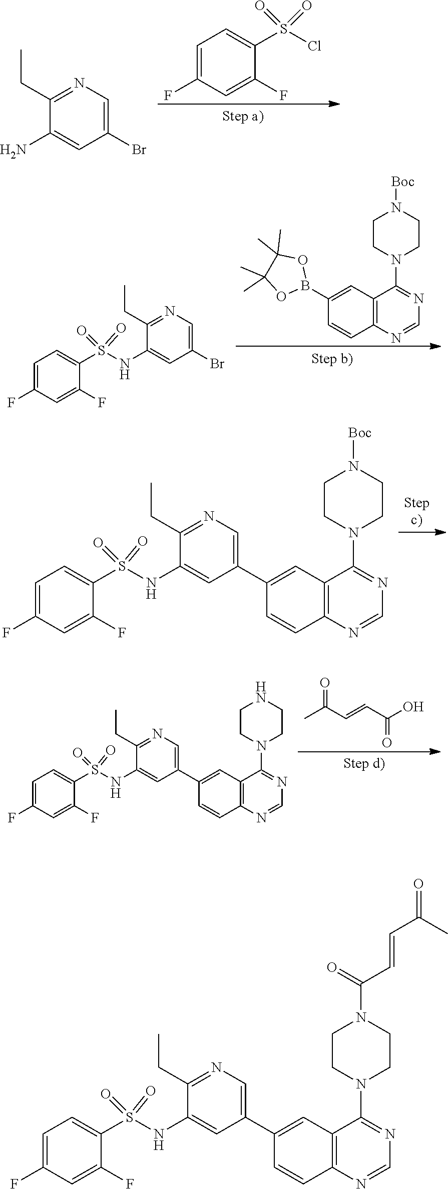

### Step a): Preparation of N-(5-bromo-2-ethylpyridin-3-yl)-2,4-difluorobenzenesulfonamide

5-Bromo-2-ethylpyridin-3-amine (550 mg, 2.736 mmol) and 2,4-difluorobenzenesulfonyl chloride (582 mg, 2.737 mmol) were dissolved in dichloromethane (6 mL), with 4-dimethylaminopyridine (33 mg, 0.270 mmol) and pyridine (433 mg, 5.474 mmol) added successively. After the addition was completed, the reaction mixture was kept at room temperature and stirred for 1 h. Upon completion of the reaction, the reaction was quenched by adding water (50 mL), and the reaction mixture was extracted with dichloromethane (100 mL/2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=5/1 to 2/1). N-(5-bromo-2-ethylpyridin-3-yl)-2,4-difluorobenzenesulfonamide was obtained, yield: 92.1%; ESI-MS(m/z): 377.0 [M+H]⁺.

### Step b): Preparation of Tert-butyl 4-(6-(5-((2,4-difluorophenyl)sulfonamido)-6-ethylpyridin-3-yl)quinazolin-4-yl)piperazine-1-carboxylate

N-(5-bromo-2-ethylpyridin-3-yl)-2,4-difluorobenzenesulfonamide (550 mg, 1.458 mmol), tert-butyl 4-(6-(4,4,5,5-tetramethyl-1,3,2-dioxaborolan-2-yl)quinazolin-4-yl)piperazine-1-carboxylate (706 mg, 1.603 mmol), Pd(dppf)Cl₂ (107 mg, 0.146 mmol) and cesium carbonate (1.4 g, 4.297 mmol) were added to dioxane/water mixed solvent (6 mL, v/v=5:1). After the addition was completed, in the presense of protective nitrogen, the reaction mixture was kept at 100° C. and stirred for 1 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=1/1 to 1/3). Tert-butyl 4-(6-(5-((2,4-difluorophenyl)sulfonamido)-6-ethylpyridin-3-yl)quinazolin-4-yl)piperazine-1-carboxylate was obtained, yield: 78.7%. ESI-MS(m/z): 611.2 [M+H]⁺.

### Step c): Preparation of N-(2-ethyl-5-(4-(piperazin-1-yl)quinazolin-6-yl)pyridin-3-yl)-2,4-difluorobenzenesulfonamide trifluoroacetate

Tert-butyl 4-(6-(5-((2,4-difluorophenyl)sulfonamido)-6-ethylpyridin-3-yl)quinazolin-4-yl)piperazine-1-carboxylate (150 mg, 0.246 mmol) was dissolved in dichloromethane (3 mL). Under the ice bath condition, TFA (1 mL) was slowly added dropwise. After the addition was completed, the reaction mixture was stirred at room temperature for 1 h. Upon completion of the reaction, the reaction mixture was concentrated under reduced pressure. Methyl tert-butyl ether was added to the crude product. The mixture was stirred to precipitate solid and filtered. N-(2-ethyl-5-(4-(piperazin-1-yl)quinazolin-6-yl)pyridin-3-yl)-2,4-difluorobenzenesulfonamide trifluoroacetate was obtained, yield: 93.8%; ESI-MS(m/z): 511.2 [M+H]⁺.

### Step d): Preparation of (E)-N-(2-ethyl-5-(4-(4-(4-oxopent-2-enoyl)piperazin-1-yl)quinazolin-6-yl)pyridin-3-yl)-2,4-difluorobenzenesulfonamide

3-Acetylacrylic acid (44 mg, 0.386 mmol) and N-(2-ethyl-5-(4-(piperazin-1-yl)quinazolin-6-yl)pyridin-3-yl)-2,4-difluorobenzenesulfonamide trifluoroacetate (120 mg, 0.192 mmol) were dissolved in THF (2 mL). The reaction system was cooled to −78° C., with N,N-diisopropylethylamine (124 mg, 0.959 mmol) and 1-propylphosphonic anhydride (367 mg, 0.576 mmol, 50% wt) added successively. After the addition was completed, the reaction mixture was kept at −78° C. and stirred for 1 h. Upon completion of the reaction, the reaction was quenched by adding water (10 mL), and the reaction mixture was extracted with dichloromethane (30 mL×2). The organic phases were combined, dried over anhydrous sodium sulfate and filtered. The filtrate was concentrated under reduced pressure. The crude product obtained was purified by Prep-HPLC (Method 2). (E)-N-(2-ethyl-5-(4-(4-(4-oxopent-2-enoyl)piperazin-1-yl)quinazolin-6-yl)pyridin-3-yl)-2,4-difluorobenzenesulfonamide was obtained, yield: 30.4%; ¹H NMR (400 MHz, DMSO-d₆) δ 10.54 (s, 1H), 8.80 (s, 1H), 8.67 (s, 1H), 8.14 (s, 1H), 8.06-8.01 (m, 1H), 7.95-7.91 (m, 1H), 7.85-7.77 (m, 2H), 7.60-7.53 (m, 1H), 7.44 (d, J=16.0 Hz, 1H), 7.27-7.20 (m, 1H), 6.74 (d, J=16.0 Hz, 1H), 3.93-3.87 (m, 6H), 3.82-3.77 (m, 2H), 2.72-2.65 (m, 2H), 2.36 (s, 3H), 1.08-1.02 (m, 3H); ESI-MS(m/z): 607.0 [M+H]⁺.

Referring to the preparation method of Example 83 and using the corresponding raw materials, the compounds in the following examples were prepared.

| Num- | | | |
|---|---|---|---|
| ber | Name | Structure | ¹H NMR and MS |
| Ex- ample 84 | (E)-N-(2- chloro-5- (4-(4-(4- oxopent-2- enoyl) piperazin-1- yl)quinazolin- 6-yl)pyridin- 3-yl)-2,6- difluoro- benzene- sulfonamide | 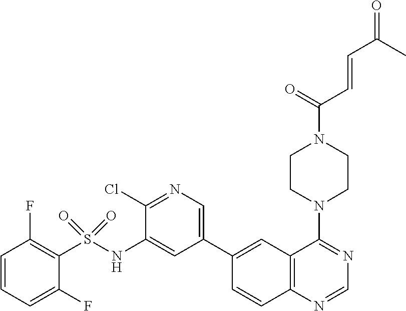 [SMILES: CC(=O)/C=C/C(=O)N1CCN(c2ncnc3ccc(-c4cnc(Cl)c(NS(=O)(=O)c5c(F)cccc5F)c4)cc23)CC1] | ¹H NMR (400 MHz, DMSO-d₆) δ 11.45 (brs, 1H), 8.70 (s, 1H), 8.60 (d, J = 4.0 Hz, 1H), 8.22-8.18 (m, 2H), 8.12 (dd, J₁ = 8.0 Hz, J₂ = 4.0 Hz, 1H), 7.94 (d, J = 8.0 Hz, 1H), 7.70- 7.63 (m, 1H), 7.45 (d, J = 16.0 Hz, 1H), 7.26-7.21 (m, 2H), 6.74 (d, J = 16.0 Hz, 1H), 3.96-3.88 (m, 6H), 3.81-3.79 (m, 2H), 2.37 (s, 3H); ESI- MS(m/z): 613.0 [M + H]⁺. |
| Ex- ample 85 | N-(2-chloro-5- (4-(4-(2- fluoroacryloyl) piperazin-1- yl)quinazolin- 6-yl)pyridin- 3-yl)-2,6- difluoro- benzene- sulfonamide | 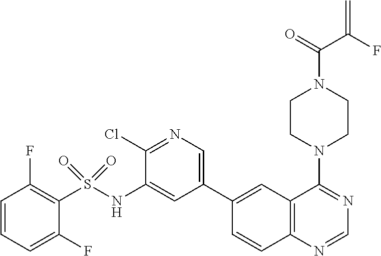 [SMILES: C=C(F)C(=O)N1CCN(c2ncnc3ccc(-c4cnc(Cl)c(NS(=O)(=O)c5c(F)cccc5F)c4)cc23)CC1] | ¹H NMR (400 MHz, DMSO-d₆) δ 11.46 (brs, 1H), 8.70 (s, 1H), 8.61 (d, J = 4.0 Hz, 1H), 8.21-8.18 (m, 2H), 8.12 (dd, J₁ = 8.0 Hz, J₂ = 4.0 Hz, 1H), 7.94 (d, J = 8.0 Hz, 1H), 7.71- 7.64 (m, 1H), 7.26-7.21 (m, 2H), 5.35-5.33 (m, 1H), 5.30 (dd, J₁ = 68.0 Hz, J₂ = 4.0 Hz, 1H), 3.96-3.94 (m, 4H), 3.87-3.77 (m, 4H); ESI- MS(m/z): 589.0 [M + H]⁺. |
| Ex- ample 86 | (E)-N-(2- chloro-5-(4-(2- (4-oxopent-2- enoyl)-2,6- diazaspiro [3.4]octan- 6-yl) quinazolin-6- yl)pyridin- 3-yl)-2,6- difluoro- benzene- sulfonamide | 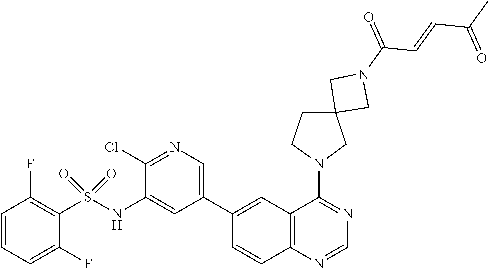 [SMILES: CC(=O)/C=C/C(=O)N1CC2(CCN(c3ncnc4ccc(-c5cnc(Cl)c(NS(=O)(=O)c6c(F)cccc6F)c5)cc34)C2)C1] | ¹H NMR (400 MHz, DMSO-d₆) δ 8.55 (s, 1H), 8.40 (s, 1H), 8.35 (s, 1H), 8.07 (d, J = 4.0 Hz, 1H), 8.01- 7.99 (m, 1H), 7.83 (d, J = 8.0 Hz, 1H), 7.60-7.52 (m, 1H), 7.27-7.22 (m, 2H), 6.88 (d, J = 16.0 Hz, 1H), 6.71 (d, J = 16.0 Hz, 1H), 4.41 (d, J = 8.0 Hz, 1H), 4.31 (d, J = 8.0 Hz, 1H), 4.24-4.17 (m, 2H), 4.10- 4.07 (m, 1H), 4.03-3.97 (m, 3H), 2.32 (s, 3H), 2.28-2.24 (m, 2H); ESI-MS(m/z): 639.0 [M + H]⁺. |
| Ex- ample 87 | (E)-N-(2- chloro-5-(4-(2- (4-oxopent-2- enoyl)-2,7- diazaspiro [3.5]nonan-7- yl)quinazolin- 6-yl)pyridin- 3-yl)-2,6- difluoro- benzene- sulfonamide | 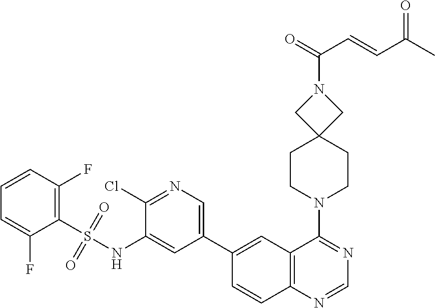 [SMILES: CC(=O)/C=C/C(=O)N1CC2(CCN(c3ncnc4ccc(-c5cnc(Cl)c(NS(=O)(=O)c6c(F)cccc6F)c5)cc34)CC2)C1] | ¹H NMR (400 MHz, DMSO-d₆) δ 11.62 (s, 1H), 8.68 (s, 1H), 8.59 (s, 1H), 8.20-8.08 (m, 3H), 7.92 (d, J = 8.8 Hz, 1H), 7.74-7.64 (m, 1H), 7.29- 7.18 (m, 2H), 6.94 (d, J = 16.0 Hz, 1H), 6.74 (d, J = 16.0 Hz, 1H), 4.15 (s, 2H), 3.89-3.72 (m, 6H), 2.35 (s, 3H), 1.97 (t, J = 5.6 Hz, 4H); ESI- MS(m/z): 653.0 [M + H]⁺. |
| Ex- ample 88 | (S,E)-N-(2- chloro-5-(4- (2-methyl-4- (4-oxopent-2- enoyl) piperazin-1- yl)quinazolin- 6-yl)pyridin- 3-yl)-2,6- difluoro- benzene- sulfonamide | 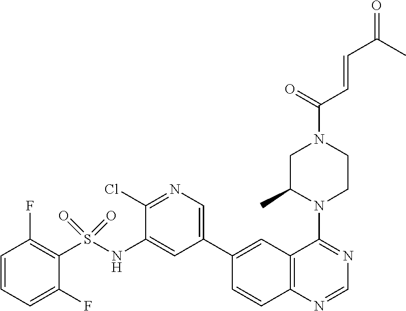 [SMILES: CC(=O)/C=C/C(=O)N1CCN(c2ncnc3ccc(-c4cnc(Cl)c(NS(=O)(=O)c5c(F)cccc5F)c4)cc23)[C@@H](C)C1] | ¹H NMR (400 MHz, DMSO-d₆) δ 8.65 (s, 1H), 8.51 (s, 1H), 8.13-8.07 (m, 3H), 7.94-7.91 (m, 1H), 7.67-7.58 (m, 1H), 7.48-7.32 (m, 1H), 7.18 (t, J = 12.0 Hz, 2H), 6.73 (dd, J₁ = 16.0 Hz, J₂ = 12.0 Hz, 1H), 4.86-4.76 (m, 1H), 4.41-4.10 (m, 3H), 4.02-3.88 (m, 1H), 3.53-3.44 (m, 1H), 3.23-3.10 (m, 1H), 2.36-2.33 (m, 3H), 1.31 (t, J = 4.0 Hz, 3H); ESI-MS(m/z): 627.0 [M + H]⁺. |
| Ex- ample 89 | (E)-N-(2- chloro-5-(4- (4-(4-oxopent- 2-enoyl) piperazin-1-yl) quinazolin-6- yl)pyridin-3- yl)-2,4,6- trifluoro- benzene- sulfonamide | 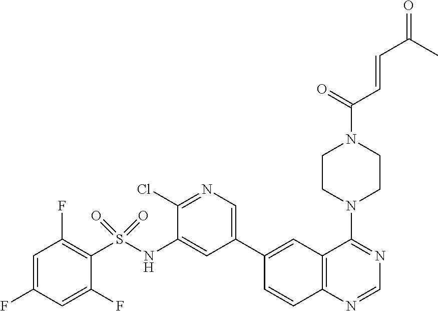 [SMILES: CC(=O)/C=C/C(=O)N1CCN(c2ncnc3ccc(-c4cnc(Cl)c(NS(=O)(=O)c5c(F)cc(F)cc5F)c4)cc23)CC1] | ¹H NMR (400 MHz, DMSO-d₆) δ 11.65 (s, 1H), 8.71 (s, 1H), 8.65-8.56 (m, 1H), 8.26-8.10 (m, 3H), 7.95 (d, J = 8.4 Hz, 1H), 7.49-7.34 (m, 3H), 6.74 (d, J = 15.6 Hz, 1H), 4.01-3.77 (m, 8H), 2.37 (s, 3H); ESI-MS(m/z): 631.0 [M + H]⁺. |
| Ex- ample 90 | (E)-2,3- difluoro-N- (2-methoxy- 5-(4-(4-(4- oxopent-2- enoyl) piperazin-1- yl)quinazolin- 6-yl)pyridin- 3-yl) benzene- sulfonamide | 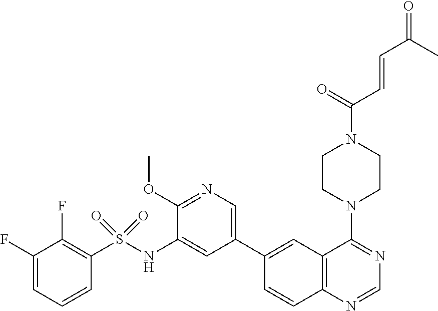 [SMILES: COc1ncc(-c2ccc3ncnc(N4CCN(C(=O)/C=C/C(C)=O)CC4)c3c2)cc1NS(=O)(=O)c1cccc(F)c1F] | ¹H NMR (400 MHz, DMSO-d₆) δ 10.50 (brs, 1H), 8.66 (s, 1H), 8.39 (s, 1H), 8.11-8.06 (m, 2H), 7.97 (d, J = 8.0 Hz, 1H), 7.91 (d, J = 8.0 Hz, 1H), 7.75-7.68 (m, 1H), 7.54-7.51 (m, 1H), 7.45 (d, J = 16.0 Hz, 1H), 7.35-7.30 (m, 1H), 6.74 (d, J = 16.0 Hz, 1H), 3.89-3.79 (m, 8H), 3.67 (s, 3H), 2.37 (s, 3H); ESI-MS(m/z): 609.0 [M + H]⁺. |
| Ex- ample 91 | (E)-2-fluoro- N-(2-methoxy- 5-(4-(4-(4- oxopent-2- enoyl) piperazin-1- yl)quinazolin- 6-yl)pyridin- 3-yl) benzene- sulfonamide | 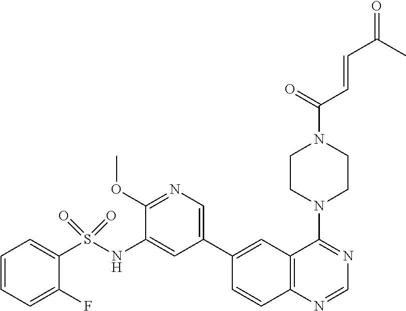 [SMILES: COc1ncc(-c2ccc3ncnc(N4CCN(C(=O)/C=C/C(C)=O)CC4)c3c2)cc1NS(=O)(=O)c1ccccc1F] | ¹H NMR (400 MHz, DMSO-d₆) δ 10.28 (s, 1H), 8.67 (s, 1H), 8.45 (d, J = 4.0 Hz, 1H), 8.12-8.06 (m, 2H), 7.99 (d, J = 8.0 Hz, 1H), 7.92 (d, J = 8.0 Hz, 1H), 7.74-7.68 (m, 2H), 7.48- 7.46 (s, 2H), 7.34-7.30 (m, 1H), 6.74 (d, J = 16.0 Hz, 1H), 3.90-3.88 (m, 6H), 3.81-3.80 (m, 2H), 3.65 (s, 3H), 2.37 (s, 3H); ESI-MS(m/z): 591.0 [M + H]⁺. |
| Ex- ample 92 | (E)-4-fluoro- N-(2-methoxy- 5-(4-(4-(4- oxopent-2- enoyl) piperazin- 1-yl) quinazolin-6- yl)pyridin-3- yl)benzene- sulfonamide | 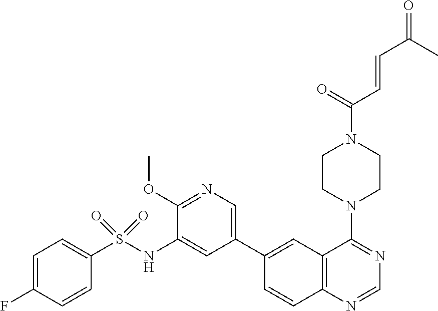 [SMILES: COc1ncc(-c2ccc3ncnc(N4CCN(C(=O)/C=C/C(C)=O)CC4)c3c2)cc1NS(=O)(=O)c1ccc(F)cc1] | ¹H NMR (400 MHz, DMSO-d₆) δ 10.11 (s, 1H), 8.67 (s, 1H), 8.45-8.43 (m, 1H), 8.12-8.11 (m, 1H), 8.10-8.06 (m, 1H), 8.00-7.98 (m, 1H), 7.92 (d, J = 8.0 Hz, 1H), 7.85-7.83 (m, 1H), 7.83-7.80 (m, 1H), 7.48-7.43 (m, 1H), 7.43-7.39 (m, 2H), 6.74 (d, J = 16.0 Hz, 1H), 3.91-3.88 (m, 6H), 3.82-3.79 (m, 2H), 3.68 (s, 3H), 2.37 (s, 3H); ESI-MS(m/z): 591.0 [M + H]⁺. |
| Ex- ample 93 | (E)-N-(2- methoxy-5-(4- (4-(4-oxopent- 2-enoyl) piperazin-1-yl) quinazolin-6- yl)pyridin-3- yl)-2-methyl- benzene- sulfonamide | 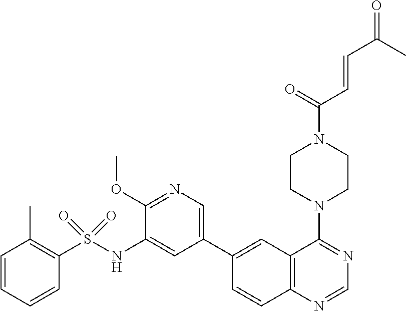 [SMILES: COc1ncc(-c2ccc3ncnc(N4CCN(C(=O)/C=C/C(C)=O)CC4)c3c2)cc1NS(=O)(=O)c1ccccc1C] | ¹HNMR (400 MHz, DMSO-d₆) δ 10.11 (s, 1H), 8.67 (s, 1H), 8.40-8.39 (m, 1H), 8.07-8.01 (m, 2H), 7.92-7.90 (m, 2H), 7.72 (d, J = 8.0 Hz, 1H), 7.75-7.41 (m, 4H), 6.74 (d, J = 16.0 Hz, 1H), 3.89 (s, 6H), 3.82-3.81 (m, 2H), 3.71 (s, 3H), 2.67 (s, 3H), 2.37 (s, 3H); ESI-MS(m/z): 587.0 [M + H]⁺. |
| Ex- ample 94 | (E)-2,5- difluoro-N- (2-methoxy- 5-(4-(4-(4- oxopent-2- enoyl) piperazin-1- yl)quinazolin- 6-yl)pyridin- 3-yl) benzene- sulfonamide | 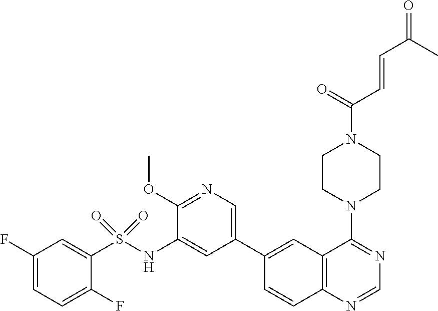 [SMILES: COc1ncc(-c2ccc3ncnc(N4CCN(C(=O)/C=C/C(C)=O)CC4)c3c2)cc1NS(=O)(=O)c1cc(F)ccc1F] | ¹H NMR (400 MHz, DMSO-d₆) δ 10.46 (s, 1H), 8.66 (s, 1H), 8.47 (s, 1H), 8.14-8.08 (m, 2H), 8.03-8.01 (m, 1H), 7.92 (d, J = 12.0 Hz, 1H), 7.59- 7.51 (m, 3H), 7.45 (d, J = 16.0 Hz, 1H), 6.74 (d, J = 16.0 Hz, 1H), 3.92- 3.87 (m, 6H), 3.82-3.78 (m, 2H), 3.68 (s, 3H), 2.37 (s, 3H); ESI-MS(m/z): 609.0 [M + H]⁺. |
| Ex- ample 95 | (E)-2,4- difluoro-N- (2-methoxy- 5-(4-(4-(4- oxopent-2- enoyl) piperazin-1- yl)quinazolin- 6-yl)phenyl) benzene- sulfonamide | 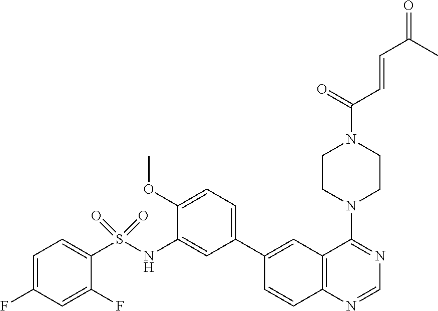 [SMILES: COc1ccc(-c2ccc3ncnc(N4CCN(C(=O)/C=C/C(C)=O)CC4)c3c2)cc1NS(=O)(=O)c1ccc(F)cc1F] | ¹H NMR (400 MHz, DMSO-d₆) δ 9.99 (s, 1H), 8.67 (s, 1H), 8.08-8.06 (m, 2H), 7.92-7.90 (m, 1H), 7.76-7.70 (m, 1H), 7.68-7.64 (m, 2H), 7.59-7.53 (m, 1H), 7.47 (d, J = 16.0 Hz, 1H), 7.22-7.15 (m, 1H), 7.07 (d, J = 8.0 Hz, 1H), 6.75 (d, J = 16.0 Hz, 1H), 4.07-3.81 (m, 8H), 3.56 (s, 3H), 2.38 (s, 3H); ESI-MS(m/z): 608.0 [M + H]⁺. |

### Example 96

### Preparation of (E)-8-chloro-N-(2-methoxy-5-(4-(4-(4-oxopent-2-enoyl)piperazin-1-yl)quinazolin-6-yl)pyridin-3-yl)naphthalene-1-sulfonamide

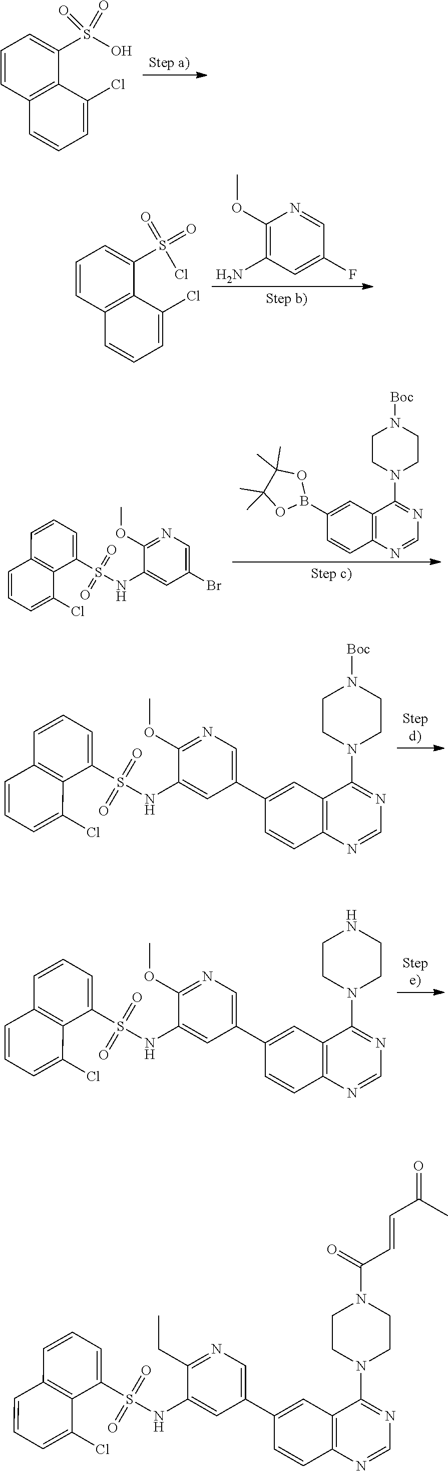

### Step a): Preparation of 8-chloronaphthalene-1-sulfonyl chloride

8-chloronaphthalene-1-sulfonic acid (200 mg, 0.820 mmol) was dissolved in dichloromethane (4 mL), with DMF (0.6 mg, 0.008 mmol) added. After the addition was completed, the reaction mixture was cooled to 0° C., with oxalyl chloride (210 mg, 1.640 mmol) added dropwise. After the addition was completed, the reaction mixture was heated to 40° C. and stirred for 3 h. Upon completion of the reaction, the reaction mixture was concentrated under reduced pressure. 8-chloronaphthalene-1-sulfonyl chloride was obtained, which can be used directly in the next reaction.

### Step b-e): Preparation of (E)-8-chloro-N-(2-methoxy-5-(4-(4-(4-oxopent-2-enoyl)piperazin-1-yl)quinazolin-6-ylpyridin-3-yl)naphthalene-1-sulfonamide

Referring to the preparation method of Example 83. (E)-8-chloro-N-(2-methoxy-5-(4-(4-(4-oxopent-2-enoyl)pyridin-1-yl)quinazolin-6-yl)pyridin-3-yl)naphthalene-1-sulfonamide was obtained, yield: 20.9%; ¹H NMR (400 MHz, DMSO-d₆) δ 10.13 (s, 1H), 8.64 (s, 1H), 8.45 (d, J=8.0 Hz, 1H), 8.39 (s, 1H), 8.29 (d, J=8.0 Hz, 1H), 8.11 (d, J=8.0 Hz, 1H), 7.95-7.85 (m, 4H), 7.76 (s, 1H), 7.70-7.62 (m, 2H), 7.45 (d, 0.1=16.0 Hz, 1H), 6.77 (d, J=16.0 Hz, 1H), 3.86-3.71 (m, 11H), 2.38 (s, 3H); ESI-MS(m/z): 657.0 [M+H]⁺.

### Example 97

### Preparation of (E)-2,4,6-trifluoro-N-(2-methoxy-5-(4-(2-(4-oxopent-2-enoyl)-2,6-diazaspiro[3.4]octan-6-yl)quinazolin-6-yl)pyridin-3-yl)benzenesulfonamide

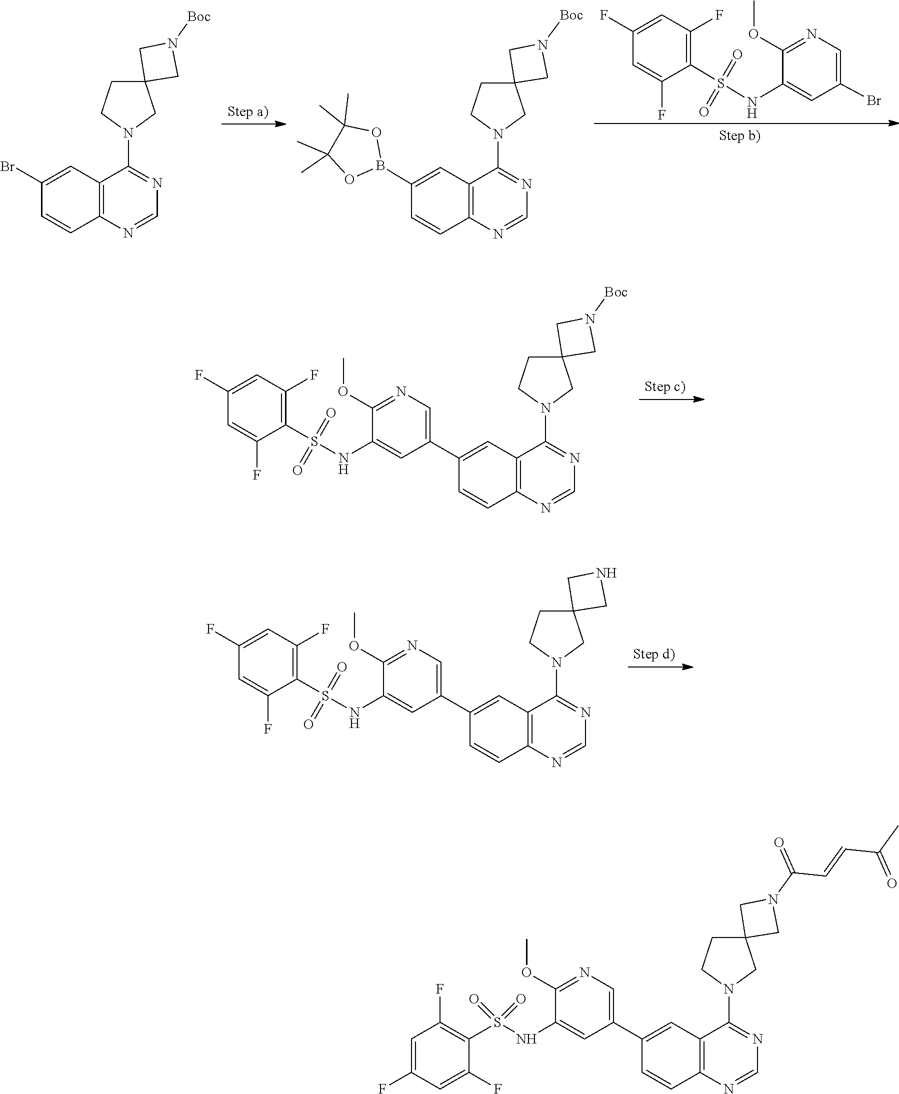

### Step a): Preparation of tert-butyl 6-(6-(4,4,5,5-tetramethyl-1,3,2-dioxaborolan-2-yl)quinazolin-4-yl)-2,6-diazaspiro[3.4]octane-2-carboxylate

Tert-butyl 6-(6-bromoquinazolin-4-yl)-2,6-diazaspiro[3.4]octane-2-carboxylate (725 mg, 2.864 mmol), Pd(dppf)Cl₂ (174 mg, 0.239 mmol) and potassium acetate (701 mg, 7.161 mmol) were added to dioxane (20 mL). After the addition was completed, in the presense of protective nitrogen, the reaction mixture was heated to 10° C. and stirred for 2 h. Upon completion of the reaction, the reaction was quenched by adding water (50 mL). The reaction mixture was extracted with ethyl acetate (150 mL×2 times). The organic phases were combined, washed with saturated brine (100 mL×1 time), dried over anhydrous sodium sulfate and filtered. The filtrate was concentrated under reduced pressure. The crude product obtained was purified by silica gel column chromatography (Eluent: dichloromethane/methanol=100/1 to 10/1). Tert-butyl 6-(6-(4,4,5,5-tetramethyl-1,3,2-dioxaborolan-2-yl)quinazolin-4-yl)-2,6-diazaspiro[3.4]octane-2-carboxylate was obtained, yield: 81.1%; ESI-MS(m/z): 467.0 [M+H]⁺.

### Step b): Preparation of tert-butyl 6-(6-(6-methoxy-5-((2,4,6-trifluorophenyl)sulfonamido)pyridin-3-yl)quinazolin-4-yl)-2,6-diazaspiro[3.4]octane-2-carboxylate

N-(5-bromo-2-methoxypyridin-3-yl)-2,4,6-trifluorobenzenesulfonamide (300 mg, 0.756 mmol), tert-butyl 6-(6-(4,4,5,5-tetramethyl-1,3,2-dioxaborolan-2-yl)quinazolin-4-yl)-2,6-diazaspiro[3.4]octane-2-carboxylate (425 mg, 0.907 mmol), Pd(dppf)Cl₂ (56 mg, 0.076 mmol) and cesium carbonate (743 g, 2.268 mmol) were added to dioxane/water mixed solvent (10 mL, v/v=4:1). After the addition was completed, in the presense of protective nitrogen, the reaction mixture was heated to 110° C. and stirred for 2 h. Upon completion of the reaction, the reaction was quenched by adding water (50 mL). The reaction mixture was extracted with ethyl acetate (150 mL×2 times). The organic phases were combined, washed with saturated brine (100 mL×1 time), dried over anhydrous sodium sulfate and filtered. The filtrate was concentrated under reduced pressure. The crude product obtained was purified by silica gel column chromatography (Eluent: dichloromethane/methanol=100/1 to 10/1).

Tert-butyl 6-(6-(6-methoxy-5-((2,4,6-trifluorophenyl)sulfonamido)pyridin-3-yl)quinazolin-4-yl)-2,6-diazaspiro[3.4]octane-2-carboxylate was obtained, yield: 50.4%. ESI-MS(m/z): 657.6 [M+H]⁺.

### Step c): Preparation of N-(5-(4-(2,6-diazaspiro[3.4]octan-6-yl)quinazolin-6-yl)-2-methoxypyridin-3-yl)-2,4,6-trifluorobenzenesulfonamide

Tert-butyl 6-(6-(6-methoxy-5-((2,4,6-trifluorophenyl)sulfonamido)pyridin-3-yl)quinazolin-4-yl)-2,6-diazaspiro[3.4]octane-2-carboxylate (250 mg, 0.381 mmol) was dissolved in dichloromethane (8 mL). Under the ice bath condition. TFA (2 mL) was slowly added dropwise. After the addition was completed, the reaction mixture was stirred at room temperature for 1 h. Upon completion of the reaction, the solvent was directly spinned dry. The product was added to 5 mL methyl tert-butyl ether to slurry, filtered. The solid was collected to obtain N-(5-(4-(2,6-diazaspiro[3.4]octan-6-yl)quinazolin-6-yl)-2-methoxypyridin-3-yl)-2,4,6-trifluorobenzenesulfonamide. ESI-MS(m/z): 557.2 [M+H]⁺.

### Step d): Preparation of (E)-2,4,6-trifluoro-N-(2-methoxy-5-(4-(2-(4-oxopent-2-enoyl)-2,6-diazaspiro[3.4]octan-6-yl)quinazolin-6-yl)pyridin-3-yl)benzenesulfonamide

N-(5-(4-(2,6-diazaspiro[3.4]octan-6-yl)quinazolin-6-yl)-2-methoxypyridin-3-yl)-2,4,6-trifluorobenzenesulfonamide (100 mg, 0.180 mmol) and (E)-4-oxopent-2-enoic acid (31 mg, 0.270 mmol) were dissolved in tetrahydrofuran (2 mL). The reaction system was cooled to −78° C., with N,N-diisopropylethylamine (116 mg, 0.900 mmol) and 1-propylphosphonic anhydride (115 mg, 0.360 mmol) added successively. After the addition was completed, the reaction mixture was kept at −78° C. and stirred for 1 h. Upon completion of the reaction, with acetonitrile (2 ml) added, the reaction solution was purified by Prep-HPLC (Method 2). (E)-2,4,6-trifluoro-N-(2-methoxy-5-(4-(2-(4-oxopent-2-enoyl)-2,6-diazaspiro[3.4]octan-6-yl)quinazolin-6-yl)pyridin-3-yl)benzenesulfonamide was obtained, yield: 20.4%; ¹HNMR (400 MHz, DMSO-d₆) δ 10.66 (s, 1H), 8.48 (s, 1H), 8.47-8.34 (m, 2H), 7.98-7.91 (m, 2H), 7.80 (d, J=12 Hz, 1H), 7.30-7.25 (m, 2H), 6.88 (d, J=16 Hz, 1H), 6.71 (d, 0.1=16 Hz, 1H), 4.43-4.41 (m, 1H), 4.32-4.30 (m, 1H), 4.21-4.19 (m, 2H), 4.11-4.08 (m, 1H), 4.05-3.96 (m, 3H), 3.71 (s, 3H), 2.32 (s, 3H), 2.28-2.24 (m, 2H); ESI-MS(m/z): 653.0 [M+H]⁺.

Referring to the preparation method of Example 97 and using the corresponding raw materials, the compounds in the following examples were prepared.

| Num- | | | |
|---|---|---|---|
| ber | Name | Structure | ¹H NMR and MS |
| Ex- ample 98 | 2,4,6-trifluoro- N-(5-(4-(4-(2- fluoroacryloyl) piperazin-1- yl)quinazolin- 6-yl)-2- methoxypyridin- 3-yl) benzene- sulfonamide | 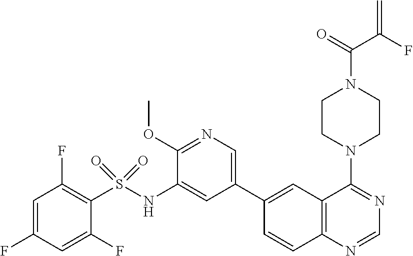 [SMILES: C=C(F)C(=O)N1CCN(c2ncnc3ccc(-c4cnc(OC)c(NS(=O)(=O)c5c(F)cc(F)cc5F)c4)cc23)CC1] | ¹H NMR (400 MHz, DMSO-d₆) δ 10.66 (s, 1H), 8.67 (s, 1H), 8.50 (s, 1H), 8.17-8.08 (m, 2H), 8.04 (d, J = 2.0 Hz, 1H), 7.92 (d, J = 8.8 Hz, 1H), 7.41 (t, J = 9.2 Hz, 2H), 5.33 (dd, J₁ = 4.0, J₂ = 2.0 Hz, 1H), 5.29 (dd, J₁ = 66.0 Hz, J₂ = 4.0 Hz, 1H), 3.96-3.86 (m, 4H), 3.80-3.78 (m, 4H), 3.69 (s, 3H); ESI-MS(m/z): 603.0 [M + H]⁺. |
| Ex- ample 99 | (E)-2,4,6- trifluoro- N-(2-methoxy- 5-(4-(4-(4- oxopent-2- enoyl) piperazin-1-yl) quinazolin-6- yl)pyridin-3-yl) benzene- sulfonamide | 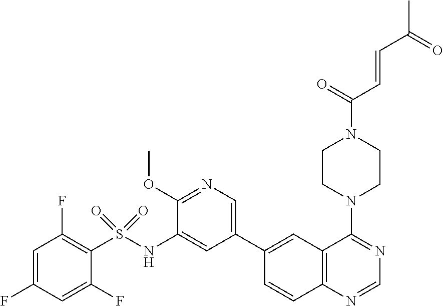 [SMILES: COc1ncc(-c2ccc3ncnc(N4CCN(C(=O)/C=C/C(C)=O)CC4)c3c2)cc1NS(=O)(=O)c1c(F)cc(F)cc1F] | ¹H NMR (400 MHz, DMSO-d₆) δ 10.66 (s, 1H), 8.67 (s, 1H), 8.54-8.44 (m, 1H), 8.16-8.08 (m, 2H), 8.05 (d, J = 2.4 Hz, 1H), 7.92 (d, J = 8.8 Hz, 1H), 7.50-7.37 (m, 3H), 6.74 (d, J = 15.6 Hz, 1H), 3.90-3.88 (m, 6H), 3.82-3.79 (m, 2H), 3.68 (s, 3H), 2.37 (s, 3H); ESI-MS(m/z): 627.0 [M + H]⁺. |
| Ex- ample 100 | N-(5-(4-(4- acryloyl- piperazin- 1-yl)quinazolin- 6-yl)-2- methoxypyridin- 3-yl)-2,4,6- trifluoro- benzene- sulfonamide | 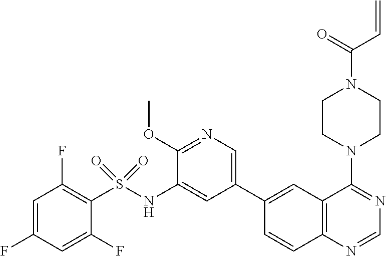 [SMILES: C=CC(=O)N1CCN(c2ncnc3ccc(-c4cnc(OC)c(NS(=O)(=O)c5c(F)cc(F)cc5F)c4)cc23)CC1] | ¹H NMR (400 MHz, DMSO-d₆) δ 10.67 (s, 1H), 8.66 (s, 1H), 8.51 (d, J = 2.4 Hz, 1H), 8.19-8.08 (m, 2H), 8.05 (d, J = 2.4 Hz, 1H), 7.91 (d, J = 8.8 Hz, 1H), 7.54-7.34 (m, 2H), 6.83 (dd, J = 16.8, 10.4 Hz, 1H), 6.17 (dd, J = 16.8, 2.4 Hz, 1H), 5.74 (dd, J = 10.4, 2.4 Hz, 1H), 3.96-3.72 (m, 8H), 3.68 (s, 3H); ESI-MS(m/z): 585.0 [M + H]⁺. |
| Ex- ample 101 | (E)-2,4,6- trifluoro-N- (2-methoxy- 5-(4- (2-(4-oxopent-2- enoyl)-2,7- diazaspiro[3.5] nonan-7-yl) quinazolin-6- yl)pyridin-3-yl) benzene- sulfonamide | 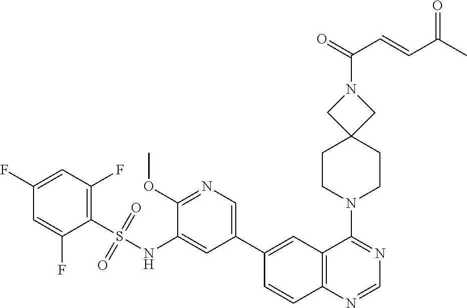 [SMILES: COc1ncc(-c2ccc3ncnc(N4CCC5(CC4)CN(C(=O)/C=C/C(C)=O)C5)c3c2)cc1NS(=O)(=O)c1c(F)cc(F)cc1F] | ¹H NMR (400 MHz, DMSO-d₆) δ 10.68 (s, 1H), 8.64 (s, 1H), 8.46 (s, 1H), 8.12-8.00 (m, 3H), 7.90 (d, J = 8.4 Hz, 1H), 7.43 (t, J = 9.2 Hz, 2H), 6.93 (d, J = 15.6 Hz, 1H), 6.74 (d, J = 15.6 Hz, 1H), 4.15 (s, 2H), 3.83-3.71 (m, 6H), 3.69 (s, 3H), 2.35 (s, 3H), 1.98-1.95 (m, 4H); ESI-MS(m/z): 667.0 [M + H]⁺. |

### Example 102

### Preparation of (E)-3,5-difluoro-N-(2-methoxy-5-(4-(4-(4-oxopent-2-enoyl)piperazin-1-yl)quinazolin-6-yl)pyridin-3-yl)pyridine-4-sulfonamide

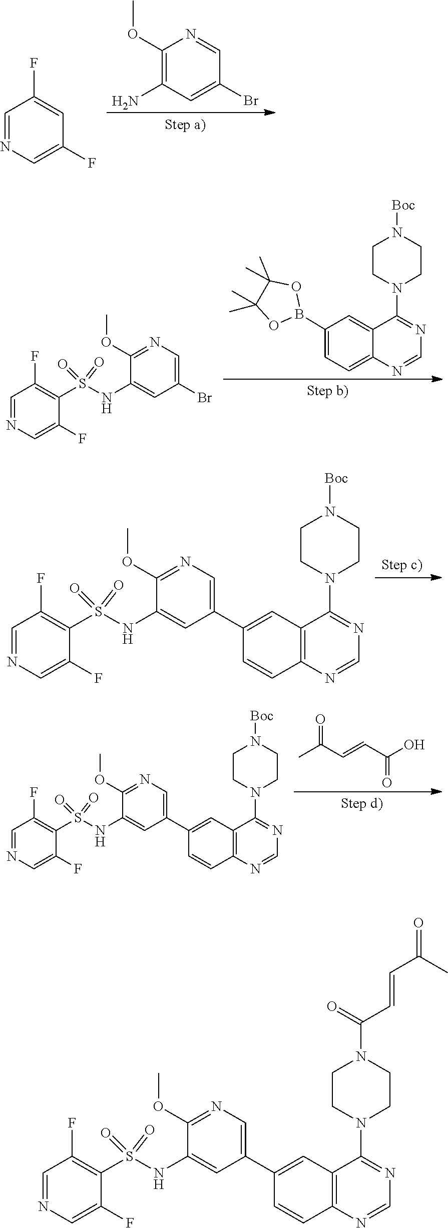

### Step a): Preparation of N-(5-bromo-2-methoxypyridin-3-yl)-3,5-difluoropyridine-4-sulfonamide

3,5-difluoropyridine (500 mg, 4.345 mmol) was added to tetrahydrofuran (20 mL), in the presense of protective nitrogen, the reaction solution was cooled to −78° C., with butyllithium solution (1.54 g, 18% wt) added. After the addition was completed, the reaction mixture was kept at −78° C. and reacted for 1 h. Then at −78° C., sulfur dioxide gas was bubble into the reaction solution for 10 min. After that, the reaction mixture was heated to room temperature and reacted for 1 h. Upon completion of the reaction, the reaction mixture was concentrated under reduced pressure. The residue was dissolved in dichloromethane (20 mL), with N-chlorosuccinimide (2.35 g, 17.600 mmoL) added, at room temperature, reacted for 2 h. Upon completion of the reaction, under the ice bath condition, the reaction solution was quenched by adding saturated sodium sulfite solution (30 mL). The reaction mixture was extracted by adding dichloromethane (100 mL×2 times). The organic phases were combined, washed with saturated brine (100 mL), dried over anhydrous sodium sulfate, filtered, concentrated through pressure concentration. The residue was dissolved in pyridine (5 mL), with 5-bromo-2-methoxypyridin-3-amine (882 mg, 4.345 mmol) added. The reaction mixture was kept at room temperature and reacted overnight. Upon completion of the reaction, the reaction was quenched by adding water (30 mL). The reaction mixture was extracted by adding dichloromethane (50 mL×2 times). The organic phases were combined, washed with saturated brine (50 mL), dried over anhydrous sodium sulfate, filtered, and concentrated under reduced pressure. The crude product obtained was purified by silica gel column chromatography (Eluent: methanol/dichloromethane=1/100 to 1/50). N-(5-bromo-2-methoxypyridin-3-yl)-3,5-difluoropyridine-4-sulfonamide was obtained, yield: 12.8%; ESI-MS(m/z): 382.0 [M+H]⁺.

### Step b-d): Preparation of (E)-3,5-difluoro-N-(2-methoxy-5-(4-(4-(4-oxopent-2-enoyl)piperazin-1-yl)quinazolin-6-yl)pyridin-3-yl)pyridine-4-sulfonamide

Referring to the preparation method of Example 83, Step b-d, (E)-3,5-difluoro-N-(2-methoxy-5-(4-(4-(4-oxopent-2-enoyl)piperazin-1-yl)quinazolin-6-yl)pyridin-3-yl)pyridine-4-sulfonamide was obtained, yield: 23.8%; ¹H NMR (400 MHz, DMSO-d₆) δ 11.19 (brs, 1H), 8.72 (s, 2H), 8.67 (s, 1H), 8.44 (s, 1H), 8.14-8.09 (m, 2H), 8.03-8.02 (m, 1H), 7.92 (d, J=16.0 Hz, 1H), 7.46 (d, J=16.0 Hz, 1H), 6.74 (d, J=16.0 Hz, 1H), 3.91-3.80 (m, 8H), 3.62 (s, 3H), 2.37 (s, 3H); ESI-MS(m/z): 610.0 [M+H]⁺.

### Example 103

### Preparation of (E)-3-fluoro-N-(2-methoxy-5-(4-(4-(4-oxopent-2-enoyl)piperazin-1-yl)quinazolin-6-yl)pyridin-3-yl)pyridine-4-sulfonamide

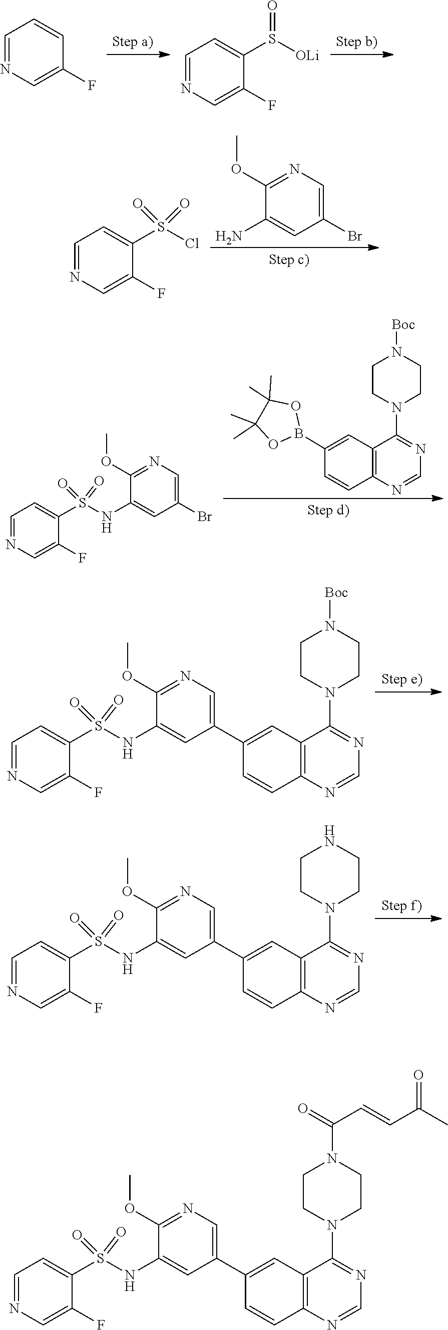

### Step a): Preparation of lithium 3-fluoropyridine-4-sulfinate

3-fluoropyridine (600 mg, 6.184 mmol) was placed in a three-necked bottle. In the presense of protective nitrogen, tetrahydrofuran (25 mL) was added. The reaction mixture was cooled to −78° C., with n-butyllithium (3.23 mL, 6.184 mmol, 1.6 M n-hexane solution) slowly added. After the addition was completed, the reaction mixture was kept at current temperature and reacted for 0.5 h, after that, with sulfur dioxide gas bubbled for 10 minutes, continued the reaction at room temperature for 2 h. Upon completion of the reaction, the reaction solution was directly spinned dry. Lithium 3-fluoropyridine-4-sulfinate was obtained. The product can be used directly in the next reaction without purification; ¹H NMR (400 MHz, DMSO-d₆) δ 8.46 (dd, J=4.8, 2.0 Hz, 1H), 8.41 (s, 1H), 7.60 (t, J=5.2 Hz, 1H); ESI-MS(m/z): 162.0 [M−Li+H+H]⁺.

### Step b): Preparation of 3-fluoropyridine-4-sulfonyl chloride

Lithium 3-fluoropyridine-4-sulfinate (2945 mg) was dispersed in dichloromethane (20 mL), with N-bromosuccinimide (1.7 g, 12.364 mmol) added in portions. After the addition was completed, in the presense of protective nitrogen, the reaction mixture was kept at room temperature and reacted for 2 h. Upon completion of the reaction, the reaction was quenched by adding water (30 mL), and the reaction mixture was extracted with dichloromethane (10 mL×2). The organic phases were combined, washed with water (40 mL), dried over anhydrous sodium sulfate, filtered. The filtrate was used directly in the next reaction.

### Step c): Preparation of N-(5-bromo-2-methoxypyridin-3-yl)-3-fluoropyridine-4-sulfonamide

5-bromo-2-methoxypyridin-3-amine (1.4 g, 6.800 mmol), 4-dimethylaminopyridine (76 mg, 0.620 mmol) and pyridine (2.4 g, 30.854 mmol) were added to the reaction flask which contained 3-fluoropyridine-4-sulfonyl chloride filtrate. In the presense of protective nitrogen, the reaction mixture was kept at room temperature and reacted for 15 h. Upon completion of the reaction, the reaction was quenched by adding saturated aqueous ammonium chloride solution (80 mL), and the reaction mixture was extracted with dichloromethane (50 mL×2). The organic phases were combined, dried over anhydrous sodium sulfate and filtered. The filtrate was concentrated under reduced pressure. The crude product obtained was purified by silica gel column chromatography (Eluent: dichloromethane/methanol=1/0 to 10/1). N-(5-bromo-2-methoxypyridin-3-yl)-3-fluoropyridine-4-sulfonamide was obtained, three-step yield: 16.8%; ¹H NMR (400 MHz, DMSO-d₆) δ 10.88 (s, 1H), 8.89 (d, J=2.0 Hz, 1H), 8.64 (dd, J=5.2, 0.8 Hz, 1H), 8.18 (d, J=2.0 Hz, 1H), 7.83 (d, J=2.4 Hz, 1H), 7.69 (t, J=6.0.4.8 Hz, 1H), 3.55 (s, 3H); ESI-MS(m/z): 361.9 [M+H]⁺.

### Step d-f): Preparation of (E)-3-fluoro-N-(2-methoxy-5-(4-(4-(4-oxopent-2-enoyl)piperazin-1-yl)quinazolin-6-yl)pyridin-3-yl)pyridine-4-sulfonamide

Referring to the preparation method of Example 83, Step b-d, (E)-3-fluoro-N-(2-methoxy-5-(4-(4-(4-oxopent-2-enoyl)piperazin-1-yl)quinazolin-6-yl)pyridin-3-yl)pyridine-4-sulfonamide was obtained, yield: 52.2%; ¹H NMR (400 MHz, DMSO-d₆) δ 10.81 (s, 1H), 8.89 (d, J=1.6 Hz, 1H), 8.67 (s, 1H), 8.63 (d, J=4.8 Hz, 1H), 8.53-8.49 (m, 1H), 8.17-8.10 (m, 2H), 8.06 (d, J=2.4 Hz, 1H), 7.92 (d, J=8.8 Hz, 1H), 7.72-7.67 (m, 1H), 7.47 (d, J=16.0 Hz, 1H), 6.74 (d, J=16.0 Hz, 1H), 3.96-3.78 (m, 8H), 3.61 (s, 3H), 2.38 (s, 3H); ESI-MS(m/z): 592.0 [M+H]⁺.

### Example 104

### Preparation of (E)-4-Chloro-N-(2-methoxy-5-(4-(4-(4-oxopent-2-enoyl)piperazin-1-yl)quinazoline-6-yl)pyridin-3-yl)-1-methyl-1H-pyrrole-2-sulfonamide

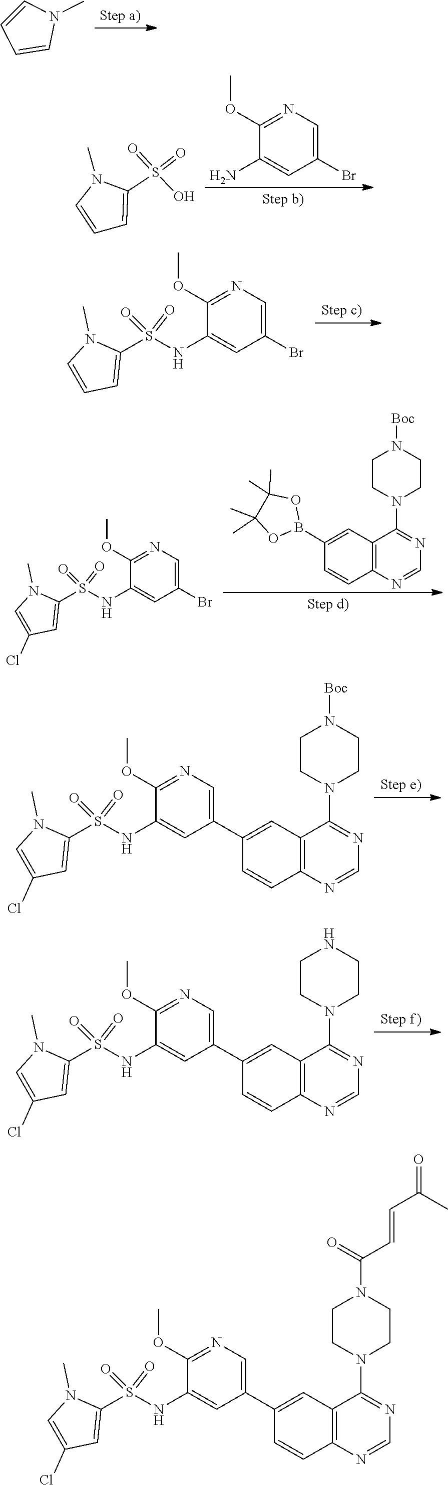

### Step a): Preparation of 1-methyl-1H-pyrrole-2-sulfonic acid

1-methyl-1H-pyrrole (300 mg, 3.704 mmol) was dissolved in dichloromethane (30 mL). After the reaction system was cooled to 0° C., chlorosulfonic acid (433 mg, 3.704 mmol) was diluted and slowly added dropwise to the reaction system. After the addition was completed, the reaction system was kept at 0° C. and stirred for 2 h. Upon completion of the reaction, the reaction solution can be used directly in the next reaction; ESI-MS (m/z): 160.1 [M−H]⁻.

### Step b): Preparation of N-(5-bromo-2-methoxypyridin-3-yl)-1-methyl-1H-pyrrole-2-sulfonamide

After the reaction solution in the previous step was cooled to 0° C., oxalyl chloride (1.9 g, 14.816 mmol) and N,N-dimethylformamide (8 mg, 0.111 mmol) were added to the system. After the addition was completed, the reaction mixture was heated to room temperature and reacted for 2 h. Upon completion of the reaction, the reaction solution was concentrated under reduced pressure. The residue was then dissolved in dichloromethane (20 mL), 4-dimethylaminopyridine (23 mg, 0.185 mmol), pyridine (878 mg, 11.112 mmol) and 5-bromo-2-methoxypyridin-3-amine (1.1 g, 5.556 mmol) were added to the system at room temperature. After the addition was completed, the reaction mixture was kept at room temperature and reacted for 3 h. Upon completion of the reaction, the reaction was quenched by adding water (50 mL). The reaction mixture was extracted by adding dichloromethane (100 mL×2 times). The organic phases were combined, washed with saturated brine (100 mL), dried over anhydrous sodium sulfate and filtered. The filtrate was concentrated under reduced pressure. The crude product obtained was purified by silica gel column chromatography (Eluent: petroleum ether/ethyl acetate=2/1 to 1/2). N-(5-bromo-2-methoxypyridin-3-yl)-1-methyl-1H-pyrrole-2-sulfonamide was obtained, yield: 19.5%; ESI-MS (m/z): 346.0 [M+H]⁺.

### Step c): Preparation of N-(5-bromo-2-methoxypyridin-3-yl)-4-chloro-1-methyl-1H-pyrrole-2-sulfonamide

N-(5-bromo-2-methoxypyridin-3-yl)-1-methyl-1H-pyrrole-2-sulfonamide (180 mg, 0.520 mmol) was dissolved in tetrahydrofuran/acetic acid mixed solvent (10 mL, v/v=1:1), with N-chlorosuccinimide (59 mg, 0.572 mmol) added. After the addition was completed, the reaction mixture was kept at 80° C. and stirred for 6 h. Upon completion of the reaction, the reaction was quenched by adding water (50 mL). The reaction mixture was extracted by adding dichloromethane (100 mL×2 times). The organic phases were combined, washed with saturated brine (100 mL), dried over anhydrous sodium sulfate and filtered. The filtrate was concentrated under reduced pressure. The crude product obtained was purified by silica gel column chromatography (Eluent: petroleum ether/ethyl acetate=2/1 to 1/2). N-(5-bromo-2-methoxypyridin-3-yl)-4-chloro-1-methyl-1H-pyrrole-2-sulfonamide was obtained, yield: 60.6%; ESI-MS(m/z): 380.8 [M+H]⁺.

### Step d-f): Preparation of (E)-4-chloro-N-(2-methoxy-5-(4-(4-(4-oxopent-2-enoyl)piperazin-1-yl)quinazoline-6-(yl)pyridin-3-yl)-1-methyl-1H-pyrrole-2-sulfonamide

Referring to the preparation method of Example 83, Step b-d, (E)-4-chloro-N-(2-methoxy-5-(4-(4-(4-oxopent-2-enoyl)piperazin-1-yl)quinazoline-6-(yl)pyridin-3-yl)-1-methyl-1H-pyrrole-2-sulfonamide was obtained, yield: 20.3%; ¹HNMR (400 MHz, DMSO-d₆) δ 9.43 (s, 1H), 8.67 (s, 1H), 8.39 (s, 1H), 8.11 (s, 1H), 8.07 (d, J=12.0 Hz, 1H), 7.97-7.92 (m, 2H), 7.48-7.44 (m, 2H), 6.74 (d, J=16.0 Hz, 1H), 6.40 (s, 1H), 3.92-3.89 (m, 6H), 3.86 (s, 3H), 3.84-3.80 (m, 2H), 3.56 (s, 3H), 2.37 (s, 3H); ESI-MS(m/z): 610.0 [M+H]⁺.

### Example 105

### Preparation of (E)-2,4-difluoro-N-(2-(methylamino)-5-(4-(4-(4-oxopent-2-enoyl)piperazin-1-yl)quinazolin-6-yl)pyridin-3-yl)benzenesulfonamide

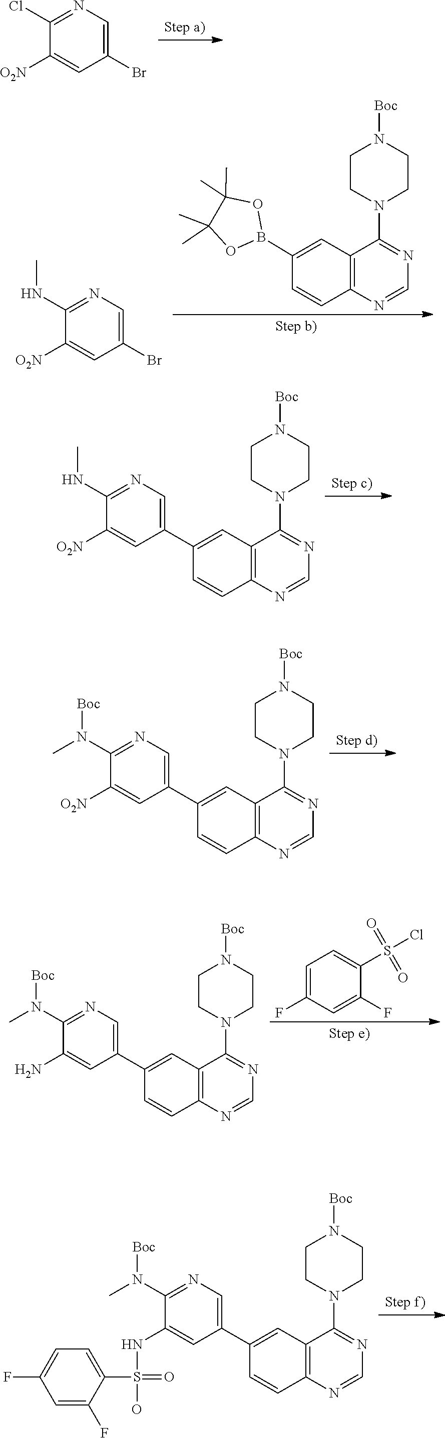

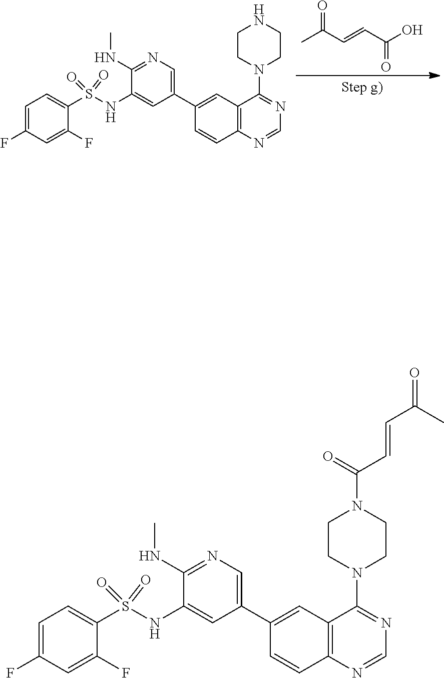

5-bromo-2-chloro-3-nitropyridine (5.0 g, 21.058 mmol), methylamine hydrochloride (4.3 g, 62.963 mmol), potassium carbonate (14.55 g, 105.290 mmol) were added to DMF (150 mL). After the addition was completed, the reaction mixture was stirred at room temperature and reacted overnight. Upon completion of the reaction, the reaction was quenched by adding water (100 mL). The reaction mixture was extracted with ethyl acetate (200) mL×2 times). The organic phases were combined, washed with saturated brine (100 mL), dried over anhydrous sodium sulfate and filtered. The filtrate was concentrated under reduced pressure. The crude product obtained was purified by silica gel column chromatography (Eluent: dichloromethane/methanol=100/1 to 50/1), 5-Bromo-N-methyl-3-nitropyridin-2-amine was obtained, yield: 96.2%; ESI-MS(m/z): 232.0 [M+H]⁺.

### Step b): Preparation of tert-butyl 4-(6-(6-(methylamino)-5-nitropyridin-3-yl)quinazolin-4-yl)piperazine-1-carboxylate

5-bromo-N-methyl-3-nitropyridin-2-amine (500 mg, 2.155 mmol), tert-butyl 4-(6-(4,4,5,5-tetramethyl-1,3,2-dioxaborolan-2-yl)quinazolin-4-yl)piperazine-1-carboxylate (949 mg, 2.155 mmol), Pd(dppf)Cl₂ (315 mg, 0.431 mmol) and cesium carbonate (2.1 g, 6.465 mmol) were added to dioxane/water mixed solvent (20 mL, v/v=10:1). After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (150 mL×2 times). The organic phases were combined, washed with saturated brine (100 mL), dried over anhydrous sodium sulfate and filtered. The filtrate was concentrated under reduced pressure. The crude product obtained was purified by silica gel column chromatography (Eluent: dichloromethane/methanol=100/1 to 40/1). Tert-butyl 4-(6-(6-(methylamino)-5-nitropyridin-3-yl)quinazolin-4-yl)piperazine-1-carboxylate was obtained, yield: 88.5%; ESI-MS(m/z): 466.2 [M+H]⁺.

### Step c): Preparation of tert-butyl 4-(6-(6-((tert-butoxycarbonyl)(methyl)amino)-5-nitropyridin-3-yl)quinazolin-4-yl)piperazine-1-carboxylate

Tert-butyl 4-(6-(6-(methylamino)-5-nitropyridin-3-yl)quinazolin-4-yl)piperazine-1-carboxylate (500 mg, 1.075 mmol), di-tert-butyl dicarbonate (704 mg, 3.225 mmol), 4-dimethylaminopyridine (6 mg, 0.054 mmol) and N,N-diisopropylethylamine (693 mg, 5.375 mmol) were added to DMF (10 mL). After the addition was completed, at 60° C. the reaction mixture was stirred and reacted for 2 h. Upon completion of the reaction, the reaction was quenched by adding water (50 mL). The reaction mixture was extracted with ethyl acetate (150 mL×2 times). The organic phases were combined, washed with saturated brine (100 mL), dried over anhydrous sodium sulfate and filtered. The filtrate was concentrated under reduced pressure. The crude product obtained was purified by silica gel column chromatography (Eluent: dichloromethane/methanol=100/1 to 50/1). Tert-butyl 4-(6-(6-((tert-butoxycarbonyl)(methyl)amino)-5-nitropyridin-3-yl)quinazolin-4-yl)piperazine-1-carboxylate was obtained, yield: 90.5%; ESI-MS(m/z): 566.3 [M+H]⁺.

### Step d): Preparation of tert-butyl 4-(6-(5-amino-6-((tert-butoxycarbonyl)(methyl)amino)pyridin-3-yl)quinazolin-4-yl)piperazine-1-carboxylate

Tert-butyl 4-(6-(6-((tert-butoxycarbonyl)methyl)amino)-5-nitropyridin-3-yl)quinazolin-4-yl)piperazine-1-carboxylate (500 mg, 0.884 mmol), Fe powder (494 mg, 8.840 mmol), ammonium chloride (47 mg, 0.884 mmol) were added to ethanol/water=5/1 mixed solvent (30 mL). After the addition was completed, at 90° C., the reaction mixture was stirred and reacted for 4 h. Upon completion of the reaction. The reaction was quenched by adding water (50 mL). The reaction mixture was extracted with ethyl acetate (200 mL×2 times). The organic phases were combined, washed with saturated brine (100 mL), dried over anhydrous sodium sulfate and filtered. The filtrate was concentrated under reduced pressure. Tert-butyl 4-(6-(5-amino-6-((tert-butoxycarbonyl)(methyl)amino)pyridin-3-yl)quinazolin-4-yl)piperazine-1-carboxylate was obtained, yield: 68.6%; ESI-MS(m/z): 536.3 [M+H]⁺.

### Step e): tert-butyl 4-(6-(6-((tert-butoxycarbonyl)(methyl)amino)-5-((2,4-difluorophenyl)sulfonamido)pyridin-3-yl)quinazolin-4-yl)piperazine-1-carboxylate

Tert-butyl 4-(6-(5-amino-6-((tert-butoxycarbonyl)(methyl)amino)pyridin-3-yl)quinazolin-4-yl)piperazine-1-carboxylate (300 mg, 0.560 mmol), 2,4-difluorobenzenesulfonyl chloride (357 mg, 1.680 mmol) were added to pyridine (5 mL). After the addition was completed, at 80° C., the reaction mixture was stirred and reacted overnight. Upon completion of the reaction, the reaction solution was diluted by adding water (50 mL) and extracted with ethyl acetate (200 mL 2 times). The organic phases were combined, washed with saturated brine (100 mL), dried over anhydrous sodium sulfate and filtered. The filtrate was concentrated under reduced pressure until completely dried. The crude product obtained was purified by silica gel column chromatography (Eluent: dichloromethane/methanol=100/1 to 50/1). Tert-butyl 4-(6-(6-((tert-butoxycarbonyl)(methyl)amino)-5-((2,4-difluorophenyl)sulfonamido)pyridin-3-yl)quinazolin-4-yl)piperazine-1-carboxylate was obtained, yield: 42.6%; ESI-MS(m/z): 712.3 [M+H]⁺.

### Step f): Preparation of 2,4-difluoro-N-(2-(methylamino)-5-(4-(piperazin-1-yl)quinazolin-6-yl)pyridin-3-yl)benzenesulfonamide trifluoroacetate

Tert-butyl 4-(6-(6-((tert-butoxycarbonyl)(methyl)amino)-5-((2,4-difluorophenyl)sulfonamido)pyridin-3-yl)quinazolin-4-yl)piperazine-1-carboxylate (170 mg, 0.239 mmol) was dissolved in dichloromethane (3 mL). Under the ice bath condition, TFA (1 mL) was slowly added dropwise. After the addition was completed, the reaction mixture was stirred at room temperature for 1 h. Upon completion of the reaction, the reaction mixture was concentrated under reduced pressure. The crude product was slurried with methyl tert-butyl ether (5 mL) to precipitate solid and filtered. Solid phase 2,4-difluoro-N-(2-(methylamino)-5-(4-(piperazin-1-yl)quinazolin-6-yl)pyridin-3-yl)benzenesulfonamide trifluoroacetate was obtained, yield: 90.2%; ESI-MS(m/z): 512.2 [M+H]⁺.

### Step g): Preparation of (E)-2,4-difluoro-N-(2-(methylamino)-5-(4-(4-(4-oxopent-2-enoyl)piperazin-1-yl)quinazolin-6-yl)pyridin-3-yl)benzenesulfonamide

2,4-difluoro-N-(2-(methylamino)-5-(4-(piperazin-1-yl)quinazolin-6-yl)pyridin-3-yl)benzenesulfonamide trifluoroacetate (110 mg, 0.176 mmol) was dissolved in tetrahydrofuran (2 mL). The reaction system was cooled to −78° C., with N,N-diisopropylethylamine (86 mg, 1.056 mmol), (E)-4-oxopent-2-enoic acid (22 mg, 0.194 mmol) and 50% T₃P ethyl acetate solution (224 mg, 0.352 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC (Method 2). (E)-2,4-Difluoro-N-(2-(methylamino)-5-(4-(4-(4-oxopent-2-enoyl)piperazin-1-yl)quinazolin-6-yl)pyridin-3-yl)benzenesulfonamide was obtained, yield: 16.7%; ¹H NMR (400 MHz, DMSO-d₆) δ 8.64 (s, 1H), 8.23 (s, 1H), 7.94 (s, 1H), 7.89-7.79 (m, 3H), 7.54-7.43 (m, 3H), 7.23-7.19 (m, 1H), 6.74 (d, J=16.0 Hz, 1H), 3.89-3.78 (m, 8H), 2.86 (s, 3H), 2.37 (s, 3H); ESI-MS(m/z): 608.0 [M+H]⁺.

Referring to the preparation method of Example 105 and using the corresponding raw materials, the compounds in the following examples were prepared.

| Num- | | | |
|---|---|---|---|
| ber | Name | Structure | ¹H NMR and MS |
| Ex- ample 106 | N-(5-(4-(4- acryloylpiperazin- 1-yl)quinazolin- 6-yl)-2- (cyclopentyloxy) pyridin-3-yl)-2,4- difluoro- benzene- sulfonamide | 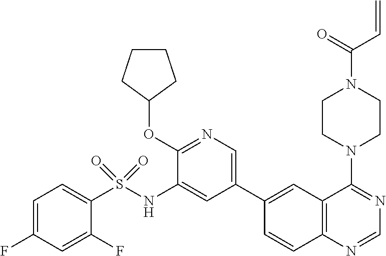 [SMILES: C=CC(=O)N1CCN(c2ncnc3ccc(-c4cnc(OC5CCCC5)c(NS(=O)(=O)c5ccc(F)cc5F)c4)cc23)CC1] | ¹H NMR (400 MHz, DMSO-d₆) δ 8.63 (s, 1H), 8.06-7.97 (m, 3H), 7.89- 7.77 (m, 3H), 7.34-7.29 (m, 1H), 7.11- 7.07 (m, 1H), 6.86-6.79 (m, 1H), 6.17 (d, J = 16.0 Hz, 1H), 5.73 (d, J = 16.0 Hz, 1H), 5.33-5.28 (m, 1H), 3.83-3.78 (m, 9H), 1.94-1.87 (m, 2H), 1.70-1.53 (m, 6H); ESI-MS(m/z): 621.7 [M + H]⁺. |
| Ex- ample 107 | (E)-N-(2- (cyclopentyloxy)-5- (4-(4-(4-oxopent-2- enoyl)piperazin-1- yl)quinazolin-6- yl)pyridin-3-yl)- 2,4-difluoro- benzene- sulfonamide | 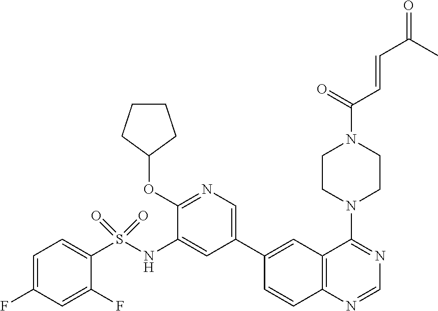 [SMILES: CC(=O)/C=C/C(=O)N1CCN(c2ncnc3ccc(-c4cnc(OC5CCCC5)c(NS(=O)(=O)c5ccc(F)cc5F)c4)cc23)CC1] | ¹H NMR (400 MHz, DMSO-d₆) δ 8.66 (s, 1H), 8.46 (s, 1H), 8.14-8.09 (m, 2H), 8.04-7.91 (m, 1H), 7.92 (d, J = 8.0 Hz, 1H), 7.81-7.75 (m, 1H), 7.58-7.52 (m, 1H), 7.44 (d, J = 16.0 Hz, 1H), 7.23-7.19 (m, 1H), 6.74 (d, J = 16.0 Hz, 1H), 5.28-5.23 (m, 1H), 3.90-3.80 (m, 9H), 2.37 (s, 3H), 1.87- 1.78 (m, 2H), 1.64-1.51 (m, 2H), 1.49-1.45 (m, 4H); ESI-MS(m/z): 663.7 [M + H]⁺. |
| Ex- ample 108 | N-(2- (cyclopentyloxy)- 5-(4-(4-(2- fluoroacryloyl) piperazin-1- yl)quinazolin-6- yl)pyridin-3-yl)- 2,4-difluoro- benzene- sulfonamide | 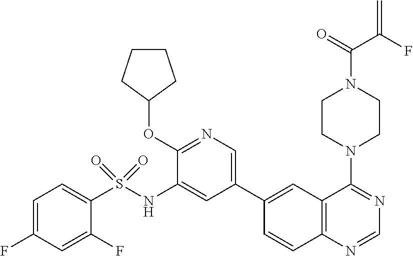 [SMILES: C=C(F)C(=O)N1CCN(c2ncnc3ccc(-c4cnc(OC5CCCC5)c(NS(=O)(=O)c5ccc(F)cc5F)c4)cc23)CC1] | ¹H NMR (400 MHz, DMSO-d₆) δ 10.15 (s, 1H), 8.65 (s, 1H), 8.44 (s, 1H), 8.12-8.09 (m, 2H), 8.02 (d, J = 4.0 Hz, 1H), 7.91 (d, J = 8.0 Hz, 1H), 7.79-7.74 (m, 1H), 7.56-7.50 (m, 1H), 7.22-7.17 (m, 1H), 5.36-7.31 (m, 1H), 5.25-5.19 (m, 1H), 3.91-3.79 (m, 9H), 1.86-1.78 (m, 2H), 1.61-1.51 (m, 2H), 1.49-1.44 (m, 4H); ESI-MS(m/z): 639.7 [M + H]⁺. |
| Ex- ample 109 | (E)-N-(2-ethoxy-5- (4-(4-(4-oxopent-2- enoyl)piperazin-1- yl)quinazolin-6- yl)pyridin-3-yl)- 2,4-difluoro- benzene- sulfonamide | 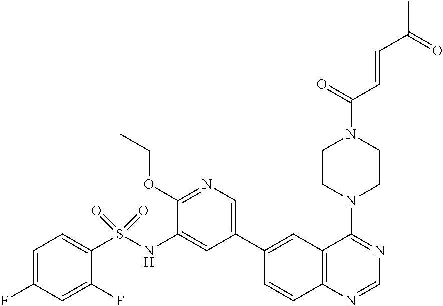 [SMILES: CCOc1ncc(-c2ccc3ncnc(N4CCN(C(=O)/C=C/C(C)=O)CC4)c3c2)cc1NS(=O)(=O)c1ccc(F)cc1F] | ¹H NMR (400 MHz, DMSO-d₆) δ 10.26 (s, 1H), 8.66 (s, 1H), 8.45 (d, J = 4.0 Hz, 1H), 8.14 (s, 1H), 8.10 (d, J = 8.0 Hz, 1H), 8.03 (d, J = 4.0 Hz, 1H), 7.92 (d, J = 8.0 Hz, 1H), 7.81- 7.75 (m, 1H), 7.59-7.54 (m, 1H), 7.45 (d, J = 16.0 Hz, 1H), 7.23-7.18 (m, 1H), 6.74 (d, J = 16.0 Hz, 1H), 4.19- 4.13 (m, 2H), 3.90-3.81 (m, 8H), 2.37 (s, 3H), 1.13 (t, J = 8.0 Hz, 3H); ESI- MS(m/z): 623.5 [M + H]⁺. |
| Ex- ample 110 | (E)-N-(2- cyclopropoxy-5-(4- (4-(4-oxopent-2- enoyl)piperazin-1- yl)quinazolin-6- yl)pyridin-3-yl)- 2,4-difluoro- benzene- sulfonamide | 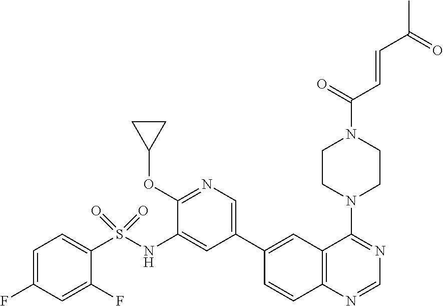 [SMILES: CC(=O)/C=C/C(=O)N1CCN(c2ncnc3ccc(-c4cnc(OC5CC5)c(NS(=O)(=O)c5ccc(F)cc5F)c4)cc23)CC1] | ¹H NMR (400 MHz, DMSO-d₆) δ 10.25 (s, 1H), 8.67 (s, 1H), 8.51 (d, J = 4.0 Hz, 1H), 8.15 (s, 1H), 8.11 (d, J = 4.0 Hz, 1H), 8.03 (d, J = 4.0 Hz, 1H), 7.92 (d, J = 8.0 Hz, 1H), 7.76- 7.70 (m, 1H), 7.61-7.56 (m, 1H), 7.45 (d, J = 16.0 Hz, 1H), 7.23-7.18 (m, 1H), 6.74 (d, J = 16.0 Hz, 1H), 4.17- 4.13 (m, 1H), 3.90-3.81 (m, 8H), 2.37 (s, 3H), 0.69-0.64 (m, 2H), 0.35-0.31 (m, 2H); ESI-MS(m/z): 635.5 [M + H]⁺. |

### Example 111

### Preparation of (E)-2-fluoro-N-(2-methoxy-5-(4-(4-(4-oxopent-2-enoyl)piperazin-1-yl)quinazoline-6-(yl)pyridin-3-yl)-4-methylbenzenesulfonamide

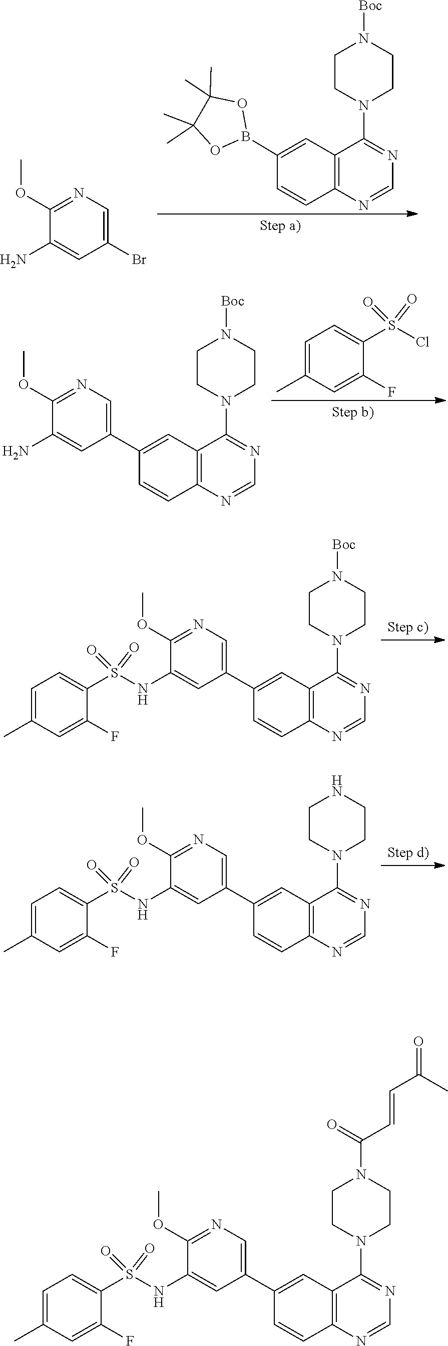

### Step a): Preparation of tert-butyl 4-(6-(5-amino-6-methoxypyridin-3-yl)quinazolin-4-yl)piperazine-1-carboxylate

5-bromo-2-methoxypyridin-3-amine (1.0 g, 4.932 mmol), tert-butyl 4-(6-(4,4,5,5-tetramethyl-1,3,2-dioxaborolan-2-yl)quinazolin-4-yl)piperazine-1-carboxylate (2.2 g, 4.932 mmol), Pd(dppf)Cl₂ (361 mg, 0.493 mmol) and cesium carbonate (4.8 g, 14.795 mmol) were added to dioxane (45 mL) and water (15 mL) mixed solvent. After the addition was completed, in the presense of protective nitrogen, the reaction mixture was heated to 100° C. and stirred for 13 h. Upon completion of the reaction, the reaction mixture was cooled to room temperature and the reaction was quenched by adding saturated ammonium chloride (80 mL). The reaction mixture was extracted with ethyl acetate (80 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=5/1 to 1/2). Tert-butyl 4-(6-(5-amino-6-methoxypyridin-3-yl)quinazolin-4-yl)piperazine-1-carboxylate was obtained, yield: 77.4%, ESI-MS(m/z): 437.1 [M+H]4.

### Step b): Preparation of tert-butyl 4-(6-(5-((3-fluoro-5-methyl-2-sulfamoylbenzyl)oxy)pyridin-3-yl)quinazolin-4-yl)piperazine-1-carboxylate

Tert-butyl 4-(6-(5-amino-6-methoxypyridin-3-yl)quinazolin-4-yl)piperazine-1-carboxylate (150 mg, 0.340 mmol), 2-fluoro-4-methylbenzenesulfonyl chloride (78 mg, 0.372 mmol), 4-dimethylaminopyridine (4 mg, 0.034 mmol) and pyridine (81 mg, 1.023 mmol) were dissolved in dichloromethane (10 mL). After the addition was completed, the reaction mixture was stirred at room temperature for 18 h. Upon completion of the reaction, the reaction was quenched by adding saturated aqueous ammonium chloride solution (30 mL) and the reaction mixture was extracted with dichloromethane (30 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=10/1 to 0/1). Tert-butyl 4-(6-(5-((3-fluoro-5-methyl-2-sulfamoylbenzyl)oxy)pyridin-3-yl)quinazolin-4-yl)piperazine-1-carboxylate was obtained, yield: 38.3%; ESI-MS(m/z): 609.2 [M+H]⁺.

### Step c): Preparation of 2-fluoro-N-(2-methoxy-5-(4-(piperazin-1-yl)quinazolin-6-yl)pyridin-3-yl)-4-methylbenzenesulfonamide trifluoroacetate

Tert-butyl 4-(6-(5-((3-fluoro-5-methyl-2-sulfamoylbenzoyl)oxy)pyridin-3-yl)quinazolin-4-yl)piperazine-1-carboxylate (90 mg, 0.132 mmol) was dissolved in dichloromethane (6 mL). Under the ice bath condition, TFA (2 mL) was slowly added dropwise. After the addition was completed, the reaction mixture was stirred at room temperature for 1 h. Upon completion of the reaction, the reaction mixture was concentrated under reduced pressure. The product obtained was dissolved in 4 mL methyl tert-butyl ether. The reaction mixture was stirred at room temperature for 0.5 h, after solid precipitation appeared, filtered to obtain 2-fluoro-N-(2-methoxy-5-(4-(piperazin-1-yl)quinazolin-6-yl)pyridin-3-yl)-4-methylbenzenesulfonamide trifluoroacetate, yield: 97.3%; ESI-MS(m/z): 509.3 [M+H]⁺.

### Step d): Preparation of (E)-2-fluoro-N-(2-methoxy-5-(4-(4-(4-oxopent-2-enoyl)piperazin-1-yl)quinazoline-6-(yl)pyridin-3-yl)-4-methylbenzenesulfonamide

2-fluoro-N-(2-methoxy-5-(4-(piperazin-1-yl)quinazolin-6-yl)pyridin-3-yl)-4-methylbenzenesulfonamide trifluoroacetate (122 mg, 0.132 mmol), (E)-4-oxopent-2-enoic acid (16 mg, 0.143 mmol), N,N-diisopropylethylamine (84 mg, 0.654 mmol) were dissolved in tetrahydrofuran (2 mL), at −78° C., with 1-propylphosphonic anhydride (91 mg, 0.143 mmol, 50% wt %) slowly added. Upon completion of the reaction, with 1 mL acetonitrile added, the reaction solution was filtered and purified by Prep-HPLC (Method 2). (E)-2-fluoro-N-(2-methoxy-5-(4-(4-(4-oxopent-2-enoyl)piperazin-1-yl)quinazoline-6-(yl)pyridin-3-yl)-4-methylbenzenesulfonamide was obtained, yield: 45.8%; ¹H NMR (400 MHz, DMSO-d₆) δ 10.17 (s, 1H), 8.67 (s, 1H), 8.46-8.40 (m, 1H), 8.13-8.03 (m, 2H), 7.98 (d, J=2.4 Hz, 1H), 7.92 (d, J=8.8 Hz, 1H), 7.59 (t, J=7.9 Hz, 1H), 7.46 (d, J=15.6 Hz, 1H), 7.29 (d, J=11.2 Hz, 1H), 7.16-7.09 (m, 1H), 6.74 (d, J=15.6 Hz, 1H), 3.93-3.78 (m, 8H), 3.69 (s, 3H), 2.37-2.35 (m, 6H); ESI-MS(m/z): 605.0 [M+H]⁺.

### Example 112

### Preparation of (S,E)-N-(5-(4-(3-(cyanomethyl)-4-(4-oxopent-2-enoyl)piperazin-1-yl)quinazolin-6-yl)-2-methoxypyridin-3-yl)-2,4-difluorobenzenesulfonamide

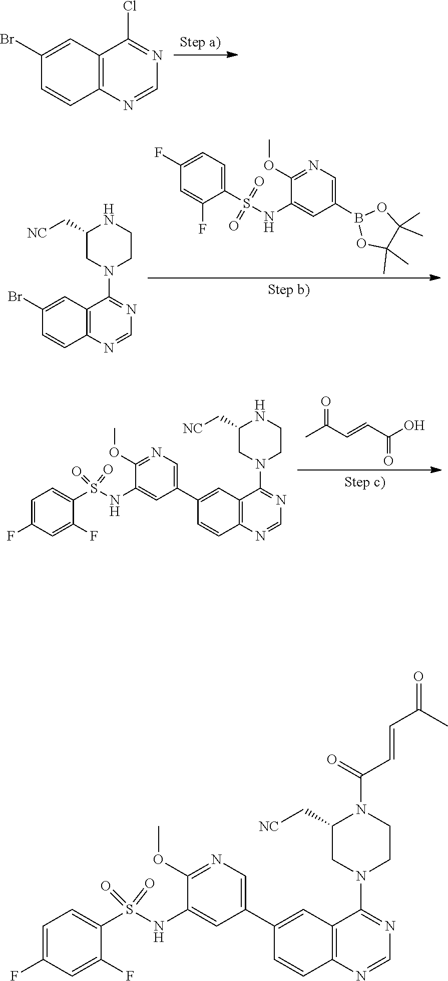

Step a): Preparation of(S)-2-(4-(6-bromoquinazolin-4-yl) piperazin-2-yl)acetonitrile

6-bromo-4-chloroquinazoline (600 mg, 2.464 mmol) and (S)-2-(piperazin-2-yl)acetonitrile dihydrochloride (537 mg, 2.711 mmol) were dissolved in DMF (8 mL), with N,N-diisopropylethylamine (955 mg, 7.389 mmol) added. After the addition was completed, the reaction mixture was kept at 50° C. and stirred for 1 h. Upon completion of the reaction, the reaction was quenched by adding water (50 mL). The reaction mixture was extracted with ethyl acetate (100 mL 2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=1/1 to 1/5). (S)-2-(4-(6-bromoquinazolin-4-yl)piperazin-2-yl)acetonitrile was obtained, yield: 95.2%; ESI-MS(m/z): 332.0 [M+H]⁺.

### Step b): Preparation of (S)—N-(5-(4-(3-(cyanomethyl)piperazin-1-yl)quinazolin-6-yl)-2-methoxypyridin-3-yl)-2,4-difluorobenzenesulfonamide

(S)-2-(4-(6-bromoquinazolin-4-yl)piperazin-2-yl)acetonitrile (780 mg, 2.350 mmol), 2,4-difluoro-N-(2-hydroxy-5-(4,4,5,5-tetramethyl-1,3,2-dioxaborolan-2-yl)pyridin-3-yl)benzenesulfonamide (1.1 g, 2.581 mmol), Pd(dppf)Cl₂ (172 mg, 0.235 mmol) and cesium carbonate (2.3 g, 7.059 mmol) were dissolved in dioxane/water mixed solvent (8 mL, v/v=5:1). After the addition was completed, in the presense of protective nitrogen, the reaction mixture was heated to 100° C. and stirred for 1 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated under reduced pressure. The crude product obtained was purified by silica gel column chromatography (Eluent: dichloromethane/methanol=50/1 to 20/1). (S)—N-(5-(4-(3-(cyanomethyl)piperazin-1-yl)quinazolin-6-yl)-2-methoxypyridin-3-yl)-2,4-difluorobenzenesulfonamide was obtained, yield: 77.2%; ESI-MS(m/z): 552.2 [M+H]⁺.

### Step c): Preparation of (S,E)-N-(5-(4-(3-(cyanomethyl)-4-(4-oxopent-2-enoyl)piperazin-1-yl)quinazolin-6-yl)-2-methoxypyridin-3-yl)-2,4-difluorobenzenesulfonamide

(S)—N-(5-(4-(3-(cyanomethyl)piperazin-1-yl)quinazolin-6-yl)-2-methoxypyridin-3-yl)-2,4-difluorobenzenesulfonamide (120 mg, 0.217 mmol) and 3-acetylacrylic acid (50 mg, 0.438 mmol) were dissolved in THF (2 mL). The reaction system was cooled to −78° C. with N,N-diisopropylethylamine (85 mg, 0.658 mmol) and 1-propylphosphonic anhydride (415 mg, 0.653 mmol, 50% wt) added successively. After the addition was completed, the reaction mixture was kept at −78° C. and stirred for 1 h. Upon completion of the reaction, the reaction was quenched by adding water (10 mL), and the reaction mixture was extracted with dichloromethane (30 mL×2). The organic phases were combined, dried over anhydrous sodium sulfate and filtered. The filtrate was concentrated under reduced pressure. The crude product obtained was purified by Prep-HPLC (Method 2). (S,E)-N-(5-(4-(3-(cyanomethyl)-4-(4-oxopent-2-enoyl)piperazin-1-yl)quinazolin-6-yl)-2-methoxypyridin-3-yl)-2,4-difluorobenzenesulfonamide was obtained, yield: 33.0%; ¹H NMR (400 MHz, DMSO-d₆) δ 10.30 (s, 1H), 8.70-8.66 (m, 1H), 8.49 (d, J=8.0 Hz, 1H), 8.21 (s, 1H), 8.13-8.07 (m, 1H), 8.05-8.00 (m, 1H), 7.93 (d, J=8.0 Hz, 1H), 7.81-7.73 (m, 1H), 7.59-7.40 (m, 2H), 7.24-7.17 (m, 1H), 6.82-6.69 (m, 1H), 5.07-4.89 (m, 1H), 4.49-4.25 (m, 2H), 4.18-3.85 (m, 1H), 3.68 (s, 3H), 3.60-3.37 (m, 3H), 3.25-3.00 (m, 2H), 2.40-2.35 (m, 3H); ESI-MS(m/z): 648.0 [M+H]⁺.

Referring to the preparation method of Example 112 and using the corresponding raw materials, the compounds in the following examples were prepared.

| Number | Name | Structure | ¹H NMR and MS |
|---|---|---|---|
| Example 113 | (S)-N-(5-(4-(3- (cyanomethyl)-4-(2- fluoroacryloyl) piperazin-1- yl)quinazolin-6-yl)- 2-methoxypyridin- 3-yl)-2,4- difluoro- benzenesulfonamide | 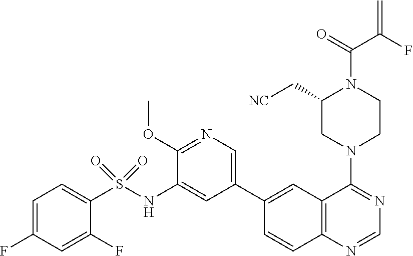 [SMILES: C=C(F)C(=O)N1CCN(c2ncnc3ccc(-c4cnc(OC)c(NS(=O)(=O)c5ccc(F)cc5F)c4)cc23)C[C@@H]1CC#N] | ¹H NMR (400 MHz, DMSO-d₆) δ 10.33 (s, 1H), 8.69 (s, 1H), 8.52-8.50 (m, 1H), 8.21-8.18 (m, 1H), 8.12-8.08 (m, 1H), 8.05-8.03 (m, 1H), 7.94 (d, J = 8.0 Hz, 1H), 7.81-7.73 (m, 1H), 7.60-7.53 (m, 1H), 7.24-7.18 (m, 1H), 5.44-5.37 (m, 1H), 5.36-5.21 (m, 1H), 4.92 (s, 1H), 4.37-4.27 (m, 2H), 4.17- 3.78 (m, 2H), 3.68 (s, 3H), 3.50-3.44 (m, 1H), 3.41-3.31 (m, 2H), 3.10-3.03 (m, 1H); ESI-MS(m/z): 624.0 [M + H]+. |
| Example 114 | (S)-N-(5-(4-(4- acryloyl-3- (cyanomethyl) piperazin-1- yl)quinazolin-6-yl)- 2-methoxypyridin- 3-yl)-2,4- difluoro- benzenesulfonamide | 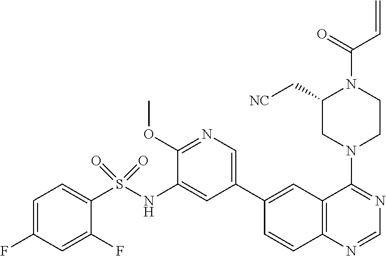 [SMILES: C=CC(=O)N1CCN(c2ncnc3ccc(-c4cnc(OC)c(NS(=O)(=O)c5ccc(F)cc5F)c4)cc23)C[C@@H]1CC#N] | ¹H NMR (400 MHz, DMSO-d₆) δ 10.31 (s, 1H), 8.67 (s, 1H), 8.42 (m, 1H), 8.20-8.17 (m, 1H), 8.07 (d, J = 8.0 Hz, 1H), 8.01-7.97 (m, 1H), 7.92 (d, J = 8.0 Hz, 1H), 7.81-7.74 (m, 1H), 7.55-7.49 (m, 1H), 7.22-7.16 (m, 1H), 6.95-6.78 (m, 1H), 6.22-6.16 (m, 1H), 5.78 (d, J = 8.0 Hz, 1H), 5.07- 4.82 (m, 1H), 4.49-4.20 (m, 3H), 4.15-3.79 (m, 1H), 3.69 (s, 3H), 3.60- 3.41 (m, 2H), 3.19-2.98 (m, 2H); ESI- MS(m/z): 606.0 [M + H]⁺. |
| Example 115 | (S)-N-(5-(4-(3- (cyanomethyl)-4-(2- fluoroacryloyl) piperazin-1- yl)quinazolin-6-yl)- 2-methoxypyridin- 3-yl)-2,6- difluoro- benzenesulfonamide | 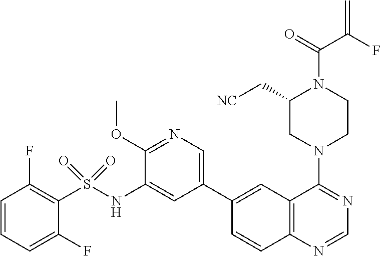 [SMILES: C=C(F)C(=O)N1CCN(c2ncnc3ccc(-c4cnc(OC)c(NS(=O)(=O)c5c(F)cccc5F)c4)cc23)C[C@@H]1CC#N] | ¹H NMR (400 MHz, DMSO-d₆) δ 10.59 (s, 1H), 8.69 (s, 1H), 8.54 (d, J = 2.4 Hz, 1H), 8.21 (d, J = 2.0 Hz, 1H), 8.14-8.04 (m, 2H), 7.95 (d, J = 8.8 Hz, 1H), 7.76-7.68 (m, 1H), 7.32- 7.24 (m, 2H), 5.48-5.20 (m, 2H), 4.94-4.92 (m, 1H), 4.40-4.24 (m, 2H), 4.09-3.96 (m, 2H), 3.65 (s, 3H), 3.54- 3.35 (m, 3H), 3.08-3.03 (m, 1H); ESI- MS(m/z): 624.0 [M + H]+. |
| Example 116 | (S,E)-N-(5-(4-(3- (cyanomethyl)-4-(4- oxopent-2- enoyl)piperazin-1- yl)quinazolin-6-yl)- 2-methoxypyridin- 3-yl)-2,6- difluoro- benzenesulfonamide | 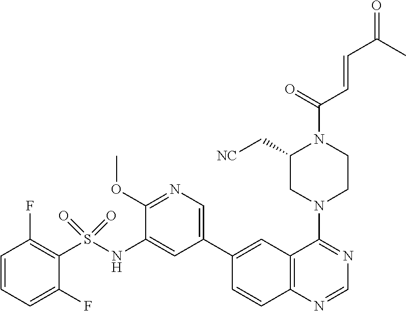 [SMILES: COc1ncc(-c2ccc3ncnc(N4CCN(C(=O)/C=C/C(C)=O)[C@@H](CC#N)C4)c3c2)cc1NS(=O)(=O)c1c(F)cccc1F] | ¹H NMR (400 MHz, DMSO-d₆) δ 10.58 (s, 1H), 8.69 (d, J = 4.8 Hz, 1H), 8.52 (d, J = 10.4 Hz, 1H), 8.22 (s, 1H), 8.15-8.01 (m, 2H), 7.95-7.93 (m, 1H), 7.75-7.68 (m, 1H), 7.56-7.42 (m, 1H), 7.27 (t, J = 9.6 Hz, 2H), 6.81-6.70 (m, 1H), 5.03-4.91 (m, 1H), 4.49-4.24 (m, 3H), 4.20-3.82 (m, 1H), 3.65 (d, J = 4.4 Hz, 3H), 3.60-3.35 (m, 2H), 3.24-3.17 (m, 1H), 3.08-3.01 (m, 1H), 2.38 (d, J = 12.4 Hz, 3H); ESI-MS(m/z): 648.0 [M + H]⁺. |
| Example 117 | (S)-N-(5-(4-(4- acryloyl-3- (cyanomethyl) piperazin-1- yl)quinazolin-6-yl)- 2-methoxypyridin- 3-yl)-2,6- difluoro- benzenesulfonamide | 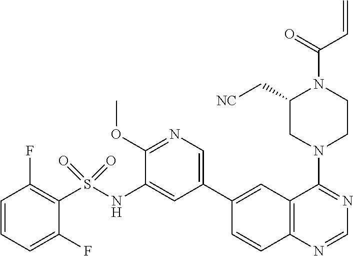 [SMILES: C=CC(=O)N1CCN(c2ncnc3ccc(-c4cnc(OC)c(NS(=O)(=O)c5c(F)cccc5F)c4)cc23)C[C@@H]1CC#N] | ¹H NMR (400 MHz, DMSO-d₆) δ 10.58 (s, 1H), 8.68 (s, 1H), 8.46-8.39 (m, 1H), 8.26-8.14 (m, 1H), 8.12-7.98 (m, 2H), 7.93 (d, J = 8.8 Hz, 1H), 7.72-7.68 (m, 1H), 7.25 (t, J = 9.2 Hz, 2H), 6.95-6.82 (m, 1H), 6.26-6.15 (m, 1H), 5.78 (d, J = 10.4 Hz, 1H), 5.05- 4.76 (m, 1H), 4.53-4.18 (m, 3H), 4.10-3.78 (m, 1H), 3.66 (s, 3H), 3.61- 3.38 (m, 2H), 3.21-2.94 (m, 2H); ESI- MS(m/z): 606.0 [M + H]⁺. |

### Example 118

### Preparation of (E)-5-fluoro-N-(2-methoxy-5-(4-(4-(4-oxopent-2-enoyl)piperazin-1-yl)quinazolin-6-yl)pyridin-3-yl)pyridine-2-sulfonamide

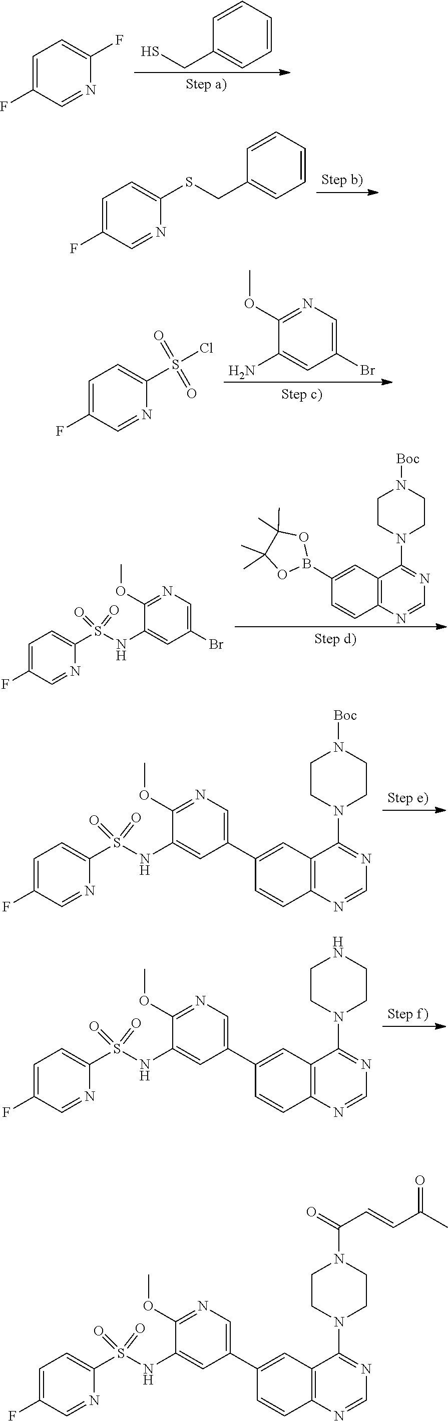

### Step a): Preparation of 2-(benzylthio)-5-fluoropyridine

2,5-difluoropyridine (1.15 g, 10.000 mmol), phenylmethanethiol (1.118 g, 9.000 mmol), Cs₂CO (3.26 g, 10.000 mmol) and DMSO (30 mL) were added to the reaction flask. The reaction mixture was stirred and reacted for 2 h at room temperature. Upon completion of the reaction, the reaction was quenched by adding water (150 mL). The reaction mixture was extracted with ethyl acetate (100 mL×3). The organic phases were combined, washed with saturated brine (100 mL), dried over anhydrous sodium sulfate, completely concentrated under reduced pressure. The residue was purified by silica gel column chromatography (Eluent: petroleum ether/ethyl acetate=100/1), 2-(Benzylthio)-5-fluoropyridine was obtained, yield: 55.5%; ESI-MS (m/z): 220.2 [M+H]⁺.

### Step b): Preparation of 5-fluoropyridine-2-sulfonyl chloride

2-(Benzylthio)-5-fluoropyridine (1.217 g, 5.550 mmol) and acetonitrile (20 mL) were added to the reaction flask, stirred to dissolve, with glacial acetic acid (1.2 mL) and water (700 uL) added. At 0° C., under the condition of stirring, 1,3-dichloro-5,5-dimethylimidazolidine-2,4-dione (2.187 g, 11.100 mmol) was slowly added to the system. After the addition was completed, the reaction mixture was stirred and reacted for 2 h at 0° C. After completion of the reaction, the reaction was quenched with saturated brine (100 mL). The reaction mixture was extracted with ethyl acetate (100 mL×3). The organic phases were combined, dried over anhydrous sodium sulfate, completely concentrated under reduced pressure. 5-fluoropyridine-2-sulfonyl chloride was obtained. The product can be used directly in the next reaction without further purification.

### Step c): Preparation of N-(5-bromo-2-methoxypyridin-3-yl)-5-fluoropyridine-2-sulfonamide

5-fluoropyridine-2-sulfonyl chloride (1085 mg, 5.550 mmol), 5-bromo-2-methoxypyridin-3-amine (1127 mg, 5.550 mmol), pyridine (1305 mg, 16.65 mmol) and DMAP (870 mg, 6.720 mmol) were added to dichloromethane (20 mL). The reaction solution was stirred at room temperature for 16 h. Upon completion of the reaction, the reaction was quenched by adding water (50 mL). The reaction mixture was extracted with ethyl acetate (50 mL×3). The organic phases were combined, washed with saturated brine (50 mL), dried over anhydrous sodium sulfate and filtered. The filtrate was concentrated under reduced pressure. The residue was purified by silica gel column chromatography (Eluent: petroleum ether/ethyl acetate=5/1). N-(5-bromo-2-methoxypyridin-3-yl)-5-fluoropyridine-2-sulfonamide was obtained, yield: 59.0%; ESI-MS (m/z): 362.2 [M+H]⁺.

### Step d): Preparation of tert-butyl 4-(6-(5-((5-fluoropyridine)-2-sulfonamido)-6-methoxypyridin-3-yl)quinazolin-4-yl)piperazine-1-carboxylate

Tert-butyl 4-(6-(4,4,5,5-tetramethyl-1,3,2-dioxaborolan-2-yl)quinazolin-4-yl)piperazine-1-carboxylate (594 mg, 1.350 mmol). N-(5-bromo-2-methoxypyridin-3-yl)-5-fluoropyridine-2-sulfonamide (489 mg, 1.350 mmol). Cs₂CO₃ (880 mg, 2.700 mmol), Pd(dppf)Cl₂ (98 mg, 0.135 mmol), water (1 mL) and 1,4-dioxane (10 mL) were added to the reaction flask. With nitrogen purged three times, the reaction mixture was heated to 90° C., stirred and reacted for 2 h. Upon completion of the reaction, the reaction was quenched by adding water (100 ml). The reaction mixture was extracted with ethyl acetate (50 mL×3). The organic phases were combined, washed with saturated brine (50 mL), concentrated under reduced pressure. The residue was purified by silica gel column chromatography (Eluent: dichloromethane/methanol=50/1). Tert-butyl 4-(6-(5-((5-fluoropyridine)-2-sulfonamido)-6-methoxypyridin-3-yl)quinazolin-4-yl)piperazine-1-carboxylate was obtained, yield: 72.1%; ESI-MS (m/z): 596.5 [M+H]⁺.

### Step e): Preparation of 5-fluoro-N-(2-methoxy-5-(4-(piperazin-1-yl)quinazolin-6-yl)pyridin-3-yl)pyridine-2-sulfonamide trifluoroacetate

Tert-butyl 4-(6-(5-((5-fluoropyridine)-2-sulfonamido)-6-methoxypyridin-3-yl)quinazolin-4-yl)piperazine-1-carboxylate (476 mg, 0.800 mmol) and dichloromethane (5 mL) were added to the reaction flask, stirred to dissolve. At 0° C., under the condition of stirring, HCl/1,4-dioxane solution (4 M, 5 mL) was slowly added to the system. After the addition was completed, the reaction mixture was naturally heated to room temperature and reacted for 1 h. After completion of the reaction, the reaction mixture was concentrated under reduced pressure. The product can be used directly in the next reaction without further purification; ESI-MS (m/z): 496.2 [M+H]⁺.

### Step f): Preparation of (E)-5-fluoro-N-(2-methoxy-5-(4-(4-(4-oxopent-2-enoyl)piperazin-1-yl)quinazolin-6-yl)pyridin-3-yl)pyridine-2-sulfonamide

5-fluoro-N-(2-methoxy-5-(4-(piperazin-1-yl)quinazolin-6-yl)pyridin-3-yl)pyridine-2-sulfonamide trifluoroacetate (119 mg, 0.200 mmol), (E)-4-oxopent-2-enoic acid (23 mg, 0.200 mmol), N,N-diisopropylethylamine (129 mg, 1.000 mmol) and tetrahydrofuran (2 mL) were added to the reaction flask. In the dry ice/ethanol bath, at −78° C., T₃P (254 mg, 0.400 mmol, 50% ethyl acetate solution) was added to the reaction system under the condition of stirring. The reaction system was kept at −78° C. and reacted for 1 h. Upon completion of the reaction, the reaction was quenched by adding water (20 mL). The reaction mixture was extracted with ethyl acetate (20 mL×2). The organic phases were combined and washed with saturated sodium bicarbonate aqueous solution (20 mL), saturated brine (10 mL×2) successively, dried over anhydrous sodium sulfate and filtered. The filtrate was concentrated under reduced pressure. The residue was purified by silica gel column chromatography (Eluent: dichloromethane/methanol=20/1). The crude product was then purified by Prep-HPLC (Method 2). (E)-5-fluoro-N-(2-methoxy-5-(4-(4-(4-oxopent-2-enoyl)piperazin-1-yl)quinazolin-6-yl)pyridin-3-yl)pyridine-2-sulfonamide was obtained, yield: 68.7%; ¹H NMR (400 MHz, DMSO-d₆) δ 10.16 (s, 1H), 8.75 (d, J=4.0 Hz, 1H), 8.67 (s, 1H), 8.41 (s, 1H), 8.13-7.92 (m, 6H), 7.43 (d, J=16.0 Hz, 1H), 6.73 (d, J=16.0 Hz, 1H), 3.90-3.80 (m, 8H), 3.72 (s, 3H), 2.36 (s, 3H); ESI-MS(m/z): 592.6 [M+H]⁺.

Referring to the preparation method of Example 118 and using the corresponding raw materials, the compounds in the following examples were obtained.

| Num- | | | |
|---|---|---|---|
| ber | Name | Structure | ¹H NMR and MS |
| Ex- ample 119 | N-(5-(4-(4- acryloyl- piperazin-1- yl)quinazolin- 6-yl)-2- methoxypyridin- 3-yl)-5- fluoropyridine- 2-sulfonamide | 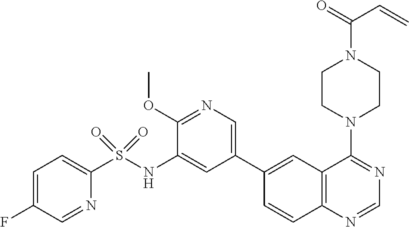 [SMILES: C=CC(=O)N1CCN(c2ncnc3ccc(-c4cnc(OC)c(NS(=O)(=O)c5ccc(F)cn5)c4)cc23)CC1] | ¹H NMR (400 MHz, DMSO-d₆) δ 10.21 (s, 1H), 8.75 (d, J = 4.0 Hz, 1H), 8.66 (s, 1H), 8.41 (s, 1H), 8.13- 7.91 (m, 6H), 6.85-6.78 (m, 1H), 6.16 (d, J = 16.0 Hz, 1H), 5.71 (d, J = 16.0 Hz, 1H), 3.89-3.78 (m, 8H), 3.72 (s, 3H); ESI-MS(m/z): 550.6 [M + H]⁺. |
| Ex- ample 120 | 5-fluoro-N-(5- (4-(4-(2- fluoroacryloyl) piperazin-1-yl) quinazolin-6-yl)- 2-methoxy- pyridin- 3-yl)pyridine-2- sulfonamide | 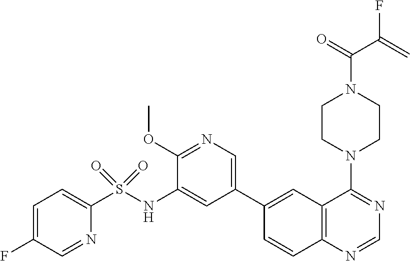 [SMILES: C=C(F)C(=O)N1CCN(c2ncnc3ccc(-c4cnc(OC)c(NS(=O)(=O)c5ccc(F)cn5)c4)cc23)CC1] | ¹H NMR (400 MHz, DMSO-d₆) δ 10.20 (s, 1H), 8.75 (d, J = 4.0 Hz, 1H), 8.67 (s, 1H), 8.42 (s, 1H), 8.13- 7.92 (m, 6H), 5.36-5.19 (m, 2H), 3.92-3.79 (m, 8H), 3.72 (s, 3H); ESI-MS(m/z): 568.6 [M + H]⁺. |
| Ex- ample 121 | (E)-5-fluoro- N-(3-fluoro-2- methoxy- 5-(4-(4- (4-oxopent- 2-enoyl) piperazin- 1-yl)quinazolin- 6-yl)phenyl) pyridine- 2-sulfonamide | 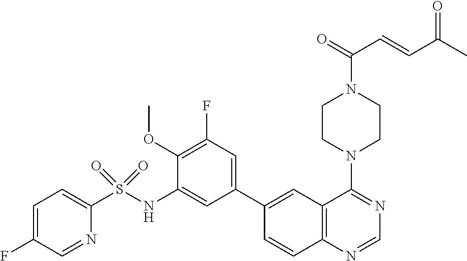 [SMILES: COc1c(F)cc(-c2ccc3ncnc(N4CCN(C(=O)/C=C/C(C)=O)CC4)c3c2)cc1NS(=O)(=O)c1ccc(F)cn1] | ¹H NMR (400 MHz, DMSO-d₆) δ 10.39 (s, 1H), 8.84 (s, 1H), 8.79 (s, 1H), 8.24 (s, 1H), 8.22-8.20 (m, 1H), 8.17-8.10 (m, 1H), 8.04-7.94 (m, 1H), 7.92 (d, J = 4.0 Hz, 1H), 7.64-7.61 (m, 2H), 7.42 (d, J = 16.0 Hz, 1H), 6.76 (d, J = 16.0 Hz, 1H), 4.25-4.20 (m, 4H), 4.19-3.93 (m, 2H), 3.84-3.81 (m. 2H), 3.66 (s, 3H), 2.36 (s, 3H); ESI-MS(m/z): 609.0 [M + H]⁺. |

### Example 122

### Preparation of (E)-2-cyano-6-fluoro-N-(2-methoxy-5-(4-(4-(4-oxopent-2-enoyl)piperazin-1-yl)quinazolin-6-yl)pyridin-3-yl)benzenesulfonamide

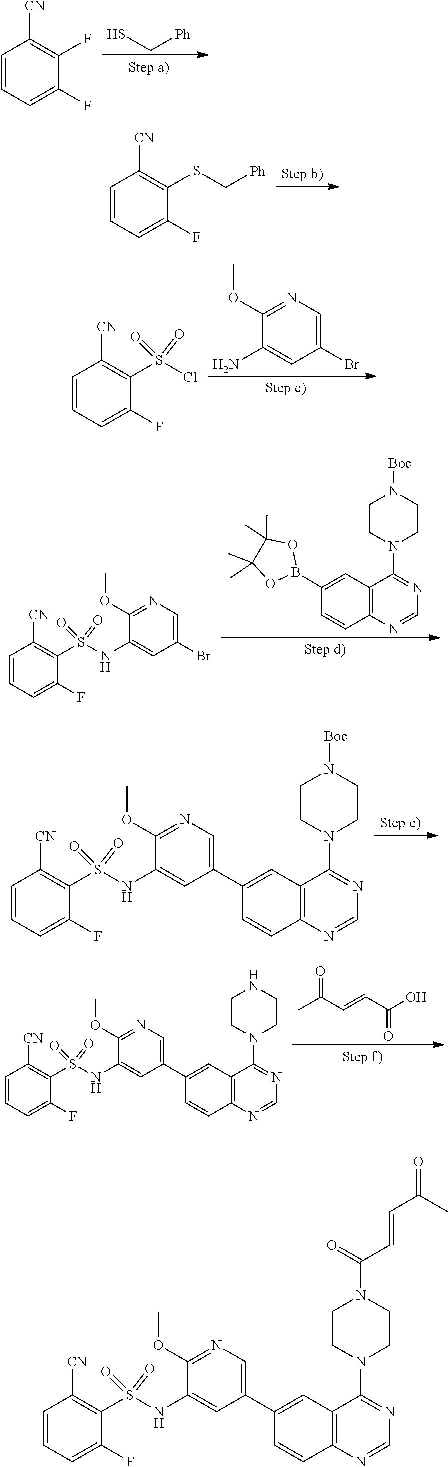

Referring to the preparation method of Example 118. (E)-2-cyano-6-fluoro-N-(2-methoxy-5-(4-(4-(4-oxopent-2-enoyl)piperazin-1-yl)quinazolin-6-yl)pyridin-3-yl)benzenesulfonamide was obtained, yield: 9.3%; ¹HNMR (400 MHz, DMSO-d₆) δ 10.89 (s, 1H), 8.68 (s, 1H), 8.42-8.36 (m, 1H), 8.15-8.06 (m, 3H), 7.93-7.82 (m, 4H), 7.46 (d, J=16 Hz, 1H), 6.73 (d, J=16 Hz, 1H), 3.94-3.90 (m, 6H), 3.88-3.81 (m, 2H), 3.59 (s, 3H), 2.37 (s, 3H); ESI-MS(m/z): 616.1 [M+H]⁺.

### Example 123

### Preparation of (E)-4-cyano-2-fluoro-N-(2-methoxy-5-(4-(4-(4-oxopent-2-enoyl)piperazin-1-yl)quinazolin-6-yl)pyridin-3-yl)benzenesulfonamide

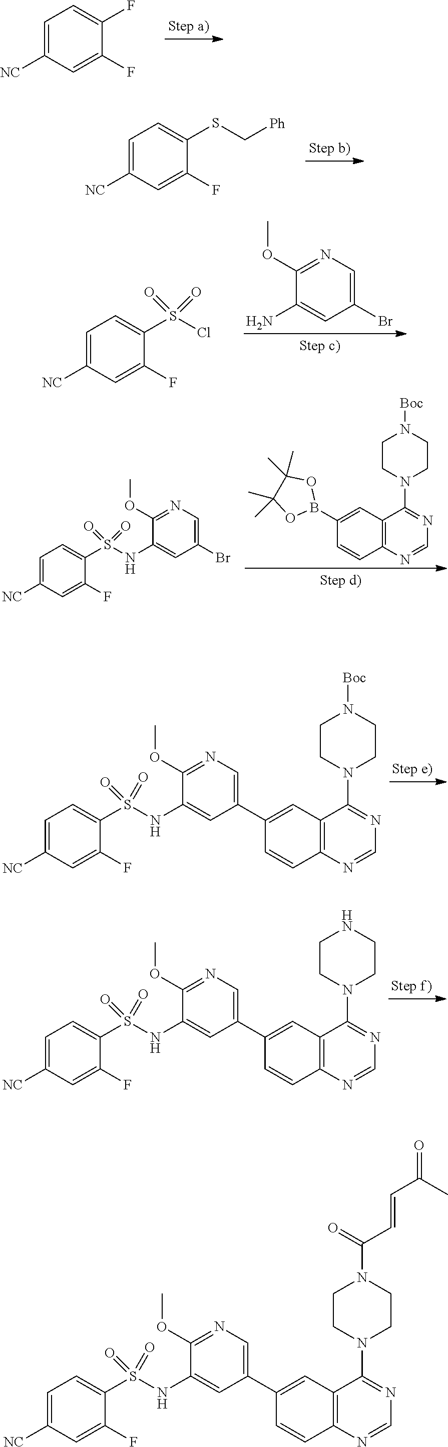

Referring to the preparation method of Example 118, (E)-4-cyano-2-fluoro-N-(2-methoxy-5-(4-(4-(4-oxopent-2-enoyl)piperazin-1-yl)quinazolin-6-yl)pyridin-3-yl)benzenesulfonamide was obtained, yield: 14.0% ¹H NMR (400 MHz, DMSO-d₆) δ 10.67 (s, 1H), 8.67 (s, 1H), 8.52-8.50 (m, 1H), 8.20-8.10 (m, 3H), 8.06 (d, J=8.0 Hz, 1H), 7.93-7.89 (m, 1H), 7.88-7.82 (m, 2H), 7.45 (d, J=16.0 Hz, 1H), 6.74 (d, J=16.0 Hz, 1H), 3.94-3.88 (m, 6H), 3.83-3.78 (m, 2H), 3.61 (s, 3H), 2.37 (s, 3H); ESI-MS(m/z): 616.2 [M+H]⁺.

### Example 124

### Preparation of (E)-2-fluoro-N-(2-methoxy-5-(4-(4-(4-oxopent-2-enoyl)piperazin-1-yl)quinazolin-4-yl)pyridin-3-yl)-4-(trifluoromethyl)benzenesulfonamide

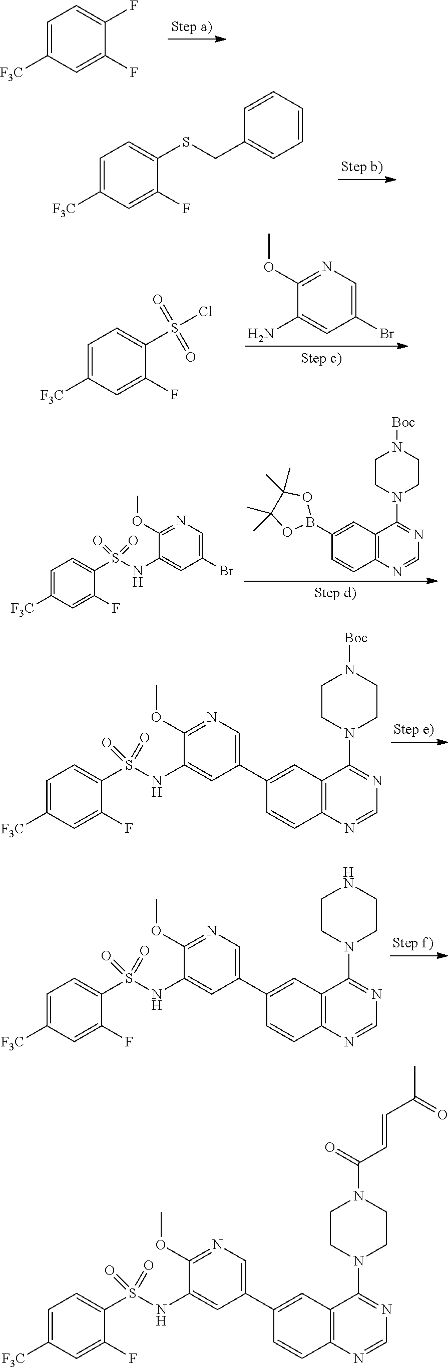

Referring to the preparation method of Example 118. (E)-2-fluoro-N-(2-methoxy-5-(4-(4-(4-oxopent-2-enoyl)piperazin-1-yl)quinazolin-6-yl)pyridin-3-yl)-4-(trifluoromethyl)benzenesulfonamide was obtained, yield: 10.9%; ¹H NMR (400 MHz, DMSO-d₆) δ 8.61 (s, 1H), 8.27 (s, 1H), 8.05-7.99 (m, 2H), 7.91-7.84 (m, 4H), 7.65 (d, J=8.0 Hz, 1H), 7.40 (d, J=16.0 Hz, 1H), 6.79 (d, J=12.0 Hz, 1H), 3.84-3.74 (m, 8H), 3.59 (s, 3H), 2.32 (m, 3H); ESI-MS(m/z): 659.0 [M+H]⁺.

### Example 125

### Preparation of 2,4-difluoro-N-(2-methoxy-5-(4-(3-(3-methylene-2-oxopyrrolidin-1-yl))azetidine-1-yl)quinazolin-6-yl)pyridin-3-yl)benzenesulfonamide

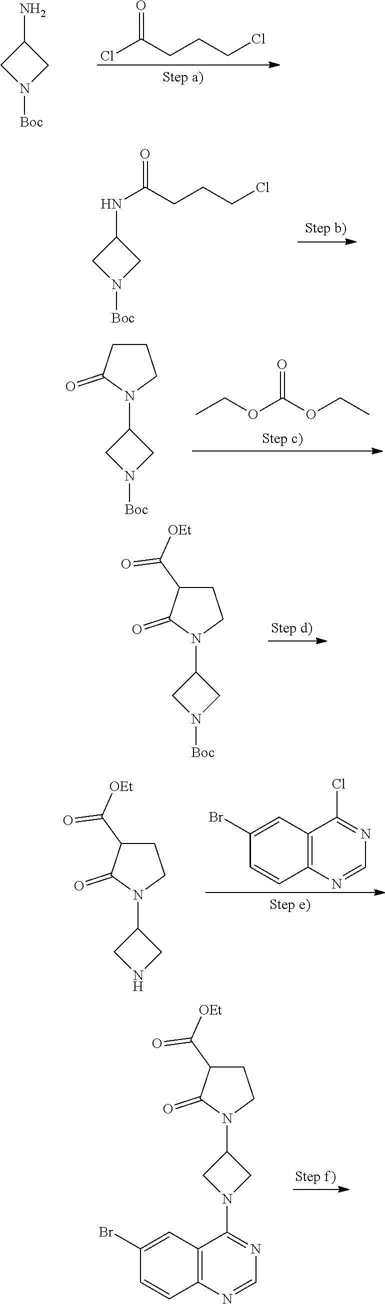

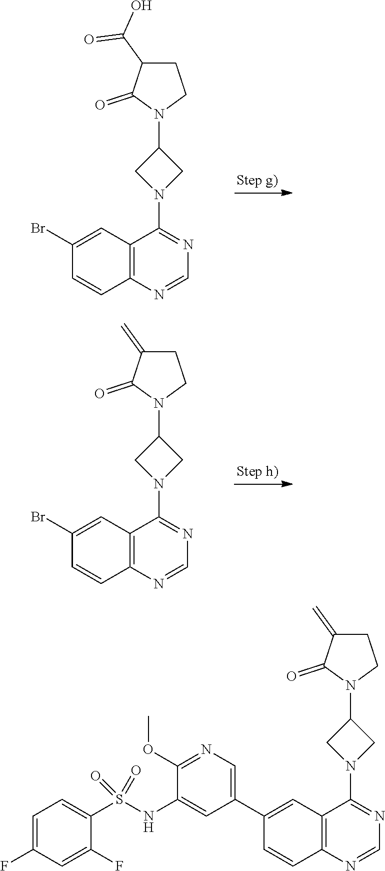

### Step a): Preparation of tert-butyl 3-(4-chlorobutanamido)azetidine-1-carboxylate

Tert-butyl 3-aminoazetidine-1-carboxylate (2.0 g, 11.627 mmol) and triethylamine (3.5 g, 34.881 mmol) were dissolved in dichloromethane (30 mL), with 4-chlorobutyryl chloride (2.4 g, 17.441 mmol) added. After the addition was completed, the reaction mixture was kept at room temperature and reacted overnight. Upon completion of the reaction, the reaction was quenched by adding water (50 mL). The reaction mixture was extracted with ethyl acetate (100 mL×2 times). The organic phases were combined, dried over anhydrous sodium sulfate and filtered. The filtrate was concentrated under reduced pressure. The crude product obtained was purified by silica gel column chromatography (Eluent: dichloromethane/methanol=30/1 to 10/1). Tert-butyl 3-(4-chlorobutanamido)azetidine-1-carboxylate was obtained, yield: 31.1%.

### Step b): Preparation of tert-butyl 3-(2-oxopyrrolidin-1-yl)azetidine-1-carboxylate

Tert-butyl 3-(4-chlorobutanamido)azetidine-1-carboxylate (950 mg, 3.430 mmol) was dissolved in tetrahydrofuran (15 mL), with sodium hydride (206 mg, 5.145 mmol) added at 0° C. After the addition was completed, the reaction mixture was slowly heated to room temperature and reacted overnight. Upon completion of the reaction, the reaction was quenched by adding saturated ammonium chloride solution (50 mL). The reaction mixture was extracted with ethyl acetate (100 mL×2 times). The organic phases were combined, washed with saturated brine (100 mL), dried over anhydrous sodium sulfate and filtered. The filtrate was concentrated under reduced pressure. The crude product obtained was purified by chromatography plate (Eluent: petroleum ether/ethyl acetate=1/1). Tert-butyl 3-(2-oxopyrrolidin-1-yl)azetidine-1-carboxylate was obtained, yield: 97.2%.

### Step c): Preparation of ethyl 1-(1-(tert-butoxycarbonyl)azetidin-3-yl)-2-oxopyrrolidine-3-carboxylate

Tert-butyl 3-(2-oxopyrrolidin-1-yl)azetidine-1-carboxylate (500 mg, 2.083 mmol) was dissolved in tetrahydrofuran (12 mL). At −78° C. lithium hexamethyldisilazide (4.2 mL, 4.166 mmol) was slowly added dropwise. After the addition was completed, the reaction mixture was kept at −78° C. and stirred for 1 h. Tetrahydrofuran (3 mL) solution of diethyl carbonate (492 mg, 4.166 mmol) was then slowly added dropwise. After the addition was completed, the reaction mixture was kept at −78° C. and stirred for 1 h, then slowly heated to 0° C. and stirred for 30 min. Upon completion of the reaction, the reaction was quenched by adding saturated ammonium chloride solution (50 mL). The reaction mixture was extracted with ethyl acetate (100 mL×2 times). The organic phases were combined, washed with saturated brine (100 mL), dried over anhydrous sodium sulfate and filtered. The filtrate was concentrated under reduced pressure. Ethyl 1-(1-(tert-butoxycarbonyl)azetidin-3-yl)-2-oxopyrrolidine-3-carboxylate was obtained, yield: 76.9%.

### Step d): Preparation of Ethyl 1-(azetidin-3-yl)-2-oxopyrrolidine-3-carboxylate

Ethyl 1-(1-(tert-butoxycarbonyl)azetidin-3-yl)-2-oxopyrrolidine-3-carboxylate (950 mg, 3.430 mmol) was dissolved in dichloromethane (5 mL). At 0° C. HCl dioxane solution (5 mL) was added to the system. After the addition was completed, the reaction mixture was slowly heated to room temperature and reacted for 2 h. Upon completion of the reaction, the reaction mixture was concentrated under reduced pressure. Ethyl 1-(azetidin-3-yl)-2-oxopyrrolidine-3-carboxylate was obtained.

### Step e): Preparation of ethyl 1-(1-(6-bromoquinazolin-4-yl)azetidin-3-yl)-2-oxopyrrolidine-3-carboxylate

Ethyl 1-(azetidin-3-yl)-2-oxopyrrolidine-3-carboxylate (400 mg, 1.887 mmol) and N,N-diisopropylethylamine (1.2 g, 9.435 mmol) were dissolved in N,N-dimethylformamide (30 mL), with 6-bromo-4-chloroquinazoline (688 mg, 2.831 mmol) added. After the addition was completed, the reaction mixture was kept at 60° C. and reacted for 3 h. Upon completion of the reaction, the reaction was quenched by adding water (50 mL). The reaction mixture was extracted with ethyl acetate (100 mL×2 times). The organic phases were combined, dried over anhydrous sodium sulfate and filtered. The filtrate was concentrated under reduced pressure. The crude product obtained was purified by silica gel column chromatography (Eluent: petroleum ether/ethyl acetate=2/1 to 1/2). Ethyl 1-(1-(6-bromoquinazolin-4-yl)azetidin-3-yl)-2-oxopyrrolidine-3-carboxylate was obtained, yield: 63.2%; ESI-MS(m/z): 419.1 [M+H]⁺.

### Step f): Preparation of 1-(1-(6-bromoquinazolin-4-yl)azetidin-3-yl)-2-oxopyrrolidine-3-carboxylic acid

Ethyl 1-(1-(6-bromoquinazolin-4-yl)azetidin-3-yl)-2-oxopyrrolidine-3-carboxylate (400 mg, 1.887 mmol) and lithium hydroxide (501.2 mg, 18.870 mmol) were dissolved in tetrahydrofuran/methanol/water mixed solvent (4:1:1, 10 mL). After the addition was completed, the reaction mixture was kept at room temperature and reacted for 3 h. Upon completion of the reaction, the pH of the reaction solution was adjusted to 3-4 by adding 2 M hydrogen chloride aqueous solution and extracted with dichloromethane (100 mL×2 times). The organic phases were combined, dried over anhydrous sodium sulfate and filtered. The filtrate was concentrated under reduced pressure. The crude product obtained was purified by silica gel column chromatography (Eluent: dichloromethane/methanol=20/1 to 10/1), 1-(1-(6-Bromoquinazolin-4-yl)azetidin-3-yl)-2-oxopyrrolidine-3-carboxylic acid was obtained, yield: 27.9%; ESI-MS(m/z): 391.2 [M+H]⁺.

### Step g): Preparation of 1-(1-(6-bromoquinazolin-4-yl)azetidin-3-yl)-3-methylenepyrrolidin-2-one

1-(1-(6-Bromoquinazolin-4-yl)azetidin-3-yl)-2-oxopyrrolidine-3-carboxylic acid (100 mg, 0.256 mmol) and paraformaldehyde (34.5 mg, 0.384 mmol) were dissolved in ethyl acetate (5 mL), with dimethylamine tetrahydrofuran solution (0.1 mL, 0.184 mmol) added at 0° C. After the addition was completed, the reaction mixture was kept at 80° C. and reacted for 3 h. Upon completion of the reaction, the reaction was quenched by adding water (50 mL). The reaction mixture was extracted with ethyl acetate (100 mL×2 times). The organic phases were combined, dried over anhydrous sodium sulfate and filtered. The filtrate was concentrated under reduced pressure. The crude product obtained was purified by silica gel column chromatography (Eluent: petroleum ether/ethyl acetate=2/1 to 1/2), 1-(1-(6-Bromoquinazolin-4-yl)azetidin-3-yl)-3-methylenepyrrolidin-2-one was obtained, yield: 43.5%; ESI-MS(m/z): 359.3 [M+H]⁺.

### Step h): Preparation of 2,4-difluoro-N-(2-methoxy-5-(4-(3-(3-methylene-2-oxopyrrolidin-1-yl))azetidine-1-yl)quinazolin-6-yl)pyridin-3-yl)benzenesulfonamide

1-(1-(6-Bromoquinazolin-4-yl)azetidin-3-yl)-3-methylenepyrrolidin-2-one (40 mg, 0.111 mmol), 2,4-difluoro-N-(2-hydroxy-5-(4,4,5,5-tetramethyl-1,3,2-dioxaborol-2-yl)pyridin-3-yl)benzene sulfonamide (57.0 mg, 0.133 mmol), Pd(dppf)Cl₂ (8.2 mg, 0.011 mmol) and cesium carbonate (109.0 mg, 0.333 mmol) were dissolved in dioxane/water (4:1, 10 mL). After the addition was completed, the reaction mixture was heated to 110° C. and stirred for 3 h. Upon completion of the reaction, the reaction was quenched by adding water (50 mL). The reaction mixture was extracted with ethyl acetate (100 mL×2 times). The organic phases were combined, washed with saturated brine (100 mL), dried over anhydrous sodium sulfate and filtered. The filtrate was concentrated under reduced pressure. The crude product obtained was purified by Prep-HPLC (Method 2), 2,4-difluoro-N-(2-methoxy-5-(4-(3-(3-methylene-2-oxopyrrolidin-1-yl))azetidine-1-yl)quinazolin-6-yl)pyridin-3-yl)benzenesulfonamide was obtained, yield: 27.7%; ¹H NMR (400 MHz, DMSO-d₆) δ 10.31 (s, 1H), 8.50 (s, 1H), 8.42 (s, 1H), 8.05-8.01 (m, 2H), 7.95 (s, 1H), 7.82-7.76 (m, 2H), 7.56-7.53 (m, 1H), 7.19-7.16 (m, 1H), 5.79 (s, 1H), 5.38 (s, 1H), 5.18-5.15 (m, 1H), 4.85-4.75 (m, 4H), 3.73-3.67 (m, 5H), 2.80-2.77 (m, 2H); ESI-MS(m/z): 579.0 [M+H]⁺.

### Example 126

### Preparation of 1-(4-(6-(5-((2,4-difluorobenzyl)amino)-6-methoxypyridin-3-yl)quinazolin-4-yl)piperazin-1-yl)prop-2-en-1-one

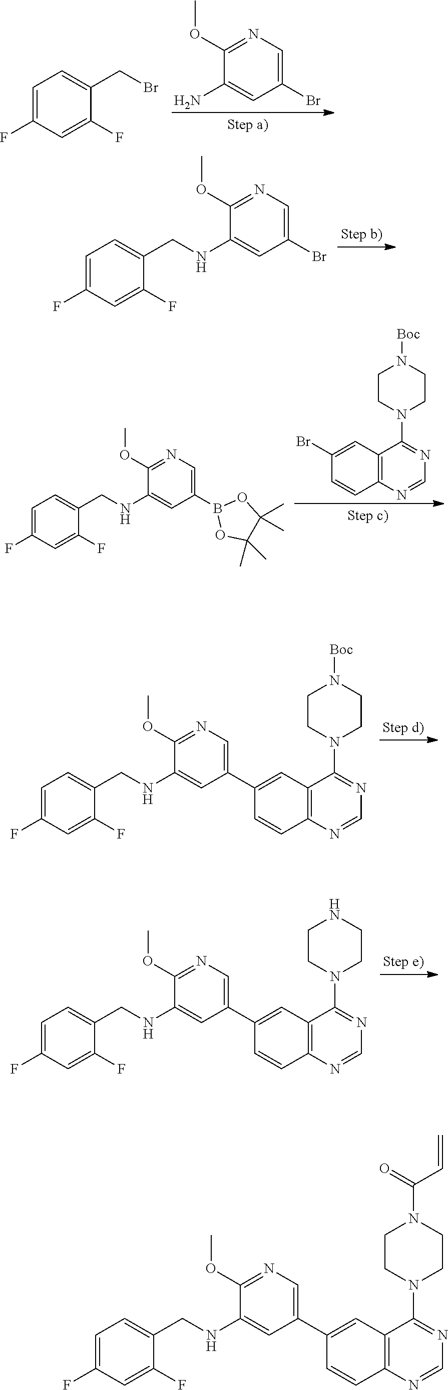

### Step a): Preparation of 5-bromo-N-(2,4-difluorobenzyl)-2-methoxypyridin-3-amine

1-(Bromomethyl)-2,4-difluorobenzene (500 mg, 2.415 mmol), 5-bromo-2-methoxypyridin-3-amine (491 mg, 2.419 mmol) and potassium carbonate (668 mg, 4.833 mmol) were added to N,N-dimethylformamide (5 mL). After the addition was completed, the reaction mixture was heated to 50° C. and stirred for 2 h. Upon completion of the reaction, the reaction was quenched by adding water (50 mL). The reaction mixture was extracted with ethyl acetate (60 mL/2 times). The organic phases were combined, washed with saturated brine (50 mL), dried over anhydrous sodium sulfate and filtered. The filtrate was concentrated under reduced pressure. The crude product obtained was purified by silica gel column chromatography (Eluent: petroleum ether/ethyl acetate=5/1 to 2/1), 5-Bromo-N-(2,4-difluorobenzyl)-2-methoxypyridin-3-amine was obtained, yield: 75.5%; ESI-MS(m/z): 329.0 [M+H]⁺.

### Step b): Preparation of N-(2,4-difluorobenzyl)-2-methoxy-5-(4,4,5,5-tetramethyl-1,3,2-dioxaborolan-2-yl)pyridin-3-amine

5-Bromo-N-(2,4-difluorobenzyl)-2-methoxypyridin-3-amine (600 mg, 1.823 mmol), pinacol diborate (695 mg, 2.737 mmol), Pd(dppf)Cl₂ (134 mg, 0.183 mmol) and potassium acetate (358 mg, 3.648 mmol) were added to dioxane (5 mL). After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (100 mL×2 times). The organic phases were combined, washed with saturated brine (100 mL), dried over anhydrous sodium sulfate and filtered. The filtrate was concentrated under reduced pressure. The crude product obtained was purified by silica gel column chromatography (Eluent: petroleum ether/ethyl acetate=2/1 to 1/1). N-(2,4-difluorobenzyl)-2-methoxy-5-(4,4,5,5-tetramethyl-1,3,2-dioxaborolan-2-yl)pyridin-3-amine was obtained, yield: 87.5%; ESI-MS(m/z): 377.2 [M+H]⁺.

### Step c): Preparation of tert-butyl 4-(6-(5-((2,4-difluorobenzyl)amino)-6-methoxypyridin-3-yl)quinazolin-4-yl)piperazine-1-carboxylate

N-(2,4-difluorobenzyl)-2-methoxy-5-(4,4,5,5-tetramethyl-1,3,2-dioxaborolan-2-yl)pyridin-3-amine (600 mg, 1.595 mmol), tert-butyl 4-(6-bromoquinazolin-4-yl)piperazine-1-carboxylate (690 mg, 1.754 mmol), Pd(dppf)Cl₂ (117 mg, 0.160 mmol) and cesium carbonate (1.0 g, 3.069 mmol) were added to dioxane/water mixed solvent (10 mL, v/v=5:1). After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (100 mL×2 times). The organic phases were combined, washed with saturated brine (100 mL), dried over anhydrous sodium sulfate and filtered. The filtrate was concentrated under reduced pressure. The crude product obtained was purified by silica gel column chromatography (Eluent: petroleum ether/ethyl acetate=2/1 to 1/2). Tert-butyl 4-(6-(5-((2,4-difluorobenzyl)amino)-6-methoxypyridin-3-yl)quinazolin-4-yl)piperazine-1-carboxylate was obtained, yield: 22.3%; ESI-MS(m/z): 563.3 [M+H]⁺.

### Step d): Preparation of N-(2,4-difluorobenzyl)-2-methoxy-5-(4-(piperazin-1-yl)quinazolin-6-yl)pyridin-3-amine-trifluoroacetate

Tert-butyl 4-(6-(5-((2,4-difluorobenzyl)amino)-6-methoxypyridin-3-yl)quinazolin-4-yl)piperazine-1-carboxylate (70 mg, 0.124 mmol) was dissolved in dichloromethane (2 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. Methyl tert-butyl ether was added to the crude product. The mixture was stirred to precipitate solid and filtered. N-(2,4-difluorobenzyl)-2-methoxy-5-(4-(piperazin-1-yl)quinazolin-6-yl)pyridin-3-amine-trifluoroacetate was obtained, yield: 98.5%. ESI-MS(m/z): 463.2 [M+H]⁺.

### Step e): Preparation of 1-(4-(6-(5-((2,4-difluorobenzyl)amino)-6-methoxypyridin-3-yl)quinazolin-4-yl)piperazin-1-yl)prop-2-en-1-one

Acrylic acid (21 mg, 0.291 mmol) and HATU (44 mg, 0.116 mmol) were dissolved in DMF (2 mL). With the reaction system cooled to −41° C., N,N-diisopropylethylamine (62 mg, 0.480 mmol) and N-(2,4-difluorobenzyl)-2-methoxy-5-(4-(piperazin-1-yl)quinazolin-6-yl)pyridin-3-amine-trifluoroacetate (55 mg, 0.095 mmol) were added successively. After the addition was completed, the reaction mixture was kept at −41° C. and stirred for 1 h. Upon completion of the reaction, the reaction solution was purified by Prep-HPLC (Method 2), 1-(4-(6-(5-((2,4-Difluorobenzyl)amino)-6-methoxypyridin-3-yl)quinazolin-4-yl)piperazin-1-yl)prop-2-en-1-one was obtained, yield: 12.1%; ¹H NMR (400 MHz, DMSO-d₆) δ 8.63 (s, 1H), 8.05-7.97 (m, 2H), 7.88-7.79 (m, 2H), 7.46-7.39 (m, 1H), 7.23-7.15 (m, 1H), 7.07-6.99 (m, 2H), 6.86-6.78 (m, 1H), 6.21-6.14 (dd, J₁=16.0 Hz, J=4.0 Hz, 1H), 6.11-5.92 (m, 1H), 5.76-5.72 (dd, J₁=12.0 Hz, J₂=4.0 Hz, 1H), 4.51-4.46 (m, 2H), 3.97 (s, 3H), 3.83-3.73 (m, 8H); ESI-MS(m/z): 517.0 [M+H]⁺.

### Example 127

### Preparation of N-(5-(4-(4-(3-acetyloxirane-2-carbonyl)piperazin-1-yl)quinazolin-6-yl)-2-methoxypyridin-3-yl)-2,4-difluorobenzenesulfonamide

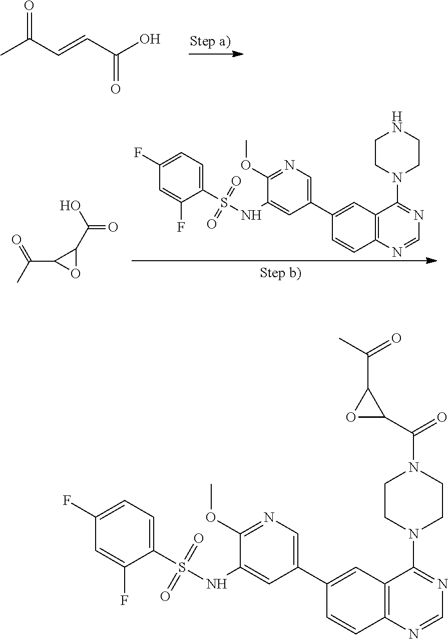

### Step a): Preparation of 3-acetyloxirane-2-carboxylic acid

(E)-4-oxopent-2-enoic acid (100 mg, 0.876 mmol), potassium carbonate (484 mg, 3.504 mmol) were added to tert-butanol (0.5 ml) and water (0.5 ml) mixed solvent. Under the ice bath condition, hydrogen peroxide (855 mg, 35% wt %) was slowly added to the system. After the addition was completed, the reaction mixture was kept at room temperature and reacted overnight. Upon completion of the reaction, the reaction mixture was adjusted to pH=1 with hydrochloric acid (2M) under the ice bath condition and extracted with ethyl acetate (50 mL×2 times). The organic phases were combined, washed with saturated brine (50 mL), dried over anhydrous sodium sulfate and filtered. The filtrate was concentrated under reduced pressure. 3-Acetyloxirane-2-carboxylic acid was obtained, yield: 37.3%. ESI-MS(m/z): 129.0 [M−H]⁻.

### Step b): Preparation of N-(5-(4-(4-(3-acetyloxirane-2-carbonyl)piperazin-1-yl)quinazolin-6-yl)-2-methoxypyridin-3-yl)-2,4-difluorobenzenesulfonamide

2,4-difluoro-N-(2-methoxy-5-(4-(piperazin-1-yl)quinazolin-6-yl)pyridin-3-yl)benzenesulfonamide trifluoroacetate (80 mg, 0.128 mmol) was dissolved in tetrahydrofuran (2 mL). The reaction system was cooled to −78° C., with N,N-diisopropylethylamine (99 mg, 0.768 mmol), 3-acetyloxirane-2-carboxylic acid (17 mg, 0.128 mmol) and 50% T₃P ethyl acetate solution (163 mg, 0.256 mmol) added successively. After the addition was completed, the reaction mixture was kept at −78° C. and stirred for 1 h. Upon completion of the reaction, the reaction solution was diluted by adding acetonitrile (1 mL) and purified by Prep-HPLC (Method 2). N-(5-(4-(4-(3-acetyloxirane-2-carbonyl)piperazin-1-yl)quinazolin-6-yl)-2-methoxypyridin-3-yl)-2,4-difluorobenzenesulfonamide was obtained, yield: 25.1%; ¹H NMR (400 MHz, DMSO-d₆) δ 10.37 (brs, 1H), 8.75 (s, 1H), 8.50 (d, J=8.0 Hz, 1H), 8.20-8.16 (m, 2H), 8.04 (d, J=8.0 Hz, 1H), 7.93 (d, J=8.0 Hz, 1H), 7.80-7.74 (m, 1H), 7.62-7.56 (m, 1H), 7.25-7.20 (m, 1H), 4.26 (d, J=8.0 Hz, 1H), 4.05-3.72 (m, 8H), 3.67 (s, 3H), 3.65 (d, J=8.0 Hz, 1H), 2.13 (s, 3H); ESI-MS(m/z): 625.0 [M+H]⁺.

### Example 128

### N-(2,4-difluorophenyl)[(2-methoxy-5-(4-(4-[(2E)-4-oxopent-2-enoyl]piperazin-1-yl)quinazolin-6-yl)pyridin-3-yl)amino]sulfonamide

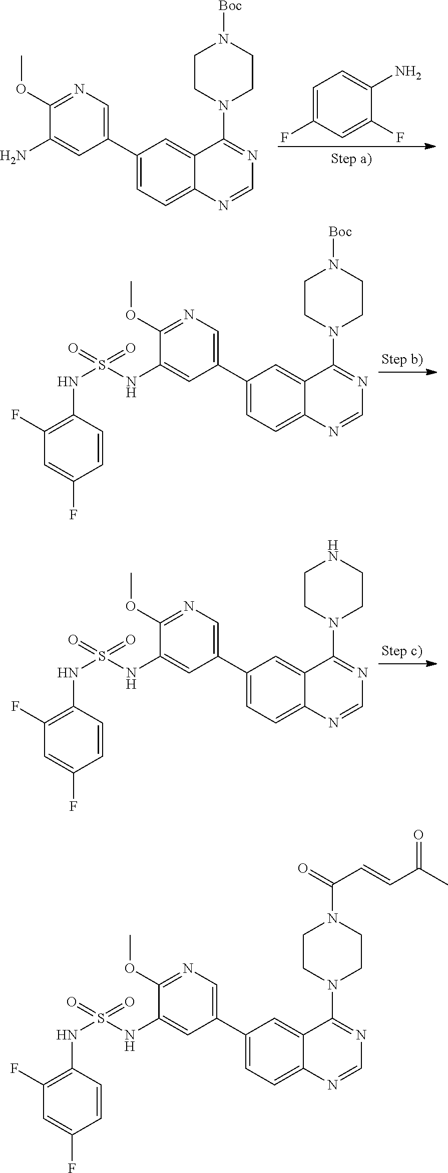

### Step a): Preparation of tert-butyl 4-(6-(5-((N-(2,4-difluorophenyl)sulfamoyl)amino)-6-methoxypyridin-3-yl)quinazolin-4-yl)piperazine-1-carboxylate

2,4-Difluoroaniline (130 mg, 1.007 mmol), triethylamine (408 mg, 4.028 mmol) were added to dichloromethane (5 mL). At −78° C., sulfonyl chloride (136 mg, 1.007 mmol) was added to the reaction solution dropwise. After the addition was completed, the reaction mixture was heated to room temperature and reacted for 5 h. At the same time, tert-butyl 4-(6-(5-amino-6-methoxypyridin-3-yl)quinazolin-4-yl)piperazine-1-carboxylate (132 mg, 0.302 mmol), pyridine (72 mg, 0.906 mmol), 4-dimethylaminopyridine (12 mg, 0.100 mmol) and dichloromethane (5 mL) were added to another reaction flask. After the solution was stirred to dissolve, the sulfonyl chloride reaction solution was added to the newly prepared solution. After the addition was completed, the reaction mixture was kept at room temperature and reacted overnight. Upon completion of the reaction, the reaction was quenched by adding water (50 mL). The reaction mixture was extracted with ethyl acetate (100 mL×2 times). The organic phases were combined, washed with saturated brine (50 mL), dried over anhydrous sodium sulfate, concentrated under reduced pressure. The residue was purified by silica gel column chromatography (Eluent: ethyl acetate/petroleum ether=1/10 to 1/1). Tert-butyl 4-(6-(5-((N-(2,4-difluorophenyl)sulfamoyl)amino)-6-methoxypyridin-3-yl)quinazolin-4-yl)piperazine-1-carboxylate was obtained, yield: 15.3%; ESI-MS(m/z): 628.2 [M+H]⁺.

### Step b): Preparation of N-(2,4-difluorophenyl)[(2-methoxy-5-(4-(piperazin-1-yl)quinazolin-6-yl)pyridin-3-yl)amino]sulfonateamide trifluoroacetate

Tert-butyl 4-(6-(5-((N-(2,4-difluorophenyl)sulfamoyl)amino)-6-methoxypyridin-3-yl)quinazolin-4-yl)piperazine-1-carboxylate (90 mg, 0.143 mmol) was dissolved in dichloromethane (3 mL). Under the ice bath condition, TFA (1 mL) was slowly added dropwise. After the addition was completed, the reaction mixture was stirred at room temperature for 1 h. Upon completion of the reaction, the reaction mixture was concentrated under reduced pressure. The crude product was slurried with methyl tert-butyl ether (5 mL) to precipitate solid and filtered. N-(2,4-difluorophenyl)[(2-methoxy-5-(4-(piperazin-1-yl)quinazolin-6-yl)pyridin-3-yl)amino]sulfonateamide trifluoroacetate was obtained, yield: 92.5%; ESI-MS(m/z): 528.2 [M+H]⁺.

### Step c): Preparation of N-(2,4-difluorophenyl)[(2-methoxy-5-(4-(4-[(2E)-4-oxopent-2-enoyl]piperazin-1-yl)quinazolin-6-yl)pyridin-3-yl)amino]sulfonamide

Referring to the preparation method of Example 109 Step e, N-(2,4-difluorophenyl)[(2-methoxy-5-(4-(4-[(2E)-4-oxopent-2-enoyl]piperazin-1-yl)quinazolin-6-yl)pyridin-3-yl)amino]sulfonamide was obtained, yield: 22.2%; ¹H NMR (400 MHz, DMSO-d₆) δ 10.07 (brs, 1H), 9.65 (brs, 1H), 8.67 (s, 1H), 8.37 (d, J=4.0 Hz, 1H), 8.17-8.11 (m, 3H), 7.94 (d, J=16.0 Hz, 1H), 7.54-7.48 (m, 1H), 7.44 (d, J=16.0 Hz, 1H), 7.30-7.24 (m, 1H), 7.13-7.07 (m, 1H), 6.74 (d, J=16.0 Hz, 1H), 3.91-3.88 (m, 5H), 3.87 (s, 3H), 3.81-3.80 (m, 3H), 2.37 (s, 3H); ESI-MS(m/z): 624.0 [M+H]⁺.

### Example 129

### Preparation of (E)-2,4-difluoro-N-(2-hydroxy-5-(4-(4-(4-oxopent-2-enoyl)piperazin-1-yl)quinazolin-6-yl)pyridin-3-yl)benzenesulfonamide

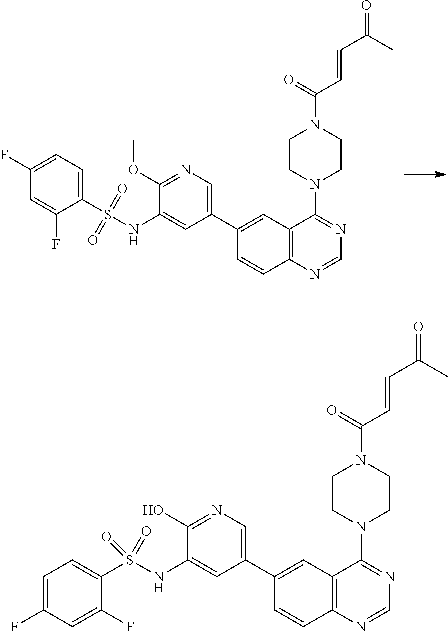 [SMILES: COc1ncc(-c2ccc3ncnc(N4CCN(C(=O)/C=C/C(C)=O)CC4)c3c2)cc1NS(=O)(=O)c1ccc(F)cc1F ; CC(=O)/C=C/C(=O)N1CCN(c2ncnc3ccc(-c4cnc(O)c(NS(=O)(=O)c5ccc(F)cc5F)c4)cc23)CC1]

(E)-2,4-difluoro-N-(2-methoxy-5-(4-(4-(4-oxopent-2-enoyl)piperazin-1-yl)quinazolin-6-yl)pyridin-3-yl)benzenesulfonamide (120 mg, 0.197 mmol) were dissolved in acetonitrile (1 mL) and distilled water (2 mL), with 2 M dilute hydrochloric acid (2 mL) added dropwise to the reaction system. After the addition was completed, the reaction solution was heated to 45° C., stirred for 2 h and freeze dried. The crude product obtained was purified by Prep-HPLC (Method 2). (E)-2,4-difluoro-N-(2-hydroxy-5-(4-(4-(4-oxopent-2-enoyl)piperazin-1-yl)quinazolin-6-yl)pyridin-3-yl)benzenesulfonamide was obtained, yield: 8.5%; ¹H NMR (400 MHz, DMSO-d₆) δ 12.26 (s, 1H), 9.86 (s, 1H), 8.65 (s, 1H), 8.02-7.97 (m, 2H), 7.96-7.89 (m, 1H), 7.88-7.85 (m, 1H), 7.85-7.71 (m, 2H), 7.54-7.48 (m, 1H), 7.45 (d, J=16.0 Hz, 1H), 7.27-7.20 (m, 1H), 6.73 (d, J=16.0 Hz, 1H), 3.89-3.84 (m, 6H), 3.82-3.78 (m, 2H), 2.37 (s, 3H); ESI-MS(m/z): 595.0 [M+H]⁺.

### Example 130

### Preparation of (E)-2,4-difluoro-N-(2-methoxy-5-(4-((1-(4-oxopent-2-enoyl)piperidin-4-yl)amino)quinazolin-6-yl)pyridin-3-yl)benzenesulfonamide

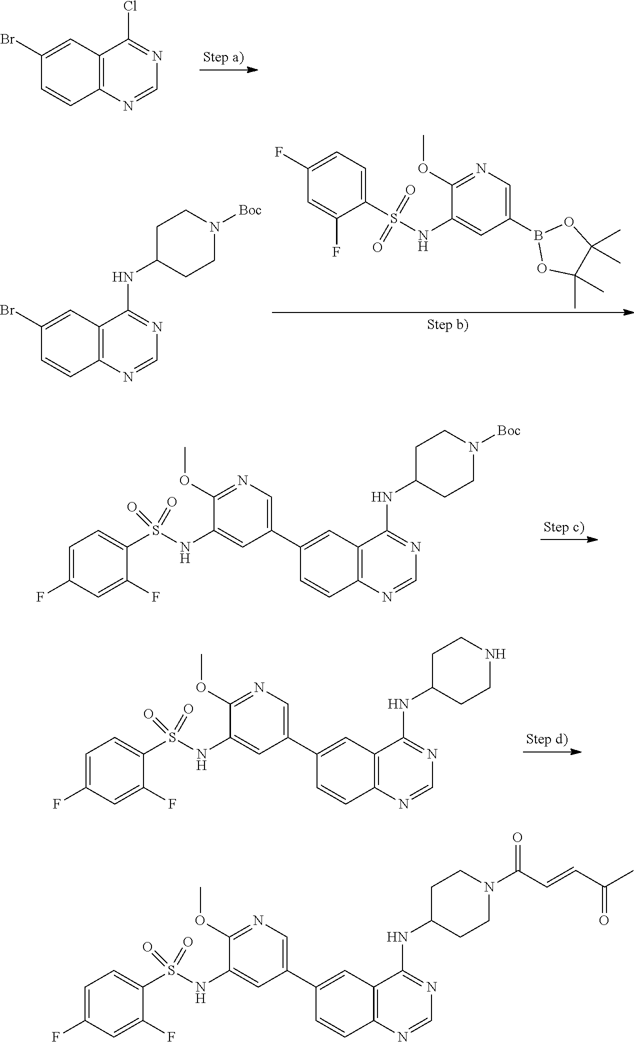 [SMILES: Clc1ncnc2ccc(Br)cc12 ; CC(C)(C)OC(=O)N1CCC(Nc2ncnc3ccc(Br)cc23)CC1 ; COc1ncc(B2OC(C)(C)C(C)(C)O2)cc1NS(=O)(=O)c1ccc(F)cc1F ; COc1ncc(-c2ccc3ncnc(NC4CCN(C(=O)OC(C)(C)C)CC4)c3c2)cc1NS(=O)(=O)c1ccc(F)cc1F ; COc1ncc(-c2ccc3ncnc(NC4CCNCC4)c3c2)cc1NS(=O)(=O)c1ccc(F)cc1F ; COc1ncc(-c2ccc3ncnc(NC4CCN(C(=O)/C=C/C(C)=O)CC4)c3c2)cc1NS(=O)(=O)c1ccc(F)cc1F]

### Step a): Preparation of tert-butyl 4-((6-bromoquinazolin-4-yl)amino)piperidine-1-carboxylate

6-Bromo-4-chloroquinazoline (500.0 mg, 2.053 mmol) and tert-butyl 4-aminopiperidine-1-carboxylate (492.8 mag, 2.464 mol) were added to N,N-dimethylformamide (25 mL). The reaction mixture was stirred at room temperature, with N,N-diisopropylethylamine (533.8 mg, 4.106 mmol) added. After the addition was completed, the reaction mixture was heated to 80° C. and stirred for 1 hi. Upon completion of the reaction, the reaction solution was cooled to room temperature, with water (100 mL) and ethyl acetate (100 mL) added, stirred for 10 mi. The liquids were separated and the organic phase was washed with water (100 mL×3), dried over anhydrous sodium sulfate and filtered. The filtrate was concentrated under reduced pressure. The residue was purified by silica gel column chromatography (Eluent: petroleum ether/ethyl acetate=3/1 to 1/1). Tert-butyl 4-((6-bromoquinazolin-4-yl)amino)piperidine-1-carboxylate was obtained, yield: 98.1%; ESI-MS (m/z): 407.1 [M+H]⁺.

### Step b): Preparation of tert-butyl 4-((6-(5-((2,4-difluorophenyl)sulfonamido)-6-methoxypyridin-3-yl)quinazolin-4-yl)amino)piperidine-1-carboxylate

Tert-buty 4-((6-bromoquinazolin-4-yl)amino)piperidine-1-carboxylate (400.0 mg, 0.982 mmol), 2,4-difluoro-N-(2-methoxy-5-(4,4,5,5-tetramethyl-1,3,2-dioxaborolan-2-yl)pyridin-3-yl)benzenesulfonamide (586.2 mg, 1.375 mmol), Pd(dppf)Cl₂ (115.0 mg, 0.098 mmol), cesium carbonate (640.5 mg, 1.963 mmol), 1,4-dioxane (5 mL) and water (i mL) were added to the reaction flask, with nitrogen purged three times, the reaction mixture was heated to 85° C., stirred and reacted for 2 h. Upon completion of the reaction, the reaction solution was concentrated under reduced pressure. The residue was purified by silica gel column chromatography (Eluent: petroleum ether/ethyl acetate=30/1 to 10/1). Tert-butyl 4-((6-(5-((2,4-difluorophenyl)sulfonamido)-6-methoxypyridin-3-yl)quinazolin-4-yl)amino)piperidine-1-carboxylate was obtained, yield 63.3%; ESI-MS (m/z): 627.2 [M+H]⁺.

### Step c): Preparation of 2,4-difluoro-N-(2-methoxy-5-(4-(piperidin-4-ylamino)quinazolin-6-yl)pyridin-3-yl)benzenesulfonamide

Tert-butyl 4-((6-(5-((2,4-difluorophenyl)sulfonamido)-6-methoxypyridin-3-yl)quinazolin-4-yl)amino)piperidine-1-carboxylate (200.0 mg, 0.380 mmol) was dissolved in dichloromethane (1 mL), with trifluoroacetic acid (mL) added. The reaction mixture was stirred at room temperature for 1 h, concentrated under reduced pressure, then with methyl tert-butyl ether (5 mL) added, stirred at room temperature for 30 mi and filtered. The filter cake was dried to obtain 2,4-difluoro-N-(2-methoxy-5-(4-(piperidin-4-ylamino)quinazolin-6-yl)pyridin-3-yl)benzenesulfonamide, yield: 98.4%; ESI-MS (m/z): 527.6 [M+H]⁺.

### Step d): Preparation of (E)-2,4-difluoro-N-(2-methoxy-5-(4-((1-(4-oxopent-2-enoyl)piperidin-4-yl)amino)quinazolin-6-yl)pyridin-3-yl)benzenesulfonamide

2,4-difluoro-N-(2-methoxy-5-(4-(piperidin-4-ylamino)quinazolin-6-yl)pyridin-3-yl)benzenesulfonamide (160.0 mg, 0.257 mmol), acetylacrylic acid (29.2 mg, 0.257 mmol), N,N-diisopropylethylamine (100.0 mg, 0.769 mmol) and tetrahydrofuran (2 mL) were added to the reaction flask. At −78° C., under the condition of stirring, 1-propylphosphonic anhydride (123.2 mg, 0.385 mmol) was slowly added to the system. After the addition was completed, the reaction mixture was kept at −78° C. and reacted for 30 min. Upon completion of the reaction, water (5 mL) and ethyl acetate (10 mL) were added to the reaction solution. The reaction solution was naturally heated to room temperature and stirred for 10 min. The liquids were separated and the organic phase was dried over anhydrous sodium sulfate and filtered. The filtrate was concentrated under reduced pressure. The residue was purified by Prep-HPLC (Method 2). (E)-2,4-difluoro-N-(2-methoxy-5-(4-((1-(4-oxopent-2-enoyl)piperidin-4-yl)amino)quinazolin-6-yl)pyridin-3-yl)benzenesulfonamide was obtained, yield: 39.8%; ¹H NMR (400 MHz, DMSO-dc) a 10.32 (s, 1H), 8.57-8.51 (m, 1H), 8.49 (s, 1H), 8.45-8.37 (m, 1H), 8.08 (d, J=8.0 Hz, 1H), 8.05-7.98 (m, 2H), 7.79-7.68 (m, 2H), 7.57-7.43 (m, 2H), 7.22-7.13 (m, 1H), 6.69 (d, J=16.0 Hz, 1H), 4.63-4.44 (m, 2H), 4.18 (d, J=14.0 Hz, 1H), 3.64 (s, 3H), 3.28 (s, 1H), 2.94-2.87 (m, 1H), 2.37 (s, 3H), 2.08 (s, 2H), 1.63-1.51 (m, 2H); ESI-MS (m/z): 623.2 [M+H]+.

### Example 131

### Preparation of (E)-2,6-difluoro-N-(2-methoxy-5-(4-(4-(4-oxopent-2-enoyl)piperazin-1-yl)pyrido[3,2-d]pyrimidin-6-yl)pyridin-3-yl)benzenesulfonamide

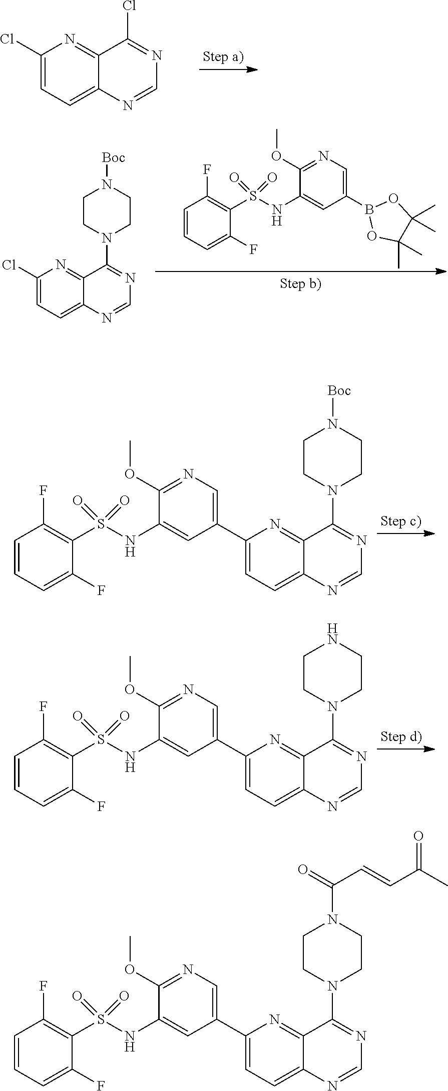 [SMILES: Clc1ccc2ncnc(Cl)c2n1 ; CC(C)(C)OC(=O)N1CCN(c2ncnc3ccc(Cl)nc23)CC1 ; COc1ncc(B2OC(C)(C)C(C)(C)O2)cc1NS(=O)(=O)c1c(F)cccc1F ; COc1ncc(-c2ccc3ncnc(N4CCN(C(=O)OC(C)(C)C)CC4)c3n2)cc1NS(=O)(=O)c1c(F)cccc1F ; COc1ncc(-c2ccc3ncnc(N4CCNCC4)c3n2)cc1NS(=O)(=O)c1c(F)cccc1F ; COc1ncc(-c2ccc3ncnc(N4CCN(C(=O)/C=C/C(C)=O)CC4)c3n2)cc1NS(=O)(=O)c1c(F)cccc1F]

### Step a): Preparation of tert-butyl 4-(6-chloropyrido[3,2-d]pyrimidin-4-yl)piperazine-1-carboxylate

4,6-Dichloropyrido[3,2-d]pyrimidine (250 mg, 1.250 mmol), tert-butyl piperazine-1-carboxylate (350 mg, 1.875 mmol), N,N-diisopropylethylamine (485 mg, 3.750 mmol) and N,N-dimethylformamide (5 mL) were added to the reaction flask. The reaction mixture was heated to 55° C. and reacted for 2 h. Upon completion of the reaction, the reaction solution was cooled to room temperature, with water (20 mL) and ethyl acetate (20 mL) added, stirred for 10 min. The liquids were separated and the organic phase was dried over anhydrous sodium sulfate and filtered. The filtrate was concentrated under reduced pressure. The residue was purified by silica gel column chromatography (Eluent: petroleum ether/ethyl acetate=1/1). Tert-butyl 4-(6-chloropyrido[3,2-d]pyrimidin-4-yl)piperazine-1-carboxylate was obtained, yield: 99.1%; ESI-MS(m/z): 350.0 [M+H]⁺.

### Step b): Preparation of tert-butyl 4-(6-(5-((2,6-difluorophenyl)sulfonamido)-6-methoxypyridin-3-yl)pyrido[3,2-d]pyrimidin-4-yl)piperazine-1-carboxylate

Tert-butyl 4-(6-chloropyrido[3,2-d]pyrimidin-4-yl)piperazine-1-carboxylate (430 mg, 1.230 mmol), 2,6-difluoro-N-(2-methoxy-5-(4,4,5,5-tetramethyl-1,3,2-dioxaborolan-2-yl)pyridin-3-yl)benzenesulfonamide (550 mg, 1.290 mmol), Pd(dppf)Cl₂ (47 mg, 0.065 mmol), cesium carbonate (840 mg, 2.50 mmol), 1,4-dioxane (10 mL) and water (1.5 mL) were added to the reaction flask. With nitrogen purged three times, in the presense of protective nitrogen, the reaction mixture was heated to 85° C. and stirred for 1 h. Upon completion of the reaction, the reaction solution was concentrated under reduced pressure. The residue was purified by silica gel column chromatography (Eluent: petroleum ether/ethyl acetate=1/3). Tert-butyl 4-(6-(5-((2,6-difluorophenyl)sulfonamido)-6-methoxypyridin-3-yl)pyrido[3,2-d]pyrimidin-4-yl)piperazine-1-carboxylate was obtained, yield: 95.2%; ESI-MS(m/z): 614.0[M+H]⁺.

### Step c): Preparation of 2,6-difluoro-N-(2-methoxy-5-(4-(piperazin-1-yl)pyrido[3,2-d]pyrimidin-6-yl)pyridin-3-yl)benzenesulfonamide trifluoroacetate

Tert-butyl 4-(6-(5-((2,6-difluorophenyl)sulfonamido)-6-methoxypyridin-3-yl)pyrido[3,2-d]pyrimidin-4-yl)piperazine-1-carboxylate (160 mg, 0.260 mmol) and dichloromethane (1 mL) were added to the reaction flask and stirred to mix. After that, with trifluoroacetic acid (1 mL) added, the reaction mixture was stirred at room temperature for 1 h, concentrated under reduced pressure, then with methyl tert-butyl ether (5 mL) added, stirred at room temperature for 30 min and filtered. The filter cake was vacuum dried to obtain 2,6-difluoro-N-(2-methoxy-5-(4-(piperazin-1-yl)pyrido[3,2-d]pyrimidin-6-yl)pyridin-3-yl)benzenesulfonamide trifluoroacetate, yield: 99.9%; ESI-MS (m/z): 514.2 [M+H]⁺.

### Step d): Preparation of (E)-2,6-difluoro-N-(2-methoxy-5-(4-(4-(4-oxopent-2-enoyl)piperazin-1-yl)pyrido[3,2-d]pyrimidin-6-yl)pyridin-3-yl)benzenesulfonamide

2,6-Difluoro-N-(2-methoxy-5-(4-(piperazin-1-yl)pyrido[3,2-d]pyrimidin-6-yl)pyridin-3-yl)benzenesulfonamide trifluoroacetate (220 mg, 0.260 mmol), (E)-acrylic acid (40 mg, 0.350 mmol) and tetrahydrofuran (2 mL) were added to the reaction flask. The reaction solution was kept at −78° C. and stirred for 10 min. Then N,N-diisopropylethylamine (189.8 mg, 1.460 mmol) was added. After that, the reaction solution was still kept at −78° C. and stirred for 10 min. After that, 50% 1-propylphosphonic anhydride (185.7 mg, 0.584 mmol) was slowly added to the system. After the addition was completed, the reaction solution was kept at −78° C. and reacted for 30 min. Upon completion of the reaction, the reaction system was quenched by adding water (10 mL). The reaction mixture was extracted with ethyl acetate (20 mL×4). The organic phases were combined, dried over anhydrous sodium sulfate and filtered. The filtrate was concentrated under reduced pressure. The residue was purified by Prep-HPLC (Method 2). (E)-2,6-difluoro-N-(2-methoxy-5-(4-(4-(4-oxopent-2-enoyl)piperazin-1-yl)pyrido[3,2-d]pyrimidin-6-yl)pyridin-3-yl)benzenesulfonamide was obtained, yield: 68.8%; ¹H NMR (400 MHz, DMSO) δ 8.56 (s, 1H), 8.55 (s, 1H), 8.39 (s, 2H), 8.18 (d, J=8.8 Hz, 1H), 7.56 (s, 1H), 7.46 (d, J=16.0 Hz, 1H), 7.15 (t, J=9.2 Hz, 3H), 6.71 (d, J=15.6 Hz, 1H), 4.56-4.43 (m, 4H), 3.94-3.91 (m, 2H), 3.82-3.79 (m, 2H), 3.75 (s, 3H), 2.33 (s, 3H); ESI-MS (m/z): 610.0 [M+H]⁺.

### Example 132

Preparation of (E)-2,4-difluoro-N-(2-methoxy-5-(4-(4-(4-oxopent-2-enoyl)piperazin-1-yl)pyrido[3,2-d]pyrimidin-6-yl)pyridin-3-yl)benzenesulfonamide

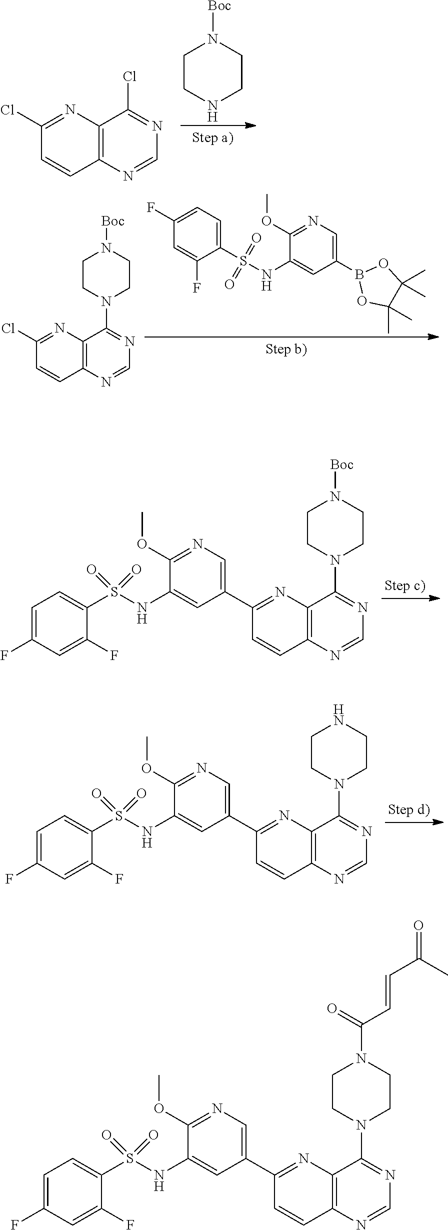

### Step a): Preparation of tert-butyl 4-(6-chloropyrido[3,2-d]pyrimidin-4-yl)piperazine-1-carboxylate

4,6-Dichloropyrido[3,2-d]pyrimidine (145 mg, 0.725 mmol), tert-butyl piperazine-1-carboxylate (135 mg, 0.725 mmol), diisopropylethylamine (281 mg, 2.175 mmol) were added to N,N-dimethylformamide (3 mL). The reaction mixture was kept at 60° C. and reacted for 2 h. Upon completion of the reaction, the reaction was quenched by adding water (30 mL). The reaction mixture was extracted with ethyl acetate (50 mL×2). The organic phases were combined, washed with saturated brine (30 mL), dried over anhydrous sodium sulfate and filtered. The filtrate was concentrated under reduced pressure. The crude product obtained was purified by silica gel column chromatography (Eluent: methanol/dichloromethane=1/100 to 1/50). Tert-butyl 4-(6-chloropyrido[3,2-d]pyrimidin-4-yl)piperazine-1-carboxylate was obtained, yield: 90.8%; ESI-MS(m/z): 350.1 [M+H]⁺.

### Step b): Preparation of tert-butyl 4-(6-(5-((2,4-difluorophenyl)sulfonamido)-6-methoxypyridin-3-yl)pyrido[3,2-d]pyrimidin-4-yl)piperazine-1-carboxylate

Tert-butyl 4-(6-chloropyrido[3,2-d]pyrimidin-4-yl)piperazine-1-carboxylate (130 mg, 0.372 mmol), 2,4-difluoro-N-(2-methoxy-5-(4,4,5,5-tetramethyl-1,3,2-dioxaborolan-2-yl)pyridin-3-yl)benzenesulfonamide (190 mg, 0.446 mmol), Pd(dppf)Cl₂ (27 mg, 0.037 mmol) and cesium carbonate (364 mg, 1.116 mmol) were added to dioxane/water mixed solvent (5 mL, v/v=10:1). After the addition was completed, in the presense of protective nitrogen, the reaction mixture was heated to 100° C., stirred and reacted for 3 h. Upon completion of the reaction, the reaction was quenched by adding water (30 mL). The reaction mixture was extracted with ethyl acetate (50 mL×2 times). The organic phases were combined, washed with saturated brine (30 mL), dried over anhydrous sodium sulfate and filtered. The filtrate was concentrated under reduced pressure. The crude product obtained was purified by silica gel column chromatography (Eluent: methanol/dichloromethane=1/100 to 1/30). Tert-butyl 4-(6-(5-((2,4-difluorophenyl)sulfonamido)-6-methoxypyridin-3-yl)pyrido[3,2-d]pyrimidin-4-yl)piperazine-1-carboxylate was obtained, yield: 79.0%; ESI-MS(m/z): 614.2 [M+H]⁺.

### Step c): Preparation of 2,4-difluoro-N-(2-methoxy-5-(4-(piperazin-1-yl)pyrido[3,2-d]pyrimidin-6-yl)pyridin-3-yl)benzenesulfonamide trifluoroacetate

Tert-butyl 4-(6-(5-((2,4-difluorophenyl)sulfonamido)-6-methoxypyridin-3-yl)pyrido[3,2-d]pyrimidin-4-yl)piperazine-1-carboxylate (130 mg, 0.212 mmol) was dissolved in dichloromethane (4 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. The crude product was slurried with methyl tert-butyl ether (5 mL) to precipitate solid and filtered. 2,4-difluoro-N-(2-methoxy-5-(4-(piperazin-1-yl)pyrido[3,2-d]pyrimidin-6-ylpyridin-3-yl)benzenesulfonamide trifluoroacetate was obtained, yield: 97.1%; ESI-MS(m/z): 514.2 [M+H]⁺.

### Step d): Preparation of (E)-2,4-difluoro-N-(2-methoxy-5-(4-(4-(4-oxopent-2-enoyl)piperazin-1-yl)pyrido[3,2-d]pyrimidin-6-yl)pyridin-3-yl)benzenesulfonamide

Referring to the preparation method of Example 131, Step d. (E)-2,4-difluoro-N-(2-methoxy-5-(4-(4-(4-oxopent-2-enoyl)piperazin-1-yl)pyrido[3,2-d]pyrimidin-6-yl)pyridin-3-yl)benzenesulfonamide was obtained, yield: 21.9%; ¹H NMR (400 MHz, DMSO-d₆) δ 10.36 (brs, 1H), 8.79 (d, J=2.4 Hz, 1H), 8.58 (s, 1H), 8.45-8.38 (m, 2H), 8.22 (d, J=8.8 Hz, 1H), 7.81-7.72 (m, 1H), 7.60-7.51 (m, 1H), 7.47 (d, J=15.6 Hz, 1H), 7.24-7.16 (m, 1H), 6.73 (d, J=15.6 Hz, 1H), 4.75-4.30 (m, 4H), 4.00-3.80 (m, 4H), 3.75 (s, 3H), 2.36 (s, 3H); ESI-MS(m/z): 610.0 [M+H]⁺.

### Example 133

### Preparation of (E)-2-fluoro-N-(2-methoxy-5-(4-(4-(4-oxopent-2-enoyl)piperazin-1-yl)pyrido[3,2-d]pyrimidin-6-yl)pyridin-3-yl)benzenesulfonamide

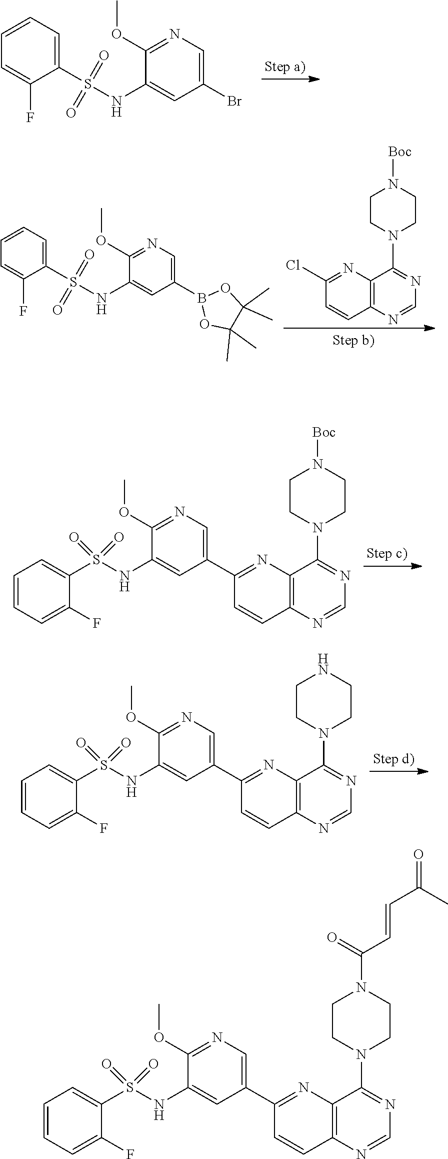

### Step a): Preparation of 2-fluoro-N-(2-methoxy-5-(4,4,5,5-tetramethyl-1,3,2-dioxaborolan-2-yl)pyridin-3-yl)benzenesulfonamide

N-(5-bromo-2-methoxypyridin-3-yl)-2-fluorobenzenesulfonamide (2.16 g, 5.980 mmol), pinacol diborate (2.28 g, 8.970 mmol), Pd(dppf)Cl₂ (438 mg, 0.598 mmol) and potassium acetate (1.76 g, 17.940 mmol) were added to 1,4-dioxane (20 mL). In the presense of protective nitrogen, the reaction mixture was kept at 90° C. and reacted for 3 h. Upon completion of the reaction, the reaction was quenched by adding water (50 mL). The reaction mixture was extracted with ethyl acetate (150 mL×2). The organic phases were combined, washed with saturated brine (50 mL), dried over anhydrous sodium sulfate and filtered. The filtrate was concentrated under reduced pressure. The crude product obtained was purified by silica gel column chromatography (Eluent: methanol/dichloromethane=1/100 to 1/50), 2-Fluoro-N-(2-methoxy-5-(4,4,5,5-tetramethyl-1,3,2-dioxaborolan-2-yl)pyridin-3-yl)benzenesulfonamide was obtained, yield: 97.2%; ESI-MS(m/z): 409.2 [M+H]⁺.

### Step b): Preparation of tert-butyl 4-(6-(54 (2-fluorophenyl)sulfonamido)-6-methoxypyridin-3-yl)pyrido[3,2-d]pyrimidin-4-yl)piperazine-1-carboxylate

2-Fluoro-N-(2-methoxy-5-(4,4,5,5-tetramethyl-1,3,2-dioxaborolan-2-yl)pyridin-3-yl)benzenesulfonamide (152 mg, 0.372 mmol), tert-butyl 4-(6-chloropyrido[3,2-d]pyrimidin-4-yl)piperazine-1-carboxylate (130 mg, 0.372 mmol), Pd(dppf)Cl₂ (27 mg, 0.037 mmol) and cesium carbonate (364 mg, 1.116 mmol) were added to dioxane/water mixed solvent (5 mL, v/v=10:1). After the addition was completed, in the presense of protective nitrogen, the reaction mixture was heated to 100° C., stirred and reacted for 3 h. Upon completion of the reaction, the reaction was quenched by adding water (30 mL). The reaction mixture was extracted with ethyl acetate (50 mL×2). The organic phases were combined, washed with saturated brine (30 mL), dried over anhydrous sodium sulfate and filtered. The filtrate was concentrated under reduced pressure. The crude product obtained was purified by silica gel column chromatography (Eluent: methanol/dichloromethane=1/100 to 1/30). Tert-butyl 4-(6-(5-((2-fluorophenyl)sulfonamido)-6-methoxypyridin-3-yl)pyrido[3,2-d]pyrimidin-4-yl)piperazine-1-carboxylate was obtained, yield: 45.4%; ESI-MS(m/z): 596.2 [M+H]⁺.

### Step c): Preparation of 2-fluoro-N-(2-methoxy-5-(4-(piperazin-1-yl)pyrido[3,2-d]pyrimidin-6-yl)pyridin-3-yl)benzenesulfonamide trifluoroacetate

Tert-butyl 4-(6-(5-((2-fluorophenyl)sulfonamido)-6-methoxypyridin-3-yl)pyrido[3,2-d]pyrimidin-4-yl)piperazine-1-carboxylate (100 mg, 0.168 mmol) was dissolved in dichloromethane (4 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. The crude product was slurried with methyl tert-butyl ether (5 mL) to precipitate solid and filtered. 2-Fluoro-N-(2-methoxy-5-(4-(piperazin-1-yl)pyrido[3,2-d]pyrimidin-6-yl)pyridin-3-yl)benzenesulfonamide trifluoroacetate was obtained, yield: 98.6%; ESI-MS(m/z): 496.2 [M+H]⁺.

### Step d): Preparation of (E)-2-fluoro-N-(2-methoxy-5-(4-(4-(4-oxopent-2-enoyl)piperazin-1-yl)pyrido[3,2-d]pyrimidin-6-yl)pyridin-3-yl)benzenesulfonamide

Referring to the preparation method of Example 131, Step d, (E)-2-fluoro-N-(2-methoxy-5-(4-(4-(4-oxopent-2-enoyl)piperazin-1-yl)pyrido[3,2-d]pyrimidin-6-yl)pyridin-3-yl)benzenesulfonamide was obtained, yield: 29.7%; ¹H NMR (400 MHz, DMSO-d₆) δ 10.28 (brs, 1H), 8.78 (d, J=2.0 Hz, 1H), 8.58 (s, 1H), 8.45-8.35 (m, 2H), 8.22 (d, J=8.8 Hz, 1H), 7.74-7.64 (m, 2H), 7.51-7.40 (m, 2H), 7.34-7.26 (m, 1H), 6.73 (d, J=16.0 Hz, 1H), 4.73-4.30 (m, 4H), 3.99-3.88 (m, 2H), 3.88-3.78 (m, 2H), 3.73 (s, 3H), 2.37 (s, 3H); ESI-MS(m/z): 592.0 [M+H]⁺.

### Example 134

### Preparation of (E)-4-fluoro-N-(2-methoxy-5-(4-(4-(4-oxopent-2-enoyl)piperazin-1-yl)pyrido[3,2-d]pyrimidin-6-yl)pyridin-3-yl)benzenesulfonamide

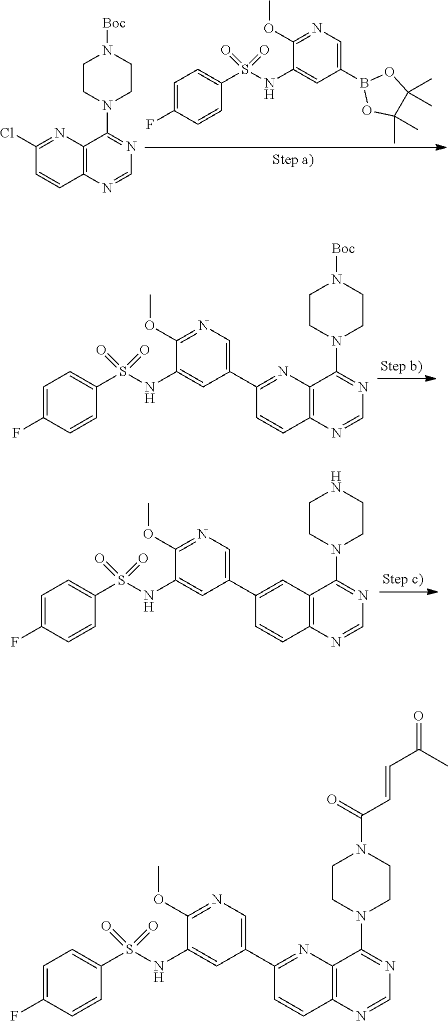

Referring to the preparation method of Example 3, After purified by Prep-HPLC (Method 2), (E)-4-fluoro-N-(2-methoxy-5-(4-(4-(4-oxopent-2-enoyl)piperazin-1-yl)pyrido[3,2-d]pyrimidin-6-yl)pyridin-3-yl)benzenesulfonamide was obtained, yield: 15.5%; ¹H NMR (400 MHz, DMSO-d₆) δ 10.07 (brs, 1H), 8.76 (d, J=2.4 Hz, 1H), 8.58 (s, 1H), 8.46-8.38 (m, 2H), 8.22 (d, J=8.8 Hz, 1H), 7.86-7.76 (m, 2H), 7.47 (d, J=16.0 Hz, 1H), 7.42-7.33 (m, 2H), 6.73 (d, J=15.6 Hz, 1H), 4.54 (s, 4H), 4.00-3.89 (m, 2H), 3.89-3.79 (m, 2H), 3.75 (s, 3H), 2.36 (s, 3H); ESI-MS(m/z): 592.0 [M+H]⁺.

### Example 135

### Preparation of (E)-2-chloro-4-fluoro-N-(2-methoxy-5-(4-(4-(4-oxopent-2-enoyl)piperazin-1-yl)pyrido[3,2-d]pyrimidin-6-yl)pyridin-3-yl)benzenesulfonamide

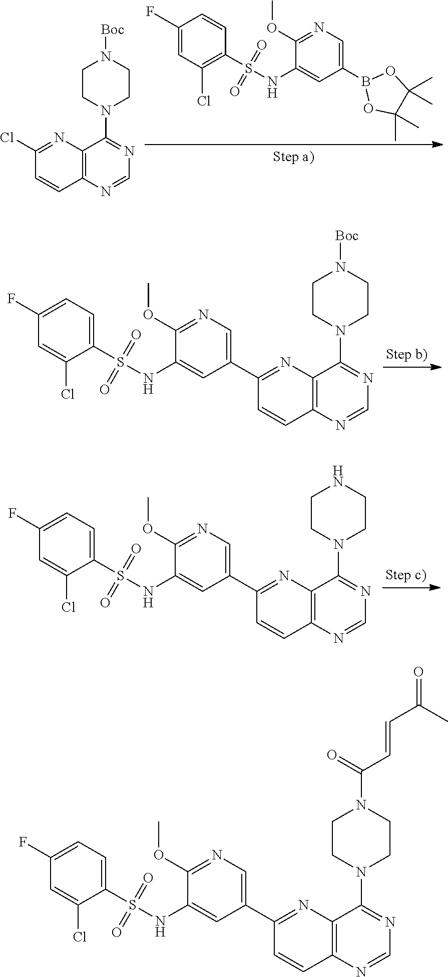 [SMILES: CC(C)(C)OC(=O)N1CCN(c2ncnc3ccc(Cl)nc23)CC1 ; COc1ncc(B2OC(C)(C)C(C)(C)O2)cc1NS(=O)(=O)c1ccc(F)cc1Cl ; COc1ncc(-c2ccc3ncnc(N4CCN(C(=O)OC(C)(C)C)CC4)c3n2)cc1NS(=O)(=O)c1ccc(F)cc1Cl ; COc1ncc(-c2ccc3ncnc(N4CCNCC4)c3n2)cc1NS(=O)(=O)c1ccc(F)cc1Cl ; COc1ncc(-c2ccc3ncnc(N4CCN(C(=O)/C=C/C(C)=O)CC4)c3n2)cc1NS(=O)(=O)c1ccc(F)cc1Cl]

### Step a): Preparation of tert-butyl 4-(6-(5-((2-chloro-4-fluorophenyl)sulfonamido)-6-methoxypyridin-3-yl)pyrido[3,2-d]pyrimidin-4-yl)piperazine-1-carboxylate

Tert-butyl 4-(6-chloropyrido[3,2-d]pyrimidin-4-yl)piperazine-1-carboxylate (100 mg, 0.286 mmol), 2-chloro-4-fluoro-N-(2-methoxy-5-(4,4,5,5-tetramethyl-1,3,2-dioxaborolan-2-yl)pyridin-3-yl)benzenesulfonamide (152 mg, 0.343 mmol), Pd(dppf)Cl₂ (21 mg, 0.029 mmol) and cesium carbonate (280 mg, 0.858 mmol) were added to dioxane/water mixed solvent (10 mL, v/v=10:1). After the addition was completed, in the presense of protective nitrogen, the reaction mixture was heated to 100° C., stirred and reacted for 3 h. Upon completion of the reaction, the reaction was quenched by adding water (30 mL). The reaction mixture was extracted with ethyl acetate (50 mL 2). The organic phases were combined, washed with saturated brine (30 mL), dried over anhydrous sodium sulfate and filtered. The filtrate was concentrated under reduced pressure. The crude product obtained was purified by silica gel column chromatography (Eluent: methanol/dichloromethane=1/100 to 1/30). Tert-butyl 4-(6-(5-((2-chloro-4-fluorophenyl)sulfonamido)-6-methoxypyridin-3-yl)pyrido[3,2-d]pyrimidin-4-yl)piperazine-1-carboxylate was obtained, yield: 87.6%; ESI-MS(m/z): 630.2 [M+H]⁺.

### Step b): Preparation of 2-chloro-4-fluoro-N-(2-methoxy-5-(4-(piperazin-1-yl)pyrido[3,2-d]pyrimidin-6-yl)pyridin-3-yl)benzenesulfonamide trifluoroacetate

Tert-butyl 4-(6-(5-((2-chloro-4-fluorophenyl)sulfonamido)-6-methoxypyridin-3-yl)pyrido[3,2-d]pyrimidin-4-yl)piperazine-1-carboxylate (160 mg, 0.254 mmol) was dissolved in dichloromethane (4 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. The crude product was slurried with methyl tert-butyl ether (5 mL) to precipitate solid and filtered. 2-Chloro-4-fluoro-N-(2-methoxy-5-(4-(piperazin-1-yl)pyrido[3,2-d]pyrimidin-6-yl)pyridin-3-yl)benzenesulfonamide trifluoroacetate was obtained trifluoroacetate, yield: 74.5%; ESI-MS(m/z): 530.2 [M+H]⁺.

### Step c): Preparation of (E)-2-chloro-4-fluoro-N-(2-methoxy-5-(4-(4-(4-oxopent-2-enoyl)piperazin-1-yl)pyrido[3,2-d]pyrimidin-6-yl)pyridin-3-yl)benzenesulfonamide

Referring to the preparation method of Example 131 Step d, (E)-2-chloro-4-fluoro-N-(2-methoxy-5-(4-(4-(4-oxopent-2-enoyl)piperazin-1-yl)pyrido[3,2-d]pyrimidin-6-yl)pyridin-3-yl)benzenesulfonamide was obtained, yield: 18.6%; ¹H NMR (400 MHz, DMSO-d₆) δ 10.23 (brs, 1H), 8.77 (d, J=2.4 Hz, 1H), 8.57 (s, 1H), 8.44-8.34 (m, 2H), 8.21 (d, J=8.8 Hz, 1H), 7.95-7.91 (m, 1H), 7.73-7.71 (m, 1H), 7.47 (d, J=16.0 Hz, 1H), 7.38-7.28 (m, 1H), 6.74 (d, J=15.6 Hz, 1H), 4.51 (s, 4H), 3.99-3.89 (m, 2H), 3.88-3.80 (m, 2H), 3.77 (s, 3H), 2.37 (s, 3H); ESI-MS(m/z): 626.0 [M+H]⁺.

### Example 136

### Preparation of (E)-2,6-difluoro-N-(2-methoxy-5-(4-(2-(4-oxopent-2-enoyl)-2,7-diazaspiro[3.5]nonan-7-yl)pyrido[3,2-d]pyrimidin-6-yl)pyridin-3-yl)benzenesulfonamide

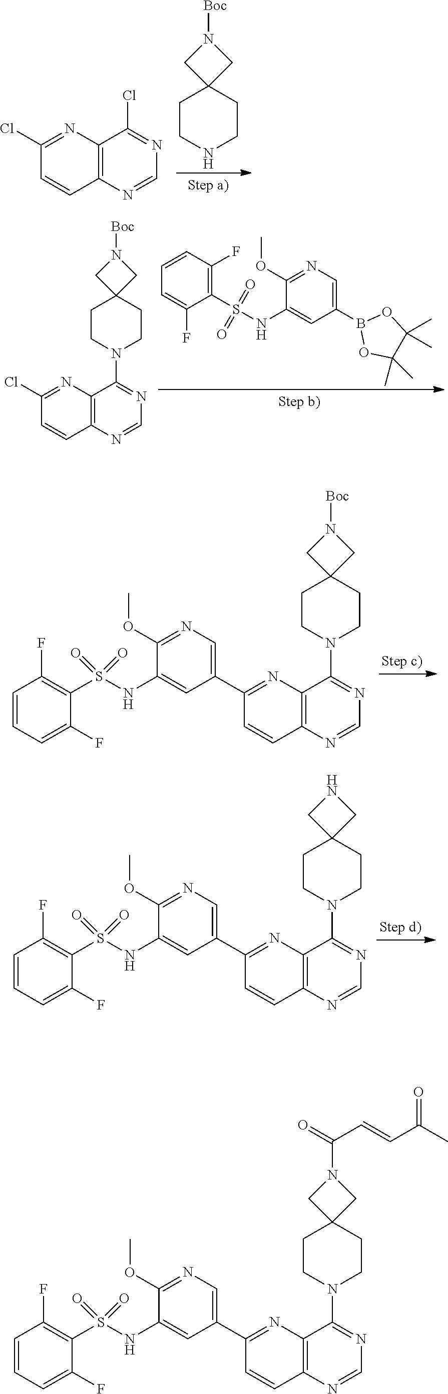

### Step a): Preparation of tert-butyl 7-(6-chloropyrido[3,2-d]pyrimidin-4-yl)-2,7-diazaspiro[3.5]nonane-2-carboxylate

4,6-Dichloropyrido[3,2-d]pyrimidine (150 mg, 0.750 mmol), tert-butyl 2,7-diazaspiro[3.5]nonane-2-carboxylate (186 mg, 0.825 mmol) was dissolved in N,N-dimethylformamide (5 mL), with N,N-diisopropylethylamine (290 mg, 2.250 mmol) added. After the addition was completed, the reaction mixture was heated to 40° C. and stirred for 4 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated under reduced pressure. The residue was purified by silica gel column chromatography (Eluent: dichloromethane/methanol=100/1 to 10/1). Tert-butyl 7-(6-chloropyrido[3,2-d]pyrimidin-4-yl)-2,7-diazaspiro[3.5]nonane-2-carboxylate was obtained, yield: 85.5%; ESI-MS(m/z): 390.5 [M+H]⁺.

### Step b): Preparation of tert-butyl 7-(6-(5-((2,6-difluorophenyl)sulfonamido)-6-methoxypyridin-3-yl)pyrido[3,2-d]pyrimidin-4-yl)-2,7-diazaspiro[3.5]nonane-2-carboxylate

Tert-butyl 7-(6-chloropyrido[3,2-d]pyrimidin-4-yl)-2,7-diazaspiro[3.5]nonane-2-carboxylate (250 mg, 0.641 mmol), 2,6-difluoro-N-(2-methoxy-5-(4,4,5,5-tetramethyl-1,3,2-dioxaborolan-2-yl)pyridin-3-yl)benzenesulfonamide (328 mg, 0.769 mmol), Pd(dppf)Clₕ(47 mg, 0.064 mmol) and cesium carbonate (627 mg, 1.923 mmol) were added to dioxane/water mixed solvent (10 mL, v/v=4:1). After the addition was completed, in the presense of protective nitrogen, the reaction mixture was heated to reflux and stirred for 2 h. Upon completion of the reaction, the reaction was quenched by adding water (50 mL). The reaction mixture was extracted with ethyl acetate (200 mL×2). The organic phases were combined, washed with saturated brine (100 mL), dried over anhydrous sodium sulfate and filtered. The filtrate was concentrated under reduced pressure. The crude product obtained was purified by silica gel column chromatography (Eluent: dichloromethane/methanol=100/1 to 10/1). Tert-butyl 7-(6-(5-((2,6-difluorophenyl)sulfonamido)-6-methoxypyridin-3-yl)pyrido[3,2-d]pyrimidin-4-yl)-2,7-diazaspiro[3.5]nonane-2-carboxylate was obtained, yield: 95.5%; ESI-MS(m/z): 654.2 [M+H]⁺.

### Step c): Preparation of N-(5-(4-(2,7-diazaspiro[3.5]nonan-7-yl)pyrido[3,2-d]pyrimidin-6-yl)-2-methoxypyridin-3-yl)-2,6-difluorobenzenesulfonamide trifluoroacetate

Tert-butyl 7-(6-(5-((2,6-difluorophenyl)sulfonamido)-6-methoxypyridin-3-yl)pyrido[3,2-d]pyrimidin-4-yl)-2,7-diazaspiro[3.5]nonane-2-carboxylate (400 mg, 0.612 mmol) was dissolved in dichloromethane (6 mL). Under the ice bath condition, trifluoroacetic acid (1.5 mL) was slowly added dropwise. After the addition was completed, the reaction mixture was stirred at room temperature for 1 h. Upon completion of the reaction, the reaction mixture was concentrated under reduced pressure. Methyl tert-butyl ether was added to the crude product. The mixture was stirred to precipitate solid and filtered. N-(5-(4-(2,7-diazaspiro[3.5]nonan-7-yl)pyrido[3,2-d]pyrimidin-6-yl)-2-methoxypyridin-3-yl)-2,6-difluorobenzenesulfonamide trifluoroacetate was obtained; ESI-MS(m/z): 545.5 [M+H]⁺.

### Step d): Preparation of (E)-2,6-difluoro-N-(2-methoxy-5-(4-(2-(4-oxopent-2-enoyl)-2,7-diazaspiro[3.5]nonan-7-yl)pyrido[3,2-d]pyrimidin-6-yl)pyridin-3-yl)benzenesulfonamide

N-(5-(4-(2,7-diazaspiro[3.5]nonan-7-yl)pyrido[3,2-d]pyrimidin-6-yl)-2-methoxypyridin-3-yl)-2,6-difluorobenzenesulfonamide trifluoroacetate (150 mg, 0.230 mmol) and tetrahydrofuran (6 mL) were added to the reaction flask. The reaction system was cooled to −78° C., with N,N-diisopropylethylamine (150 mg, 1.150 mmol), (E)-4-oxopent-2-enoic acid (29 mg, 0.253 mmol) and 50% T₃P ethyl acetate solution (293 mg, 0.460 mmol) added successively. After the addition was completed, the reaction mixture was kept at −78° C. and stirred for 1 h. Upon completion of the reaction, at −78° C., the reaction was quenched using saturated aqueous ammonium chloride solution (50 mL). The reaction mixture was extracted by adding dichloromethane (100 mL×2). The organic phases were combined, washed with saturated brine (100 mL), dried over anhydrous sodium sulfate and filtered. The filtrate was concentrated under reduced pressure. The crude product obtained was purified by Prep-HPLC (Method 2). (E)-2,6-difluoro-N-(2-methoxy-5-(4-(2-(4-oxopent-2-enoyl)-2,7-diazaspiro[3.5]nonan-7-yl)pyrido[3,2-d]pyrimidin-6-yl)pyridin-3-yl)benzenesulfonamide was obtained, yield: 16.8%; ¹H NMR (400 MHz, DMSO-d₅) S 10.51 (s, 1H), 8.82-8.71 (m, 1H), 8.51 (s, 1H), 8.43-8.34 (m, 2H), 8.17 (d, J=8.8 Hz, 1H), 7.74-7.63 (m, 1H), 7.25 (t, J=9.2 Hz, 2H), 6.95 (d, J=15.6 Hz, 1H), 6.75 (d, J=15.6 Hz, 1H), 4.49-4.32 (m, 4H), 4.15 (s, 2H), 3.79 (s, 2H), 3.71 (s, 3H), 2.35 (s, 3H), 1.97-1.87 (m, 4H); ESI-MS(m/z): 650.0 [M+H]⁺.

### Example 137

### Preparation of 2,6-difluoro-N-(2-methoxy-5-(4-(8-((E)-4-oxopent-2-enoyl)-3,8-diazabicyclo[3.2.1]octan-3-yl)pyrido[3,2-d]pyrimidin-6-yl)pyridin-3-yl)benzenesulfonamide

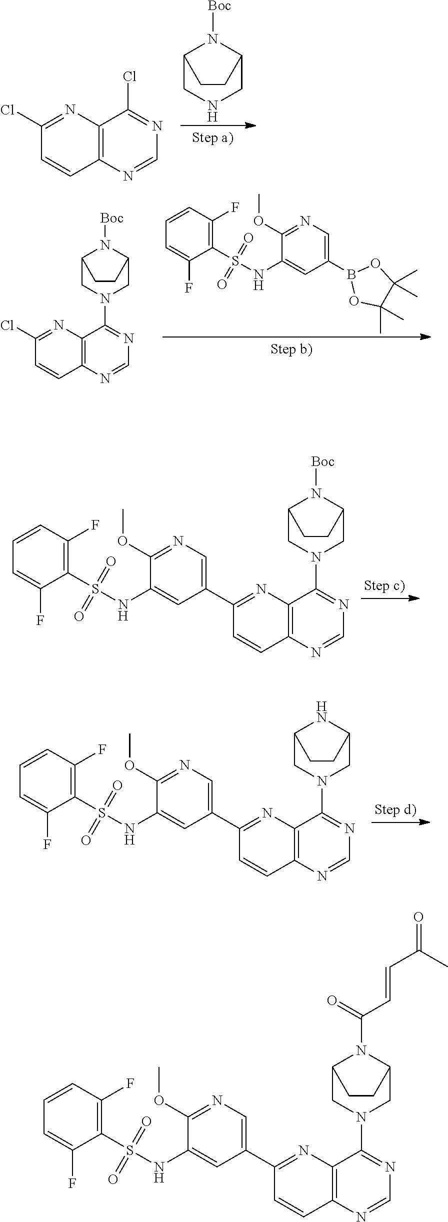

### Step a): Preparation of tert-butyl 3-(6-chloropyrido[3,2-d]pyrimidin-4-yl)-3,8-diazabicyclo[3.2.1]octane-8-carboxylate

4,6-Dichloropyrido[3,2-d]pyrimidine (150 mg, 0.750 mmol), tert-butyl 3,8-diazabicyclo[3.2.1]octane-8-carboxylate (175 mg, 0.825 mmol) were dissolved in N,N-dimethylformamide (5 mL), with N,N-diisopropylethylamine (290 mg, 2.250 mmol) added. After the addition was completed, the reaction mixture was heated to 40° C. and stirred for 4 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated under reduced pressure. The residue was purified by silica gel column chromatography (Eluent: dichloromethane/methanol=100/1 to 10/1). Tert-butyl 3-(6-chloropyrido[3,2-d]pyrimidin-4-yl)-3,8-diazabicyclo[3.2.1]octane-8-carboxylate was obtained, yield: 92.2%; ESI-MS(m/z): 376.2 [M+H]⁺.

### Step b): Preparation of tert-butyl 3-(6-(5-((2,6-difluorophenyl)sulfonamido)-6-methoxypyridin-3-yl)pyrido[3,2-d]pyrimidin-4-yl)-3,8-diazabicyclo[3.2.1]octane-8-carboxylate

Tert-butyl 3-(6-chloropyrido[3,2-d]pyrimidin-4-yl)-3,8-diazabicyclo[3.2.1]octane-8-carboxylate (260 mg, 0.691 mmol), 2,6-difluoro-N-(2-methoxy-5-(4,4,5,5-tetramethyl-1,3,2-dioxaborolan-2-yl)pyridin-3-yl)benzenesulfonamide (353 mg, 0.830 mmol), Pd(dppf)Cl₂ (51 mg, 0.069 mmol) and cesium carbonate (676 mg, 2.073 mmol) were added to dioxane/water mixed solvent (10 mL, v/v=4:1). After the addition was completed, in the presense of protective nitrogen, the reaction mixture was heated to 110° C. and stirred for 2 h. Upon completion of the reaction, the reaction was quenched by adding water (50 mL). The reaction mixture was extracted with ethyl acetate (200 mL 2). The organic phases were combined, washed with saturated brine (100 mL), dried over anhydrous sodium sulfate and filtered. The filtrate was concentrated under reduced pressure. The crude product obtained was purified by silica gel column chromatography (Eluent: dichloromethane/methanol=100/1 to 10/1).

Tert-butyl 3-(6-(5-((2,6-difluorophenyl)sulfonamido)-6-methoxypyridin-3-yl)pyrido[3,2-d]pyrimidin-4-yl)-3,8-diazabicyclo[3.2.1]octane-8-carboxylate was obtained, yield: 90.3%; ESI-MS(m/z): 640.2 [M+H]⁺.

### Step c): Preparation of N-(5-(4-(3,8-diazabicyclo[3.2.1]octan-3-yl)pyrido[3,2-d]pyrimidin-6-yl)-2-methoxypyridin-3-yl)-2,6-difluorobenzenesulfonamide trifluoroacetate

Tert-butyl 3-(6-(5-((2,6-difluorophenyl)sulfonamido)-6-methoxypyridin-3-yl)pyrido[3,2-d]pyrimidin-4-yl)-3,8-diazabicyclo[3.2.1]octane-8-carboxylate (400 mg, 0.612 mmol) was dissolved in dichloromethane (6 mL). Under the ice bath condition, trifluoroacetic acid (1.5 mL) was slowly added dropwise. After the addition was completed, the reaction mixture was stirred at room temperature for 1 h. Upon completion of the reaction, the reaction mixture was concentrated under reduced pressure. Methyl tert-butyl ether was added to the crude product. The mixture was stirred to precipitate solid and filtered. N-(5-(4-(3,8-diazabicyclo[3.2.1]octan-3-yl)pyrido[3,2-d]pyrimidin-6-yl)-2-methoxypyridin-3-yl)-2,6-difluorobenzenesulfonamide trifluoroacetate was obtained; ESI-MS(m/z): 540.5 [M+H]⁺.

### Step d): Preparation of 2,6-difluoro-N-(2-methoxy-5-(4-(8-((E)-4-oxopent-2-enoyl)-3,8-diazabicyclo[3.2.1]octan-3-yl)pyrido[3,2-d]pyrimidin-6-yl)pyridin-3-yl)benzenesulfonamide

N-(5-(4-(3,8-diazabicyclo[3.2.1]octan-3-yl)pyrido[3,2-d]pyrimidin-6-yl)-2-methoxypyridin-3-yl)-2,6-difluorobenzenesulfonamide trifluoroacetate (150 mg, 0.235 mmol) and tetrahydrofuran (6 mL) were added to the reaction flask. The reaction system was cooled to −78° C., with N,N-diisopropylethylamine (152 mg, 1.175 mmol), (E)-4-oxopent-2-enoic acid (29 mg, 0.259 mmol) and 50% T₃P ethyl acetate solution (299 mg, 0.470 mmol, 50% wt) added successively. After the addition was completed, the reaction mixture was kept at −78° C. and stirred for 1 h. Upon completion of the reaction, at −78° C., the reaction was quenched using saturated aqueous ammonium chloride solution (50 mL). The reaction mixture was extracted by adding dichloromethane (100 mL×2). The organic phases were combined, washed with saturated brine (100 mL), dried over anhydrous sodium sulfate and filtered. The filtrate was concentrated under reduced pressure. The crude product obtained was purified by Prep-HPLC (Method 2), 2,6-Difluoro-N-(2-methoxy-5-(4-(8-((E)-4-oxopent-2-enoyl)-3,8-diazabicyclo[3.2.1]octan-3-yl)pyrido[3,2-d]pyrimidin-6-yl)pyridin-3-yl)benzenesulfonamide was obtained, yield: 15.8%; ¹H NMR (400 MHz, DMSO-d₆) δ 10.62 (s, 1H), 8.77 (s, 1H), 8.57 (s, 1H), 8.49-8.35 (m, 2H), 8.22 (d, J=8.8 Hz, 1H), 7.75-7.62 (m, 1H), 7.41 (d, J=15.6 Hz, 1H), 7.25 (t, J=9.2 Hz, 2H), 6.81 (d, J=15.6 Hz, 1H), 5.74-5.23 (m, 2H), 4.97-4.75 (m, 2H), 3.74 (s, 3H), 3.59-3.34 (m, 2H), 2.38 (s, 3H), 2.04-1.79 (m, 4H); ESI-MS(m/z): 636.0 [M+H]⁺.

### Example 138

### Preparation of (S,E)-2,6-difluoro-N-(2-methoxy-5-(4-(2-methyl-4-(4-oxopent-2-enoyl)piperazin-1-yl)pyrido[3,2-d]pyrimidin-6-yl)pyridin-3-yl)benzenesulfonamide

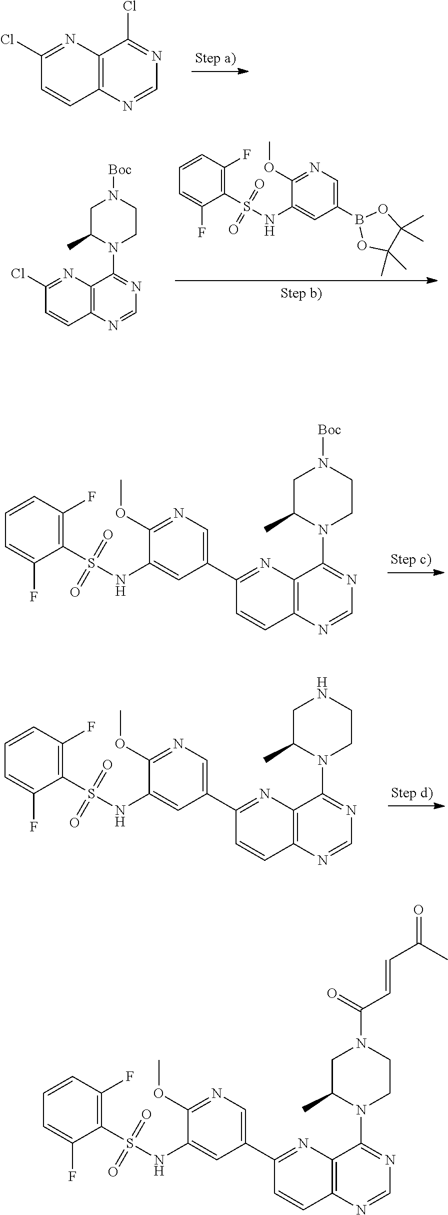

### Step a): Preparation of tert-butyl (S)-4-(6-chloropyrido[3,2-d]pyrimidin-4-yl)-3-methylpiperazine-1-carboxylate

4,6-Dichloropyrido[3,2-d]pyrimidine (150 mg, 0.750 mmol), tert-butyl (S)-3-methylpiperazine-1-carboxylate (165 mg, 0.825 mmol) were dissolved in N,N-dimethylformamide (5 mL), with N,N-diisopropylethylamine (290 mg, 2.250 mmol) added. After the addition was completed, the reaction mixture was heated to 40° C. and stirred for 4 h. Upon completion of the reaction, the reaction was quenched by adding water (50 mL). The reaction mixture was extracted with ethyl acetate (100 mL×2). The organic phases were combined, dried over anhydrous sodium sulfate and filtered. The filtrate was concentrated under reduced pressure. The residue was purified by silica gel column chromatography (Eluent: dichloromethane/methanol=100/1 to 10/1). Tert-butyl (S)-4-(6-chloropyrido[3,2-d]pyrimidin-4-yl)-3-methylpiperazine-1-carboxylate was obtained, yield: 95.2%; ESI-MS(m/z): 364.2 [M+H]⁺.

### Step b): Preparation of tert-butyl (S)-4-(6-(5-((2,6-difluorophenyl)sulfonamido)-6-methoxypyridin-3-yl)pyrido[3,2-d]pyrimidin-4-yl)-3-methylpiperazine-1-carboxylate

Tert-butyl (S)-4-(6-chloropyrido[3,2-d]pyrimidin-4-yl)-3-methylpiperazine-1-carboxylate (260 mg, 0.714 mmol), 2,6-difluoro-N-(2-methoxy-5-(4,4,5,5-tetramethyl-1,3,2-dioxaborolan-2-yl)pyridin-3-yl)benzenesulfonamide (365 mg, 0.857 mmol), Pd(dppf)Cl₂ (52 mg, 0.071 mmol) and cesium carbonate (698 mg, 2.142 mmol) were added to dioxane/water mixed solvent (10 mL, v/v=4:1). After the addition was completed, in the presense of protective nitrogen, the reaction mixture was heated to 110° C. and stirred for 2 h. Upon completion of the reaction, the reaction was quenched by adding water (50 mL). The reaction mixture was extracted with ethyl acetate (200 mL×2). The organic phases were combined, washed with saturated brine (100 mL), dried over anhydrous sodium sulfate and filtered. The filtrate was concentrated under reduced pressure. The crude product obtained was purified by silica gel column chromatography (Eluent: dichloromethane/methanol=100/1 to 10/1). Tert-butyl (S)-4-(6-(5-((2,6-difluorophenyl)sulfonamido)-6-methoxypyridin-3-ylpyrido[3,2-d]pyrimidin-4-yl)-3-methylpiperazine-1-carboxylate was obtained, yield: 89.1%; ESI-MS(m/z): 628.2 [M+H]⁺.

### Step c): Preparation of (S)-4-(6-(5-((2,6-difluorophenyl)sulfonamido)-6-methoxypyridin-3-yl)pyrido[3,2-d]pyrimidin-4-yl)-3-methylpiperazine-1-carboxylate trifluoroacetate

Tert-butyl (S)-4-(6-(54 (2,6-difluorophenyl)sulfonamido)-6-methoxypyridin-3-yl)pyrido[3,2-d]pyrimidin-4-yl)-3-methylpiperazine-1-carboxylate (400 mg, 0.637 mmol) was dissolved in dichloromethane (6 mL). Under the ice bath condition, trifluoroacetic acid (1.5 mL) was slowly added dropwise. After the addition was completed, the reaction mixture was stirred at room temperature for 1 h. Upon completion of the reaction, the reaction mixture was concentrated under reduced pressure. Methyl tert-butyl ether was added to the crude product. The mixture was stirred to precipitate solid and filtered. (S)-4-(6-(5-((2,6-difluorophenyl)sulfonamido)-6-methoxypyridin-3-ylpyrido[3,2-d]pyrimidin-4-yl)-3-methylpiperazine-1-carboxylate trifluoroacetate was obtained; ESI-MS(m/z): 528.5 [M+H]⁺.

### Step d): Preparation of (S,E)-2,6-difluoro-N-(2-methoxy-5-(4-(2-methyl-4-(4-oxopent-2-enoyl)piperazin-1-yl)pyrido[3,2-d]pyrimidin-6-yl)pyridin-3-yl)benzenesulfonamide

(S)-4-(6-(5-((2,6-difluorophenyl)sulfonamido)-6-methoxypyridin-3-yl)pyrido[3,2-d]pyrimidin-4-yl)-3-methylpiperazine-1-carboxylate trifluoroacetate (150 mg, 0.240 mmol) was dissolved in tetrahydrofuran (6 mL). The reaction system was cooled to −78° C. with N,N-diisopropylethylamine (156 mg, 1.200 mmol), (E)-4-oxopent-2-enoic acid (30 mg, 0.264 mmol) and 50% T₃P ethyl acetate solution (305 mg, 0.480 mmol) added successively. After the addition was completed, the reaction mixture was kept at −78° C. and stirred for 1 h. Upon completion of the reaction, at −78° C., the reaction was quenched using saturated aqueous ammonium chloride solution (50 mL). The reaction mixture was extracted by adding dichloromethane (100 mL×2). The organic phases were combined, washed with saturated brine (100 mL), dried over anhydrous sodium sulfate and filtered. The filtrate was concentrated under reduced pressure. The crude product obtained was purified by Prep-HPLC (Method 2). (S,E)-2,6-difluoro-N-(2-methoxy-5-(4-(2-methyl-4-(4-oxopent-2-enoyl)piperazin-1-yl)pyrido[3,2-d]pyrimidin-6-yl)pyridin-3-yl)benzenesulfonamide was obtained, yield: 15.8%; ¹H NMR (400 MHz, DMSO-d₆) δ 10.61 (s, 1H), 8.79 (s, 1H), 8.57 (s, 1H), 8.49-8.31 (m, 2H), 8.22 (d, J=8.8 Hz, 1H), 7.77-7.63 (m, 1H), 7.46 (dd, J₁=29.6 Hz, J₂=16.0 Hz, 1H), 7.25 (t, J=9.2 Hz, 2H), 6.75 (dd, J₁=15.6 Hz, J₂=3.6 Hz, 1H), 4.55-4.02 (m, 3H), 3.91-3.73 (m, 1H), 3.72 (s, 3H), 3.64-3.44 (m, 1H), 3.32-3.08 (m, 2H), 2.37 (s, 3H), 1.46-1.24 (m, 3H); ESI-MS(m/z): 624.0 [M+H]⁺.

### Example 139

### Preparation of (E)-2,6-difluoro-N-(2-methoxy-5-(4-(4-(4-oxopent-2-enoyl)piperazin-1-yl)pyrido[2,3-d]pyrimidin-6-yl)pyridin-3-yl)benzenesulfonamide

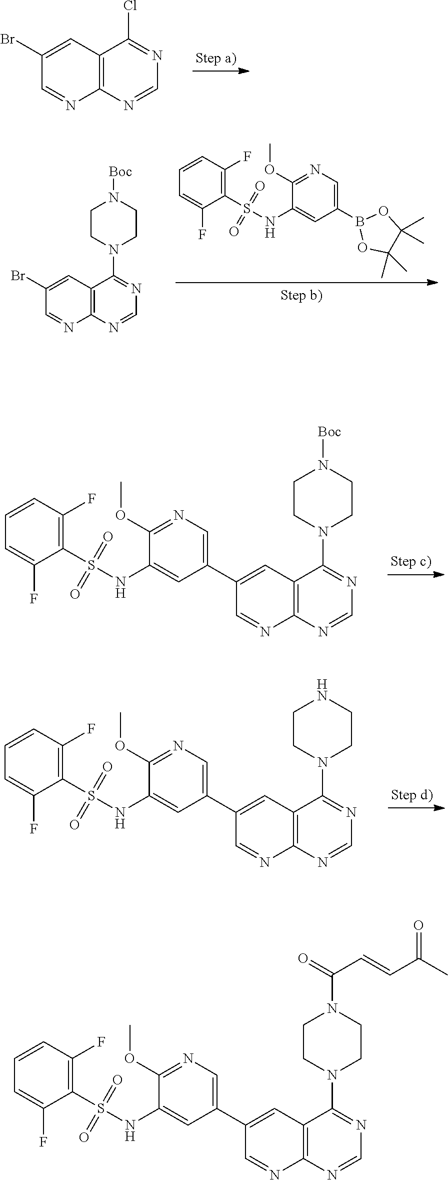

Referring to the preparation method of Example 131, (E)-2,6-difluoro-N-(2-methoxy-5-(4-(4-(4-oxopent-2-enoyl)piperazin-1-vi)pyrido[2,3-d]pyrimidin-6-yl)pyridin-3-yl)benzenesulfonamide was obtained, yield: 45.2%; ¹H NMR (400 MHz, DMSO) δ 10.60 (s, 1H), 9.27 (s, 1H), 8.74 (s, 11H), 8.52 (s, 1H), 8.43 (s, 1H), 8.04 (s, 1H), 7.68-7.64 (m, 1H), 7.44 (d, J=15.6 Hz, 1H), 7.23 (t, J=18.4 Hz, 2H), 6.74 (d, J=15.6 Hz, 1H), 4.02-3.98 (m, 4H), 3.90-3.88 (m, 2H), 3.81-3.78 (m, 2H), 3.66 (s, 3H), 2.37 (s, 3H); ESI-MS (m/z): 610.0 [M+H]⁺.

### Example 140

### Preparation of 2,4,6-trifluoro-N-(5-(4-(2-(2-fluoroacryloyl)-2,7-diazaspiro[3.5]nonan-7-yl) quinazoline-6-yl)-2-methoxypyridin-3-yl)benzenesulfonamide

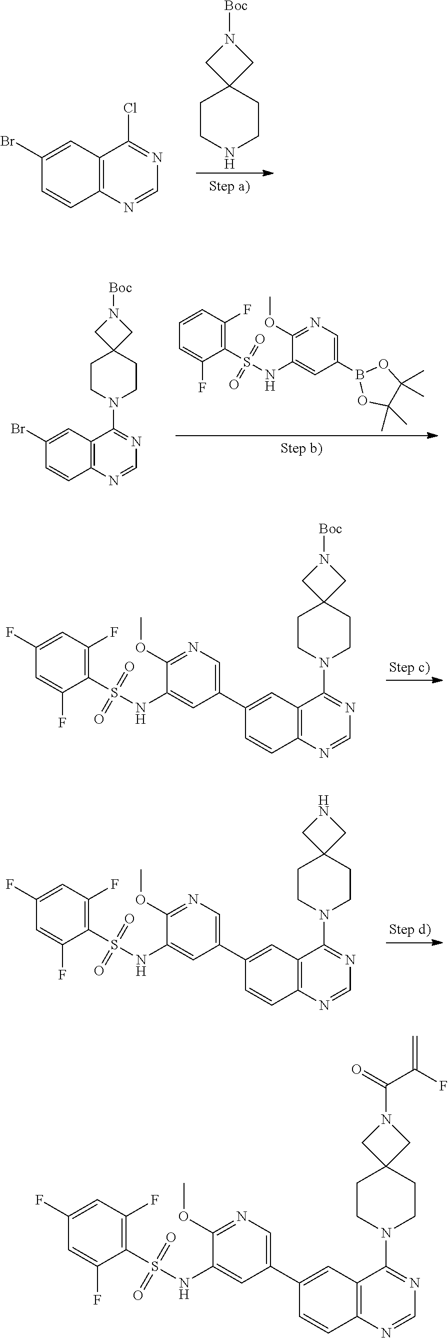

Step a): Preparation of tert-butyl 7-(6-bromoquinazolin-4-yl)-2,7-diazaspiro[3.5]nonane-2-carboxylate

6-Bromo-4-chloroquinazoline (1.5 g, 6.160 mmol) and tert-butyl 2,7-diazaspiro[3.5]nonane-2-carboxylate (1.4 g, 6.160) mmol) were dissolved in dimethyl sulfoxide (25 mL), with N,N-diisopropylethylamine (2.0 g, 15.349 mmol) added. After the addition was completed, the reaction mixture was heated to 50° C. and stirred for 4 h. Upon completion of the reaction, the reaction was quenched by adding iced water (80 mL). The reaction mixture was extracted with ethyl acetate (60 mL*2). The organic phases were combined, dried over anhydrous sodium sulfate and filtered. The filtrate was concentrated under reduced pressure. The obtained crude product was ultrasonically dispersed into 10 mL petroleum ether. The reaction mixture was stirred at room temperature for 0.5 h and filtered. The solid was collected and dried to obtain tert-butyl 7-(6-bromoquinazolin-4-yl)-2,7-diazaspiro[3.5]nonane-2-carboxylate, yield: 99.9%4; ESI-MS(m/z): 434.3 [M+H]⁺.

### Step b): Preparation of tert-butyl 74 (6-(6-methoxy-5-((2,4,6-trifluorophenyl)sulfonamido)pyridin-3-yl)quinazolin-4-yl)-2,7-diazaspiro[3.5]nonane-2-carboxylate

Tert-butyl 7-(6-bromoquinazolin-4-yl)-2,7-diazaspiro[3.5]nonane-2-carboxylate (200 mg, 0.464 mmol), 2,4,6-trifluoro-N-(2-methoxy-5-(4,4,5,5-tetramethyl-1,3,2-nitroxyborolan-2-yl)pyridin-3-yl)benzenesulfonamide (206 mg, 0.464 mmol), Pd(dppf)Cl₂ (34 mg, 0.046 mmol), cesium carbonate (450 mg, 1.382 mmol), dioxane (5 mL) and water (0.5 mL) were added to the reaction flask. After the addition was completed, in the presense of protective nitrogen, the reaction mixture was kept at 100° C. and stirred for 5 h. Upon completion of the reaction, the reaction mixture was cooled to room temperature, with the reaction quenched by adding saturated ammonium chloride (80 mL), extracted with ethyl acetate (80 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=5/1 to 1/2).

Tert-butyl 7-(6-(6-methoxy-5-((2,4,6-trifluorophenyl)sulfonamido)pyridin-3-yl)quinazolin-4-yl)-2,7-diazaspiro[3.5]nonane-2-carboxylate was obtained, yield: 98.6%. ESI-MS(m/z): 671.2 [M+H]⁺.

### Step c): Preparation of N-(5-(4-(2,7-diazaspiro[3.5]nonan-7-yl)quinazolin-6-yl)-2-methoxypyridin-3-yl)-2,4,6-trifluorobenzenesulfonamide trifluoroacetate

Tert-butyl 7-(6-(6-methoxy-5-((2,4,6-trifluorophenyl)sulfonamido)pyridin-3-yl)quinazolin-4-yl)-2,7-diazaspiro[3.5]nonane-2-carboxylate (338 mg, 0.504 mmol) was dissolved in dichloromethane (9 mL). Under the ice bath condition, TFA (3 mL) was slowly added dropwise. After the addition was completed, the reaction mixture was stirred at room temperature for 1 h. Upon completion of the reaction, the reaction mixture was concentrated under reduced pressure. The obtained product was dispersed into 10 mL methyl tert-butyl ether. The reaction mixture was stirred at room temperature for 0.5 h and filtered. The solid was collected and dried to obtain N-(5-(4-(2,7-diazaspiro[3.5]nonan-7-yl)quinazolin-6-yl)-2-methoxypyridin-3-yl)-2,4,6-trifluorobenzenesulfonamide trifluoroacetate, yield: 88.1%; ESI-MS(m/z): 571.1 [M+H]⁺.

### Step d): Preparation of 2,4,6-trifluoro-N-(5-(4-(2-(2-fluoroacryloyl)-2,7-diazaspiro[3.5]nonan-7-yl)quinazoline-6-yl)-2-methoxypyridin-3-yl)benzenesulfonamide

N-(5-(4-(2,7-diazaspiro[3.5]nonan-7-yl)quinazolin-6-yl)-2-methoxypyridin-3-yl)-2,4,6-trifluorobenzenesulfonamide trifluoroacetate (100 mg, 0.154 mmol), 2-fluoroacrylic acid (16 mg, 0.182 mmol), N,N-diisopropylethylamine (97 mg, 0.753 mmol) were dissolved in tetrahydrofuran (2 mL). At −78° C., T₃P (111 mg, 0.174 mmol, 50% wt/o) was slowly added to the system. After the addition was completed, the reaction mixture was kept at −78° C. and stirred for 0.5 h. Upon completion of the reaction, with 1 mL acetonitrile added, the reaction mixture was filtered and purified by Prep-HPLC (Method 2), 2,4,6-Trifluoro-N-(5-(4-(2-(2-fluoroacryloyl)-2,7-diazaspiro[3.5]nonan-7-yl)quinazoline-6-yl)-2-methoxypyridin-3-yl)benzenesulfonamide was obtained, yield: 26.7%; ¹H NMR (400 MHz, DMSO-d₆) δ 10.68 (s, 1H), 8.62 (s, 1H), 8.49-8.41 (m, 1H), 8.12-8.00 (m, 3H), 7.89 (d, J=8.4 Hz, 1H), 7.47-7.38 (m, 2H), 5.48 (dd, J₁=48.4 Hz, J₂=3.6 Hz, 1H), 5.29 (dd, J₁=16.4 Hz, J₁=3.6 Hz, 1H), 4.21-4.14 (m, 2H), 3.82-3.71 (m, 6H), 3.69 (s, 3H), 1.99-1.90 (m, 4H); ESI-MS(m/z): 643.0 [M+H]⁺.

Referring to the preparation method of Example 140 and using the corresponding raw materials, the compounds in the following examples were obtained.

| Num- | | | |
|---|---|---|---|
| ber | Name | Structure | ¹H NMR and MS |
| Ex- ample 141 | (E)-2,4-difluoro- N-(2-methoxy- 5-(4-(6-(4- oxopent-2- enoyl))-2,6- diazaspirocycle [3.3]heptan-2- yl)quinazolin-6- yl)pyridin-3-yl) benzene- sulfonamide | 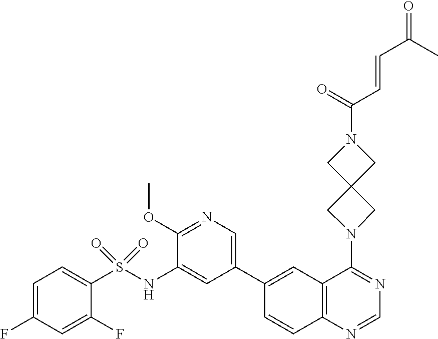 [SMILES: COc1ncc(-c2ccc3ncnc(N4CC5(CN(C(=O)/C=C/C(C)=O)C5)C4)c3c2)cc1NS(=O)(=O)c1ccc(F)cc1F] | ¹H NMR (400 MHz, DMSO-d₆) δ 10.22 (s, 1H), 8.48 (s, 1H), 8.43-8.41 (m, 1H), 8.05-8.01 (m, 2H), 7.98- 7.96 (m, 1H), 7.82-7.78 (m, 1H), 7.78-7.73 (m, 1H), 7.60-7.53 (m, 1H), 7.23-7.18 (m, 1H), 6.89 (d, J = 16.0 Hz, 1H), 6.75 (d, J = 16.0 Hz, 1H), 4.89-4.61 (m, 4H), 4.58 (s, 2H), 4.25 (s, 2H), 3.66 (s, 3H), 2.34 (s, 3H); ESI-MS(m/z): 621.0 [M + H]⁺. |
| Ex- ample 142 | (E)-2,4-difluoro- N-(2-methoxy- 5-(4-(6-(4- oxopent-2- enoyl)-3,6- diazabicyclo [3.1.1]heptan-3- yl)quinazolin-6- yl)pyridin-3-yl) benzene- sulfonamide | 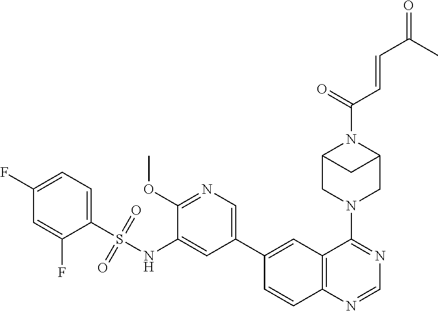 [SMILES: COc1ncc(-c2ccc3ncnc(N4CC5CC(C4)N5C(=O)/C=C/C(C)=O)c3c2)cc1NS(=O)(=O)c1ccc(F)cc1F] | ¹H NMR (400 MHz, DMSO-d₆) δ 10.28 (s, 1H), 8.55-8.50 (m, 2H), 8.41 (s, 1H), 8.01-7.96 (m, 2H), 7.85- 7.75(m, 2H), 7.58-7.55 (m, 1H), 7.23-7.19 (m, 1H), 7.05 (d, J = 16.0 Hz, 1H), 6.72 (d, J = 16.0 Hz, 1H), 5.03 (s, 1H), 4.61 (s, 1H), 4.51-4.45 (m, 3H), 4.37-4.33 (m, 1H), 3.68 (s, 3H), 2.75-2.73 (m, 1H), 2.34 (s, 3H), 1.72 (d, J = 12.0 Hz, 1H); ESI- MS(m/z): 621.0 [M + H]⁺. |
| Ex- ample 144 | 2,4-difluoro-N- (2-methoxy-5- (4-(3-((E)-4- oxopent-2- enoyl)-3,8- diazabicyclo [3.2.1]octan-8- yl)quinazolin-6- yl)pyridin-3-yl) benzene- sulfonamide | 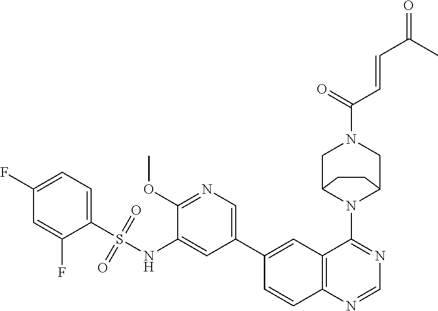 [SMILES: COc1ncc(-c2ccc3ncnc(N4C5CCC4CN(C(=O)/C=C/C(C)=O)C5)c3c2)cc1NS(=O)(=O)c1ccc(F)cc1F] | ¹H NMR (400 MHz, DMSO-d₆) δ 10.28 (s, 1H), 8.65 (s, 1H), 8.48 (s, 1H), 8.19-8.08 (m, 2H), 8.05 (s, 1H), 7.91 (d, J = 8 Hz, 1H), 7.78-7.75 (m, 1H), 7.56-7.53 (m, 1H), 7.43 (d, J = 16 Hz, 1H), 7.23-7.18 (m, 1H), 6.71 (d, J = 16 Hz, 1H), 4.98 (d, J = 12 Hz, 2H), 4.40 (d, J = 16 Hz, 1H), 4.10 (d, J = 19 Hz, 1H), 3.74 (d, J = 16 Hz, 1H), 3.69 (s, 3H), 3.28-3.25 (m, 1H), 2.35 (s, 3H), 1.99-1.87 (m, 2H), 1.80-1.75 (m, 1H), 1.66-1.61 (m, 1H); ESI-MS(m/z): 635.0 [M + H]⁺. |
| Ex- ample 145 | 2,6-difluoro-N- (2-methoxy-5- (4-(8-((E)-4- oxopent-2- enoyl)-3,8- diazabicyclo [3.2.1]octan-3- yl)quinazolin-6- yl)pyridin-3-yl) benzene- sulfonamide | 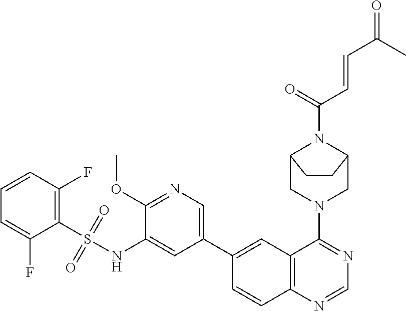 [SMILES: COc1ncc(-c2ccc3ncnc(N4CC5CCC(C4)N5C(=O)/C=C/C(C)=O)c3c2)cc1NS(=O)(=O)c1c(F)cccc1F] | ¹H NMR (400 MHz, DMSO-d₆) δ 10.58 (s, 1H), 8.60 (s, 1H), 8.42 (s, 1H), 8.09-8.02 (m, 2H), 7.98 (s, 1H), 7.88 (d, J = 8.0 Hz, 1H), 7.73-7.64 (m, 1H), 7.41 (d, J = 12.0 Hz, 1H), 7.26 (t, J = 8.0 Hz, 2H), 6.84 (d, J = 16.0 Hz, 1H), 4.80-4.72 (m, 2H), 4.56-4.44 (m, 2H), 3.64 (s, 3H), 3.63-3.55 (m, 2H), 2.39 (s, 3H), 1.97-1.76 (m, 4H); ESI-MS(m/z): 635.0 [M + H]⁺. |
| Ex- ample 146 | 2,4-difluoro-N- (5-(4-(8-(2- fluoroacryloyl)- 3,8- diazabicyclo [3.2.1]octan-3- yl)quinazolin-6- yl)-2- methoxypyridin- 3-yl) benzene- sulfonamide | 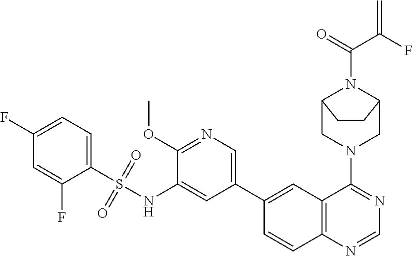 [SMILES: C=C(F)C(=O)N1C2CCC1CN(c1ncnc3ccc(-c4cnc(OC)c(NS(=O)(=O)c5ccc(F)cc5F)c4)cc13)C2] | ¹H NMR (400 MHz, DMSO-d₆) δ 10.34 (s, 1H), 8.60 (s, 1H), 8.45 (s, 1H), 8.09-8.04 (m, 2H), 8.00-7.98 (m, 1H), 7.88 (d, J = 8.0 Hz, 1H), 7.80-7.73 (m, 1H), 7.60-7.53 (m, 1H), 7.24-7.18 (m, 1H), 5.52-5.41 (m, 1H), 5.40-5.36 (m, 1H), 4.72- 4.60 (m, 2H), 4.51 (d, J = 16.0 Hz, 2H), 3.67 (s, 3H), 3.65-3.59 (m, 2H), 1.93-1.75 (m, 4H); ESI-MS(m/z): 611.0 [M + H]⁺. |
| Ex- ample 147 | N-(5-(4-((1- acryloyl- pyrrolidin- 3-yl)amino) quinazolin- 6-yl)-2- methoxypyridin- 3-yl)-2,4- difluoro- benzene- sulfonamide | 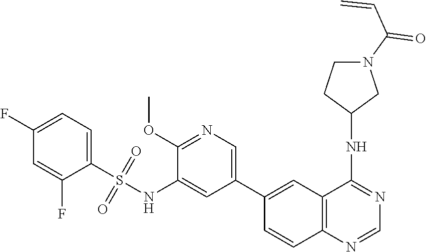 [SMILES: C=CC(=O)N1CCC(Nc2ncnc3ccc(-c4cnc(OC)c(NS(=O)(=O)c5ccc(F)cc5F)c4)cc23)C1] | ¹H NMR (400 MHz, DMSO-d₆) δ 8.55-8.52 (m, 2H), 8.31-8.26 (m, 1H), 8.17 (s, 1H),7.91 (d, J = 8.0 Hz, 1H), 7.84-7.76 (m, 3H), 7.40 (t, J = 8.0 Hz, 1H), 7.16-7.11 (m, 1H), 6.68-6.56 (m, 1H), 6.19-6.13 (m, 1H), 5.71-5.65 (m, 1H), 4.92-4.77 (m, 1H), 4.08-4.04 (m, 1H), 3.88- 3.50 (m, 7H), 2.39-2.22 (m, 1H), 2.20-2.03 (m, 1H); ESI-MS(m/z): 567.0 [M + H]⁺. |
| Ex- ample 148 | (E)-2,4-difluoro- N-(2-methoxy- 5-(4-((1-(4- oxopent-2- enoyl)pyrrolidin- 3-yl)amino) quinazolin-6- yl)pyridin-3-yl) benzene- sulfonamide | 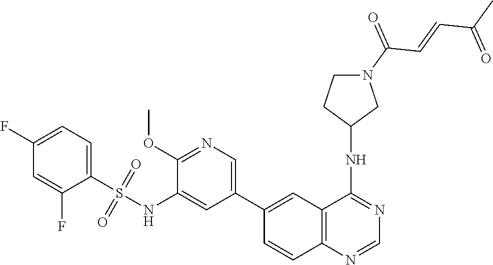 [SMILES: COc1ncc(-c2ccc3ncnc(NC4CCN(C(=O)/C=C/C(C)=O)C4)c3c2)cc1NS(=O)(=O)c1ccc(F)cc1F] | ¹H NMR (400 MHz, DMSO-d₆) δ 10.37 (s, 1H), 8.60 (s, 1H), 8.55 (d, J = 4.0 Hz, 1H), 8.48 (s, 1H), 8.34- 8.30 (s, 1H), 8.09-8.06 (m, 2H), 7.80 (dd, J = 4.0 Hz, 8.0 Hz, 1H), 7.76-7.70 (m, 1H), 7.61-7.56 (m, 1H), 7.25-7.16 (m, 2H), 6.76 (t, J = 16.0 Hz, 1H), 4.96-4.80 (m, 1H), 4.19-4.15 (m, 1H), 3.93-3.83 (m, 2H), 3.75-3.55 (m, 5H), 2.36 (d, J = 8.0 Hz, 3H), 2.22-2.11 (m, 1H); ESI-MS(m/z): 609.0 [M + H]⁺. |
| Ex- ample 149 | 4-fluoro-N-(2- methoxy-5-(4- (8-((E)-4- oxopent-2- enoyl)-3,8- diazabicyclo [3.2.1]octan-3- yl)quinazolin-6- yl)pyridin-3-yl) benzene- sulfonamide | 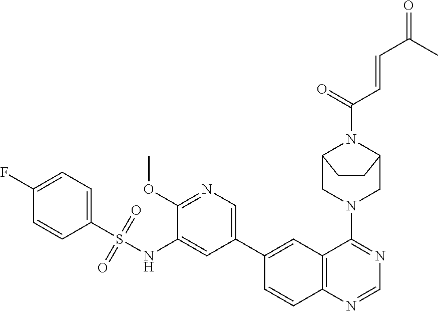 [SMILES: COc1ncc(-c2ccc3ncnc(N4CC5CCC(C4)N5C(=O)/C=C/C(C)=O)c3c2)cc1NS(=O)(=O)c1ccc(F)cc1] | ¹H NMR (400 MHz, DMSO-d₆) δ 10.10 (s, 1H), 8.61 (s, 1H), 8.42 (s, 1H), 8.07-8.03 (m, 2H),7.96 (s, 1H), 7.89 (d, J = 8.0 Hz, 1H), 7.85-7.80 (m, 2H), 7.44-7.39 (m, 3H), 6.84 (d, J = 16.0 Hz, 1H), 4.80-4.75 (m, 2H), 4.55-4.46 (m, 2H), 3.68 (s, 3H), 2.63-3.57 (m, 2H), 2.40 (s, 3H), 1.97-1.91 (m, 1H),1.84-1.80 (m, 3H); ESI-MS(m/z): 617.0 [M + H]⁺. |
| Ex- ample 150 | (E)-2-fluoro-N- (2-methoxy-5- (4-(2-(4- oxopent-2- enoyl)-2,7- diazaspiro[3.5] nonan-7- yl)quinazolin-6- yl)pyridin-3-yl) benzene- sulfonamide | 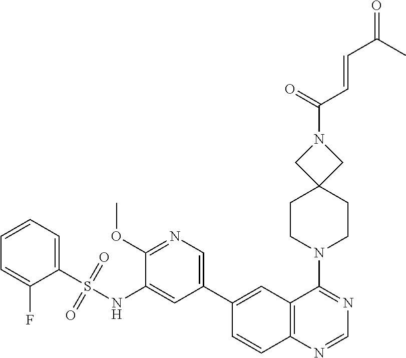 [SMILES: COc1ncc(-c2ccc3ncnc(N4CCC5(CC4)CN(C(=O)/C=C/C(C)=O)C5)c3c2)cc1NS(=O)(=O)c1ccccc1F] | ¹H NMR (400 MHz, DMSO-d₆) δ 10.20 (s, 1H), 8.63 (s, 1H), 8.42 (d, J = 4 Hz, 1H), 8.11-8.01 (m, 2H), 7.98 (d, J = 4 Hz, 1H), 7.89 (d, J = 8 Hz, 1H), 7.73-7.70 (m, 2H), 7.46-7.43 (m, 1H), 7.35-7.33(m, 1H), 6.93 (d, J = 16 Hz, 1H), 6.74 (d, J = 16 Hz, 1H), 4.15 (s, 2H), 3.84-3.70 (m, 6H), 3.67 (s, 3H), 2.35 (s, 3H), 1.96 (t, J = 8 Hz, 4H); ESI-MS(m/z): 631.0 [M + H]⁺. |
| Ex- ample 151 | 2-fluoro-N-(2- methoxy-5-(4- (8-((E)-4- oxopent-2- enoyl)-3,8- diazabicyclo [3.2.1]octan-3- yl)quinazolin-6- yl)pyridin-3-yl) benzene- sulfonamide | 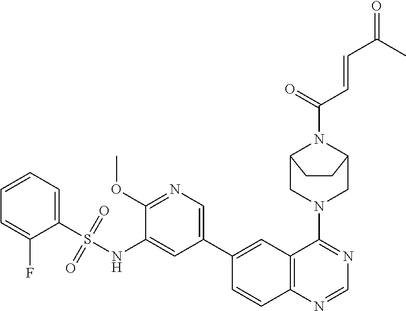 [SMILES: COc1ncc(-c2ccc3ncnc(N4CC5CCC(C4)N5C(=O)/C=C/C(C)=O)c3c2)cc1NS(=O)(=O)c1ccccc1F] | ¹H NMR (400 MHz, DMSO-d₆) δ 10.25 (s, 1H), 8.60 (s, 1H), 8.44 (s, 1H), 8.08-8.05 (m, 2H), 8.03 (s, 1H), 7.88 (d, J = 8 Hz, 1H), 7.74-7.69 (m, 2H), 7.46-7.33 (m, 3H), 6.85 (d, J = 16 Hz, 1H), 4.78-4.74 (m, 2H), 4.63- 4.48 (m, 2H), 3.65 (s, 3H), 3.63-3.56 (m, 2H), 2.39 (s, 3H), 1.82-1.77 (m, 4H); ESI-MS(m/z): 617.0 [M + H]⁺. |
| Ex- ample 152 | (S,E)-2-fluoro- N-(2-methoxy- 5-(4-(2-methyl- 4-(4-oxopent-2- enoyl)piperazin- 1-yl)quinazolin- 6-yl)pyridin-3- yl)benzene- sulfonamide | 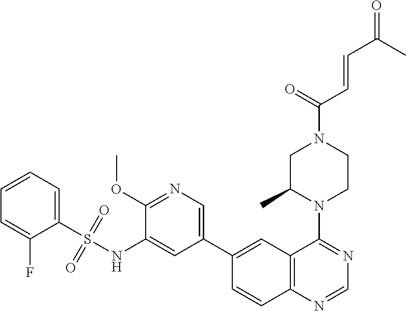 [SMILES: COc1ncc(-c2ccc3ncnc(N4CCN(C(=O)/C=C/C(C)=O)C[C@@H]4C)c3c2)cc1NS(=O)(=O)c1ccccc1F] | ¹H NMR (400 MHz, DMSO-d₆) δ 8.64 (s, 1H), 8.05 (s, 1H), 7.97-7.95 (m, 2H),7.89-7.86 (m, 1H), 7.77- 7.73 (m, 2H), 7.51-7.44 (m, 2H), 7.38 (d, J = 16.0 Hz, 1H), 7.30-7.21 (m, 2H), 6.76 (dd, J = 12.0 Hz, 16.0 Hz, 1H), 4.73-4.70 (m, 1H), 4.41- 4.25 (m, 1H), 4.20-3.96 (m, 2H), 3.74 (s, 3H), 3.67-3.46 (m, 2H), 3.21-3.10 (m, 1H), 2.38 (d, J = 4.0 Hz, 3H), 1.31-1.28 (m, 3H); ESI- MS(m/z): 605.0 [M + H]⁺. |
| Ex- ample 153 | (S,E)-4-fluoro- N-(2-methoxy- 5-(4-(2-methyl- 4-(4-oxopent-2- enoyl)piperazin- 1-yl)quinazolin- 6-yl)pyridin-3- yl)benzene- sulfonamide | 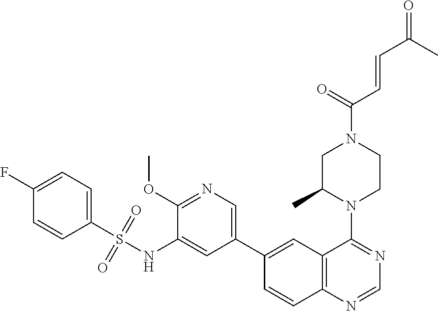 [SMILES: COc1ncc(-c2ccc3ncnc(N4CCN(C(=O)/C=C/C(C)=O)C[C@@H]4C)c3c2)cc1NS(=O)(=O)c1ccc(F)cc1] | ¹H NMR (400 MHz, DMSO-d₆) δ 10.12 (s, 1H), 8.67 (s, 1H), 8.45-8.42 (m, 1H), 8.11-8.04 (m, 2H), 8.01- 7.98 (m, 1H), 7.92 (d, J = 8.0 Hz, 1H), 7.85-7.80 (m, 2H), 7.53-7.43 (m, 1H), 7.43-7.36 (m, 2H), 6.80- 6.71 (m, 1H), 4.85-4.71 (m, 1H), 4.45-3.97 (m, 3H), 3.68 (s, 3H), 3.65-3.47 (m, 2H), 3.23-3.12 (m, 1H), 2.37 (d, J = 8.0 Hz, 3H), 1.33 (t, J = 8.0 Hz, 3H); ESI-MS(m/z): 605.0 [M + H]⁺. |
| Ex- ample 154 | (S,E)-2,4,6- trifluoro-N-(2- methoxy-5-(4- (2-methyl-4-(4- oxopent-2- enoyl)piperazin- 1-yl)quinazolin- 6-yl)pyridin-3- yl)benzene- sulfonamide | 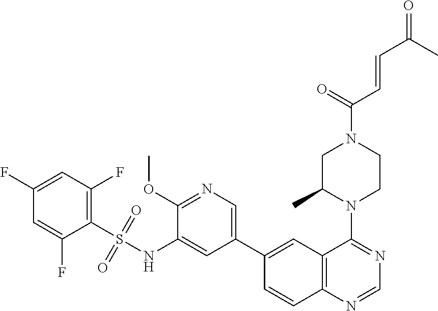 [SMILES: COc1ncc(-c2ccc3ncnc(N4CCN(C(=O)/C=C/C(C)=O)C[C@@H]4C)c3c2)cc1NS(=O)(=O)c1c(F)cc(F)cc1F] | ¹HNMR (400 MHz, DMSO-d₆) δ 10.65 (s, 1H), 8.67 (s, 1H), 8.48 (s, 1H), 8.13-8.00 (m, 3H), 7.92 (d, J = 8 Hz, 1H), 7.53-7.33 (m, 3H), 6.75 (dd, J₁ = 16, J₂ = 8 Hz, 1H), 4.79 (m, 1H), 4.41-4.19 (m, 2H), 4.20-3.97 (m, 1H), 3.69 (s, 3H), 3.62-3.44 (m, 2H), 3.25-3.11 (m, 1H), 2.37 (s, 3H), 1.32 (t, J = 8 Hz, 3H); ESI-MS(m/z): 641.0 [M + H]⁺. |
| Ex- ample 155 | 2,4,6-trifluoro- N-(2-methoxy- 5-(4-(8-((E)-4- oxopent-2- enoyl))-3,8- diazabicyclo [3.2.1]octyl-3- yl)quinazolin-6- yl)pyridin-3-yl) benzene- sulfonamide | 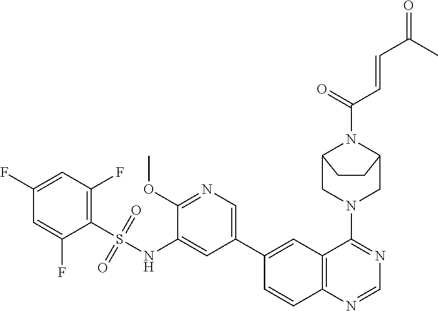 [SMILES: COc1ncc(-c2ccc3ncnc(N4CC5CCC(C4)N5C(=O)/C=C/C(C)=O)c3c2)cc1NS(=O)(=O)c1c(F)cc(F)cc1F] | ¹H NMR (400 MHz, DMSO-d₆) δ 10.66 (s, 1H), 8.61 (s, 1H), 8.45 (s, 1H), 8.13-7.97 (m, 3H), 7.89 (d, J = 8.8 Hz, 1H), 7.47-7.35 (m, 3H), 6.84 (d, J = 15.6 Hz, 1H), 4.83-4.71 (m, 2H), 4.51 (dd, J = 26.8, 12.0 Hz, 2H), 3.72-3.55 (m, 5H), 2.40 (s, 3H), 2.00-1.74 (m, 4H); ESI-MS(m/z): 653.0 [M + H]⁺. |
| Ex- ample 156 | 2,4,6-trifluoro- N-(5-(4-(8-(2- fluoroacryloyl)- 3,8- diazabicyclo [3.2.1]octan-3- yl)quinazolin-6- yl)-2- methoxypyridin- 3-yl) benzene- sulfonamide | 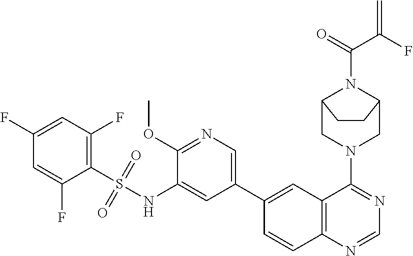 [SMILES: C=C(F)C(=O)N1C2CCC1CN(c1ncnc3ccc(-c4cnc(OC)c(NS(=O)(=O)c5c(F)cc(F)cc5F)c4)cc13)C2] | ¹H NMR (400 MHz, DMSO-d₆) δ 10.67 (s, 1H), 8.61 (s, 1H), 8.50 (s, 1H), 8.13-8.01 (m, 3H), 7.89 (d, J = 8.8 Hz, 1H), 7.47-7.37 (m, 2H), 5.47 (dd, J = 36.8, 3.6 Hz, 1H), 5.40-5.33 (m, 1H), 4.75-4.57 (m, 2H), 4.56- 4.48 (m, 2H), 3.71-3.55 (m, 5H), 1.96-1.72 (m, 4H); ESI-MS(m/z): 629.0 [M + H]⁺. |
| Ex- ample 157 | (E)-2,4-difluoro- N-(2-methoxy- 5-(4-(5-(4- oxopent-2- enoyl) hexahydro- pyrrolo[3,4- c]pyrrol-2(1H)- yl)quinazolin-6- yl)pyridin-3-yl) benzene- sulfonamide | 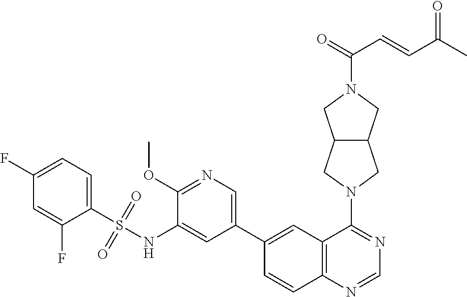 [SMILES: COc1ncc(-c2ccc3ncnc(N4CC5CN(C(=O)/C=C/C(C)=O)CC5C4)c3c2)cc1NS(=O)(=O)c1ccc(F)cc1F] | ¹H NMR (400 MHz, DMSO-d₆) δ 10.38 (s, 1H), 8.52-8.32 (m, 3H), 8.07-7.95 (m, 2H), 7.83-7.70 (m, 2H), 7.63-7.52 (m, 1H), 7.25-7.10 (m, 2H), 6.72 (d, J = 16.0 Hz, 1H), 4.31-4.17 (m, 2H), 4.04-3.89 (m, 3H), 3.79-3.70 (m, 2H), 3.66 (s, 3H), 3.53-3.42 (m, 1H), 3.21-3.02 (m, 2H), 2.33 (s, 3H); ESI-MS(m/z): 635.0 [M + H]⁺. |
| Ex- ample 158 | (E)-2,4-difluoro- N-(2-methoxy- 5-(4-(5-(4- oxopent-2- enoyl)-2,5- diazabicyclo [2.2.2]octan-2- yl)quinazolin-6- yl)pyridin-3-yl) benzene- sulfonamide | 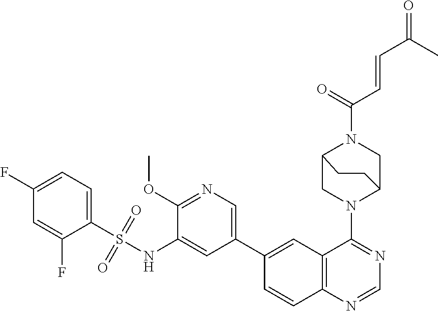 [SMILES: COc1ncc(-c2ccc3ncnc(N4CC5CCC4CN5C(=O)/C=C/C(C)=O)c3c2)cc1NS(=O)(=O)c1ccc(F)cc1F] | ¹H NMR (400 MHz, DMSO-d₆) δ 10.34 (s, 1H), 8.65 (s, 1H), 8.50-8.47 (m, 1H), 8.33 (d, J = 8.0 Hz, 1H), 8.13 (d, J = 16.0 Hz, 1H), 8.04-8.01 (m, 1H), 7.86 (d, J = 8.0 Hz, 1H), 7.79-7.72 (m, 1H), 7.61-7.54 (m, 1H), 7.40 (d, J = 16.0 Hz, 1H), 7.24- 7.16 (m, 1H), 6.83-6.69 (m, 1H), 5.23 (s, 1H), 4.80-4.58 (m, 1H), 4.56-4.43 (m, 1H), 4.31-4.23 (m, 1H), 4.14-3.99 (m, 1H), 3.83-3.69 (m, 1H), 3.67-3.64 (m, 3H), 2.36- 2.34 (m, 3H), 2.28-2.18 (m, 1H), 2.03-1.86 (s, 3H); ESI-MS(m/z): 635.0 [M + H]⁺. |
| Ex- ample 159 | (E)-2,4-difluoro- N-(5-(4-(4-(4- oxopent-2- enoyl)piperazin- 1-yl)quinazolin- 6-yl)-2-(2,2,2- trifluoroethoxy) pyridin-3-yl) benzene- sulfonamide | 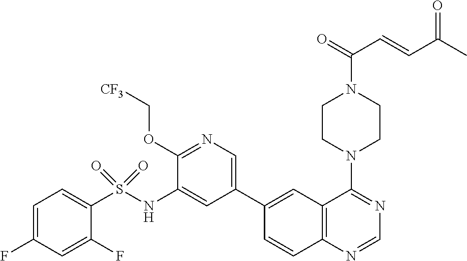 [SMILES: CC(=O)/C=C/C(=O)N1CCN(c2ncnc3ccc(-c4cnc(OCC(F)(F)F)c(NS(=O)(=O)c5ccc(F)cc5F)c4)cc23)CC1] | ¹H NMR (400 MHz, DMSO-d₆) δ 10.58 (s, 1H), 8.67 (s, 1H), 8.47 (s, 1H), 8.16-8.08 (m, 3H), 7.93 (d, J = 8.0 Hz, 1H), 7.84-7.77 (m, 1H), 7.50-7.42 (m, 2H), 7.22-7.15 (m, 1H), 6.77-6.70 (m, 1H), 4.94-4.86 (m, 2H), 3.93-3.86 (m, 6H), 3.83- 3.78 (m, 2H), 2.37 (s, 3H); ESI- MS(m/z): 677.0 [M + H]⁺. |
| Ex- ample 160 | (E)-N-(2- methoxy-5-(4- (4-(4-oxopent-2- enoyl)piperazin- 1-yl)quinazolin- 6-yl)pyridin-3- yl)-2,4- dimethyl- thiazole- 5-sulfonamide | 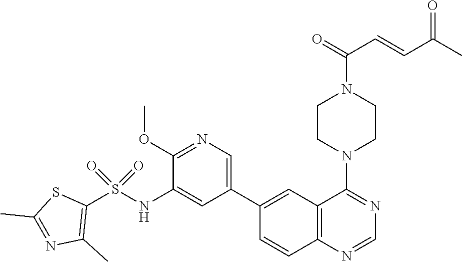 [SMILES: COc1ncc(-c2ccc3ncnc(N4CCN(C(=O)/C=C/C(C)=O)CC4)c3c2)cc1NS(=O)(=O)c1sc(C)nc1C] | ¹H NMR (400 MHz, DMSO-d₆) δ 10.39 (s, 1H), 8.68 (s, 1H), 8.53 (d, J = 4.0 Hz, 1H), 8.14-8.09 (m, 2H), 8.04 (d, J = 4.0 Hz, 1H), 7.94 (d, J = 8.0 Hz, 1H), 7.46 (d, J = 16.0 Hz, 1H), 6.74 (d, J = 16.0 Hz, 1H), 3.92- 3.80 (m, 8H),3.74 (s, 3H), 2.60 (s, 3H), 2.38 (s, 6H); ESI-MS(m/z): 608.0 [M + H]⁺. |

### Example 161

Preparation of (E)-4-fluoro-N-(2-methoxy-5-(4-(2-(4-oxopent-2-enoyl)-2,7-diazaspiro[3.5]nonan-7-yl) quinazolin-6-yl)pyridin-3-yl)benzenesulfonamide

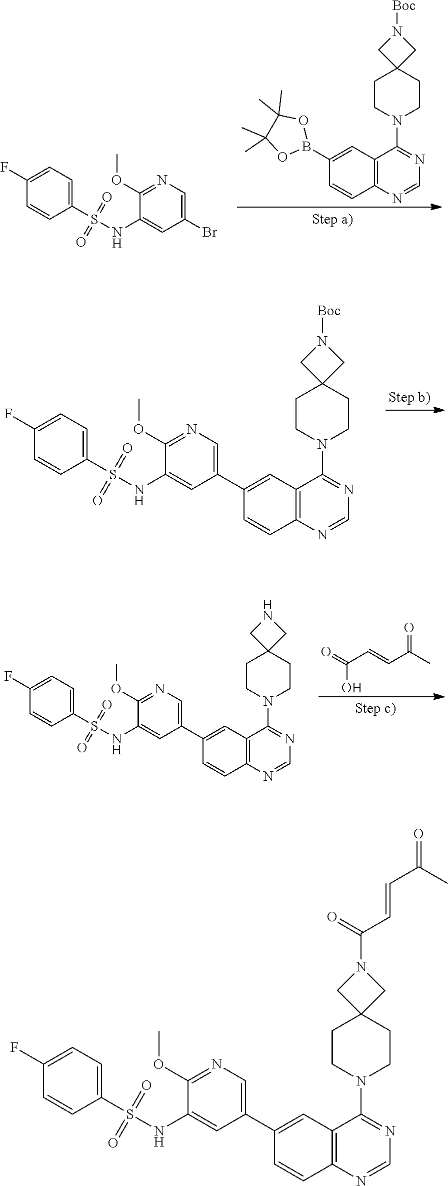

### Step a): Preparation of tert-butyl 7-(6-(5-((4-fluorophenyl) sulfonamido)-6-methoxypyridin-3-yl) quinazolin-4-yl)-2,7-diazaspiro[3.5]nonane-2-carboxylate

N-(5-bromo-2-methoxypyridin-3-yl)-4-fluorobenzenesulfonamide (100 mg, 0.277 mmol), tert-butyl 7-(6-(4,4,5,5-tetramethyl-1,3,2-dioxaborolan-2-yl) quinazolin-4-yl)-2,7-diazaspiro[3.5]nonane-2-carboxylate (133 mg, 0.277 mmol), Pd(dppf)Cl₂ (20 mg, 0.027 mmol) and cesium carbonate (180 mg, 0.554 mmol) were added to dioxane/water mixed solvent (4.4 mL, v/v=10:1). After the addition was completed, in the presense of protective nitrogen, the reaction mixture was heated to 100° C. and stirred for 3 h. Upon completion of the reaction, the reaction was quenched by adding water (10 mL). The reaction mixture was extracted by adding dichloromethane (10 mL×3). The organic phases were combined, dried over anhydrous sodium sulfate and filtered. The filtrate was concentrated under reduced pressure. The crude product obtained was purified by silica gel column chromatography (Eluent: dichloromethane/methanol=1/0 to 20/1). Tert-butyl 7-(6-(5-((4-fluorophenyl)sulfonamido)-6-methoxypyridin-3-yl)quinazolin-4-yl)-2,7-diazaspiro[3.5]nonane-2-carboxylate was obtained, yield: 63.9%; ESI-MS (m/z): 635.6 [M+H]⁺.

### Step b): Preparation of N-(5-(4-(2,7-diazaspiro[3.5]nonan-7-yl)quinazolin-6-yl)-2-methoxypyridin-3-yl)-4-fluorobenzenesulfonamide trifluoroacetate

Tert-butyl 7-(6-(5-((4-fluorophenyl)sulfonamido)-6-methoxypyridin-3-yl)quinazolin-4-yl)-2,7-diazaspiro[3.5]nonane-2-carboxylate (120 mg, 0.190 mmol) and dichloromethane (4 mL) were added to the reaction flask. With the reaction solution cooled to 0° C. trifluoroacetic acid (2 mL) was slowly added. After the addition was completed, the reaction mixture was naturally heated to room temperature and reacted for 0.5 h. Upon completion of the reaction, the reaction mixture was concentrated under reduced pressure. N-(5-(4-(2,7-diazaspiro[3.5]nonan-7-yl)quinazolin-6-yl)-2-methoxypyridin-3-yl)-4-fluorobenzenesulfonamide trifluoroacetate was obtained; ESI-MS (m/z): 535.2 [M+H]⁺.

### Step c): Preparation of (E)-4-fluoro-N-(2-methoxy-5-(4-(2-(4-oxopent-2-enoyl)-2,7-diazaspiro[3.5]nonan-7-yl)quinazolin-6-yl)pyridin-3-yl)benzenesulfonamide

N-(5-(4-(2,7-diazaspiro[3.5]nonan-7-yl)quinazolin-6-yl)-2-methoxypyridin-3-yl)-4-fluorobenzenesulfonamide trifluoroacetate (150 mg, 0.230 mmol) and tetrahydrofuran (2 mL) were added to the reaction flask. With the reaction solution cooled to −78° C., DIPEA (180 mg, 1.380 mmol), (E)-4-oxopent-2-enoic acid (26 mg, 0.230 mmol) and 50% T₃P ethyl acetate solution (150 mg, 0.230 mmol) were added successively. After the addition was completed, the reaction mixture was kept at −78° C. and stirred for 1 h. Upon completion of the reaction, the reaction solution was diluted by adding acetonitrile (1 mL) and purified by Prep-HPLC (Method 2). (E)-4-fluoro-N-(2-methoxy-5-(4-(2-(4-oxopent-2-enoyl)-2,7-diazaspiro[3.5]nonan-7-yl)quinazolin-6-yl)pyridin-3-yl)benzenesulfonamide was obtained, yield: 62.4%; ¹H NMR (400 MHz, DMSO-d₅) δ 10.04 (s, 1H), 8.64 (s, 1H), 8.40 (d, J=4.0 Hz, 1H), 8.08-8.01 (m, 2H), 7.96 (d, J=4.0 Hz, 1H), 7.90 (d, J=8.0 Hz, 1H), 7.85-7.81 (m, 2H), 7.45-7.39 (m, 2H), 6.93 (d, J=16.0 Hz, 1H), 6.74 (d, J=16.0 Hz, 1H), 4.15 (s, 2H), 3.80-3.70 (m, 9H), 2.35 (s, 3H), 1.98-1.95 (m, 4H); ESI-MS(m/z): 631.0 [M+H]⁺.

Referring to the preparation method of Example 161 and using the corresponding raw materials, the compounds in the following examples were obtained.

| Number | Name | Structure | ¹H NMR and MS |
|---|---|---|---|
| Example 162 | 3,5-difluoro- N-(2-methoxy- 5-(4-(8-((E)-4- oxopent-2- enoyl)-3,8- diazabicyclo [3.2.1]octan-3- yl)quinazolin- 6- yl)pyridin-3- yl)pyridine-4- sulfonamide | 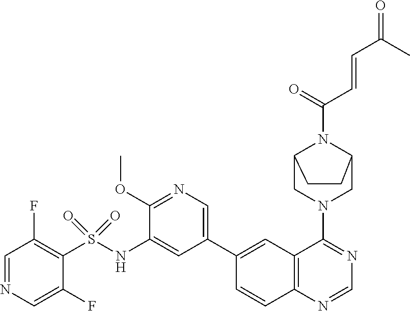 [SMILES: COc1ncc(-c2ccc3ncnc(N4CC5CCC(C4)N5C(=O)/C=C/C(C)=O)c3c2)cc1NS(=O)(=O)c1c(F)cncc1F] | ¹H NMR (400 MHz, DMSO-d₆) δ 11.20 (brs, 1H), 8.76 (s, 2H), 8.61 (s, 1H), 8.49 (s, 1H), 8.18-7.98 (m, 3H), 7.89 (d, J = 8.0 Hz, 1H), 7.41 (d, J = 16.0 Hz, 1H), 6.85 (d, J = 16.0 Hz, 1H), 4.83-4.71 (m, 2H), 4.59-4.45 (m, 2H), 3.69-3.50 (m, 5H), 2.40 (s, 3H), 1.98-1.74 (m, 4H); ESI-MS (m/z): 636.0 [M + H]⁺. |
| Example 163 | (E)-3,5- difluoro-N- (2-methoxy-5- (4-(2-(4- oxopent- 2-enoyl)-2,7- diazaspiro[3.5] nonan-7-yl) quinazolin-6- yl)pyridin-3- yl)pyridine-4- sulfonamide | 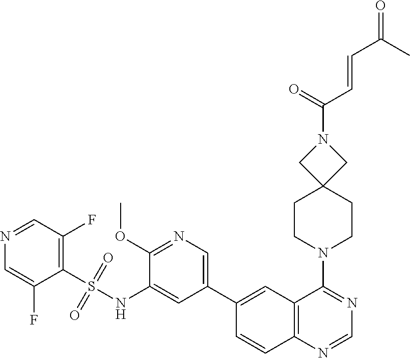 [SMILES: COc1ncc(-c2ccc3ncnc(N4CCC5(CC4)CN(C(=O)/C=C/C(C)=O)C5)c3c2)cc1NS(=O)(=O)c1c(F)cncc1F] | ¹H NMR (400 MHz, DMSO-d₆) δ 11.20 (brs, 1H), 8.68 (s, 2H), 8.63 (s, 1H), 8.32 (s, 1H), 8.10-8.00 (m, 2H), 8.00-7.92 (m, 1H), 7.89 (d, J = 8.0 Hz, 1H), 6.94 (d, J = 16.0 Hz, 1H), 6.74 (d, J = 16.0 Hz, 1H), 4.15 (s, 2H), 3.80 (s, 2H), 3.78-3.67 (m, 4H), 3.65 (s, 3H), 2.35 (s, 3H), 2.02-1.86 (m, 4H); ESI-MS (m/z): 650.0 [M + H]⁺. |
| Example 164 | (S,E)-3,5- difluoro-N-(2- methoxy-5-(4- (2-methyl- 4-(4-oxopent- 2-enoyl) piperazin-1- yl)quinazolin- 6- yl)pyridin-3- yl)pyridine-4- sulfonamide | 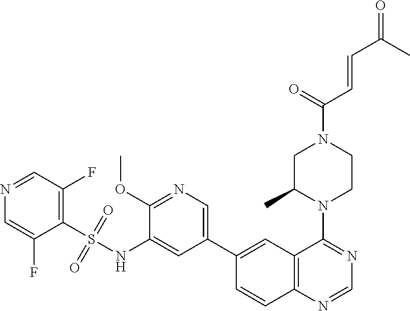 [SMILES: COc1ncc(-c2ccc3ncnc(N4CCN(C(=O)/C=C/C(C)=O)C[C@@H]4C)c3c2)cc1NS(=O)(=O)c1c(F)cncc1F] | ¹H NMR (400 MHz, DMSO-d₆) δ 8.65 (s, 1H), 8.45 (s, 1H), 7.98-7.87 (m, 3H), 7.72 (d, J = 4.0 Hz, 1H), 7.45 (dd, J₁ = 48.0 Hz, J₂ = 16.0, 1H), 7.20-7.01 (m, 3H), 6.75 (dd, J₁ = 16.0 Hz, J₂ = 8.0. Hz, 1H), 4.81-4.67 (m, 1H), 4.50-3.94 (m, 3H), 3.74 (s, 3H), 3.69-3.45 (m, 2H), 3.25-3.09 (m, 1H), 2.38 (d, J = 4.0 Hz, 3H), 1.30 (t, J = 4.0 Hz, 3H); ESI-MS (m/z): 624.0 [M + H]⁺. |
| Example 165 | (E)-5-chloro- N-(2-methoxy- 5-(4-(4- (4-oxopent-2- enoyl) piperazin-1-yl) quinazolin-6- yl)pyridin-3- yl)thiazole-2- sulfonamide | 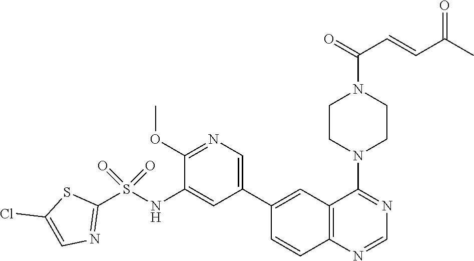 [SMILES: COc1ncc(-c2ccc3ncnc(N4CCN(C(=O)/C=C/C(C)=O)CC4)c3c2)cc1NS(=O)(=O)c1ncc(Cl)s1] | ¹H NMR (400 MHz, DMSO-d₆) δ 11.03 (s, 1H), 8.68 (s, 1H), 8.48 (s, 1H), 8.20-8.08 (m, 4H), 7.93 (d, J = 8.8 Hz, 1H), 7.44 (d, J = 15.6 Hz, 1H), 6.73 (d, J = 16.0 Hz, 1H), 3.97- 3.77 (m, 8H), 3.76 (s, 3H), 2.36 (s, 3H); ESI-MS (m/z): 614.0 [M + H]⁺. |

### Example 166

### Preparation of (E)-N-(2,4-difluorophenyl)-2-methoxy-5-(4-(4-(4-oxopent-2-enoyl)piperazin-1-yl)quinazolin-6-yl)pyridine-3-sulfonamide

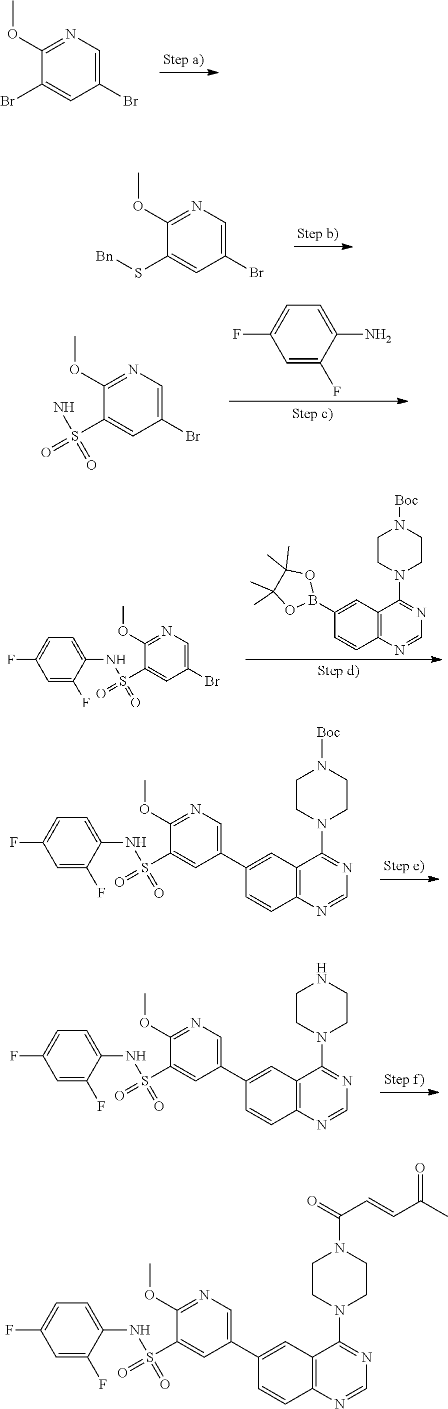

### Step a): Preparation of 3-(benzylthio)-5-bromo-2-methoxypyridine

Sodium hydride (720 mg, 17.994 mmol) was added to three-necked bottle. In the presense of protective nitrogen, N,N-dimethylformamide (50 mL) was added to the system. The reaction system was cooled to 0° C., with benzylthiol (1.2 g, 14.994 mmol) slowly added, stirred and reacted for 10 min. Then with 3,5-dibromo-2-methoxypyridine (4.0 g, 14.994 mmol) added, the reaction mixture was kept at room temperature and reacted for 3 h. Upon completion of the reaction, the reaction solution was poured into iced water (45 mL) and extracted with ethyl acetate (80 mL). The organic phases were combined, washed with saturated sodium chloride (80 mL×3), dried over anhydrous sodium sulfate and filtered. The filtrate was concentrated under reduced pressure. The crude product obtained was purified by silica gel column chromatography (Eluent: petroleum ether/ethyl acetate=1/0 to 10/1), 3-(Benzylthio)-5-bromo-2-methoxypyridine was obtained, yield: 68.8%; ¹H NMR (400 MHz, DMSO-d₆) δ 8.04 (d, J=2.2 Hz, 1H), 7.77 (d, J=2.2 Hz, 1H), 7.43-7.37 (m, 2H), 7.36-7.29 (m, 2H), 7.29-7.23 (m, 1H), 4.29 (s, 2H), 3.89 (s, 3H); ESI-MS(m/z): 309.9 [M+H]⁺.

### Step b): Preparation of 5-bromo-2-methoxypyridine-3-sulfonyl chloride

3-(Benzylthio)-5-bromo-2-methoxypyridine (600 mg, 1.934 mmol), acetic acid (6 mL) and water (2 mL) were added to the reaction flask, with N-chlorosuccinimide (1.0 g, 7.489 mmol) added in portions. After the addition was completed, the reaction mixture was kept at room temperature and reacted for 3 h. Upon completion of the reaction, the reaction mixture was concentrated under reduced pressure. The residue was dissolved by adding ethyl acetate (25 mL), washed with water (25 mL×3), dried over anhydrous sodium sulfate and filtered. The filtrate was concentrated under reduced pressure. 5-Bromo-2-methoxypyridine-3-sulfonyl chloride was obtained. The product can be used directly in the next reaction without purification.

### Step c): Preparation of 5-bromo-N-(2,4-difluorophenyl)-2-methoxypyridine-3-sulfonamide

2,4-Difluoroaniline (248 mg, 1.921 mmol), 4-dimethylaminopyridine (24 mg, 0.194 mmol) and pyridine (456 mg, 5.764 mmol) were added to the reaction flask where 5-bromo-2-methoxypyridine-3-sulfonyl chloride (500 mg, crude product) was placed. In the presense of protective nitrogen, the reaction mixture was kept at room temperature and reacted for 15 h. Upon completion of the reaction, the reaction was quenched by adding saturated aqueous ammonium chloride solution (40 mL), and the reaction mixture was extracted with dichloromethane (30 mL×2). The organic phases were combined, dried over anhydrous sodium sulfate and filtered. The filtrate was concentrated under reduced pressure. The crude product obtained was slurried with petroleum ether/ethyl acetate mixed solvent (5 mL, v/v=3/1) and filtered. 5-Bromo-N-(2,4-difluorophenyl)-2-methoxypyridine-3-sulfonamide was obtained, two-step yield: 92.8%; ESI-MS(m/z): 378.9 [M+H]⁺.

### Step d): Preparation of tert-butyl 4-(6-(5-(N-(2,4-difluorophenyl)sulfamoyl)-6-methoxypyridin-3-yl)quinazolin-4-yl)piperazine-1-carboxylate

5-Bromo-N-(2,4-difluorophenyl)-2-methoxypyridine-3-sulfonamide (170 mg, 0.448 mmol), tert-butyl 4-(6-(4,4,5,5-tetramethyl-1,3,2-dioxaborolan-2-yl)quinazolin-4-yl)piperazine-1-carboxylate (200 mg, 0.454 mmol), Pd(dppf)Cl₂ (33 mg, 0.045 mmol), cesium carbonate (440 mg, 1.352 mmol) and dioxane (15 mL)/water (5 mL) mixed solvent were added to the reaction flask. After the addition was completed, in the presense of protective nitrogen, the reaction mixture was kept at 100° C. and stirred for 5 h. Upon completion of the reaction, the reaction mixture was cooled to room temperature, with the reaction quenched by adding saturated ammonium chloride (30 mL), extracted with ethyl acetate (30 mL×2). The organic phases were combined, dried over anhydrous sodium sulfate and filtered. The filtrate was concentrated under reduced pressure. The crude product obtained was purified by silica gel column chromatography (Eluent: dichloromethane/methanol=1/0 to 35/1). Tert-butyl 4-(6-(5-(N-(2,4-difluorophenyl)sulfamoyl)-6-methoxypyridin-3-yl)quinazolin-4-yl)piperazine-1-carboxylate was obtained, yield: 85.3%; ESI-MS(m/z): 613.5 [M+H]⁺.

### Step e): Preparation of N-(2,4-difluorophenyl)-2-methoxy-5-(4-(piperazin-1-yl)quinazolin-6-yl)pyridine-3-sulfonamide trifluoroacetate

Tert-butyl 4-(6-(5-(N-(2,4-difluorophenyl)sulfamoyl)-6-methoxypyridin-3-yl)quinazolin-4-yl)piperazine-1-carboxylate (331 mg, 0.540 mmol) was dissolved in dichloromethane (9 mL). Under the ice bath condition, TFA (3 mL) was slowly added dropwise. After the addition was completed, the reaction mixture was stirred at room temperature for 1 h. Upon completion of the reaction, the reaction mixture was concentrated under reduced pressure. The crude product obtained was purified by slurrying with methyl tert-butyl ether (10 mL) for 0.5 h, filtered and vacuum dried. N-(2,4-difluorophenyl)-2-methoxy-5-(4-(piperazin-1-yl)quinazolin-6-yl)pyridine-3-sulfonamide trifluoroacetate was obtained, yield: 81.4%; ESI-MS(m/z): 513.0 [M+H]⁺.

### Step f): Preparation of (E)-N-(2,4-difluorophenyl)-2-methoxy-5-(4-(4-(4-oxopent-2-enoyl)piperazin-1-yl)quinazolin-6-yl)pyridine-3-sulfonamide

N-(2,4-difluorophenyl)-2-methoxy-5-(4-(piperazin-1-yl)quinazolin-6-yl)pyridine-3-sulfonamide trifluoroacetate (100 mg, 0.160 mmol), (E)-4-oxopent-2-enoic acid (27 mg, 0.234 mmol), DIPEA (126 mg, 0.976 mmol) and tetrahydrofuran (2 mL) were added to the reaction flask. At −78° C., 50% 1-propylphosphonic anhydride ethyl acetate solution (149 mg, 0.234 mmol) was slowly added to the system. The reaction solution was kept at −78° C. and reacted for 1 h. Upon completion of the reaction, the reaction solution was diluted by adding acetonitrile (I mL) and purified by Prep-HPLC (Method 2). (E)-N-(2,4-difluorophenyl)-2-methoxy-5-(4-(4-(4-oxopent-2-enoyl)piperazin-1-yl)quinazolin-6-yl)pyridine-3-sulfonamide was obtained, yield: 27.3%; ¹H NMR (400 MHz, DMSO-d₆) δ 10.20 (s, 1H), 8.88 (d, J=2.4 Hz, 1H), 8.66 (s, 1H), 8.30 (d, J=2.8 Hz, 1H), 8.15 (d, J=2.0 Hz, 1H), 8.09 (dd, J=8.8, 2.0 Hz, 1H), 7.90 (d, J=8.8 Hz, 1H), 7.45 (d, J=16.0 Hz, 1H), 7.38-7.29 (m, 1H), 7.27-7.19 (m, 1H), 7.07-6.98 (m, 1H), 6.74 (d, J=16.0 Hz, 1H), 4.01 (s, 3H), 3.95-3.74 (m, 8H), 2.37 (s, 3H); ESI-MS(m/z): 609.0 [M+H]⁺.

### Example 167

### Preparation of 2,4-difluoro-N-(5-(4-(4-(3-hydroxy-2-methylenebutanoyl)piperazin-1-yl)quinazolin-6-yl)-2-methoxypyridin-3-yl)benzenesulfonamide

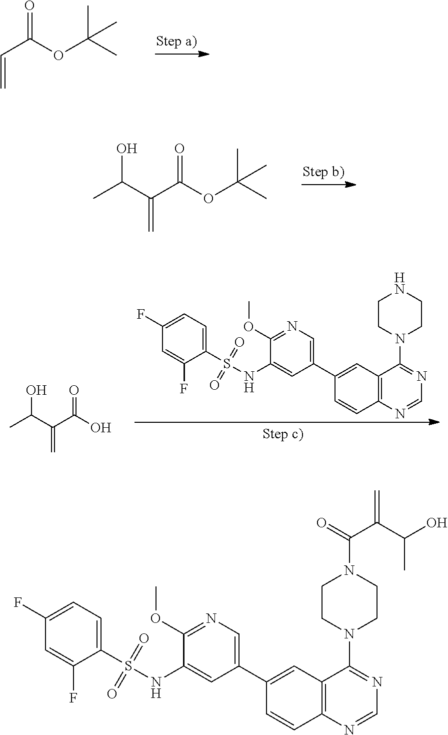

### Step a): Preparation of tert-butyl 3-hydroxy-2-methylenebutanoate

Triethylenediamine (6.4 g, 57.056 mmol), dioxane (10 mL), water (10 mL) and tert-butyl acrylate (14.6 g, 113.672 mmol) were placed in sealed tube reactor. With acetaldehyde (22.5 mL, 113.672 mmol, 5 M tetrahydrofuran solution) added, the reaction solution was kept at room temperature and reacted for 48 h. Upon completion of the reaction, water (30 mL) and methyl tert-butyl ether (30 mL) were added to the reaction solution. After that, the aqueous phase was extracted with methyl tert-butyl ether (30 mL×2). The organic phases were combined, washed with saturated aqueous ammonium chloride solution (40 mL) and saturated sodium chloride (40 mL×2) successively, dried over anhydrous sodium sulfate and filtered. The filtrate was concentrated under reduced pressure. The crude product obtained was purified by silica gel column chromatography (Eluent: petroleum ether/ethyl acetate=1/0 to 10/1). Tert-butyl 3-hydroxy-2-methylenebutanoate was obtained, yield: 17.4%; ¹H NMR (400 MHz, CDCl₃) δ 6.11 (s, 1H), 5.72 (s, 1H), 4.64-4.51 (m, 1H), 1.52 (s, 9H), 1.37 (d, J=6.8 Hz, 3H).

### Step b): Preparation of 3-hydroxy-2-methylenebutanoic acid

Tert-butyl 3-hydroxy-2-methylenebutanoate (200 mg, 1.161 mmol) was dissolved in dichloromethane (3 mL). Under the ice bath condition, TFA (1 mL) was slowly added dropwise. After the addition was completed, the reaction mixture was stirred at room temperature for 1 h, then reacted for 0.5 h at 40° C. Upon completion of the reaction, the reaction mixture was concentrated under reduced pressure. The crude product obtained was dispersed in methyl tert-butyl ether (5 mL), concentrated under reduced pressure. 3-Hydroxy-2-methylenebutanoic acid was obtained, yield: 83.3%; ¹H NMR (400 MHz, CDCl₃) δ 6.42 (s, 1H), 5.99 (s, 1H), 4.81-4.64 (m, 1H), 1.45 (d, J=6.8 Hz, 3H).

### Step c): Preparation of 2,4-difluoro-N-(5-(4-(4-(3-hydroxy-2-methylenebutanoyl)piperazin-1-yl)quinazolin-6-yl)-2-methoxypyridin-3-yl)benzenesulfonamide

2,4-Difluoro-N-(2-methoxy-5-(4-(piperazin-1-yl)quinazolin-6-yl)pyridin-3-yl)benzenesulfonamide trifluoroacetate (100 mg, 0.160 mmol), 3-hydroxy-2-methylenebutyric acid (28 mg, 0.241 mmol), DIPEA (129 mg, 1.000 mmol) and tetrahydrofuran (2 mL) were added to the reaction flask. At −78° C., T₃P ethyl acetate solution (155 mg, 0.244 mmol, 50%) was slowly added to the system. The reaction solution was kept at −78° C. and reacted for 1 h. Upon completion of the reaction, the reaction solution was diluted by adding acetonitrile (0 mL), filtered and purified by Prep-HPLC (Method 2), 2,4-Difluoro-N-(5-(4-(4-(3-hydroxy-2-methylenebutanoyl)piperazin-1-yl)quinazolin-6-yl)-2-methoxypyridin-3-yl)benzenesulfonamide was obtained, yield: 6.2%; ¹H NMR (400 MHz, DMSO-d₆) δ 10.34 (s, 1H), 8.67 (s, 1H), 8.53-8.45 (m, 1H), 8.15-8.08 (m, 2H), 8.02 (d, J=2.4 Hz, 1H), 7.91 (d, J=8.8 Hz, 1H), 7.83-7.72 (m, 1H), 7.62-7.53 (m, 1H), 7.26-7.18 (m, 1H), 5.38 (s, 1H), 5.12-5.05 (m, 2H), 4.44-4.34 (m, 1H), 3.92-3.70 (m, 8H), 3.67 (s, 3H), 1.20 (d, J=6.4 Hz, 3H); ESI-MS(m/z): 611.0 [M+H]⁺.

### Example 168

### Preparation of 2,4-difluoro-N-(2-methoxy-5-(4-(4-(2-methylene-3-oxobutanoyl)piperazin-1-yl)quinazolin-6-yl)pyridin-3-yl)benzenesulfonamide

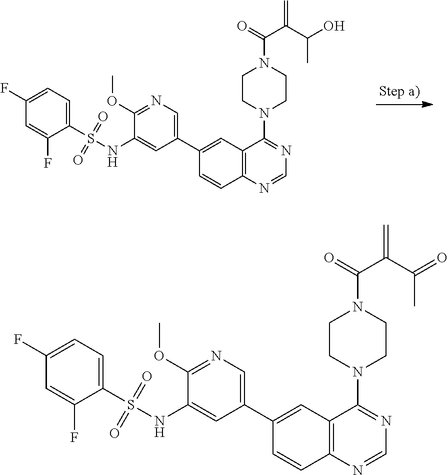 [SMILES: C=C(C(=O)N1CCN(c2ncnc3ccc(-c4cnc(OC)c(NS(=O)(=O)c5ccc(F)cc5F)c4)cc23)CC1)C(C)O ; C=C(C(C)=O)C(=O)N1CCN(c2ncnc3ccc(-c4cnc(OC)c(NS(=O)(=O)c5ccc(F)cc5F)c4)cc23)CC1]

2,4-Difluoro-N-(5-(4-(4-(3-hydroxy-2-methylenebutanoyl)piperazin-1-yl)quinazolin-6-yl)-2-methoxypyridin-3-yl)benzenesulfonamide (70 mg, 0.115 mmol) was dissolved in anhydrous acetonitrile (15 mL). With manganese dioxide (2999 mg, 34.500 mmol) added, in the presense of protective nitrogen, the reaction mixture was heated to reflux, stirred and reacted for 10 h. Upon completion of the reaction, the reaction mixture was cooled to room temperature and filtered. The filtrate was concentrated under reduced pressure. The crude product obtained was purified by Prep-HPLC (Method 2), 2,4-Difluoro-N-(2-methoxy-5-(4-(4-(2-methylene-3-oxobutanoyl)piperazin-1-yl)quinazolin-6-yl)pyridin-3-yl)benzenesulfonamide was obtained, yield: 24.0%; ¹H NMR (400 MHz, DMSO-d₆) δ 10.35 (s, 1H), 8.67 (s, 1H), 8.46 (s, 1H), 8.13-8.06 (m, 2H), 8.05-8.00 (m, 1H), 7.95-7.89 (m, 1H), 7.82-7.73 (m, 1H), 7.63-7.53 (m, 1H), 7.26-7.15 (m, 1H), 6.45 (s, 1H), 6.16 (s, 1H), 3.90-3.73 (m, 6H), 3.68 (s, 3H), 3.54-3.45 (m, 2H), 2.38 (s, 3H); ESI-MS(m/z): 609.0 [M+H]⁺.

### Example 169

### Preparation of (E)-2-chloro-4-fluoro-N-(2-methoxy-5-(4-(4-(4-oxopent-2-enoyl)piperazin-1-yl)quinazolin-6-yl)pyridin-3-yl)benzenesulfonamide

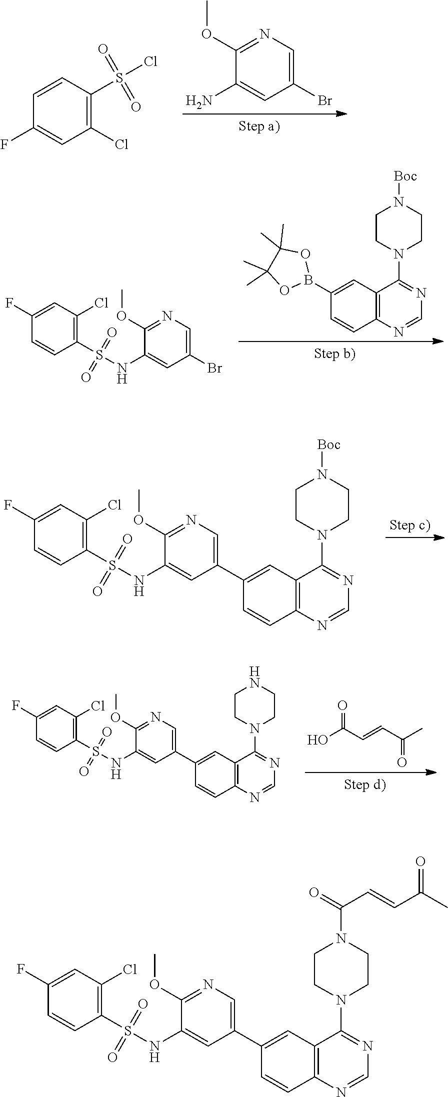

### Step a): Preparation of N-(5-bromo-2-methoxypyridin-3-yl)-2-chloro-4-fluorobenzenesulfonamide

2-Chloro-4-fluorobenzenesulfonyl chloride (350 mg, 1.528 mmol), 5-bromo-2-methoxypyridin-3-amine (310 mg, 1.528 mmol), 4-dimethylaminopyridine (19 mg, 0.153 mmol) and dichloromethane (6 mL) were added to the reaction flask. The reaction mixture was stirred to mix and cooled to 0° C., pyridine (242 mg, 3.056 mmol) was added dropwise. After the addition was completed, the reaction mixture was kept at room temperature and reacted for 12 h. Upon completion of the reaction, the reaction was quenched by adding water (30 mL). The reaction mixture was extracted with dichloromethane (60 mL×2). The organic phases were combined, dried over anhydrous sodium sulfate and filtered. The filtrate was concentrated under reduced pressure. The residue was purified by silica gel column chromatography (Eluent: petroleum ether/ethyl acetate=5/1 to 1/1). N-(5-bromo-2-methoxypyridin-3-yl)-2-chloro-4-fluorobenzenesulfonamide was obtained, yield: 69.5%, ESI-MS (m/z): 396.9 [M+H]⁺.

### Step b): Preparation of tert-butyl 4-(6-(5-((2-chloro-4-fluorophenyl)sulfonamido)-6-methoxypyridin-3-yl)pyrido[3,2-d]pyrimidin-4-yl)piperazine-1-carboxylate

N-(5-bromo-2-methoxypyridin-3-yl)-2-chloro-4-fluorobenzenesulfonamide (210 mg, 0.531 mmol), tert-butyl 4-(6-(4,4,5,5-tetramethyl-1,3,2-dioxaborolan-2-yl)quinazolin-4-yl)piperazine-1-carboxylate (245 mg, 0.556 mmol), Pd(dppf)Cl₂ (39 mg, 0.053 mmol) and cesium carbonate (519 mg, 1.593 mmol), along with dioxane/water mixed solvent (5 mL, v/v=5:1), were added to the reaction flask. After the addition was completed, in the presense of protective nitrogen, the reaction mixture was heated to reflux, stirred and reacted for 2 h. Upon completion of the reaction, the reaction was quenched by adding water (30 mL). The reaction mixture was extracted with ethyl acetate (60 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=2/1). Tert-butyl 4-(6-(5-((2-chloro-4-fluorophenyl)sulfonamido)-6-methoxypyridin-3-yl)pyrido[3,2-d]pyrimidin-4-yl)piperazine-1-carboxylate was obtained, yield: 65.9%; ESI-MS(m/z): 629.0 [M+H]⁺.

### Step c): Preparation of 2-chloro-4-fluoro-N-(2-methoxy-5-(4-(piperazin-1-yl)quinazolin-6-yl)pyridin-3-yl)benzenesulfonamide trifluoroacetate

Tert-butyl 4-(6-(5-((2-chloro-4-fluorophenyl)sulfonamido)-6-methoxypyridin-3-yl)pyrido[3,2-d]pyrimidin-4-yl)piperazine-1-carboxylate (220 mg, 0.350 mmol) was dissolved in dichloromethane (2 mL). Under the ice bath condition, trifluoroacetic acid (0.5 mL) was slowly added dropwise. After the addition was completed, the reaction mixture was stirred at room temperature for 1 h. Upon completion of the reaction, the reaction mixture was concentrated under reduced pressure. 2-chloro-4-fluoro-N-(2-methoxy-5-(4-(piperazin-1-yl)quinazolin-6-yl)pyridin-3-yl)benzenesulfonamide trifluoroacetate was obtained, yield: 94.7%; ESI-MS(m/z): 529.1 [M+H]⁺.

### Step d): Preparation of (L)-2-chloro-4-fluoro-N-(2-methoxy-5-(4-(4-(4-oxopent-2-enoyl)piperazin-1-yl)quinazolin-6-yl)pyridin-3-yl)benzenesulfonamide

2-chloro-4-fluoro-N-(2-methoxy-5-(4-(piperazin-1-yl)quinazolin-6-yl)pyridin-3-yl)benzenesulfonamide trifluoroacetate (90 mg, 0.140 mmol), (E)-4-oxopent-2-enoic acid (19 mg, 0.168 mmol) and tetrahydrofuran (2 mL) were added to the reaction flask. The reaction system was cooled to −78° C., with DIPEA (145 mg, 1.120 mmol) and 50% T₃P ethyl acetate solution (178 mg, 0.28 mmol, 50% wt) added successively. After the addition was completed, the reaction mixture was kept at −78° C. and stirred for 1 h. Upon completion of the reaction, at −78° C., the reaction was quenched by adding water (0 mL). The system was concentrated under reduced pressure at room temperature, diluted by adding acetonitrile (2 mL) and purified by Prep-HPLC (Method 2). (E)-2-chloro-4-fluoro-N-(2-methoxy-5-(4-(4-(4-oxopent-2-enoyl)piperazin-1-yl)quinazolin-6-yl)pyridin-3-yl)benzenesulfonamide was obtained, yield: 39.7% ¹H NMR (400 MHz, DMSO-d₅) δ 10.23 (s, 1H), 8.67 (s, 1H), 8.46-8.42 (m, 1H), 8.12-8.05 (m, 2H), 7.98-7.90 (m, 3H), 7.76-7.71 (m, 1H), 7.46 (d, J=16.0 Hz, 1H), 7.39-7.33 (m, 1H), 6.75 (d, J=16.0 Hz, 1H), 3.94-3.86 (m, 6H), 3.83-3.78 (m, 2H), 3.70 (s, 3H), 2.37 (s, 3H); ESI-MS(m/z): 625.0 [M+H]⁺.

Referring to the preparation method of Example 169 and using the corresponding raw materials, the compounds in the following examples were obtained.

| Number | Name | Structure | ¹H NMR and MS |
|---|---|---|---|
| Example 170 | (E)-N-(2- methoxy-5-(4- (4-(4-oxopent- 2-enoyl) piperazin-1-yl) quinazolin-6- yl)pyridin-3- yl)methane- sulfonamide | 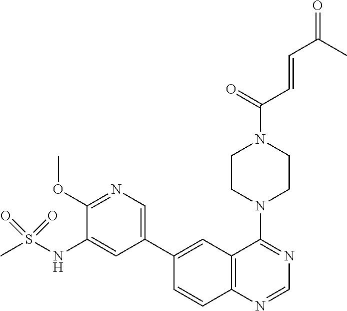 [SMILES: COc1ncc(-c2ccc3ncnc(N4CCN(C(=O)/C=C/C(C)=O)CC4)c3c2)cc1NS(C)(=O)=O] | ¹H NMR (400 MHz, DMSO-d₆) δ 9.41 (s, 1H), 8.67 (s, 1H), 8.47 (s, 1H), 8.15-8.13 (m, 2H), 8.01 (s, 1H), 7.93 (d, J = 8.8 Hz, 1H), 7.45 (d, J = 15.6 Hz, 1H), 6.73 (d, J = 15.6 Hz, 1H), 3.99 (s, 3H), 3.89-3.80 (m, 6H), 3.79-3.75 (m, 2H), 3.12 (s, 3H), 2.37 (s, 3H); ESI-MS (m/z): 511.0 [M + H]⁺. |
| Example 171 | (E)-2-chloro-4- fluoro-N-(2- methoxy-5-(4- (2-(4-oxopent- 2-enoyl)-2,7- diazaspiro[3.5] nonan-7-yl) quinazolin-6- yl)pyridin-3- yl)benzene- sulfonamide | 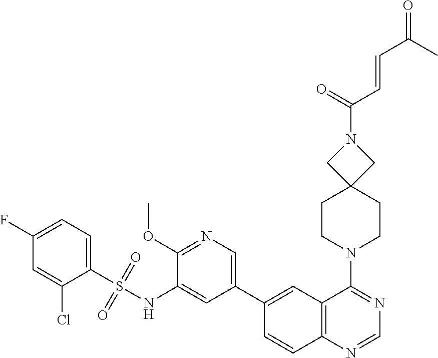 [SMILES: COc1ncc(-c2ccc3ncnc(N4CCC5(CC4)CN(C(=O)/C=C/C(C)=O)C5)c3c2)cc1NS(=O)(=O)c1ccc(F)cc1Cl] | ¹H NMR (400 MHz, DMSO-d₆) δ 10.17 (s, 1H), 8.63 (s, 1H), 8.41-8.37 (m, 1H), 8.07-8.01 (m, 2H), 7.99- 7.92 (m, 2H), 7.89 (d, J = 8.8 Hz, 1H), 7.76-7.71 (m, 1H), 7.39-7.33 (m, 1H), 6.93 (d, J = 16.0 Hz, 1H), 6.75 (d, J = 16.0 Hz, 1H), 4.15 (s, 2H), 3.80 (s, 2H), 3.77-3.73 (m, 4H), 3.71 (s, 3H), 2.35 (s, 3H), 1.96 (t, J = 5.6 Hz, 4H); ESI-MS (m/z): 665.0 [M + H]⁺. |
| Example 172 | (E)-N-(2- methoxy-5-(4- (4-(4-oxopent- 2-enoyl) piperazin-1- yl)quinazolin- 6-yl)pyridin-3- yl)-3-methyl- pyridine-4- sulfonamide | 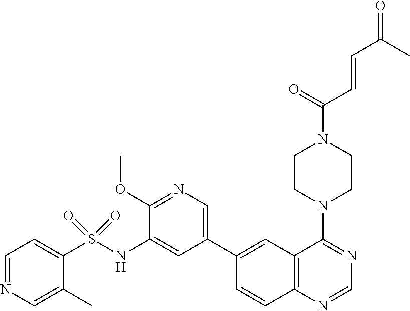 [SMILES: COc1ncc(-c2ccc3ncnc(N4CCN(C(=O)/C=C/C(C)=O)CC4)c3c2)cc1NS(=O)(=O)c1ccncc1C] | ¹H NMR (400 MHz, DMSO-d₆) δ 10.46 (s, 1H), 8.71-8.68 (m, 2H), 8.58 (d, J = 4.0 Hz, 1H), 8.48 (s, 1H), 8.12-8.07 (m, 2H), 8.00 (s, 1H), 7.92 (d, J = 8.0 Hz, 1H), 7.56 (d, J = 8.0 Hz, 1H), 7.47 (d, J = 16.0 Hz, 1H), 6.74 (d, J = 16.0 Hz, 1H), 3.92- 3.79 (m, 8H), 3.62 (s, 3H), 3.63 (s, 3H), 2.38 (s, 3H); ESI-MS (m/z): 588.0 [M + H]⁺. |
| Example 173 | (S,E)-N-(2- methoxy-5-(4- (2-methyl-4- (4-oxopent-2- enoyl) piperazin-1-yl) quinazolin-6- yl)pyridin-3- yl)-2,4- dimethyl- thiazole-5- sulfonamide | 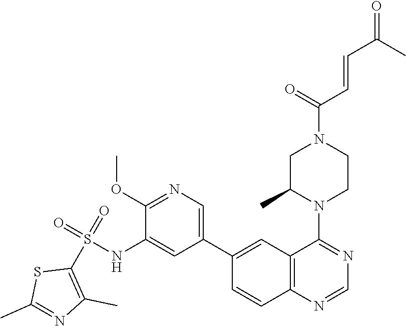 [SMILES: COc1ncc(-c2ccc3ncnc(N4CCN(C(=O)/C=C/C(C)=O)C[C@@H]4C)c3c2)cc1NS(=O)(=O)c1sc(C)nc1C] | ¹H NMR (400 MHz, DMSO-d₆) δ 10.40 (s, 1H), 8.68 (s, 1H), 8.51 (d, J = 4.0 Hz, 1H), 8.12-8.04 (m, 3H), 7.94 (d, J = 12.0 Hz, 1H), 7.45 (dd, J₁ = 40.0 Hz, J₂ = 12.0 Hz, 1H), 6.76 (dd, J₁ = 16.0 Hz, J₂ = 8.0 Hz, 1H), 4.82-4.76 (m, 1H), 4.44-4.19 (m, 3H), 3.74-3.49 (m, 5H), 3.24-3.14 (m, 1H), 2.60 (s, 3H), 2.38 (s, 6H), 1.36-1.32 (s, 3H); ESI-MS (m/z): 622.0 [M + H]⁺. |
| Example 174 | (S,E)-2- chloro-4- fluoro-N-(2- methoxy-5-(4- (2-methyl-4- (4-oxopent-2- enoyl) piperazin-1-yl) quinazolin-6- yl)pyridin-3- yl)benzene- sulfonamide | 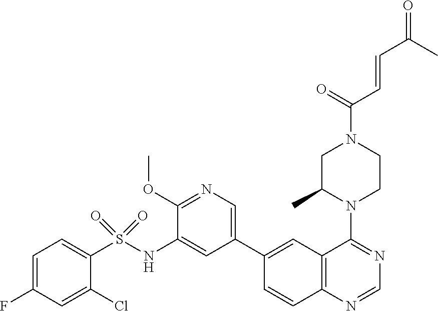 [SMILES: COc1ncc(-c2ccc3ncnc(N4CCN(C(=O)/C=C/C(C)=O)C[C@@H]4C)c3c2)cc1NS(=O)(=O)c1ccc(F)cc1Cl] | ¹H NMR (400 MHz, DMSO-d₆) δ 10.11 (s, 1H), 8.66 (s, 1H), 8.45-8.41 (m, 1H), 8.09-8.02 (m, 2H), 7.98- 7.89 (m, 3H), 7.73 (dd, J₁ = 8.8 Hz, J₂ = 2.4 Hz, 1H), 7.53-7.32 (m, 2H), 6.80-6.71 (m, 1H), 4.83-4.71 (m, 1H), 4.44-4.25 (m, 1H), 4.24-3.95 (m, 2H), 3.69 (s, 3H), 3.66-3.59 (m, 1H), 3.58-3.45 (m, 1H), 3.23-3.10 (m, 1H), 2.37 (d, J = 2.4 Hz, 3H), 1.32 (t, J = 6.8 Hz, 3H); ESI-MS (m/z): 639.0 [M + H]⁺. |
| Example 175 | 2-chloro-4- fluoro-N-(2- methoxy-5-(4- (8-((E)-4- oxopent-2- enoyl)-3,8- diazabicyclo [3.2.1]octan-3- yl)quinazolin- 6-yl)pyridin-3- yl)benzene- sulfonamide | 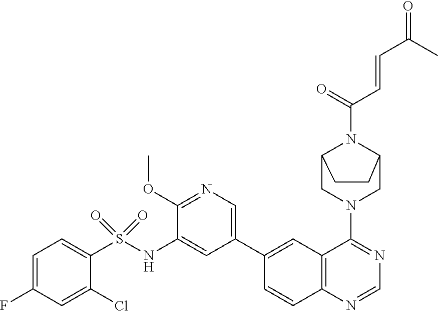 [SMILES: COc1ncc(-c2ccc3ncnc(N4CC5CCC(C4)N5C(=O)/C=C/C(C)=O)c3c2)cc1NS(=O)(=O)c1ccc(F)cc1Cl] | ¹H NMR (400 MHz, DMSO-d₆) δ 10.13 (s, 1H), 8.60 (s, 1H), 8.43-8.41 (m, 1H), 8.07-8.00 (m, 2H), 7.97- 7.92 (m, 2H), 7.87 (d, J = 8.4 Hz, 1H), 7.73 (dd, J₁ = 8.4 Hz, J₂ = 2.4 Hz, 1H), 7.43-7.38 (m, 1H), 7.38- 7.32 (m, 1H), 6.84 (d, J = 15.6 Hz, 1H), 4.80-4.72 (m, 2H), 4.55-4.44 (m, 2H), 3.69 (s, 3H), 3.59 (t, J = 11.2 Hz, 2H), 2.39 (s, 3H), 1.98-1.89 (m, 1H), 1.85-1.74 (m, 3H); ESI-MS (m/z): 651.0 [M + H]⁺. |
| Example 176 | N-(2-methoxy- 5-(4-(8-((E)-4- oxopent-2- enoyl)-3,8- diazabicyclo [3.2.1]octan-3- yl)quinazolin- 6-yl)pyridin-3- yl)-2,4- dimethyl- thiazole-5- sulfonamide | 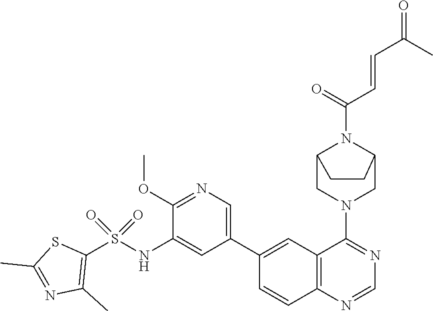 [SMILES: COc1ncc(-c2ccc3ncnc(N4CC5CCC(C4)N5C(=O)/C=C/C(C)=O)c3c2)cc1NS(=O)(=O)c1sc(C)nc1C] | ¹H NMR (400 MHz, DMSO-d₆) δ 10.36 (s, 1H), 8.61 (s, 1H), 8.48 (s, 1H), 8.10-8.04 (m, 2H), 8.00 (s, 1H), 7.90 (d, J = 8.4 Hz, 1H), 7.42 (d, J = 15.6 Hz, 1H), 6.84 (d, J = 15.6 Hz, 1H), 4.80-4.75 (m, 2H), 4.56-4.46 (m, 2H), 3.74 (s, 3H), 3.61 (t, J = 13.2 Hz, 2H), 2.59 (s, 3H), 2.40- 2.38 (m, 6H), 1.95-1.92 (m, 1H), 1.85-1.81 (m, 3H); ESI-MS (m/z): 634.0 [M + H]⁺. |
| Example 177 | (E)-4-chloro-2- fluoro-N-(2- methoxy-5-(4- (4-(4-oxopent- 2-enoyl) piperazin-1- yl)quinazolin- 6-yl)pyridin-3- yl)benzene- sulfonamide | 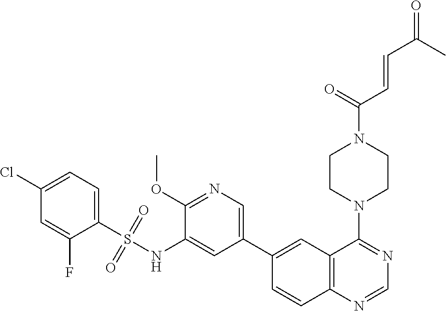 [SMILES: COc1ncc(-c2ccc3ncnc(N4CCN(C(=O)/C=C/C(C)=O)CC4)c3c2)cc1NS(=O)(=O)c1ccc(Cl)cc1F] | ¹H NMR (400 MHz, DMSO-d₆) δ 10.39 (s, 1H), 8.66 (s, 1H), 8.46-8.44 (m, 1H), 8.13-8.07 (m, 2H), 8.01- 7.99 (m, 1H), 7.91 (d, J = 8.8 Hz, 1H), 7.77-7.69 (m, 2H), 7.47-7.40 (m, 2H), 6.74 (d, J = 16.0 Hz, 1H), 3.92-3.87 (m, 6H), 3.83-3.78 (m, 2H), 3.67 (s, 3H), 2.37 (s, 3H); ESI- MS (m/z): 625.0 [M + H]⁺. |
| Example 178 | (E)-N-(2- methoxy-5-(4- (2-(4-oxopent- 2-enoyl)-2,7- diazaspiro[3.5] nonan-7-yl) quinazolin-6- yl)pyridin-3- yl)-2,4- dimethyl- thiazole-5- sulfonamide | 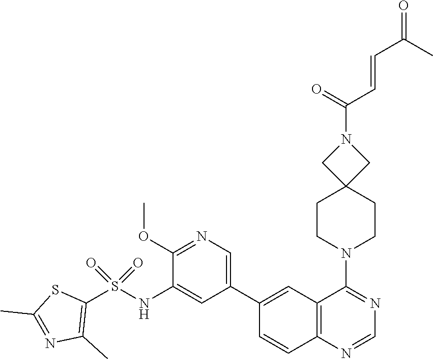 [SMILES: COc1ncc(-c2ccc3ncnc(N4CCC5(CC4)CN(C(=O)/C=C/C(C)=O)C5)c3c2)cc1NS(=O)(=O)c1sc(C)nc1C] | ¹H NMR (400 MHz, DMSO-d₆) δ 10.31 (s, 1H), 8.64 (d, J = 4.0 Hz, 1H), 8.42 (s, 1H), 8.08-7.89 (m, 4H), 6.96-6.91 (m, 1H), 6.77-6.72 (m, 1H), 4.15 (s, 2H), 3.08-3.72 (m, 9H), 2.59 (s, 3H), 2.38-2.34 (m, 6H), 1.99-1.96 (m, 4H); ESI-MS (m/z): 648.0 [M + H]⁺. |
| Example 179 | (E)-4-(4-(6-(5- ((2,4- difluorophenyl) sulfonamido)- 6-methoxy- pyridin-3-yl) quinazolin-4- yl)piperazin-1- yl)-N-methyl- 4-oxobut-2- enamide | 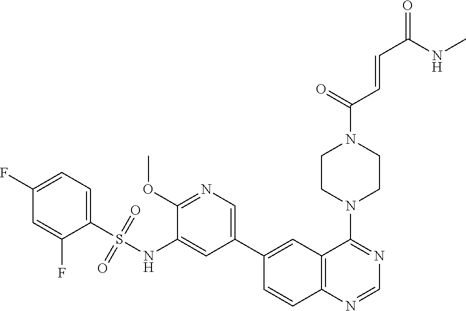 [SMILES: CNC(=O)/C=C/C(=O)N1CCN(c2ncnc3ccc(-c4cnc(OC)c(NS(=O)(=O)c5ccc(F)cc5F)c4)cc23)CC1] | ¹H NMR (400 MHz, DMSO-d₆) δ 8.65 (s, 1H), 8.29 (s, 1H), 8.16-8.13 (m, 1H), 8.05-8.02 (m, 2H), 7.91- 7.89 (m, 2H), 7.82-7.76 (m, 1H), 7.51-7.45 (m, 1H), 7.21-7.16 (m, 1H), 6.42 (d, J = 11.6 Hz, 1H), 6.05 (d, J = 11.6 Hz, 1H), 3.85-3.81 (m, 4H), 3.74-3.72 (m, 5H), 3.56-3.54 (m, 2H), 2.62 (d, J = 4.8 Hz, 3H); ESI-MS (m/z): 624.0 [M + H]⁺. |
| Example 180 | (E)-N-(5- (4-(4-(2-cyano- 4,4-dimethyl- pent-2-enoyl) piperazin-1- yl)quinazolin- 6-yl)-2- methoxy- pyridin-3-yl)- 2,4-difluoro- benzene- sulfonamide | 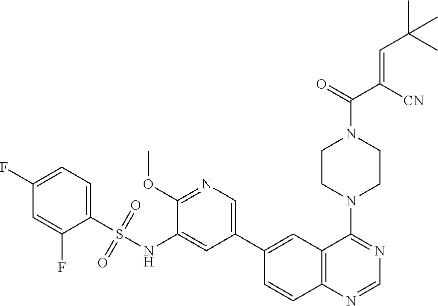 [SMILES: COc1ncc(-c2ccc3ncnc(N4CCN(C(=O)/C(C#N)=C/C(C)(C)C)CC4)c3c2)cc1NS(=O)(=O)c1ccc(F)cc1F] | ¹H NMR (400 MHz, DMSO-d₆) δ 10.46 (s, 1H), 8.66 (s, 1H), 8.32 (s, 1H), 8.14-8.04 (m, 2H), 7.93-7.90 (m, 2H), 7.81-7.75 (m, 1H), 7.48 (t, J = 10.0 Hz, 1H), 7.20-7.15 (m, 1H), 6.91 (s, 1H), 3.90-3.88 (m, 4H), 3.77-3.70 (m, 7H), 1.24 (s, 9H); ESI-MS (m/z): 648.0 [M + H]⁺. |
| Example 181 | methyl (E)-3- ((2,4-difluoro- phenyl) sulfonamido)- 5-(4-(4-(4- oxopent-2- enoyl) piperazin-1-yl) quinazolin-6- yl)picolinate | 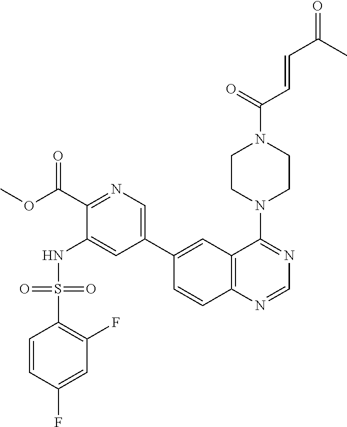 [SMILES: COC(=O)c1ncc(-c2ccc3ncnc(N4CCN(C(=O)/C=C/C(C)=O)CC4)c3c2)cc1NS(=O)(=O)c1ccc(F)cc1F] | ¹H NMR (400 MHz, DMSO-d₆) δ 10.83 (br s, 1H), 8.83 (s, 1H), 8.69 (s, 1H), 8.27-8.08 (m, 3H), 8.00-7.89 (m, 2H), 7.58-7.48 (m, 1H), 7.45 (d, J = 16.0 Hz, 1H), 7.28-7.19 (m, 1H), 6.74 (d, J = 15.6 Hz, 1H), 4.10-3.79 (m, 8H), 3.79 (s, 3H), 2.36 (s, 3H); ESI-MS (m/z): 637.0 [M + H]⁺. |
| Example 182 | (E)-2,4- difluoro-N-(2- (methylthio)- 5-(4-(4-(4- oxopent-2- enoyl) piperazin-1-yl) quinazolin-6- yl)pyridin-3- yl)benzene- sulfonamide | 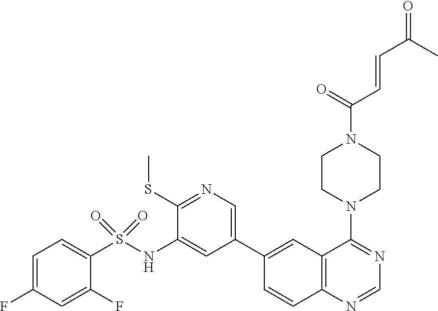 [SMILES: CSc1ncc(-c2ccc3ncnc(N4CCN(C(=O)/C=C/C(C)=O)CC4)c3c2)cc1NS(=O)(=O)c1ccc(F)cc1F] | ¹H NMR (400 MHz, DMSO-d₆) δ 10.61 (s, 1H), 8.65 (s, 1H), 8.32 (s, 1H), 8.03 (s, 1H), 7.97-7.94 (m, 1H), 7.89 (d, J = 8.4 Hz, 1H), 7.82-7.76 (m, 1H), 7.62 (s, 1H), 7.45 (d, J = 16.0 Hz, 1H), 7.28 (s, 1H), 7.11-7.06 (m, 1H), 6.73 (d, J = 16.0 Hz, 1H), 3.89-3.79 (m, 8H), 2.37 (s, 3H), 2.35 (s, 3H); ESI-MS (m/z): 625.0 [M + H]⁺. |
| Example 183 | (E)-2,4- difluoro-N-(2- (hydroxy- methyl)-5-(4- (4-(4-oxopent- 2-enoyl) piperazin-1- yl)quinazolin- 6-yl)pyridin-3- yl)benzene- sulfonamide | 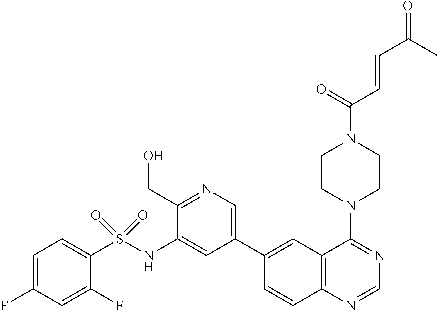 [SMILES: CC(=O)/C=C/C(=O)N1CCN(c2ncnc3ccc(-c4cnc(CO)c(NS(=O)(=O)c5ccc(F)cc5F)c4)cc23)CC1] | ¹H NMR (400 MHz, DMSO-d₆) δ 8.66 (s, 1H), 8.10 (d, J = 2.0 Hz, 1H), 7.99 (s, 1H), 7.97-7.86 (m, 2H), 7.84-7.78 (m, 1H), 7.70 (d, J = 2.0 Hz, 1H), 7.45 (d, J = 16.0 Hz, 1H), 7.26-7.16 (m, 1H), 7.11-7.01 (m, 1H), 6.73 (d, J = 16.0 Hz, 1H), 5.29 (t, J = 5.6 Hz, 1H), 4.54 (d, J = 5.6 Hz, 2H), 4.04-3.61 (m, 8H), 2.37 (s, 3H); ESI-MS (m/z): 609.0 [M + H]⁺. |
| Example 184 | (E)-2-chloro-5- methoxy-N-(2- methoxy-5-(4- (4-(4-oxopent- 2-enoyl) piperazin-1- yl)quinazolin- 6-yl)pyridin-3- yl)benzene- sulfonamide | 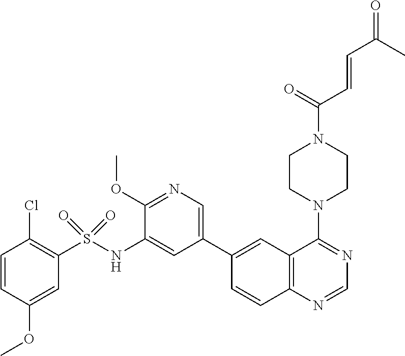 [SMILES: COc1ccc(Cl)c(S(=O)(=O)Nc2cc(-c3ccc4ncnc(N5CCN(C(=O)/C=C/C(C)=O)CC5)c4c3)cnc2OC)c1] | ¹H NMR (400 MHz, DMSO-d₆) δ 10.21 (s, 1H), 8.66 (s, 1H), 8.36 (s, 1H), 8.09-8.00 (m, 2H), 7.93-7.88 (m, 2H), 7.55 (d, J = 8.8 Hz, 1H), 7.48-7.42 (m, 2H), 7.18 (dd, J₁ = 8.8 Hz, J₂ = 3.2 Hz, 1H), 6.74 (d, J = 15.6 Hz, 1H), 3.91-3.85 (m, 6H), 3.82-3.77 (m, 2H), 3.76-3.73 (m, 6H), 2.37 (s, 3H); ESI-MS (m/z): 637.0 [M + H]⁺. |
| Example 185 | (E)-2-fluoro-4- methoxy-N-(2- methoxy-5-(4- (4-(4-oxopent- 2-enoyl) piperazin-1- yl)quinazolin- 6-yl)pyridin-3- yl)benzene- sulfonamide | 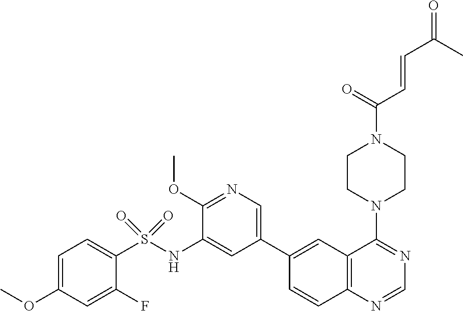 [SMILES: COc1ccc(S(=O)(=O)Nc2cc(-c3ccc4ncnc(N5CCN(C(=O)/C=C/C(C)=O)CC5)c4c3)cnc2OC)c(F)c1] | ¹H NMR (400 MHz, DMSO-d₆) δ 9.71 (s, 1H), 8.66 (s, 1H), 8.21 (s, 1H), 8.06-7.99 (m, 2H), 7.90 (d, J = 8.8 Hz, 1H), 7.84 (s, 1H), 7.64 (t, J = 8.8 Hz, 1H), 7.45 (d, J = 16.0 Hz, 1H), 7.00-6.93 (m, 1H), 6.84-6.79 (m, 1H), 6.74 (d, J = 16.0 Hz, 1H), 3.90-3.84 (m, 6H), 3.82-3.77 (m, 5H), 3.74 (s, 3H), 2.37 (s, 3H); ESI-MS (m/z): 621.0 [M + H]⁺. |
| Example 186 | (E)-2-fluoro- N-(2-methoxy- 5-(4-(4-(4- oxopent-2- enoyl) piperazin-1-yl) quinazolin-6- yl)pyridin-3- yl)-6-methyl- benzene- sulfonamide | 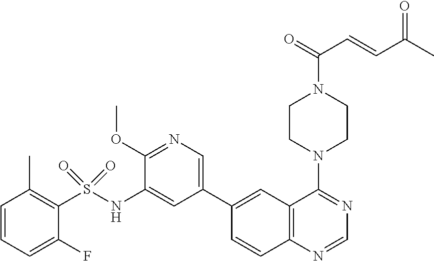 [SMILES: COc1ncc(-c2ccc3ncnc(N4CCN(C(=O)/C=C/C(C)=O)CC4)c3c2)cc1NS(=O)(=O)c1c(C)cccc1F] | ¹H NMR (400 MHz, DMSO-d₆) δ 10.10 (br s, 1H), 8.67 (s, 1H), 8.48- 8.38 (m, 1H), 8.11 (d, J = 2.0 Hz, 1H), 8.06 (d, J = 8.8 Hz, 1H), 7.99 (d, J = 2.4 Hz, 1H), 7.92 (d, J = 8.8 Hz, 1H), 7.57-7.40 (m, 2H), 7.27- 7.13 (m, 2H), 6.74 (d, J = 15.6 Hz, 1H), 3.96-3.75 (m, 8H), 3.64 (s, 3H), 2.49 (s, 3H), 2.37 (s, 3H); ESI- MS (m/z): 605.0 [M + H]⁺. |
| Example 187 | (E)-4-fluoro- N-(2- methoxy-5-(4- (4-(4-oxopent- 2-enoyl) piperazin-1- yl)quinazolin- 6-yl)pyridin-3- yl)naphthalene- 1-sulfonamide | 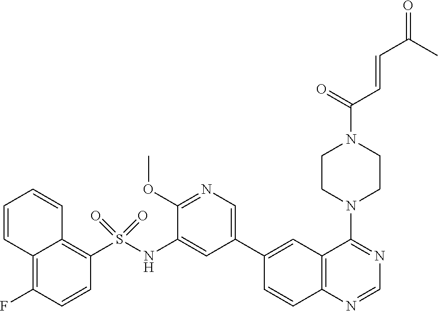 [SMILES: COc1ncc(-c2ccc3ncnc(N4CCN(C(=O)/C=C/C(C)=O)CC4)c3c2)cc1NS(=O)(=O)c1ccc(F)c2ccccc12] | ¹H NMR (400 MHz, DMSO-d₆) δ 10.35 (br s, 1H), 8.81 (d, J = 8.4 Hz, 1H), 8.66 (s, 1H), 8.39-8.32 (m, 1H), 8.23-8.17 (m, 1H), 8.12-8.01 (m, 3H), 7.97-7.89 (m, 2H), 7.88-7.76 (m, 2H), 7.49-7.37 (m, 2H), 6.74 (d, J = 15.6 Hz, 1H), 3.92-3.75 (m, 8H), 3.28 (s, 3H), 2.37 (s, 3H); ESI-MS (m/z): 641.0 [M + H]⁺. |
| Example 188 | (E)-2,4- difluoro-N-(2- methyl-5-(4- (4-(4-oxopent- 2-enoyl) piperazin-1-yl) quinazolin-6- yl)pyridin-3- yl)benzene- sulfonamide | 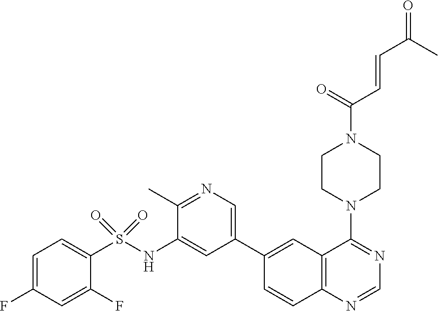 [SMILES: CC(=O)/C=C/C(=O)N1CCN(c2ncnc3ccc(-c4cnc(C)c(NS(=O)(=O)c5ccc(F)cc5F)c4)cc23)CC1] | ¹H NMR (400 MHz, DMSO-d₆) δ 10.58 (s, 1H), 8.67 (s, 1H), 8.65 (s, 1H), 8.12-8.10 (m, 1H), 8.05-8.01 (m, 1H), 7.92 (d, J = 8.8 Hz, 1H), 7.85-7.77 (m, 2H), 7.56-7.49 (m, 1H), 7.45 (d, J = 16.0 Hz, 1H), 7.24- 7.17 (m, 1H), 6.74 (d, J = 15.6 Hz, 1H), 3.92-3.86 (m, 6H), 3.82-3.77 (m, 2H), 2.37 (s, 3H), 2.32 (s, 3H); ESI-MS (m/z): 593.0 [M + H]⁺. |
| Example 189 | (E)-N-(2- cyano-5-(4-(4- (4-oxopent-2- enoyl) piperazin-1-yl) quinazolin-6- yl)pyridin-3- yl)-2,4- difluoro- benzene- sulfonamide | 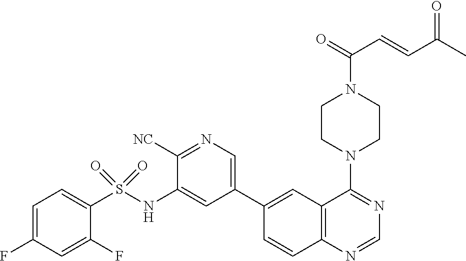 [SMILES: CC(=O)/C=C/C(=O)N1CCN(c2ncnc3ccc(-c4cnc(C#N)c(NS(=O)(=O)c5ccc(F)cc5F)c4)cc23)CC1] | ¹H NMR (400 MHz, DMSO-d₆) δ 8.79 (s, 1H), 8.55 (br s, 1H), 8.25 (d, J = 2.0 Hz, 1H), 8.11 (d, J = 8.4 Hz, 1H), 8.05 (d, J = 2.0 Hz, 1H), 7.94 (d, J = 8.8 Hz, 1H), 7.91-7.83 (m, 1H), 7.47-7.33 (m, 2H), 7.22-7.11 (m, 1H), 6.75 (d, J = 15.6 Hz, 1H), 4.18-4.03 (m, 4H), 3.97-3.86 (m, 2H), 3.84-3.77 (m, 2H), 2.37 (s, 3H); ESI-MS (m/z): 604.0 [M + H]⁺. |
| Example 190 | (E)-N-(2- ethoxy-5-(4-(4- (4-oxopent-2- enoyl) piperazin-1-yl) quinazolin-6- yl)pyridin-3- yl)-2-fluoro- benzene- sulfonamide | 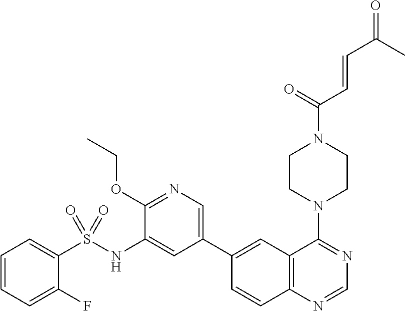 [SMILES: CCOc1ncc(-c2ccc3ncnc(N4CCN(C(=O)/C=C/C(C)=O)CC4)c3c2)cc1NS(=O)(=O)c1ccccc1F] | ¹H NMR (400 MHz, DMSO-d₆) δ 8.66 (s, 1H), 8.27 (s, 1H), 8.11-7.97 (m, 2H), 7.90 (d, J = 8.8 Hz, 2H), 7.78-7.70 (m, 1H), 7.67-7.56 (m, 1H), 7.45 (d, J = 15.6 Hz, 1H), 7.41- 7.34 (m, 1H), 7.33-7.19 (m, 1H), 6.74 (d, J = 15.6 Hz, 1H), 4.17 (dd, J₁ = 14.0 Hz, J₂ = 7.2 Hz, 2H), 4.03- 3.60 (m, 8H), 2.37 (s, 3H), 1.15 (d, J = 7.2 Hz, 3H); ESI-MS (m/z): 605.0 [M + H]⁺. |
| Example 191 | (E)-2,4- difluoro-N-(2- (methoxy-d₃)- 5-(4-(4-(4- oxopent-2- enoyl) piperazin-1-yl) quinazolin-6- yl)pyridin-3- yl)benzene- sulfonamide | 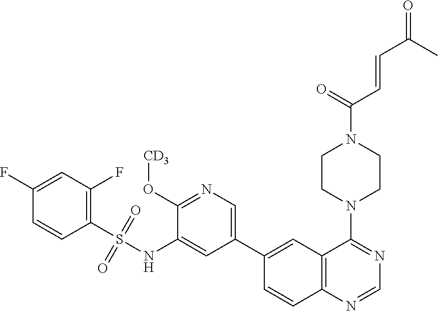 [SMILES: [2H]C([2H])([2H])Oc1ncc(-c2ccc3ncnc(N4CCN(C(=O)/C=C/C(C)=O)CC4)c3c2)cc1NS(=O)(=O)c1ccc(F)cc1F] | ¹H NMR (400 MHz, DMSO-d₆) δ 10.28 (s, 1H), 8.66 (s, 1H), 8.39 (s, 1H), 8.17-8.02 (m, 2H), 8.00-7.85 (m, 2H), 7.82-7.72 (m, 1H), 7.60- 7.48 (m, 1H), 7.45 (d, J = 15.6 Hz, 1H), 7.25-7.12 (m, 1H), 6.74 (d, J = 15.6 Hz, 1H), 4.01-3.68 (m, 8H), 2.37 (s, 3H); ESI-MS (m/z): 612.0 [M + H]⁺. |
| Example 192 | (E)-2,4- difluoro-N-(5- (4-(4-(4- oxopent-2- enoyl) piperazin-1- yl)quinazolin- 6-yl)-2-(tri- fluoromethyl) pyridin-3-yl) benzene- sulfonamide | 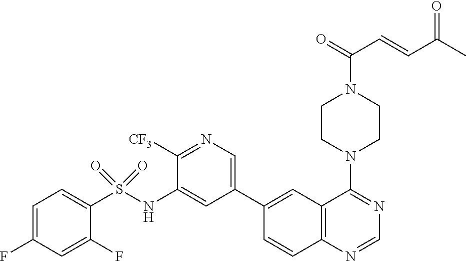 [SMILES: CC(=O)/C=C/C(=O)N1CCN(c2ncnc3ccc(-c4cnc(C(F)(F)F)c(NS(=O)(=O)c5ccc(F)cc5F)c4)cc23)CC1] | ¹H NMR (400 MHz, DMSO-d₆) δ 8.87 (br s, 1H), 8.77 (s, 1H), 8.32 (s, 1H), 8.22-8.15 (m, 2H), 7.97 (d, J = 8.4 Hz, 1H), 7.88-7.79 (m, 1H), 7.53-7.38 (m, 2H), 7.24-7.17 (m, 1H), 6.75 (d, J = 15.6 Hz, 1H), 4.13- 4.01 (m, 4H), 3.96-3.88 (m, 2H), 3.85-3.78 (m, 2H), 2.37 (s, 3H); ESI- MS (m/z): 647.0 [M + H]⁺. |

### Example 19

### Preparation of N-(5-(4-((2R,6S)-2,6-dimethyl-4-((E)-4-oxopent-2-enoyl)piperazin-1-yl)quinazolin-6-yl)-2-methoxypyridin-3-yl)-2-fluorobenzenesulfonamide

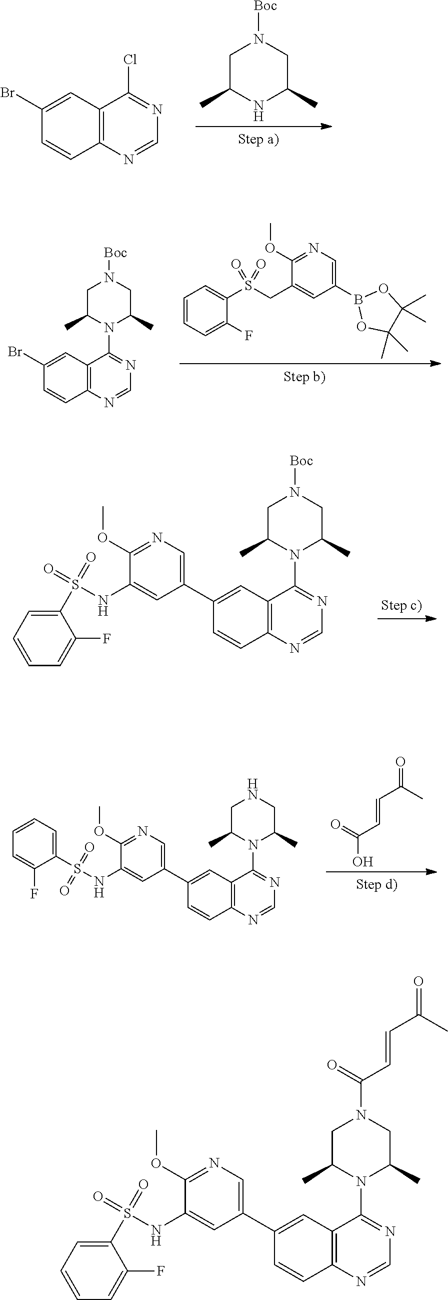

### Step a): Preparation of tert-butyl (3R,5S)-4-(6-bromoquinazolin-4-yl)-3,5-dimethylpiperazine-1-carboxylate

6-Bromo-4-chloroquinazoline (500 mg, 2.053 mmol), tert-butyl (3R,5S)-3,5-dimethylpiperazine-1-carboxylate (880 mg, 4.106 mmol), sodium bicarbonate (517 mg, 6.159 mmol) and acetonitrile (20 mL) were added to the reaction flask. After the addition was completed, the reaction mixture was microwave heated to 110° C. and reacted for 8 h. Upon completion of the reaction, the reaction was quenched by adding water (50 mL). The reaction mixture was extracted with ethyl acetate (100 mL×2). The organic phases were combined, washed with saturated brine (50 mL), dried over anhydrous sodium sulfate and filtered. The filtrate was concentrated under reduced pressure. The crude product obtained was purified by silica gel column chromatography (Eluent: dichloromethane/methanol=100/1 to 20/1). Tert-butyl (3R,5S)-4-(6-bromoquinazolin-4-yl)-3,5-dimethylpiperazine-1-carboxylate was obtained, yield: 76.9%; ESI-MS(m/z): 421.1 [M+H]⁺.

### Step b): Preparation of tert-butyl (3R,5S)-4-(6-(5-((2-fluorophenyl)sulfonamido)-6-methoxypyridin-3-yl)quinazolin-4-yl)-3,5-dimethylpiperazine-1-carboxylate

Tert-butyl (3R,5S)-4-(6-bromoquinazolin-4-yl)-3,5-dimethylpiperazine-1-carboxylate (200 mg, 0.475 mmol), 2-fluoro-N-(2-methoxy-5-(4,4,5,5-tetramethyl-1,3,2-dioxaborolan-2-yl)pyridin-3-yl)benzenesulfonamide (194 mg, 0.475 mmol), Pd(dppf)Cl₂ (35 mg, 0.048 mmol) and cesium carbonate (464 mg, 1.425 mmol), dioxane/water mixed solvent (10 mL, v/v=10:1) were added to the reaction flask. After the addition was completed, in the presense of protective nitrogen, the reaction mixture was heated to 100° C. and reacted for 2 h. Upon completion of the reaction, the reaction was quenched by adding water (50 mL). The reaction mixture was extracted with ethyl acetate (100 mL×2). The organic phases were combined, washed with saturated brine (50 mL), dried over anhydrous sodium sulfate and filtered. The filtrate was concentrated under reduced pressure. The crude product obtained was purified by silica gel column chromatography (Eluent: dichloromethane/methanol=100/1 to 50/1). Tert-butyl (3R,5S)-4-(6-(5-((2-fluorophenyl)sulfonamido)-6-methoxypyridin-3-yl)quinazolin-4-yl)-3,5-dimethylpiperazine-1-carboxylate was obtained, yield: 64.5%; ESI-MS(m/z): 623.2 [M+H]⁺.

### Step c): Preparation of N-(5-(4-((2R,6S)-2,6-dimethylpiperazin-1-yl)quinazolin-6-yl)-2-methoxypyridin-3-yl)-2-fluorobenzenesulfonamide trifluoroacetate

Tert-butyl (3R,5S)-4-(6-(5-((2-fluorophenyl)sulfonamido)-6-methoxypyridin-3-yl)quinazolin-4-yl)-3,5-dimethylpiperazine-1-carboxylate (100 mg, 0.161 mmol), dichloromethane (4 mL) were added to the reaction flask, under the ice bath condition. TFA (1 mL) was slowly added dropwise. After the addition was completed, the reaction mixture was stirred at room temperature for 1 h. Upon completion of the reaction, the reaction mixture was concentrated under reduced pressure. The crude product was slurried with methyl tert-butyl ether (5 mL) to precipitate solid and filtered. N-(5-(4-((2R,6S)-2,6-dimethylpiperazin-1-yl)quinazolin-6-yl)-2-methoxypyridin-3-yl)-2-fluorobenzenesulfonamide trifluoroacetate was obtained, yield: 90.4%; ESI-MS(m/z): 523.2 [M+H]⁺.

### Step d): Preparation of N-(5-(4-((2R,6S)-2,6-dimethyl-4-((E)-4-oxopent-2-enoyl)piperazin-1-yl)quinazolin-6-yl)-2-methoxypyridin-3-yl)-2-fluorobenzenesulfonamide

N-(5-(4-((2R,6S)-2,6-dimethylpiperazin-1-yl)quinazolin-6-yl)-2-methoxypyridin-3-yl)-2-fluorobenzenesulfonamide trifluoroacetate (60 mg, 0.094 mmol) and tetrahydrofuran (2 mL) were added to the reaction flask. The reaction system was cooled to −78° C., with DIPEA (73 mg, 0.564 mmol), (E)-4-oxopent-2-enoic acid (11 mg, 0.094 mmol) and 50% T₃P ethyl acetate solution (120 mg, 0.188 mmol, 50% wt) added successively. After the addition was completed, the reaction mixture was kept at −78° C. and stirred for 1 h. Upon completion of the reaction, the reaction solution was diluted by adding acetonitrile (1 mL) and purified by Prep-HPLC (Method 2). N-(5-(4-((2R,6S)-2,6-dimethyl-4-((E)-4-oxopent-2-enoyl)piperazin-1-yl)quinazolin-6-yl)-2-methoxypyridin-3-yl)-2-fluorobenzenesulfonamide was obtained, yield: 28.8%. ¹H NMR (400 MHz, DMSO-d₆) δ 8.74 (s, 1H), 8.16-8.13 (m, 2H), 8.07-8.01 (m, 1H), 7.92 (d, J=8.8 Hz, 1H), 7.87-7.79 (m, 1H), 7.77-7.71 (m, 1H), 7.60-7.51 (m, 1H), 7.44 (d, J=16.0 Hz, 1H), 7.32 (t, J=9.2 Hz, 1H), 7.24 (t, J=7.6 Hz, 1H), 6.81 (d, J=15.6 Hz, 1H), 4.61-4.47 (m, 2H), 3.97-3.74 (m, 3H), 3.73 (s, 3H), 3.47 (dd, J=13.2, J₂=4.0 Hz, 1H), 2.38 (s, 3H), 1.30 (d, J=6.8 Hz, 6H); ESI-MS(m/z): 619.0 [M+H]⁺.

### Example 194

### Preparation of (E)-2,4-difluoro-N-(5-(4-(4-(4-oxopent-2-enoyl)piperazin-1-yl)quinazolin-6-yl)-2-(trifluoromethoxy)pyridin-3-yl)benzenesulfonamide

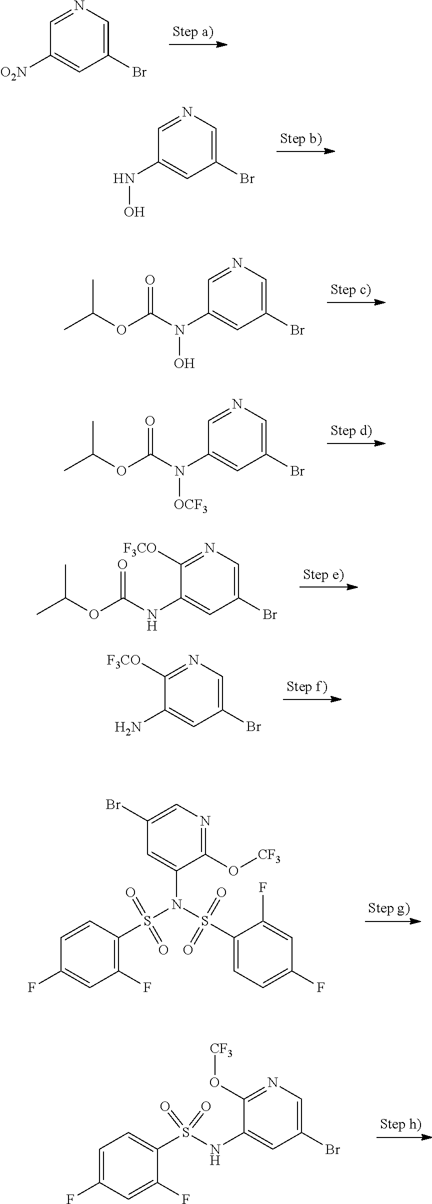

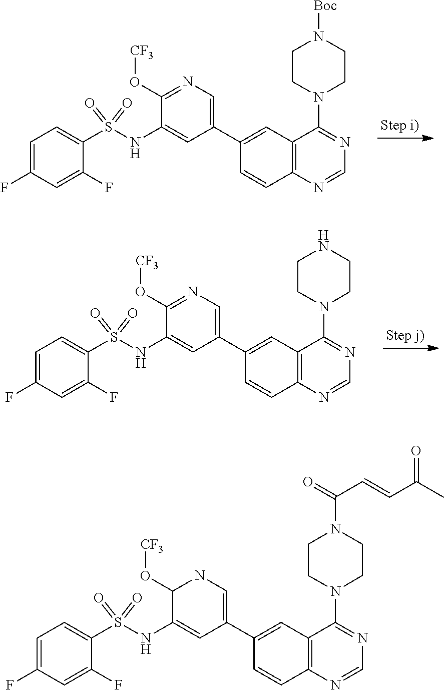 [SMILES: CC(C)(C)OC(=O)N1CCN(c2ncnc3ccc(-c4cnc(OC(F)(F)F)c(NS(=O)(=O)c5ccc(F)cc5F)c4)cc23)CC1 ; O=S(=O)(Nc1cc(-c2ccc3ncnc(N4CCNCC4)c3c2)cnc1OC(F)(F)F)c1ccc(F)cc1F ; CC(=O)/C=C/C(=O)N1CCN(c2ncnc3ccc(C4=CNC(OC(F)(F)F)C(NS(=O)(=O)c5ccc(F)cc5F)=C4)cc23)CC1]

### Step a): Preparation of N-(5-bromopyridin-3-yl)hydroxylamine

3-Bromo-5-nitropyridine (15 g, 0.074 mol) and tetrahydrofuran (150 mL) were added to the reaction flask, stirred to dissolve, with 10% Pd/C (3.93 g, water 55%). Hydrazine hydrate (6 g, 0.089 mol, 80% aqueous solution) was slowly added to the system dropwise. After the addition was completed, the reaction mixture was stirred at room temperature and reacted for 3 h. Upon completion of the reaction, the reaction mixture was filtered. The filter cake was washed with methanol (100 mL×2), the filtrate was combined, concentrated under reduced pressure. The crude product obtained was purified by silica gel column chromatography (Eluent: dichloromethane/methanol=35/1). N-(5-bromopyridin-3-yl)hydroxylamine was obtained, yield: 76.8%; ¹H NMR (400 MH z, DMSO-d₆) δ 8.81 (s, 1H), 8.73 (d, 0.1=2.0 Hz, 1H), 8.09 (d, J₁=2.0 Hz, 1H), 8.04 (d, J=2.4 Hz, 1H), 7.38-7.33 (m, 1H).

### Step b): Preparation of isopropyl (5-bromopyridin-3-yl)(hydroxy)carbamate

N-(5-bromopyridin-3-yl)hydroxylamine (3000 mg, 15.872 mmol), sodium bicarbonate (1600 mag, 19.047 mmol), 4-dimethylaminopyridine (1939 mg, 15.872 mmol) and tetrahydrofuran (70 mL) e we added to three-necked bottle. In the presense of protective nitrogen, the reaction mixture was cooled to −78° C., with isopropyl chloroformate (2139 mg, 17.459 mmol) tetrahydrofuran (2 mL) solution slowly added. After the addition was completed, the reaction mixture was slowly heated to room temperature and stirred for 18 h. Upon completion of the reaction, the reaction was quenched by adding water (50 mL). The reaction mixture was extracted with ethyl acetate (70 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=5/1 to 3/1). Isopropyl (5-bromopyridin-3-yl)(hydroxy)carbamate was obtained, yield: 28.3%; ¹H NMR (400 MHz, CDCl₃) δ 8.75 (d, J=2.4 Hz, 1H), 8.36 (d, J=2.0 Hz, 1H), 8.22 (d, J=2.0 Hz, 1H), 5.19-5.07 (m, 1H), 1.37 (d, J=6.4 Hz, 6H); ESI-MS(m/z): 274.9 [M+H]⁺.

### Step c): Preparation of isopropyl (5-bromopyridin-3-yl)(trifluoromethoxy)carbamate

Isopropyl (5-bromopyridin-3-yl)(hydroxy)carbamate (1237 mg, 4.496 mmol) was dissolved in dichloromethane (60 mL), with 3,3-dimethyl-1-(trifluoromethyl)-1,2-benzoiodooxolane (1484 mg, 4.496 mmol) added. After the addition was completed, in the presense of protective nitrogen, the reaction mixture was stirred at room temperature and reacted for 15 h. Upon completion of the reaction, the reaction mixture was concentrated under reduced pressure. The crude product obtained was purified by silica gel column chromatography (Eluent: petroleum ether/ethyl acetate=10/1). Isopropyl (5-bromopyridin-3-yl)(trifluoromethoxy)carbamate was obtained, yield: 70.0%; ¹H NMR (400 MHz, CDCl₃) δ 8.66-8.58 (m, 2H), 7.93 (d, J=2.0 Hz, 1H), 5.16-5.05 (m, 1H), 1.34 (d, J=6.4 Hz, 6H); ESI-MS(m/z): 343.1 [M+H]⁺.

### Step d): Preparation of isopropyl (5-bromo-2-(trifluoromethoxy)pyridin-3-yl)carbamate

Isopropyl (5-bromopyridin-3-yl)(trifluoromethoxy)carbamate (1350 mg, 3.935 mmol) and nitromethane (36 mL) were mixed. The reaction mixture was microwave heated to 160° C. and stirred for 4 h. Upon completion of the reaction, the reaction mixture was concentrated under reduced pressure. The crude product obtained was purified by silica gel column chromatography (Eluent: petroleum ether/ethyl acetate=10/1). Isopropyl (5-bromo-2-(trifluoromethoxy)pyridin-3-yl)carbamate was obtained, yield: 31.0%; ¹H NMR (400 MHz, CDCl₃) δ 8.76 (br s, 1H), 7.9% (d, J=2.4 Hz, 1H), 6.80 (s, 1H), 5.10-4.98 (m, 1H), 1.34 (d, J=6.4 Hz, 6H); ESI-MS(m/z): 343.1 [M+H]⁺.

### Step e): Preparation of 5-bromo-2-(trifluoromethoxy)pyridin-3-amine

Isopropyl (5-bromo-2-(trifluoromethoxy)pyridin-3-yl)carbamate (420 mg, 1.224 mmol) and methanol (15 mL) and water (2 mL) were mixed, dissolved and added to the reaction flask, with lithium hydroxide monohydrate (206 mg, 4.896 mmol) added. After the addition was completed, in the presense of protective nitrogen, the reaction mixture was kept at 60° C., stirred and reacted for 15 h. Upon completion of the reaction, the reaction mixture was extracted by adding dichloromethane (30 mL) and water (20 mL). After that, the aqueous phase was extracted with dichloromethane (20 mL×2). The organic phases were combined, dried over anhydrous sodium sulfate and filtered. The filtrate was concentrated under reduced pressure. 5-Bromo-2-(trifluoromethoxy)pyridin-3-amine was obtained, yield: 78.5%; ESI-MS(m/z): 257.0 [M+H]⁺.

### Step f): Preparation of N-(5-bromo-2-(trifluoromethoxy)pyridin-3-yl)-N-((2,4-difluorophenyl)sulfonyl)-2,4-difluorobenzenesulfonamide

5-Bromo-2-(trifluoromethoxy)pyridin-3-amine (246 mg, 0.957 mmol) was dissolved in pyridine (9 mL), with 2,4-difluorobenzenesulfonyl chloride (1017 mg, 4.786 mmol) added. After the addition was completed, at 40° C., the reaction mixture was stirred and reacted for 18 h. Upon completion of the reaction, the reaction mixture was concentrated under reduced pressure. N-(5-bromo-2-(trifluoromethoxy)pyridin-3-yl)-N-((2,4-difluorophenyl)sulfonyl)-2,4-difluorobenzenesulfonamide was obtained. The product can be used directly in the next reaction without purification; ESI-MS(m/z): 609.0 [M+H]⁺.

### Step g): Preparation of N-(5-bromo-2-(trifluoromethoxy)pyridin-3-yl)-2,4-difluorobenzenesulfonamide

The crude product of N-(5-bromo-2-(trifluoromethoxy)pyridin-3-yl)-N-((2,4-difluorophenyl)sulfonyl)-2,4-difluorobenzenesulfonamide (500 mg), tetrahydrofuran (6 mL) and water (6 mL) mixed solvent were added to the reaction flask, stirred to dissolve, with 6 M potassium hydroxide aqueous solution (6 mL) added. The reaction mixture was stirred at room temperature for 1 h. Upon completion of the reaction, the reaction mixture was extracted by adding dichloromethane (30 mL) and water (20 mL). After that, the aqueous phase was extracted with dichloromethane (20 mL×2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=10/1). N-(5-bromo-2-(trifluoromethoxy)pyridin-3-yl)-2,4-difluorobenzenesulfonamide was obtained, yield: 70.1%; ESI-MS(m/z): 432.8 [M+H]⁺.

### Step h): Preparation of tert-butyl 4-(6-(5-((2,4-difluorophenyl)sulfonamido)-6-(trifluoromethoxy)pyridin-3-yl)quinazolin-4-yl)piperazine-1-carboxylate

N-(5-bromo-2-(trifluoromethoxy)pyridin-3-yl)-2,4-difluorobenzenesulfonamide (180 mg, 0.416 mmol), tert-butyl 4-(6-(4,4,5,5-tetramethyl-1,3,2-dioxaborolan-2-yl)quinazolin-4-yl)piperazine-1-carboxylate (202 mg, 0.458 mmol), Pd(dppf)Cl₂ (31 mg, 0.042 mmol) and cesium carbonate (406 mg, 1.248 mmol), dioxane (16 mL) and water (2 mL) were added to the reaction flask. After the addition was completed, in the presense of protective nitrogen, the reaction mixture was kept at 100° C. and stirred for 3 h. Upon completion of the reaction, the reaction mixture was cooled to room temperature, the reaction was quenched by adding saturated ammonium chloride (80 mL). The reaction mixture was extracted with ethyl acetate (80 mL). The organic phases were combined, dried over anhydrous sodium sulfate and filtered. The filtrate was concentrated under reduced pressure. The crude product obtained was purified by silica gel column chromatography (Eluent: dichloromethane/methanol=1/0 to 35/1). Tert-butyl 4-(6-(5-((2,4-difluorophenyl)sulfonamido)-6-(trifluoromethoxy)pyridin-3-yl)quinazolin-4-yl)piperazine-1-carboxylate was obtained, yield: 84.3%; ESI-MS(m/z): 667.5 [M+H]⁺.

### Step i): Preparation of 2,4-difluoro-N-(5-(4-(piperazin-1-yl)quinazolin-6-yl)-2-(trifluoromethoxy)pyridin-3-yl)benzenesulfonamide trifluoroacetate

Tert-butyl 4-(6-(5-((2,4-difluorophenyl)sulfonamido)-6-(trifluoromethoxy)pyridin-3-yl)quinazolin-4-yl)piperazine-1-carboxylate (347 mg, 0.521 mmol) was dissolved in dichloromethane (9 mL). Under the ice bath condition, TFA (3 mL) was slowly added dropwise. After the addition was completed, the reaction mixture was stirred at room temperature for 1 h. Upon completion of the reaction, the reaction mixture was concentrated under reduced pressure. The crude product obtained was dispersed in methyl tert-butyl ether (10 mL). The reaction mixture was stirred at room temperature for 0.5 h and filtered. The filter cake was dried to obtain 2,4-difluoro-N-(5-(4-(piperazin-1-yl)quinazolin-6-yl)-2-(trifluoromethoxy)pyridin-3-yl)benzenesulfonamide trifluoroacetate, yield: 96.5%; ESI-MS(m/z): 567.5 [M+H]⁺.

### Step j): Preparation of (L)-2,4-difluoro-N-(5-(4-(4-(4-oxopent-2-enoyl)piperazin-1-yl)quinazolin-6-yl)-2-(trifluoromethoxy)pyridin-3-yl)benzenesulfonamide

2,4-Difluoro-N-(5-(4-(piperazin-1-yl)quinazolin-6-yl)-2-(trifluoromethoxy)pyridin-3-yl)benzenesulfonamide trifluoroacetate (119 mg, 0.179 mmol), (E)-4-oxopent-2-enoic acid (22 mg, 0.197 mmol), DIPEA (116 mg, 0.895 mmol) were dissolved in tetrahydrofuran (2 mL). At −78° C., 1-propylphosphonic anhydride (171 mg, 0.269 mmol, 50% wt ethyl acetate solution) was slowly added to the system. After the addition was completed, the reaction mixture was kept at −78° C. and stirred for 1 h. Upon completion of the reaction, the reaction was quenched by adding saturated ammonium chloride (40 mL), and the reaction mixture was extracted with dichloromethane (40 mL×2). The organic phases were combined, dried over anhydrous sodium sulfate and filtered. The filtrate was concentrated under reduced pressure. The crude product obtained was purified by Prep-HPLC (Method 2). (E)-2,4-difluoro-N-(5-(4-(4-(4-oxopent-2-enoyl)piperazin-1-yl)quinazolin-6-yl)-2-(trifluoromethoxy)pyridin-3-yl)benzenesulfonamide was obtained, yield: 29.6%; ¹H NMR (400 MHz, DMSO-d₆) δ 10.96 (br s, 1H), 8.67 (s, 1H), 8.23-7.96 (m, 4H), 7.95-7.88 (m, 1H), 7.86-7.75 (m, 1H), 7.48-7.30 (m, 2H), 7.18-7.09 (m, 1H), 6.73 (d, J=15.6 Hz, 1H), 3.92-3.76 (m, 8H), 2.36 (s, 3H); ESI-MS(m/z): 663.0 [M+H]⁺.

### Example 195

### Preparation of (E)-N-(2-(dimethylamino)-5-(4-(4-(4-oxopent-2-enoyl)piperazin-1-yl)quinazolin-6-yl)pyridin-3-yl)-2,4-difluorobenzenesulfonamide

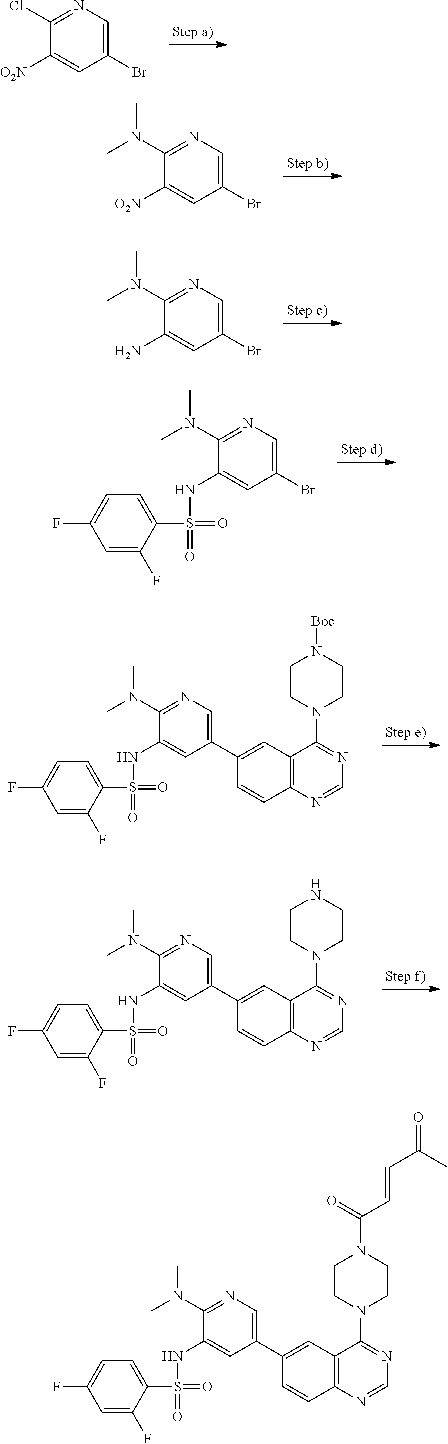

### Step a): Preparation of 5-bromo-N,N-dimethyl-3-nitropyridin-2-amine

5-Bromo-2-chloro-3-nitropyridine (550 mg, 2.316 mmol), DIPEA (748 mg, 5.790 mmol) and N,N-dimethylformamide (30 mL) were added to the reaction flask, stirred to dissolve, with dimethylamine hydrochloride (283 mg, 3.474 mmol) added. After the addition was completed, the reaction mixture was heated to 100° C. and reacted for 2 h. Upon completion of the reaction, the reaction was quenched by adding water (50 mL). The reaction mixture was extracted with ethyl acetate (150 mL×2). The organic phases were combined, washed with saturated brine (50 mL), dried over anhydrous sodium sulfate and filtered. The filtrate was concentrated under reduced pressure. The crude product obtained was purified by silica gel column chromatography (Eluent: petroleum ether/ethyl acetate=10/1 to 5/1), 5-Bromo-N,N-dimethyl-3-nitropyridin-2-amine was obtained, yield: 87.7%; ESI-MS(m/z): 246.0 [M+H]⁺.

### Step b): Preparation of 5-bromo-N,N-dimethylpyridine-2,3-diamine

5-Bromo-N,N-dimethyl-3-nitropyridin-2-amine (500 mg, 2.032 mmol), iron powder (567 mg, 10.160 mmol) and ammonium chloride (109 mg, 2.032 mmol) were added to methanol and water mixed solvent (12.5 mL, v/v=4/1). After the addition was completed, the reaction mixture was heated to 60° C., stirred and reacted for 4 h. Upon completion of the reaction, the reaction mixture was filtered. The filtrate was concentrated under reduced pressure, diluted by adding water (50 mL), extracted with dichloromethane (100 mL×3). The organic phases were combined, washed with saturated brine (50 mL), dried over anhydrous sodium sulfate and filtered. The filtrate was concentrated under reduced pressure. The crude product obtained was purified by silica gel column chromatography (Eluent: ethyl acetate/petroleum ether=5/1 to 2/1), 5-Bromo-N,N²-dimethylpyridine-2,3-diamine was obtained, yield: 91.1%; ESI-MS(m/z): 216.0 [M+H]⁺.

### Step c): Preparation of N-(5-bromo-2-(dimethylamino)pyridin-3-yl)-2,4-difluorobenzenesulfonamide

5-Bromo-N²,N²-dimethylpyridine-2,3-diamine (400 mg, 1.851 mmol) was dissolved in pyridine (10 mL), with 2,4-difluorobenzenesulfonyl chloride (590 mg, 2.777 mmol) added. The reaction mixture was kept at room temperature and reacted for 4 h. Upon completion of the reaction, the reaction was quenched by adding water (100 mL). The reaction mixture was extracted with ethyl acetate (100 mL). The organic phases were combined, washed with saturated brine (100 mL), dried over anhydrous sodium sulfate and filtered. The filtrate was concentrated under reduced pressure. The crude product obtained was purified by silica gel column chromatography (Eluent: dichloromethane/methanol=20/1 to 10/1). N-(5-bromo-2-(dimethylamino)pyridin-3-yl)-2,4-difluorobenzenesulfonamide was obtained, yield: 22.0%; ESI-MS(m/z): 392.0 [M+H]⁺.

### Step d): Preparation of tert-butyl 4-(6-(5-((2,4-difluorophenyl)sulfonamido)-6-(dimethylamino)pyridin-3-yl)quinazolin-4-yl)piperazine-1-carboxylate

N-(5-bromo-2-(dimethylamino)pyridin-3-yl)-2,4-difluorobenzenesulfonamide (160 mg, 0.408 mmol), tert-butyl 4-(6-(4,4,5,5-tetramethyl-1,3,2-dioxaborolan-2-yl)quinazolin-4-yl)piperazine-1-carboxylate (216 mg, 0.490 mmol), Pd(dppf)Cl₂ (30 mg, 0.041 mmol) and cesium carbonate (399 mg, 1.224 mmol) were added to dioxane (8 mL) and water (2 mL) mixed solvent. After the addition was completed, in the presense of protective nitrogen, the reaction mixture was heated to 110° C. and reacted for 2 h. Upon completion of the reaction, the reaction was quenched by adding water (30 mL). The reaction mixture was extracted with ethyl acetate (50 mL). The organic phases were combined, washed with saturated brine (30 mL), dried over anhydrous sodium sulfate and filtered. The filtrate was concentrated under reduced pressure. The crude product obtained was purified by silica gel column chromatography (Eluent: methanol/dichloromethane=1/30 to 1/10). Tert-butyl 4-(6-(5-((2,4-difluorophenyl)sulfonamido)-6-(dimethylamino)pyridin-3-yl)quinazolin-4-yl)piperazine-1-carboxylate was obtained, yield: 76.3%; ESI-MS(m/z): 626.2 [M+H]⁺.

### Step e): Preparation of N-(2-(dimethylamino)-5-(4-(piperazin-1-yl)quinazolin-6-yl)pyridin-3-yl)-2,4-difluorobenzenesulfonamide trifluoroacetate

Tert-butyl 4-(6-(5-((2,4-difluorophenyl)sulfonamido)-6-(dimethylamino)pyridin-3-yl)quinazolin-4-yl)piperazine-1-carboxylate (200 mg, 0.320 mmol) was dissolved in dichloromethane (4 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. N-(2-(dimethylamino)-5-(4-(piperazin-1-yl)quinazolin-6-yl)pyridin-3-yl)-2,4-difluorobenzenesulfonamide trifluoroacetate was obtained; ESI-MS(m/z): 526.2 [M+H]⁺.

### Step f): Preparation of (E)-N-(2-(dimethylamino)-5-(4-(4-(4-oxopent-2-enoyl)piperazin-1-yl)quinazolin-6-yl)pyridin-3-yl)-2,4-difluorobenzenesulfonamide

N-(2-(dimethylamino)-5-(4-(piperazin-1-yl)quinazolin-6-yl)pyridin-3-yl)-2,4-difluorobenzenesulfonamide trifluoroacetate (100 mg, 0.156 mmol) and tetrahydrofuran (5 mL) were added to the reaction flask. With the reaction system cooled to −78° C., DIPEA (102 mg, 0.780 mmol), (E)-4-oxopent-2-enoic acid (20 mg, 0.172 mmol) and 50% T₃P ethyl acetate solution (204 mg, 0.312 mmol, 50% wt) were added successively. After the addition was completed, the reaction mixture was kept at −78° C. and stirred for 1 h. Upon completion of the reaction, at −78° C., the reaction was quenched using saturated aqueous ammonium chloride solution (50 mL). The reaction mixture was extracted by adding dichloromethane (100 mL). The organic phases were combined, washed with saturated brine (100 mL), dried over anhydrous sodium sulfate and filtered. The filtrate was concentrated under reduced pressure. The crude product obtained was purified by Prep-HPLC (Method 2). (E)-N-(2-(dimethylamino)-5-(4-(4-(4-oxopent-2-enoyl)piperazin-1-yl)quinazolin-6-yl)pyridin-3-yl)-2,4-difluorobenzenesulfonamide was obtained, yield: 24.0%; ¹H NMR (400 MHz, DMSO-d₆) δ 8.63 (s, 1H), 8.02-7.96 (m, 1H), 7.95-7.76 (m, 4H), 7.56 (d, J=2.4 Hz, 1H), 7.46 (d, J=15.6 Hz, 1H), 7.29-7.20 (m, 1H), 7.12-7.02 (m, 1H), 6.73 (d, J=16.0 Hz, 1H), 3.95-3.72 (m, 8H), 2.94 (s, 6H), 2.37 (s, 3H); ESI-MS(m/z): 622.0 [M+H]⁺.

### Example 196

### Preparation of (E)-2,4-difluoro-N-(5-(8-fluoro-4-(4-(4-oxopent-2-enoyl)piperazin-1-yl)quinazoline-6-(yl)-2-methoxypyridin-3-yl)benzenesulfonamide

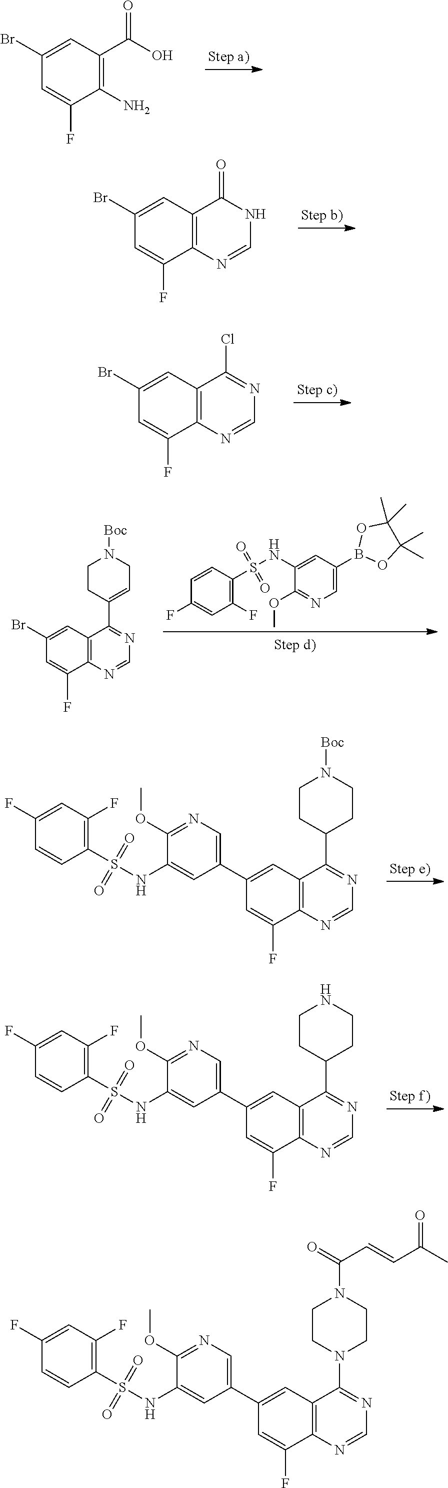

### Step a): Preparation of 6-bromo-8-fluoroquinazolin-4 (3H)-one

2-Amino-5-bromo-3-fluorobenzoic acid (500 mg, 2.137 mmol) was added to formamide (8 mL). The reaction mixture was microwave heated to 180° C. and reacted for 1 h. Upon completion of the reaction, the reaction solution was directly filtered. The filter cake was washed by water (10 mL×3), dried to obtain 6-bromo-8-fluoroquinazolin-4 (3H)-one, yield: 96.3%; ESI-MS(m/z): 243.0 [M+H]⁺.

### Step b): Preparation of 6-bromo-4-chloro-8-fluoroquinazoline

6-Bromo-8-fluoroquinazolin-4 (3H)-one (500 mg, 2.057 mmol) was added to thionyl chloride (10 mL), with DMF (8 mg, 0.103 mmol) added. The reaction mixture was heated to reflux and reacted for 2 h. Upon completion of the reaction, the reaction solution was directly concentrated under reduced pressure. 6-Bromo-4-chloro-8-fluoroquinazoline was obtained, yield: 92.9%; ESI-MS(m/z): 261.0 [M+H]⁺.

### Step c): Preparation of tert-butyl 4-(6-bromo-8-fluoroquinazolin-4-yl)piperazine-1-carboxylate

6-Bromo-4-chloro-8-fluoroquinazoline (200 mg, 0.765 mmol), tert-butyl piperazine-1-carboxylate (210 mg, 1.128 mmol), DIPEA (562 mg, 4.325 mmol) were added to DMF (10 mL). The reaction mixture was heated to 60° C. and reacted for 2 h. Upon completion of the reaction, the reaction was quenched by adding water (30 mL). The reaction mixture was extracted with ethyl acetate (50 mL 2). The organic phases were combined, washed with saturated brine (30 mL), dried over anhydrous sodium sulfate and filtered. The filtrate was concentrated under reduced pressure. The crude product obtained was purified by silica gel column chromatography (Eluent: methanol/dichloromethane=1/100 to 1/50). Tert-butyl 4-(6-bromo-8-fluoroquinazolin-4-yl)piperazine-1-carboxylate was obtained, yield: 95.2%; ESI-MS(m/z): 411.0 [M+H]⁺.

### Step d): Preparation of tert-butyl 4-(6-(5-((2,4-difluorophenyl)sulfonamido)-6-methoxypyridin-3-yl)-8-fluoroquinazolin-4-yl)piperazine-1-carboxylate

Tert-butyl 4-(6-bromo-8-fluoroquinazolin-4-yl)piperazine-1-carboxylate (300 mg, 0.729 mmol), 2,4-difluoro-N-(2-methoxy-5-(4,4,5,5-tetramethyl-1,3,2-dioxaborolan-2-yl)pyridin-3-yl)benzenesulfonamide (466 mg, 1.094 mmol), Pd(dppf)Cl₂ (53 mg, 0.073 mmol) and cesium carbonate (712 mg, 2.187 mmol) were added to dioxane/water mixed solvent (11 mL, v/v=10:1). After the addition was completed, in the presense of protective nitrogen, the reaction mixture was heated to reflux and reacted for 3 h. Upon completion of the reaction, the reaction was quenched by adding water (50 mL). The reaction mixture was extracted with ethyl acetate (50 mL×2). The organic phases were combined, washed with saturated brine (30 mL), dried over anhydrous sodium sulfate and filtered. The filtrate was concentrated under reduced pressure. The crude product obtained was purified by silica gel column chromatography (Eluent: methanol/dichloromethane=1/100 to 1/30). Tert-butyl 4-(6-(5-((2,4-difluorophenyl)sulfonamido)-6-methoxypyridin-3-yl)-8-fluoroquinazolin-4-yl)piperazine-1-carboxylate was obtained, yield: 65.2%; ESI-MS(m/z): 631.2 [M+H]⁺.

### Step e): Preparation of 2,4-difluoro-N-(5-(8-fluoro-4-(piperazin-1-yl)quinazolin-6-yl)-2-methoxypyridin-3-yl)benzenesulfonamide trifluoroacetate

Tert-butyl 4-(6-(5-((2,4-difluorophenyl)sulfonamido)-6-methoxypyridin-3-yl)-8-fluoroquinazolin-4-yl)piperazine-1-carboxylate (300 mg, 0.476 mmol) was dissolved in dichloromethane (4 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. The crude product was slurried with methyl tert-butyl ether (5 mL) to precipitate solid and filtered. 2,4-Difluoro-N-(5-(8-fluoro-4-(piperazin-1-yl)quinazolin-6-yl)-2-methoxypyridin-3-yl)benzenesulfonamide trifluoroacetate was obtained, yield: 97.1%; ESI-MS(m/z): 531.1 [M+H]⁺.

### Step f): Preparation of (E)-2,4-difluoro-N-(5-(8-fluoro-4-(4-(4-oxopent-2-enoyl)piperazin-1-yl)quinazoline-6-yl)-2-methoxypyridin-3-yl)benzenesulfonamide

2,4-Difluoro-N-(5-(8-fluoro-4-(piperazin-1-yl)quinazolin-6-yl)-2-methoxypyridin-3-yl)benzenesulfonamide trifluoroacetate (150 mg, 0.233 mmol) and tetrahydrofuran (5 mL) were added to the reaction flask. The reaction system was cooled to −78° C. with DIPEA (149 mg, 1.165 mmol), (E)-4-oxopent-2-enoic acid (29 mg, 0.256 mmol) and 50% T₃P ethyl acetate solution (293 mg, 0.466 mmol, 50% wt) added successively. After the addition was completed, the reaction mixture was kept at −78° C. and stirred for 1 h. Upon completion of the reaction, the reaction was quenched using saturated aqueous ammonium chloride solution (50 mL) at −78° C. The reaction mixture was extracted by adding dichloromethane (100 mL/2). The organic phases were combined, washed with saturated brine (100 mL), dried over anhydrous sodium sulfate and filtered. The filtrate was concentrated under reduced pressure. The crude product obtained was purified by Prep-HPLC (Method 2). (E)-2,4-difluoro-N-(5-(8-fluoro-4-(4-(4-oxopent-2-enoyl)piperazin-1-yl)quinazoline-6-yl)-2-methoxypyridin-3-yl)benzenesulfonamide was obtained, yield: 15.0%; ¹H NMR (400 MHz, DMSO-d₆) δ 10.15 (brs, 1H), 8.68 (s, 1H), 8.35 (s, 1H), 8.04-7.88 (m, 3H), 7.82-7.72 (m, 1H), 7.54-7.41 (m, 2H), 7.22-7.12 (m, 11H), 6.74 (d, J=16.0 Hz, 1H), 4.06-3.74 (m, 811), 3.69 (s, 3H), 2.37 (s, 3H); ESI-MS(m/z): 627.0 [M+H]⁺.

### Example 197

### Preparation of (E)-2,4-Difluoro-N-(2-methoxy-5-(4-(1-(4-oxopent-2-enoyl)piperidin-4-yl)quinazoline-6-yl)pyridin-3-yl)benzenesulfonamide

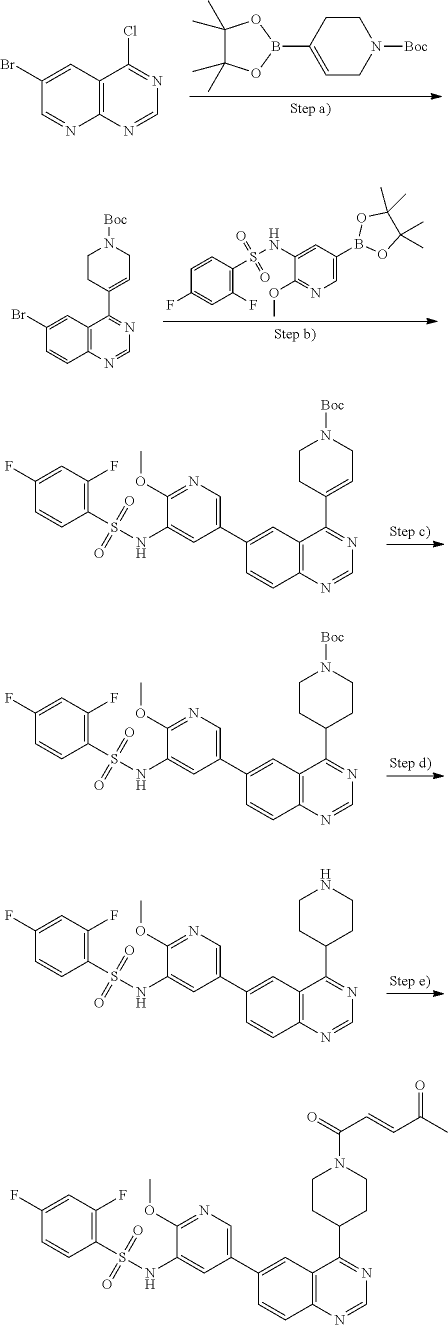

### Step a): Preparation of tert-butyl 4-(6-bromoquinazolin-4-yl)-3,6-dihydropyridine-1 (2H)-carboxylate

6-Bromo-4-chloroquinazoline (500 mg, 2.053 mmol), tert-butyl 4-(4,4,5,5-tetramethyl-1,3,2-dioxaborolan-2-yl)-3,6-dihydropyridine-1 (2H)-carboxylate (634.81 mg, 2.053 mmol), Pd(dppf)Cl₂ (150 mg, 0.205 mmol), sodium carbonate (653 mg, 6.159 mmol), dioxane (25 mL) and water (7 mL) were added to the reaction flask. After the addition was completed, in the presense of protective nitrogen, the reaction mixture was heated to 45° C. and stirred for 6 h. Upon completion of the reaction, the reaction mixture was cooled to room temperature and the reaction was quenched by adding saturated ammonium chloride (80 mL). The reaction mixture was extracted with ethyl acetate (80 mL 2). The organic phases were combined, dried over anhydrous sodium sulfate and filtered. The filtrate was concentrated under reduced pressure. The crude product obtained was purified by silica gel column chromatography (Eluent: dichloromethane/methanol=1/0 to 35/1). Tert-butyl 4-(6-bromoquinazolin-4-yl)-3,6-dihydropyridine-1 (2H)-carboxylate was obtained, yield: 48.0%; ESI-MS(m/z): 390.0 [M+H]⁺.

### Step b): Preparation of tert-butyl 4-(6-(5-((2,4-difluorophenyl)sulfonamido)-6-methoxypyridin-3-yl)quinazolin-4-yl)-3,6-dihydropyridine-1 (2H)-carboxylate

Tert-butyl 4-(6-bromoquinazolin-4-yl)-3,6-dihydropyridine-1 (2H)-carboxylate (256 mg, 0.656 mmol), 2,4-difluoro-N-(2-methoxy-5-(4,4,5,5-tetramethyl-1,3,2-dioxaborolan-2-yl)pyridin-3-yl)benzenesulfonamide (308 mg, 0.722 mmol), Pd(dppf)Cl₂ (48 mg, 0.066 mmol), cesium carbonate (640 mg, 1.968 mmol), dioxane (16 mL) and water (2 mL) were added to the reaction flask. After the addition was completed, in the presense of protective nitrogen, the reaction mixture was kept at 100° C. and stirred for 3 h. Upon completion of the reaction, the reaction mixture was cooled to room temperature and the reaction was quenched by adding saturated ammonium chloride (80 mL). The reaction mixture was extracted with ethyl acetate (80 mL×2). The organic phases were combined, dried over anhydrous sodium sulfate and filtered. The filtrate was concentrated under reduced pressure. The crude product obtained was purified by silica gel column chromatography (Eluent: dichloromethane/methanol=1/0 to 35/1), tert-butyl 4-(6-(5-((2,4-difluorophenyl)sulfonamido)-6-methoxypyridin-3-yl)quinazolin-4-yl)-3,6-dihydropyridine-1 (2H)-carboxylate was obtained, yield: 90.0%; ESI-MS(m/z): 610.6 [M+H]⁺.

### Step c): Preparation of tert-butyl 4-(6-(5-((2,4-difluorophenyl)sulfonamido)-6-methoxypyridin-3-yl)quinazolin-4-yl)piperidine-1-carboxylate

Tert-butyl 4-(6-(5-((2,4-difluorophenyl)sulfonamido)-6-methoxypyridin-3-yl)quinazolin-4-yl)-3,6-dihydropyridine-1 (2H)-carboxylate (150 mg, 0.246 mmol) was dissolved in methanol (6 mL), with 10% Pd/C (150 mg, 55% water) and acetic acid (150 mg, 2.500 mmol) added. After the addition was completed, under hydrogen atmosphere, the reaction mixture was kept at 25° C. and stirred for 5.5 h. Upon completion of the reaction, the reaction mixture was filtered. The filtrate was concentrated under reduced pressure. The crude product obtained was purified by silica gel column chromatography (Eluent: dichloromethane/methanol=1/0 to 35/1). Tert-butyl 4-(6-(5-((2,4-difluorophenyl)sulfonamido)-6-methoxypyridin-3-yl)quinazolin-4-yl)piperidine-1-carboxylate was obtained, yield: 30.6%; ESI-MS(m/z): 612.0 [M+H]⁺.

### Step d): Preparation of 2,4-difluoro-N-(2-methoxy-5-(4-(piperidin-4-yl)quinazolin-6-yl)pyridin-3-yl)benzenesulfonamide trifluoroacetate

Tert-butyl 4-(6-(5-((2,4-difluorophenyl)sulfonamido)-6-methoxypyridin-3-yl)quinazolin-4-yl)piperidine-1-carboxylate (56 mg, 0.092 mmol) was dissolved in dichloromethane (3 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. The crude product obtained was dispersed in methyl tert-butyl ether (10 mL). The reaction mixture was stirred at room temperature for 0.5 h and filtered. The filter cake was vacuum dried to obtain 2,4-difluoro-N-(2-methoxy-5-(4-(piperidin-4-yl)quinazolin-6-yl)pyridin-3-yl)benzenesulfonamide trifluoroacetate, yield: 91.2%; ESI-MS(m/z): 512.0 [M+H]⁺.

### Step e): Preparation of (E)-2,4-Difluoro-N-(2-methoxy-5-(4-(1-(4-oxopent-2-enoyl)piperidin-4-yl)quinazoline-6-yl)pyridin-3-yl)benzenesulfonamide

2,4-Difluoro-N-(2-methoxy-5-(4-(piperidin-4-yl)quinazolin-6-yl)pyridin-3-yl)benzenesulfonamide trifluoroacetate (56 mg, 0.090 mmol), (E)-4-oxopent-2-enoic acid (12 mg, 0.099 mmol), DIPEA (58 mg, 0.450 mmol) were dissolved in tetrahydrofuran (2 mL). At −78° C., 1-propylphosphonic anhydride (86 mg, 0.135 mmol, 50% wt) was slowly added to the system. After the addition was completed, the reaction mixture was kept at −78° C. and stirred for 1 h. Upon completion of the reaction, the reaction was quenched using saturated aqueous ammonium chloride solution (20 mL) at −78° C. The reaction mixture was extracted by adding dichloromethane (20 mL×2). The organic phases were combined, washed with saturated brine (20 mL), dried over anhydrous sodium sulfate and filtered. The filtrate was concentrated under reduced pressure. The crude product obtained was purified by Prep-HPLC (Method 2). (E)-2,4-Difluoro-N-(2-methoxy-5-(4-(1-(4-oxopent-2-enoyl)piperidin-4-yl)quinazoline-6-yl)pyridin-3-yl)benzenesulfonamide was obtained, yield: 21.0%; ¹H NMR (400 MHz, DMSO-d₆) δ 10.24 (brs, 1H), 9.14 (s, 1H), 8.54 (d, J=2.4 Hz, 1H), 8.41 (s, 1H), 8.19-8.12 (m, 1H), 8.03 (d, J=8.4 Hz, 1H), 7.97 (s, 1H), 7.76-7.66 (m, 1H), 7.49-7.37 (m, 2H), 7.17-7.09 (m, 1H), 6.63 (d, J=15.6 Hz, 1H), 4.53 (d, J=12.8 Hz, 1H), 4.24-4.11 (m, 2H), 3.63 (s, 3H), 3.38 (t, J=12.8 Hz, 1H), 2.97 (t, J=12.4 Hz, 1H), 2.30 (s, 3H), 1.97-1.66 (m, 4H); ESI-MS(m/z): 608.0 [M+H]+.

### Example 198

### Preparation of (E)-2,4-difluoro-N-(2-methoxy-5-(4-(1-(4-oxopent-2-enoyl)-1,2,3,6-tetrahydropyridin-4-yl)quinazolin-6-yl)pyridin-3-yl)benzenesulfonamide

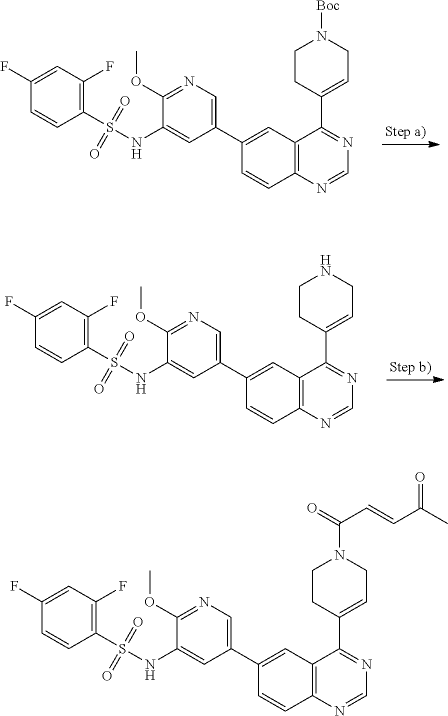 [SMILES: COc1ncc(-c2ccc3ncnc(C4=CCN(C(=O)OC(C)(C)C)CC4)c3c2)cc1NS(=O)(=O)c1ccc(F)cc1F ; COc1ncc(-c2ccc3ncnc(C4=CCNCC4)c3c2)cc1NS(=O)(=O)c1ccc(F)cc1F ; COc1ncc(-c2ccc3ncnc(C4=CCN(C(=O)/C=C/C(C)=O)CC4)c3c2)cc1NS(=O)(=O)c1ccc(F)cc1F]

### Step a): Preparation of 2,4-difluoro-N-(2-methoxy-5-(4-(1,2,3,6-tetrahydropyridin-4-yl)quinazolin-6-yl)pyridine-3-benzenesulfonamide trifluoroacetate

tert-butyl 4-(6-(5-((2,4-difluorophenyl)sulfonamido)-6-methoxypyridin-3-yl)quinazolin-4-yl)-3,6-dihydropyridine-1 (2H)-carboxylate (50 mg, 0.082 mmol) was dissolved in dichloromethane (3 mL), under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. The crude product obtained was dispersed in methyl tert-butyl ether (10 mL). The reaction mixture was stirred at room temperature for 0.5 h and filtered. The filter cake was dried to obtain 2,4-difluoro-N-(2-methoxy-5-(4-(1,2,3,6-tetrahydropyridin-4-yl)quinazolin-6-yl)pyridine-3-benzenesulfonamide trifluoroacetate, yield: 84.2%; ESI-MS(m/z): 510.1 [M+H]⁺.

### Step b): Preparation of (E)-2,4-difluoro-N-(2-methoxy-5-(4-(1-(4-oxopent-2-enoyl)-1,2,3,6-tetrahydropyridin-4-yl)quinazolin-6-yl)pyridin-3-yl)benzenesulfonamide

2,4-Difluoro-N-(2-methoxy-5-(4-(1,2,3,6-tetrahydropyridin-4-yl)quinazolin-6-yl)pyridine-3-benzenesulfonamide trifluoroacetate (50 mg, 0.080 mmol), (E)-4-oxopent-2-enoic acid (12 mg, 0.104 mmol), DIPEA (52 mg, 0.400 mmol) were dissolved in tetrahydrofuran (2 mL). At −78° C., T₃P (56 mg, 0.088 mmol, 50% wt) was slowly added to the system. After the addition was completed, the reaction mixture was kept at −78° C. and stirred for 1 h. Upon completion of the reaction, at −78 C. the reaction was quenched using saturated aqueous ammonium chloride solution (20 mL). The reaction mixture was extracted by adding dichloromethane (20 mL×2). The organic phases were combined, washed with saturated brine (20 mL), dried over anhydrous sodium sulfate and filtered. The filtrate was concentrated under reduced pressure. The crude product obtained was purified by Prep-HPLC (Method 2). (E)-2,4-difluoro-N-(2-methoxy-5-(4-(1-(4-oxopent-2-enoyl)-1,2,3,6-tetrahydropyridin-4-yl)quinazolin-6-yl)pyridin-3-yl)benzenesulfonamide was obtained, yield: 11.4%; ¹H NMR (400 MHz, DMSO-d₆) δ 10.32 (brs, 1H), 9.25 (s, 11H), 8.47-8.39 (m, 2H), 8.31-8.24 (m, 1H), 8.12 (d, J=8.8 Hz, 1H), 8.04-7.98 (m, 1H), 7.84-7.70 (m, 1H), 7.58-7.41 (m, 2H), 7.23-7.14 (m, 1H), 6.74 (d, J=16.0 Hz, 1H), 6.51-6.41 (m, 1H), 4.57-4.35 (m, 2H), 3.98-3.87 (m, 2H), 3.70 (s, 3H), 2.87-2.72 (m, 2H), 2.37 (d, J=13.2 Hz, 3H); ESI-MS(m/z): 606.0 [M+H]⁺.

Referring to the preparation method of Example 198 and using the corresponding raw materials, the compounds in the following examples were obtained.

| Number | Name | Structure | ¹H NMR and MS |
|---|---|---|---|
| Example 199 | (E)-N-(2- methoxy-5-(4-(1- (4-oxopent-2- enoyl)-1,2,3,6- tetrahydropyridin- 4-yl)quinazolin-6- yl)pyridin-3-yl)-2,4- dimethylthiazole- 5-sulfonamide | 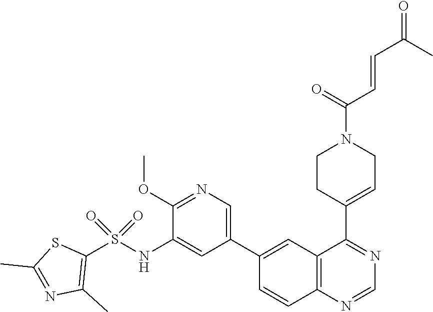 [SMILES: COc1ncc(-c2ccc3ncnc(C4=CCN(C(=O)/C=C/C(C)=O)CC4)c3c2)cc1NS(=O)(=O)c1sc(C)nc1C] | ¹H NMR (400 MHz, DMSO-d₆) δ 10.33 (brs, 1H), 9.25 (d, J = 1.6 Hz, 1H), 8.47-8.38 (m, 1H), 8.28-8.23 (m, 2H), 8.17-8.08 (m, 1H), 8.01- 7.88 (m, 1H), 7.51 (dd, J₁ = 15.6 H_{Z}, J₂ = 9.6 Hz, 1H), 6.73 (dd, J₁ = 15.6 H_{Z}, J₂ = 9.6 Hz, 1H), 6.51-6.40 (m, 1H), 4.55 (d, J = 3.2 H_{Z}, 1H), 4.40 (d, J = 3.2 H_{Z}, 1H), 4.03-3.86 (m, 2H), 3.77 (s, 3H), 2.84-2.77 (m, 2H), 2.54 (s, 3H), 2.45-2.29 (m, 6H); ESI-MS (m/z): 605.0 [M + H]⁺. |
| Example 200 | (E)-2-fluoro-N-(2- methoxy-5-(4-(1- (4-oxopent-2- enoyl)-1,2,3,6- tetrahydropyridin- 4-yl)quinazolin-6- yl)pyridin-3- yl)benzene- sulfonamide | 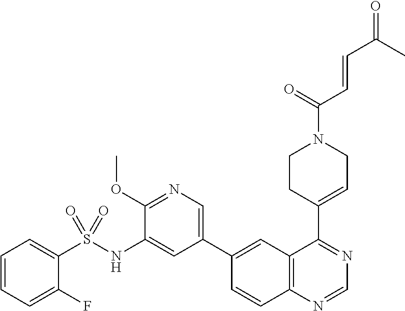 [SMILES: COc1ncc(-c2ccc3ncnc(C4=CCN(C(=O)/C=C/C(C)=O)CC4)c3c2)cc1NS(=O)(=O)c1ccccc1F] | ¹H NMR (400 MHz, DMSO-d₆) δ 10.23 (s, 1H), 9.25 (s, 1H), 8.42-8.34 (m, 2H), 8.25-8.22 (m, 1H), 8.11 (d, J = 4.4 Hz, 1H), 7.95 (s, 1H), 7.76- 7.69 (m, 1H), 7.62-7.48 (m, 2H), 7.44-7.37 (m, 1H), 7.27 (dd, J₁ = 15.2 Hz, J₂ = 7.6 Hz, 1H), 6.76-6.71 (m, 1H), 6.44 (d, J = 18.0 Hz, 1H), 4.54-4.38 (m, 2H), 3.96-3.89 (m, 2H), 3.69 (s, 3H), 2.84-2.77 (m, 2H), 2.39-2.34 (m, 3H); ESI-MS (m/z): 588.0 [M + H]⁺. |
| Example 201 | (E)-2,6-difluoro- N-(2-methoxy-5- (4-(1-(4-oxopent- 2-enoyl)-1,2,3,6- tetrahydropyridin- 4-yl)quinazolin-6- yl)pyridin-3- yl)benzenesulfonamide | 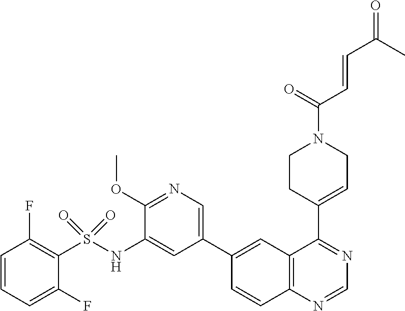 [SMILES: COc1ncc(-c2ccc3ncnc(C4=CCN(C(=O)/C=C/C(C)=O)CC4)c3c2)cc1NS(=O)(=O)c1c(F)cccc1F] | ¹H NMR (400 MHz, DMSO-d₆) δ 10.53 (s, 1H), 9.24 (s, 1H), 8.42 (dd, J₁ = 2.0 Hz, J₂ = 12.0 Hz, 1H), 8.26- 8.20 (m, 2H), 8.12-8.09 (m, 1H), 7.96 (d, J = 12.8 Hz, 1H), 7.58-7.45 (m, 2H), 7.20-7.11 (m, 2H), 6.76- 7.69 (m, 1H), 6.47-6.40 (m, 1H), 4.55-4.53 (m, 1H), 4.40-4.38 (m, 1H), 3.96-3.89 (m, 2H), 3.70 (d, J = 3.2 Hz, 3H), 2.84-2.77 (m, 2H), 2.39-2.36 (m, 3H); ESI-MS (m/z): 606.0 [M + H]⁺. |
| Example 202 | (E)-2-chloro-6- fluoro-N-(2- methoxy-5-(4-(1- (4-oxopent-2- enoyl)-1,2,3,6- tetrahydropyridin- 4-yl)quinazolin-6- yl)pyridin-3- yl)benzene- sulfonamide | 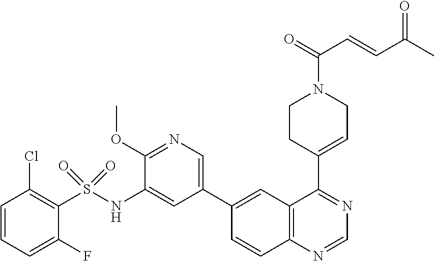 [SMILES: COc1ncc(-c2ccc3ncnc(C4=CCN(C(=O)/C=C/C(C)=O)CC4)c3c2)cc1NS(=O)(=O)c1c(F)cccc1Cl] | ¹H NMR (400 MHz, DMSO-d₆) δ 10.52 (s, 1H), 9.26 (d, J = 2.0 Hz, 1H), 8.54-8.47 (m, 1H), 8.47-8.41 (m, 1H), 8.33-8.23 (m, 1H), 8.13 (d, J = 8.8 Hz, 1H), 8.06 (dd, J₁ = 13.2 Hz, J₂ = 2.4 Hz, 1H), 7.70-7.60 (m, 1H), 7.60-7.34 (m, 3H), 6.74 (d, J = 16.0 Hz, 1H), 6.53-6.41 (m, 1H), 4.56-4.36 (m, 2H), 3.98-3.86 (m, 2H), 3.67 (s, 3H), 2.88-2.73 (m, 2H), 2.37 (d, J = 16.0 Hz, 3H); ESI-MS (m/z): 622.0 [M + H]⁺. |

### Example 203

### Preparation of (E)-N-(2-methoxy-S-(4-(1-(4-oxopent-2-enoyl)piperidin-4-yl)quinazolin-6-yl)pyridin-3-yl)-2,4-dimethylthiazole-5-sulfonamide

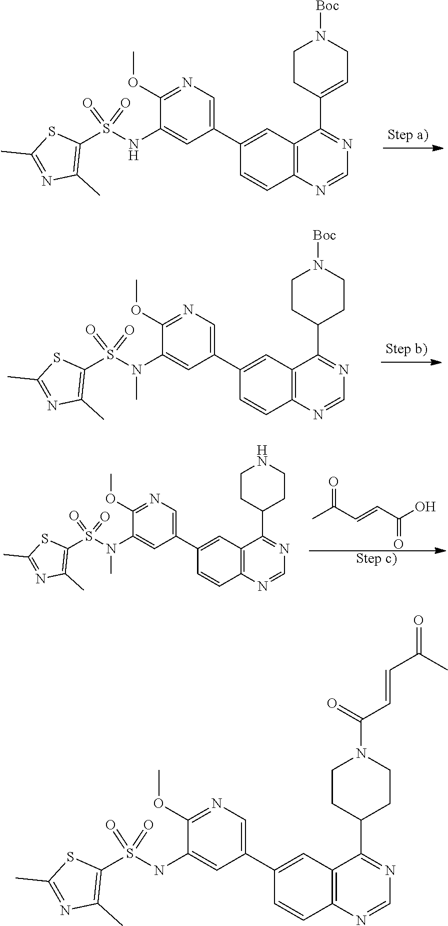

### Step a): Preparation of tert-butyl 4-(6-(5-((2,4-dimethylthiazole)-5-sulfonamido)-6-methoxypyridin-3-yl)quinazolin-4-yl)piperidine-1-carboxylate

Tert-butyl 4-(6-(5-((2,4-dimethylthiazole)-5-sulfonamido)-6-methoxypyridin-3-yl)quinazolin-4-yl)-3,6-dihydropyridine-1 (2H)-carboxylate (200 mg, 0.329 mmol) was added to methanol (10 mL). After that, glacial acetic acid (197 mg, 3.290 mmol) and 10% Pd/C (100 mg). After the addition was completed, under hydrogen atmosphere, the reaction mixture was kept at 30° C. and reacted for 5 h. Upon completion of the reaction, the reaction mixture was filtered. The filtrate was concentrated under reduced pressure. The crude product obtained was purified by silica gel column chromatography (Eluent: dichloromethane/methanol=100/1 to 50/1). Tert-butyl 4-(6-(5-((2,4-dimethylthiazole)-5-sulfonamido)-6-methoxypyridin-3-yl)quinazolin-4-yl)piperidine-1-carboxylate was obtained, yield: 39.4%; ESI-MS(m/z): 611.2 [M+H]⁺.

### Step b): Preparation of N-(2-methoxy-5-(4-(piperidin-4-yl)quinazolin-6-yl)pyridin-3-yl)-2,4-dimethylthiazole-5-sulfonamide trifluoroacetate

Tert-butyl 4-(6-(5-((2,4-dimethylthiazole)-5-sulfonamido)-6-methoxypyridin-3-yl)quinazolin-4-yl)piperidine-1-carboxylate (50 mg, 0.082 mmol) was dissolved in dichloromethane (4 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. The crude product was slurried with methyl tert-butyl ether (5 mL) to precipitate solid and filtered. N-(2-methoxy-5-(4-(piperidin-4-yl)quinazolin-6-yl)pyridin-3-yl)-2,4-dimethylthiazole-5-sulfonamide trifluoroacetate was obtained, yield: 88.3%; ESI-MS(m/z): 511.1 [M+H]⁺.

### Step c): Preparation of (E)-N-(2-methoxy-5-(4-(1-(4-oxopent-2-enoyl)piperidin-4-yl)quinazolin-6-yl)pyridin-3-yl)-2,4-dimethylthiazole-5-sulfonamide

N-(2-methoxy-5-(4-(piperidin-4-yl)quinazolin-6-yl)pyridin-3-yl)-2,4-dimethylthiazole-5-sulfonamide trifluoroacetate (30 mg, 0.048 mmol) and tetrahydrofuran (2 mL) were added to the reaction flask. The reaction system was cooled to −78° C., with N,N-diisopropylethylamine (11 mg, 0.288 mmol), (E)-4-oxopent-2-enoic acid (6 mg, 0.048 mmol) and 50% T₃P ethyl acetate solution (61 mg, 0.096 mmol, 50% wt) added successively. After the addition was completed, the reaction mixture was kept at −78° C. and stirred for 1 h. Upon completion of the reaction, the reaction solution was diluted by adding acetonitrile (1 mL), purified by Prep-HPLC (Method 2) and freeze dried. (E)-N-(2-methoxy-5-(4-(1-(4-oxopent-2-enoyl)piperidin-4-yl)quinazolin-6-yl)pyridin-3-yl)-2,4-dimethylthiazole-5-sulfonamide was obtained, yield: 19.5%; ¹H NMR (400 MHZ, DMSO-d₆) δ 10.27 (br s, 1H), 9.20 (s, 1H), 8.53 (s, 1H), 8.29 (s, 1H), 8.19-8.16 (m, 1H), 8.10 (d, J=8.7 Hz, 1H), 7.91 (s, 1H), 7.50 (d, J=15.6 Hz, 1H), 6.70 (d, J=15.6 Hz, 1H), 4.66-4.56 (m, 1H), 4.29-4.18 (m, 2H), 3.77 (s, 3H), 3.53-3.40 (m, 1H), 3.10-3.00 (m, 1H), 2.55 (s, 3H), 2.40 (s, 3H), 2.37 (s, 3H), 2.02-1.77 (m, 4H); ESI-MS (m/z): 607.0 [M+H]⁺.

Referring to the preparation method of Example 203 and using the corresponding raw materials, the compounds in the following examples were obtained.

| Number | Name | Structure | ¹H NMR and MS |
|---|---|---|---|
| Example 204 | (E)-2-fluoro-N-(2- methoxy-5-(4-(1- (4-oxopent-2- enoyl)piperidin-4- yl)quinazolin-6- yl)pyridin-3- yl)benzenesulfonamide | 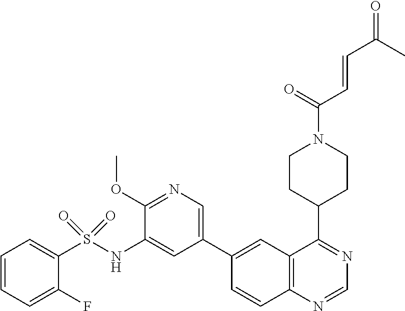 [SMILES: COc1ncc(-c2ccc3ncnc(C4CCN(C(=O)/C=C/C(C)=O)CC4)c3c2)cc1NS(=O)(=O)c1ccccc1F] | ¹H NMR (400 MHz, DMSO-d₆) δ 9.18 (s, 1H), 8.45 (s, 1H), 8.06 (s, 3H), 7.80-7.76 (m, 2H), 7.51-7.48 (m, 2H), 7.26-7.21 (m, 2H), 6.69 (d, J = 15.6 Hz, 1H), 4.61-4.58 (m, 1H), 4.27-4.14 (m, 2H), 3.76 (s, 3H), 3.51- 3.44 (m, 1H), 3.06-2.97 (m, 1H), 2.37 (s, 3H), 2.01-1.88 (m, 3H), 1.83-1.74 (m, 1H); ESI-MS (m/z): 590.0 [M + H]⁺. |
| Example 205 | (E)-2,6-difluoro- N-(2-methoxy-5-(4-(1- (4-oxopent-2-enoyl) piperidin-4-yl)quinazolin- 6-yl)pyridin-3-yl) benzenesulfonamide | 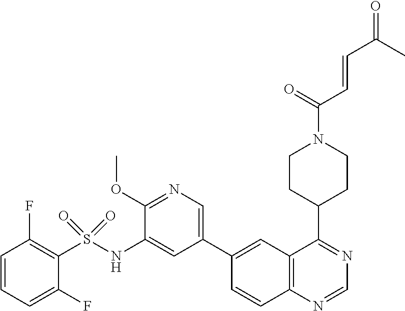 [SMILES: COc1ncc(-c2ccc3ncnc(C4CCN(C(=O)/C=C/C(C)=O)CC4)c3c2)cc1NS(=O)(=O)c1c(F)cccc1F] | ¹H NMR (400 MHz, DMSO-d₆) δ 10.61 (s, 1H), 9.21 (s, 1H), 8.60 (s, 1H), 8.42 (s, 1H), 8.22 (d, J = 8.8 Hz, 1H), 8.13-8.03 (m, 2H), 7.63 (s, 1H), 7.42 (d, J = 16.0 Hz, 1H), 7.24-7.19 (m, 2H), 6.70 (d, J = 16.0 Hz, 1H), 4.62-4.59 (m, 1H), 4.27-4.24 (m, 2H), 3.68 (s, 3H), 3.48-3.41 (m, 1H), 3.06- 3.00 (m, 1H), 2.37 (s, 3H), 2.01-1.78 (m, 4H); ESI-MS (m/z): 608.0 [M + H]⁺. |
| Example 206 | (E)-2-chloro-6-fluoro-N-(2- methoxy-5-(4-(1-(4- oxopent-2-enoyl)piperidin- 4-yl)quinazolin-6-yl) pyridin-3-yl) benzenesulfonamide | 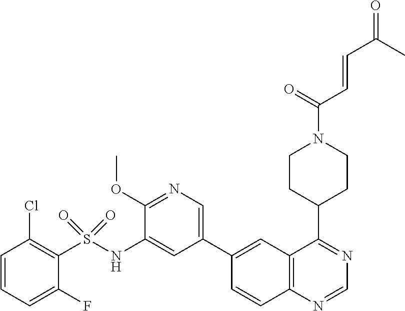 [SMILES: COc1ncc(-c2ccc3ncnc(C4CCN(C(=O)/C=C/C(C)=O)CC4)c3c2)cc1NS(=O)(=O)c1c(F)cccc1Cl] | ¹H NMR (400 MHz, DMSO-d₆) δ 9.20 (s, 1H), 8.56 (s, 1H), 8.35 (br s, 1H), 8.19 (d, J = 8.8 Hz, 1H), 8.09 (d, J = 8.8 Hz, 1H), 7.96 (s, 1H), 7.60- 7.29 (m, 4H), 6.70 (d, J = 15.6 Hz, 1H), 4.60 (d, J = 12.4 Hz, 1H), 4.31- 4.16 (m, 2H), 3.69 (s, 3H), 3.44 (t, J = 12.8 Hz, 1H), 3.09-2.97 (m, 1H), 2.37 (s, 3H), 2.05-1.72 (m, 4H); ESI-MS (m/z): 624.0 [M + H]⁺. |

### Example 207

### Preparation of 2,4-difluoro-N-(2-methoxy-5-(4-(4-(vinylsulfonyl) piperazin-1-yl) quinazolin-6-yl)pyridin-3-yl)benzenesulfonamide

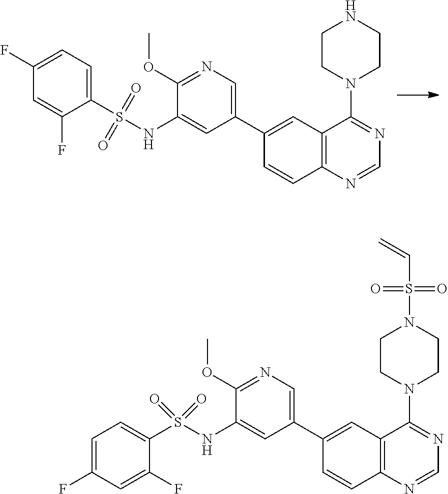 [SMILES: COc1ncc(-c2ccc3ncnc(N4CCNCC4)c3c2)cc1NS(=O)(=O)c1ccc(F)cc1F ; C=CS(=O)(=O)N1CCN(c2ncnc3ccc(-c4cnc(OC)c(NS(=O)(=O)c5ccc(F)cc5F)c4)cc23)CC1]

2,4-difluoro-N-(2-methoxy-5-(4-(piperazin-1-yl)quinazolin-6-yl)pyridin-3-yl)benzenesulfonamide trifluoroacetate (100 mg, 0.160 mmol) and tetrahydrofuran (5 mL) were added to the reaction flask. At 0° C., 4 A molecular sieve (200 mg) was added. After that, triethylamine (81 mg, 0.800 mmol) was slowly added to the system. In the presense of protective nitrogen, 2-chloroethane-1-sulfonyl chloride (20 mg, 0.124 mmol) tetrahydrofuran solution (0.5 mL) was added dropwise. After the addition was completed, the reaction mixture was kept at 0° C. and reacted for 1 h. Upon completion of the reaction, the reaction was quenched by adding saturated ammonium chloride (15 mL), and the reaction mixture was extracted with dichloromethane (15 mL/2). The organic phases were combined, dried over anhydrous sodium sulfate and filtered. The filtrate was concentrated under reduced pressure. The crude product obtained was purified by Prep-HPLC (Method 2), 2,4-Difluoro-N-(2-methoxy-5-(4-(4-(vinylsulfonyl)piperazin-1-yl)quinazolin-6-yl)pyridin-3-yl)benzenesulfonamide was obtained, yield: 38.2%; ¹H NMR (400 MHz, DMSO-d₆) δ 10.35 (s, 1H), 8.69 (s, 1H), 8.45 (s, 1H), 8.15-8.06 (m, 2H), 8.03-7.98 (m, 1H), 7.93 (d, J=8.4 Hz, 1H), 7.82-7.73 (m, 1H), 7.56 (t, J=9.6 Hz, 1H), 7.21 (dd, J=8.8, 2.4 Hz, 1H), 6.93-6.82 (m, 1H), 6.24-6.13 (m, 2H), 3.96-3.80 (m, 4H), 3.69 (s, 3H), 3.32-3.27 (m, 4H); ESI-MS(m/z): 503.1 [M+H]⁺.

### Example 208

### Preparation of (E)-N-(2-(difluoromethoxy)-5-(4-(4-(4-oxopent-2-enoyl)piperazin-1-yl)quinazoline-6-yl)pyridin-3-yl)-2,4-difluorobenzenesulfonamide

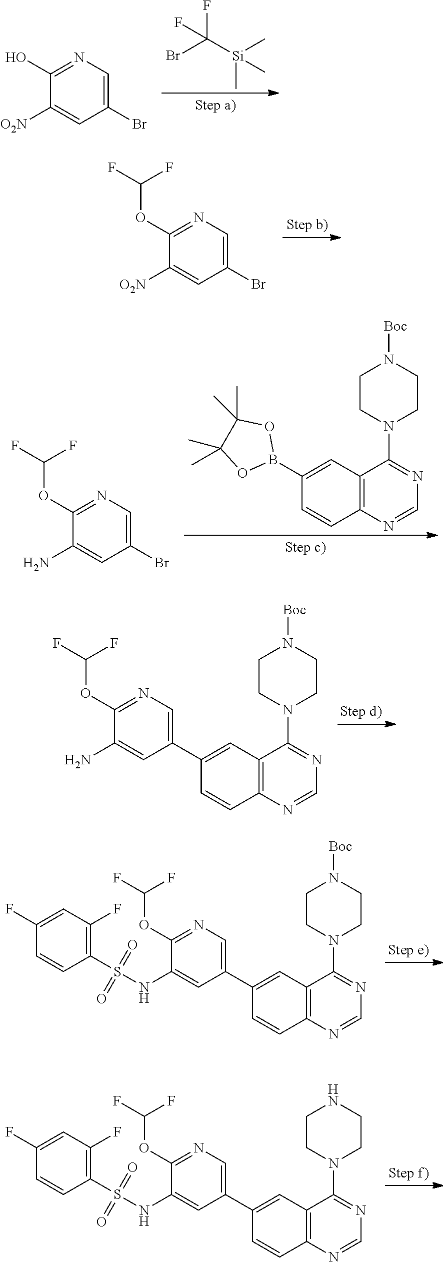

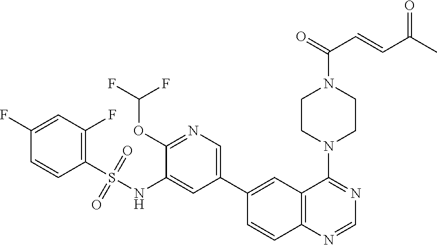 [SMILES: CC(=O)/C=C/C(=O)N1CCN(c2ncnc3ccc(-c4cnc(OC(F)F)c(NS(=O)(=O)c5ccc(F)cc5F)c4)cc23)CC1]

### Step a): Preparation of 5-bromo-2-(difluoromethoxy)-3-nitropyridine

5-Bromo-3-nitropyridin-2-ol (1.0 g, 4.566 mmol) and sodium carbonate (532 mg, 5.023 mmol) were added to acetonitrile (30 mL). The reaction mixture was heated to 60° C. and reacted for 15 min, with (bromodifluoromethyl)trimethylsilane (1.4 g, 6.849 mmol) slowly added to the reaction solution. After the addition was completed, the reaction mixture was heated to 75° C. and reacted for 4 h. Upon completion of the reaction, the reaction was quenched by adding water (30 mL). The reaction mixture was extracted with ethyl acetate (60 mL×2). The organic phases were combined, dried over anhydrous sodium sulfate and filtered. The filtrate was concentrated under reduced pressure. The residue was purified by silica gel column chromatography (Eluent: petroleum ether/ethyl acetate=3/1 to 1/1), 5-Bromo-2-(difluoromethoxy)-3-nitropyridine was obtained, yield: 28.5%; ESI-MS (m/z): 268.9 [M+H]⁺.

### Step b): Preparation of 5-bromo-2-(difluoromethoxy)pyridin-3-amine

5-Bromo-2-(difluoromethoxy)-3-nitropyridine (650 mg, 2.416 mmol) and Pt/C (700 mg, 0.179 mmol, 5% wt %) and ethyl acetate (10 mL) were added to the reaction flask. Under hydrogen atmosphere, the reaction mixture was kept at room temperature and reacted for 4 h. Upon completion of the reaction, the reaction solution was filtered. The filtrate was concentrated under reduced pressure. 5-Bromo-2-(difluoromethoxy)pyridin-3-amine was obtained, yield: 74.5%; ESI-MS(m/z): 239.0 [M+H]⁺.

### Step c): Preparation of tert-butyl 4-(6-(5-amino-6-(difluoromethoxy)pyridin-3-yl)quinazolin-4-yl)piperazine-1-carboxylate

5-Bromo-2-(difluoromethoxy)pyridin-3-amine (200 mg, 0.837 mmol), tert-butyl 4-(6-(4,4,5,5-tetramethyl-1,3,2-dioxaborolan-2-yl)quinazolin-4-yl)piperazine-1-carboxylate (442 mg, 1.004 mmol), Pd(dppf)Cl₂ (61 mg, 0.084 mmol), cesium carbonate (821 mg, 2.511 mmol), 1,4-dioxane (10 mL) and water (2 mL) were added to the reaction flask, in the presense of protective nitrogen, the reaction mixture was heated to reflux, stirred and reacted for 2 h. Upon completion of the reaction, the reaction solution was concentrated under reduced pressure. The residue was purified by silica gel column chromatography (Eluent: dichloromethane/methanol=20/1 to 10/1). Tert-butyl 4-(6-(5-amino-6-(difluoromethoxy)pyridin-3-yl)quinazolin-4-yl)piperazine-1-carboxylate was obtained, yield: 86.0/0; ESI-MS(m/z): 473.6 [M+H]⁺.

### Step d): Preparation of tert-butyl 4-(6-(6-(difluoromethoxy)-5-((2,4-difluorophenyl)sulfonamido)pyridin-3-yl)quinazolin-4-yl)piperazine-1-carboxylate

Tert-butyl 4-(6-(5-amino-6-(difluoromethoxy)pyridin-3-yl)quinazolin-4-yl)piperazine-1-carboxylate (360 mg, 0.762 mmol) and 2,4-difluorobenzenesulfonyl chloride (162 mg, 0.914 mmol) were dissolved in pyridine (8 mL). The reaction mixture was kept at room temperature and reacted overnight. Upon completion of the reaction, the reaction was quenched by adding water (50 mL). The reaction mixture was extracted by adding dichloromethane (100 mL). The organic phases were combined, washed with saturated brine (100 mL), dried over anhydrous sodium sulfate and filtered. The filtrate was concentrated under reduced pressure. The crude product obtained was purified by silica gel column chromatography (Eluent: petroleum ether/ethyl acetate=3/1 to 1/1). Tert-butyl 4-(6-(6-(difluoromethoxy)-5-((2,4-difluorophenyl)sulfonamido)pyridin-3-yl)quinazolin-4-yl)piperazine-1-carboxylate was obtained, yield: 54.7%; ESI-MS(m/z): 649.6 [M+H]⁺.

### Step e): Preparation of N-(2-(difluoromethoxy)-5-(4-(piperazin-1-yl)quinazolin-6-yl)pyridin-3-yl)-2,4-difluorobenzenesulfonamide trifluoroacetate

Tert-butyl 4-(6-(6-(difluoromethoxy)-5-((2,4-difluorophenyl)sulfonamido)pyridin-3-yl)quinazolin-4-yl)piperazine-1-carboxylate (270 mg, 0.416 mmol) was dissolved in dichloromethane (4 mL). Under the ice bath condition. TFA (I mL) was slowly added dropwise. After the addition was completed, the reaction mixture was stirred at room temperature for 1 h. Upon completion of the reaction, the reaction mixture was concentrated under reduced pressure. N-(2-(difluoromethoxy)-5-(4-(piperazin-1-yl)quinazolin-6-yl)pyridin-3-yl)-2,4-difluorobenzenesulfonamide trifluoroacetate was obtained; ESI-MS(m/z): 549.1 [M+H]⁺.

### Step f): Preparation of (E)-N-(2-(difluoromethoxy)-5-(4-(4-(4-oxopent-2-enoyl)piperazin-1-yl)quinazoline-6-yl)pyridin-3-yl)-2,4-difluorobenzenesulfonamide

N-(2-(difluoromethoxy)-5-(4-(piperazin-1-yl)quinazolin-6-yl)pyridin-3-yl)-2,4-difluorobenzenesulfonamide trifluoroacetate (150 mg, 0.226 mmol) and tetrahydrofuran (5 mL) were added to the reaction flask. The reaction system was cooled to −78° C., with DIPEA (149 mg, 1.130 mmol), (E)-4-oxopent-2-enoic acid (29 mg, 0.249 mmol) and 50% T₃P ethyl acetate solution (293 mg, 0.452 mmol, 50% wt) added successively. After the addition was completed, the reaction mixture was kept at −78° C. and stirred for 1 h. Upon completion of the reaction, the reaction was quenched using saturated aqueous ammonium chloride solution (50 mL) at −78° C. The reaction mixture was extracted by adding dichloromethane (100 mL×2). The organic phases were combined, washed with saturated brine (100 mL), dried over anhydrous sodium sulfate and filtered. The filtrate was concentrated under reduced pressure. The crude product obtained was purified by Prep-HPLC (Method 2). (E)-N-(2-(difluoromethoxy)-5-(4-(4-(4-oxopent-2-enoyl)piperazin-1-yl)quinazoline-6-(yl)pyridin-3-yl)-2,4-difluorobenzenesulfonamide was obtained, yield: 13.8%; ¹H NMR (400 MHz, DMSO-d₆) δ 11.73 (s, 1H), 8.65 (s, 1H), 8.17-8.03 (m, 1H), 7.99-7.82 (m, 4H), 7.65-7.52 (m, 1H), 7.44 (d, J=15.6 Hz, 2H), 7.36-7.27 (m, 1H), 7.19-7.09 (m, 1H), 6.73 (d, J=16.0 Hz, 1H), 4.02-3.72 (m, 8H), 2.36 (s, 3H); ESI-MS(m/z): 645.0 [M+H]⁺.

### Example 209

### Preparation of (E)-3-fluoro-4-(N-(2-methoxy-5-(4-(4-(4-oxopent-2-enoyl)piperazin-1-yl)quinazolin-4-yl)pyridin-3-yl)sulfamoyl)benzoic acid

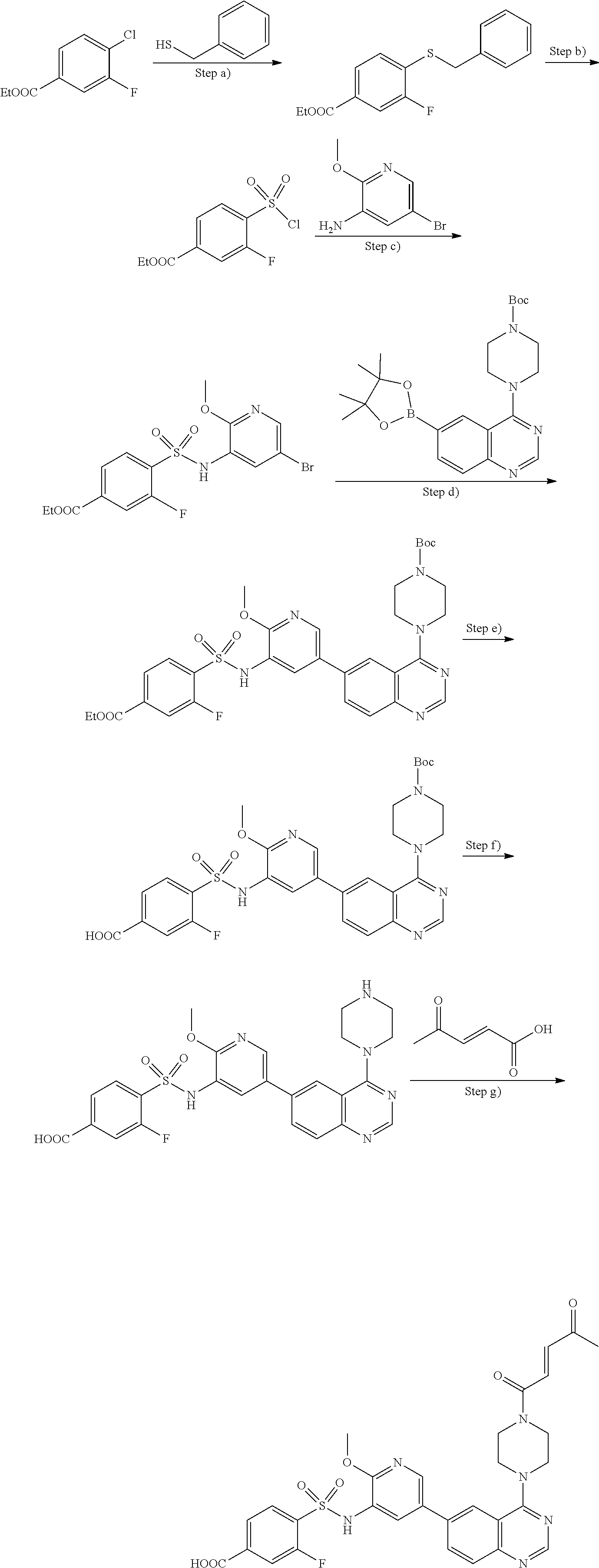

### Step a): Preparation of ethyl 4-(benzylthio)-3-fluorobenzoate

Ethyl 4-chloro-3-fluorobenzoate (1.0 g, 4.936 mmol), cesium carbonate (1.61 g, 4.936 mmol) and DMF (10 mL) were added to the reaction flask, with benzylthiol (736 mg, 5.923 mmol) added. After the addition was completed, the reaction mixture was stirred at room temperature and reacted overnight. Upon completion of the reaction, the reaction was quenched by adding water (50 mL), and the reaction mixture was extracted with dichloromethane (150 mL×2). The organic phases were combined, washed with saturated brine (50 mL), dried over anhydrous sodium sulfate and filtered. The filtrate was concentrated under reduced pressure. The crude product obtained was purified by silica gel column chromatography (Eluent: ethyl acetate/petroleum ether=1/20 to 1/5). Ethyl 4-(benzylthio)-3-fluorobenzoate was obtained, yield: 59.3%; ¹H NMR (400 MHz, DMSO-d₆) δ 7.73 (dd, J₁=8.0, J₂=1.6 Hz, 1H), 7.67-7.57 (m, 2H), 7.43-7.39 (m, 2H), 7.35-7.25 (m, 3H), 4.37 (s, 2H), 4.30 (q, J=7.2 Hz, 2H), 1.31 (t, J=7.2 Hz, 3H).

### Step b): Preparation of ethyl 4-(chlorosulfonyl)-3-fluorobenzoate

Ethyl 4-(benzylthio)-3-fluorobenzoate (400 mg, 1.378 mmol) was added to acetonitrile (8 mL). After that, glacial acetic acid (414 mg, 6.890 mmol) and water (2 mg, 0.138 mmol) were added to the system. After the addition was completed, under the ice bath condition, 1,3-dichloro-5,5-dimethylhydantoin (543 mg, 2.756 mmol) was added. After the addition was completed, the reaction mixture was kept under the ice bath condition and reacted for 2 h. Upon completion of the reaction, the reaction was quenched by adding water (50 mL), and the reaction mixture was extracted with dichloromethane (100 mL×2). The organic phases were combined, washed with saturated brine (50 mL), dried over anhydrous sodium sulfate and filtered. The filtrate was concentrated under reduced pressure. Ethyl 4-(chlorosulfonyl)-3-fluorobenzoate was obtained, yield: 97.8%.

### Step c): Preparation of ethyl 4-(N-(5-bromo-2-methoxypyridin-3-yl)sulfamoyl)-3-fluorobenzoate

Ethyl 4-(chlorosulfonyl)-3-fluorobenzoate (180 mg, 0.675 mmol), 5-bromo-2-methoxypyridin-3-amine (137 mg, 0.675 mmol), pyridine (160 mg, 2.025 mmol), 4-dimethylaminopyridine (8 mg, 0.068 mmol) were added to dichloromethane (100 mL). After the addition was completed, the reaction mixture was kept at room temperature and reacted overnight. Upon completion of the reaction, the reaction was quenched by adding water (50 mL), and the reaction mixture was extracted with dichloromethane (100 mL×2). The organic phases were combined, washed with saturated brine (50 mL), dried over anhydrous sodium sulfate and filtered. The filtrate was concentrated under reduced pressure. The crude product obtained was purified by silica gel column chromatography (Eluent: methanol/dichloromethane=1/100 to 1/30). Ethyl 4-(N-(5-bromo-2-methoxypyridin-3-yl)sulfamoyl)-3-fluorobenzoate was obtained, yield: 45.7%; ESI-MS(m/z): 433.0 [M+H]⁺.

### Step d): Preparation of tert-butyl 4-(6-(5-((4-(ethoxycarbonyl)-2-fluorophenyl)sulfonamido)-6-methoxypyridin-3-yl)quinazolin-4-yl)piperazine-1-carboxylate

Ethyl 4-(N-(5-bromo-2-methoxypyridin-3-yl)sulfamoyl)-3-fluorobenzoate (200 mg, 0.462 mmol), tert-butyl 4-(6-(4,4,5,5-tetramethyl-1,3,2-dioxaborolan-2-yl)quinazolin-4-yl)piperazine-1-carboxylate (203 mg, 0.462 mmol), Pd(dppf)Cl₂ (34 mg, 0.046 mmol) and cesium carbonate (452 mg, 1.386 mmol) were added to dioxane/water mixed solvent (5 mL, v/v=10:1). After the addition was completed, in the presense of protective nitrogen, the reaction mixture was heated to reflux, stirred and reacted for 3 h. Upon completion of the reaction, the reaction was quenched by adding water (30 mL). The reaction mixture was extracted with ethyl acetate (50 mL×2). The organic phases were combined, washed with saturated brine (30 mL), dried over anhydrous sodium sulfate and filtered. The filtrate was concentrated under reduced pressure. The crude product obtained was purified by silica gel column chromatography (Eluent: methanol/dichloromethane=1/100 to 1/30). Tert-butyl 4-(6-(5-((4-(ethoxycarbonyl)-2-fluorophenyl)sulfonamido)-6-methoxypyridin-3-yl)quinazolin-4-yl)piperazine-1-carboxylate was obtained, yield: 59.6%; ESI-MS(m/z): 667.2 [M+H]⁺.

### Step e): Preparation of 4-(N-(5-(4-(4-(tert-butoxycarbonyl)piperazin-1-yl)quinazolin-6-yl)-2-methoxypyridin-3-yl)sulfamoyl)-3-fluorobenzoic acid

Tert-butyl 4-(6-(5-((4-(ethoxycarbonyl)-2-fluorophenyl)sulfonamido)-6-methoxypyridin-3-yl)quinazolin-4-yl)piperazine-1-carboxylate (200 mg, 0.300 mmol) was added to methanol (10 mL), then with sodium hydroxide solution (4 mL, 2M) added. After the addition was completed, the reaction mixture was kept at room temperature and reacted overnight. Upon completion of the reaction, the reaction mixture was concentrated under reduced pressure to remove methanol. The residue was diluted by adding water (30 mL). The impurities were extracted by adding ethyl acetate (50 mL), and the aqueous phase was adjusted to pH=3 with dilute hydrochloric acid solution (2M) to precipitate solids. The solution was filtered and the filter cake was collected and vacuum dried to obtain 4-(N-(5-(4-(4-(tert-butoxycarbonyl)piperazin-1-yl)quinazolin-6-yl)-2-methoxypyridin-3-yl)sulfamoyl)-3-fluorobenzoic acid, yield: 48.3%; ESI-MS(m/z): 639.2 [M+H]⁺.

### Step f): Preparation of 3-fluoro-4-(N-(2-methoxy-5-(4-(piperazin-1-yl)quinazolin-6-yl)pyridin-3-yl)sulfamoyl)benzoic acid trifluoroacetate

4-(N-(5-(4-(4-(tert-butoxycarbonyl)piperazin-1-yl)quinazolin-6-yl)-2-methoxypyridin-3-yl)sulfamoyl)-3-fluorobenzoic acid (60 mg, 0.094 mmol) was dissolved in dichloromethane (4 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. The crude product was slurried with methyl tert-butyl ether (5 mL) to precipitate solid and filtered. 3-Fluoro-4-(N-(2-methoxy-5-(4-(piperazin-1-yl)quinazolin-6-yl)pyridin-3-yl)sulfamoyl)benzoic acid trifluoroacetate was obtained, yield: 91.2%; ESI-MS(m/z): 539.1 [M+H]⁺.

### Step g): Preparation of (E)-3-fluoro-4-(N-(2-methoxy-5-(4-(4-(4-oxopent-2-enoyl)piperazin-1-yl)quinazolin-6-yl)pyridin-3-yl)sulfamoyl)benzoic acid

3-Fluoro-4-(N-(2-methoxy-5-(4-(piperazin-1-yl)quinazolin-6-yl)pyridin-3-yl)sulfamoyl)benzoic acid trifluoroacetate (40 mg, 0.061 mmol) and tetrahydrofuran (2 mL) were added to the reaction flask. The reaction system was cooled to −78° C., with DIPEA (47 mg, 0.366 mmol). (E)-4-oxopent-2-enoic acid (7 mg, 0.061 mmol) and 50% T₃P ethyl acetate solution (78 mg, 0.122 mmol, 50% wt) added successively. After the addition was completed, the reaction mixture was kept at −78° C. and stirred for 1 h. Upon completion of the reaction, the reaction solution was diluted by adding acetonitrile (1 mL) and purified by Prep-HPLC (Method 2). (E)-3-fluoro-4-(N-(2-methoxy-5-(4-(4-(4-oxopent-2-enoyl)piperazin-1-yl)quinazolin-6-yl)pyridin-3-yl)sulfamoyl)benzoic acid was obtained, yield: 10.5%; ¹H NMR (400 MHz, DMSO-4) δ 8.66 (s, 1H), 8.14 (s, 1H), 8.12-8.01 (m, 2H), 7.96-7.87 (m, 2H), 7.85-7.69 (m, 3H), 7.45 (d, J=16.0 Hz, 1H), 6.73 (d, J=16.0 Hz, 1H), 4.02-3.72 (m, 8H), 3.67 (s, 3H), 2.37 (s, 3H); ESI-MS(m/z): 635.0 [M+H]⁺.

### Example 210

### Preparation of (E)-3,5-difluoro-N-(2-methoxy-5-(4-(4-(4-oxopent-2-enoyl)piperazin-1-yl)pyrido[3,2-d]pyrimidin-6-yl)pyridin-3-yl)pyridine-4-sulfonamide

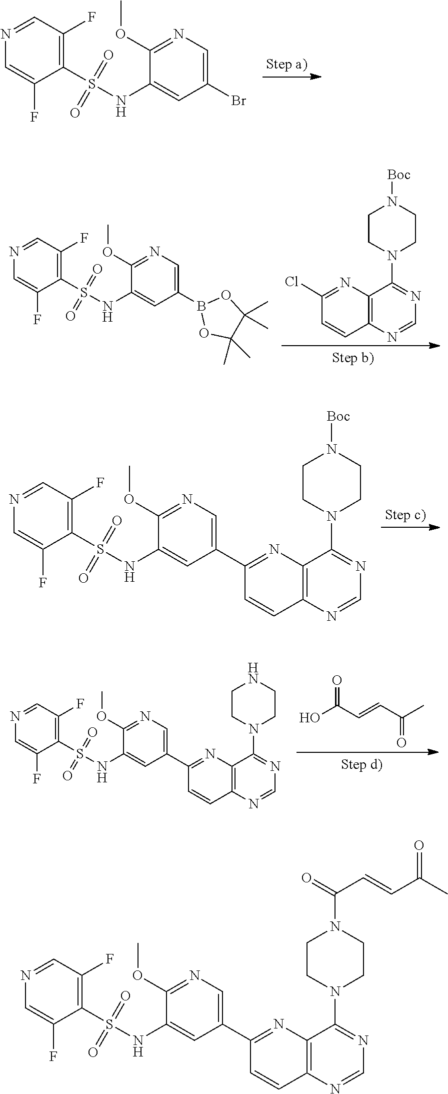

### Step a): Preparation of 3,5-difluoro-N-(2-methoxy-5-(4,4,5,5-tetramethyl-1,3,2-dioxaborolan-2-yl)pyridine-3-yl)pyridine-4-sulfonamide

N-(5-bromo-2-methoxypyridin-3-yl)-3,5-difluoropyridine-4-sulfonamide (100 mg, 0.263 mmol), pinacol diborate (90 mg, 0.354 mmol), Pd(dppf)Cl₂ (19 mg, 0.026 mmol) and potassium acetate (77 mg, 0.789 mmol) were added to 1,4-dioxane (10 mL). After the addition was completed, in the presense of protective nitrogen, the reaction mixture was heated to 90° C. and reacted for 2 h. Upon completion of the reaction, the reaction was quenched by adding water (50 mL). The reaction mixture was extracted with ethyl acetate (100 mL×2). The organic phases were combined, washed with saturated brine (50 mL), dried over anhydrous sodium sulfate and filtered. The filtrate was concentrated under reduced pressure. The crude product obtained was purified by silica gel column chromatography (Eluent: dichloromethane/methanol=100/1 to 50/1), 3,5-Difluoro-N-(2-methoxy-5-(4,4,5,5-tetramethyl-1,3,2-dioxaborolan-2-yl)pyridine-3-yl)pyridine-4-sulfonamide was obtained, yield: 91.2%; ESI-MS(m/z): 428.1 [M+H]⁺.

### Step b): Preparation of tert-butyl 4-(6-(5-((3,5-difluoropyridine)-4-sulfonamido)-6-methoxypyridin-3-yl)pyrido[3,2-d]pyrimidin-4-yl)piperidine-1-carboxylate

3,5-Difluoro-N-(2-methoxy-5-(4,4,5,5-tetramethyl-1,3,2-dioxaborolan-2-yl)pyridine-3-yl)pyridine-4-sulfonamide (100 mg, 0.234 mmol), tert-butyl 4-(6-chloropyrido[3,2-d]pyrimidin-4-yl)piperazine-1-carboxylate (82 mg, 0.234 mmol), Pd(dppf)Cl₂ (17 mg, 0.023 mmol) and cesium carbonate (229 mg, 0.702 mmol) were added to 1,4-dioxane/water mixed solvent (5 mL, v/v=10:1). After the addition was completed, in the presense of protective nitrogen, the reaction mixture was heated to 100° C. and stirred for 2 h. Upon completion of the reaction, the reaction was quenched by adding water (50 mL). The reaction mixture was extracted with ethyl acetate (100 mL×2). The organic phases were combined, washed with saturated brine (50 mL), dried over anhydrous sodium sulfate and filtered. The filtrate was concentrated under reduced pressure. The crude product obtained was purified by silica gel column chromatography (Eluent: dichloromethane/methanol=100/1 to 50/1). Tert-butyl 4-(6-(5-((3,5-di-fluoropyridine)-4-sulfonamido)-6-methoxypyridin-3-yl)pyrido[3,2-d]pyrimidin-4-yl)piperidine-1-carboxylate was obtained, yield: 78.6%; ESI-MS(m/z): 615.2 [M+H]⁻.

### Step c): Preparation of 3,5-difluoro-N-(2-methoxy-5-(4-(piperazin-1-yl)pyrido[3,2-d]pyrimidin-6-yl)pyridin-3-yl)pyridine-4-sulfonamide trifluoroacetate

tert-butyl 4-(6-(5-((3,5-fluoropyridine)-4-sulfonamido)-6-methoxypyridin-3-yl)pyrido[3,2-d]pyrimidin-4-yl)piperidine-1-carboxylate (70 mg, 0.114 mmol) was dissolved in dichloromethane (4 mL) Under the ice bath condition, TFA (I mL) was slowly added dropwise. After the addition was completed, the reaction mixture was stirred at room temperature for 1 h. Upon completion of the reaction, the reaction mixture was concentrated under reduced pressure. The crude product was slurried with methyl tert-butyl ether (5 mL) to precipitate solid and filtered. 3,5-difluoro-N-(2-methoxy-5-(4-(piperazin-1-yl)pyrido[3,2-d]pyrimidin-6-yl)pyridin-3-yl)pyridine-4-sulfonamide trifluoroacetate trifluoroacetate was obtained, yield: 90.6%; ESI-MS (m/z): 515.1 [M+H]⁺.

### Step d): Preparation of (E)-3,5-difluoro-N-(2-methoxy-5-(4-(4-(4-oxopent-2-enoyl) piperazin-1-yl)pyrido[3,2-d]pyrimidin-6-yl)pyridin-3-yl)pyridine-4-sulfonamide

3,5-Difluoro-N-(2-methoxy-5-(4-(piperazin-1-yl)pyrido[3,2-d]pyrimidin-6-yl)pyridin-3-yl)pyridine-4-sulfonamide trifluoroacetate (50 mg, 0.080 mmol) and tetrahydrofuran (2 mL) were added to the reaction flask. The reaction system was cooled to −78° C., with DIPEA (62 mg, 0.480 mmol), (E)-4-oxopent-2-enoic acid (9 mg, 0.080 mmol) and 50% T₃P ethyl acetate solution (102 mg, 0.160 mmol, 50% wt) added successively. After the addition was completed, the reaction mixture was kept at −78° C. and stirred for 1 h. Upon completion of the reaction, the reaction solution was diluted by adding acetonitrile (1 mL) and purified by Prep-HPLC (Method 2). (E)-3,5-difluoro-N-(2-methoxy-5-(4-(4-(4-oxopent-2-enoyl) piperazin-1-yl)pyrido[3,2-d]pyrimidin-6-yl)pyridin-3-yl)pyridine-4-sulfonamide was obtained, yield: 16.1%; ¹H NMR (400 MHZ, DMSO-d₆) δ 11.20 (brs, 1H), 8.73-8.40 (m, 4H), 8.38-8.26 (m, 2H), 8.18 (d, J=9.2 Hz, 1H), 7.47 (d, J=16.0 Hz, 1H), 6.72 (d, J=15.6 Hz, 1H), 4.78-4.22 (m, 4H), 4.01-3.76 (m, 4H), 3.72 (s, 3H), 2.37 (s, 3H); ESI-MS (m/z): 611.0 [M+H]⁺.

Referring to the preparation method of Example 210 and using the corresponding raw materials, the compounds in the following examples were obtained.

| Number | Name | Structure | ¹H NMR and MS |
|---|---|---|---|
| Example 211 | 2,6-difluoro-N- (5-(4-(4-(2- fluoroacryloyl) piperazin-1- yl)pyrido[3,2- d]pyrimidin-6- yl)-2- methoxy- pyridin- 3-yl)benzene- sulfonamide | 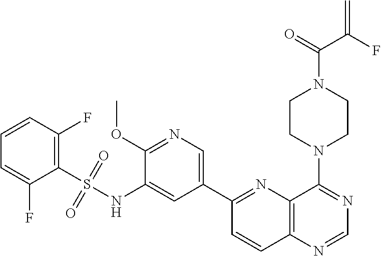 [SMILES: C=C(F)C(=O)N1CCN(c2ncnc3ccc(-c4cnc(OC)c(NS(=O)(=O)c5c(F)cccc5F)c4)nc23)CC1] | ¹H NMR (400 MHz, DMSO-d₆) δ 10.63 (s, 1H), 8.80-8.77 (m, 1H), 8.56 (s, 1H), 8.45-8.39 (m, 2H), 8.21 (d, J = 8.8 Hz, 1H), 7.73-7.65 (m, 1H), 7.25 (t, J = 9.2 Hz, 2H), 5.37-5.20 (m, 2H), 4.50 (s, 4H), 3.82 (s, 4H), 3.72 (s, 3H); ESI-MS (m/z): 586.0 [M + H]⁺. |
| Example 212 | (E)-2,6- difluoro-N-(2- methoxy- 5-(4-(4-(4- oxohex-2- enoyl) piperazin-1-yl) pyrido[3,2-d] pyrimidin-6-yl) pyridin-3-yl) benzene- sulfonamide | 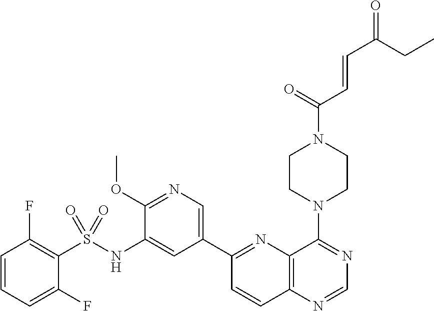 [SMILES: CCC(=O)/C=C/C(=O)N1CCN(c2ncnc3ccc(-c4cnc(OC)c(NS(=O)(=O)c5c(F)cccc5F)c4)nc23)CC1] | ¹H NMR (400 MHz, DMSO-d₆) δ 10.62 (s, 1H), 8.75 (s, 1H), 8.56 (s, 1H), 8.40 (d, J = 8.4 Hz, 2H), 8.20 (d, J = 8.8 Hz, 1H), 7.72-7.65 (m, 1H), 7.24 (t, J = 18.8 Hz, 2H), 6.58 (d, J = 12.0 Hz, 1H), 6.44 (d, J = 15.6 Hz, 1H), 4.47 (s, 4H), 3.74 (s, 2H), 3.73 (s, 3H), 3.65-3.62 (m, 2H), 2.60-2.54 (m, 2H), 0.94 (t, J = 14.4 Hz, 3H); ESI- MS (m/z): 624.0 [M + H]⁺. |
| Example 213 | (E)-2,6- difluoro- N-(5-(4-(4-(4- fluorobut-2- enoyl) piperazin-1- yl)pyrido[3,2-d] pyrimidine-6- yl)-2-methoxy- pyridin-3-yl) benzene- sulfonamide | 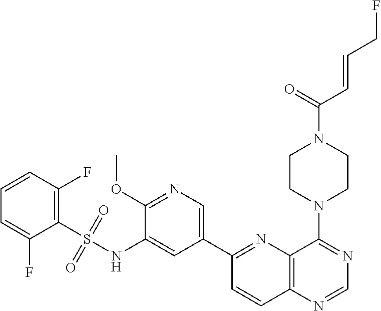 [SMILES: COc1ncc(-c2ccc3ncnc(N4CCN(C(=O)/C=C/CF)CC4)c3n2)cc1NS(=O)(=O)c1c(F)cccc1F] | ¹H NMR (400 MHz, DMSO-d₆) δ 10.64 (s, 1H), 8.61 (d, J = 44.4 Hz, 2H), 8.39-8.33 (m, 2H), 8.19 (d, J = 8.8 Hz, 1H), 7.66-7.57 (m, 1H), 7.19 (t, J = 9.2 Hz, 2H), 6.87-6.71 (m, 2H), 5.22-5.17 (m, 1H), 5.09-5.06 (m, 1H), 4.47 (s, 4H), 3.89-3.76 (m, 4H), 3.73 (s, 3H); ESI-MS (m/z): 600.0 [M + H]⁺. |
| Example 214 | N-(5-(4-(4- acryloyl- piperazin-1-yl) pyrido[3,2-d] pyrimidin-6-yl)- 2-methoxy- pyridin-3-yl)- 2,6-difluoro- benzene- sulfonamide | 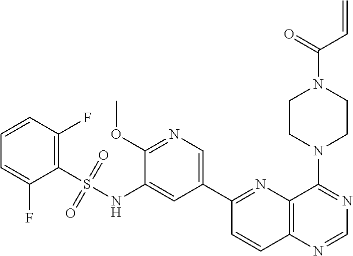 [SMILES: C=CC(=O)N1CCN(c2ncnc3ccc(-c4cnc(OC)c(NS(=O)(=O)c5c(F)cccc5F)c4)nc23)CC1] | ¹H NMR (400 MHz, DMSO-d₆) δ 10.65 (s, 1H), 8.80-8.77 (m, 1H), 8.56 (s, 1H), 8.43-8.39 (m, 2H), 8.20 (d, J = 8.8 Hz, 1H), 7.73-7.64 (m, 1H), 7.25 (t, J = 9.2 Hz, 2H), 6.83 (dd, J₁ = 16.8 Hz, J₂ = 10.4 Hz, 1H), 6.21-6.15 (m, 1H), 5.77-5.72 (m, 1H), 4.47 (s, 4H), 3.88-3.77 (m, 4H), 3.72 (s, 3H); ESI-MS (m/z): 568.0 [M + H]⁺. |
| Example 215 | (E)-2,6-difluoro- N-(2-methoxy- 5-(4-(4-(4- methoxybut-2- enoyl)piperazin- 1-yl)pyridino [3,2-d] pyrimidin-6-yl) pyridin-3-yl) benzene- sulfonamide | 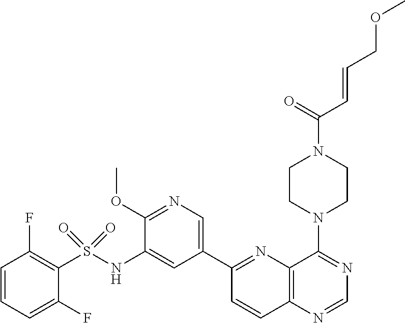 [SMILES: COC/C=C/C(=O)N1CCN(c2ncnc3ccc(-c4cnc(OC)c(NS(=O)(=O)c5c(F)cccc5F)c4)nc23)CC1] | ¹H NMR (400 MHz, DMSO-d₆) δ 10.63 (s, 1H), 8.76 (s, 1H), 8.55 (s, 1H), 8.40 (d, J = 10.0 Hz, 2H), 8.20 (d, J = 8.8 Hz, 1H), 7.74-7.59 (m, 1H), 7.24 (t, J = 9.2 Hz, 2H), 6.71 (m, 2H), 4.45 (m, 4H), 4.08 (m, 2H), 3.82 (m, 4H), 3.73 (s, 3H), 3.29 (s, 3H); ESI-MS (m/z): 612.2 [M + H]⁺. |
| Example 216 | (E)-2,6-difluoro- N-(2-methoxy- 5-(4-(4-(4,4,4- trifluorobut-2- enoyl)piperazin- 1-yl)pyridino [3,2-d] pyrimidin- 6-yl)pyridin-3- yl)benzene- sulfonamide | 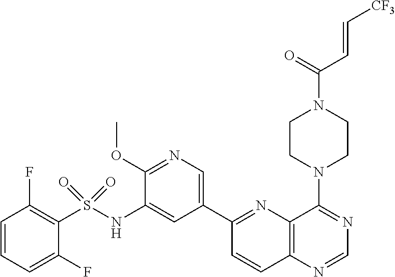 [SMILES: COc1ncc(-c2ccc3ncnc(N4CCN(C(=O)/C=C/C(F)(F)F)CC4)c3n2)cc1NS(=O)(=O)c1c(F)cccc1F] | ¹H NMR (400 MHz, DMSO-d₆) δ 10.62 (s, 1H), 8.73 (s, 1H), 8.56 (s, 1H), 8.38 (d, J = 8.8 Hz, 2H), 8.20 (d, J = 8.8 Hz, 1H), 7.68-7.63 (m, 1H), 7.40 (d, J = 15.6 Hz, 1H), 7.22 (t, J = 18.8 Hz, 2H), 6.87-6.78 (m, 1H), 4.49 (s, 4H), 3.88 (s, 2H), 3.81 (s, 2H), 3.73 (s, 3H); ESI-MS (m/z): 636.0 [M + H]⁺. |
| Example 217 | (E)-2,5-difluoro- N-(2-methoxy- 5-(4-(4-(4- oxopent-2- enoyl) piperazin-1-yl) pyrido[3,2-d] pyrimidin-6-yl) pyridin-3-yl) benzene- sulfonamide | 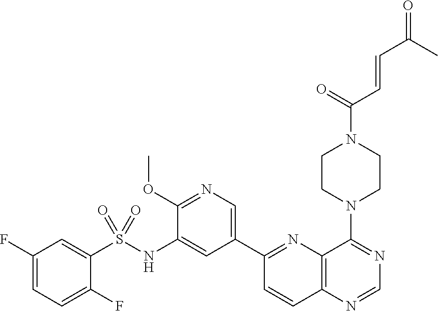 [SMILES: COc1ncc(-c2ccc3ncnc(N4CCN(C(=O)/C=C/C(C)=O)CC4)c3n2)cc1NS(=O)(=O)c1cc(F)ccc1F] | ¹H NMR (400 MHz, DMSO-d₆) δ 10.31 (s, 1H), 8.62 (d, J = 4.8 Hz, 1H), 8.55 (s, 1H), 8.38-8.34 (m, 2H), 8.18 (d, J = 8.8 Hz, 1H), 7.52-7.41 (m, 4H), 6.72 (d, J = 16.0 Hz, 1H), 4.72- 4.26 (m, 4H), 3.95-3.92 (m, 2H), 3.83-3.77 (m, 5H), 2.36 (s, 3H); ESI- MS (m/z): 610.0 [M + H]⁺. |
| Example 218 | (E)-2,3-difluoro- N-(2-methoxy- 5-(4-(4-(4- oxopent-2- enoyl) piperazin-1-yl) pyrido[3,2-d] pyrimidin-6-yl) pyridin-3-yl) benzene- sulfonamide | 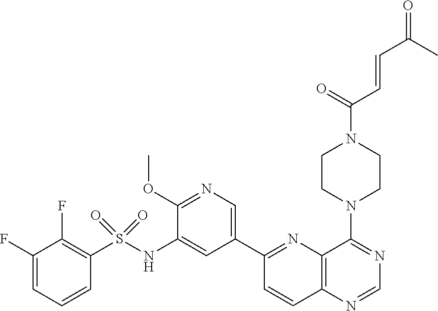 [SMILES: COc1ncc(-c2ccc3ncnc(N4CCN(C(=O)/C=C/C(C)=O)CC4)c3n2)cc1NS(=O)(=O)c1cccc(F)c1F] | ¹H NMR (400 MHz, DMSO-d₆) δ 10.59 (brs, 1H), 8.76 (s, 1H), 8.57 (s, 1H), 8.44-8.36 (m, 2H), 8.21 (d, J = 8.8 Hz, 1H), 7.77-7.66 (m, 1H), 7.54- 7.42 (m, 2H), 7.36-7.27 (m, 1H), 6.72 (d, J = 15.6 Hz, 1H), 4.64-4.37 (m, 4H), 3.98-3.79 (m, 4H), 3.73 (s, 3H), 2.36 (s, 3H); ESI-MS (m/z): 610.0 [M + H]⁺. |
| Example 219 | (E)-2,4-trifluoro- N-(2-methoxy- 5-(4-(4-(4- oxopent-2- enoyl) piperazin-1-yl) pyrido[3,2-d] pyrimidin-6-yl) pyridin-3-yl) benzene- sulfonamide | 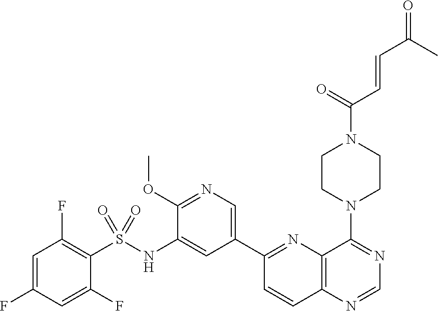 [SMILES: COc1ncc(-c2ccc3ncnc(N4CCN(C(=O)/C=C/C(C)=O)CC4)c3n2)cc1NS(=O)(=O)c1c(F)cc(F)cc1F] | ¹H NMR (400 MHz, DMSO-d₆) δ 10.67 (s, 1H), 8.61 (s, 1H), 8.55 (s, 1H), 8.36-8.32 (m, 2H), 8.18 (d, J = 8.8 Hz, 1H), 7.46 (d, J = 16.0 Hz, 1H), 7.28 (t, J = 9.6, 2H), 6.72 (d, J = 16.0 Hz, 1H), 4.66-4.35 (m, 4H), 3.96-3.90 (m, 2H), 3.83-3.79 (m, 2H), 3.77 (s, 3H), 2.36 (s, 3H); ESI-MS (m/z): 628.0 [M + H]⁺. |
| Example 220 | (E)-3-fluoro-N- (2-methoxy-5- (4-(4-(4- oxopent- 2-enoyl) piperazin-1-yl) pyrido[3,2-d] pyrimidin-6-yl) pyridin-3-yl) pyridine-4- sulfonamide | 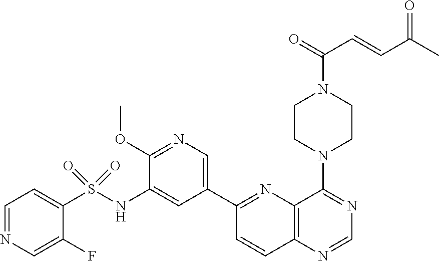 [SMILES: COc1ncc(-c2ccc3ncnc(N4CCN(C(=O)/C=C/C(C)=O)CC4)c3n2)cc1NS(=O)(=O)c1ccncc1F] | ¹H NMR (400 MHz, DMSO-d₆) δ 10.83 (brs, 1H), 8.80 (s, 1H), 8.71 (s, 1H), 8.60-8.52 (m, 2H), 8.42-8.31 (m, 2H), 8.20 (d, J = 9.2 Hz, 1H), 7.69- 7.64 (m, 1H), 7.47 (d, J = 15.6 Hz, 1H), 6.72 (d, J = 16.0 Hz, 1H), 4.69- 4.30 (m, 4H), 3.98-3.77 (m, 4H), 3.71 (s, 3H), 2.36 (s, 3H); ESI-MS (m/z): 593.0 [M + H]⁺. |
| Example 221 | (E)-2-fluoro-N- (2-methoxy-5- (4-(4-(4- oxopent-2- enoyl)piperazin- 1-yl)pyrido[3,2- d]pyrimidin-6- yl)pyridin-3- yl)-4-(trifluoro- methyl)benzene- sulfonamide | 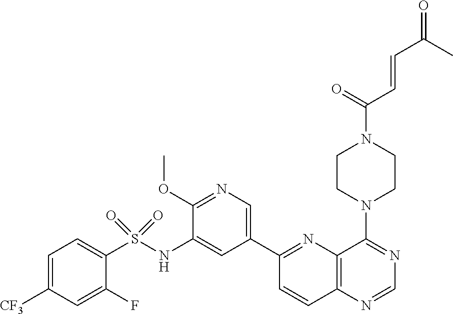 [SMILES: COc1ncc(-c2ccc3ncnc(N4CCN(C(=O)/C=C/C(C)=O)CC4)c3n2)cc1NS(=O)(=O)c1ccc(C(F)(F)F)cc1F] | ¹H NMR (400 MHz, DMSO-d₆) δ 10.59 (s, 1H), 8.78-8.73 (m, 1H), 8.56 (s, 1H), 8.43-8.38 (m, 2H), 8.20 (d, J = 8.8 Hz, 1H), 7.98 (d, J = 10.0 Hz, 1H), 7.92 (t, J = 7.6 Hz, 1H), 7.73- 7.69 (m, 1H), 7.46 (d, J = 15.6 Hz, 1H), 6.72 (d, J = 15.6 Hz, 1H), 4.50 (s, 4H), 3.96-3.90 (m, 2H), 3.85-3.80 (m, 2H), 3.69 (s, 3H), 2.35 (s, 3H); ESI-MS (m/z): 660.0 [M + H]⁺. |
| Example 222 | (E)-N-(2- methoxy-5- (4-(4- (4-oxopent-2- enoyl)piperazin- 1-yl)pyrido[3,2- d]pyrimidin-6- yl)pyridin-3-yl)- 2,4-dimethyl- thiazole-5- sulfonamide | 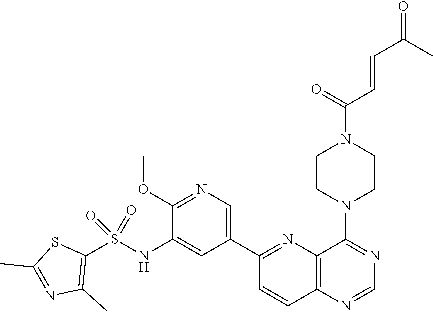 [SMILES: COc1ncc(-c2ccc3ncnc(N4CCN(C(=O)/C=C/C(C)=O)CC4)c3n2)cc1NS(=O)(=O)c1sc(C)nc1C] | ¹H NMR (400 MHz, DMSO-d₆) δ 10.41 (s, 1H), 8.66 (s, 1H), 8.56 (s, 1H), 8.39-8.36 (m, 2H), 8.20 (d, J = 8.8 Hz, 1H), 7.47 (d, J = 16.0 Hz, 1H), 6.72 (d, J = 15.6 Hz, 1H), 4.80- 4.20 (m, 4H), 3.95-3.93 (m, 2H), 3.84-3.80 (m, 5H), 2.53 (s, 3H), 2.38 (s, 3H), 2.36 (s, 3H); ESI-MS (m/z): 609.0 [M + H]⁺. |
| Example 223 | (E)-N-(2- methoxy-5- (4-(4- (4-oxopent-2- enoyl)piperazin- 1-yl)pyrido[3,2- d]pyrimidin-6- yl)pyridin-3-yl)- 4-methyl- pyridine-3- sulfonamide | 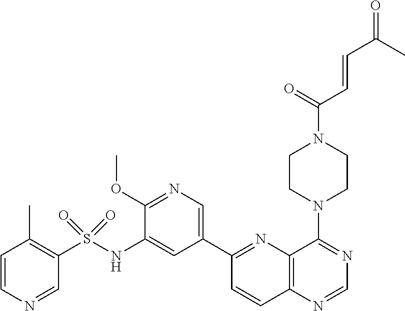 | ¹H NMR (400 MHz, DMSO-d₆) δ 10.41 (s, 1H), 8.71 (s, 1H), 8.64 (s, 1H), 8.59-8.51 (m, 2H), 8.41-8.34 (m, 2H), 8.19 (d, J = 8.8 Hz, 1H), 7.49 (d, J = 15.6 Hz, 1H), 7.42 (d, J = 5.2 Hz, 1H), 6.73 (d, J = 15.6 Hz, 1H), 4.75- 4.28 (m, 4H), 4.01-3.80 (m, 4H), 3.73 (s, 3H), 2.67 (s, 3H), 2.35 (s, 3H); ESI-MS (m/z): 660.0 [M + H]⁺. |
| Example 224 | (E)-3,5- dichloro-N-(2- methoxy-5-(4- (4-(4-oxopent- 2-enoyl) piperazin-1-yl) pyrido[3,2-d] pyrimidin-6- yl)pyridin-3-yl) pyridine-4- sulfonamide | 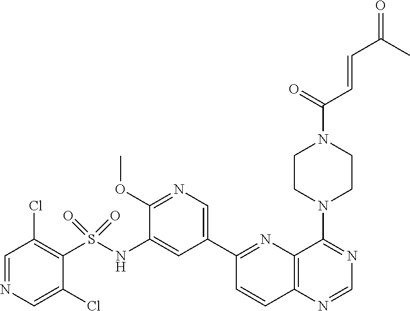 [SMILES: COc1ncc(-c2ccc3ncnc(N4CCN(C(=O)/C=C/C(C)=O)CC4)c3n2)cc1NS(=O)(=O)c1c(Cl)cncc1Cl] | ¹H NMR (400 MHz, DMSO-d₆) δ 10.98 (brs, 1H), 8.73 (s, 2H), 8.69- 8.62 (m, 1H), 8.56 (s, 1H), 8.36 (d, J = 8.8 Hz, 1H), 8.28 (d, J = 2.4 Hz, 1H), 8.20 (d, J = 8.8 Hz, 1H), 7.47 (d, J = 16.0 Hz, 1H), 6.73 (d, J = 15.6 Hz, 1H), 4.76-4.24 (m, 4H), 4.01-3.77 (m, 4H), 3.65 (s, 3H), 2.36 (s, 3H); ESI-MS (m/z): 643.0 [M + H]⁺. |
| Example 225 | (E)-2-chloro-6- fluoro-N-(2- methoxy-5-(4- (4-(4- oxopent-2- enoyl)piperazin- 1-yl)pyrido[3,2- d]pyrimidin-6- yl)pyridin-3-yl) benzene- sulfonamide | 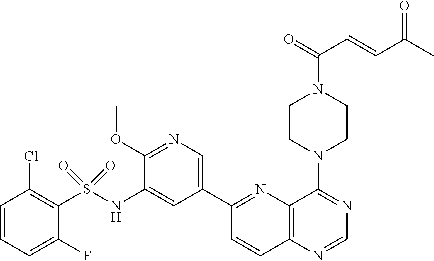 [SMILES: COc1ncc(-c2ccc3ncnc(N4CCN(C(=O)/C=C/C(C)=O)CC4)c3n2)cc1NS(=O)(=O)c1c(F)cccc1Cl] | ¹H NMR (400 MHz, DMSO-d₆) δ 10.27 (brs, 1H), 8.57-8.49 (m, 2H), 8.34-8.25 (m, 2H), 8.18 (d, J = 8.8 Hz, 1H), 7.55-7.43 (m, 2H), 7.38 (d, J = 8.0 Hz, 1H), 7.33-7.24 (m, 1H), 6.72 (d, J = 16.0 Hz, 1H), 4.68-4.25 (m, 4H), 4.00-3.88 (m, 2H), 3.86-3.78 (m, 2H), 3.75 (s, 3H), 2.36 (s, 3H); ESI-MS (m/z): 626.0 [M + H]⁺. |
| Example 226 | (E)-2,4-difluoro- N-(5-(4-(4-(4- oxopent-2- enoyl)piperazin- 1-yl)pyrido[3,2- d]pyrimidin-6- yl)-2-(trifluoro- methoxy) pyridin- 3-yl)benzene- sulfonamide | 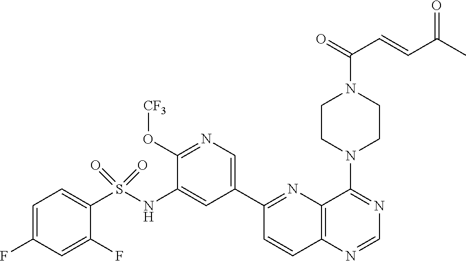 [SMILES: CC(=O)/C=C/C(=O)N1CCN(c2ncnc3ccc(-c4cnc(OC(F)(F)F)c(NS(=O)(=O)c5ccc(F)cc5F)c4)nc23)CC1] | ¹H NMR (400 MHz, DMSO-d₆) δ 11.02 (br s, 1H), 8.76-8.50 (m, 3H), 8.48-8.35 (m, 1H), 8.24 (d, J = 8.8 Hz, 1H), 7.84-7.72 (m, 1H), 7.50-7.37 (m, 2H), 7.20-7.09 (m, 1H), 6.72 (d, J = 16.0 Hz, 1H), 4.70-4.35 (m, 4H), 4.00-3.90 (m, 2H), 3.86-3.77 (m, 2H), 2.35 (s, 3H); ESI-MS (m/z): 664.0 [M + H]⁺. |
| Example 227 | (E)-N-(2- ethoxy- 5-(4-(4-(4- oxopent-2- enoyl) piperazin-1-yl) pyrido[3,2-d] pyrimidin-6-yl) pyridin-3-yl)-2- fluorobenzene- sulfonamide | 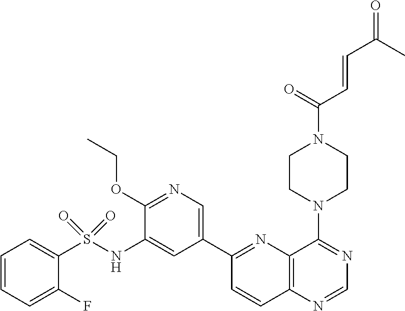 [SMILES: CCOc1ncc(-c2ccc3ncnc(N4CCN(C(=O)/C=C/C(C)=O)CC4)c3n2)cc1NS(=O)(=O)c1ccccc1F] | ¹H NMR (400 MHz, DMSO-d₆) δ 8.66-8.51 (m, 2H), 8.39-8.31 (m, 2H), 8.18 (d, J = 8.8 Hz, 1H), 7.76-7.67 (m, 1H), 7.65-7.55 (m, 1H), 7.47 (d, J = 16.0 Hz, 1H), 7.36 (t, J = 9.2 Hz, 1H), 7.25 (t, J = 7.6 Hz, 1H), 6.72 (d, J = 15.6 Hz, 1H), 4.51 (d, J = 39.8 Hz, 4H), 4.25-4.21 (m, 2H), 3.88 (d, J = 47.6 Hz, 4H), 2.36 (s, 3H), 1.18 (d, J = 6.8 Hz, 3H); ESI-MS (m/z): 606.0 [M + H]⁺. |
| Example 228 | (R,E)-2-fluoro- N-(2-methoxy- 5-(4-(3-methyl- 4-(4-oxopent-2- enoyl)piperazin- 1-yl)pyrido[3,2- d]pyrimidin-6- yl)pyridin-3- yl)benzene- sulfonamide | 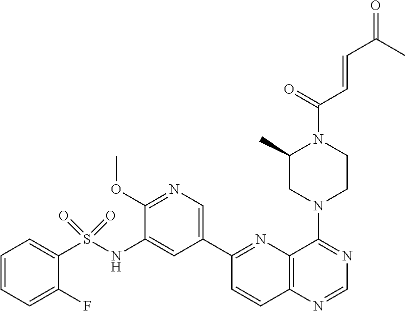 [SMILES: COc1ncc(-c2ccc3ncnc(N4CCN(C(=O)/C=C/C(C)=O)[C@H](C)C4)c3n2)cc1NS(=O)(=O)c1ccccc1F] | ¹H NMR (400 MHz, DMSO-d₆) δ 10.32 (s, 1H), 8.79-8.77 (m, 1H), 8.55 (s, 1H), 8.43-8.38 (m, 2H), 8.20 (d, J = 8.8 Hz, 1H), 7.74-7.66 (m, 2H), 7.48-7.40 (m, 2H), 7.31 (t, J = 8.0 Hz, 1H), 6.77-6.66 (m, 1H), 5.42 (s, 2H), 4.84-4.57 (m, 1H), 4.43-4.13 (m, 1H), 3.83-3.76 (m, 1H), 3.73 (s, 3H), 3.57- 3.37 (m, 2H), 2.36-2.33 (m, 3H), 1.27-1.14 (m, 3H); ESI-MS (m/z): 606.0 [M + H]⁺. |
| Example 229 | (E)-2-fluoro-N- (2-methoxy-5- (4-(4-(4- oxopent-2- enoyl)piperazin- 1-yl)pyrido[3,2- d]pyrimidin-6- yl)pyridin-3-yl)- 6-methyl- benzene- sulfonamide | 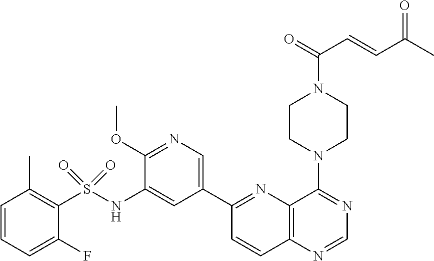 [SMILES: COc1ncc(-c2ccc3ncnc(N4CCN(C(=O)/C=C/C(C)=O)CC4)c3n2)cc1NS(=O)(=O)c1c(C)cccc1F] | ¹H NMR (400 MHz, DMSO-d₆) δ 10.22 (s, 1H), 8.78 (d, J = 2.4 Hz, 1H), 8.57 (s, 1H), 8.45 (d, J = 2.0 Hz, 1H), 8.41 (d, J = 8.8 Hz, 1H), 8.22 (d, J = 8.8 Hz, 1H), 7.56-7.42 (m, 2H), 7.25-7.12 (m, 2H), 6.73 (d, J = 15.6 Hz, 1H), 4.51 (br s, 4H), 3.96-3.88 (m, 2H), 3.86-3.79 (m, 2H), 3.70 (s, 3H), 2.50 (s, 3H), 2.36 (s, 3H); ESI- MS (m/z): 606.0 [M + H]⁺. |

### Example 230

### Preparation of (E)-N-(2-methoxy-5-(4-(1-(4-oxopent-2-enoyl)piperidin-4-yl)pyrido[3,2-d]pyrimidin-6-yl)pyridin-3-yl)-2,4-dimethylthiazol-5-sulfonamide

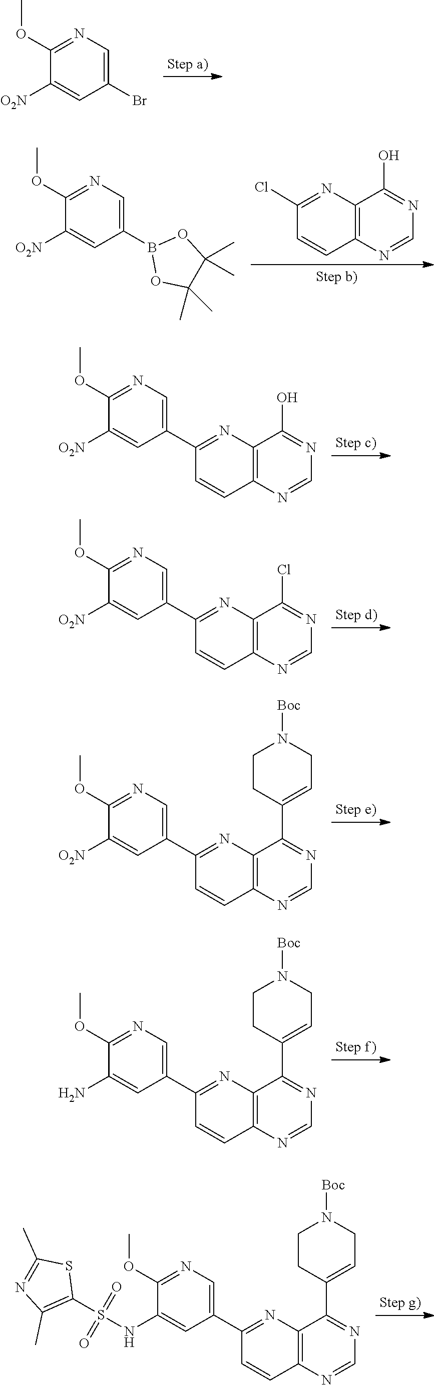

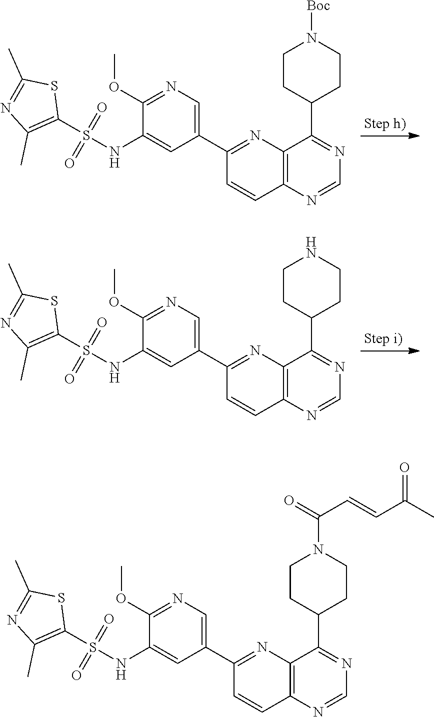 [SMILES: COc1ncc(-c2ccc3ncnc(C4CCN(C(=O)OC(C)(C)C)CC4)c3n2)cc1NS(=O)(=O)c1sc(C)nc1C ; COc1ncc(-c2ccc3ncnc(C4CCNCC4)c3n2)cc1NS(=O)(=O)c1sc(C)nc1C ; COc1ncc(-c2ccc3ncnc(C4CCN(C(=O)/C=C/C(C)=O)CC4)c3n2)cc1NS(=O)(=O)c1sc(C)nc1C]

### Step a): Preparation of 2-methoxy-3-nitro-5-(4,4,5,5-tetramethyl-1,3,2-dioxaborolan-2-yl)pyridine

5-Bromo-2-methoxy-3-nitropyridine (2.0 g, 8.583 mmol), pinacol diborate (2.6 g, 10.300 mmol), Pd(dppf)Cl₂ (628 mg, 0.858 mmol) and potassium acetate (2.5 g, 25.749 mmol) were added to 1,4-dioxane (30 mL). After the addition was completed, in the presense of protective nitrogen, the reaction mixture was heated to 110° C. and reacted for 2 h. Upon completion of the reaction, the reaction was quenched by adding water (50 mL). The reaction mixture was extracted with ethyl acetate (150 mL×2). The organic phases were combined, washed with saturated brine (50 mL), dried over anhydrous sodium sulfate and filtered. The filtrate was concentrated under reduced pressure. The crude product obtained was purified by silica gel column chromatography (Eluent: petroleum ether/ethyl acetate=5/1 to 1/1), 2-Methoxy-3-nitro-5-(4,4,5,5-tetramethyl-1,3,2-dioxaborolan-2-yl)pyridine was obtained, yield: 91.7%; ESI-MS(m/z): 281.3 [M+H]⁺.

### Step b): Preparation of 6-(6-methoxy-5-nitropyridin-3-yl)pyrido[3,2-d]pyrimidin-4-ol

2-Methoxy-3-nitro-5-(4,4,5,5-tetramethyl-1,3,2-dioxaborolan-2-yl)pyridine (1.2 g, 4.284 mmol), 6-chloropyrido[3,2-d]pyrimidin-4-ol (856 mg, 4.712 mmol), Pd(dppf)Cl₂ (313 mg, 0.428 mmol) and cesium carbonate (4.2 g, 12.852 mmol) were added to dioxane/water mixed solvent (50 mL, v/v=9:1). After the addition was completed, in the presense of protective nitrogen, the reaction mixture was heated to 100° C., stirred and reacted for 3 h. Upon completion of the reaction, the reaction was quenched by adding water (30 mL). The reaction mixture was extracted with ethyl acetate (50 mL×2). The organic phases were combined, washed with saturated brine (30 mL), dried over anhydrous sodium sulfate and filtered. The filtrate was concentrated under reduced pressure. The crude product obtained was purified by silica gel column chromatography (Eluent: methanol/dichloromethane=1/100 to 1/30), 6-(6-Methoxy-5-nitropyridin-3-yl)pyrido[3,2-d]pyrimidin-4-ol was obtained, yield: 62.5%; ESI-MS(m/z): 300.3 [M+H]⁺.

### Step c): Preparation of 4-chloro-6-(6-methoxy-5-nitropyridin-3-yl)pyrido[3,2-d]pyrimidine

6-(6-Methoxy-5-nitropyridin-3-yl)pyrido[3,2-d]pyrimidin-4-ol (850 mg, 2.841 mmol) was added to thionyl chloride (30 mL), then with DMF (21 mg, 0.284 mmol) added. After the addition was completed, the reaction mixture was kept at 100° C. and reacted overnight. Upon completion of the reaction, the reaction solution was concentrated under reduced pressure, under the ice bath condition, adjusted to pH=7-8 with saturated aqueous sodium bicarbonate solution and extracted with dichloromethane (100 mL×3). The organic phases were combined, washed with saturated brine (30 mL), dried over anhydrous sodium sulfate and filtered. The filtrate was concentrated under reduced pressure. The crude product obtained was purified by silica gel column chromatography (Eluent: methanol/dichloromethane=l/100 to 1/30), 4-Chloro-6-(6-methoxy-5-nitropyridin-3-yl)pyrido[3,2-d]pyrimidine was obtained, yield: 51.0%; ESI-MS(m/z): 318.3 [M+H]r.

### Step d): Preparation of tert-butyl 4-(6-(6-methoxy-5-nitropyridin-3-yl)pyrido[3,2-d]pyrimidin-4-yl)-3,6-dihydropyridine-1 (2H)-carboxylate

4-Chloro-6-(6-methoxy-5-nitropyridin-3-yl)pyrido[3,2-d]pyrimidine (460 mg, 1.448 mmol), tert-butyl 4-(4,4,5,5-tetramethyl-1,3,2-dioxaborolan-2-yl)-3,6-dihydropyridine-1 (2H)-carboxylate (537 mg, 1.738 mmol) and Pd(dppf)Cl₂ (106 mg, 0.145 mmol) were added to dioxane (10 mL), in the presense of protective nitrogen, cesium carbonate (1.4 g, 4.344 mmol) was dissolved in water (2 mL) and slowly added to the reaction solution. After the addition was completed, the reaction mixture was stirred at room temperature and reacted for 6 h. Upon completion of the reaction, the reaction mixture was diluted by adding water (30 mL) and extracted with ethyl acetate (50 mL×2). The organic phases were combined, washed with saturated brine (30 mL), dried over anhydrous sodium sulfate and filtered. The filtrate was concentrated under reduced pressure. The crude product obtained was purified by silica gel column chromatography (Eluent: methanol/dichloromethane=1/100 to 1/10).

Tert-butyl 4-(6-(6-methoxy-5-nitropyridin-3-yl)pyrido[3,2-d]pyrimidin-4-yl)-3,6-dihydropyridine-1 (2H)-carboxylate was obtained, yield: 89.2%; ESI-MS(m/z): 465.1 [M+H]⁺.

### Step e): Preparation of tert-butyl 4-(6-(5-amino-6-methoxypyridin-3-yl)pyrido[3,2-d]pyrimidin-4-yl)-3,6-dihydropyridine-1 (2H)-carboxylate

Tert-butyl 4-(6-(6-methoxy-5-nitropyridin-3-yl)pyrido[3,2-d]pyrimidin-4-yl)-3,6-dihydropyridine-1 (2H)-carboxylate (600 mg, 1.292 mmol), iron powder (720 mg, 12.920 mmol) and ammonium chloride (69 mg, 1.292 mmol) were added to methanol and water mixed solvent (25 mL, v/v=4:1). After the addition was completed, the reaction mixture was heated to 60° C., stirred and reacted for 4 h. Upon completion of the reaction, the reaction mixture was filtered. The filtrate was diluted by adding water (40 mL), extracted with dichloromethane (100 mL×3). The organic phases were combined, washed with saturated brine (50 mL), dried over anhydrous sodium sulfate and filtered. The filtrate was concentrated under reduced pressure. The crude product obtained was purified by silica gel column chromatography (Eluent: methanol/dichloromethane=1/100 to 1/10). Tert-butyl 4-(6-(5-amino-6-methoxypyridin-3-yl)pyrido[3,2-d]pyrimidin-4-yl)-3,6-dihydropyridine-1 (2H)carboxylate was obtained, yield: 82.0%; ESI-MS(m/z): 435.3 [M+H]⁺.

### Step f): Preparation of tert-butyl 4-(6-(5-((2,4-dimethylthiazole)-5-sulfonamido)-6-methoxypyridin-3-ylpyrido[3,2-d]pyrimidin-4-yl)-3,6-dihydropyridine-1 (2H)-carboxylate

Tert-butyl 4-(6-(5-amino-6-methoxypyridin-3-yl)pyrido[3,2-d]-pyrimidin-4-yl)-3,6-dihydropyridine-1 (2H)-carboxylate (430 mg, 0.990 mmol) was dissolved in pyridine (10 mL) and 2,4-dimethylthiazole-5-sulfonyl chloride (419 mg, 1.980 mmol) was added to the mixture. The reaction mixture was kept at room temperature and reacted for 4 h. Upon completion of the reaction, the reaction was quenched by adding water (100 mL). The reaction mixture was extracted with ethyl acetate (100 mL×2). The organic phases were combined, washed with saturated brine (100 mL), dried over anhydrous sodium sulfate and filtered. The filtrate was concentrated under reduced pressure. The crude product obtained was purified by silica gel column chromatography (Eluent: dichloromethane/methanol=20/1 to 10/1). Tert-butyl 4-(6-(5-((2,4-dimethylthiazole)-5-sulfonamido)-6-methoxypyridin-3-yl)pyrido[3,2-d]pyrimidin-4-yl)-3,6-dihydropyridine-1 (2H)-carboxylate was obtained, yield: 38.1%; ESI-MS(m/z): 610.3 [M+H]⁺.

### Step g): Preparation of tert-butyl 4-(6-(5-((2,4-dimethylthiazole)-5-sulfonamido)-6-methoxypyridin-3-yl)pyrido[3,2-d]pyrimidin-4-yl)piperazine-1-carboxylate

Tert-butyl 4-(6-(5-((2,4-dimethylthiazole)-5-sulfonamido)-6-methoxypyridin-3-yl)pyrido[3,2-d]pyrimidin-4-yl)-3,6-dihydropyridine-1 (2H)-carboxylate (180 mg, 0.295 mmol), 10% Pd/C (100 mg), glacial acetic acid (18 mg, 0.295 mmol) and methanol (10 mL) were added to the reaction flask. Under hydrogen atmosphere, the reaction mixture was kept at 30° C. and reacted for 6 h. Upon completion of the reaction, the reaction mixture was filtered. The filtrate was concentrated under reduced pressure. The crude product obtained was purified by silica gel column chromatography (Eluent: dichloromethane/methanol=20/1 to 10/1). Tert-butyl 4-(6-(5-((2,4-dimethylthiazole)-5-sulfonamido)-6-methoxypyridin-3-yl)pyrido[3,2-d]pyrimidin-4-yl)piperazine-1-carboxylate was obtained, yield: 55.2%; ESI-MS(m/z): 612.1 [M+H]⁺.

### Step h): Preparation of N-(2-methoxy-5-(4-(piperidin-4-yl)pyrido[3,2-d]pyrimidin-6-yl)pyridin-3-yl)-2,4-dimethylthiazol-5-sulfonamide trifluoroacetate

Tert-butyl 4-(6-(5-((2,4-Dimethylthiazole)-5-sulfonamido)-6-methoxypyridin-3-yl)pyrido[3,2-d]pyrimidin-4-yl)piperazine-1-carboxylate (100 mg, 0.163 mmol) was dissolved in dichloromethane (4 mL). Under the ice bath condition, TFA (1 mL) was slowly added dropwise. After the addition was completed, the reaction mixture was stirred at room temperature for 1 h. Upon completion of the reaction, the reaction mixture was concentrated under reduced pressure. N-(2-methoxy-5-(4-(piperidin-4-yl)pyrido[3,2-d]pyrimidin-6-yl)pyridin-3-yl)-2,4-dimethylthiazol-5-sulfonamide trifluoroacetate was obtained; ESI-MS(m/z): 512.5 [M+H]⁺.

### Step i): Preparation of (E)-N-(2-methoxy-5-(4-(1-(4-oxopent-2-enoyl)piperidin-4-yl)pyrido[3,2-d]pyrimidin-6-yl)pyridin-3-yl)-2,4-dimethylthiazol-5-sulfonamide

N-(2-methoxy-5-(4-(piperidin-4-yl)pyrido[3,2-d]pyrimidin-6-yl)pyridin-3-yl)-2,4-dimethylthiazol-5-sulfonamide trifluoroacetate (100 mg, 0.160 mmol) and tetrahydrofuran (4 mL) were added to the reaction flask. The reaction system was cooled to −78° C., with DIPEA (103 mg, 0.800 mmol), (E)-4-oxopent-2-enoic acid (20 mg, 0.176 mmol) and 50% T₃P ethyl acetate solution (204 mg, 0.320 mmol, 50% wt) added successively. After the addition was completed, the reaction mixture was kept at −78° C. and stirred for 1 h. Upon completion of the reaction, the reaction was quenched using saturated aqueous ammonium chloride solution (50 mL) at −78° C. The reaction mixture was extracted by adding dichloromethane (100 mL×2). The organic phases were combined, washed with saturated brine (100 mL), dried over anhydrous sodium sulfate and filtered. The filtrate was concentrated under reduced pressure. The crude product obtained was purified by Prep-HPLC (Method 2). (E)-N-(2-methoxy-5-(4-(1-(4-oxopent-2-enoyl)piperidin-4-yl)pyrido[3,2-d]pyrimidin-6-yl)pyridin-3-yl)-2,4-dimethylthiazol-5-sulfonamide was obtained, yield: 17.2%; ¹H NMR (400 MHz, DMSO-d₆) δ 10.49 (brs, 1H), 9.27 (s, 1H), 8.89 (s, 1H), 8.68-8.58 (m, 2H), 8.47 (d, J=8.8 Hz, 1H), 7.50 (d, J=16.0 Hz, 1H), 6.70 (d, J=15.6 Hz, 1H), 4.64 (d, J=12.8 Hz, 1H), 4.57-4.46 (m, 1H), 4.30 (d, J=13.6 Hz, 1H), 3.84 (s, 3H), 3.50-3.38 (m, 1H), 3.12-2.98 (m, 1H), 2.55 (s, 3H), 2.42 (s, 3H), 2.37 (s, 3H), 2.17-2.05 (m, 2H), 1.99-1.75 (m, 2H); ESI-MS(m/z): 608.0 [M+H]⁺.

Referring to the preparation method of Example 230 and using the corresponding raw materials, the compounds in the following examples were obtained.

| Number | Name | Structure | ¹H NMR and MS |
|---|---|---|---|
| Example 231 | (E)-2,4,6- trifluoro-N-(2- methoxy-5-(4- (1-(4-oxopent- 2-enoyl) piperidin-4- yl)pyrido[3,2- d]pyrimidin- 6-yl)pyridin-3- yl)benzene- sulfonamide | 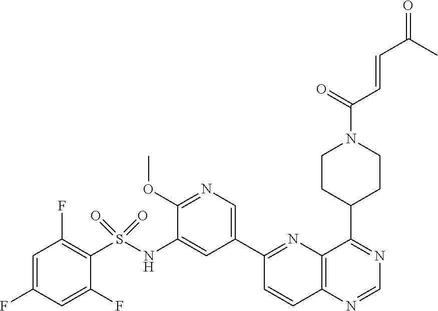 [SMILES: COc1ncc(-c2ccc3ncnc(C4CCN(C(=O)/C=C/C(C)=O)CC4)c3n2)cc1NS(=O)(=O)c1c(F)cc(F)cc1F] | ¹H NMR (400 MHz, DMSO-d₆) δ 10.74 (brs, 1H), 9.25 (s, 1H), 8.81- 8.60 (m, 1H), 8.60-8.32 (m, 3H), 7.50 (d, J = 15.6 Hz, 1H), 7.40-7.04 (m, 2H), 6.70 (d, J = 15.6 Hz, 1H), 4.66- 4.58 (m, 1H), 4.54-4.48 (m, 1H), 4.29-4.25 (m, 1H), 3.80 (s, 3H), 3.43 (t, J = 13.2 Hz, 1H), 3.05-2.97 (m, 1H), 2.37 (s, 3H), 2.12-2.03 (m, 2H), 1.93-1.76 (m, 2H); ESI-MS (m/z): 627.0 [M + H]⁺. |
| Example 232 | (E)-2-fluoro-N- (2-methoxy-5- (4-(1-(4- oxopent-2- enoyl)piperidin- 4-yl)pyrido[3,2- d]pyrimidin-6- yl)pyridin-3- yl)benzene- sulfonamide | 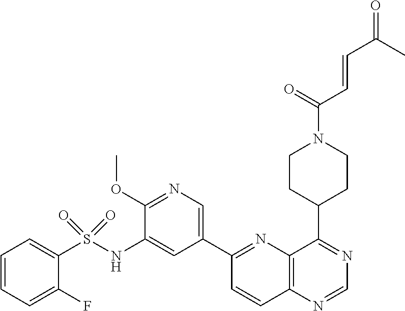 [SMILES: COc1ncc(-c2ccc3ncnc(C4CCN(C(=O)/C=C/C(C)=O)CC4)c3n2)cc1NS(=O)(=O)c1ccccc1F] | ¹H NMR (400 MHz, DMSO-d₆) δ 10.27 (brs, 1H), 9.27 (s, 1H), 8.81 (s, 1H), 8.65-8.35 (m, 3H), 7.76 (t, J = 7.6 Hz, 1H), 7.70-7.56 (m, 1H), 7.51 (d, J = 15.6 Hz, 1H), 7.39 (t, J = 9.6 Hz, 1H), 7.28 (t, J = 7.6 Hz, 1H), 6.70 (d, J = 16.0 Hz, 1H), 4.64 (d, J = 13.2 Hz, 1H), 4.48 (t, J = 11.6 Hz, 1H), 4.29 (d, J = 13.6 Hz, 1H), 3.77 (s, 3H), 3.42 (t, J = 12.8 Hz, 1H), 3.02 (t, J = 12.8 Hz, 1H), 2.37 (s, 3H), 2.16- 2.01 (m, 2H), 2.00-1.88 (m, 1H), 1.85-1.72 (m, 1H); ESI-MS (m/z): 591.2 [M + H]⁺. |
| Example 233 | (E)-2,6- difluoro-N-(2- methoxy-5-(4- (1-(4-oxopent- 2-enoyl) piperidin-4-yl) pyrido[3,2-d] pyrimidin-6- yl)pyridin-3- yl)benzene- sulfonamide | 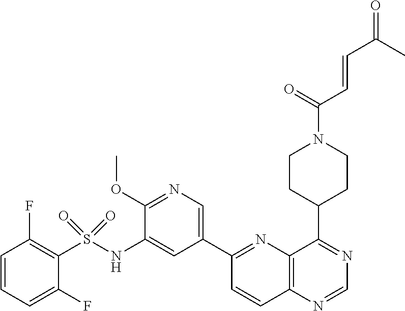 [SMILES: COc1ncc(-c2ccc3ncnc(C4CCN(C(=O)/C=C/C(C)=O)CC4)c3n2)cc1NS(=O)(=O)c1c(F)cccc1F] | ¹H NMR (400 MHz, DMSO-d₆) δ 10.67 (s, 1H), 9.27 (s, 1H), 8.86 (s, 1H), 8.63-8.56 (m, 2H), 8.48 (d, J = 9.2 Hz, 1H), 7.68-7.59 (m, 1H), 7.50 (d, J = 15.6 Hz, 1H), 7.22 (t, J = 9.2 Hz, 2H), 6.70 (d, J = 15.6 Hz, 1H), 4.62 (d, J = 13.2 Hz, 1H), 4.54-4.45 (m, 1H), 4.28 (d, J = 13.2 Hz, 1H), 3.76 (s, 3H), 3.47-3.38 (m, 1H), 3.06- 2.97 (m, 1H), 2.37 (s, 3H), 2.08 (d, J = 13.2 Hz, 2H), 1.97-1.88 (m, 1H), 1.83-1.74 (m, 1H); ESI-MS (m/z): 609.0 [M + H]⁺. |
| Example 234 | (E)-2,4- difluoro-N-(2- methoxy-5- (4-(1-(4- oxopent-2- enoyl)piperidin- 4-yl)pyrido[3,2- d]pyrimidin-6- yl)pyridin-3- yl)benzene- sulfonamide | 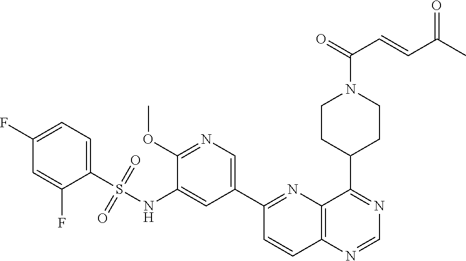 [SMILES: COc1ncc(-c2ccc3ncnc(C4CCN(C(=O)/C=C/C(C)=O)CC4)c3n2)cc1NS(=O)(=O)c1ccc(F)cc1F] | ¹H NMR (400 MHz, DMSO-d₆) δ 10.36 (s, 1H), 9.27 (s, 1H), 8.81 (s, 1H), 8.61-8.51 (m, 2H), 8.47 (d, J = 8.8 Hz, 1H), 7.86-7.74 (m, 1H), 7.55- 7.42 (m, 2H), 7.22-7.12 (m, 1H), 6.70 (d, J = 15.6 Hz, 1H), 4.68-4.59 (m, 1H), 4.54-4.42 (m, 1H), 4.34-4.23 (m, 1H), 3.79 (s, 3H), 3.48-3.37 (m, 1H), 3.08-2.94 (m, 1H), 2.37 (s, 3H), 2.14- 2.04 (m, 2H), 1.99-1.86 (m, 1H), 1.86-1.71 (m, 1H); ESI-MS (m/z): 609.0 [M + H]⁺. |
| Example 235 | (E)-2-chloro-6- fluoro-N-(2- methoxy-5-(4- (1-(4-oxopent- 2-enoyl) piperidin-4-yl) pyrido[3,2-d] pyrimidin-6- yl)pyridin-3- yl)benzene- sulfonamide | 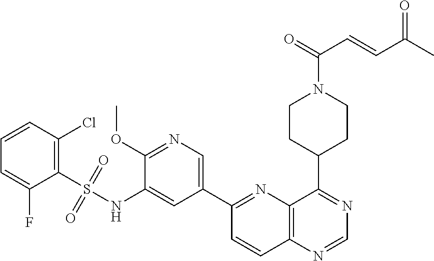 [SMILES: COc1ncc(-c2ccc3ncnc(C4CCN(C(=O)/C=C/C(C)=O)CC4)c3n2)cc1NS(=O)(=O)c1c(F)cccc1Cl] | ¹H NMR (400 MHz, DMSO-d₆) δ 10.57 (s, 1H), 9.28 (s, 1H), 8.97 (d, J = 2.0 Hz, 1H), 8.67 (d, J = 2.4 Hz, 1H), 8.63 (d, J = 9.2 Hz, 1H), 8.50 (d, J = 8.8 Hz, 1H), 7.64 (dd, J = 8.0, 5.2 Hz, 1H), 7.57-7.46 (m, 2H), 7.46-7.27 (m, 1H), 6.71 (d, J = 16.0 Hz, 1H), 4.63 (d, J = 13.2 Hz, 1H), 4.55-4.41 (m, 1H), 4.28 (d, J = 13.6 Hz, 1H), 3.76 (s, 3H), 3.49-3.37 (m, 1H), 3.11- 2.94 (m, 1H), 2.37 (s, 3H), 2.19-2.01 (m, 2H), 1.99-1.87 (m, 1H), 1.85-1.73 (m, 1H); ESI-MS (m/z): 625.0 [M + H]⁺. |

### Example 236

### Preparation of (E)-2,4,6-trifluoro-N-(2-methoxy-5-(4-(1-(4-oxopent-2-enoyl)-1,2,3,6-tetrahydropyridin-4-yl)pyrido[3,2-d]pyrimidin-6-yl)pyridin-3-yl)benzenesulfonamide

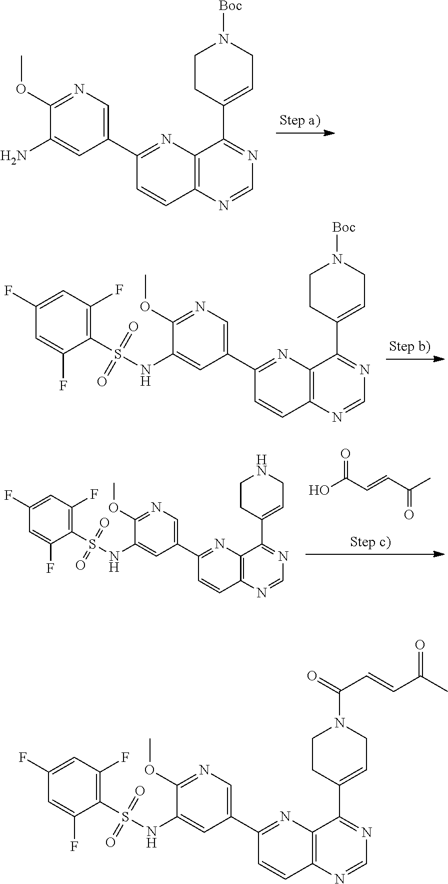

### Step a): Preparation of tert-butyl 4-(6-(6-methoxy-5-((2,4,6-trifluorophenyl)sulfonamido)pyridin-3-yl)pyrido[3,2-d]pyrimidin-4-yl)-3,6-dihydropyridine-1 (2H)-carboxylate

Tert-butyl 4-(6-(5-amino-6-methoxypyridin-3-yl)pyrido[3,2-d]pyrimidin-4-yl)-3,6-dihydropyridine-1 (2H)-carboxylate (90 mg, 0.207 mmol) was added to pyridine (3 mL), then with 2,4,6-trifluorobenzenesulfonyl chloride (143 mg, 0.621 mmol) added. After the addition was completed, the reaction mixture was kept at room temperature and reacted for 2 h. Upon completion of the reaction, the reaction was quenched by adding water (30 mL). The reaction mixture was extracted with ethyl acetate (50 mL×2). The organic phases were combined, washed with saturated brine (50 mL×3), dried over anhydrous sodium sulfate and filtered. The filtrate was concentrated under reduced pressure. The crude product obtained was purified by silica gel column chromatography (Eluent: ethyl acetate/petroleum ether=1/10 to 1/1). Tert-butyl 4-(6-(6-methoxy-5-((2,4,6-trifluorophenyl)sulfonamido)pyridin-3-yl)pyrido[3,2-d]pyrimidin-4-yl)-3,6-dihydropyridine-1 (2H)-carboxylate was obtained, yield: 85.5%; ESI-MS(m/z): 629.2 [M+H]⁺.

### Step b): Preparation of 2,4,6-trifluoro-N-(2-methoxy-5-(4-(1,2,3,6-tetrahydropyridin-4-yl)pyrido[3,2-d]pyrimidin-6-yl)pyridin-3-yl)benzenesulfonamide trifluoroacetate

Tert-butyl 4-(6-(6-methoxy-5-((2,4,6-trifluorophenyl)sulfonamido)pyridin-3-yl)pyrido[3,2-d]pyrimidin-4-yl)-3,6-dihydropyridine-1 (2H)-carboxylate (100 mg, 0.159 mmol) was dissolved in dichloromethane (4 mL). Under the ice bath condition, TFA (1 mL) was slowly added dropwise. After the addition was completed, the reaction mixture was stirred at room temperature for 1 h. Upon completion of the reaction, the reaction mixture was concentrated under reduced pressure. The crude product was slurried with methyl tert-butyl ether (5 mL) to precipitate solid and filtered. 2,4,6-Trifluoro-N-(2-methoxy-5-(4-(1,2,3,6-tetrahydropyridin-4-yl)pyrido[3,2-d]pyrimidin-6-yl)pyridin-3-yl)benzenesulfonamide trifluoroacetate was obtained, yield: 85.2%; ESI-MS(m/z): 529.1 [M+H]⁺.

### Step c): Preparation of (E)-2,4,6-trifluoro-N-(2-methoxy-5-(4-(1-(4-oxopent-2-enoyl)-1,2,3,6-tetrahydropyridin-4-yl)pyrido[3,2-d]pyrimidin-6-yl)pyridin-3-yl)benzenesulfonamide

2,4,6-trifluoro-N-(2-methoxy-5-(4-(1,2,3,6-tetrahydropyridin-4-yl)pyrido[3,2-d]pyrimidin-6-yl)pyridin-3-yl)benzenesulfonamide trifluoroacetate (60 mg, 0.093 mmol) and tetrahydrofuran (2 mL) were added to the reaction flask. The reaction system was cooled to −78° C., with DIPEA (72 mg, 0.558 mmol), (E)-4-oxopent-2-enoic acid (11 mg, 0.093 mmol) and 50% T₃P ethyl acetate solution (118 mg, 0.186 mmol, 50% wt) added successively. After the addition was completed, the reaction mixture was kept at −78° C. and stirred for 1 h. Upon completion of the reaction, the reaction solution was diluted by adding acetonitrile (1 mL), purified by Prep-HPLC (Method 2) and freeze dried. (E)-2,4,6-trifluoro-N-(2-methoxy-5-(4-(1-(4-oxopent-2-enoyl)-1,2,3,6-tetrahydropyridin-4-yl)pyrido[3,2-d]pyrimidin-6-yl)pyridin-3-yl)benzenesulfonamide was obtained, yield: 13.8%; ¹H NMR (400 MHz, DMSO-d_{b}) δ 9.24 (d, J=5.2 Hz, 1H), 8.40-8.36 (m, 2H), 8.28-8.25 (m, 1H), 7.97-7.88 (m, 1H), 7.87-7.79 (m, 1H), 7.56 (dd, J₁=36.4 Hz, J₂=16.0 Hz, 1H), 7.03-6.98 (m, 2H), 6.67 (dd, J₁=16.0 Hz, J₂=12.4 Hz, 1H), 4.71 (d, J=3.6 Hz, 1H), 4.45 (d, J=3.2 Hz, 1H), 3.92-3.83 (m, 2H), 3.82 (d, J=1.6 Hz, 3H), 3.10-3.00 (m, 1H), 2.93-2.82 (m, 1H), 2.39 (d, J=9.6 Hz, 3H); ESI-MS(m/z): 625.0 [M+H]⁺.

Referring to the preparation method of Example 236 and using the corresponding raw materials, the compounds in the following examples were obtained

| Number | Name | Structure | ¹H NMR and MS |
|---|---|---|---|
| Example 237 | (E)-N-(2- methoxy-5-(4-(1- (4-oxopent-2- enoyl)-1,2,3,6- tetrahydropyridin- 4-yl)pyrido[3,2-d] pyrimidin-6- yl)pyridin-3-yl)- 2,4- dimethylthiazole- 5-sulfonamide | 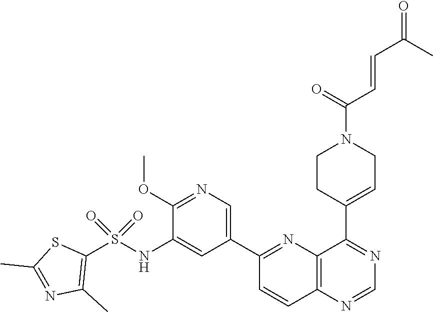 [SMILES: COc1ncc(-c2ccc3ncnc(C4=CCN(C(=O)/C=C/C(C)=O)CC4)c3n2)cc1NS(=O)(=O)c1sc(C)nc1C] | ¹H NMR (400 MHz, DMSO-d₆) δ 10.49 (brs, 1H),, 9.32-9.25 (m, 1H), 8.86-8.77 (m, 1H), 8.65-8.57 (m, 2H), 8.50-8.44 (m, 1H), 7.79-7.74 (m, 1H), 7.60-7.44 (m, 1H), 6.75-6.65 (m, 1H), 4.69-4.62 (m, 1H), 4.53-4.46 (m, 1H), 3.96-3.87 (m, 2H), 3.84 (s, 3H), 3.07 (s, 1H), 2.94 (s, 1H), 2.56-2.52 (m, 3H), 2.42-2.35 (m, 6H); ESI-MS (m/z): 606.0 [M + H]⁺. |
| Example 238 | (E)-2-fluoro-N-(2- methoxy-5-(4-(1- (4-oxopent-2- enoyl)-1,2,3,6- tetrahydropyridin- 4-yl)pyrido[3,2- d]pyrimidin-6- yl)pyridin-3-yl) benzenesulfonamide | 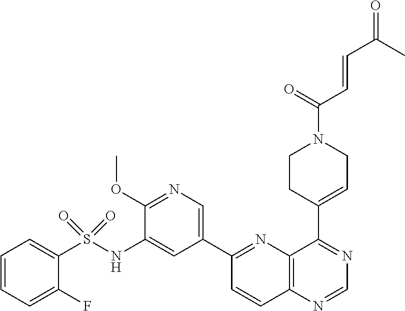 [SMILES: COc1ncc(-c2ccc3ncnc(C4=CCN(C(=O)/C=C/C(C)=O)CC4)c3n2)cc1NS(=O)(=O)c1ccccc1F] | ¹H NMR (400 MHz, DMSO-d₆) δ 9.24 (d, J = 3.2 Hz, 1H), 8.48-8.46 (m, 1H), 8.39-8.36 (m, 2H), 8.22 (s, 1H), 7.81-7.71 (m, 2H), 7.55 (d, J₁ = 39.2, J₂ = 15.6 Hz, 1H), 7.43-7.34 (m, 1H), 7.20-7.08 (m, 2H), 6.67 (dd, J₁ = 9.2 Hz, J₂ = 7.2 Hz, 1H), 4.72 (d, J = 3.6 Hz, 1H), 4.48 (d, J = 3.2 Hz, 1H), 3.92-3.86 (m, 2H), 3.83 (d, J = 4.0 Hz, 3H), 3.12-3.06 (m, 1H), 2.94-2.88 (m, 1H), 2.38 (d, J = 9.6 Hz, 3H); ESI-MS (m/z): 589.0 [M + H]⁺. |
| Example 239 | (E)-2,6-difluoro- N-(2-methoxy-5- (4-(1-(4-oxopent- 2-enoyl)-1,2,3,6- tetrahydropyridin- 4-yl)pyrido[3,2-d] pyrimidin-6- yl)pyridin-3-yl) benzenesulfonamide | 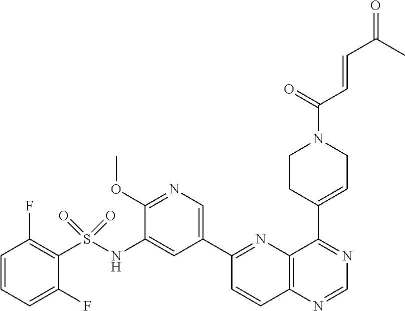 [SMILES: COc1ncc(-c2ccc3ncnc(C4=CCN(C(=O)/C=C/C(C)=O)CC4)c3n2)cc1NS(=O)(=O)c1c(F)cccc1F] | ¹H NMR (400 MHz, DMSO-d₆) δ 10.59 (s, 1H), 9.28 (s, 1H), 8.66 (d, J = 20.8 Hz, 1H), 8.57-8.49 (m, 2H), 8.47-8.42 (m, 1H), 7.83-7.76 (m, 1H), 7.60-7.45 (m, 2H), 7.18-7.08 (m, 2H), 6.70 (dd, J₁ = 15.6 Hz, J₂ = 7.2 Hz, 1H), 4.69-4.63 (m, 1H), 4.49-4.44 (m, 1H), 3.93-3.84 (m, 2H), 3.79-3.76 (m, 3H), 3.04 (s, 1H), 2.90 (s, 1H), 2.38 (d, J = 11.2 Hz, 3H); ESI-MS (m/z): 607.0 [M + H]⁺. |
| Example 240 | (E)-2,4-difluoro- N-(2-methoxy-5- (4-(1-(4-oxopent- 2-enoyl)-1,2,3,6- tetrahydropyridin- 4-yl)pyrido[3,2-d] pyrimidin-6- yl)pyridin-3-yl) benzenesulfonamide | 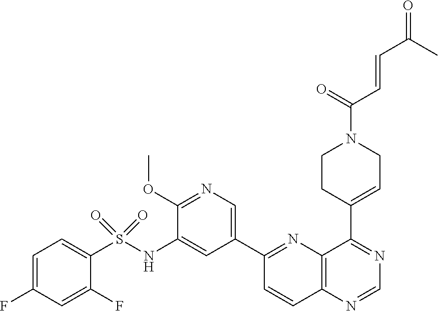 [SMILES: COc1ncc(-c2ccc3ncnc(C4=CCN(C(=O)/C=C/C(C)=O)CC4)c3n2)cc1NS(=O)(=O)c1ccc(F)cc1F] | ¹H NMR (400 MHz, DMSO-d₆) δ 10.43 (s, 1H), 9.26 (d, J = 2.0 Hz, 1H), 8.56-8.35 (m, 4H), 7.85-7.70 (m, 2H), 7.54 (d, J = 16.0 Hz, 1H), 7.29 (s, 1H), 7.04 (d, J = 8.0 Hz, 1H), 6.68 (d, J = 16.0 Hz, 1H), 4.70 (s, 1H), 4.48 (d, J = 3.6 Hz, 1H), 3.95-3.84 (m, 2H), 3.82 (d, J = 3.2 Hz, 3H), 3.07 (s, 1H), 2.91 (s, 1H), 2.39 (d, J = 11.2 Hz, 3H); ESI-MS (m/z): 607.1 [M + H]⁺. |
| Example 241 | (E)-2-chloro-6- fluoro-N-(2- methoxy-5-(4-(1- (4-oxopent-2- enoyl)-1,2,3,6- tetrahydropyridin- 4-yl)pyrido[3,2-d] pyrimidin-6- yl)pyridin-3-yl) benzenesulfonamide | 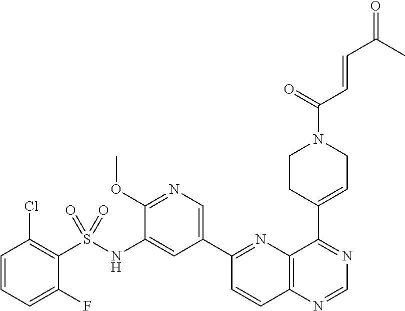 [SMILES: COc1ncc(-c2ccc3ncnc(C4=CCN(C(=O)/C=C/C(C)=O)CC4)c3n2)cc1NS(=O)(=O)c1c(F)cccc1Cl] | ¹H NMR (400 MHz, DMSO-d₆) δ 10.54 (br s, 1H), 9.34-9.26 (m, 1H), 8.90-8.82 (m, 1H), 8.65-8.57 (m, 2H), 8.48 (d, J = 9.2 Hz, 1H), 7.79-7.70 (m, 1H), 7.65-7.42 (m, 3H), 7.41-7.32 (m, 1H), 6.77-6.68 (m, 1H), 4.67-4.44 (m, 2H), 3.96-3.85 (m, 2H), 3.76 (s, 3H), 3.09-2.89 (m, 2H), 2.37 (d, J = 12.4 Hz, 3H); ESI-MS (m/z): 623.0 [M + H]⁺. |

### Example 242

### Preparation of (E)-2,4-difluoro-N-(2-methoxy-5-(7-methyl-4-(4-(4-oxopent-2-enoyl)piperazin-1-yl)quinazolin-6-yl)pyridin-3-yl)benzenesulfonamide

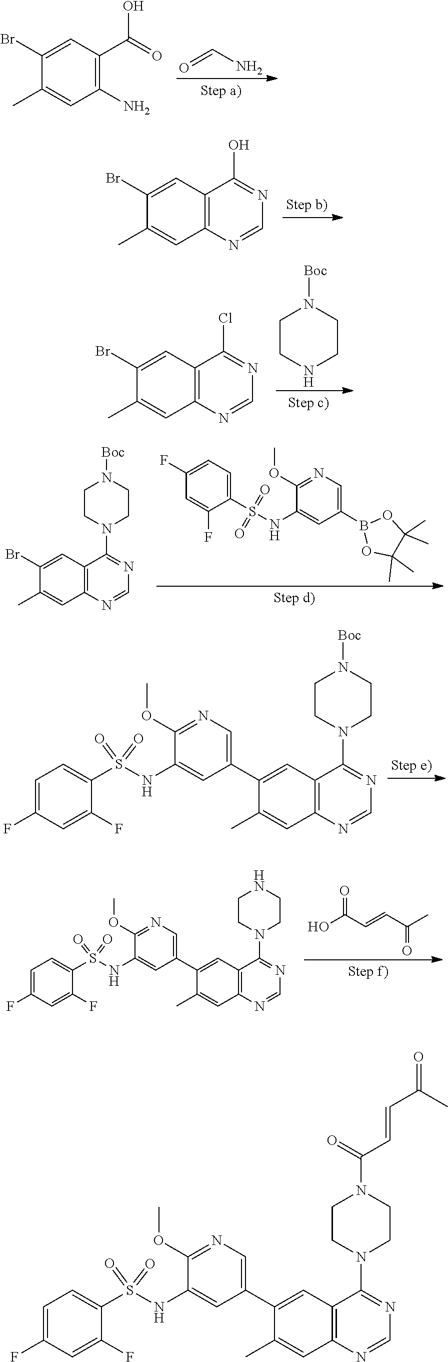

### Step a): Preparation of 6-bromo-7-methylquinazolin-4-ol

2-Amino-5-bromo-4-methylbenzoic acid (I g, 4.347 mmol) and formamide (8 mL) were added to the reaction flask. After the addition was completed, in the presense of protective nitrogen, the reaction mixture was heated to 180° C., stirred and reacted for 8 h. Upon completion of the reaction, the reaction mixture was cooled to room temperature, with reaction quenched by adding water (30 mL). The mixture was stirred for another 0.5 h at room temperature and filtered. The filter cake was dried to obtain 6-bromo-7-methylquinazolin-4-ol, yield: 79.1%; ESI-MS (m/z): 241.1 [M+H]⁺.

### Step b): Preparation of 6-bromo-4-chloro-7-methylquinazoline

6-Bromo-7-methylquinazolin-4-ol (823 mg, 3.443 mmol), thionyl chloride (10 mL) and N,N-dimethylformamide (0.1 mL) were added to the reaction flask. After the addition was completed, the reaction mixture was heated to 110° C., stirred and reacted for 2 h. Upon completion of the reaction, the reaction mixture was cooled to room temperature, concentrated under reduced pressure to remove thionyl chloride. The crude product was dissolved with dichloromethane (20 mL), with reaction quenched by adding iced water (50 mL), adjusted to pH=7-8 with saturated sodium bicarbonate solution and extracted with dichloromethane (50 mL×3). The organic phases were combined, dried over anhydrous sodium sulfate and filtered. The filtrate was concentrated under reduced pressure. 6-Bromo-4-chloro-7-methylquinazoline was obtained. The product can be used directly in the next reaction without further purification; ESI-MS (m/z): 259.0 [M+H]⁺.

### Step c): Preparation of tert-butyl 4-(6-bromo-7-methylquinazolin-4-yl)piperazine-1-carboxylate

6-Bromo-4-chloro-7-methylquinazoline (850 mg, 3.301 mmol), tert-butyl piperazine-1-carboxylate (922 mg, 4.952 mmol), N,N-diisopropylethylamine (1.28 g, 9.903 mmol) and N,N-dimethylformamide (10 mL) were added to the reaction flask. After the addition was completed, the reaction mixture was heated to 50° C., stirred and reacted for 1 h. Upon completion of the reaction. The reaction was quenched by adding water (50 mL) and the reaction mixture was extracted with ethyl acetate (50 mL×3). The organic phases were combined, dried over anhydrous sodium sulfate and filtered. The filtrate was concentrated under reduced pressure. The crude product obtained was purified by silica gel column chromatography (Eluent: petroleum ether/ethyl acetate=10/i to 4/1). Tert-butyl 4-(6-bromo-7-methylquinazolin-4-yl)piperazine-1-carboxylate was obtained, yield: 56.5%; ESI-MS (m/z): 407.1 [M+H]⁺.

### Step d): Preparation of tert-butyl 4-(6-(5-((2,4-difluorophenyl)sulfonamido)-6-methoxypyridin-3-yl)-7-methylquinazolin-4-yl)piperazine-1-carboxylate

Tert-butyl 4-(6-bromo-7-methylquinazolin-4-yl)piperazine-1-carboxylate (150 mg, 0.368 mmol), 2,4-difluoro-N-(2-methoxy-5-(4,4,5,5-tetramethyl-1,3,2-dioxaborolan-2-yl)pyridin-3-yl)benzenesulfonamide (188 mg, 0.442 mmol), Pd(dppf)Cl₂ (54 mg, 0.074 mmol), cesium carbonate (240 mg, 0.736 mmol), 1,4-dioxane (5 mL) and water (0.5 mL) were added to the reaction flask. After the addition was completed, in the presense of protective nitrogen, the reaction mixture was heated to 10° C. and stirred for 3 h. Upon completion of the reaction, the reaction mixture was cooled to room temperature and filtered with diatomaceous earth. The filtrate was concentrated under reduced pressure. The crude product obtained was purified by silica gel column chromatography (Eluent: petroleum ether/ethyl acetate=5/1 to 1/1). Tert-butyl 4-(6-(5-((2,4-difluorophenyl)sulfonamido)-6-methoxypyridin-3-yl)-7-methylquinazolin-4-yl)piperazine-1-carboxylate was obtained, yield: 73.3%; ESI-MS (m/z): 627.3 [M+H]⁺.

### Step e): Preparation of 2,4-difluoro-N-(2-methoxy-5-(7-methyl-4-(piperazin-1-yl)quinazolin-6-yl)pyridin-3-yl)benzenesulfonamide trifluoroacetate

Tert-butyl 4-(6-(5-((2,4-difluorophenyl)sulfonamido)-6-methoxypyridin-3-yl)-7-methylquinazolin-4-yl)piperazine-1-carboxylate (170 mg, 0.271 mmol), trifluoroacetic acid (1.5 mL) and dichloromethane (5 mL) were added to the reaction flask. The reaction mixture was kept at room temperature and stirred for 1 h. Upon completion of the reaction, the reaction mixture was concentrated under reduced pressure. The crude product was slurried by adding methyl tert-butyl ether (5 mL), filtered and dried to obtain 2,4-difluoro-N-(2-methoxy-5-(7-methyl-4-(piperazin-1-yl)quinazolin-6-yl)pyridin-3-yl)benzenesulfonamide trifluoroacetate, yield: 92.5%; ESI-MS (m/z): 527.2 [M+H]⁺.

### Step f): Preparation of (E)-2,4-difluoro-N-(2-methoxy-5-(7-methyl-4-(4-(4-oxopent-2-enoyl)piperazin-1-yl)quinazolin-6-yl)pyridin-3-yl)benzenesulfonamide

2,4-Difluoro-N-(2-methoxy-5-(7-methyl-4-(piperazin-1-yl)quinazolin-6-yl)pyridin-3-yl)benzenesulfonamide trifluoroacetate (110 mg, 0.172 mmol), (E)-4-oxopent-2-enoic acid (22 mg, 0.189 mmol) and tetrahydrofuran (2 mL) were added to the reaction flask. The reaction system was cooled to −78 to, with DIPEA (156 mg, 1.204 mmol) and 50% T₃P ethyl acetate solution (164 mg, 0.258 mmol, 50% wt) added successively. After the addition was completed, the reaction mixture was kept at −78° C. and stirred for 0.5 h. Upon completion of the reaction, the reaction was quenched by adding water (0 mL) into the system. The system was concentrated under reduced pressure at room temperature. The residue was diluted by adding acetonitrile (1 mL) and purified by Prep-HPLC (Method 2). (E)-2,4-difluoro-N-(2-methoxy-5-(7-methyl-4-(4-(4-oxopent-2-enoyl)piperazin-1-yl)quinazolin-6-ylpyridin-3-yl)benzenesulfonamide was obtained, yield: 58.9%; ¹H NMR (400 MHz, DMSO-d₆) δ 10.30 (s, 1H), 8.63 (s, 1H), 8.02 (s, 1H), 7.81-7.73 (m, 3H), 7.62 (s, 1H), 7.52 (t, J=10.0 Hz, 1H), 7.43 (d, J=15.6 Hz, 1H), 7.24-7.17 (m, 1H), 6.71 (d, J=16.0 Hz, 1H), 3.87-3.81 (m, 6H), 3.77-3.73 (m, 2H), 3.71 (s, 3H), 2.36 (s, 3H), 2.32 (s, 3H); ESI-MS(m/z): 623.0 [M+H]⁺.

### Example 243

### Preparation of (E)-2,4-difluoro-N-(2-methoxy-5-(4-(4-(4-oxopent-2-enoyl)piperazin-1-yl)-4-(trifluoromethyl)quinazolin-6-yl)pyridin-3-yl)benzenesulfonamide

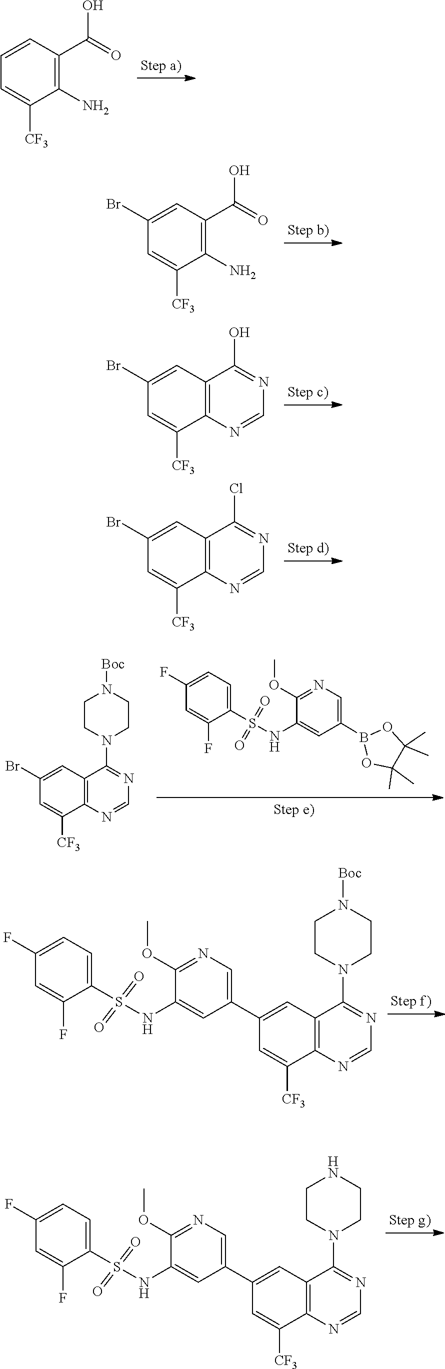

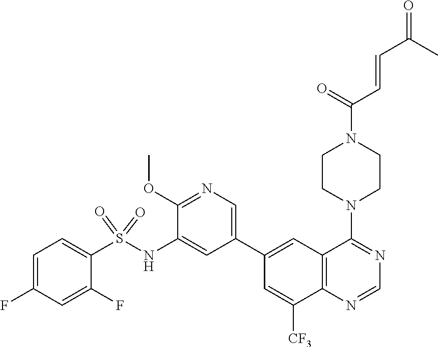 [SMILES: COc1ncc(-c2cc(C(F)(F)F)c3ncnc(N4CCN(C(=O)/C=C/C(C)=O)CC4)c3c2)cc1NS(=O)(=O)c1ccc(F)cc1F]

### Step a): Preparation of 2-amino-5-bromo-3-(trifluoromethyl)benzoic acid

2-Amino-3-(trifluoromethyl)benzoic acid (2.0 g, 9.749 mmol), N,N-dimethylformamide (20 mL) were added to the reaction flask. In the presense of protective nitrogen, N-bromosuccinimide (1.7 g, 9.749 mmol) was slowly added to the system. After the addition was completed, the reaction mixture was kept at room temperature and reacted for 3 h. Upon completion of the reaction, the reaction solution was poured into water and filtered after a large amount of solid precipitation appeared. The filter cake was washed by water (50 mL/2) and dried. 2-Amino-5-bromo-3-(trifluoromethyl)benzoic acid was obtained, yield: 79.4%; ESI-MS(m/z): 282.0 [M−H]⁻.

### Step b): Preparation of 6-bromo-8-(trifluoromethyl)quinazolin-4-ol

2-Amino-5-bromo-3-(trifluoromethyl)benzoic acid (2.2 g, 7.746 mmol), formamide (20 mL) were added to the reaction flask. The reaction mixture was placed in microwave reactor, heated to 180° C. and reacted for 1 h. Upon completion of the reaction, the reaction mixture was cooled to room temperature and filtered after solid precipitation appeared. The filter cake was washed by water (10 mL×3) and dried. 6-Bromo-8-(trifluoromethyl)quinazolin-4-ol was obtained, yield: 83.7%; ESI-MS(m/z): 292.9 [M+H]⁺.

### Step c): Preparation of 6-bromo-4-chloro-8-(trifluoromethyl)quinazoline

6-Bromo-8-(trifluoromethyl)quinazolin-4-ol (1.9 g, 6.483 mmol), thionyl chloride (40 mL). N,N-dimethylformamide (24 mg, 0.324 mmol) were added to the reaction flask. After the addition was completed, the reaction mixture was heated to 100° C. and reacted for 2 h. Upon completion of the reaction, the reaction mixture was concentrated under reduced pressure. 6-Bromo-4-chloro-8-(trifluoromethyl)quinazoline was obtained, yield: 99.0%; ESI-MS(m/z): 311.0 [M+H]⁺.

### Step d): Preparation of tert-butyl 4-(6-bromo-8-(trifluoromethyl)quinazolin-4-yl)piperazine-1-carboxylate

6-Bromo-4-chloro-8-(trifluoromethyl)quinazoline (2 g, 6.421 mmol), N,N-dimethylformamide (20 mL). DIPEA (2.5 g, 19.263 mmol) and tert-butyl piperazine-1-carboxylate (1.3 g, 7.063 mmol) were added to the reaction flask. After the addition was completed, the reaction mixture was heated to 40° C. and reacted for 3 h. Upon completion of the reaction, the reaction was quenched by adding water (50 mL) and the reaction mixture was extracted with ethyl acetate (150 mL 2). The organic phases were combined, washed with saturated brine (100 mL), dried over anhydrous sodium sulfate and filtered. The filtrate was concentrated under reduced pressure. The crude product obtained was purified by silica gel column chromatography (Eluent: petroleum ether/ethyl acetate=10/1 to 2/1). Tert-butyl 4-(6-bromo-8-(trifluoromethyl)quinazolin-4-yl)piperazine-1-carboxylate was obtained, yield: 94.6%; ESI-MS(m/z): 461.2 [M+H]⁺.

### Step e): Preparation of tert-butyl 4-(6-(5-((2,4-difluorophenyl)sulfonamido)-6-methoxypyridin-3-yl)-8-(trifluoromethyl)quinazolin-4-yl)piperazine-1-carboxylate

Tert-butyl 4-(6-bromo-8-(trifluoromethyl)quinazolin-4-yl)piperazine-1-carboxylate (200 mg, 0.434 mmol), 2,4-difluoro-N-(2-methoxy-5-(4,4,5,5-tetramethyl-1,3,2-dioxaborolan-2-yl)pyridin-3-yl)benzenesulfonamide (222 mg, 0.521 mmol), Pd(dppf)Cl₂ (31 mg, 0.043 mmol) cesium carbonate (424 mg, 1.302 mmol) and dioxane/water mixed solvent (10 mL, v/v=4:1) were added to the reaction flask. After the addition was completed, in the presense of protective nitrogen, the reaction mixture was heated to 100° C., stirred and reacted for 3 h. Upon completion of the reaction, the reaction was quenched by adding water (30 mL). The reaction mixture was extracted with ethyl acetate (50 mL×2). The organic phases were combined, washed with saturated brine (30 mL), dried over anhydrous sodium sulfate and filtered. The filtrate was concentrated under reduced pressure. The crude product obtained was purified by silica gel column chromatography (Eluent: methanol/dichloromethane=1/100 to 1/10). Tert-butyl 4-(6-(5-((2,4-difluorophenyl)sulfonamido)-6-methoxypyridin-3-yl)-8-(trifluoromethyl)quinazolin-4-yl)piperazine-1-carboxylate was obtained, yield: 67.8%; ESI-MS(m/z): 681.5 [M+H]⁺.

### Step f): Preparation of 2,4-difluoro-N-(2-methoxy-5-(4-(piperazin-1-yl)-8-(trifluoromethyl)quinazolin-6-yl)pyridin-3-yl)benzenesulfonamide trifluoroacetate

Tert-butyl 4-(6-(5-((2,4-difluorophenyl)sulfonamido)-6-methoxypyridin-3-yl)-8-(trifluoromethyl)quinazolin-4-yl)piperazine-1-carboxylate (200 mg, 0.294 mmol) and dichloromethane (4 mL) were added to the reaction flask. Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure, then with methyl tert-butyl ether (10 mL) added, stirred at room temperature for 0.5 h and filtered after solid precipitation appeared. 2,4-Difluoro-N-(2-methoxy-5-(4-(piperazin-1-yl)-8-(trifluoromethyl)quinazolin-6-yl)pyridin-3-yl)benzenesulfonamide trifluoroacetate was obtained; ESI-MS (m/z): 581.2 [M+H]⁺.

### Step g): Preparation of (E)-2,4-difluoro-N-(2-methoxy-5-(4-(4-(4-oxopent-2-enoyl)piperazin-1-yl)-8-(trifluoromethyl)quinazolin-6-yl)pyridin-3-yl)benzenesulfonamide

2,4-Difluoro-N-(2-methoxy-5-(4-(piperazin-1-yl)-8-(trifluoromethyl)quinazolin-6-yl)pyridin-3-yl)benzenesulfonamide trifluoroacetate (150 mg, 0.162 mmol) and tetrahydrofuran (6 mL) were added to the reaction flask. The reaction system was cooled to −78° C., with DIPEA (105 mg, 0.810 mmol), (E)-4-oxopent-2-enoic acid (20 mg, 0.178 mmol) and 50% T₃P ethyl acetate solution (206 mg, 0.324 mmol, 50% wt) added successively. After the addition was completed, the reaction mixture was kept at −78° C. and stirred for 1 h. Upon completion of the reaction, at −78° C., the reaction was quenched using saturated aqueous ammonium chloride solution (50 mL). The reaction mixture was extracted by adding dichloromethane (100 mL×2). The organic phases were combined, washed with saturated brine (100 mL), dried over anhydrous sodium sulfate and filtered. The filtrate was concentrated under reduced pressure. The crude product obtained was purified by Prep-HPLC (Method 2). (E)-2,4-difluoro-N-(2-methoxy-5-(4-(4-(4-oxopent-2-enoyl)piperazin-1-yl)-8-(trifluoromethyl)quinazolin-6-ylpyridin-3-yl)benzenesulfonamide was obtained, yield: 38.9%; ¹H NMR (400 MHz, DMSO-d₆) δ 10.31 (s, 1H), 8.73 (s, 1H), 8.45-8.26 (m, 3H), 7.97 (s, 1H), 7.85-7.70 (m, 1H), 7.61-7.37 (m, 2H), 7.26-7.08 (m, 1H), 6.74 (d, J=16.0 Hz, 1H), 4.15-3.75 (m, 8H), 3.70 (s, 3H), 2.37 (s, 3H); ESI-MS(m/z): 677.0 [M+H]⁺.

### Example 244

### Preparation of (E)-2,4-difluoro-N-(1-(methyl-d₃)-2-oxo-5-(4-(4-(4-oxopent-2-enoyl)piperazin-1-yl)quinazolin-6-yl)-1,2-dihydropyridin-3-yl)benzenesulfonamide

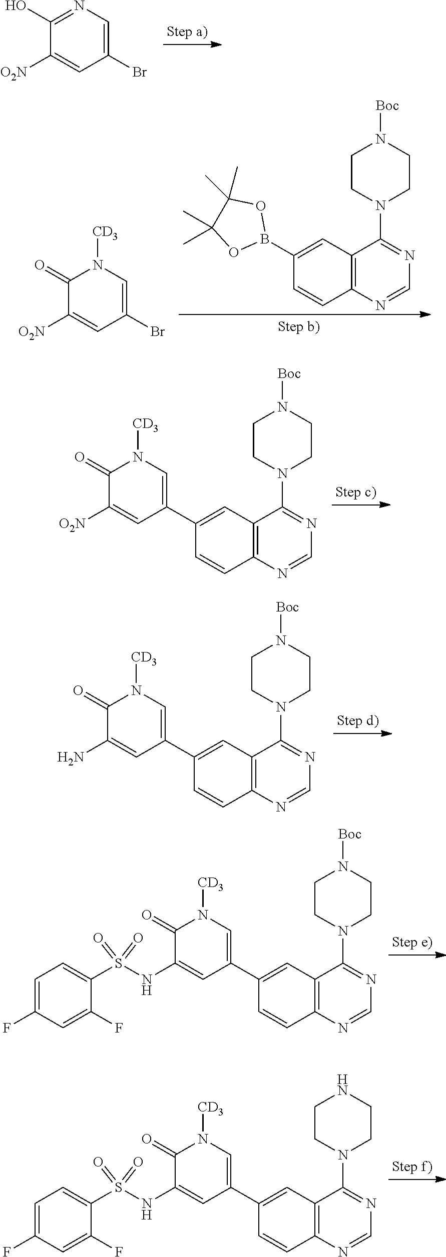

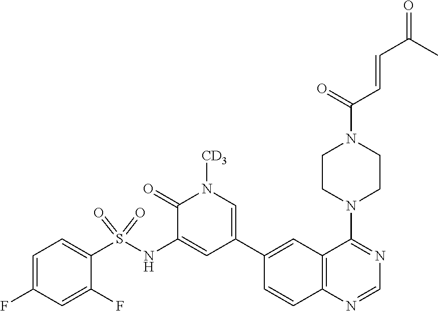 [SMILES: [2H]C([2H])([2H])n1cc(-c2ccc3ncnc(N4CCN(C(=O)/C=C/C(C)=O)CC4)c3c2)cc(NS(=O)(=O)c2ccc(F)cc2F)c1=O]

### Step a): Preparation of 5-bromo-1-(methyl-d₃)-3-nitropyridin-2 (1H)-one

Sodium hydride (73 mg, 1.827 mmol) and N,N-dimethylformamide (6 mL) were added to the reaction flask. The reaction mixture was cooled to 0° C., with 5-bromo-3-nitropyridin-2-ol (200 mg, 0.913 mmol) added dropwise. After the addition was completed, the reaction mixture was kept at 0° C., stirred and reacted for 30 min. with deuterated methyl iodide (265 mg, 1.827 mmol) added dropwise. After the addition was completed, the reaction mixture was stirred at room temperature and reacted for 2 h. Upon completion of the reaction, the reaction was quenched by adding water (3 mL). The reaction mixture was extracted with ethyl acetate (10 mL×3). The organic phases were combined, washed with water (5 mL×3), dried over anhydrous sodium sulfate and filtered. The filtrate was concentrated under reduced pressure. The crude product obtained was purified by silica gel column chromatography (Eluent: petroleum ether/ethyl acetate=10/1 to 3/1), 5-Bromo-1-(methyl-d₃)-3-nitropyridin-2 (1H)-one was obtained, yield: 41.6%; ESI-MS(m/z): 236.1 [M+H]⁺.

### Step b): Preparation of tert-butyl 4-(6-(1-(methyl-d₃)-5-nitro-6-oxo-1,6-dihydropyridin-3-yl)quinazolin-4-yl)piperazine-1-carboxylate

5-Bromo-1-(methyl-d₃)-3-nitropyridin-2 (1H)-one (100 mg, 0.424 mmol), tert-butyl 4-(6-(4,4,5,5-tetramethyl-1,3,2-dioxaborolan-2-yl)quinazolin-4-yl)piperazine-1-carboxylate (187 mg, 0.424 mmol), Pd(dppf)Cl₂ (62 mg, 0.085 mmol), cesium carbonate (276 mg, 0.848 mmol) and dioxane/water mixed solvent (3.3 mL, v/v=10:1) were added to the reaction flask. After the addition was completed, in the presense of protective nitrogen, the reaction mixture was heated to 100° C., stirred and reacted for 3 h. Upon completion of the reaction, the reaction was quenched by adding water (3 mL) and the reaction mixture was extracted with dichloromethane (10 mL×3). The organic phases were combined, dried over anhydrous sodium sulfate and filtered. The filtrate was concentrated under reduced pressure. The crude product obtained was purified by silica gel column chromatography (Eluent: dichloromethane/methanol=1/0 to 10/1). Tert-butyl 4-(6-(1-(methyl-d₃)-5-nitro-6-oxo-1,6-dihydropyridin-3-yl)quinazolin-4-yl)piperazine-1-carboxylate was obtained, yield: 74.1%; ESI-MS(m/z): 470.3 [M+H]⁺.

### Step c): Preparation of tert-butyl 4-(6-(5-amino-1-(methyl-d₃)-6-oxo-1,6-dihydropyridin-3-yl)quinazolin-4-yl)piperazine-1-carboxylate

Tert-butyl 4-(6-(1-(methyl-d₄)-5-nitro-6-oxo-1,6-dihydropyridin-3-yl)quinazolin-4-yl)piperazine-1-carboxylate (100 mg, 0.213 mmol), iron powder (59 mg, 1.065 mmol), ammonium chloride (2 mg, 0.043 mmol) and ethanol/water mixed solvent (4 mL, v/v=3:1) were added to the reaction flask. After the addition was completed, in the presense of protective nitrogen, the reaction mixture was heated to 80° C. and stirred for 3 h. Upon completion of the reaction, the reaction mixture was filtered. The filtrate was concentrated under reduced pressure. The residue was diluted by adding water (3 mL) and extracted with dichloromethane (5 mL×3). The organic phases were combined, dried over anhydrous sodium sulfate and filtered. The filtrate was concentrated under reduced pressure. Tert-butyl 4-(6-(5-amino-1-(methyl-d₃)-6-oxo-1,6-dihydropyridin-3-yl)quinazolin-4-yl)piperazine-1-carboxylate was obtained, yield: 74.5%; ESI-MS(m/z): 440.0 [M+H]⁺.

### Step d): Preparation of tert-butyl 4-(6-(5-((2,4-difluorophenyl)sulfonamido)-1-(methyl-d₃)-6-oxo-1,6-dihydropyridin-3-yl)quinazolin-4-yl)piperazine-1-carboxylate

Tert-butyl 4-(6-(5-amino-1-(methyl-d₃)-6-oxo-1,6-dihydropyridin-3-yl)quinazolin-4-yl)piperazine-1-carboxylate (80 mg, 0.182 mmol) and 2,4-difluorobenzenesulfonyl chloride (39 mg, 0.182 mmol) were dissolved in pyridine (2 mL). The reaction mixture was heated to 70° C., stirred and reacted for 1 h. Upon completion of the reaction, the reaction mixture was concentrated under reduced pressure. The concentrate was diluted by adding water (3 mL) and extracted with dichloromethane (5 mL×3). The organic phases were combined, washed with water (5 mL×3), dried over anhydrous sodium sulfate and filtered. The filtrate was concentrated under reduced pressure. The crude product obtained was purified by silica gel column chromatography (Eluent: dichloromethane/methanol=1/0 to 10/1). Tert-butyl 4-(6-(5-((2,4-difluorophenyl)sulfonamido)-1-(methyl-d₃)-6-oxo-1,6-dihydropyridin-3-yl)quinazolin-4-yl)piperazine-1-carboxylate was obtained, yield: 21.4%; ESI-MS(m/z): 616.2 [M+H]⁺.

### Step e): Preparation of 2,4-difluoro-N-(1-(methyl-d₃)-2-oxo-5-(4-(piperazin-1-yl)quinazolin-6-yl)-1,2-dihydropyridin-3-yl)benzenesulfonamide trifluoroacetate

Tert-butyl 4-(6-(5-((2,4-difluorophenyl)sulfonamido)-1-(methyl-d₃)-6-oxo-1,6-dihydropyridin-3-yl)quinazolin-4-yl)piperazine-1-carboxylate (30 mg, 0.049 mmol) was dissolved in dichloromethane (2 mL). Under the ice bath condition, trifluoroacetic acid (1 mL) was slowly added dropwise. After the addition was completed, the reaction mixture was stirred at room temperature for 1 h. Upon completion of the reaction, the reaction mixture was concentrated under reduced pressure. 2,4-Difluoro-N-(1-(methyl-d₃)-2-oxo-5-(4-(piperazin-1-yl)quinazolin-6-yl)-1,2-dihydropyridin-3-yl)benzenesulfonamide trifluoroacetate was obtained; ESI-MS (m/z): 516.4 [M+H]⁺.

### Step f): Preparation of (E)-2,4-difluoro-N-(1-(methyl-d)-2-oxo-5-(4-(4-(4-oxopent-2-enoyl)piperazin-1-yl)quinazolin-6-yl)-1,2-dihydropyridin-3-yl)benzenesulfonamide

2,4-Difluoro-N-(1-(methyl-d₃)-2-oxo-5-(4-(piperazin-1-yl)quinazolin-6-yl)-1,2-dihydropyridin-3-yl)benzenesulfonamide trifluoroacetate (300 mg, 0.477 mmol) and tetrahydrofuran (4 mL) were added to the reaction flask. The reaction system was cooled to −60° C. with DIPEA (308 mg, 2.383 mmol). (E)-4-oxopent-2-enoic acid (54 mg, 0.477 mmol) and 50% T₃P ethyl acetate solution (304 mg, 0.477 mmol) added successively. After the addition was completed, the reaction mixture was kept at −60° C. and stirred for 1 h. Upon completion of the reaction, the reaction system was diluted by adding acetonitrile (1 mL) and purified by Prep-HPLC (Method 2). (E)-2,4-difluoro-N-(1-(methyl-d₃)-2-oxo-5-(4-(4-(4-oxopent-2-enoyl)piperazin-1-yl)quinazolin-6-yl)-1,2-dihydropyridin-3-yl)benzenesulfonamide was obtained, yield: 23.9%; ¹H NMR (400 MHz, DMSO-d₆) δ 8.64 (s, 1H), 7.97-7.86 (m, 5H), 7.63 (s, 1H), 7.49-7.37 (m, 2H), 7.20-7.15 (m, 1H), 6.74 (d, J=18.8 Hz, 1H), 3.88-3.80 (m, 8H), 2.37 (s, 3H); ESI-MS(m/z): 612.0 [M+H]⁺.

### Example 245

### Preparation of (E)-2,4-difluoro-N-(2-methoxy-5-(4-(4-(4-oxopent-2-enoyl)piperazin-1-yl)pyrido[3,4-d]pyrimidin-6-yl)pyridin-3-yl)benzenesulfonamide

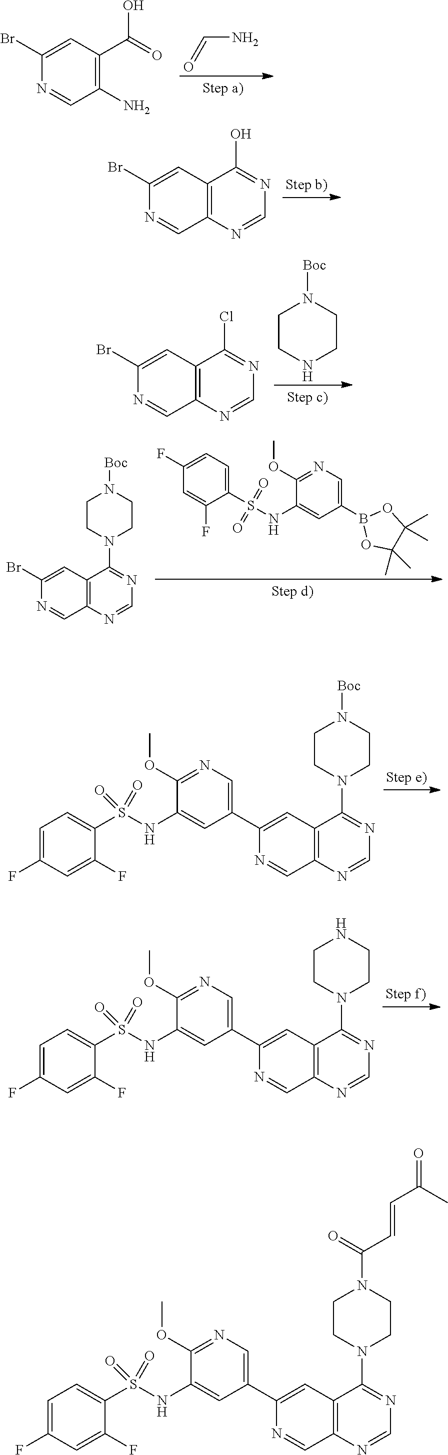

### Step a): Preparation of 6-bromopyrido[3,4-d]pyrimidin-4-ol

5-Amino-2-bromoisonicotinic acid (1 g, 4.608 mmol) and formamide (10 mL) were added to the reaction flask. After the addition was completed, in the presense of protective nitrogen, the reaction mixture was heated to 170° C. and stirred for 6 h. Upon completion of the reaction, the reaction mixture was cooled to room temperature and the resultant precipitation was filtered. The filter cake was washed with water (20 mL) and dried in vacuo. 6-Bromopyrido[3,4-d]pyrimidin-4-ol was obtained, yield: 68.2%; ¹H NMR (400 MHz, DMSO-d_{b}) δ 12.73 (s, 1H), 8.88 (s, 1H), 8.25 (s, 1H), 8.10 (s, 1H).

### Step b): Preparation of 6-bromo-4-chloropyrido[3,4-d]pyrimidine

6-Bromopyrido[3,4-d]pyrimidin-4-ol (350 mg, 1.548 mmol), thionyl chloride (10 mL) and N,N-dimethylformamide (0.1 mL) were added to the reaction flask. After the addition was completed, the reaction mixture was heated to 120° C. and stirred for 12 h. Upon completion of the reaction, the reaction mixture was cooled to room temperature, concentrated under reduced pressure to remove thionyl chloride. 6-Bromo-4-chloropyrido[3,4-d]pyrimidine was obtained; ESI-MS (m/z): 246.0 [M+H]⁺.

### Step c): Preparation of tert-butyl 4-(6-bromopyrido[3,4-d]pyrimidin-4-yl)piperazine-1-carboxylate

6-Bromo-4-chloropyrido[3,4-d]pyrimidine (300 mg, 1.227 mmol), tert-butyl piperazine-1-carboxylate (240 mg, 1.288 mmol), DIPEA (634 mg, 4.908 mmol) and DMF (5 mL) were added to the reaction flask. After the addition was completed, the reaction mixture was heated to 55° C. and stirred for 1 h. Upon completion of the reaction, the reaction was quenched by water (50 mL). The reaction mixture was extracted with ethyl acetate (50 mL×3). The organic phases were combined, dried over anhydrous sodium sulfate and filtered. The filtrate was concentrated under reduced pressure. The crude product obtained was purified by silica gel column chromatography (Eluent: petroleum ether/ethyl acetate=10/1 to 4/1). Tert-butyl 4-(6-bromopyrido[3,4-d]pyrimidin-4-yl)piperazine-1-carboxylate was obtained, yield: 82.7%; ESI-MS (m/z): 394.1 [M+H]⁺.

### Step d): Preparation of tert-butyl 4-(6-(5-((2,4-difluorophenyl)sulfonamido)-6-methoxypyridin-3-yl)pyrido[3,4-d]pyrimidin-4-yl)piperazine-1-carboxylate

Tert-butyl 4-(6-bromopyrido[3,4-d]pyrimidin-4-yl)piperazine-1-carboxylate (200 mg, 0.507 mmol), 2,4-difluoro-N-(2-methoxy-5-(4,4,5,5-tetramethyl-1,3,2-dioxaborolan-2-yl)pyridin-3-yl)benzenesulfonamide (238 mg, 0.558 mmol), Pd(dppf)Cl₂ (37 mg, 0.051 mmol), cesium carbonate (496 mg, 1.521 mmol), 1,4-dioxane (4 mL) and water (1 mL) were added to the reaction flask. After the addition was completed, in the presense of protective nitrogen, the reaction mixture was heated to 100° C. and stirred for 1.5 h. Upon completion of the reaction, the reaction mixture was cooled to room temperature and filtered with diatomaceous earth. The filtrate was concentrated under reduced pressure. The crude product obtained was purified by silica gel column chromatography (Eluent: dichloromethane/methanol=100/1 to 10/1). Tert-butyl 4-(6-(5-((2,4-difluorophenyl)sulfonamido)-6-methoxypyridin-3-yl)pyrido[3,4-d]pyrimidin-4-yl)piperazine-1-carboxylate was obtained, yield: 80.4%; ESI-MS (m/z): 614.3 [M+H]⁺.

### Step c): Preparation of 2,4-difluoro-N-(2-methoxy-5-(4-(piperazin-1-yl)pyrido[3,4-d]pyrimidin-6-yl)pyridin-3-yl)benzenesulfonamide trifluoroacetate

Tert-butyl 4-(6-(5-((2,4-difluorophenyl)sulfonamido)-6-methoxypyridin-3-yl)pyrido[3,4-d]pyrimidin-4-yl)piperazine-1-carboxylate (250 mg, 0.407 mmol) and dichloromethane (3 mL) were added to the reaction flask. At room temperature, trifluoroacetic acid (1 mL) was slowly added dropwise and the mixture was stirred for 1 h. Upon completion of the reaction, the reaction mixture was concentrated under reduced pressure. The crude product was slurried by adding appropriate amount of methyl tert-butyl ether and filtered. The filter cake was dried. 2,4-Difluoro-N-(2-methoxy-5-(4-(piperazin-1-yl)pyrido[3,4-d]pyrimidin-6-yl)pyridin-3-yl)benzenesulfonamide trifluoroacetate was obtained; ESI-MS (m/z): 514.0 [M+H]⁺.

### Step f): Preparation of (E)-2,4-difluoro-N-(2-methoxy-5-(4-(4-(4-oxopent-2-enoyl)piperazin-1-yl)pyrido[3,4-d]pyrimidin-6-yl)pyridin-3-yl)benzenesulfonamide

2,4-Difluoro-N-(2-methoxy-5-(4-(piperazin-1-yl)pyrido[3,4-d]pyrimidin-6-yl)pyridin-3-yl)benzenesulfonamide trifluoroacetate (100 mg, 0.159 mmol) and (E)-4-oxopent-2-enoic acid (20 mg, 0.175 mmol) and tetrahydrofuran (2.5 mL) were added to the reaction flask. The reaction system was cooled to −78° C., with 50% T₃P ethyl acetate solution (152 mg, 0.239 mmol) and DIPEA (164 mg, 1.272 mmol) added successively. After the addition was completed, the reaction mixture was kept at −78° C. and stirred for 0.5 h. Upon completion of the reaction, At −78° C., the reaction was quenched using saturated aqueous ammonium chloride solution (20 mL). The reaction mixture was extracted by adding dichloromethane (30 mL×2). The organic phases were combined, washed with saturated brine (20 mL), dried over anhydrous sodium sulfate and filtered. The filtrate was concentrated under reduced pressure. The crude product obtained was purified by Prep-HPLC (Method 2). (E)-2,4-difluoro-N-(2-methoxy-5-(4-(4-(4-oxopent-2-enoyl)piperazin-1-yl)pyrido[3,4-d]pyrimidin-6-yl)pyridin-3-yl)benzenesulfonamide was obtained, yield: 28.6%. ¹H NMR (400 MHz, DMSO-d₆) δ 10.32 (s, 1H), 9.28 (s, 1H), 8.82-8.79 (m, 1H), 8.76 (s, 1H), 8.39 (d, J=2.4 Hz, 1H), 8.29 (s, 1H), 7.79-7.71 (m, 1H), 7.61-7.54 (m, 1H), 7.44 (d, J=15.6 Hz, 1H), 7.25-7.18 (m, 1H), 6.75 (d, J=15.6 Hz, 1H), 4.13-4.05 (m, 4H), 3.95-3.89 (m, 2H), 3.84-3.78 (m, 2H), 3.69 (s, 3H), 2.37 (s, 3H); ESI-MS(m/z): 610.0 [M+H]⁺.

### Example 246

### Preparation of (E)-S-(2-methoxy-5-(4-(4-(4-oxopent-2-enoyl)piperazin-1-yl)quinazolin-6-yl)pyridin-3-yl)-2,4-difluorobenzenesulfonothioate

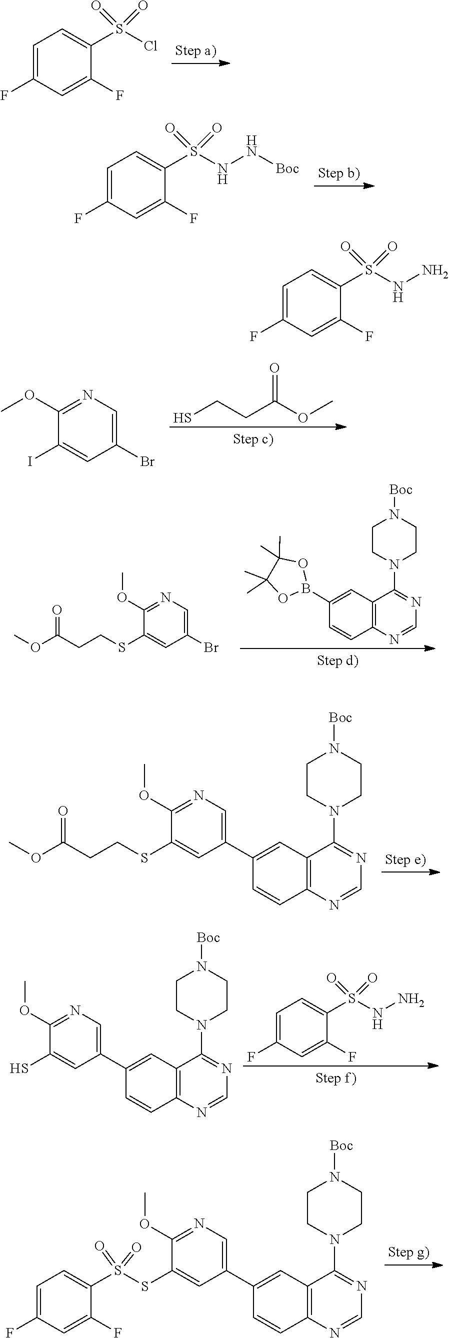

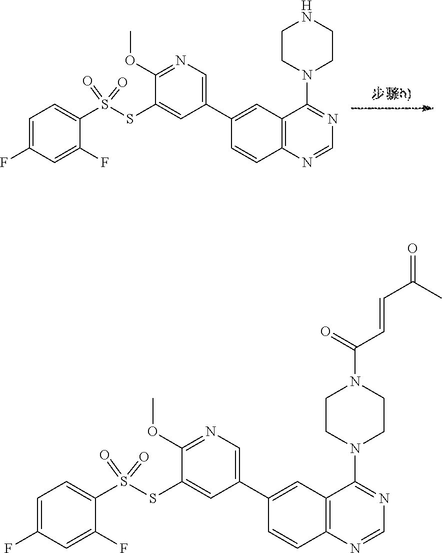 [SMILES: COc1ncc(-c2ccc3ncnc(N4CCNCC4)c3c2)cc1SS(=O)(=O)c1ccc(F)cc1F ; COc1ncc(-c2ccc3ncnc(N4CCN(C(=O)/C=C/C(C)=O)CC4)c3c2)cc1SS(=O)(=O)c1ccc(F)cc1F]

### Step a): Preparation of tert-butyl 2-((2,4-difluorophenyl)sulfonyl)hydrazine-1-carboxylate

Tert-butyl carbazate (5.0 g, 0.038 mol), pyridine (15.0 g, 0.190 mol) and dichloromethane (60 mL) were added to the reaction flask, with 2,4-difluorobenzenesulfonyl chloride (8.5 g, 0.040 mol) slowly added dropwise. After the addition was completed, the reaction mixture was kept at room temperature and reacted for 15 h. Upon completion of the reaction, the reaction was quenched by adding saturated aqueous ammonium chloride solution (100 mL) and the reaction mixture was extracted with dichloromethane (80 mL 2). The organic phases were combined, dried over anhydrous sodium sulfate and filtered. The filtrate was concentrated under reduced pressure. The crude product obtained was purified by silica gel column chromatography (Eluent: petroleum ether/ethyl acetate=1/0 to 10/1). Tert-butyl 2-((2,4-difluorophenyl)sulfonyl)hydrazine-1-carboxylate was obtained, yield: 49.6%; ¹H NMR (400 MHz, Methanol-d₄) δ 7.97-7.82 (m, 1H), 7.25-7.02 (m, 2H), 1.28 (s, 9H).

### Step b): Preparation of 2,4-difluorobenzenesulfonohydrazide

Tert-butyl 2-((2,4-difluorophenyl)sulfonyl)hydrazine-1-carboxylate (4.8 g, 0.016 mol) was dissolved in 4 M HCl ethyl acetate (80 mL). The reaction mixture was stirred at room temperature for 1 h. Upon completion of the reaction, the reaction mixture was concentrated under reduced pressure. The crude product obtained was suspended in tetrahydrofuran (15 mL), with saturated aqueous sodium bicarbonate solution (50 mL) and ethyl acetate (50 mL) added, the aqueous phase was extracted with ethyl acetate (60 mL×2). The organic phases were combined, dried over anhydrous sodium sulfate and filtered. The filtrate was concentrated under reduced pressure. 2,4-Difluorobenzenesulfonohydrazide was obtained, yield: 87.8%; ¹H NMR (400 MHz, DMSO-d₆) δ 8.67 (s, 1H), 7.92-7.78 (m, 1H), 7.59-7.46 (m, 1H), 7.36-7.22 (m, 1H), 4.33 (br s, 2H).

### Step c): Preparation of methyl 3-((5-bromo-2-methoxypyridin-3-yl)thio)propanoate

5-Bromo-3-iodo-2-methoxypyridine (1000 mg, 3.186 mmol), methyl 3-mercaptopropanoate (345 mg, 2.867 mmol), XantPhos (184 mg, 0.319 mmol), Pd(OAc)₂ (72 mg, 0.319 mmol) and DIPEA (1235 mg, 9.558 mmol) were added to the reaction flask, with dioxane (80 mL) added. After the addition was completed, in the presense of protective nitrogen, the reaction mixture was heated to 50° C. and stirred for 2 h. Upon completion of the reaction, the reaction mixture was cooled to room temperature, the reaction was quenched by adding saturated ammonium chloride (50 mL). The reaction mixture was extracted with ethyl acetate (50 mL×3). The organic phases were combined, dried over anhydrous sodium sulfate and filtered. The filtrate was concentrated under reduced pressure. The crude product obtained was purified by silica gel column chromatography (Eluent: petroleum ether/ethyl acetate=1/0 to 10/1). Methyl 3-((5-bromo-2-methoxypyridin-3-yl)thio)propanoate was obtained, yield: 78.3%; ESI-MS(m/z): 305.9 [M+H]⁺.

### Step d): Preparation of tert-butyl 4-(6-(6-methoxy-5-((3-methoxy-3-oxopropyl)thio)pyridin-3-yl)quinazolin-4-yl)piperazine-1-carboxylate

Methyl 3-((5-bromo-2-methoxypyridin-3-yl)thio)propanoate (939 mg, 3.067 mmol), tert-butyl 4-(6-(4,4,5,5-tetramethyl-1,3,2-dioxaborolan-2-yl)quinazolin-4-yl)piperazine-1-carboxylate (1485 mg, 3.374 mmol), Pd(dppf)Cl₂ (224 mg, 0.307 mmol) and sodium bicarbonate (773 mg, 9.201 mmol) were added to the reaction flask, with dioxane (60 mL) and water (0.5 mL) added. After the addition was completed, in the presense of protective nitrogen, the reaction mixture was heated to 60° C. and stirred for 15 h. Upon completion of the reaction, the reaction mixture was cooled to room temperature, the reaction was quenched by adding saturated ammonium chloride (150 mL). The reaction mixture was extracted with ethyl acetate (150 mL×3). The organic phases were combined, dried over anhydrous sodium sulfate and filtered. The filtrate was concentrated under reduced pressure. The crude product obtained was purified by silica gel column chromatography (Eluent: petroleum ether/ethyl acetate=1/0 to 1/2). Tert-butyl 4-(6-(6-methoxy-5-((3-methoxy-3-oxopropyl)thio)pyridin-3-yl)quinazolin-4-yl)piperazine-1-carboxylate was obtained, yield: 29.3%; ESI-MS(m/z): 540.3 [M+H]1.

### Step e): Preparation of tert-butyl 4-(6-(5-mercapto-6-methoxypyridin-3-yl)quinazolin-4-yl)piperazine-1-carboxylate

Tert-butyl 4-(6-(6-methoxy-5-((3-methoxy-3-oxopropyl)thio)pyridin-3-yl)quinazolin-4-yl)piperazine-1-carboxylate (485 mg, 0.899 mmol) was dissolved in tetrahydrofuran (20 mL), with potassium tert-butoxide (345 mg, 3.595 mmol) added. After the addition was completed, the reaction mixture was stirred at room temperature for 0.5 h. Upon completion of the reaction, the reaction was quenched by adding saturated ammonium chloride (20 mL), and the reaction mixture was extracted with dichloromethane (20 mL×3). The organic phases were combined, dried over anhydrous sodium sulfate and filtered. The filtrate was concentrated under reduced pressure.

Tert-butyl 4-(6-(5-mercapto-6-methoxypyridin-3-yl)quinazolin-4-yl)piperazine-1-carboxylate was obtained. The product can be used directly in the next reaction without purification; ESI-MS(m/z): 454.0 [M+H]⁺.

### Step f): Preparation of tert-butyl 4-(6-(5-(((2,4-difluorophenyl)sulfonyl)thio)-6-methoxypyridin-3-yl)quinazolin-4-yl)piperazine-1-carboxylate

Tert-butyl 4-(6-(5-mercapto-6-methoxypyridin-3-yl)quinazolin-4-yl)piperazine-1-carboxylate (408 mg, crude), 2,4-difluorobenzenesulfonohydrazide (282 mg, 1.356 mmol), sodium iodide (68 mg, 0.452 mmol) were added to the reaction flask, with acetonitrile (15 mL) added. Tert-butyl peroxide (204 mg, 2.260 mmol) was slowly added dropwise to the reaction system. After the addition was completed, in the presense of protective nitrogen, the reaction mixture was stirred at room temperature for 15 h. Upon completion of the reaction, the reaction mixture was directly concentrate under reduced pressure. The crude product obtained was purified by silica gel column chromatography (Eluent: dichloromethane/methanol=1/0 to 33/1). Tert-butyl 4-(6-(5-(((2,4-difluorophenyl)sulfonyl)thio)-6-methoxypyridin-3-yl)quinazolin-4-yl)piperazine-1-carboxylate was obtained, two-step yield: 45.0%; ESI-MS(m/z): 630.5 [M+H]r.

### Step g): Preparation of S-(2-methoxy-5-(4-(piperazin-1-yl)quinazolin-6-yl)pyridin-3-yl) 2,4-difluorobenzenesulfonothioate trifluoroacetate

Tert-butyl 4-(6-(5-(((2,4-difluorophenyl)sulfonyl)thio)-6-methoxypyridin-3-yl)quinazolin-4-yl)piperazine-1-carboxylate (150 mg, 0.238 mmol) was dissolved in dichloromethane (9 mL). Under the ice bath condition, TFA (3 mL) was slowly added dropwise. After the addition was completed, the reaction mixture was stirred at room temperature for 1 h. Upon completion of the reaction, the reaction mixture was concentrated under reduced pressure. The crude product obtained was dispersed in 10 mL methyl tert-butyl ether, stirred at room temperature for 0.5 h. The solid was collected and dried. S-(2-methoxy-5-(4-(piperazin-1-yl)quinazolin-6-yl)pyridin-3-yl) 2,4-difluorobenzenesulfonothioate trifluoroacetate was obtained, yield: 98.9%; ESI-MS(m/z): 530.1 [M+H]⁺.

### Step h): Preparation of (E)-S-(2-methoxy-5-(4-(4-(4-oxopent-2-enoyl)piperazin-1-yl)quinazolin-6-yl)pyridin-3-yl) 2,4-difluorobenzenesulfonothioate

S-(2-methoxy-5-(4-(piperazin-1-yl)quinazolin-6-yl)pyridin-3-yl) 2,4-difluorobenzenesulfonothioate trifluoroacetate (150 mg, 0.233 mmol), (E)-4-oxopent-2-enoic acid (30 mg, 0.257 mmol), DIPEA (150 mg, 1.165 mmol) were placed to the reaction flask, with tetrahydrofuran (6 mL) added. At −78° C., T₃P (193 mg, 0.303 mmol, 50% wt ethyl acetate solution) was slowly added to the system. After the addition was completed, the reaction mixture was kept at −78° C. and stirred for 1 h. Upon completion of the reaction the reaction was quenched by adding saturated ammonium chloride (40 mL) and the reaction mixture was extracted with dichloromethane (40 mL×2). The organic phases were combined, dried over anhydrous sodium sulfate and filtered. The filtrate was concentrated under reduced pressure. The crude product obtained was purified by Prep-HPLC (Method 2). (E)-S-(2-methoxy-5-(4-(4-(4-oxopent-2-enoyl)piperazin-1-yl)quinazolin-6-yl)pyridin-3-yl) 2,4-difluorobenzenesulfonothioate was obtained, yield: 25.2%; ¹H NMR (400 MHz, DMSO-d₆) δ 8.85 (d, J=2.4 Hz, 1H), 8.77 (s, 1H), 8.48 (d, J=2.4 Hz, 1H), 8.28 (br s, 1H), 8.22 (d, J=8.8 Hz, 1H), 7.94 (d, J=8.8 Hz, 1H), 7.81-7.70 (m, 1H), 7.64-7.54 (m, 1H), 7.44 (d, J=16.0 Hz, 1H), 7.28-7.19 (m, 1H), 6.76 (d, J=15.6 Hz, 1H), 4.16-4.02 (m, 4H), 3.96-3.87 (m, 2H), 3.86-3.77 (m, 2H), 3.60 (s, 3H), 2.37 (s, 31H); ESI-MS(m/z): 626.0 [M+H]⁺.

### Example 247

### Preparation of (E)-2,4-difluoro-N-(2-methoxy-5-(4-(1-(4-oxopent-2-enoyl)pyrrolidin-3-yl)quinazolin-6-yl)pyridin-3-yl)benzenesulfonamide

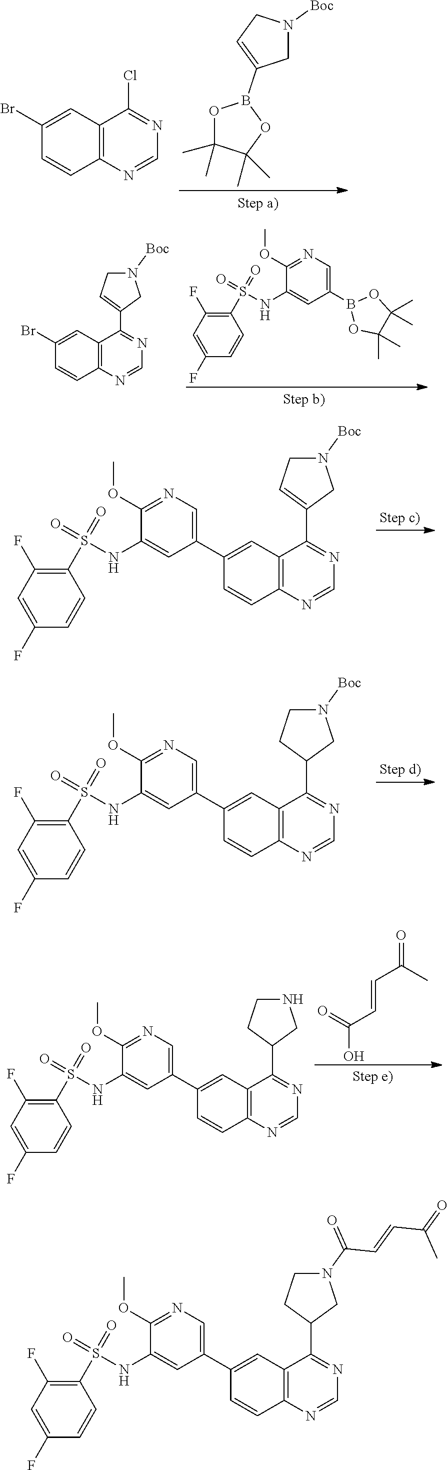

### Step a): Preparation of tert-butyl 3-(6-bromoquinazolin-4-yl)-2,5-dihydro-1H-pyrrole-1-carboxylate

6-Bromo-4-chloroquinazoline (500 mg, 2.053 mmol), tert-butyl 3-(4,4,5,5-tetramethyl-1,3,2-dioxaborolan-2-yl)-2,5-dihydro-1H-pyrrole-1-carboxylate (606 mg, 2.053 mmol), Pd(dppf)Cl₂ (150 mg, 0.205 mmol) and cesium carbonate (2.01 g, 6.159 mmol) were added to 1,4-dioxane/water mixed solvent (20 mL, v/v=10/1). After the addition was completed, in the presense of protective nitrogen, the reaction mixture was kept at room temperature and reacted overnight. Upon completion of the reaction, the reaction was quenched by adding water (50 mL). The reaction mixture was extracted with ethyl acetate (80 mL×2). The organic phases were combined, washed with saturated brine (50 mL), dried over anhydrous sodium sulfate and filtered. The filtrate was concentrated under reduced pressure. The crude product obtained was purified by silica gel column chromatography (Eluent: methanol/dichloromethane=1/100 to 1/10). Tert-butyl 3-(6-bromoquinazolin-4-yl)-2,5-dihydro-1H-pyrrole-1-carboxylate was obtained, yield: 67.5%; ESI-MS(m/z): 376.1 [M+H]⁺.

### Step b): Preparation of tert-butyl 3-(6-(5-((2,4-difluorophenyl)sulfonamido)-6-methoxypyridin-3-yl)quinazolin-4-yl)-2,5-dihydro-1H-pyrrole-1-carboxylate

Tert-butyl 3-(6-bromoquinazolin-4-yl)-2,5-dihydro-1H-pyrrole-1-carboxylate (300 mg, 0.797 mmol), 2,4-difluoro-N-(2-methoxy-5-(4,4,5,5-tetramethyl-1,3,2-dioxaborolan-2-yl)pyridin-3-yl)benzenesulfonamide (340 mg, 0.797 mmol), Pd(dppf)Cl₂ (59 mg, 0.080 mmol) and cesium carbonate (779 mg, 2.391 mmol) were added to 1,4-dioxane/water mixed solvent (10 mL, v/v=10/1). After the addition was completed, in the presense of protective nitrogen, the reaction mixture was heated to 100° C. and reacted for 3 h. Upon completion of the reaction, the reaction was quenched by adding water (50 mL). The reaction mixture was extracted with ethyl acetate (100 mL×2). The organic phases were combined, washed with saturated brine (50 mL), dried over anhydrous sodium sulfate and filtered. The filtrate was concentrated under reduced pressure. The crude product obtained was purified by silica gel column chromatography (Eluent: dichloromethane/methanol=100/1 to 10/1). Tert-butyl 3-(6-(5-((2,4-difluorophenyl)sulfonamido)-6-methoxypyridin-3-yl)quinazolin-4-yl)-2,5-dihydro-1H-pyrrole-1-carboxylate was obtained, yield: 74.3%; ESI-MS(m/z): 596.2 [M+H]⁺.

### Step c): Preparation of tert-butyl 3-(6-(5-((2,4-difluorophenyl)sulfonamido)-6-methoxypyridin-3-yl)quinazolin-4-yl)pyrrolidine-1-carboxylate

Tert-butyl 3-(6-(5-((2,4-difluorophenyl)sulfonamido)-6-methoxypyridin-3-yl)quinazolin-4-yl)-2,5-dihydro-1H-pyrrole-1-carboxylate (200 mg, 0.336 mmol), 10% Pd/C (100 mg), glacial acetic acid (0.2 mL) were added to methanol (20 mL), with hydrogen purged three times, under hydrogen atmosphere, the reaction mixture was kept at 30° C. and react for 3 h. Upon completion of the reaction, the reaction solution was filtered. The filtrate was concentrated under reduced pressure. The residue was diluted by adding water (30 mL) and extracted with ethyl acetate (50 mL×2). The organic phases were combined, washed with saturated brine (30 mL), dried over anhydrous sodium sulfate and filtered. The filtrate was concentrated under reduced pressure. Tert-butyl 3-(6-(5-((2,4-difluorophenyl)sulfonamido)-6-methoxypyridin-3-yl)quinazolin-4-yl)pyrrolidine-1-carboxylate was obtained, yield: 86.3%; ESI-MS(m/z): 598.2 [M+H]⁺.

### Step d): Preparation of 2,4-difluoro-N-(2-methoxy-5-(4-(pyrrolidin-3-yl)quinazolin-6-yl)pyridin-3-yl)benzenesulfonamide trifluoroacetate

Tert-butyl 3-(6-(5-((2,4-difluorophenyl)sulfonamido)-6-methoxypyridin-3-yl)quinazolin-4-yl)pyrrolidine-1-carboxylate (150 mg, 0.251 mmol) was dissolved in dichloromethane (4 mL). Under the ice bath condition, TFA (1 mL) was slowly added dropwise. After the addition was completed, the reaction mixture was stirred at room temperature for 1 h. Upon completion of the reaction, the reaction mixture was concentrated under reduced pressure. The crude product was slurried with methyl tert-butyl ether (5 mL) to precipitate solid and filtered. 2,4-Difluoro-N-(2-methoxy-5-(4-(pyrrolidin-3-yl)quinazolin-6-yl)pyridin-3-yl)benzenesulfonamide trifluoroacetate was obtained; ESI-MS(m/z): 498.2 [M+H]⁺.

### Step e): Preparation of (E)-2,4-difluoro-N-(2-methoxy-5-(4-(1-(4-oxopent-2-enoyl)pyrrolidin-3-yl)quinazolin-6-yl)pyridin-3-yl)benzenesulfonamide

2,4-Difluoro-N-(2-methoxy-5-(4-(pyrrolidin-3-yl)quinazolin-6-yl)pyridin-3-yl)benzenesulfonamide trifluoroacetate (80 mg, 0.131 mmol) was dissolved in tetrahydrofuran (2 mL). The reaction system was cooled to −78° C., with DIPEA (102 mg, 0.786 mmol), (E)-4-oxopent-2-enoic acid (15 mg, 0.131 mmol) and 50% T₃P ethyl acetate solution (167 mg, 0.262 mmol, 50% wt) added successively. After the addition was completed, the reaction mixture was kept at −78° C. and reacted for 1 h. Upon completion of the reaction, the reaction solution was diluted by adding acetonitrile (1 mL), purified by Prep-HPLC (Method 2) and freeze dried. (E)-2,4-difluoro-N-(2-methoxy-5-(4-(1-(4-oxopent-2-enoyl)pyrrolidin-3-yl)quinazolin-6-yl)pyridin-3-yl)benzenesulfonamide was obtained, yield: 18.0%; ¹H NMR (400 MHz, DMSO-d₆) δ 10.34 (br s, 1H), 9.24 (d, J=5.2 Hz, 1H), 8.67-8.56 (m, 2H), 8.33-8.29 (m, 1H), 8.14-8.10 (m, 1H), 7.79-7.74 (m, 1H), 7.60-7.54 (m, 1H), 7.24 (dd, J₁=15.6, J₂=4.4 Hz, 1H), 7.21-7.15 (m, 1H), 6.77 (dd, J₁=15.6. J₂=13.2 Hz, 2H), 4.85-4.74 (m, 1H), 4.33-4.18 (m, 1H), 4.09-3.84 (m, 3H), 3.73-3.68 (m, 1H), 3.67 (d, J=2.0 Hz, 3H), 3.64-3.57 (m, 1H), 2.36 (d, J=18.0 Hz, 3H); ESI-MS(m/z): 594.0 [M+H]⁺.

### Example 248

### Preparation of (Z)-2,4-difluoro-N-(2-methoxy-5-(4-(4-(5,5,5-trifluoro-4-oxopent-2-enoyl)piperazin-1-yl)quinazolin-6-yl)pyridin-3-yl)benzenesulfonamide

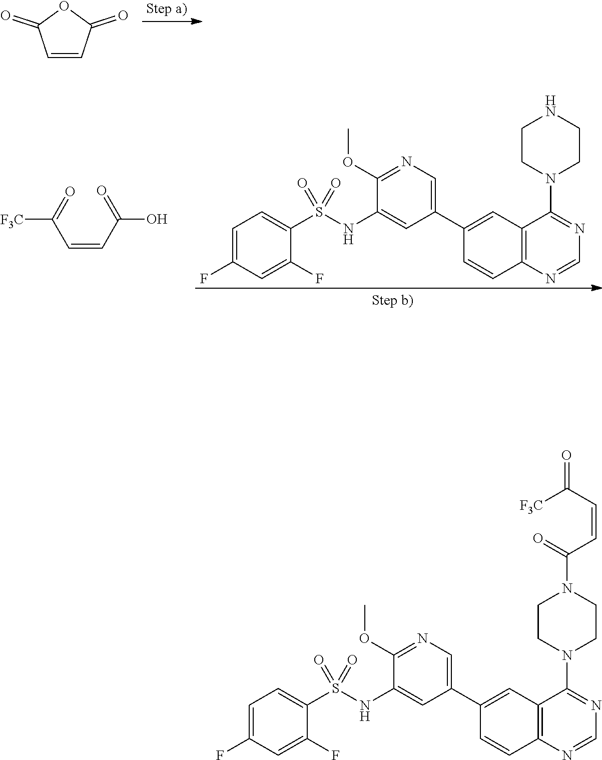 [SMILES: O=C1C=CC(=O)O1 ; O=C(O)/C=C\C(=O)C(F)(F)F ; COc1ncc(-c2ccc3ncnc(N4CCNCC4)c3c2)cc1NS(=O)(=O)c1ccc(F)cc1F ; COc1ncc(-c2ccc3ncnc(N4CCN(C(=O)/C=C\C(=O)C(F)(F)F)CC4)c3c2)cc1NS(=O)(=O)c1ccc(F)cc1F]

### Step a): Preparation of (Z)-5,5,5-trifluoro-4-oxopent-2-enoic acid

In the presense of protective nitrogen, furan-2,5-dione (5.0 g, 0.051 mol) and cesium fluoride (9.3 g, 0.061 mol) was suspended in acetonitrile (60 mL) at 0° C. (Trifluoromethyl)trimethylsilane (8.7 g, 0.061 mol) was slowly added dropwise to the system. After the addition was completed, in the presense of protective nitrogen, the reaction mixture was kept at 0° C. and stirred for 1 h. Upon completion of the reaction, at 0° C., the reaction was quenched by adding 2 M aqueous sodium hydroxide solution (60 mL) and the reaction solution was extracted with methyl tert-butyl ether (100 mL/2). The organic was discarded. The aqueous phase was adjusted to pH=1 with 40% sulfuric acid and extracted with dichloromethane (50 mL×10). The organic phases were combined, washed with saturated brine (100 mL), dried over anhydrous sodium sulfate and filtered. The filtrate was concentrated under reduced pressure. The crude product obtained was purified by silica gel column chromatography (Eluent: petroleum ether/ethyl acetate=1/0 to 2/1). (Z)-5,5,5-trifluoro-4-oxopent-2-enoic acid was obtained, yield: 14.0%; ¹H NMR (400 MHz, DMSO-d₆) δ 9.45 (s, 1H), 7.82 (d, J=5.6 Hz, 1H), 6.75 (d, J=5.6 Hz, 1H); ESI-MS(m/z): 167.0 [M−H]⁻.

### Step b): Preparation of (Z)-2,4-difluoro-N-(2-methoxy-5-(4-(4-(5,5,5-trifluoro-4-oxopent-2-enoyl)piperazin-1-yl)quinazolin-6-yl)pyridin-3-yl)benzenesulfonamide

2,4-Difluoro-N-(2-methoxy-5-(4-(piperazin-1-yl)quinazolin-6-yl)pyridin-3-yl)benzenesulfonamide trifluoroacetate (100 mg, 0.160 mmol), (Z)-5,5,5-trifluoro-4-oxopent-2-enoic acid (27 mg, 0.160 mmol), N,N-diisopropylethylamine (83 mg, 0.640 mmol) were added to DMF (5 mL). After the addition was completed, under the ice bath condition, HATU (61 mg, 0.160 mmol) was added. The reaction mixture was kept under the ice bath condition and reacted for 1 h. Upon completion of the reaction, the reaction was quenched by adding water (30 mL), and the reaction mixture was extracted with dichloromethane (50 mL×2). The organic phases were combined, washed with saturated brine (30 mL), dried over anhydrous sodium sulfate and filtered. The filtrate was concentrated under reduced pressure. The crude product obtained was purified by Prep-HPLC (Method 2). (Z)-2,4-difluoro-N-(2-methoxy-5-(4-(4-(5,5,5-trifluoro-4-oxopent-2-enoyl)piperazin-1-yl)quinazolin-6-yl)pyridin-3-yl)benzenesulfonamide was obtained, yield: 23.5%; ¹H NMR (400 MHz, DMSO-d₆) δ 10.29 (s, 1H), 8.66 (s, 1H), 8.45-8.32 (m, 1H), 8.15-8.00 (m, 3H), 8.00-7.93 (m, 1H), 7.91 (d, J=9.2 Hz, 1H), 7.82-7.72 (m, 1H), 7.58-7.45 (m, 1H), 7.24-7.15 (m, 1H), 6.79 (d, J=5.6 Hz, 1H), 3.97-3.75 (m, 4H), 3.69 (s, 3H), 3.15-2.93 (m, 4H); ESI-MS(m/z): 663.0 [M+H]⁺.

### Bioactivity Testing

The positive reference used in the present disclosure of PI3Kα is the compound with following structure (Alpelisib, NVP-BYL-719):

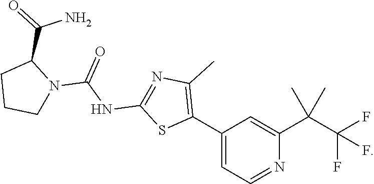 [SMILES: Cc1nc(NC(=O)N2CCC[C@H]2C(N)=O)sc1-c1ccnc(C(C)(C)C(F)(F)F)c1]

The positive reference used in the present disclosure of PI3Kδ is the compound with following structure (Linperlisib, YY-20394):

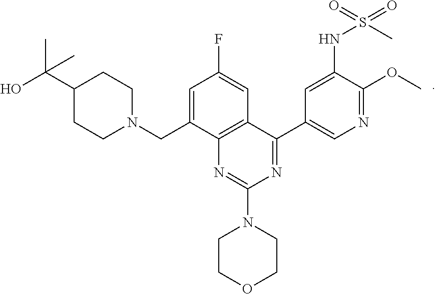 [SMILES: COc1ncc(-c2nc(N3CCOCC3)nc3c(CN4CCC(C(C)(C)O)CC4)cc(F)cc23)cc1NS(C)(=O)=O]

or the compound with following structure (Parsaclisib, IBI-376):

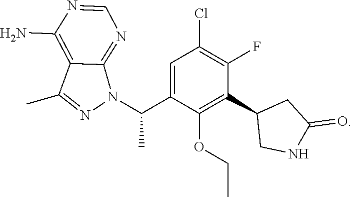 [SMILES: CCOc1c([C@H](C)n2nc(C)c3c(N)ncnc32)cc(Cl)c(F)c1[C@@H]1CNC(=O)C1]

The positive reference used in the present disclosure of G12C is the compound with following structure (AMG-510):

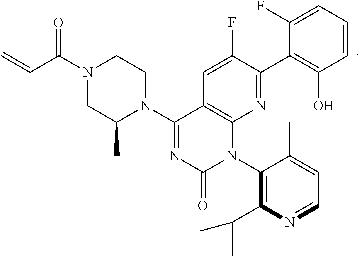

### Test Example 1: Inhibition of PI3Kα Efficacy assay

The ADP-Glo™ kinase assay was used to assess the inhibition of PI3Kα with our compounds. The utilized proteins and assay reagents were obtained from the ADP-Glo™ Kinase Assay Kit (Promega). Initially, a 2 mM stock solution of the test compound (dissolved in DMSO) was serially diluted in five-fold concentrations using DMSO to obtain eight working solutions 1 (200′). Subsequently, each of the eight working solutions 1 was further diluted in a 20-fold gradient by adding 5 μL into 95 μL of ddH₂O, resulting in eight working solutions 2 (10×). In a 384-well white flat-bottom plate, 2 μL of 2.5×PI3Kα kinase solution and 1 μL of working solution 2 (10×) were added to each well and mixed, followed by a 15-minute incubation at room temperature (RT). Subsequently, 2 μL of 2.5×PI and ATP solution mixture were added to each well, mixed, and incubated at RT for 60 minutes. Then, 5 μL of ADP-Glo (containing 10 mM MgCl₂) reagent was added to each well, mixed, and incubated at RT for 40 minutes. Finally, 10 μL of the Kinase Detection Reagent was added to each well, mixed, and incubated at RT for 40 minutes. Luminescence values were recorded using a multi-mode microplate reader with luminescence channel settings, and the inhibitory rate was calculated using the following formula:

$$\% Inhibition rate = [ ( Negative - test compound ) / ( Negative - Blank ) ] * 100$$

Data were analyzed using GraphPad Prism with log(inhibitor) vs. response—Variable Slope(four parameters) fitting calculations for IC₅ values. (Note: The negative control refers to the group without inhibitor, and the blank control refers to the group without enzyme.) The data were presented in Tables 1 to 4.

### Test Example 2: Inhibition of PI3Kδ Efficacy assay

The ADP-Glo™ kinase assay was used to assess the inhibition of PI3Kδ with our compounds. The utilized proteins and assay reagents were obtained from the ADP-Glo™ Kinase Assay Kit (Promega). Initially, a 2 mM stock solution of the test compound (dissolved in DMSO) was serially diluted in five-fold concentrations using DMSO to obtain eight working solutions 1 (200×) Subsequently, each of the eight working solutions 1 was further diluted in a 20-fold gradient by adding 5 μL into 95 μL of ddH₂O, resulting in eight working solutions 2 (10×). In a 384-well white flat-bottom plate, 2 μL of 2.5×PI3Kδ kinase solution and 1 μL of working solution 2 (10×) were added to each well and mixed, followed by a 15-minute incubation at room temperature (RT). Subsequently, 2 μL of 2.5×PI and ATP solution mixture were added to each well, mixed, and incubated at RT for 60 minutes. Then, 5 μL of ADP-Glo (containing 10 mM MgCl₂) reagent was added to each well, mixed, and incubated at RT for 40 minutes. Finally, 10 μL of the Kinase Detection Reagent was added to each well, mixed, and incubated at RT for 40 minutes. Luminescence values were recorded using a multi-mode microplate reader with luminescence channel settings, and the inhibitory rate was calculated using the following formula:

$$\% Inhibition rate = [ ( Negative - test compound ) / ( Negative - Blank ) ] * 100$$

Data were analyzed using GraphPad Prism with log(inhibitor) vs. response—Variable Slope(four parameters) fitting calculations for IC₅₀ values. (Note: The negative control refers to the group without inhibitor, and the blank control refers to the group without enzyme. The data were resented in Tables 1 to 4.

TABLE 1
Inhibitory activity of compounds against
PI3Kα and PI3Kδ

| | PI3Kα | PI3Kδ |
|---|---|---|
| | inhibitory | inhibitory |
| Example | activity | activity |
| Number | (IC₅₀, nM) | (IC₅₀, nM) |
| Example 1 | 0.26 | 2.35 |
| Example 2 | 7.3 | 41.8 |
| Example 4 | 9.5 | 12.0 |
| Example 6 | 1.5 | 10.9 |
| Example 7 | 1.4 | 12.0 |
| Example 8 | 0.34 | 17.9 |
| Example 9 | 0.56 | 9.05 |
| Example 10 | 0.44 | 16.7 |
| Example 11 | 0.64 | 9.2 |
| Example 12 | 9.3 | 77.4 |
| Example 13 | 11.3 | 85.3 |
| Example 14 | 9.5 | 62.1 |
| Example 15 | 10.2 | 130.4 |
| Example 16 | 0.26 | 28.5 |
| Example 17 | 2.3 | 31.0 |
| Example 18 | 1.2 | 12.5 |
| Example 19 | 4.7 | 57.8 |
| Example 20 | 7.4 | 211.8 |
| Example 21 | 5.8 | 58.2 |
| Example 22 | 11.6 | 82.8 |
| Example 24 | 2.9 | 6.3 |
| Example 25 | 26.9 | 126.6 |
| Example 26 | 44.6 | 265.1 |
| Example 27 | 1.8 | 27.3 |
| Example 28 | 1.8 | 49.7 |
| Example 29 | 41.3 | 160.0 |
| Example 30 | 430.4 | 1835 |
| Example 31 | 148.6 | 335.6 |
| Example 32 | 2264 | >5000 |
| Example 33 | 12.6 | 68.7 |
| Example 34 | 1.4 | 20.8 |
| Example 35 | 5.8 | 116.9 |
| Example 36 | 21.5 | 139.6 |
| Example 37 | 1.3 | 20.1 |
| Example 38 | 1.5 | 14.7 |
| Example 41 | 7.6 | 10.1 |
| Alpelisib | 18.7 | N.T. |
| Linperlisib | N.T. | 18.0 |

Note:
N.T. represents not tested.

TABLE 2
Inhibitory activity of compounds
against PI3Kα and PI3Kδ

| | PI3Kα | PI3Kδ |
|---|---|---|
| | inhibitory | inhibitory |
| Example | activity | activity |
| Number | (IC₅₀, nM) | (IC₅₀, nM) |
| Example 42 | 1.5 | 16.5 |
| Example 43 | 2.5 | 12.9 |
| Example 44 | 4.3 | 17.5 |
| Example 45 | 2.8 | 38.6 |
| Example 46 | 1.7 | 36.3 |
| Example 47 | 2.2 | 25.2 |
| Example 48 | 0.63 | 13.2 |
| Example 49 | 2.6 | 14.9 |
| Example 50 | 0.61 | 11.4 |
| Example 51 | 5.6 | 19.2 |
| Example 52 | 9.1 | N.T. |
| Example 53 | 4.4 | N.T. |
| Example 54 | 2.5 | N.T. |
| Example 55 | 1.2 | 43.5 |
| Example 56 | 4.4 | 37.2 |
| Example 57 | 1.9 | 33.4 |
| Example 58 | 3.6 | 8.3 |
| Example 59 | 3.3 | 13.1 |
| Example 60 | 3.9 | 13.5 |
| Example 61 | 5.2 | N.T. |
| Example 62 | 11.8 | 12.5 |
| Example 63 | 6.2 | N.T. |
| Example 64 | 16.7 | 14.6 |
| Example 65 | 3.9 | 53.0 |
| Example 66 | 13.8 | 14.8 |
| Example 67 | 9.8 | 13.4 |
| Example 68 | 1.4 | 27.7 |
| Example 69 | 2.1 | 15.5 |
| Example 70 | 8.8 | 35.0 |
| Example 71 | 2.1 | 31.5 |
| Example 72 | 5.9 | 24.3 |
| Example 73 | 2.5 | 16.5 |
| Example 74 | 1.8 | 23.2 |
| Example 75 | 0.78 | 31.2 |
| Example 76 | 7.5 | 25.3 |
| Example 77 | 3.9 | N.T. |
| Example 79 | 909.8 | 903 |
| Example 82 | 89.9 | 75.6 |
| Example 83 | 6.2 | 17.6 |
| Example 84 | 0.83 | 7.6 |
| Example 85 | 2.8 | N.T. |
| Example 86 | 0.31 | N.T. |
| Example 87 | 2.4 | N.T. |
| Example 88 | 2.6 | 11.8 |
| Example 89 | 2.2 | 8.6 |
| Example 90 | 4.0 | 23.7 |
| Example 91 | 2.6 | 61.5 |
| Example 92 | 6.4 | 51.1 |
| Example 93 | 6.0 | 72.9 |
| Example 94 | 11.0 | 56.7 |
| Example 95 | 414.1 | N.T. |
| Example 96 | 1.0 | 171.0 |
| Example 97 | 1.4 | 14.1 |
| Example 98 | 9.8 | N.T. |
| Example 99 | 6.0 | 7.9 |
| Example 100 | 0.54 | N.T. |
| Example 101 | 9.9 | 33.6 |
| Example 102 | 1.2 | 32.8 |
| Example 103 | 0.95 | 32.2 |
| Example 104 | 3.8 | 32.2 |
| Example 105 | 8.6 | 38.5 |
| Example 106 | 117.4 | 116.9 |
| Example 107 | 125.6 | 794.0 |
| Example 108 | 161.3 | 267.6 |
| Example 109 | 3.5 | 48.9 |
| Example 110 | 3.5 | N.T. |
| Example 111 | 8.8 | 23.2 |
| Example 112 | 5.0 | 23.9 |
| Example 113 | 2.9 | 11.5 |
| Example 114 | 1.4 | 28.5 |
| Example 115 | 1.9 | N.T. |
| Example 116 | 1.8 | 27.0 |
| Example 117 | 1.5 | N.T. |
| Example 118 | 1.9 | 19.7 |
| Example 119 | 2.8 | 11.8 |
| Example 120 | 3.0 | 11.5 |
| Example 121 | 111.3 | N.T. |
| Example 122 | 8.1 | 4.7 |
| Example 123 | 9.1 | 31.4 |
| Example 124 | 4.4 | 61.4 |
| Example 125 | 5.0 | 17.0 |
| Example 126 | 130.7 | 52.7 |
| Example 127 | 1.9 | 31.7 |
| Example 128 | 21.7 | N.T. |
| Example 129 | 106.9 | 141.5 |
| Example 130 | 2.6 | 15.2 |
| Alpelisib | 11.0 | N.T. |
| Parsaclisib | N.T. | 8.0 |

TABLE 3
Inhibitory activity of compounds
against PI3Kα and PI3Kδ

| | PI3Kα | PI3Kδ |
|---|---|---|
| | inhibitory | inhibitory |
| Example | activity | activity |
| Number | (IC₅₀, nM) | (IC₅₀, nM) |
| Example 131 | 1.6 | 13.7 |
| Example 132 | 3.6 | 29.2 |
| Example 133 | 5.4 | 20.4 |
| Alpelisib | 10.0 | N.T. |
| Parsaclisib | N.T. | 6.0 |

TABLE 4
Inhibitory activity of compounds
against PI3Kα and PI3Kδ

| | P13Kα | PI3Kδ |
|---|---|---|
| | inhibitory | inhibitory |
| Example | activity | activity |
| Number | (IC₅₀, nM) | (IC₅₀, nM) |
| Example 140 | 0.43 | N.T. |
| Example 141 | 0.30 | N.T. |
| Example 142 | 0.57 | N.T. |
| Example 144 | 3.2 | 13.4 |
| Example 145 | 0.44 | 23.8 |
| Example 146 | 2.1 | N.T. |
| Example 147 | 1.6 | N.T. |
| Example 148 | 9.2 | N.T. |
| Example 149 | 3.3 | N.T. |
| Example 150 | 0.11 | 10.7 |
| Example 151 | 5.9 | N.T. |
| Example 152 | 0.28 | 15.5 |
| Example 153 | 0.73 | 12.7 |
| Example 154 | 0.54 | 13.8 |
| Example 155 | 0.30 | 53.1 |
| Example 156 | 0.50 | N.T. |
| Example 157 | 1.3 | N.T |
| Example 158 | 3.0 | 34.6 |
| Example 159 | 11.8 | 91.7 |
| Example 160 | 2.0 | 15.2 |
| Example 161 | 0.82 | 9.9 |
| Example 162 | 2.4 | N.T. |
| Example 163 | 0.25 | N.T. |
| Example 164 | 2.8 | N.T. |
| Example 165 | 1.2 | 12.0 |
| Example 166 | 16.1 | 22.3 |
| Example 167 | 2.2 | 11.8 |
| Example 168 | 6.2 | 17.2 |
| Example 169 | 3.8 | 18.9 |
| Example 170 | 7.0 | 17.0 |
| Example 171 | 12.9 | 40.1 |
| Example 172 | 4.1 | 11.3 |
| Example 173 | 2.2 | 16.5 |
| Example 174 | 3.5 | 17.1 |
| Example 175 | 6.0 | 16.2 |
| Example 176 | 2.6 | 13.9 |
| Example 177 | 5.0 | 19.1 |
| Example 178 | 20.5 | 9.2 |
| Example 179 | 0.37 | N.T. |
| Example 180 | 1.8 | N.T. |
| Example 181 | 0.73 | 20.4 |
| Example 182 | 0.65 | N.T. |
| Example 183 | 5.3 | N.T. |
| Example 184 | 0.81 | 16.2 |
| Example 185 | 1.6 | N.T. |
| Example 186 | 2.4 | N.T. |
| Example 187 | 4.6 | N.T. |
| Example 188 | 3.3 | N.T. |
| Example 189 | 3.1 | N.T. |
| Example 190 | 9.5 | N.T. |
| Example 191 | 6.4 | 16.7 |
| Example 192 | 9.3 | N.T. |
| Example 193 | 2.8 | N.T. |
| Example 194 | 2.8 | N.T. |
| Example 195 | 2.4 | 13.5 |
| Example 196 | 1.7 | N.T. |
| Example 197 | 4.1 | 8.6 |
| Example 198 | 1.1 | 13.3 |
| Example 199 | 1.5 | 12.0 |
| Example 200 | 2.3 | 9.7 |
| Example 201 | 3.8 | 11.8 |
| Example 202 | 1.9 | 27.9 |
| Example 203 | 2.4 | 10.4 |
| Example 204 | 3.3 | 17.0 |
| Example 205 | 1.3 | 9.0 |
| Example 206 | 3.9 | 15.0 |
| Example 207 | 3.2 | N.T. |
| Example 208 | 30.6 | N.T. |
| Example 209 | 0.57 | N.T. |
| Example 210 | 0.54 | 5.9 |
| Example 211 | 1.7 | N.T. |
| Example 212 | 3.8 | N.T. |
| Example 213 | 9.8 | N.T. |
| Example 214 | 2.1 | N.T. |
| Example 215 | 2.4 | N.T. |
| Example 216 | 1.0 | N.T. |
| Example 217 | 0.41 | 10.6 |
| Example 218 | 2.6 | 10.8 |
| Example 219 | 0.91 | 11.6 |
| Example 220 | 1.1 | 11.4 |
| Example 221 | 2.2 | N.T. |
| Example 222 | 0.49 | 11.7 |
| Example 223 | 1.4 | N.T. |
| Example 224 | 1.1 | 23.8 |
| Example 225 | 0.50 | 8.9 |
| Example 226 | 1.4 | N.T. |
| Example 227 | 2.4 | N.T. |
| Example 228 | 2.8 | 15.9 |
| Example 229 | 3.0 | N.T. |
| Example 230 | 2.6 | 4.7 |
| Example 231 | 3.6 | 16.1 |
| Example 232 | 2.0 | 14.6 |
| Example 233 | 4.0 | 31.3 |
| Example 234 | 4.2 | 20.0 |
| Example 235 | 0.3 | 2.38 |
| Example 236 | 1.7 | N.T. |
| Example 237 | 5.2 | N.T. |
| Example 238 | 3.1 | N.T. |
| Example 239 | 6.4 | N.T. |
| Example 240 | 5.0 | N.T. |
| Example 241 | 0.27 | 2.96 |
| Example 242 | 282.7 | N.T. |
| Example 243 | 838.1 | N.T. |
| Example 244 | 270.0 | N.T. |
| Example 245 | 14.9 | N.T. |
| Example 246 | 2.24 | 13.77. |
| Example 247 | 0.53 | 3.33 |
| Example 248 | 2.3 | N.T. |
| Alpelisib | 8.0 | N.T. |
| Parsaclisib | N.T. | 4.0 |

Alpelisib, Linperlisib/Parsaclisib were known as PI3Kα inhibitors and PI3Kδ inhibitors, respectively. From the above data, it can be observed that the compounds of the present disclosure demonstrate significant inhibitory effects on PI3Kα and PI3Kδ. Some of the compounds exhibit superior activity compared to the positive control compounds and can be utilized as PI3K inhibitors. They hold promising prospects for wide application in the field of diseases mediated by PI3K proteins, such as cancer.

### Test Example 3: Enzymatic Activity of Compounds Against KRAS^{G12C} In Vitro (KRAS^{G12C}-SOS1 Binding Testing Method)

The HTRF KRAS^{G12C}/SOS1 Binding Assay was used to measure the interaction between KRAS^{G12C} and SOS1 proteins, and evaluate the enzymatic activity of compounds against KRAS^{G12C}. The utilized proteins and assay reagents were obtained from the KRAS^{G12C}-SOS1 binding assay kits (Cisbio). Initially, a 2 mM stock solution of the test compound (dissolved in DMSO) was serially diluted in five-fold concentrations using DMSO to obtain eight working solutions 1 (200×). Subsequently, each of the eight working solutions 1 was further diluted in a 20-fold gradient by adding 5 μL into 95 μL of Diluent buffer, mixed completely with vortex mixer, resulting in eight working solutions 2 (10×). In a 384-well white flat-bottom plate, 4 μL of Tag2-KRAS^{G12C} solution, 2 μL of working solution 2 (10×) and 4 μL of Tag1-SOS1 solution were sequentially added to each well and mixed, followed by a 15-minute incubation at room temperature (RT). Subsequently, 5 μL of Anti-tag 1-Tb³⁺ stock solution and 5 μL of Anti-tag2-XL665 were added to each well, mixed, and incubated at 4° C. for 3 hours. Remove the plate and read on an HTRF® compatible reader, with the excitation light wavelength set at 337 nm and readings recorded at 620 nm and 665 nm. The data results were presented as the ratio of the 665 nm signal value to the 620 nm signal value for each well, calculated as follows: Ratio=104/(665 nm signal value)/(620 nm signal value). The inhibition rate was calculated using the following formula:

$$\% Inhibition rate = [ ( {Ratio}_{negative} - {Ratio}_{compound} ) / ( {Ratio}_{negative} - {Ratio}_{Blank} ) ] \times 100$$

Data were analyzed using GraphPad Prism with log (inhibitor) vs. response-Variable Slope (four parameters) fitting calculations for IC₅₀ values. (Note: The negative control refers to the group without inhibitor, and the blank control refers to the group without enzyme.) The data were presented in Table 5.

TABLE 5
Inhibitory activity of compounds on
KRAS-G12C/SOS1 binding

| | Kras G12C |
|---|---|
| | binding assay |
| Compound | (IC₅₀, nM) |
| Example 6 | 323 |
| Example 8 | 221.5 |
| Example 13 | 96.71 |
| Example 15 | 557.8 |
| Example 17 | 313 |
| Example 20 | 239.4 |
| Example 22 | 149.0 |
| Example 34 | 521.1 |
| Example 38 | 807.1 |
| Example 42 | 109.0 |
| Example 46 | 166.5 |
| Example 49 | 225.3 |
| Example 51 | 941.6 |
| Example 56 | 470.9 |
| Example 58 | 278.1 |
| Example 84 | 267.7 |
| Example 88 | 218.9 |
| Example 89 | 403.6 |
| Example 91 | 173.2 |
| Example 92 | 369.0 |
| Example 93 | 344.4 |
| Example 94 | 188.8 |
| Example 97 | 101.2 |
| Example 99 | 188.4 |
| Example 101 | 197.9 |
| Example 102 | 140.6 |
| Example 103 | 202.7 |
| Example 104 | 168.6 |
| Example 109 | 498.9 |
| Example 111 | 364.5 |
| Example 116 | 1573 |
| Example 122 | 145.2 |
| Example 124 | 729.6 |
| Example 131 | 200.0 |
| Example 132 | 317.8 |
| Example 133 | 250.6 |
| Example 144 | 389.9 |
| Example 145 | 200.2 |
| Example 150 | 236.1 |
| Example 152 | 174.8 |
| Example 153 | 210.0 |
| Example 154 | 183.4 |
| Example 155 | 461.8 |
| Example 158 | 190.6 |
| Example 160 | 168.9 |
| Example 161 | 283.9 |
| Example 165 | 362.2 |
| Example 168 | 546.3 |
| Example 169 | 249.2 |
| Example 171 | 441.3 |
| Example 172 | 622.0 |
| Example 173 | 198.9 |
| Example 174 | 422.7 |
| Example 175 | 287.5 |
| Example 176 | 113.3 |
| Example 177 | 246.2 |
| Example 178 | 213.3 |
| Example 179 | 147.2 |
| Example 184 | 365.0 |
| Example 191 | 143.0 |
| Example 195 | 158.4 |
| Example 197 | 271.0 |
| Example 198 | 133.4 |
| Example 199 | 176.2 |
| Example 200 | 133.3 |
| Example 201 | 140.3 |
| Example 202 | 142.2 |
| Example 203 | 419.2 |
| Example 204 | 306.1 |
| Example 205 | 369.1 |
| Example 206 | 188.3 |
| Example 210 | 130.7 |
| Example 217 | 207.4 |
| Example 218 | 291.8 |
| Example 219 | 151.5 |
| Example 220 | 274.0 |
| Example 223 | 210.3 |
| Example 224 | 187.5 |
| Example 225 | 109.5 |
| Example 228 | 293.8 |
| Example 230 | 493.1 |
| Example 231 | 462.5 |
| Example 232 | 269.2 |
| Example 233 | 203.6 |
| Example 234 | 292.0 |
| Example 235 | 112.6 |
| Example 241 | 454.5 |
| Example 246 | 159.0 |
| Example 247 | 277.1 |
| / | / |

From the above data, it can be seen that the compounds of the present disclosure also have obvious inhibitory effects on KRAS G12C and have broad application prospects in disease-related fields such as cancer mediated by KRAS G12C protein.

### Test Example 4: Determination of the inhibitory effect on proliferation activity of KRAS G12C mutant cell line NCI-H358 cells

The CellTiter-Glo®, assay was used to detect the number of viable cells, in order to evaluate the anti-proliferation efficacy of compounds. NCI-H358 cells in logarithmic growth phase were collected and seeded in a clear-bottom 96-well plate at a density of 4×10³ cells per well in 80 μL of medium, and then incubated overnight at 37° C. with 5% CO₂. The compounds were serially diluted 5-fold in DMSO to obtain 8 concentrations, and then further diluted with RPMI-640 (10% FBS) cell culture medium to obtain the working solutions (5′), 20 μL of each working solution was added to the cell culture supernatant in each well, and the plate was further incubated for 3 days at 37° C. with 5% CO₂. The plate was then equilibrated with CellTiter-Glo® reagent at room temperature (25° C.) for 10-30 minutes. After aspirating 50 μL of the medium, 50 μL of CellTiter-Glo® reagent was added. The cells were thoroughly mixed with the CellTiter-Glo® reagent using a microplate shaker for 2 minutes, and then incubated at room temperature for 10 minutes. The 96-well plate was placed in a multi-mode microplate reader to record the luminescence values (RLU).

The inhibition rate was calculated through the following equation:

$$\% Inhibition rate = ⌊ \frac{{\overline{Ratio}}_{Negative reference} - {\overline{Ratio}}_{Tested compound}}{{\overline{Ratio}}_{Negative reference}} ⌋ * 100$$

Data were analyzed using GraphPad Prism for IC₅₀ values (Note: The negative control refers to the group without inhibitor). The data were presented in Table 6.

### Test Example 5: Determination of the Anti-Proliferation Activity Against the MIA Paca-2 Cells Cell Line (KRAS G12C Mutant)

The number of viable cells was determined using the CellTiter-Glo® assay to evaluate the anti-proliferation efficacy of compounds. MIA PaCa-2 cells in logarithmic growth phase were collected and seeded in a clear-bottom 384-well plate at a density of 600 cells per well in 15 μL of medium, and then incubated overnight at 37° C. with 5% CO₂. The compounds were serially diluted 4-fold in DMSO to obtain 9 concentrations, and then further diluted with DMEM (10% FBS+2.5% horse serum+1% penicillin/streptomycin) cell culture medium to obtain the working solutions (2×), 15 μL of each working solution was added to the cell culture supernatant in each well, and the cells were further incubated for 3 days at 37° C. with 5% CO₂. The plate was then equilibrated with CellTiter-Glo® reagent at room temperature (25° C.) for 30 minutes. After adding 25 μL of CellTiter-Glo®, reagent, the cells were thoroughly mixed with the CellTiter-Glo® reagent using a microplate shaker for 2 minutes, and then incubated at room temperature for at least 30 minutes. The 384-well plate was placed in a multi-mode microplate reader to record the luminescence values (RLU).

The inhibition rate was calculated through the following equation:

$$\% Inhibition rate = ⌊ \frac{{\overline{Ratio}}_{Negative reference} - {\overline{Ratio}}_{Tested compound}}{{\overline{Ratio}}_{Negative reference}} ⌋ * 100$$

Data were analyzed using GraphPad Prism for IC₅₀ values (Note: The negative control refers to the group without inhibitor). The data were presented in Table 7.

TABLE 6
Inhibitory activity of compounds on
H358 cells (G12C mutation)

| Example | NCI-H358 |
|---|---|
| Number | (IC₅₀, nM) |
| Example 1 | 21.0 |
| Example 2 | 485.0 |
| Example 4 | 494.0 |
| Example 5 | 5387 |
| Example 6 | 18.0 |
| Example 7 | 44.0 |
| Example 8 | 91.2 |
| Example 9 | 349.6 |
| Example 10 | 24.6 |
| Example 11 | 101.7 |
| Example 12 | 50.6 |
| Example 13 | 73.3 |
| Example 14 | 232.9 |
| Example 15 | 19.9 |
| Example 16 | 35.2 |
| Example 17 | 34.6 |
| Example 18 | 60.1 |
| Example 19 | 22.0 |
| Example 20 | 26.2 |
| Example 21 | 27.0 |
| Example 22 | 23.7 |
| Example 24 | 123.2 |
| Example 25 | 288.4 |
| Example 26 | 141.7 |
| Example 33 | 333.3 |
| Example 35 | 899.9 |
| Example 36 | 278.1 |
| Example 41 | 4293 |
| Example 42 | 79.0 |
| Example 43 | 27.9 |
| Example 44 | 41.6 |
| Example 45 | 163.7 |
| Example 46 | 29.9 |
| Example 47 | 34.3 |
| Example 48 | 52.6 |
| Example 49 | 32.0 |
| Example 50 | 31.1 |
| Example 51 | 48.7 |
| Example 53 | 520.0 |
| Example 54 | 10.4 |
| Example 55 | 39.5 |
| Example 56 | 69.7 |
| Example 57 | 29.9 |
| Example 58 | 18.1 |
| Example 59 | 30.6 |
| Example 60 | 48.2 |
| Example 61 | 291.8 |
| Example 62 | 15.1 |
| Example 63 | 19.6 |
| Example 64 | 12.6 |
| Example 65 | 25.1 |
| Example 66 | 13.5 |
| Example 67 | 24.2 |
| Example 68 | 861.4 |
| Example 69 | 60.7 |
| Example 70 | 58.4 |
| Example 71 | 75.0 |
| Example 72 | 14.4 |
| Example 73 | 34.3 |
| Example 74 | 26.5 |
| Example 75 | 285.0 |
| Example 76 | 3416 |
| Example 77 | 220.7 |
| Example 80 | 784.4 |
| Example 81 | 1089 |
| Example 83 | 458.8 |
| Example 84 | 13.0 |
| Example 85 | 213.1 |
| Example 86 | 271.4 |
| Example 87 | 130.7 |
| Example 88 | 80.9 |
| Example 89 | 81.4 |
| Example 90 | 54.5 |
| Example 91 | 17.8 |
| Example 92 | 16.7 |
| Example 93 | 18.0 |
| Example 94 | 62.5 |
| Example 95 | 575.8 |
| Example 96 | 35.8 |
| Example 97 | 13.7 |
| Example 98 | 8.5 |
| Example 99 | 9.6 |
| Example 100 | 154.7 |
| Example 101 | 11.6 |
| Example 102 | 73.0 |
| Example 103 | 83.0 |
| Example 104 | 74.2 |
| Example 107 | 405.4 |
| Example 109 | 63.8 |
| Example 110 | 155.4 |
| Example 111 | 15.8 |
| Example 112 | 72.2 |
| Example 113 | 58.0 |
| Example 114 | 34.6 |
| Example 115 | 46.5 |
| Example 116 | 95.9 |
| Example 117 | 37.0 |
| Example 118 | 79.2 |
| Example 119 | 67.9 |
| Example 120 | 94.9 |
| Example 122 | 47.9 |
| Example 123 | 21.2 |
| Example 124 | 19.7 |
| Example 125 | 97.2 |
| Example 127 | 22.2 |
| Example 128 | 278.2 |
| Example 130 | 442.2 |
| Example 131 | 5.2 |
| Example 132 | 4.0 |
| Example 133 | 4.1 |
| Example 140 | 7.3 |
| Example 141 | 80.6 |
| Example 142 | 350.8 |
| Example 144 | 47.8 |
| Example 145 | 36.2 |
| Example 146 | 82.1 |
| Example 147 | 790.3 |
| Example 148 | 637.8 |
| Example 149 | 90.7 |
| Example 150 | 16.4 |
| Example 151 | 80.3 |
| Example 152 | 29.8 |
| Example 153 | 59.8 |
| Example 154 | 16.7 |
| Example 155 | 40.3 |
| Example 156 | 29.7 |
| Example 157 | 128.3 |
| Example 158 | 55.2 |
| Example 159 | 171.7 |
| Example 160 | 13.0 |
| Example 161 | 43.4 |
| Example 162 | 141.4 |
| Example 163 | 189.9 |
| Example 164 | 156.4 |
| Example 165 | 36.5 |
| Example 166 | 344.0 |
| Example 167 | 36.5 |
| Example 168 | 31.2 |
| Example 169 | 5.7 |
| Example 170 | 61.6 |
| Example 171 | 5.7 |
| Example 172 | 68.1 |
| Example 173 | 6.8 |
| Example 174 | 15.8 |
| Example 175 | 22.3 |
| Example 176 | 27.0 |
| Example 177 | 36.1 |
| Example 178 | 25.3 |
| Example 179 | 276.4 |
| Example 180 | 114.0 |
| Example 181 | 49.7 |
| Example 182 | 228.1 |
| Example 184 | 73.8 |
| Example 185 | 69.4 |
| Example 186 | 25.4 |
| Example 187 | 139.7 |
| Example 188 | 529.6 |
| Example 190 | 111.7 |
| Example 191 | 20.2 |
| Example 192 | 782.4 |
| Example 193 | 52.7 |
| Example 194 | 728.3 |
| Example 195 | 43.9 |
| Example 196 | 186.1 |
| Example 197 | 14.0 |
| Example 198 | 12.2 |
| Example 199 | 12.0 |
| Example 200 | 10.9 |
| Example 201 | 7.2 |
| Example 202 | 5.2 |
| Example 203 | 8.5 |
| Example 204 | 25.2 |
| Example 205 | 14.3 |
| Example 206 | 8.0 |
| Example 207 | 40.0 |
| Example 208 | 1059 |
| Example 210 | 53.4 |
| Example 211 | 12.8 |
| Example 212 | 15.2 |
| Example 213 | 14.3 |
| Example 214 | 12.6 |
| Example 215 | 10.0 |
| Example 216 | 56.0 |
| Example 217 | 27.0 |
| Example 218 | 25.6 |
| Example 219 | 7.3 |
| Example 220 | 61.5 |
| Example 221 | 42.9 |
| Example 222 | 35.1 |
| Example 223 | 135.4 |
| Example 224 | 88.1 |
| Example 225 | 13.5 |
| Example 226 | 248.2 |
| Example 227 | 83.5 |
| Example 228 | 13.2 |
| Example 229 | 24.0 |
| Example 230 | 6.9 |
| Example 231 | 3.7 |
| Example 232 | 8.7 |
| Example 233 | 5.0 |
| Example 234 | 5.5 |
| Example 236 | 19.5 |
| Example 237 | 58.6 |
| Example 238 | 65.8 |
| Example 239 | 55.0 |
| Example 240 | 48.7 |
| Example 243 | 719.6 |
| Example 245 | 496.9 |
| Example 248 | 115.4 |
| / | / |

TABLE 7
Inhibitory activity of compounds
on MIA Paca-2 cells

| Example | MIA PaCa-2 |
|---|---|
| Number | (IC₅₀, nM) |
| Example 1 | 98 |
| Example 2 | 485 |
| Example 4 | 494 |
| Example 6 | 17.1 |
| Example 7 | 32.3 |
| Example 8 | 73.6 |
| Example 10 | 83.2 |
| Example 13 | 144.4 |
| Example 15 | 71.1 |
| Example 17 | 87.9 |
| Example 20 | 64.1 |
| Example 22 | 92.1 |
| Example 34 | 254.1 |
| Example 38 | 59.8 |
| Example 42 | 283.5 |
| Example 46 | 91.9 |
| Example 49 | 110 |
| Example 51 | 50.7 |
| Example 58 | 26.2 |
| Example 70 | 40.1 |
| Example 84 | 245.0 |
| Example 88 | 333.1 |
| Example 89 | 510.7 |
| Example 90 | 51.9 |
| Example 91 | 30.7 |
| Example 92 | 31.0 |
| Example 93 | 39.4 |
| Example 94 | 65.9 |
| Example 96 | 90.0 |
| Example 97 | 109.2 |
| Example 99 | 16.7 |
| Example 101 | 11.6 |
| Example 102 | 142.5 |
| Example 103 | 344.5 |
| Example 104 | 138.7 |
| Example 109 | 94.2 |
| Example 111 | 44.4 |
| Example 116 | 106.7 |
| Example 122 | 105.8 |
| Example 123 | 84.1 |
| Example 124 | 58.4 |
| Example 131 | 25.0 |
| Example 132 | 16.1 |
| Example 133 | 12.0 |
| Example 134 | 19.6 |
| Example 135 | 9.1 |
| Example 136 | 6.6 |
| Example 137 | 6.8 |
| Example 138 | 10.5 |
| Example 144 | 58.7 |
| Example 145 | 77.2 |
| Example 150 | 58.5 |
| Example 152 | 68.8 |
| Example 153 | 56.7 |
| Example 154 | 28.8 |
| Example 155 | 77.6 |
| Example 158 | 139.8 |
| Example 159 | 357.4 |
| Example 160 | 48.4 |
| Example 161 | 67.8 |
| Example 165 | 250.5 |
| Example 166 | 368.9 |
| Example 167 | 111.7 |
| Example 168 | 48.5 |
| Example 169 | 31.7 |
| Example 170 | 118.0 |
| Example 171 | 12.6 |
| Example 172 | 171.2 |
| Example 173 | 9.2 |
| Example 174 | 15.6 |
| Example 175 | 22.8 |
| Example 176 | 29.3 |
| Example 177 | 25.9 |
| Example 178 | 43.1 |
| Example 179 | 87.8 |
| Example 180 | 45.2 |
| Example 181 | 56.2 |
| Example 182 | 201.6 |
| Example 184 | 60.2 |
| Example 185 | 103.6 |
| Example 186 | 52.9 |
| Example 187 | 173.3 |
| Example 188 | 865.3 |
| Example 190 | 108.2 |
| Example 191 | 54.22 |
| Example 192 | 827.1 |
| Example 193 | 114.9 |
| Example 194 | 782.1 |
| Example 195 | 93.3 |
| Example 196 | 154.6 |
| Example 197 | 21.3 |
| Example 198 | 8.7 |
| Example 199 | 41.1 |
| Example 200 | 40.5 |
| Example 201 | 31.9 |
| Example 202 | 11.6 |
| Example 203 | 51.4 |
| Example 204 | 114.8 |
| Example 205 | 78.9 |
| Example 206 | 21.0 |
| Example 207 | 73.3 |
| Example 208 | 1035 |
| Example 210 | 47.7 |
| Example 211 | 10.4 |
| Example 212 | 14.0 |
| Example 213 | 16.4 |
| Example 214 | 6.3 |
| Example 215 | 6.3 |
| Example 216 | 47.7 |
| Example 217 | 21.1 |
| Example 218 | 21.5 |
| Example 219 | 5.8 |
| Example 220 | 58.5 |
| Example 221 | 60.0 |
| Example 222 | 29.1 |
| Example 223 | 94.4 |
| Example 224 | 79.3 |
| Example 225 | 15.6 |
| Example 226 | 357.4 |
| Example 227 | 76.2 |
| Example 228 | 34.0 |
| Example 229 | 40.8 |
| Example 230 | 24.2 |
| Example 231 | 13.2 |
| Example 232 | 17.7 |
| Example 233 | 14.2 |
| Example 234 | 14.9 |
| Example 236 | 34.6 |
| Example 237 | 71.6 |
| Example 238 | 79.5 |
| Example 239 | 70.2 |
| Example 240 | 55.2 |
| Example 243 | 392.7 |
| Example 245 | 826.9 |
| Example 248 | 44.1 |
| / | / |

From the data in Tables 6-7, it can be seen that the compounds of the present disclosure have strong anti-cell proliferation activity against KRAS G12C mutated tumor cells.

### Test Example 6t Generation of NCI-H358-AMGR Drug-Resistant Cell Line

NCI-H358 cells were seeded and cultured at 50 to 70% confluence in six-well plate with RPMI-640 (10% FBS) medium at 37° C. with 5%4 CO₂. AMG510 was added for induction at three starting concentrations of 1 nM, 10 nM and 50 nM, and the medium containing the same concentration of AMG510 was replaced every 3 days. Cells were passaged once they reached confluence. After every, two passages at a given concentration of drug, the concentration was increased in a 3-old increment until final concentrations of 1 μM for AMG510 was achieved. The inhibitory effects of AMG510 on both drug-resistant and parental cell lines were compared, and successful generation of the NCI-H358-AMGR drug-resistant cell line was confirmed when the inhibition rate at the maximum dose (10 μM) was ≤50%.

NCI-H358-AMGR cells in logarithmic growth phase were collected and seeded into a clear-bottom 96-well plate at a density of 4×10³ cells per well in 80 μL of medium, and then incubated overnight at 37° C. with 5% CO₂. The compounds were serially diluted 5-fold in DMSO to obtain 8 concentrations, and then further diluted with RPMI-640) (10% FBS) cell culture medium to obtain the working solutions (5×), 20 μL of each working solution was added to the cell culture supernatant in each well, and the plate was further incubated for 3 days at 37° C. with 5% CO₂. The plate was then equilibrated with CellTiter-Glo® reagent at room temperature (25° C.) for 10-30 minutes. After aspirating 50 μL of the medium, 50 μL of CellTiter-Glo®, reagent was added. The cells were thoroughly mixed with the CellTiter-Glo®, reagent using a microplate shaker for 2 minutes, and then incubated at room temperature for 10 minutes. The %-well plate was placed in a multi-mode microplate reader to record the luminescence values (RLU).

The inhibitory rate was calculated using the following formula:

$$\% Inhibition rate = ⌊ \frac{{\overline{Ratio}}_{Negative reference} - {\overline{Ratio}}_{Tested compound}}{{\overline{Ratio}}_{Negative reference}} ⌋ * 100$$

Data were analyzed using GraphPad Prism for IC₅₀ values (Note: The negative control refers to the group without inhibitor). The data were presented in Table 8.

TABLE 8
NCI-H358-AMGR drug-resistant cell
proliferation inhibitory activity test

| | NCI-H358-AMGR |
|---|---|
| | Proliferation |
| Compound | IC₅₀(nM) |
| AMG-510 | >10000 |
| Example 1 | 26.96 |
| Example 6 | 15.58 |
| Example 7 | 41.69 |
| Example 8 | 81.6 |
| Example 10 | 86.6 |
| Example 15 | 62.1 |
| Example 17 | 53.3 |
| Example 20 | 48.7 |
| Example 22 | 47.9 |
| Example 38 | 51.1 |
| Example 42 | 132.3 |
| Example 46 | 27.8 |
| Example 49 | 45.5 |
| Example 51 | 61.0 |
| Example 56 | 116.5 |
| Example 58 | 100.1 |
| Example 70 | 74.5 |
| Example 84 | 639.4 |
| Example 90 | 56.2 |
| Example 91 | 83.9 |
| Example 92 | 110.7 |
| Example 93 | 69.3 |
| Example 94 | 65.2 |
| Example 99 | 65.2 |
| Example 101 | 28.3 |
| Example 102 | 516.0 |
| Example 109 | 112.8 |
| Example 111 | 117.8 |
| Example 116 | 232.2 |
| Example 122 | 127.6 |
| Example 123 | 117.9 |
| Example 124 | 85.5 |
| Example 131 | 19.1 |
| Example 144 | 43.1 |
| Example 145 | 223.4 |
| Example 150 | 112.0 |
| Example 152 | 62.1 |
| Example 153 | 100.2 |
| Example 154 | 61.6 |
| Example 155 | 163.5 |
| Example 158 | 154.7 |
| Example 159 | 360.7 |
| Example 160 | 69.5 |
| Example 161 | 145.4 |
| Example 165 | 170.8 |
| Example 167 | 73.0 |
| Example 168 | 239.5 |
| Example 169 | 36.2 |
| Example 170 | 379.5 |
| Example 171 | 41.5 |
| Example 172 | 450.7 |
| Example 173 | 54.7 |
| Example 174 | 28.6 |
| Example 175 | 58.1 |
| Example 176 | 76.1 |
| Example 177 | 48.9 |
| Example 178 | 66.2 |
| Example 181 | 283.5 |
| Example 184 | 134.7 |
| Example 185 | 131.1 |
| Example 186 | 43.9 |
| Example 187 | 372.6 |
| Example 191 | 36.4 |
| Example 193 | 21.6 |
| Example 195 | 92.3 |
| Example 197 | 38.4 |
| Example 198 | 22.1 |
| Example 199 | 21.7 |
| Example 200 | 6.5 |
| Example 201 | 5.3 |
| Example 202 | 9.6 |
| Example 203 | 5.4 |
| Example 204 | 47.1 |
| Example 205 | 15.2 |
| Example 206 | 9.5 |
| Example 210 | 286.9 |
| Example 217 | 32.1 |
| Example 218 | 13.5 |
| Example 219 | 15.9 |
| Example 220 | 206.8 |
| Example 223 | 85.2 |
| Example 224 | 219.5 |
| Example 225 | 34.9 |
| Example 226 | 358.8 |
| Example 228 | 40.3 |
| Example 229 | 30.0 |
| Example 230 | 26.7 |
| Example 231 | 21.6 |
| Example 232 | 44.0 |
| Example 233 | 52.0 |
| Example 234 | 58.2 |
| Example 236 | 44.7 |

### Test Example 7: Generation of MIA PaCa-2-AMGR drug-resistant cell line

MIA PaCa-2 cells were seeded and cultured at 50 to 70% confluence in six-well plate with DMEM (10% FBS+2.5% horse serum) medium at 37° C. with 5% CO₂. AMG510 was added for induction at three starting concentrations of 1 nM, 10 nM and 50 nM, and the medium containing the same concentration of AMG510 was replaced every 3 days. Cells were passaged once they reached confluence. After every two passages at a given concentration of drug, the concentration was increased in a 3-fold increment until final concentrations of 1 μM for AMG510 was achieved. The inhibitory effects of AMG510 on both drug-resistant and parental cell lines were compared, and successful generation of the MIA PaCa-2-AMGR drug-resistant cell line was confirmed when the inhibition rate at the maximum dose (10 μM) was <50%.

MIA PaCa-2-AMGR cells in logarithmic growth phase were collected and seeded into a clear-bottom 96-well plate at a density of 4×10³ cells per well in 80 μL of medium, and then incubated overnight at 37° C. with 5% CO₂. The compounds were serially diluted 5-fold in DMSO to obtain 8 concentrations, and then further diluted with DMEM (10% FBS+2.5% horse serum) cell culture medium to obtain the working solutions (5×), 20 μL of each working solution was added to the cell culture supernatant in each well, and the plate was further incubated for 3 days at 37° C. with 5% CO₂. The plate was then equilibrated with CellTiter-Glo® reagent at room temperature (25° C.) for 10-30 minutes. After aspirating 50 μL of the medium, 50 μL of CellTiter-Glo® reagent was added. The cells were thoroughly mixed with the CellTiter-Glo® reagent using a microplate shaker for 2 minutes, and then incubated at room temperature for 10 minutes. The 96-well plate was placed in a multi-mode microplate reader to record the luminescence values (RLU).

The inhibitory rate was calculated using the following formula:

$$\% Inhibition rate = ⌊ \frac{{\overline{Ratio}}_{Negative reference} - {\overline{Ratio}}_{Tested compound}}{{\overline{Ratio}}_{Negative reference}} ⌋ * 100$$

Data were analyzed using GraphPad Prism for IC₅₀ values (Note: The negative control refers to the group without inhibitor) The data were presented m Table 9

TABLE 9
MIA PaCa2-AMGR drug-resistant cell
proliferation inhibitory activity test

| | MIA PaCa-2~ |
|---|---|
| | AMGR |
| | proliferation |
| | inhibitory |
| Compound | (IC₅₀, nM) |
| AMG-510 | >10000 |
| Example 1 | 36.0 |
| Example 6 | 49.2 |
| Example 7 | 222.4 |
| Example 8 | 249.5 |
| Example 10 | 225.8 |
| Example 13 | 433.1 |
| Example 15 | 243.2 |
| Example 17 | 106 |
| Example 20 | 111.4 |
| Example 22 | 76.7 |
| Example 38 | 215.5 |
| Example 42 | 185.4 |
| Example 46 | 77.4 |
| Example 49 | 62.4 |
| Example 51 | 181.3 |
| Example 56 | 115.5 |
| Example 59 | 141.4 |
| Example 70 | 139.2 |
| Example 90 | 105.9 |
| Example 91 | 79.8 |
| Example 92 | 56.9 |
| Example 93 | 114.0 |
| Example 94 | 126.4 |
| Example 99 | 72.9 |
| Example 101 | 35.9 |
| Example 102 | 908.0 |
| Example 109 | 110.9 |
| Example 111 | 90.1 |
| Example 116 | 462.3 |
| Example 122 | 273.0 |
| Example 123 | 328.0 |
| Example 124 | 83.9 |
| Example 131 | 29.6 |
| Example 144 | 56.6 |
| Example 145 | 112.9 |
| Example 150 | 55.1 |
| Example 152 | 52.1 |
| Example 153 | 49.7 |
| Example 154 | 32.2 |
| Example 155 | 111.9 |
| Example 158 | 135.6 |
| Example 159 | 226.4 |
| Example 160 | 53.8 |
| Example 161 | 55.3 |
| Example 165 | 173.1 |
| Example 195 | 280.0 |
| Example 168 | 191.5 |
| Example 169 | 54.4 |
| Example 170 | 182.7 |
| Example 171 | 32.9 |
| Example 172 | 256.3 |
| Example 173 | 62.6 |
| Example 174 | 26.3 |
| Example 175 | 36.7 |
| Example 176 | 48.6 |
| Example 177 | 36.6 |
| Example 178 | 34.0 |
| Example 181 | 117.9 |
| Example 184 | 175.5 |
| Example 185 | 45.7 |
| Example 186 | 31.3 |
| Example 187 | 223.7 |
| Example 191 | 40.8 |
| Example 193 | 66.2 |
| Example 195 | 81.1 |
| Example 197 | 15.6 |
| Example 198 | 10.7 |
| Example 199 | 19.1 |
| Example 200 | 4.3 |
| Example 201 | 11.1 |
| Example 202 | 13.0 |
| Example 203 | 16.7 |
| Example 204 | 45.8 |
| Example 205 | 32.9 |
| Example 206 | 6.9 |
| Example 210 | 97.4 |
| Example 217 | 21.4 |
| Example 218 | 16.1 |
| Example 219 | 6.4 |
| Example 220 | 212.1 |
| Example 223 | 63.1 |
| Example 224 | 144.1 |
| Example 225 | 26.0 |
| Example 226 | 424.5 |
| Example 228 | 13.5 |
| Example 229 | 31.3 |
| Example 230 | 35.3 |
| Example 231 | 3.6 |
| Example 232 | 9.0 |
| Example 233 | 16.7 |
| Example 234 | 5.3 |
| Example 236 | 28.8 |

From the data in Table 9, it can be observed that the compounds of the present disclosure exhibit potent inhibitory effects against KRAS G12C inhibitor-resistant tumor cell lines (e.g., resistant to AMG-510) and can be used to overcome drug resistance to KRAS G12C inhibitors.

Although the embodiments of the present disclosure have been shown and described, it will be understood by those of ordinary skill in the art that various changes, modifications, substitutions and variations can be made to these embodiments without departing from the principles and spirit of the disclosure. The scope of the disclosure is defined by the appended claims and their equivalents.

What is claimed is:

1. A compound of formula I″″, or a tautomer, meso isomer, racemate, enantiomer, diastereomer or mixture thereof, or a pharmaceutically acceptable hydrate, solvate or salt thereof: 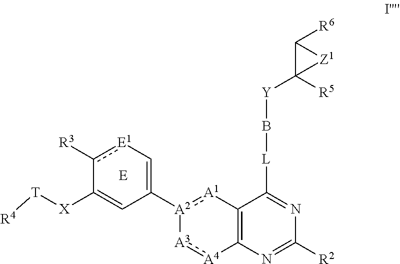
wherein,
T is selected from S(O)₂, C═O, CH₂ or NHS(O)₂;
X is selected from NR¹, S or S(O)₂;
A¹ is selected from CH, CH₂, C═O or N, A² is selected from C, CH or N, A³ is selected from CR⁸, CR⁹R¹⁰ or N, A⁴ is selected from CR²⁹, CR⁹R¹⁰ or N;
Z¹ is a bond or O;
E¹ is NR¹¹, CH or CH₂;
B is 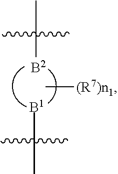
the ring where B¹, B² are located is a 5- to 10-membered nitrogen-containing heterocycloalkyl or a 5- to 10-membered nitrogen-containing heterocycloalkenyl, wherein the cycloalkyl or cycloalkenyl is a monocyclic ring, spirocyclic ring or bridged cyclic ring, B¹ is selected from CR¹² or N, B² is selected from CR¹³ or N, and at least one of B¹ and B² is N;
or, B is 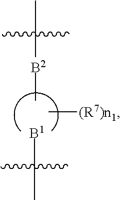
the ring where B¹ is located is a 4- to 8-membered nitrogen-containing monoheterocycloalkyl, B¹ is selected from CR¹² or N, B² is selected from CR¹³R^{13′} or NR¹⁴;
or, B is 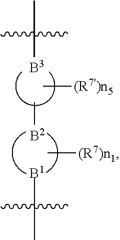
the ring where B¹, B² are located is a 5- to 10-membered nitrogen-containing heterocycloalkyl and is a monocyclic ring, spirocyclic ring or bridged cyclic ring, B¹ is selected from CR¹² or N, B² is selected from CR^{13″} or N, and at least one of B¹ and B² is N; the ring where B³ is located is a 4- to 10-membered nitrogen-containing heterocycloalkyl and is a monocyclic ring, spirocyclic ring or bridged cyclic ring, B³ is selected from CR^{12′} or N;
B¹ is connected to L;
L is selected from a bond or NR¹⁵;
Y is selected from C═O or S(O)₂;
R⁵, R⁶ are independently selected from hydrogen, halogen, cyano, alkyl, aliphatic heterocyclyl, aryl, heteroaryl, —OR^{a}, —NR^{b}R^{c}, —C(O)R¹⁶, —S(O)R¹⁶, —S(O)₂R¹⁶, —P(O)R¹⁶R¹⁷, —C(O)NR^{b}R^{c} or —C(O)OR^{a}, wherein the alkyl, aliphatic heterocyclyl, aryl, heteroaryl are optionally substituted by one or more R¹⁸;
each occurrence of R¹⁸ is independently selected from halogen, cyano, alkyl, cycloalkyl, aliphatic heterocyclyl, —OR^{a}, —NR^{b}R^{c}, —C(O)R¹⁶, —C(O)NR^{b}R^{c} or —C(O)OR^{a};
R¹, R¹¹, R¹⁴, R¹⁵ are independently selected from hydrogen, alkyl, cycloalkyl, aliphatic heterocyclyl or —C(O)R¹⁹, wherein the alkyl, cycloalkyl, aliphatic heterocyclyl are optionally substituted by one or more substituents selected from the group consisting of: halogen, cyano, hydroxy, amino, alkyl, cycloalkyl and aliphatic heterocyclyl;
n₁, n₅ are independently selected from 0, 1, 2, 3 or 4;
each occurrence of R¹², R^{12′}, R¹³, R^{13′}, R^{13″}, R⁷, R^{7′} is independently selected from hydrogen, halogen, cyano, alkyl, cycloalkyl, aliphatic heterocyclyl, aryl, heteroaryl, —ORⁿ, —NR^{o}R^{p}, —C(O)R²⁰, —C(O)NR^{o}R^{p} or —C(O)ORⁿ, wherein alkyl, cycloalkyl, aliphatic heterocyclyl, aryl, heteroaryl are optionally substituted by one or more R²¹;
each occurrence of R²¹ is independently selected from halogen, cyano, alkyl, cycloalkyl, aliphatic heterocyclyl, —ORⁿ, —NR^{o}R^{p}, —C(O)R²⁰, —C(O)NR^{o}R^{p} or —C(O)ORⁿ;
or Z¹ is a bond, R⁵, R⁶ together with the atoms to which R⁵, R⁶ are attached form a 4- to 9-membered cycloalkenyl, or R⁵, R¹⁴ together with the fragment to which R⁵, R¹⁴ are attached form a 4- to 9-membered aliphatic heterocyclyl, or R⁶, R¹³ together with the fragment to which R⁶, R¹³ are attached form a 4- to 9-membered cycloalkenyl, or R⁵, R¹³ together with the fragment to which R⁶, R¹³ are attached form a 4- to 9-membered alicyclyl, or R⁶, R⁴ together with the fragment to which R⁶, R¹⁴ are attached form a 4- to 9-membered aliphatic heterocyclyl, or R⁵, R⁶ together with the atoms to which R⁵, R⁶ are attached form a 6- to 10-membered aryl or 5- to 10-membered heteroaryl, wherein the cycloalkenyl, alicyclic heterocyclyl, alicyclyl, aryl and heteroaryl are optionally substituted by one or more substituents selected from the group consisting of: halogen, cyano, hydroxy, amino, alkyl, cycloalkyl and alicyclic heterocyclyl;
R² is selected from hydrogen, halogen, cyano, alkyl, cycloalkyl, alicyclic heterocyclyl, aryl, heteroaryl, —OR^{d}, —NR^{e}R^{f}, —C(O)R²², —C(O)R^{e}R^{f} or —C(O)OR^{d}, wherein the alkyl, cycloalkyl, alicyclic heterocyclyl, aryl, heteroaryl are optionally substituted by one or more R²³;
each occurrence of R²³ is independently selected from halogen, cyano, alkyl, cycloalkyl, aliphatic heterocyclyl, —OR^{d}, —NR^{e}R^{f}, —C(O)R²², —C(O)NR^{e}R^{f} or —C(O)OR^{d};
R³ is selected from hydrogen, halogen, cyano, alkyl, cycloalkyl, aliphatic heterocyclyl, aryl, heteroaryl, —OR⁹, —SR^{g}, —NR^{h}Rⁱ, —C(O)R²⁴, —C(O)R^{h}Rⁱ or —C(O)OR^{g}, when the ‘ [symbol] ’ in ring E is absent, R³ can also be a carbonyl group that together formed by the connected carbon atom, wherein alkyl, cycloalkyl, aliphatic heterocyclyl, aryl, heteroaryl are optionally substituted by one or more R²⁵;
each occurrence of R²⁵ is independently selected from halogen, cyano, alkyl, cycloalkyl, aliphatic heterocyclyl, —OR^{g}, —NR^{h}Rⁱ, —C(O)R²⁴, —C(O)NR^{h}Rⁱ or —C(O)OR^{g};
R⁴ is selected from alkyl, cycloalkyl, aliphatic heterocyclyl, aryl or heteroaryl, wherein the alkyl, cycloalkyl, aliphatic heterocyclyl, aryl, heteroaryl are optionally substituted by one or more R²⁶;
each occurrence of R²⁶ is independently selected from halogen, cyano, alkyl, cycloalkyl, aliphatic heterocyclyl, aryl, heteroaryl, —OR^{j}, —NR^{k}R^{m}, —C(O)R²⁷, —C(O)NR^{k}R^{m} or —C(O)OR^{j}, wherein the alkyl, cycloalkyl, aliphatic heterocyclyl, aryl, heteroaryl are optionally substituted by one or more R²⁸;
each occurrence of R²⁸ is independently selected from halogen, cyano, alkyl, cycloalkyl, aliphatic heterocyclyl, —OR^{j}, —NR^{k}R^{m}, —C(O)R²⁷, —C(O)NR^{k}R^{m} or —C(O)OR^{j};
each occurrence of R⁸, R⁹, R¹⁰, R²⁹ is independently selected from hydrogen, halogen, cyano, alkyl, cycloalkyl, aliphatic heterocyclyl, aryl, heteroaryl, —OR^{q}, —NR^{r}R^{s}, —C(O)R³⁰, —C(O)NR^{r}R^{s} or —C(O)OR^{q}; wherein the alkyl, cycloalkyl, aliphatic heterocyclyl, aryl, heteroaryl are optionally substituted by one or more R³¹;
When the ‘ [symbol] ’ in ring E is a bond, R¹¹ is absent;
each occurrence of R³¹ is independently selected from halogen, cyano, alkyl, cycloalkyl, aliphatic heterocyclyl, —OR^{q}, —NR^{r}R^{s}, —C(O)R³⁰, —C(O)NR^{r}R^{s} or —C(O)OR^{q};
each occurrence of R^{a}, R^{b}, R^{c}, R^{d}, R^{e}, R^{f}, R^{g}, R^{h}, Rⁱ, R^{j}, R^{k}, R^{m}, Rⁿ, R^{o}, R^{p}, R^{q}, R^{r}, R^{s} is independently selected from H, alkyl, cycloalkyl, aliphatic heterocyclyl or —C(O)R³², wherein the alkyl, cycloalkyl, alicyclic heterocyclyl are optionally substituted by one or more substituents selected from the group consisting of: halogen, cyano, hydroxy, amino, alkyl, cycloalkyl and alkyl substituted or unsubstituted aliphatic heterocyclyl;
each occurrence of R¹⁶, R¹⁷, R¹⁹, R²⁰, R²², R²⁴, R²⁷, R³⁰, R³² is independently selected from H, alkyl, cycloalkyl or alicyclic heterocyclyl, wherein the alkyl, cycloalkyl, alicyclic heterocyclyl are optionally substituted by one or more substituents selected from the group consisting of: halogen, cyano, hydroxy, amino, alkyl, cycloalkyl and aliphatic heterocyclyl.

2. (canceled)

3. The compound according to claim 1, wherein 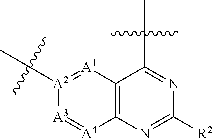
is selected from the following groups: 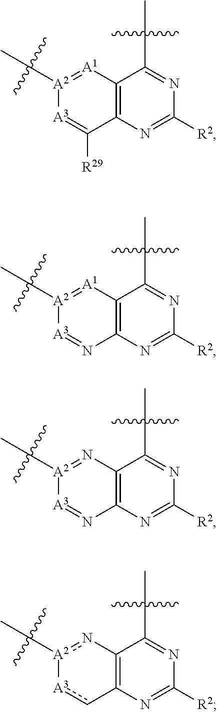
preferably, 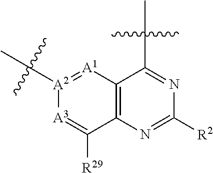
is selected from the following groups: 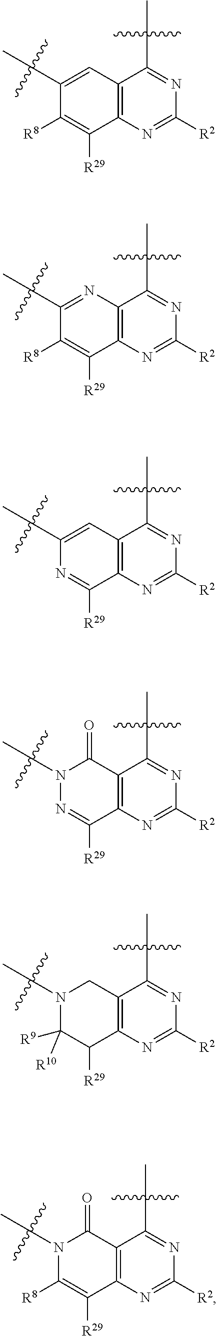
preferably 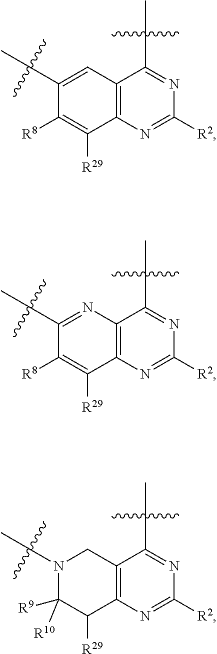
more preferably 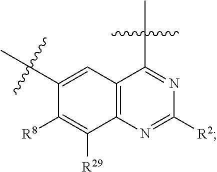
preferably, R⁸, R⁹, R¹⁰, R²⁹ are independently selected from hydrogen, halogen, cyano, C1˜C3 alkyl or —OR^{q}, wherein the alkyl is optionally substituted by one or more R³¹;
each occurrence of R³¹ is independently selected from halogen, C1˜C3 alkyl or —OR^{q};
preferably, R⁸, R²⁹ are independently selected from hydrogen, F, Cl, Br, C1˜C3 alkyl, halogenated C1˜C3 alkyl, —CN or —OR^{q}, R⁹, R¹⁰ is hydrogen;
preferably, each occurrence of R^{q} is independently selected from H, C1˜C6 alkyl or C3˜C6 cycloalkyl, wherein the alkyl, cycloalkyl are optionally substituted by one or more of the following substituents: halogen, cyano, hydroxy, amino, C1˜C6 alkyl; preferably, each occurrence of R^{q} is independently selected from H or C1˜C3 alkyl;
more preferably, R⁸ is selected from hydrogen, Cl or —CH₃; R²⁹ is selected from hydrogen, OH, —CN, F or —CF₃.

4. (canceled)

5. (canceled)

6. (canceled)

7. (canceled)

8. (canceled)

9. The compound according to claim 1, wherein
B is 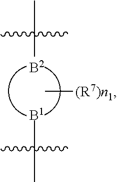
the ring where B¹, B² are located is a 5- to 6-membered nitrogen-containing monoheterocycloalkyl, 6-membered nitrogen-containing monoheterocycloalkenyl or 8- to 9-membered nitrogen-containing spiroheterocycloalkyl;
or B is 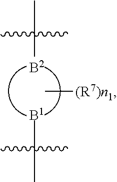
the ring where B¹, B² are located is a 6- to 9-membered nitrogen-containing bridged heterocycloalkyl, preferably 7- to 8-membered nitrogen-containing bridged heterocycloalkyl, more preferably 8-membered nitrogen-containing bridged heterocycloalkyl;
or B is 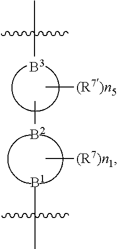
the ring where B¹, B² are located is a 6- to 7-membered nitrogen-containing monoheterocycloalkyl; the ring where B³ is located is a 4- to 6-membered nitrogen-containing monoheterocycloalkyl;
further, 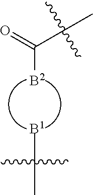
is selected from the following groups: 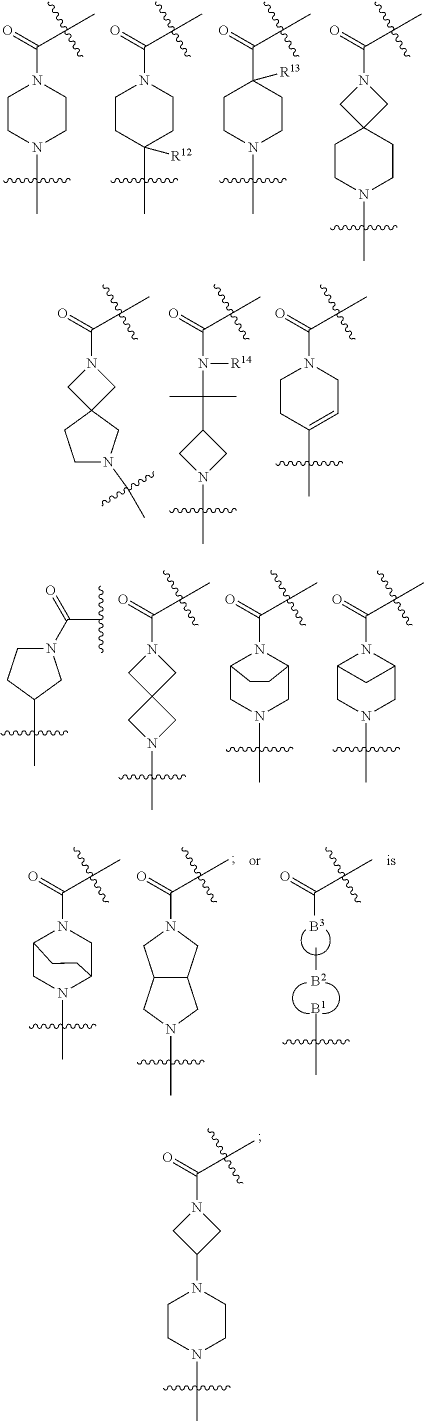
R¹² is selected from hydrogen, halogen or C1˜C6 alkyl, preferably hydrogen, halogen or C1˜C3 alkyl, more preferably hydrogen;
R¹³ is selected from hydrogen, halogen or C1˜C6 alkyl, preferably hydrogen, halogen or C1˜C3 alkyl;
further, 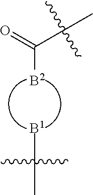
is selected from 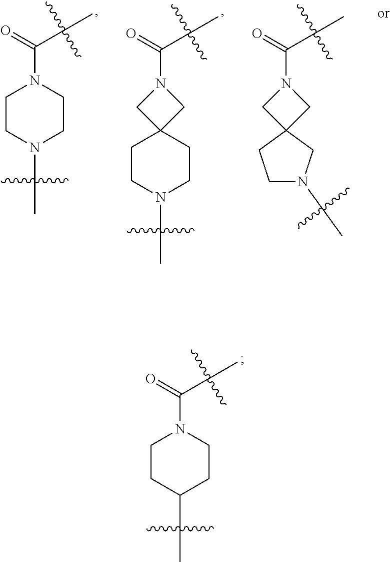 [SMILES: *C(=O)N1CCN(*)CC1 ; *C(=O)N1CC2(CCN(*)CC2)C1 ; *C(=O)N1CC2(CCN(*)C2)C1 ; *C(=O)N1CCC(*)CC1]
preferably 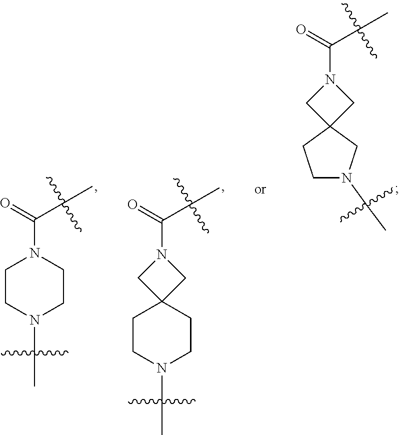 [SMILES: *C(=O)N1CCN(*)CC1 ; *C(=O)N1CC2(CCN(*)CC2)C1 ; *C(=O)N1CC2(CCN(*)C2)C1]
more preferably, 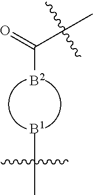
is

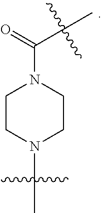 [SMILES: *C(=O)N1CCN(*)CC1]

10. (canceled)

11. The compound according to claim 1, wherein the compound has a structure shown in formula II, or a tautomer, meso isomer, racemate, enantiomer, diastereomer or mixture thereof, or a pharmaceutically acceptable hydrate, solvate or salt thereof: 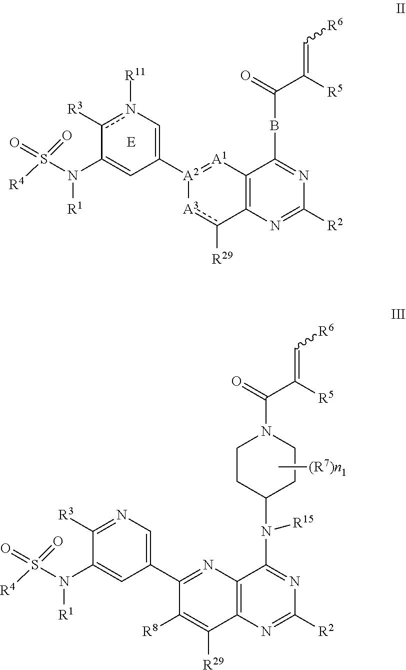
in formula II, 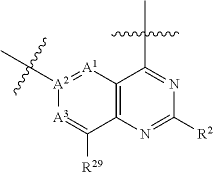
is not 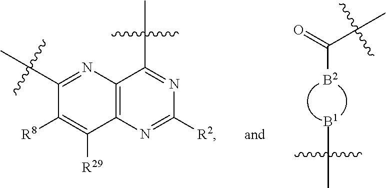
is not 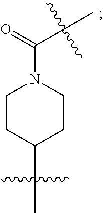 [SMILES: *C(=O)N1CCC(*)CC1]
preferably in formula II, when 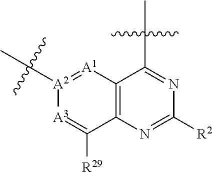
is 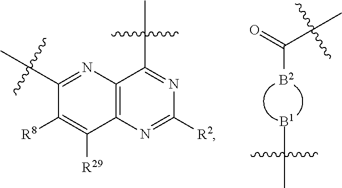
preferably is

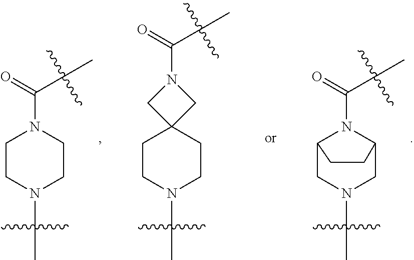 [SMILES: *C(=O)N1CCN(*)CC1 ; *C(=O)N1CC2(CCN(*)CC2)C1 ; *C(=O)N1C2CCC1CN(*)C2]

12. (canceled)

13. (canceled)

14. The compound according to any one of claim 1, wherein R¹, R¹¹ are independently selected from hydrogen, halogen, C1˜C6 alkyl or C3˜C6 cycloalkyl, wherein the alkyl, cycloalkyl are optionally substituted by one or more substituents selected from the group consisting of: halogen, cyano, hydroxy, amino, C1˜C6 alkyl, C3˜C6 cycloalkyl and 3- to 6-membered aliphatic heterocyclyl; when the ‘ [symbol] ’ in ring E is a bond, R¹¹ is absent;
further, R¹, R¹¹ are independently selected from hydrogen, halogen, C1˜C3 alkyl, wherein the alkyl is optionally substituted by one or more substituents selected from the group consisting of: halogen, cyano, hydroxy, amino, C1˜C3 alkyl and C3˜C6 cycloalkyl;
further, R¹ is selected from hydrogen or methyl, preferably hydrogen;
R¹¹ is selected from hydrogen or methyl, preferably methyl, when the ‘ [symbol] ’ in ring E is a bond, R¹¹ is absent.

15. (canceled)

16. The compound according to claim 1, wherein the compound has a structure shown in formula VII or IX, or a tautomer, meso isomer, racemate, enantiomer, diastereomer or mixture thereof, or a pharmaceutically acceptable hydrate, solvate or salt thereof: 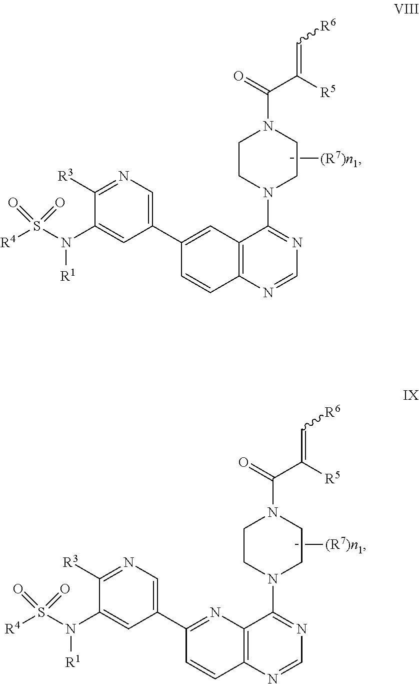
wherein n₁ is selected from 0, 1, 2 or 3, preferably 0, 1 or 2, more preferably 0 or 1.

17. The compound according to claim 1, wherein
R⁵, R⁶ are independently selected from hydrogen, halogen, C1˜C6 alkyl, C3˜C6 cycloalkyl, 3- to 6-membered aliphatic heterocyclyl, 6- to 10-membered aryl, 5- to 10-membered heteroaryl, —C(O)R¹⁶, —S(O)R¹⁶, —S(O)₂R¹⁶, —P(O)R¹⁶R¹⁷, —C(O)NR^{b}R^{c}, —C(O)OR^{a}, —OR^{a} or —NR^{b}R^{c}, wherein the alkyl, cycloalkyl, aliphatic heterocyclyl, aryl, heteroaryl are optionally substituted by one or more R¹⁸;
each occurrence of R¹⁸ is independently selected from halogen, cyano, C1˜C6 alkyl, C3˜C6 cycloalkyl, —OR^{a} or —NR^{b}R^{c};
further, R⁵, R⁶ are independently selected from hydrogen, halogen, C1˜C3 alkyl, 5- to 6-membered heteroaryl, —C(O)R¹⁶, —S(O)₂R¹⁶, —P(O)R¹⁶R¹⁷, —C(O)NR^{b}R^{c}, —C(O)OR^{a} or —OR^{a}, wherein the alkyl, heteroaryl are optionally substituted by one or more R¹⁸;
further, R⁵, R⁶ are independently selected from hydrogen, halogen, C1˜C3 alkyl, 5- to 6-membered heteroaryl, —C(O)R¹⁶, —S(O)₂R¹⁶, —P(O)R¹⁶R¹⁷, —C(O)OR^{a} or —OR^{a}, wherein the alkyl, heteroaryl are optionally substituted by one or more R¹⁸;
each occurrence of R¹⁸ is independently selected from halogen, C1˜C3 alkyl, —OR^{a} or —NR^{b}R^{c};
further, R⁵, R⁶ are independently selected from hydrogen, halogen, C1˜C3 alkyl, —C(O)R⁶, —S(O)₂R¹⁶, —P(O)R¹⁶R¹⁷, —C(O)O—C1˜C3 alkyl, —O—C1˜C3 alkyl, 1H-1,2,3-triazolyl or oxazolyl, wherein the alkyl, 1H-1,2,3-triazolyl, oxazolyl are optionally substituted by 1˜3 R¹⁸;
each occurrence of R¹⁸ is independently selected from halogen, C1˜C3 alkyl, —OR^{a} or —NR^{c};
preferably, each occurrence of R^{a}, R^{b}, R^{c} is independently selected from H or C1˜C3 alkyl, wherein the alkyl is optionally substituted by one or more of the following substituents: halogen, C1˜C3 alkyl; preferably, each occurrence of R^{a}, R^{b}, R^{c} is independently selected from H or C1˜C3 alkyl, preferably H or methyl; more preferably methyl;
preferably, R¹⁶, R¹⁷ is independently selected from H or C1˜C6 alkyl, wherein the alkyl is optional substituted by one or more substituents selected from the group consisting of: halogen, cyano, hydroxy, amino and C1˜C6 alkyl;
preferably, each occurrence of R¹⁶, R¹⁷ is independently selected from C1˜C3 alkyl, preferably methyl or propyl;
further, R⁵, R⁶ are independently selected from H, F, CN, —C(CH₃)₃, —CH(OH)CH₃, —C(O)CF₃, —C(O)CH₂CH₃, — 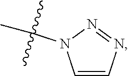 [SMILES: *n1ccnn1]
C(O)CH₃, —C(O)NHCH₃, —C(O)CH₂CH₂CH₃, —CH₂N(CH₃)₂, —CH₂F, —S(O)₂CH₂, —P(O)(CH₃)₂, —CHCH₃CH₃, —CH₂OCH₃, CF₃, 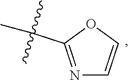 [SMILES: *c1ncco1]
—C(O)OCH₃ or —OCH₃;
further, R⁵ is selected from H, F, CN, —CH(CH₃)₂, —CH(OH)CH₃, —CH₂OCH₃, —OCH₃ or —C(O)CH₃, R⁶ is selected from H, —C(CH₃)₃, —C(O)CH₃, —C(O)CH₂CH₂CH₃, —C(O)NHCH₃, —CH₂N(CH₃)₂, —CH₂F, 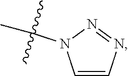 [SMILES: *n1ccnn1]
—CH₂OCH₃, —CF₃, 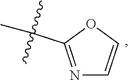 [SMILES: *c1ncco1]
—C(O)OCH₃, —P(O)(CH₃), —S(O)CH₃, —C(O)CF₃ or —C(O)CH₂CH₃;
further, Z¹ is a bond; R⁵ is H; R⁶ is selected from H, —C(CH₃)₃, —C(O)CH₃, —C(O)CH₂CH₂CH₃, —C(O)NHCH₃, —CH₂N(CH₃)₂, —CH₂F, 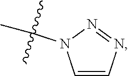 [SMILES: *n1ccnn1]
—CH₂OCH₃, CF₃, 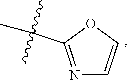 [SMILES: *c1ncco1]
—C(O)OCH₃, —P(O)(CH₃), —S(O)₂CH₃ or —C(O)CF₃; preferably, R⁶ is selected from H and —C(O)CH₃;
further preferably, 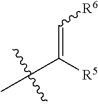
is

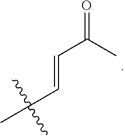 [SMILES: */C=C/C(C)=O]

18. (canceled)

19. The compound according to claim 1, wherein R² is selected from hydrogen, halogen, cyano, C1˜C6 alkyl, C3˜C6 cycloalkyl, —OR^{d} or —NR^{e}R^{f}, wherein the alkyl, cycloalkyl are optionally substituted by one or more R⁴, each occurrence of R⁴ is independently selected from halogen, cyano, C1˜C6 alkyl, C3˜C6 cycloalkyl, —OR^{d} or —NR^{e}R^{f},
further, R² is selected from hydrogen, halogen, cyano, C1˜C3 alkyl, —OR^{d} or —NR^{e}R^{f}, wherein the alkyl is optionally substituted by one or more R¹⁴, each occurrence of R¹⁴ is independently selected from halogen, cyano, C1˜C3 alkyl, —OR^{d} or —NR^{e}R^{f};
preferably, each occurrence of R^{d}, R^{e}, R^{f} is independently selected from H, C1˜C6 alkyl or C3˜C6 cycloalkyl, wherein the alkyl, cycloalkyl are optionally substituted by one or more substituents selected from the group consisting of: halogen, cyano, hydroxy, amino, C1˜C6 alkyl, C3˜C6 cycloalkyl and alkyl substituted or unsubstituted C3˜C6 heterocycloalkyl;
preferably, each occurrence of R^{d}, R^{e}, R^{f} is independently selected from H or C1˜C3 alkyl, wherein the alkyl is optionally substituted by one or more substituents selected from the group consisting of: halogen, C1˜C3 alkyl and alkyl substituted or unsubstituted 5-membered nitrogen-containing heterocycloalkyl;
further, each occurrence of R^{d}, R^{e}, R^{f} is independently selected from H or C1˜C3 alkyl, wherein the alkyl is optionally substituted by one or more of the following substituents: halogen, C1˜C3 alkyl, 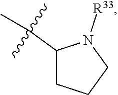
R³³ is selected from H or C1˜C6 alkyl, preferably C1˜C3 alkyl, more preferably methyl;
further, R² is hydrogen.

20. (canceled)

21. The compound according to claim 1, wherein R³ is selected from hydrogen, halogen, cyano, C1˜C6 alkyl, C3˜C6 cycloalkyl, 3- to 6-membered aliphatic heterocyclyl, —OR^{g}, —C(O)OR^{g}, —SR^{g} or —NR^{h}Rⁱ, when the ‘ [symbol] ’ in ring E is absent, R³ can also be a carbonyl group that together formed by the connected carbon atom, wherein the alkyl, cycloalkyl, aliphatic heterocyclyl are optionally substituted by one or more R²⁵, each occurrence of the R²⁵ is independently selected from halogen, cyano, C1˜C6 alkyl, C3˜C6 cycloalkyl, —OR^{g} or —NR^{h}Rⁱ;
further, R³ is selected from hydrogen, halogen, cyano, C1˜C6 alkyl, —OR^{g}, —C(O)OR^{g}, —SR^{g} or —NR^{h}Rⁱ, when the “ [symbol] ” in ring E is absent, R³ can also be a carbonyl group that together formed by the connected carbon atom, wherein the alkyl is optionally substituted by one or more halogen or —OH;
further, R³ is selected from hydrogen, halogen, C1˜C3 alkyl, —OR^{g} or —NR^{h}Rⁱ, when the ‘ [symbol] ’ in ring E is absent, R³ can also be a carbonyl group that together formed by the connected carbon atom, wherein the alkyl is optionally substituted by one or more R²³, each occurrence of R²⁵ is independently selected from halogen, cyano, C1-C3 alkyl, —OR^{g} or —NR^{h}Rⁱ;
preferably, each occurrence of R^{g}, R^{h}, Rⁱ is independently selected from H, C1˜C6 alkyl or C3˜C6 cycloalkyl, wherein the alkyl, cycloalkyl are optionally substituted by one or more substituents selected from the group consisting of: halogen, cyano, hydroxy, amino and C1˜C6 alkyl;
further, each occurrence of R^{g}, R^{h}, Rⁱ is independently selected from H, C1˜C3 alkyl, cyclopropyl or cyclopentyl, wherein the alkyl, cyclopropyl are optionally substituted by one or more of the following substituents: halogen, C1˜C3 alkyl;
further, each occurrence of R^{g}, R^{h}, Rⁱ is independently selected from H, methyl, cyclopropyl, cyclopentyl, ethyl, trifluoromethyl, trifluoroethyl, difluoromethyl or deuterated methyl;
preferably, each occurrence of R^{g}, R^{h}, Rⁱ is independently selected from H, methyl, cyclopropyl, cyclopentyl or ethyl;
further, R² is selected from hydrogen, Cl, —CF₃, —OCH₃, —OCH₂CH₃, —OCF₃, —OCHF₂, —N(CH₃)₂, —CH₃, —CH₂OH, —OCH₂CF₃, —OH, —NHCH₃, —SCH₃, —OCD₃, —CN or —C(O)OCH₃;
further, R³ is selected from hydrogen, —OCH₃, —OCH₂CH₃, —N(CH₃)₂, —CH₂OH, —OCH₂CF₃, —SCH₃, —OCD₃, —CN or —C(O)OCH₃; preferably, R³ is —OCH₃.

22. (canceled)

23. (canceled)

24. (canceled)

25. (canceled)

26. The compound according to claim 1, wherein R⁴ is selected from C1˜C6 alkyl, C3˜C9 cycloalkyl, 3- to 6-membered heterocycloalkyl, 6- to 10-membered aryl or 5- to 10-membered heteroaryl, wherein the alkyl, cycloalkyl, heterocycloalkyl, aryl, heteroaryl are optionally substituted by one or more R²⁶;
each occurrence of R²⁶ is independently selected from halogen, cyano, C1˜C6 alkyl, C3˜C6 cycloalkyl, —OR^{j}, —NR^{k}R^{m}, —C(O)R²⁷, —C(O)NR^{k}R^{m} or —C(O)OR^{j}, wherein the alkyl, cycloalkyl are optionally substituted by one or more R²⁸;
each occurrence of R²⁸ is independently selected from halogen, cyano, alkyl, cycloalkyl, —OR^{j} or —NR^{k}R^{m};
further, R⁴ is selected from C1˜C3 alkyl, C3˜C6 cycloalkyl, phenyl, naphthyl, 5- to 6-membered nitrogen-containing heteroaryl or 5- to 6-membered sulphur-containing heteroaryl, wherein the alkyl, cycloalkyl, aryl, nitrogen-containing heteroaryl, sulphur-containing heteroaryl are optionally substituted by 1-3 R²⁶;
each occurrence of R²⁶ is independently selected from halogen, cyano, C1˜C3 alkyl, halogenated C1˜C3 alkyl, —O(C1˜C3 alkyl) or —C(O)OH;
further, R⁴ is selected from C1˜C3 alkyl, cyclohexyl, phenyl, pyridyl, thienyl, naphthyl, pyrrolyl or thiazolyl, wherein the alkyl, cyclohexyl, phenyl, pyridyl, thienyl, naphthyl, pyrrolyl, thiazolyl are optionally substituted by 1˜2 R²⁶;
each occurrence of R²⁶ is independently selected from F, Cl, Br, cyano, unsubstituted C1˜C3 alkyl or C1˜C3 alkyl substituted by 1 to 3 halogen, —OR^{j}, —C(O)OR^{j}, more preferably F, Cl, methyl, methoxy, cyano, trifluoromethyl or —COOH;
preferably, each occurrence of R^{j}, R^{k}, R^{m} is independently selected from H, C1˜C6 alkyl or C3˜C6 cycloalkyl, wherein the alkyl, cycloalkyl are optionally substituted by one or more substituents selected from the group consisting of: halogen, cyano, hydroxy, amino and C1˜C6 alkyl;
further, R⁴ is selected from methyl, cyclohexyl, 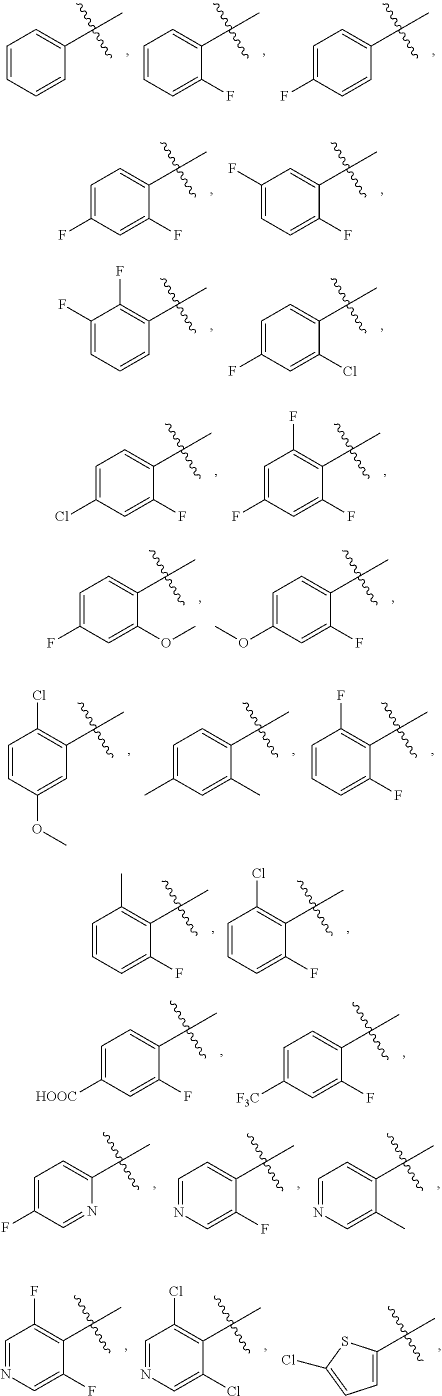 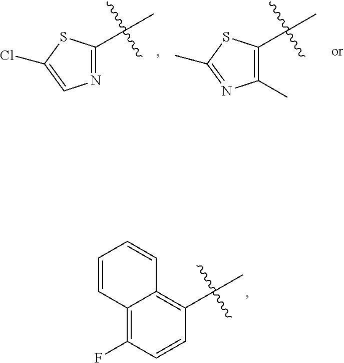
preferably methyl, cyclohexyl, 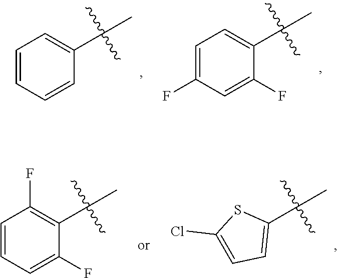 [SMILES: *c1ccccc1 ; *c1ccc(F)cc1F ; *c1c(F)cccc1F ; *c1ccc(Cl)s1]
more preferably 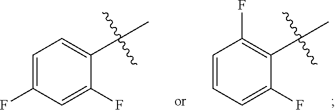 [SMILES: *c1ccc(F)cc1F ; *c1c(F)cccc1F]
or, R⁴ is selected from

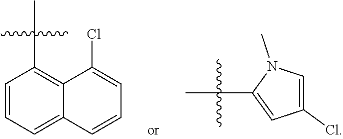

27. (canceled)

28. (canceled)

29. The compound according to claim 1, wherein each occurrence of R⁷ is independently selected from halogen, cyano, C1˜C6 alkyl, —ORⁿ, —NR^{o}R^{p}, —C(O)R²⁰, —C(O)NR^{o}R^{p} or —C(O)ORⁿ, wherein the alkyl is optionally substituted by one or more R²¹;
each occurrence of R²¹ is independently selected from halogen, cyano, C1˜C6 alkyl, —ORⁿ, —NR^{o}R^{p}, —C(O)R²⁰, —C(O)NR^{o}R^{p} or —C(O)ORⁿ,
further, each occurrence of R⁷ is independently selected from halogen, cyano, C1˜3 alkyl, —ORⁿ or —NR^{o}R, wherein the alkyl is optionally substituted by one or more R²¹;
each occurrence of R²¹ is independently selected from halogen, cyano, C1˜C3 alkyl, —ORⁿ or —NR^{o}R^{p};
preferably, each occurrence of Rⁿ, R^{o}, R^{p} is independently selected from H, C1˜C6 alkyl or C3˜C6 cycloalkyl, wherein the alkyl, cycloalkyl are optionally substituted by one or more substituents selected from the group consisting of: halogen, cyano, hydroxy, amino and C1˜C6 alkyl; preferably, each occurrence of Rⁿ, R^{o}, R^{p} is independently selected from H or C1˜C3 alkyl;
further, each occurrence of R⁷ is independently selected from C1˜C3 alkyl, wherein the alkyl is optionally substituted by one or more R²¹,
each occurrence of R²¹ is independently selected from halogen, cyano or C1˜C3 alkyl, preferably cyano;
further, R⁷ is selected from hydrogen, methyl or —CH₂CN;
further, 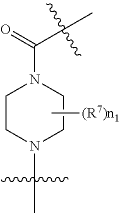
is selected from 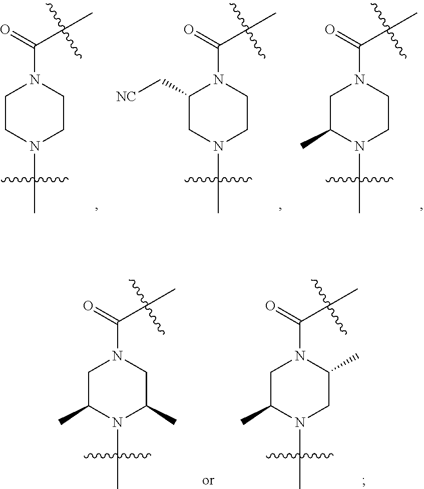
preferably, 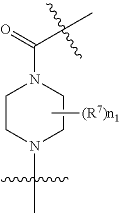
is 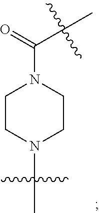 [SMILES: *C(=O)N1CCN(*)CC1]
preferably, n₁ is selected from 0, 1, 2 or 3, preferably 0, 1 or 2, more preferably 0 or 1; n₅ is selected from 0.

30. (canceled)

31. (canceled)

32. (canceled)

33. The compound according to claim 1, wherein the compound has a structure shown in formula II, or a tautomer, meso isomer, racemate, enantiomer, diastereomer or mixture thereof, or a pharmaceutically acceptable hydrate, solvate or salt thereof: 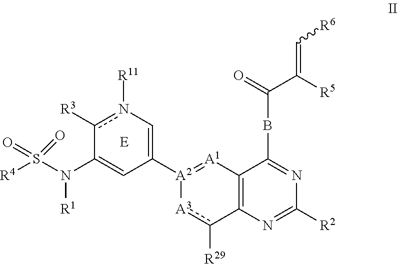
wherein 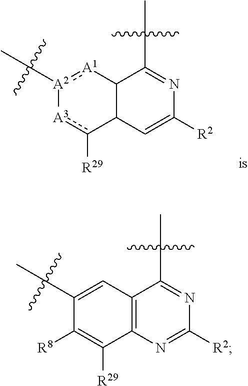
B is 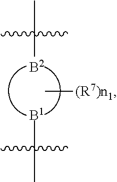
the ring where B¹, B² are located is a 6- to 9-membered nitrogen-containing heterocycloalkyl or a 6- to 9-membered nitrogen-containing heterocycloalkenyl, wherein the cycloalkyl or cycloalkenyl is a monocyclic ring, spirocyclic ring or bridged cyclic ring;
R¹ is H;
R² is H;
the ‘ [symbol] ’ in ring E represents ‘ [symbol] ’ is a bond or absent, when ‘ [symbol] ’ is a bond, R¹¹ is absent, ring E is 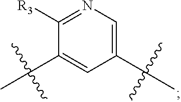
when ‘ [symbol] ’ is absent, ring E is 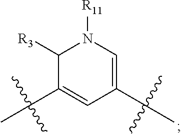
when R¹¹ exists, R¹¹ is selected from hydrogen or methyl;
R⁸ is selected from hydrogen, Cl or —CH₃; preferably R⁸ is hydrogen;
R²⁹ is selected from hydrogen, OH, —CN, F or —CF; preferably R²⁹ is hydrogen;
R³ is selected from hydrogen, halogen, cyano, C1˜C6 alkyl, —OR^{g}, —C(O)OR^{g}, —SR^{g} or —NR^{h}Rⁱ, when the ‘ [symbol] ’ in ring E is absent, R³ can also be a carbonyl group that together formed by the connected carbon atom, wherein the alkyl is optionally substituted by one or more halogen or —OH;
each occurrence of R^{g}, R^{h}, Rⁱ is independently selected from H, methyl, cyclopropyl, cyclopentyl or ethyl;
preferably methyl;
R⁴ is selected from C1˜C3 alkyl, C3˜C6 cycloalkyl, phenyl, naphthyl, 5- to 6-membered nitrogen-containing heteroaryl or 5- to 6-membered sulphur-containing heteroaryl, wherein the alkyl, cycloalkyl, aryl, nitrogen-containing heteroaryl, sulphur-containing heteroaryl are optionally substituted by 1˜3 R⁶;
each occurrence of R²⁶ is independently selected from halogen, cyano, C1˜C3 alkyl, halogenated C1˜C3 alkyl, —O(C1˜C3 alkyl) or —C(O)OH;
R⁵, R⁶ are independently selected from hydrogen, halogen, C1˜C3 alkyl, 5- to 6-membered heteroaryl, —C(O)R¹⁶, —S(O)₂R¹⁶, —P(O)R¹⁶R¹⁷, —C(O)NR^{b}R^{c}, —C(O)OR^{a} or —OR^{a}, wherein the alkyl, heteroaryl are optionally substituted by one or more R¹⁸;
R¹⁶, R¹⁷ are independently selected from C1˜C3 alkyl;
each occurrence of R^{a}, R^{b}, R^{c} is independently selected from H and C1˜C3 alkyl;
each occurrence of R¹⁸ is independently selected from halogen, C1˜C3 alkyl, —OR^{a} or —NR^{b}R^{c};
each occurrence of R⁷ is independently selected from C1˜C3 alkyl, wherein the alkyl is optionally substituted by one or more substituents independently selected from halogen, cyano or C1˜C3 alkyl;
n₁ is select 0 or 1;
preferably, 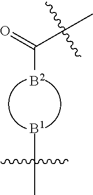
is selected from the following group:

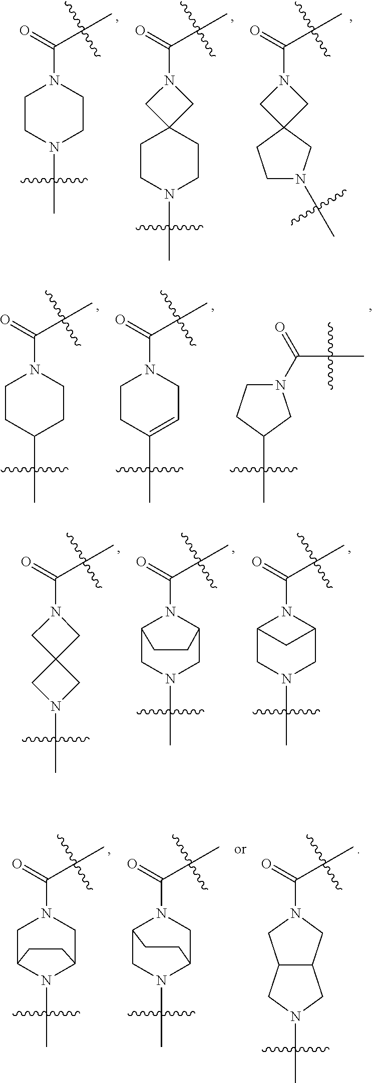

34. The compound according to claim 1, wherein the compound has a structure shown in formula VIII, or a tautomer, meso isomer, racemate, enantiomer, diastereomer or mixture thereof, or a pharmaceutically acceptable hydrate, solvate or salt thereof: 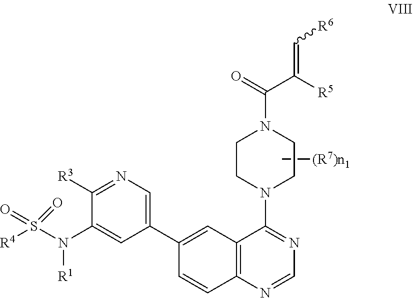
wherein,
R¹ is H;
R³ is selected from hydrogen, halogen, cyano, C1˜C6 alkyl, —OR^{g}, —C(O)OR^{g}, —SR^{g} or —NR^{h}Rⁱ, wherein the alkyl is optionally substituted by one or more halogen or —OH;
each occurrence of R^{g}, R^{h}, Rⁱ is independently selected from H, methyl, cyclopropyl, cyclopentyl or ethyl;
preferably methyl;
R⁴ is selected from C1˜C3 alkyl, cyclohexyl, phenyl, pyridyl, thienyl, naphthyl, pyrrolyl or thiazolyl, wherein alkyl, cyclohexyl, phenyl, pyridyl, thienyl, naphthyl, pyrrolyl, thiazolyl are optionally substituted by 1˜2 R²⁶;
each occurrence of R²⁶ is independently selected from F, Cl, Br or cyano, unsubstituted C1˜C3 alkyl or C1˜C3 alkyl substituted by 1 to 3 halogen, —OR^{j}, —C(O)OR^{j};
R^{j} is selected from H and C1˜C3 alkyl;
R⁵, R⁶ are independently selected from hydrogen, halogen, C1˜C3 alkyl, 5- to 6-membered heteroaryl, —C(O)R¹⁶, —S(O)₂R¹⁶, —P(O)R¹⁶R¹⁷, —C(O)NR^{b}R^{c}, —C(O)OR^{a} or —OR^{a}, wherein the alkyl, heteroaryl are optionally substituted by one or more R¹⁸;
R¹⁶, R¹⁷ is independently selected from C1˜C3 alkyl;
each occurrence of R¹⁸ is independently selected from halogen, C1˜C3 alkyl, —OR^{a} or —NR^{b}R^{c};
each occurrence of R^{a}, R^{b}, R^{c} is independently selected from H and C1˜C3 alkyl;
each occurrence of R⁷ is independently selected from C1˜C3 alkyl, wherein the alkyl is optionally substituted by one or more substituents independently selected from halogen, cyano or C1˜C3 alkyl;
n₁ is selected from 0 or 1.

35. The compound according to claim 34 or a tautomer, meso isomer, racemate, enantiomer, diastereomer or mixture thereof, or a pharmaceutically acceptable hydrate, solvate or salt thereof:
wherein,
R¹ is H;
R³ is selected from hydrogen, Cl, —CF₃, —OCH₃, —OCH₂CH₃, —OCF₃, —OCHF₂, —N(CH)₂, —CH₃, —CH₂OH, —OCH₂CF₃, —OH, —NHCH—, —SCH—, —OCD₃, —CN or —C(O)OCH₃;
R⁴ is selected from methyl, cyclohexyl, 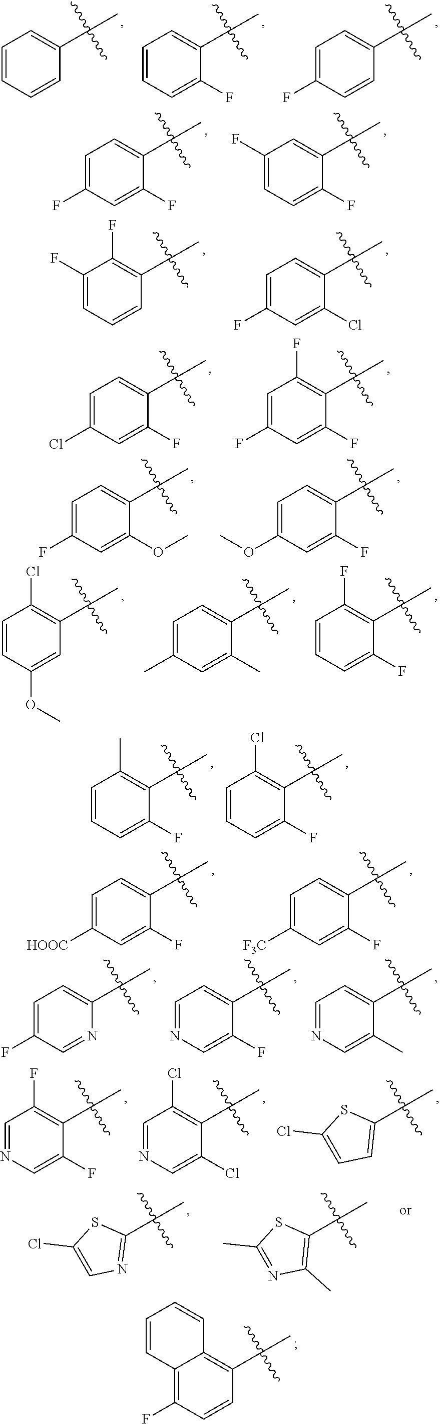
R⁵ is selected from H, F, CN, —CH(CH₃)₂, —CH(OH)CH₃, —CH₂OCH₃, —OCH₃ or —C(O)CH₃;
R⁶ is selected from H, —C(CH₃)₃, —C(O)CH₃, —C(O)CH₂CH₂CH₃, —C(O)NHCH₃), —CH₂N(CH₃)₂, —CH₂F, — 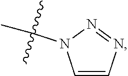 [SMILES: *n1ccnn1]
CH₂OCH₃, CF₃, 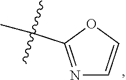 [SMILES: *c1ncco1]
—C(O)OCH₃, —P(O)(CH₃), —S(O)₂CH₃, —C(O)CF₃ or —C(O)CH₂CH₃;
R⁷ is selected from hydrogen, methyl or —CH₂CN;
n₁ is selected from 0 or 1.

36. The following compounds, or a tautomer, meso isomer, racemate, enantiomer, diastereomer or mixture thereof, or a pharmaceutically acceptable hydrate, solvate or salt thereof:

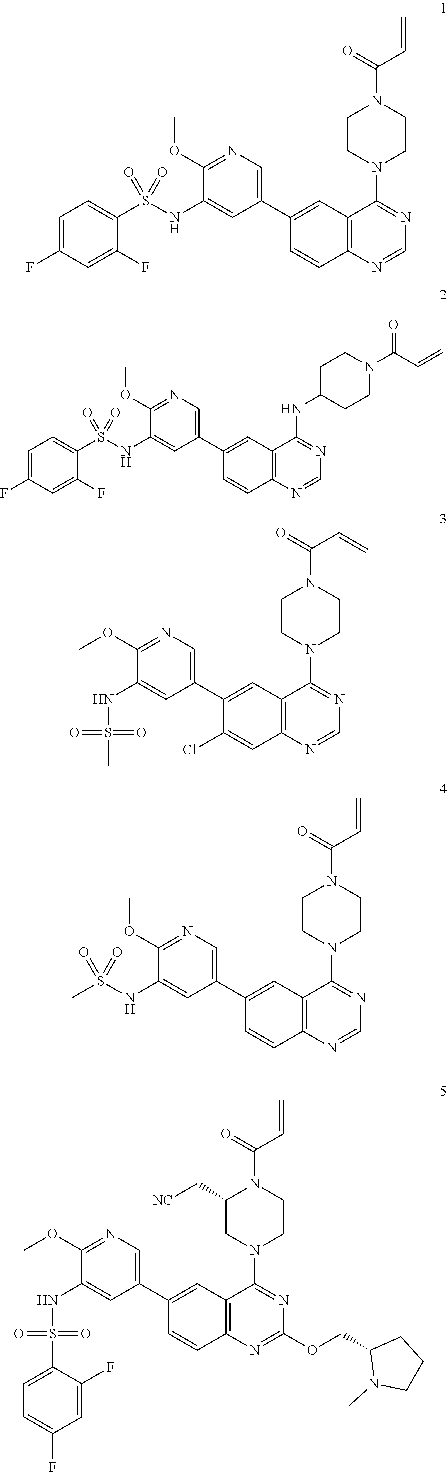

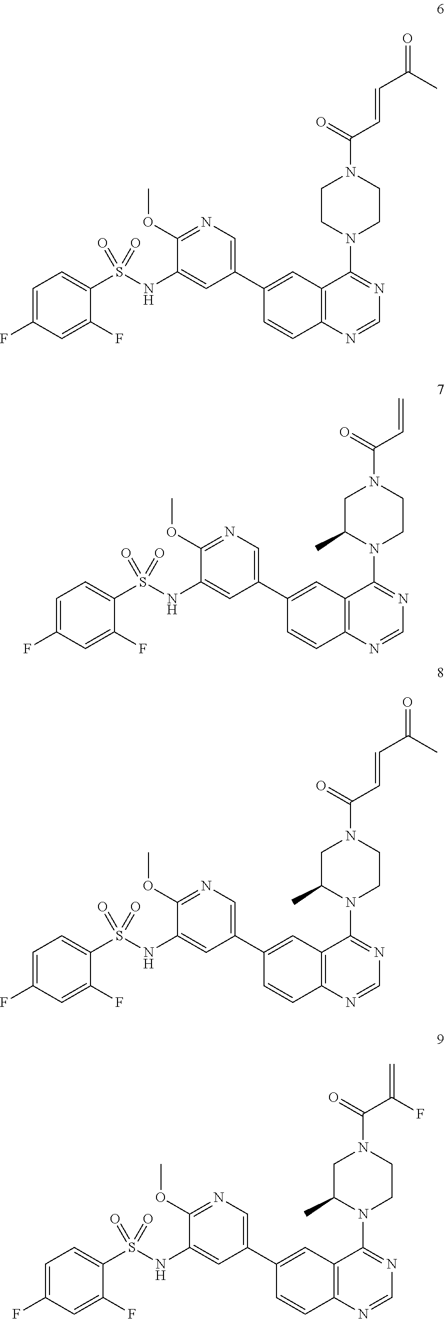 [SMILES: COc1ncc(-c2ccc3ncnc(N4CCN(C(=O)/C=C/C(C)=O)CC4)c3c2)cc1NS(=O)(=O)c1ccc(F)cc1F ; C=CC(=O)N1CCN(c2ncnc3ccc(-c4cnc(OC)c(NS(=O)(=O)c5ccc(F)cc5F)c4)cc23)[C@@H](C)C1 ; COc1ncc(-c2ccc3ncnc(N4CCN(C(=O)/C=C/C(C)=O)C[C@@H]4C)c3c2)cc1NS(=O)(=O)c1ccc(F)cc1F ; C=C(F)C(=O)N1CCN(c2ncnc3ccc(-c4cnc(OC)c(NS(=O)(=O)c5ccc(F)cc5F)c4)cc23)[C@@H](C)C1]

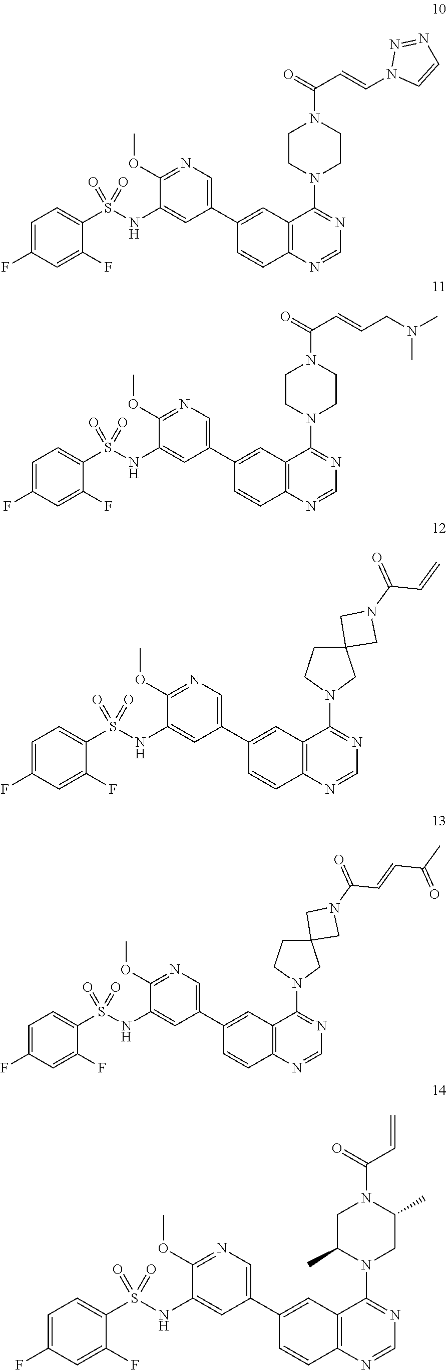 [SMILES: COc1ncc(-c2ccc3ncnc(N4CCN(C(=O)/C=C/n5ccnn5)CC4)c3c2)cc1NS(=O)(=O)c1ccc(F)cc1F ; COc1ncc(-c2ccc3ncnc(N4CCN(C(=O)/C=C/CN(C)C)CC4)c3c2)cc1NS(=O)(=O)c1ccc(F)cc1F ; C=CC(=O)N1CC2(CCN(c3ncnc4ccc(-c5cnc(OC)c(NS(=O)(=O)c6ccc(F)cc6F)c5)cc34)C2)C1 ; COc1ncc(-c2ccc3ncnc(N4CCC5(CN(C(=O)/C=C/C(C)=O)C5)C4)c3c2)cc1NS(=O)(=O)c1ccc(F)cc1F ; C=CC(=O)N1C[C@H](C)N(c2ncnc3ccc(-c4cnc(OC)c(NS(=O)(=O)c5ccc(F)cc5F)c4)cc23)C[C@H]1C]

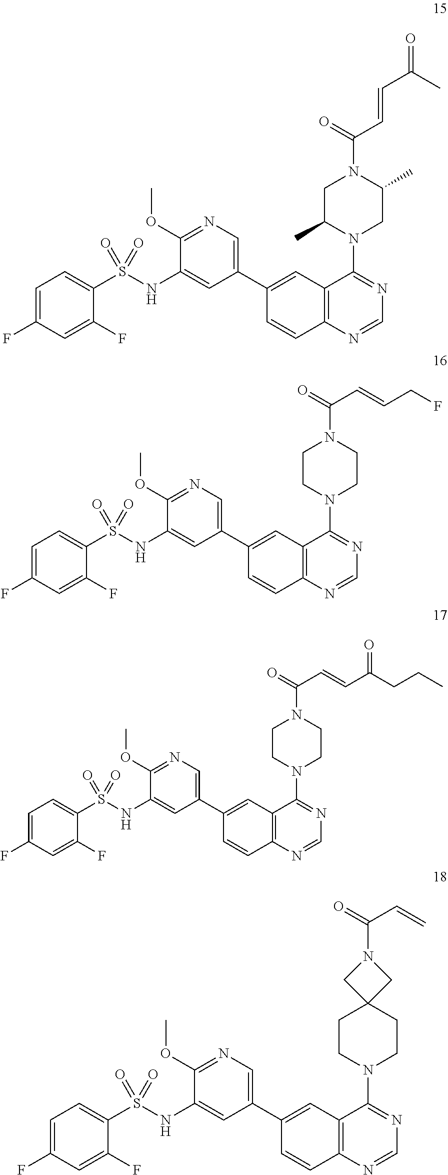 [SMILES: COc1ncc(-c2ccc3ncnc(N4C[C@@H](C)N(C(=O)/C=C/C(C)=O)C[C@@H]4C)c3c2)cc1NS(=O)(=O)c1ccc(F)cc1F ; COc1ncc(-c2ccc3ncnc(N4CCN(C(=O)/C=C/CF)CC4)c3c2)cc1NS(=O)(=O)c1ccc(F)cc1F ; CCCC(=O)/C=C/C(=O)N1CCN(c2ncnc3ccc(-c4cnc(OC)c(NS(=O)(=O)c5ccc(F)cc5F)c4)cc23)CC1 ; C=CC(=O)N1CC2(CCN(c3ncnc4ccc(-c5cnc(OC)c(NS(=O)(=O)c6ccc(F)cc6F)c5)cc34)CC2)C1]

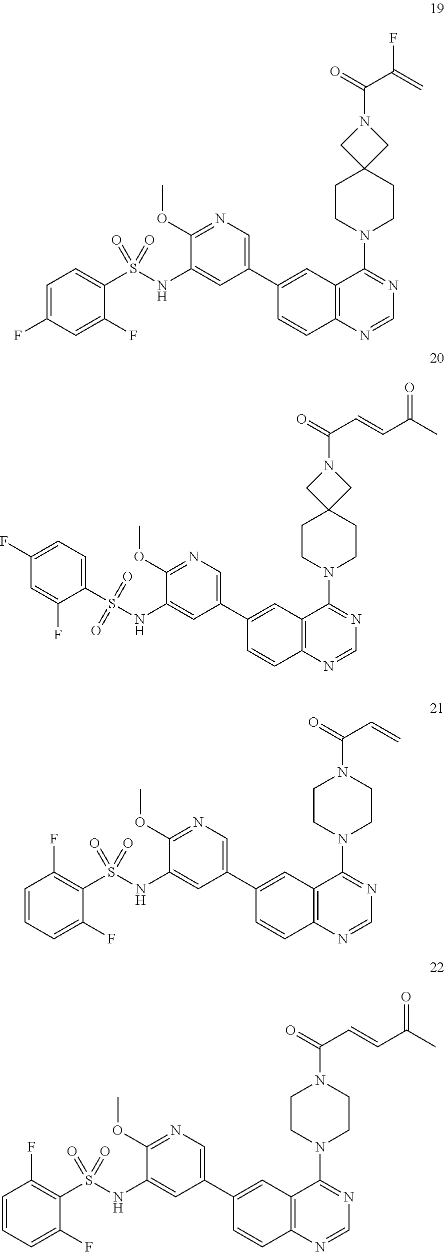 [SMILES: C=C(F)C(=O)N1CC2(CCN(c3ncnc4ccc(-c5cnc(OC)c(NS(=O)(=O)c6ccc(F)cc6F)c5)cc34)CC2)C1 ; COc1ncc(-c2ccc3ncnc(N4CCC5(CC4)CN(C(=O)/C=C/C(C)=O)C5)c3c2)cc1NS(=O)(=O)c1ccc(F)cc1F ; C=CC(=O)N1CCN(c2ncnc3ccc(-c4cnc(OC)c(NS(=O)(=O)c5c(F)cccc5F)c4)cc23)CC1 ; COc1ncc(-c2ccc3ncnc(N4CCN(C(=O)/C=C/C(C)=O)CC4)c3c2)cc1NS(=O)(=O)c1c(F)cccc1F]

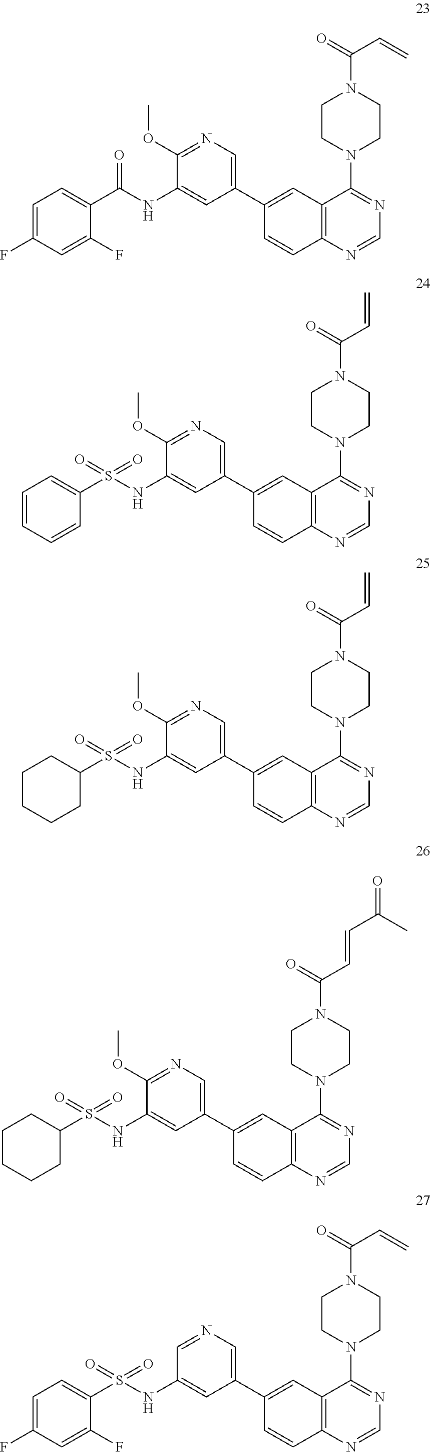

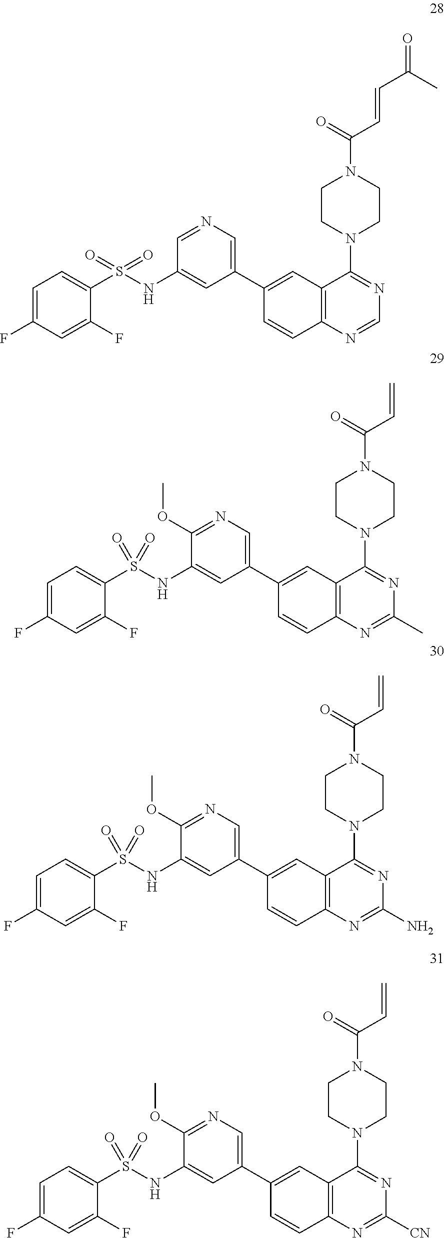

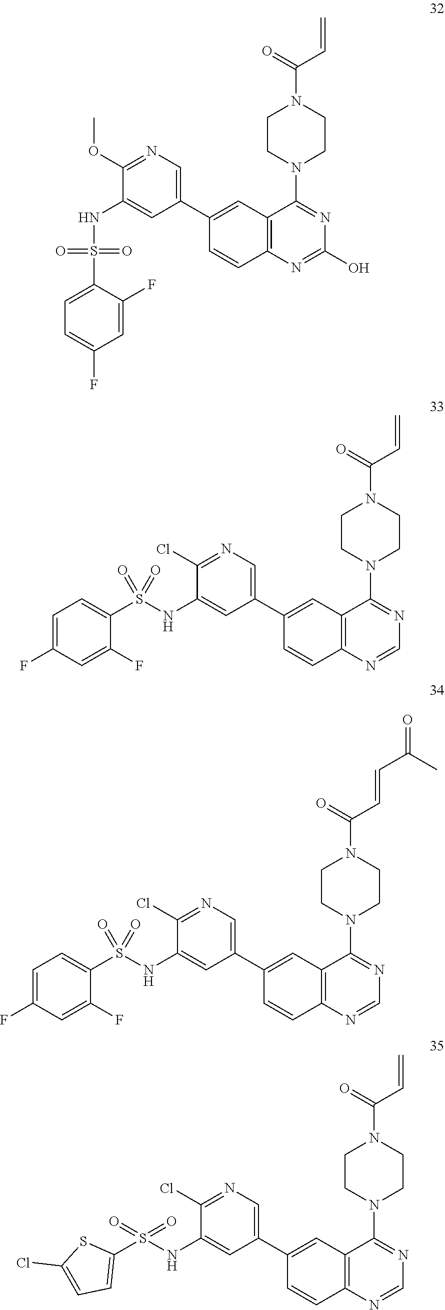 [SMILES: C=CC(=O)N1CCN(c2nc(O)nc3ccc(-c4cnc(OC)c(NS(=O)(=O)c5ccc(F)cc5F)c4)cc23)CC1 ; C=CC(=O)N1CCN(c2ncnc3ccc(-c4cnc(Cl)c(NS(=O)(=O)c5ccc(F)cc5F)c4)cc23)CC1 ; CC(=O)/C=C/C(=O)N1CCN(c2ncnc3ccc(-c4cnc(Cl)c(NS(=O)(=O)c5ccc(F)cc5F)c4)cc23)CC1 ; C=CC(=O)N1CCN(c2ncnc3ccc(-c4cnc(Cl)c(NS(=O)(=O)c5ccc(Cl)s5)c4)cc23)CC1]

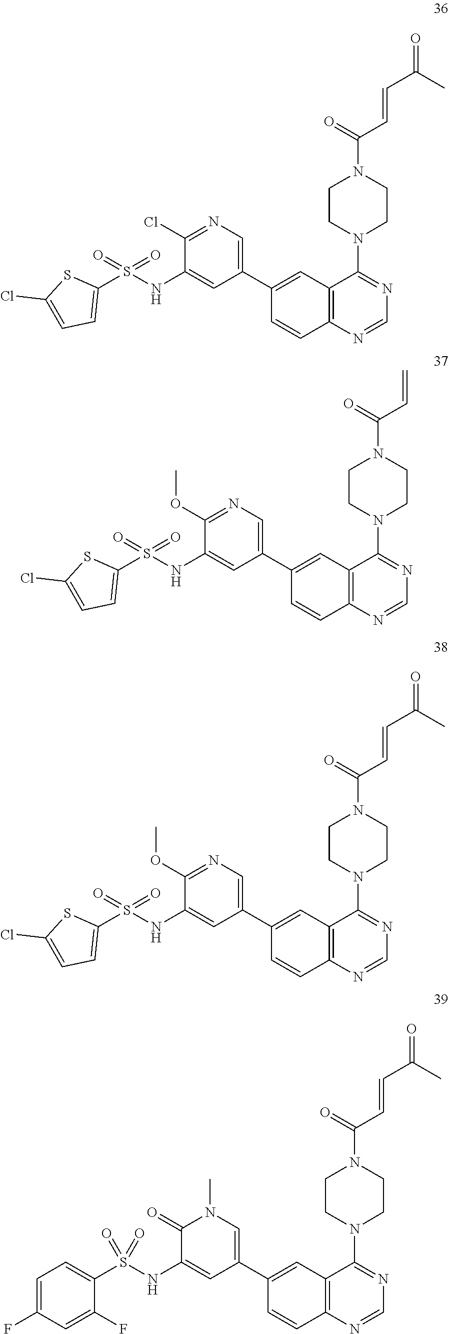 [SMILES: CC(=O)/C=C/C(=O)N1CCN(c2ncnc3ccc(-c4cnc(Cl)c(NS(=O)(=O)c5ccc(Cl)s5)c4)cc23)CC1 ; C=CC(=O)N1CCN(c2ncnc3ccc(-c4cnc(OC)c(NS(=O)(=O)c5ccc(Cl)s5)c4)cc23)CC1 ; COc1ncc(-c2ccc3ncnc(N4CCN(C(=O)/C=C/C(C)=O)CC4)c3c2)cc1NS(=O)(=O)c1ccc(Cl)s1 ; CC(=O)/C=C/C(=O)N1CCN(c2ncnc3ccc(-c4cc(NS(=O)(=O)c5ccc(F)cc5F)c(=O)n(C)c4)cc23)CC1]

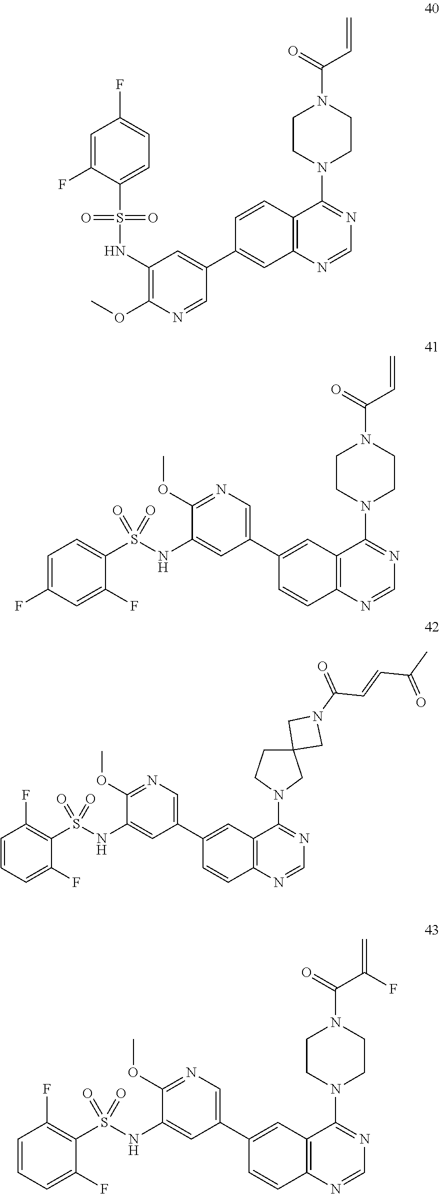 [SMILES: C=CC(=O)N1CCN(c2ncnc3cc(-c4cnc(OC)c(NS(=O)(=O)c5ccc(F)cc5F)c4)ccc23)CC1 ; C=CC(=O)N1CCN(c2ncnc3ccc(-c4cnc(OC)c(NS(=O)(=O)c5ccc(F)cc5F)c4)cc23)CC1 ; COc1ncc(-c2ccc3ncnc(N4CCC5(CN(C(=O)/C=C/C(C)=O)C5)C4)c3c2)cc1NS(=O)(=O)c1c(F)cccc1F ; C=C(F)C(=O)N1CCN(c2ncnc3ccc(-c4cnc(OC)c(NS(=O)(=O)c5c(F)cccc5F)c4)cc23)CC1]

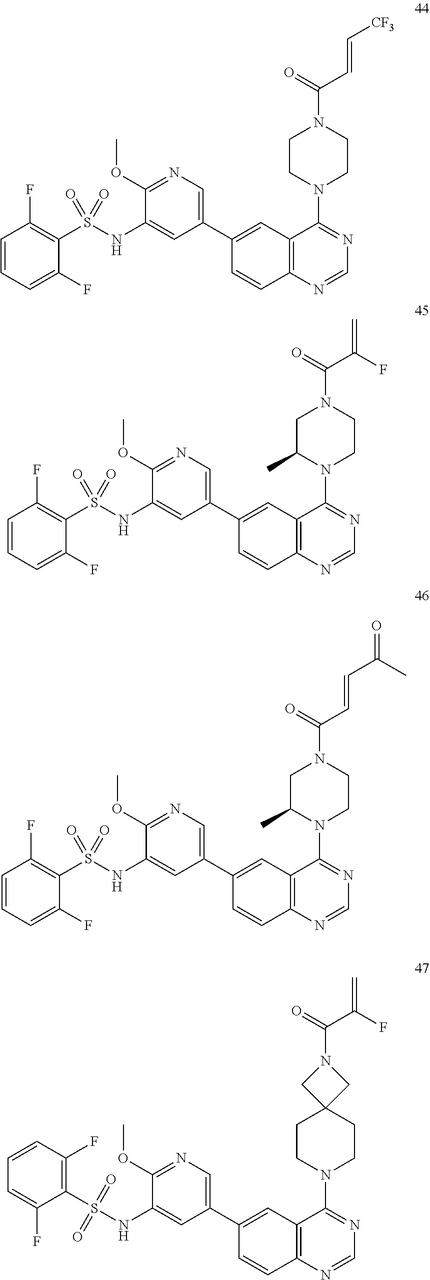 [SMILES: COc1ncc(-c2ccc3ncnc(N4CCN(C(=O)/C=C/C(F)(F)F)CC4)c3c2)cc1NS(=O)(=O)c1c(F)cccc1F ; C=C(F)C(=O)N1CCN(c2ncnc3ccc(-c4cnc(OC)c(NS(=O)(=O)c5c(F)cccc5F)c4)cc23)[C@@H](C)C1 ; COc1ncc(-c2ccc3ncnc(N4CCN(C(=O)/C=C/C(C)=O)C[C@@H]4C)c3c2)cc1NS(=O)(=O)c1c(F)cccc1F ; C=C(F)C(=O)N1CC2(CCN(c3ncnc4ccc(-c5cnc(OC)c(NS(=O)(=O)c6c(F)cccc6F)c5)cc34)CC2)C1]

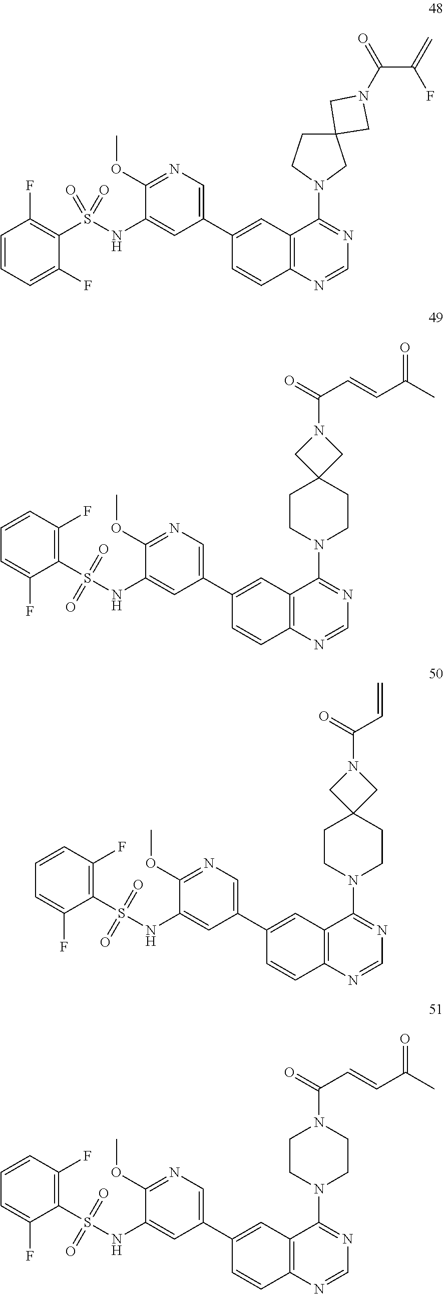 [SMILES: C=C(F)C(=O)N1CC2(CCN(c3ncnc4ccc(-c5cnc(OC)c(NS(=O)(=O)c6c(F)cccc6F)c5)cc34)C2)C1 ; COc1ncc(-c2ccc3ncnc(N4CCC5(CC4)CN(C(=O)/C=C/C(C)=O)C5)c3c2)cc1NS(=O)(=O)c1c(F)cccc1F ; C=CC(=O)N1CC2(CCN(c3ncnc4ccc(-c5cnc(OC)c(NS(=O)(=O)c6c(F)cccc6F)c5)cc34)CC2)C1 ; COc1ncc(-c2ccc3ncnc(N4CCN(C(=O)/C=C/C(C)=O)CC4)c3c2)cc1NS(=O)(=O)c1c(F)cccc1F]

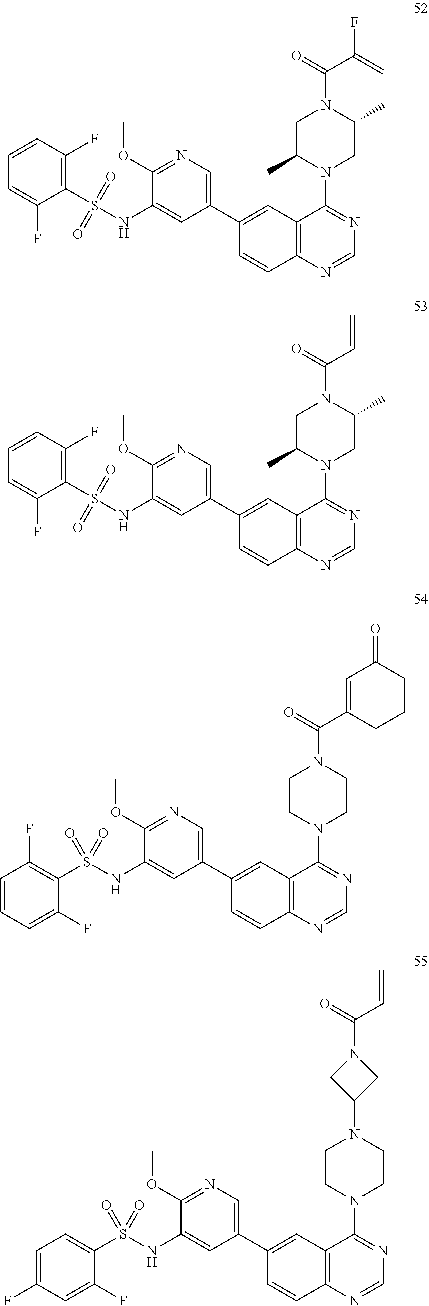 [SMILES: C=C(F)C(=O)N1C[C@H](C)N(c2ncnc3ccc(-c4cnc(OC)c(NS(=O)(=O)c5c(F)cccc5F)c4)cc23)C[C@H]1C ; C=CC(=O)N1C[C@H](C)N(c2ncnc3ccc(-c4cnc(OC)c(NS(=O)(=O)c5c(F)cccc5F)c4)cc23)C[C@H]1C ; COc1ncc(-c2ccc3ncnc(N4CCN(C(=O)C5=CC(=O)CCC5)CC4)c3c2)cc1NS(=O)(=O)c1c(F)cccc1F ; C=CC(=O)N1CC(N2CCN(c3ncnc4ccc(-c5cnc(OC)c(NS(=O)(=O)c6ccc(F)cc6F)c5)cc34)CC2)C1]

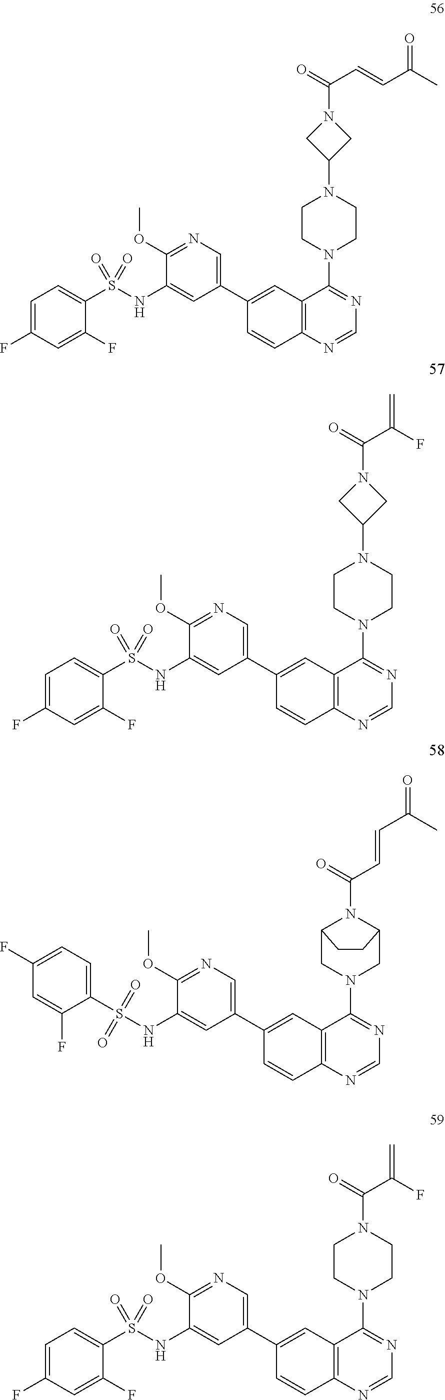 [SMILES: COc1ncc(-c2ccc3ncnc(N4CCN(C5CN(C(=O)/C=C/C(C)=O)C5)CC4)c3c2)cc1NS(=O)(=O)c1ccc(F)cc1F ; C=C(F)C(=O)N1CC(N2CCN(c3ncnc4ccc(-c5cnc(OC)c(NS(=O)(=O)c6ccc(F)cc6F)c5)cc34)CC2)C1 ; COc1ncc(-c2ccc3ncnc(N4CC5CCC(C4)N5C(=O)/C=C/C(C)=O)c3c2)cc1NS(=O)(=O)c1ccc(F)cc1F ; C=C(F)C(=O)N1CCN(c2ncnc3ccc(-c4cnc(OC)c(NS(=O)(=O)c5ccc(F)cc5F)c4)cc23)CC1]

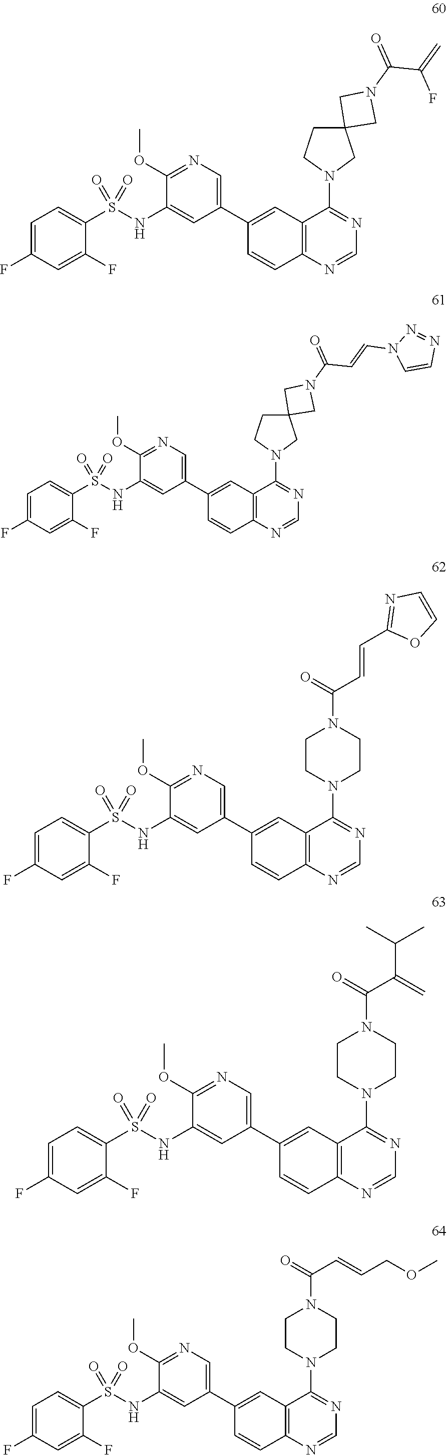 [SMILES: C=C(F)C(=O)N1CC2(CCN(c3ncnc4ccc(-c5cnc(OC)c(NS(=O)(=O)c6ccc(F)cc6F)c5)cc34)C2)C1 ; COc1ncc(-c2ccc3ncnc(N4CCC5(CN(C(=O)/C=C/n6ccnn6)C5)C4)c3c2)cc1NS(=O)(=O)c1ccc(F)cc1F ; COc1ncc(-c2ccc3ncnc(N4CCN(C(=O)/C=C/c5ncco5)CC4)c3c2)cc1NS(=O)(=O)c1ccc(F)cc1F ; C=C(C(=O)N1CCN(c2ncnc3ccc(-c4cnc(OC)c(NS(=O)(=O)c5ccc(F)cc5F)c4)cc23)CC1)C(C)C ; COC/C=C/C(=O)N1CCN(c2ncnc3ccc(-c4cnc(OC)c(NS(=O)(=O)c5ccc(F)cc5F)c4)cc23)CC1]

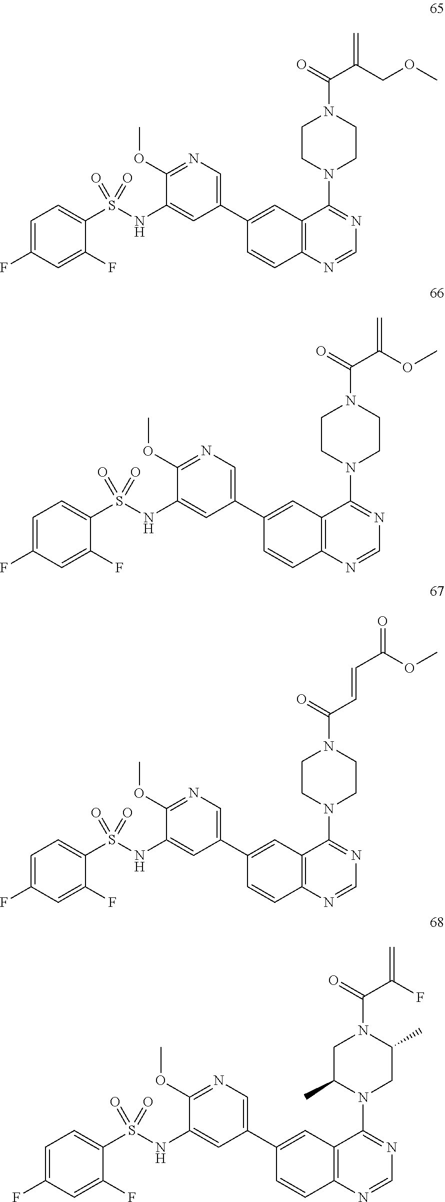 [SMILES: C=C(COC)C(=O)N1CCN(c2ncnc3ccc(-c4cnc(OC)c(NS(=O)(=O)c5ccc(F)cc5F)c4)cc23)CC1 ; C=C(OC)C(=O)N1CCN(c2ncnc3ccc(-c4cnc(OC)c(NS(=O)(=O)c5ccc(F)cc5F)c4)cc23)CC1 ; COC(=O)/C=C/C(=O)N1CCN(c2ncnc3ccc(-c4cnc(OC)c(NS(=O)(=O)c5ccc(F)cc5F)c4)cc23)CC1 ; C=C(F)C(=O)N1C[C@H](C)N(c2ncnc3ccc(-c4cnc(OC)c(NS(=O)(=O)c5ccc(F)cc5F)c4)cc23)C[C@H]1C]

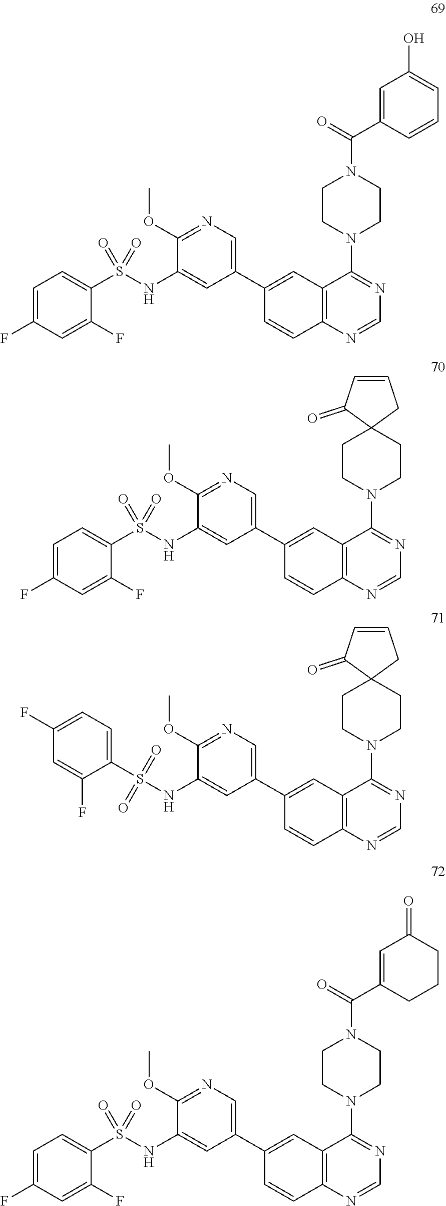

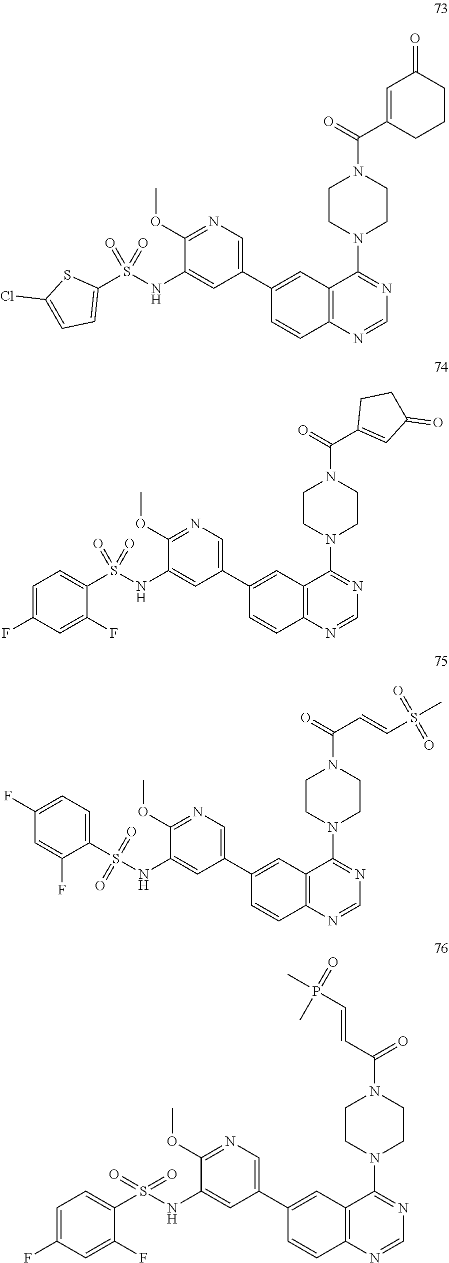

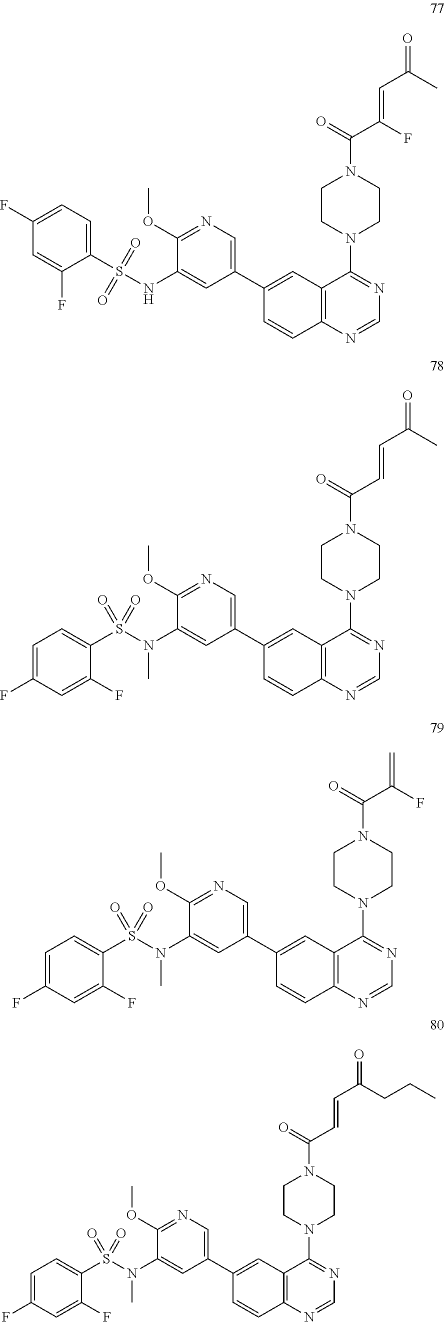 [SMILES: COc1ncc(-c2ccc3ncnc(N4CCN(C(=O)/C(F)=C/C(C)=O)CC4)c3c2)cc1NS(=O)(=O)c1ccc(F)cc1F ; COc1ncc(-c2ccc3ncnc(N4CCN(C(=O)/C=C/C(C)=O)CC4)c3c2)cc1N(C)S(=O)(=O)c1ccc(F)cc1F ; C=C(F)C(=O)N1CCN(c2ncnc3ccc(-c4cnc(OC)c(N(C)S(=O)(=O)c5ccc(F)cc5F)c4)cc23)CC1 ; CCCC(=O)/C=C/C(=O)N1CCN(c2ncnc3ccc(-c4cnc(OC)c(N(C)S(=O)(=O)c5ccc(F)cc5F)c4)cc23)CC1]

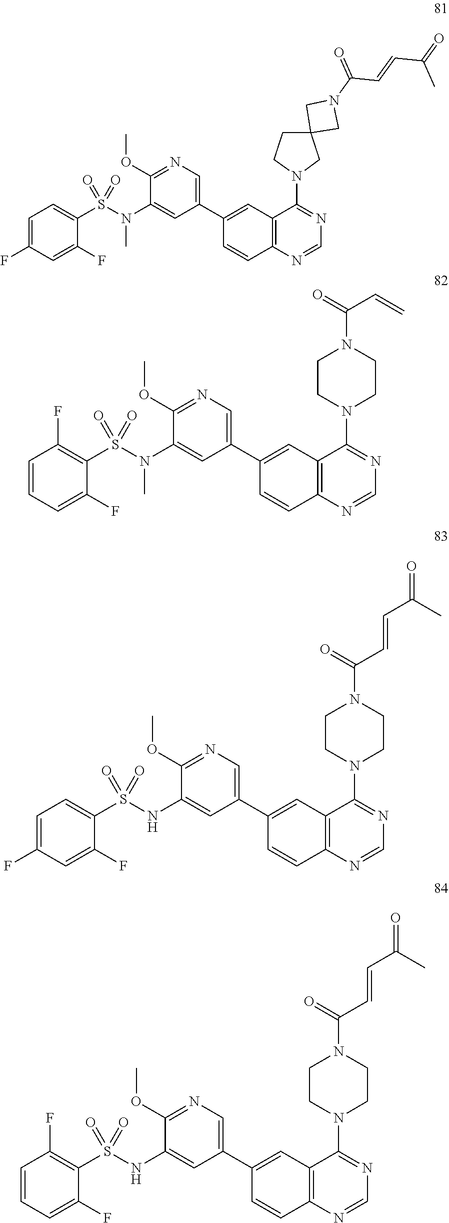

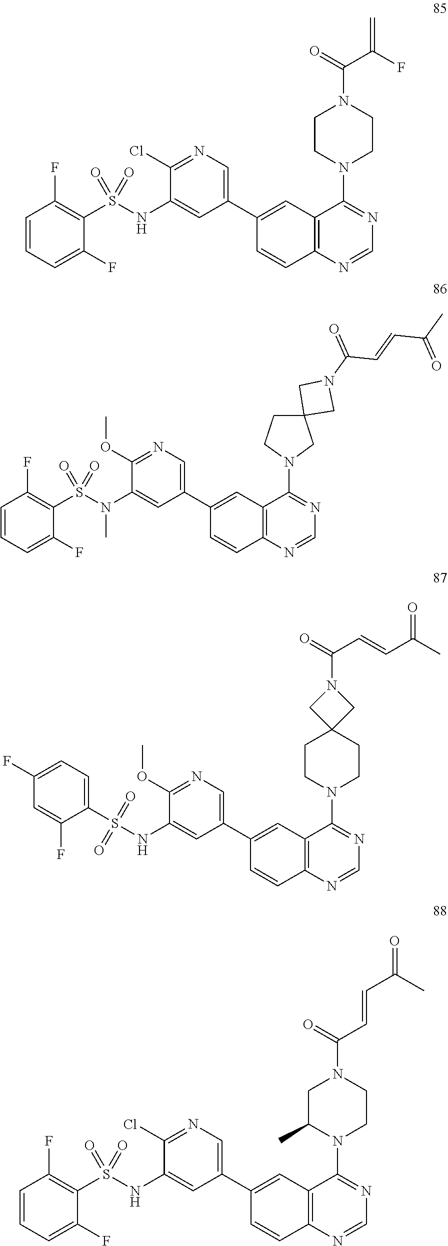 [SMILES: C=C(F)C(=O)N1CCN(c2ncnc3ccc(-c4cnc(Cl)c(NS(=O)(=O)c5c(F)cccc5F)c4)cc23)CC1 ; COc1ncc(-c2ccc3ncnc(N4CCC5(CN(C(=O)/C=C/C(C)=O)C5)C4)c3c2)cc1N(C)S(=O)(=O)c1c(F)cccc1F ; COc1ncc(-c2ccc3ncnc(N4CCC5(CC4)CN(C(=O)/C=C/C(C)=O)C5)c3c2)cc1NS(=O)(=O)c1ccc(F)cc1F ; CC(=O)/C=C/C(=O)N1CCN(c2ncnc3ccc(-c4cnc(Cl)c(NS(=O)(=O)c5c(F)cccc5F)c4)cc23)[C@@H](C)C1]

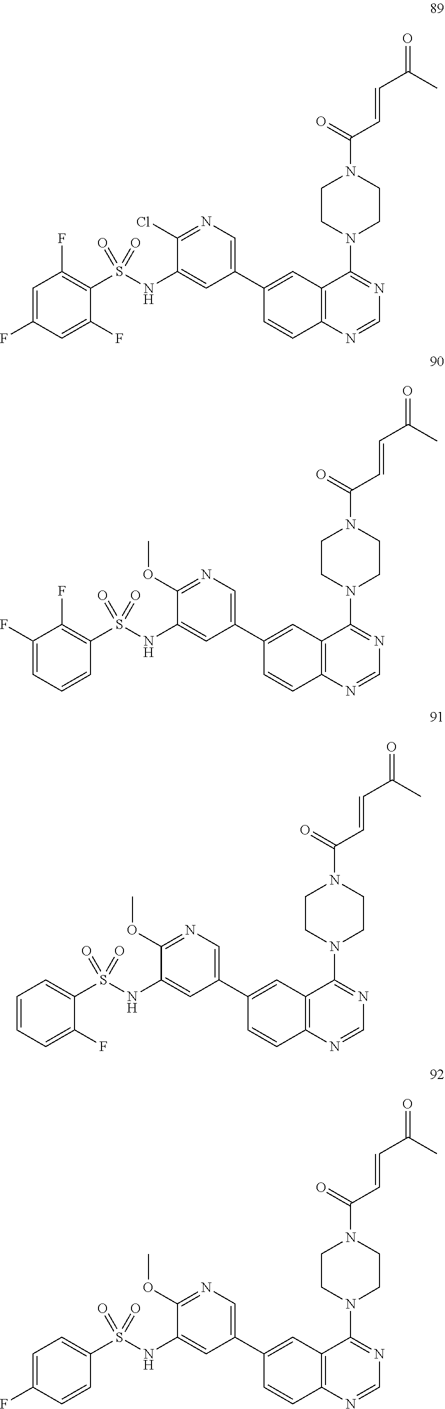 [SMILES: CC(=O)/C=C/C(=O)N1CCN(c2ncnc3ccc(-c4cnc(Cl)c(NS(=O)(=O)c5c(F)cc(F)cc5F)c4)cc23)CC1 ; COc1ncc(-c2ccc3ncnc(N4CCN(C(=O)/C=C/C(C)=O)CC4)c3c2)cc1NS(=O)(=O)c1cccc(F)c1F ; COc1ncc(-c2ccc3ncnc(N4CCN(C(=O)/C=C/C(C)=O)CC4)c3c2)cc1NS(=O)(=O)c1ccccc1F ; COc1ncc(-c2ccc3ncnc(N4CCN(C(=O)/C=C/C(C)=O)CC4)c3c2)cc1NS(=O)(=O)c1ccc(F)cc1]

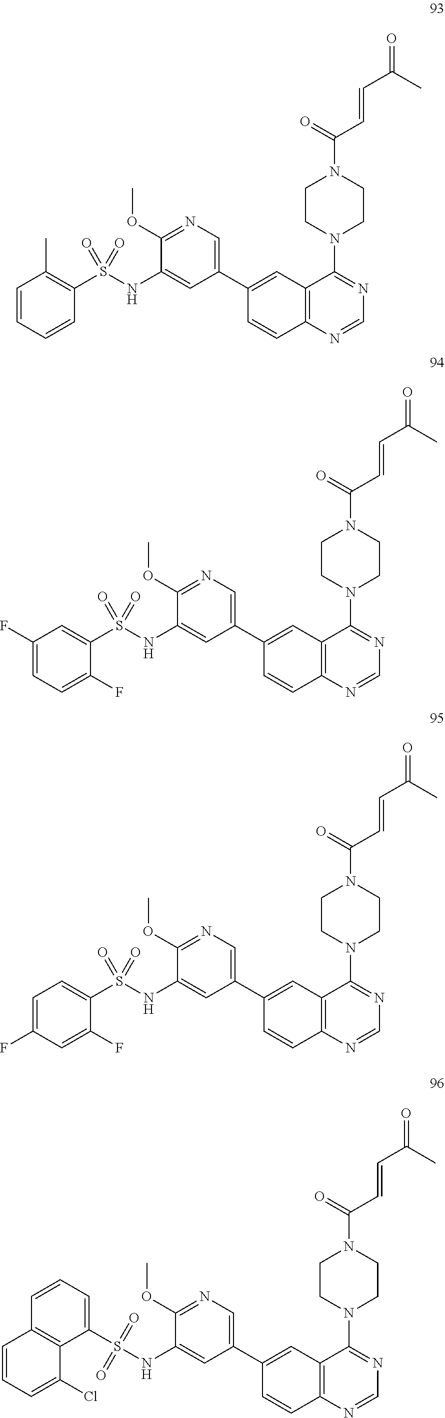 [SMILES: COc1ncc(-c2ccc3ncnc(N4CCN(C(=O)/C=C/C(C)=O)CC4)c3c2)cc1NS(=O)(=O)c1ccccc1C ; COc1ncc(-c2ccc3ncnc(N4CCN(C(=O)/C=C/C(C)=O)CC4)c3c2)cc1NS(=O)(=O)c1cc(F)ccc1F ; COc1ncc(-c2ccc3ncnc(N4CCN(C(=O)/C=C/C(C)=O)CC4)c3c2)cc1NS(=O)(=O)c1ccc(F)cc1F ; COc1ncc(-c2ccc3ncnc(N4CCN(C(=O)/C=C/C(C)=O)CC4)c3c2)cc1NS(=O)(=O)c1cccc2cccc(Cl)c12]

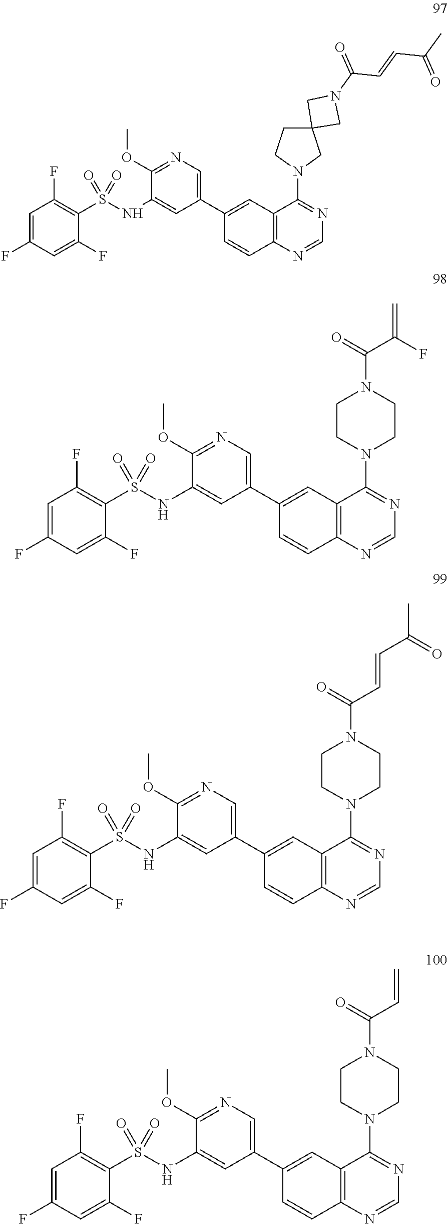 [SMILES: COc1ncc(-c2ccc3ncnc(N4CCC5(CN(C(=O)/C=C/C(C)=O)C5)C4)c3c2)cc1NS(=O)(=O)c1c(F)cc(F)cc1F ; C=C(F)C(=O)N1CCN(c2ncnc3ccc(-c4cnc(OC)c(NS(=O)(=O)c5c(F)cc(F)cc5F)c4)cc23)CC1 ; COc1ncc(-c2ccc3ncnc(N4CCN(C(=O)/C=C/C(C)=O)CC4)c3c2)cc1NS(=O)(=O)c1c(F)cc(F)cc1F ; C=CC(=O)N1CCN(c2ncnc3ccc(-c4cnc(OC)c(NS(=O)(=O)c5c(F)cc(F)cc5F)c4)cc23)CC1]

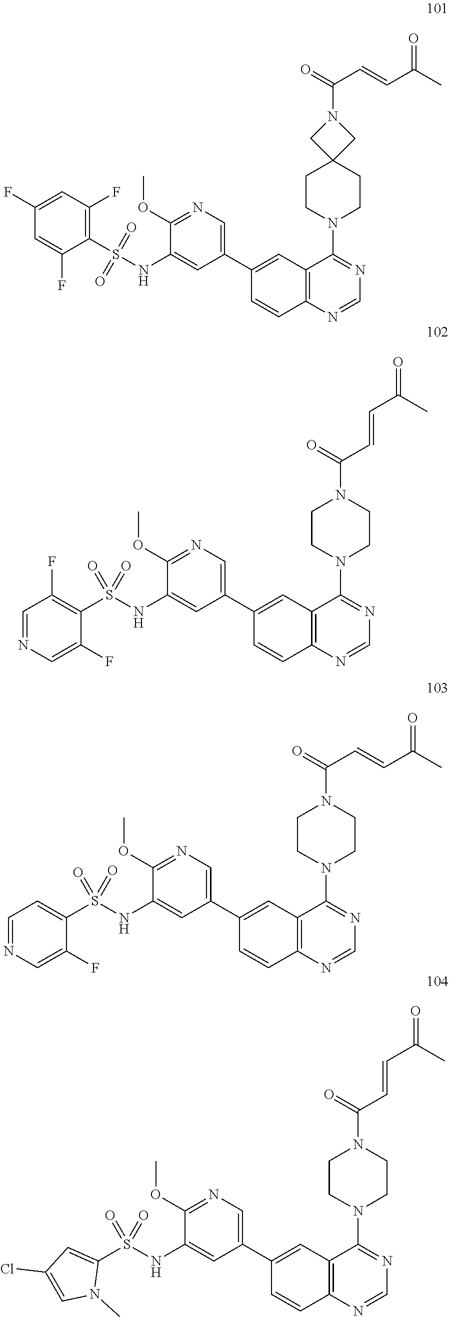 [SMILES: COc1ncc(-c2ccc3ncnc(N4CCC5(CC4)CN(C(=O)/C=C/C(C)=O)C5)c3c2)cc1NS(=O)(=O)c1c(F)cc(F)cc1F ; COc1ncc(-c2ccc3ncnc(N4CCN(C(=O)/C=C/C(C)=O)CC4)c3c2)cc1NS(=O)(=O)c1c(F)cncc1F ; COc1ncc(-c2ccc3ncnc(N4CCN(C(=O)/C=C/C(C)=O)CC4)c3c2)cc1NS(=O)(=O)c1ccncc1F ; COc1ncc(-c2ccc3ncnc(N4CCN(C(=O)/C=C/C(C)=O)CC4)c3c2)cc1NS(=O)(=O)c1cc(Cl)cn1C]

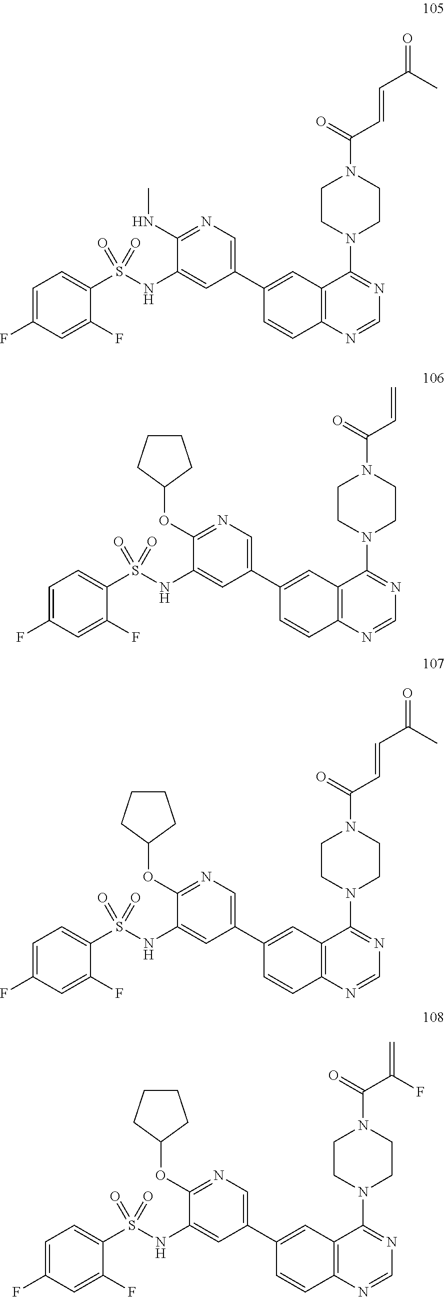 [SMILES: CNc1ncc(-c2ccc3ncnc(N4CCN(C(=O)/C=C/C(C)=O)CC4)c3c2)cc1NS(=O)(=O)c1ccc(F)cc1F ; C=CC(=O)N1CCN(c2ncnc3ccc(-c4cnc(OC5CCCC5)c(NS(=O)(=O)c5ccc(F)cc5F)c4)cc23)CC1 ; CC(=O)/C=C/C(=O)N1CCN(c2ncnc3ccc(-c4cnc(OC5CCCC5)c(NS(=O)(=O)c5ccc(F)cc5F)c4)cc23)CC1 ; C=C(F)C(=O)N1CCN(c2ncnc3ccc(-c4cnc(OC5CCCC5)c(NS(=O)(=O)c5ccc(F)cc5F)c4)cc23)CC1]

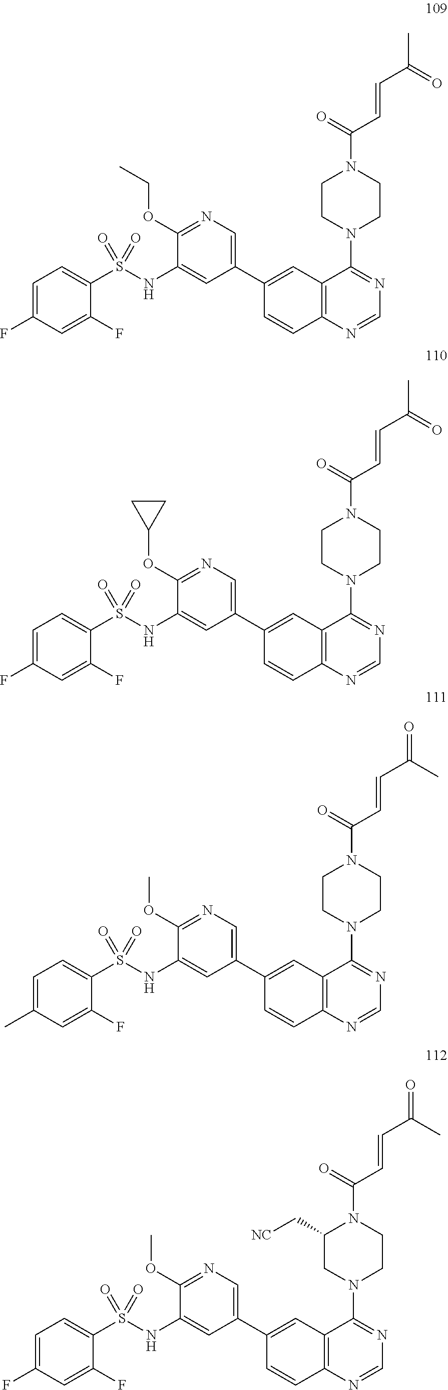 [SMILES: CCOc1ncc(-c2ccc3ncnc(N4CCN(C(=O)/C=C/C(C)=O)CC4)c3c2)cc1NS(=O)(=O)c1ccc(F)cc1F ; CC(=O)/C=C/C(=O)N1CCN(c2ncnc3ccc(-c4cnc(OC5CC5)c(NS(=O)(=O)c5ccc(F)cc5F)c4)cc23)CC1 ; COc1ncc(-c2ccc3ncnc(N4CCN(C(=O)/C=C/C(C)=O)CC4)c3c2)cc1NS(=O)(=O)c1ccc(C)cc1F ; COc1ncc(-c2ccc3ncnc(N4CCN(C(=O)/C=C/C(C)=O)[C@@H](CC#N)C4)c3c2)cc1NS(=O)(=O)c1ccc(F)cc1F]

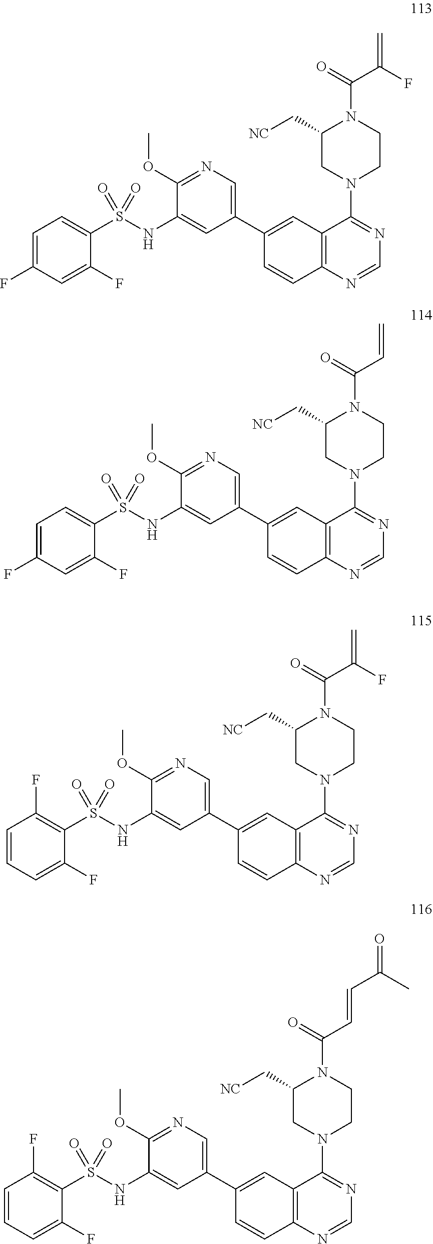 [SMILES: C=C(F)C(=O)N1CCN(c2ncnc3ccc(-c4cnc(OC)c(NS(=O)(=O)c5ccc(F)cc5F)c4)cc23)C[C@@H]1CC#N ; C=CC(=O)N1CCN(c2ncnc3ccc(-c4cnc(OC)c(NS(=O)(=O)c5ccc(F)cc5F)c4)cc23)C[C@@H]1CC#N ; C=C(F)C(=O)N1CCN(c2ncnc3ccc(-c4cnc(OC)c(NS(=O)(=O)c5c(F)cccc5F)c4)cc23)C[C@@H]1CC#N ; COc1ncc(-c2ccc3ncnc(N4CCN(C(=O)/C=C/C(C)=O)[C@@H](CC#N)C4)c3c2)cc1NS(=O)(=O)c1c(F)cccc1F]

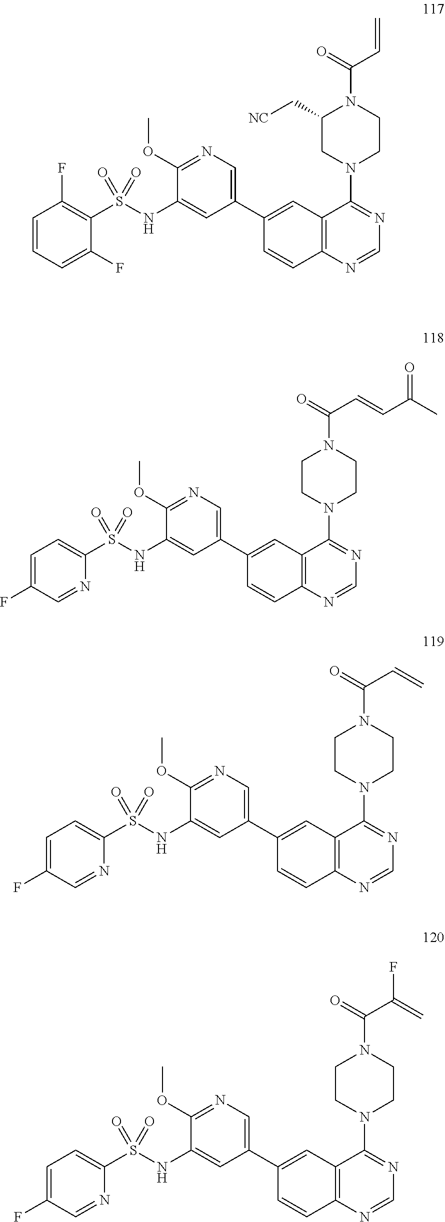 [SMILES: C=CC(=O)N1CCN(c2ncnc3ccc(-c4cnc(OC)c(NS(=O)(=O)c5c(F)cccc5F)c4)cc23)C[C@@H]1CC#N ; COc1ncc(-c2ccc3ncnc(N4CCN(C(=O)/C=C/C(C)=O)CC4)c3c2)cc1NS(=O)(=O)c1ccc(F)cn1 ; C=CC(=O)N1CCN(c2ncnc3ccc(-c4cnc(OC)c(NS(=O)(=O)c5ccc(F)cn5)c4)cc23)CC1 ; C=C(F)C(=O)N1CCN(c2ncnc3ccc(-c4cnc(OC)c(NS(=O)(=O)c5ccc(F)cn5)c4)cc23)CC1]

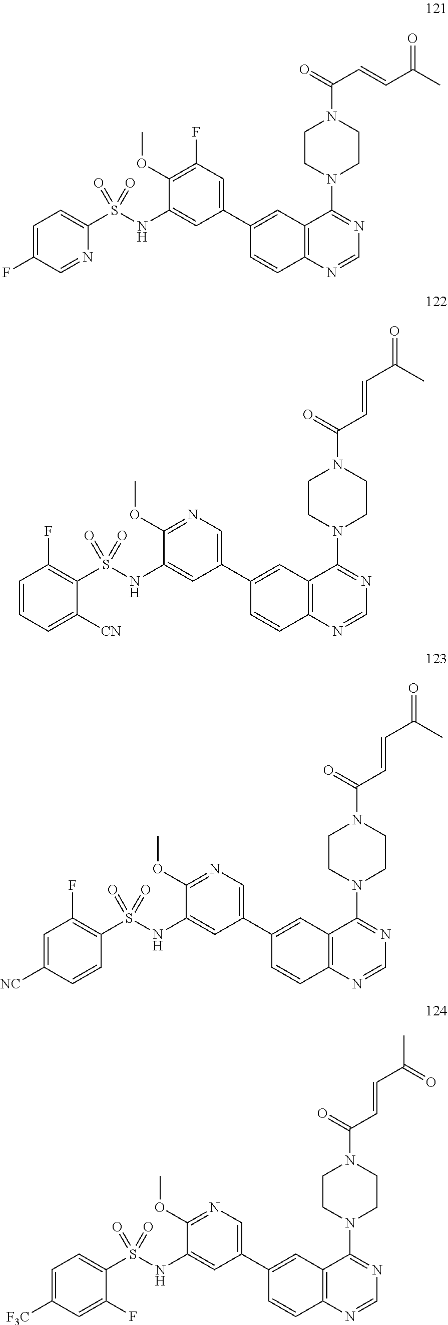 [SMILES: COc1c(F)cc(-c2ccc3ncnc(N4CCN(C(=O)/C=C/C(C)=O)CC4)c3c2)cc1NS(=O)(=O)c1ccc(F)cn1 ; COc1ncc(-c2ccc3ncnc(N4CCN(C(=O)/C=C/C(C)=O)CC4)c3c2)cc1NS(=O)(=O)c1c(F)cccc1C#N ; COc1ncc(-c2ccc3ncnc(N4CCN(C(=O)/C=C/C(C)=O)CC4)c3c2)cc1NS(=O)(=O)c1ccc(C#N)cc1F ; COc1ncc(-c2ccc3ncnc(N4CCN(C(=O)/C=C/C(C)=O)CC4)c3c2)cc1NS(=O)(=O)c1ccc(C(F)(F)F)cc1F]

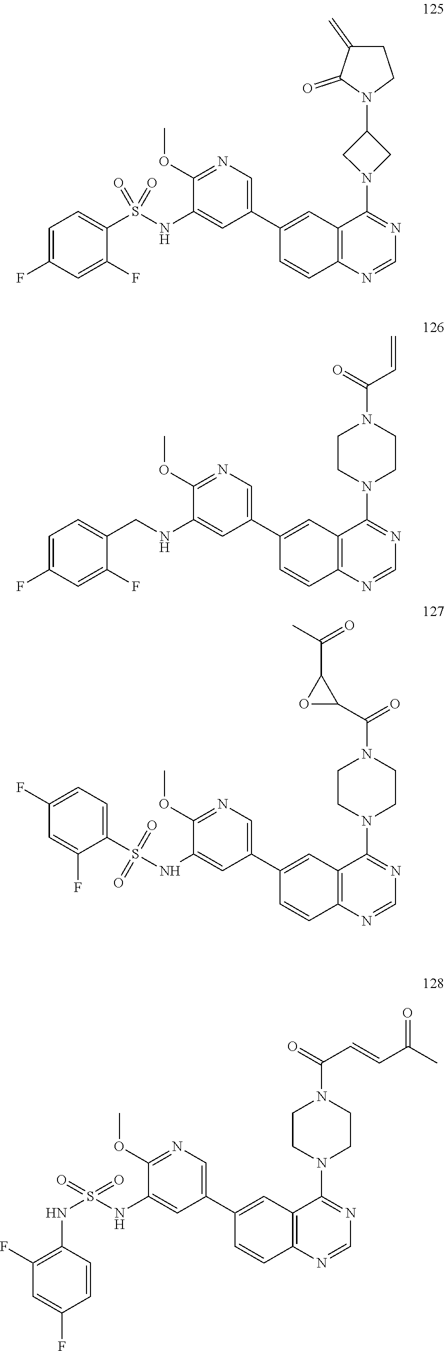

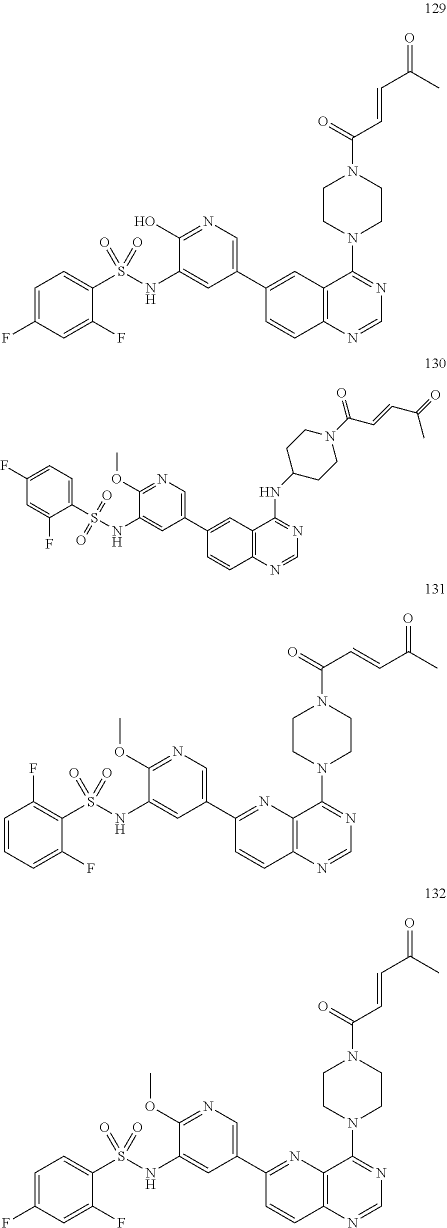 [SMILES: CC(=O)/C=C/C(=O)N1CCN(c2ncnc3ccc(-c4cnc(O)c(NS(=O)(=O)c5ccc(F)cc5F)c4)cc23)CC1 ; COc1ncc(-c2ccc3ncnc(NC4CCN(C(=O)/C=C/C(C)=O)CC4)c3c2)cc1NS(=O)(=O)c1ccc(F)cc1F ; COc1ncc(-c2ccc3ncnc(N4CCN(C(=O)/C=C/C(C)=O)CC4)c3n2)cc1NS(=O)(=O)c1c(F)cccc1F ; COc1ncc(-c2ccc3ncnc(N4CCN(C(=O)/C=C/C(C)=O)CC4)c3n2)cc1NS(=O)(=O)c1ccc(F)cc1F]

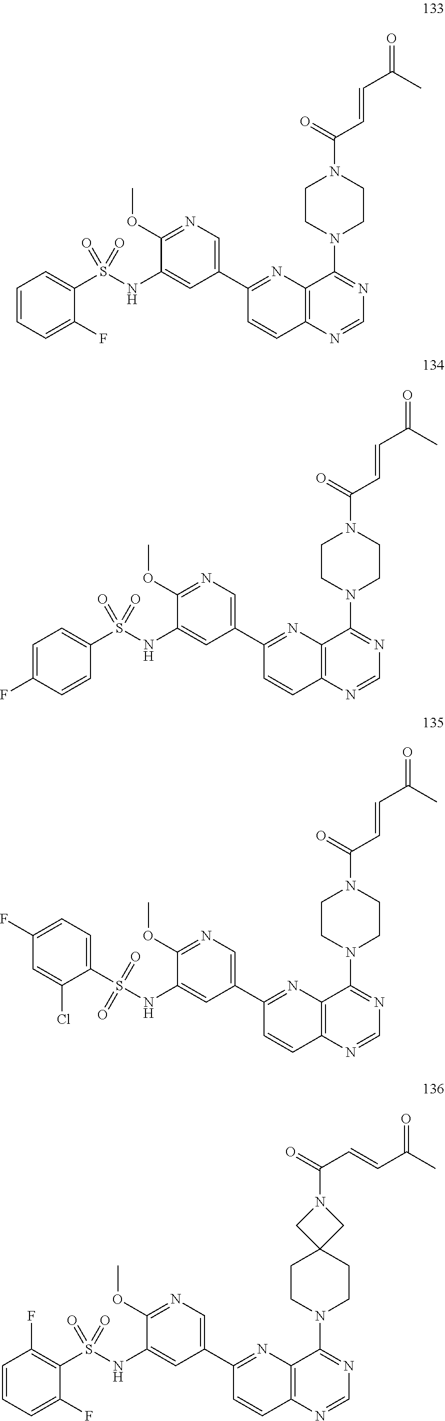 [SMILES: COc1ncc(-c2ccc3ncnc(N4CCN(C(=O)/C=C/C(C)=O)CC4)c3n2)cc1NS(=O)(=O)c1ccccc1F ; COc1ncc(-c2ccc3ncnc(N4CCN(C(=O)/C=C/C(C)=O)CC4)c3n2)cc1NS(=O)(=O)c1ccc(F)cc1 ; COc1ncc(-c2ccc3ncnc(N4CCN(C(=O)/C=C/C(C)=O)CC4)c3n2)cc1NS(=O)(=O)c1ccc(F)cc1Cl ; COc1ncc(-c2ccc3ncnc(N4CCC5(CC4)CN(C(=O)/C=C/C(C)=O)C5)c3n2)cc1NS(=O)(=O)c1c(F)cccc1F]

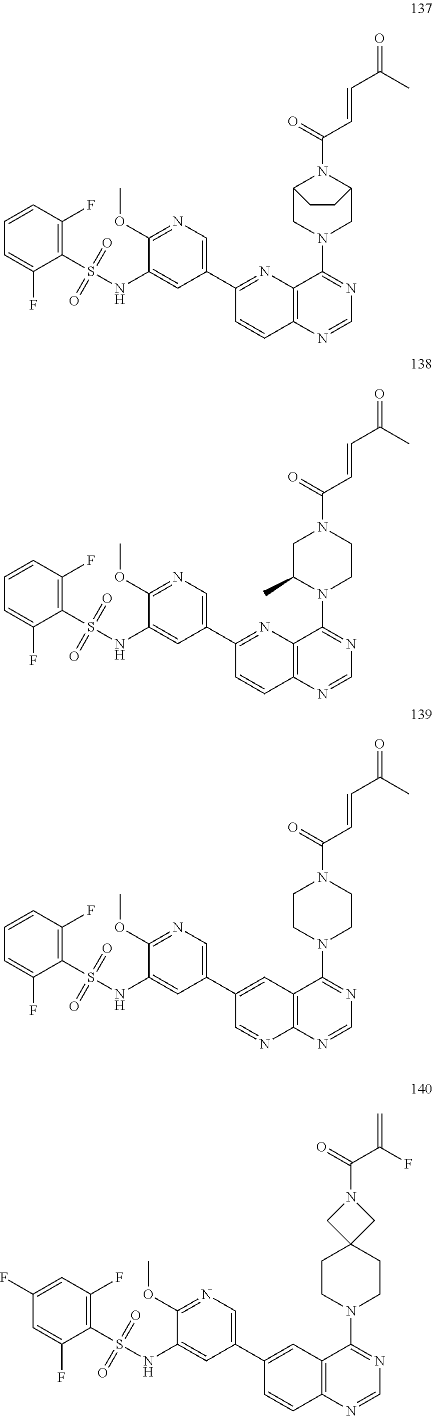 [SMILES: COc1ncc(-c2ccc3ncnc(N4CC5CCC(C4)N5C(=O)/C=C/C(C)=O)c3n2)cc1NS(=O)(=O)c1c(F)cccc1F ; COc1ncc(-c2ccc3ncnc(N4CCN(C(=O)/C=C/C(C)=O)C[C@@H]4C)c3n2)cc1NS(=O)(=O)c1c(F)cccc1F ; COc1ncc(-c2cnc3ncnc(N4CCN(C(=O)/C=C/C(C)=O)CC4)c3c2)cc1NS(=O)(=O)c1c(F)cccc1F ; C=C(F)C(=O)N1CC2(CCN(c3ncnc4ccc(-c5cnc(OC)c(NS(=O)(=O)c6c(F)cc(F)cc6F)c5)cc34)CC2)C1]

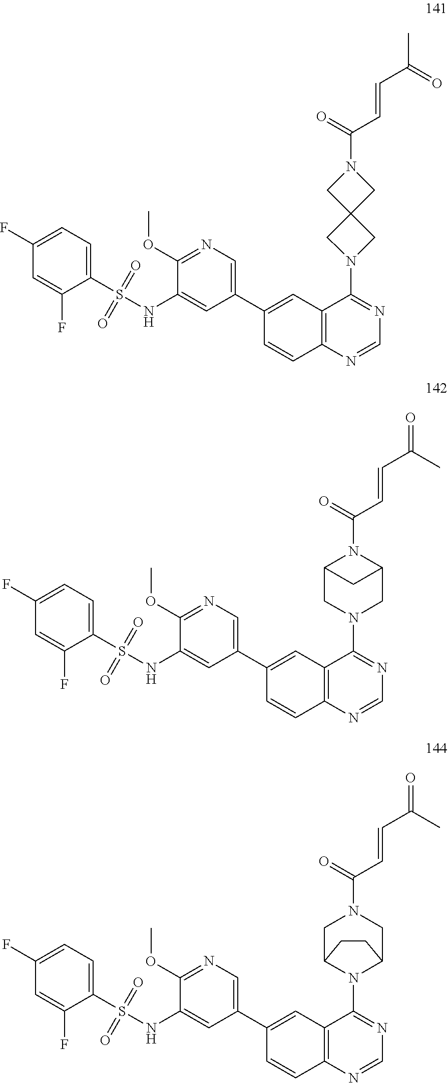 [SMILES: COc1ncc(-c2ccc3ncnc(N4CC5(CN(C(=O)/C=C/C(C)=O)C5)C4)c3c2)cc1NS(=O)(=O)c1ccc(F)cc1F ; COc1ncc(-c2ccc3ncnc(N4CC5CC(C4)N5C(=O)/C=C/C(C)=O)c3c2)cc1NS(=O)(=O)c1ccc(F)cc1F ; COc1ncc(-c2ccc3ncnc(N4C5CCC4CN(C(=O)/C=C/C(C)=O)C5)c3c2)cc1NS(=O)(=O)c1ccc(F)cc1F]

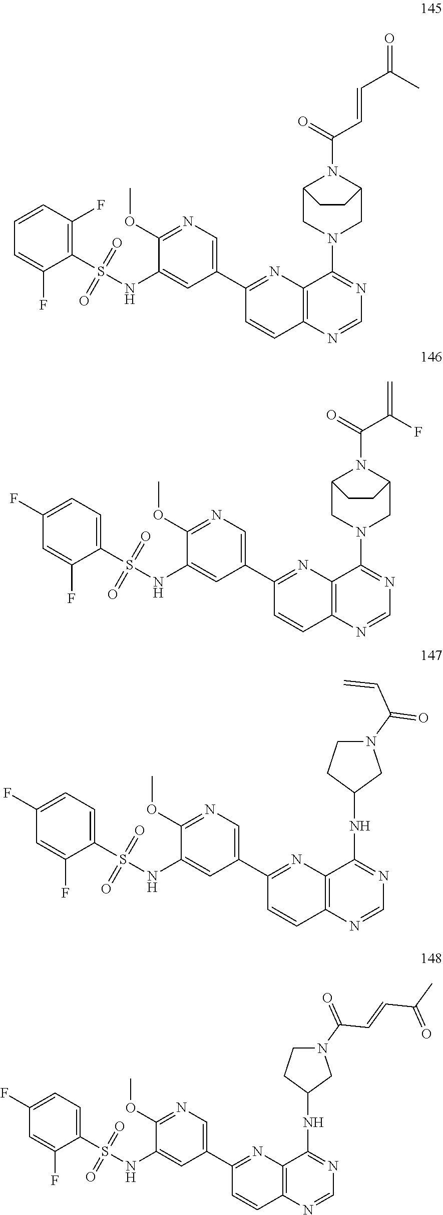 [SMILES: COc1ncc(-c2ccc3ncnc(N4CC5CCC(C4)N5C(=O)/C=C/C(C)=O)c3n2)cc1NS(=O)(=O)c1c(F)cccc1F ; C=C(F)C(=O)N1C2CCC1CN(c1ncnc3ccc(-c4cnc(OC)c(NS(=O)(=O)c5ccc(F)cc5F)c4)nc13)C2 ; C=CC(=O)N1CCC(Nc2ncnc3ccc(-c4cnc(OC)c(NS(=O)(=O)c5ccc(F)cc5F)c4)nc23)C1 ; COc1ncc(-c2ccc3ncnc(NC4CCN(C(=O)/C=C/C(C)=O)C4)c3n2)cc1NS(=O)(=O)c1ccc(F)cc1F]

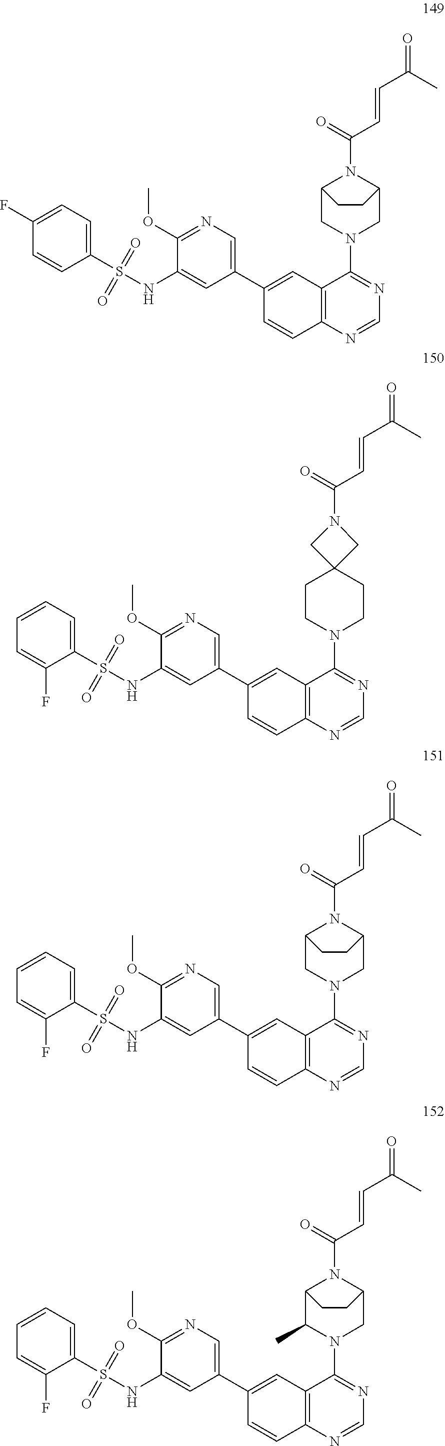 [SMILES: COc1ncc(-c2ccc3ncnc(N4CC5CCC(C4)N5C(=O)/C=C/C(C)=O)c3c2)cc1NS(=O)(=O)c1ccc(F)cc1 ; COc1ncc(-c2ccc3ncnc(N4CCC5(CC4)CN(C(=O)/C=C/C(C)=O)C5)c3c2)cc1NS(=O)(=O)c1ccccc1F ; COc1ncc(-c2ccc3ncnc(N4CC5CCC(C4)N5C(=O)/C=C/C(C)=O)c3c2)cc1NS(=O)(=O)c1ccccc1F ; COc1ncc(-c2ccc3ncnc(N4CC5CCC([C@@H]4C)N5C(=O)/C=C/C(C)=O)c3c2)cc1NS(=O)(=O)c1ccccc1F]

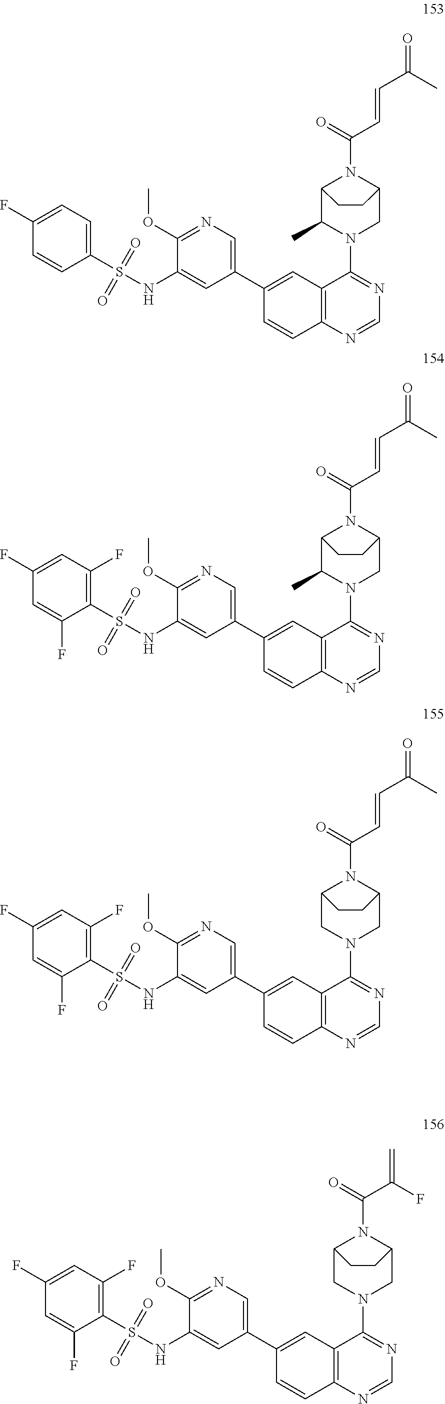 [SMILES: COc1ncc(-c2ccc3ncnc(N4CC5CCC([C@@H]4C)N5C(=O)/C=C/C(C)=O)c3c2)cc1NS(=O)(=O)c1ccc(F)cc1 ; COc1ncc(-c2ccc3ncnc(N4CC5CCC([C@@H]4C)N5C(=O)/C=C/C(C)=O)c3c2)cc1NS(=O)(=O)c1c(F)cc(F)cc1F ; COc1ncc(-c2ccc3ncnc(N4CC5CCC(C4)N5C(=O)/C=C/C(C)=O)c3c2)cc1NS(=O)(=O)c1c(F)cc(F)cc1F ; C=C(F)C(=O)N1C2CCC1CN(c1ncnc3ccc(-c4cnc(OC)c(NS(=O)(=O)c5c(F)cc(F)cc5F)c4)cc13)C2]

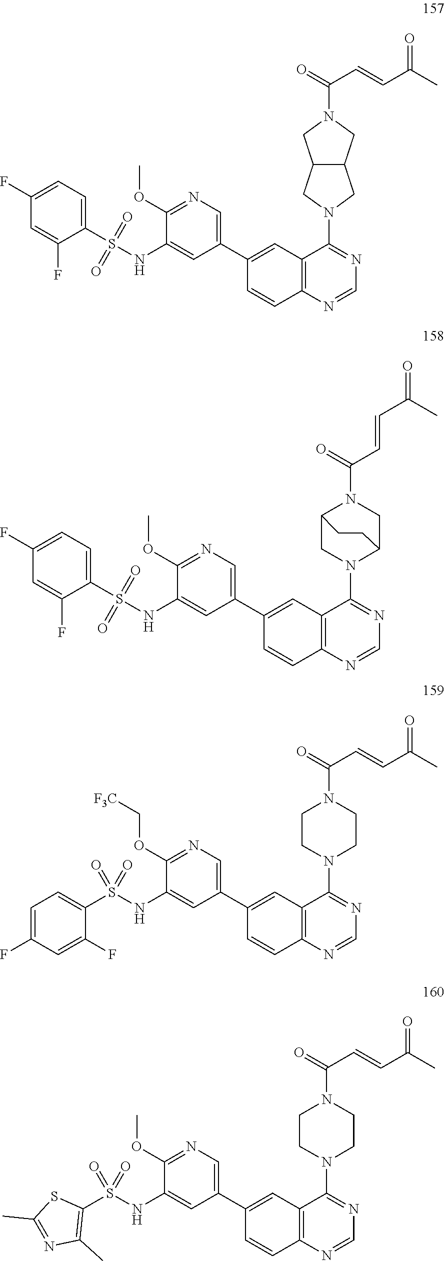

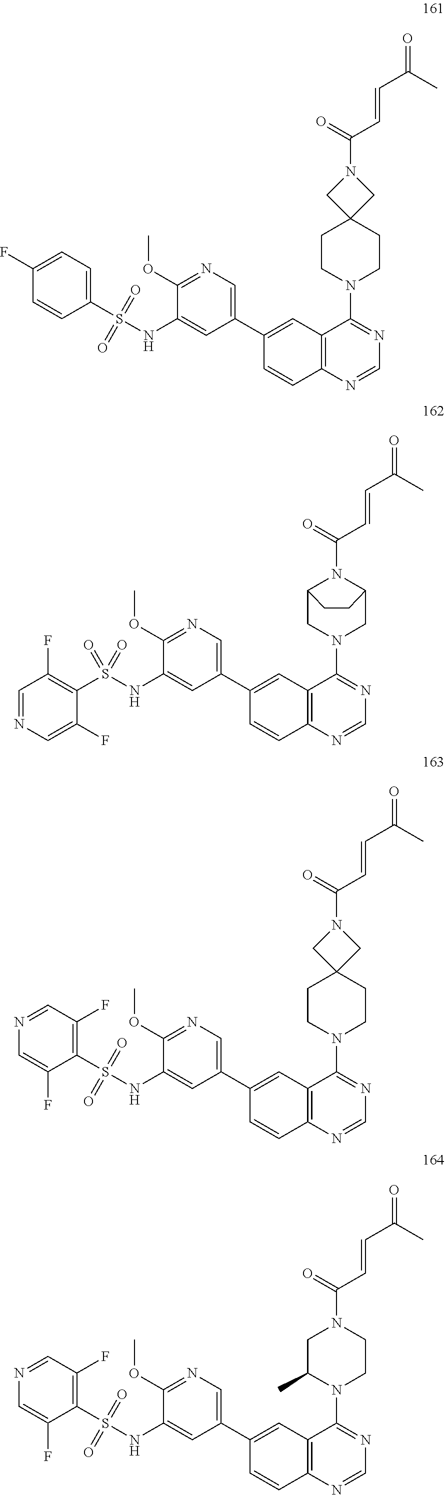 [SMILES: COc1ncc(-c2ccc3ncnc(N4CCC5(CC4)CN(C(=O)/C=C/C(C)=O)C5)c3c2)cc1NS(=O)(=O)c1ccc(F)cc1 ; COc1ncc(-c2ccc3ncnc(N4CC5CCC(C4)N5C(=O)/C=C/C(C)=O)c3c2)cc1NS(=O)(=O)c1c(F)cncc1F ; COc1ncc(-c2ccc3ncnc(N4CCC5(CC4)CN(C(=O)/C=C/C(C)=O)C5)c3c2)cc1NS(=O)(=O)c1c(F)cncc1F ; COc1ncc(-c2ccc3ncnc(N4CCN(C(=O)/C=C/C(C)=O)C[C@@H]4C)c3c2)cc1NS(=O)(=O)c1c(F)cncc1F]

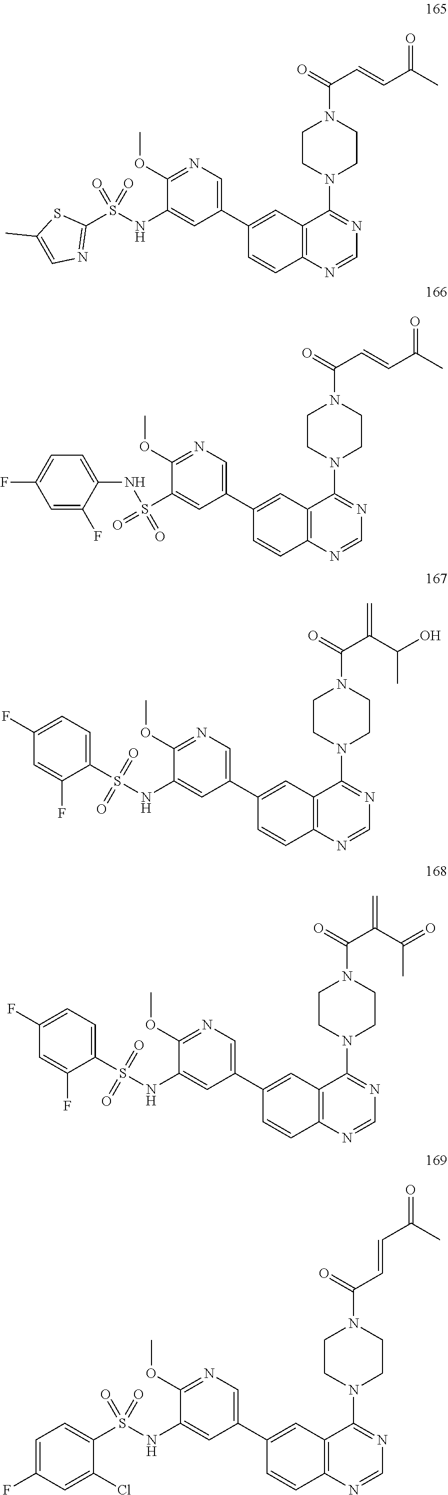 [SMILES: COc1ncc(-c2ccc3ncnc(N4CCN(C(=O)/C=C/C(C)=O)CC4)c3c2)cc1NS(=O)(=O)c1ncc(C)s1 ; COc1ncc(-c2ccc3ncnc(N4CCN(C(=O)/C=C/C(C)=O)CC4)c3c2)cc1S(=O)(=O)Nc1ccc(F)cc1F ; C=C(C(=O)N1CCN(c2ncnc3ccc(-c4cnc(OC)c(NS(=O)(=O)c5ccc(F)cc5F)c4)cc23)CC1)C(C)O ; C=C(C(C)=O)C(=O)N1CCN(c2ncnc3ccc(-c4cnc(OC)c(NS(=O)(=O)c5ccc(F)cc5F)c4)cc23)CC1 ; COc1ncc(-c2ccc3ncnc(N4CCN(C(=O)/C=C/C(C)=O)CC4)c3c2)cc1NS(=O)(=O)c1ccc(F)cc1Cl]

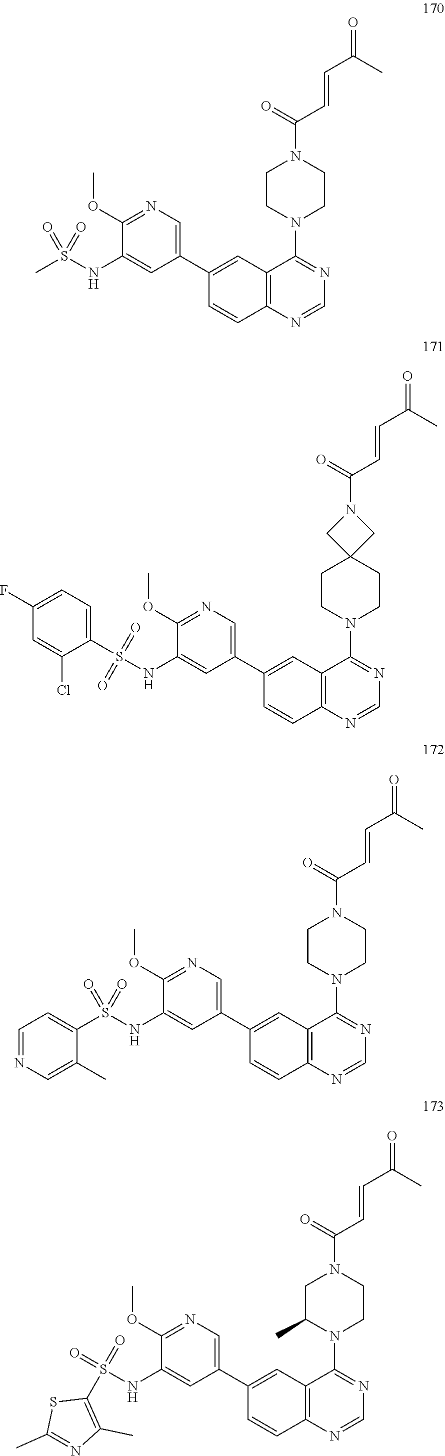

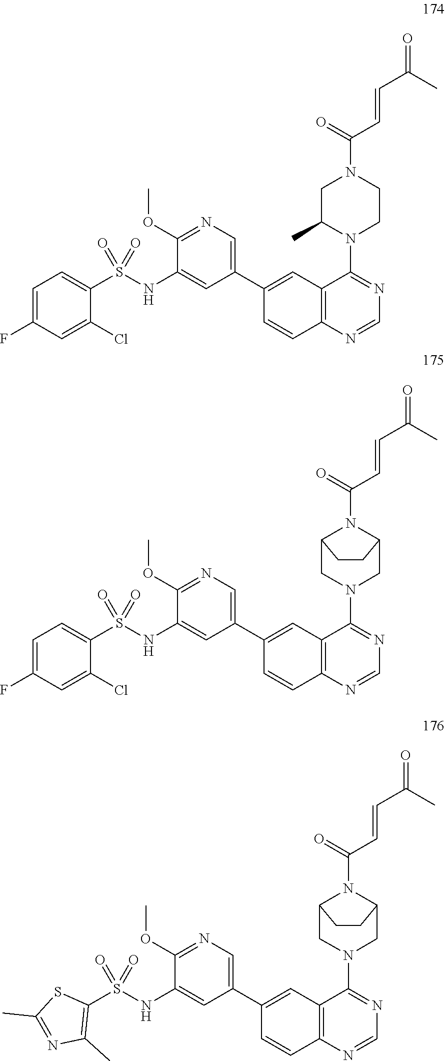

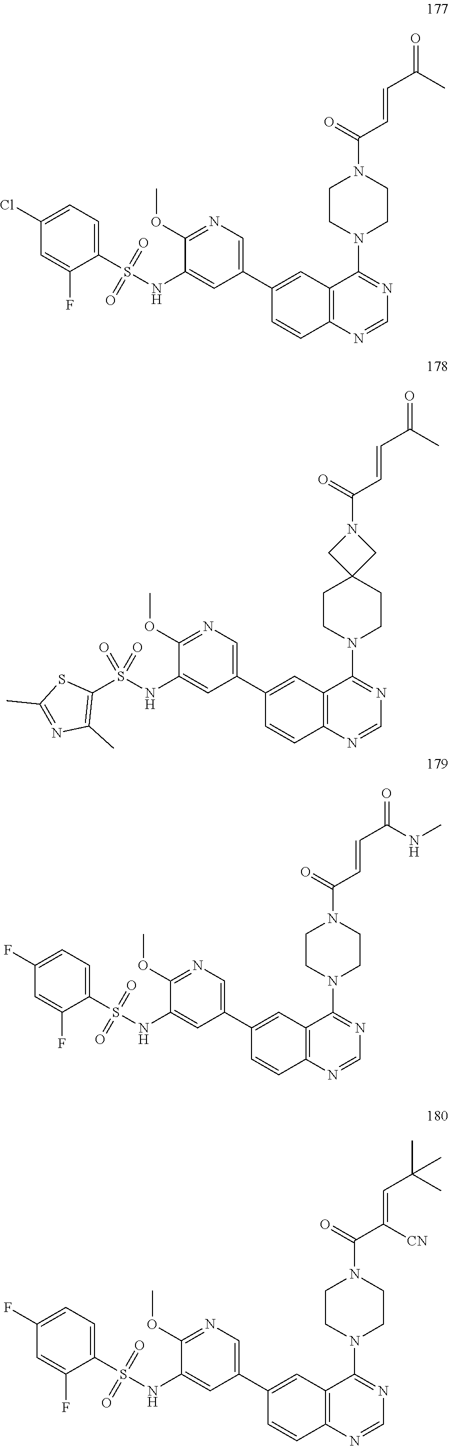

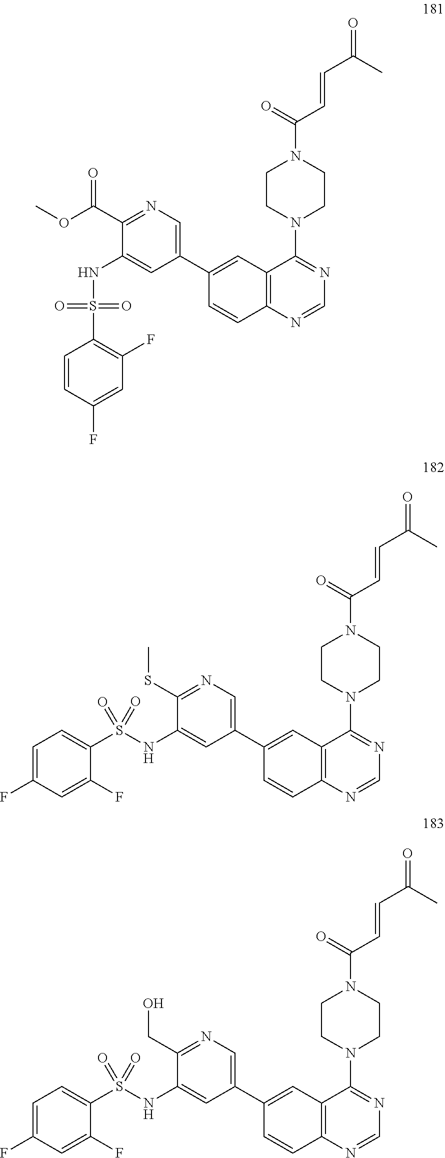 [SMILES: COC(=O)c1ncc(-c2ccc3ncnc(N4CCN(C(=O)/C=C/C(C)=O)CC4)c3c2)cc1NS(=O)(=O)c1ccc(F)cc1F ; CSc1ncc(-c2ccc3ncnc(N4CCN(C(=O)/C=C/C(C)=O)CC4)c3c2)cc1NS(=O)(=O)c1ccc(F)cc1F ; CC(=O)/C=C/C(=O)N1CCN(c2ncnc3ccc(-c4cnc(CO)c(NS(=O)(=O)c5ccc(F)cc5F)c4)cc23)CC1]

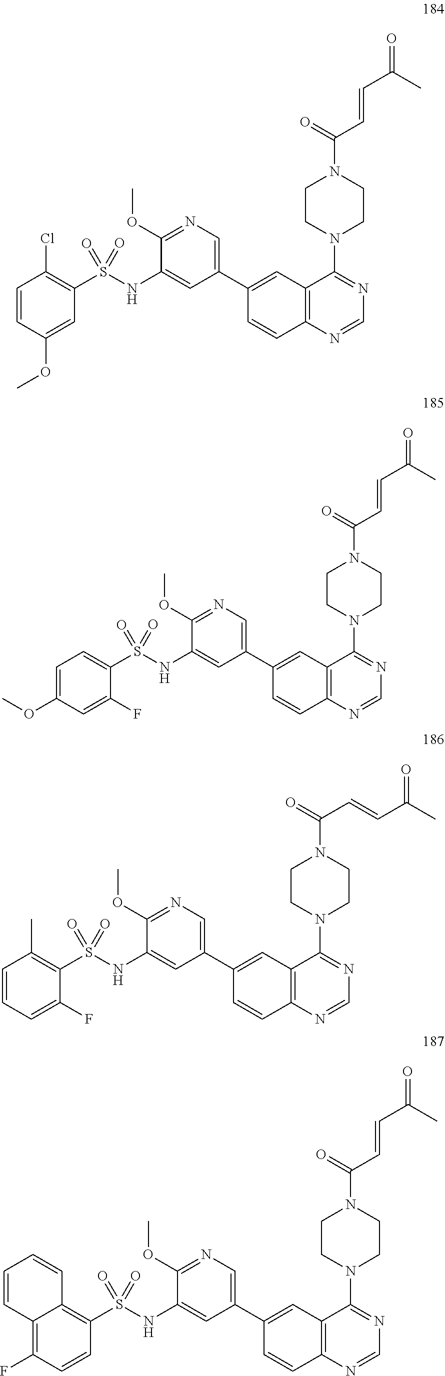

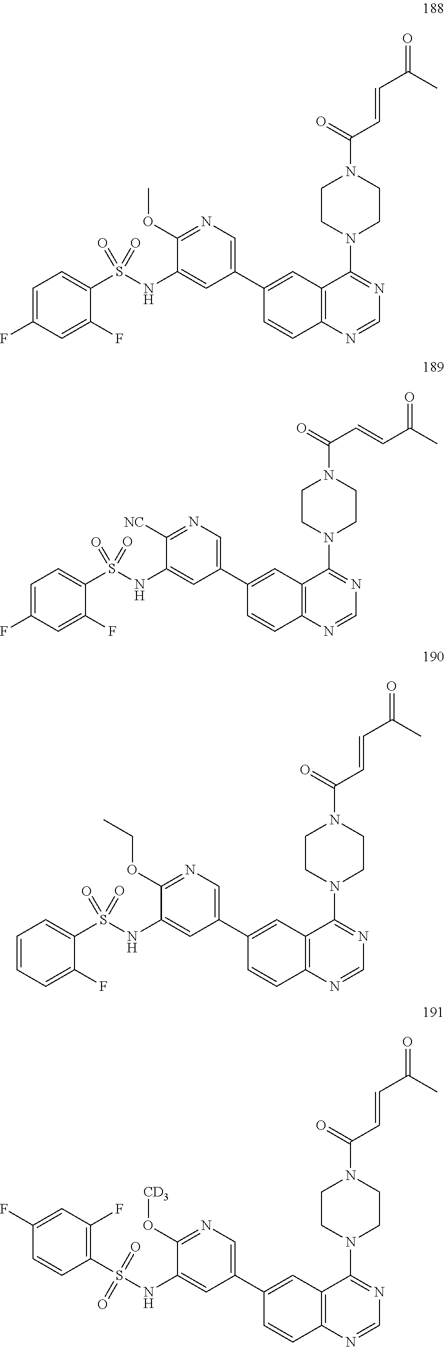 [SMILES: COc1ncc(-c2ccc3ncnc(N4CCN(C(=O)/C=C/C(C)=O)CC4)c3c2)cc1NS(=O)(=O)c1ccc(F)cc1F ; CC(=O)/C=C/C(=O)N1CCN(c2ncnc3ccc(-c4cnc(C#N)c(NS(=O)(=O)c5ccc(F)cc5F)c4)cc23)CC1 ; CCOc1ncc(-c2ccc3ncnc(N4CCN(C(=O)/C=C/C(C)=O)CC4)c3c2)cc1NS(=O)(=O)c1ccccc1F ; [2H]C([2H])([2H])Oc1ncc(-c2ccc3ncnc(N4CCN(C(=O)/C=C/C(C)=O)CC4)c3c2)cc1NS(=O)(=O)c1ccc(F)cc1F]

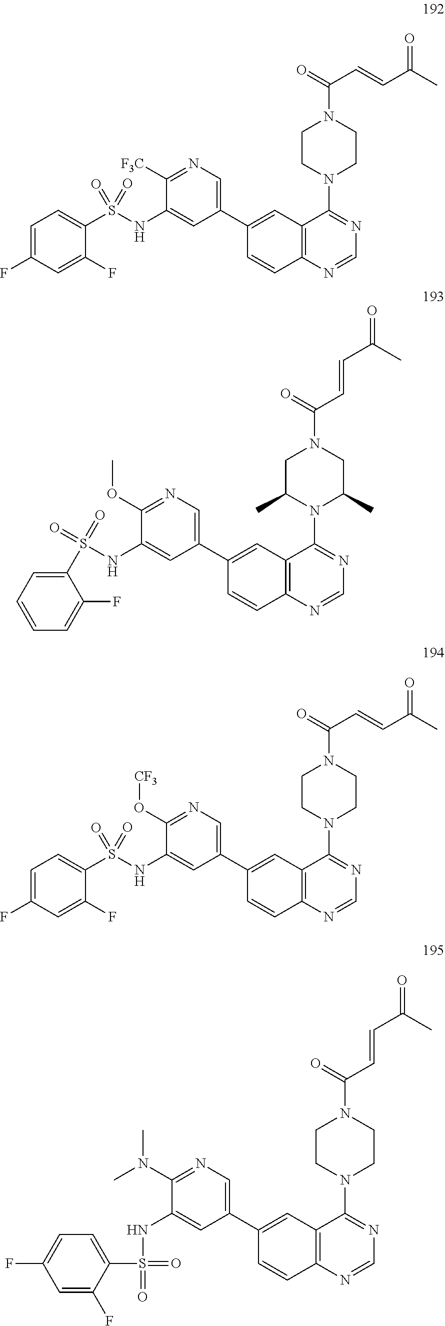 [SMILES: CC(=O)/C=C/C(=O)N1CCN(c2ncnc3ccc(-c4cnc(C(F)(F)F)c(NS(=O)(=O)c5ccc(F)cc5F)c4)cc23)CC1 ; COc1ncc(-c2ccc3ncnc(N4[C@H](C)CN(C(=O)/C=C/C(C)=O)C[C@@H]4C)c3c2)cc1NS(=O)(=O)c1ccccc1F ; CC(=O)/C=C/C(=O)N1CCN(c2ncnc3ccc(-c4cnc(OC(F)(F)F)c(NS(=O)(=O)c5ccc(F)cc5F)c4)cc23)CC1 ; CC(=O)/C=C/C(=O)N1CCN(c2ncnc3ccc(-c4cnc(N(C)C)c(NS(=O)(=O)c5ccc(F)cc5F)c4)cc23)CC1]

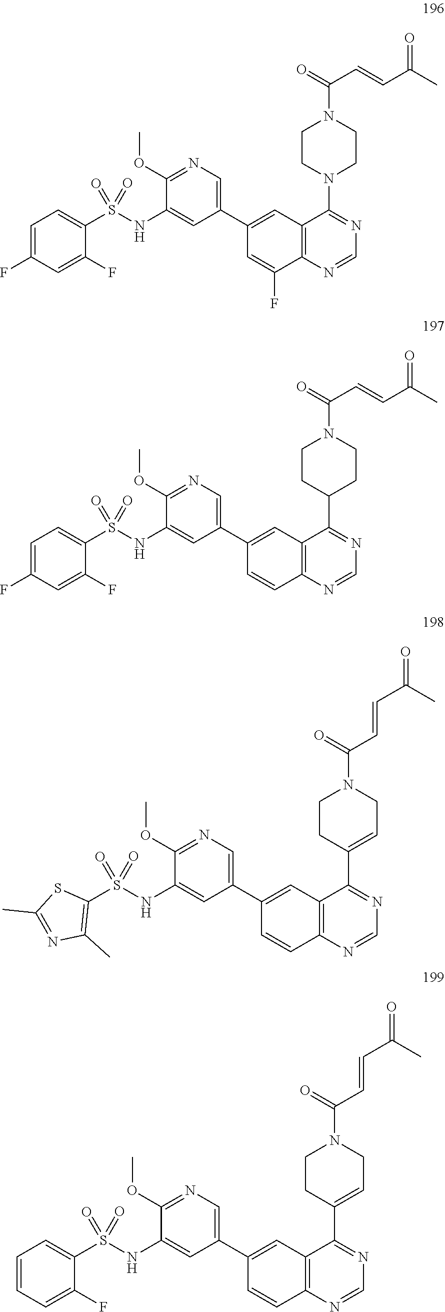

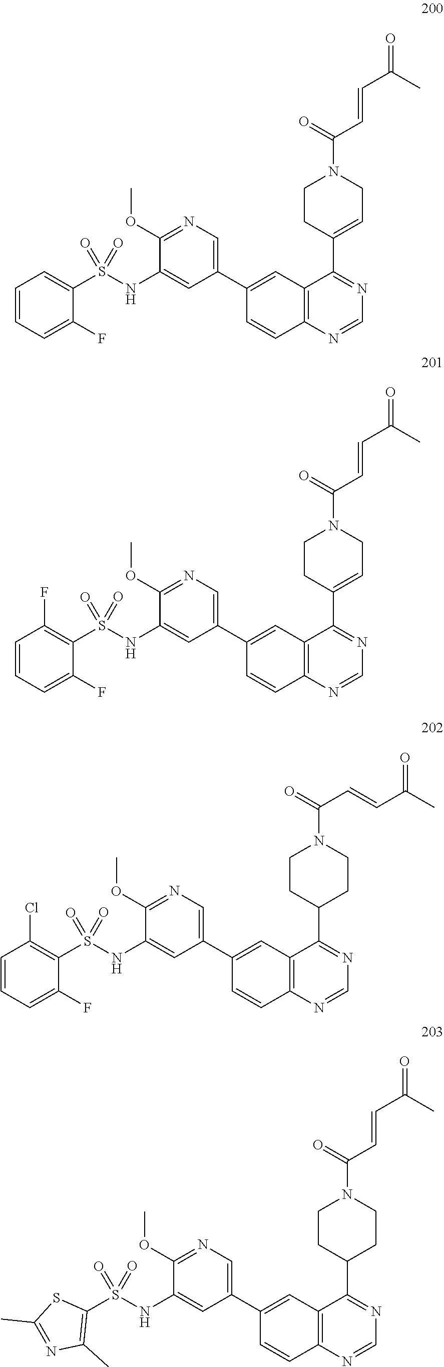

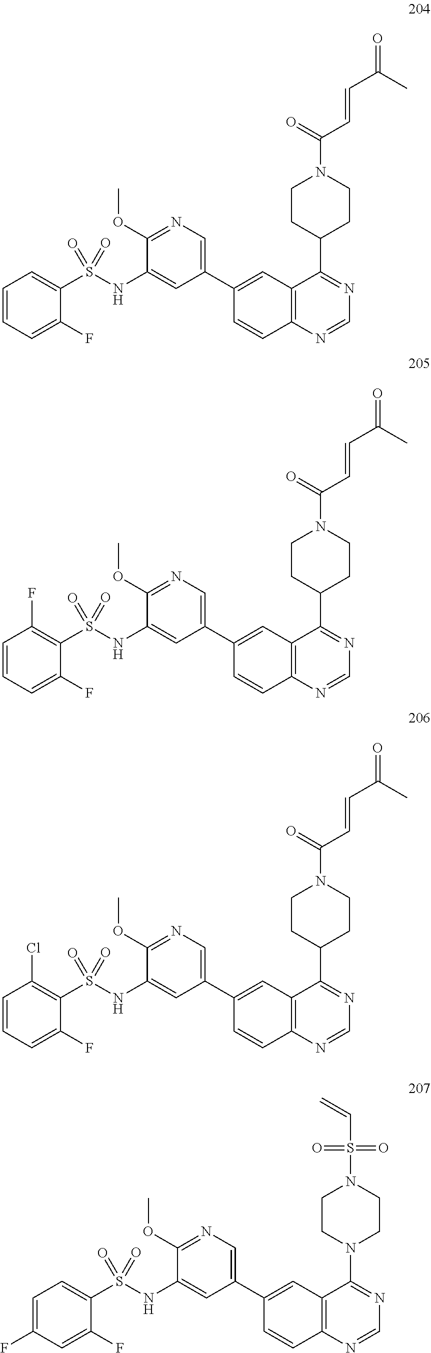 [SMILES: COc1ncc(-c2ccc3ncnc(C4CCN(C(=O)/C=C/C(C)=O)CC4)c3c2)cc1NS(=O)(=O)c1ccccc1F ; COc1ncc(-c2ccc3ncnc(C4CCN(C(=O)/C=C/C(C)=O)CC4)c3c2)cc1NS(=O)(=O)c1c(F)cccc1F ; COc1ncc(-c2ccc3ncnc(C4CCN(C(=O)/C=C/C(C)=O)CC4)c3c2)cc1NS(=O)(=O)c1c(F)cccc1Cl ; C=CS(=O)(=O)N1CCN(c2ncnc3ccc(-c4cnc(OC)c(NS(=O)(=O)c5ccc(F)cc5F)c4)cc23)CC1]

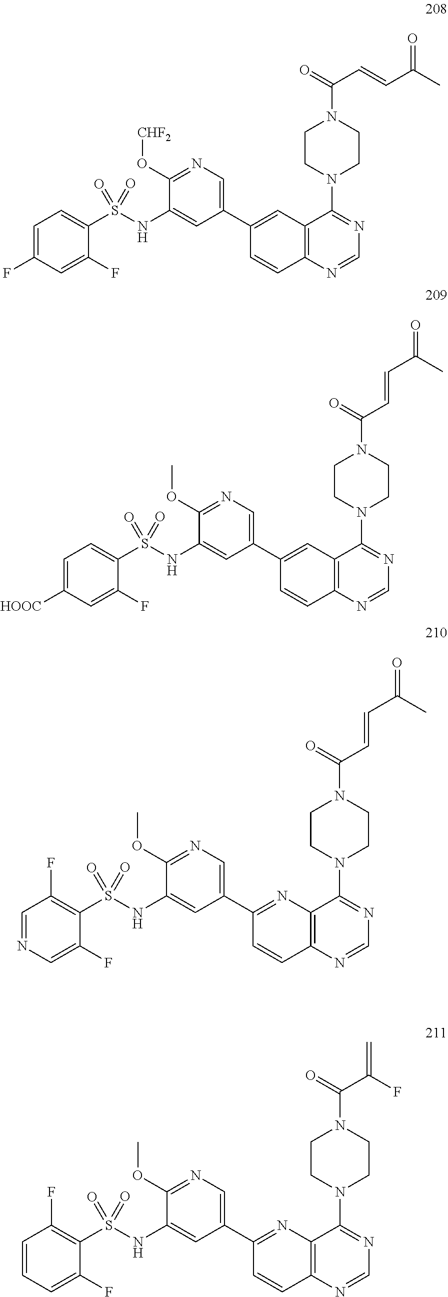 [SMILES: CC(=O)/C=C/C(=O)N1CCN(c2ncnc3ccc(-c4cnc(OC(F)F)c(NS(=O)(=O)c5ccc(F)cc5F)c4)cc23)CC1 ; COc1ncc(-c2ccc3ncnc(N4CCN(C(=O)/C=C/C(C)=O)CC4)c3c2)cc1NS(=O)(=O)c1ccc(C(=O)O)cc1F ; COc1ncc(-c2ccc3ncnc(N4CCN(C(=O)/C=C/C(C)=O)CC4)c3n2)cc1NS(=O)(=O)c1c(F)cncc1F ; C=C(F)C(=O)N1CCN(c2ncnc3ccc(-c4cnc(OC)c(NS(=O)(=O)c5c(F)cccc5F)c4)nc23)CC1]

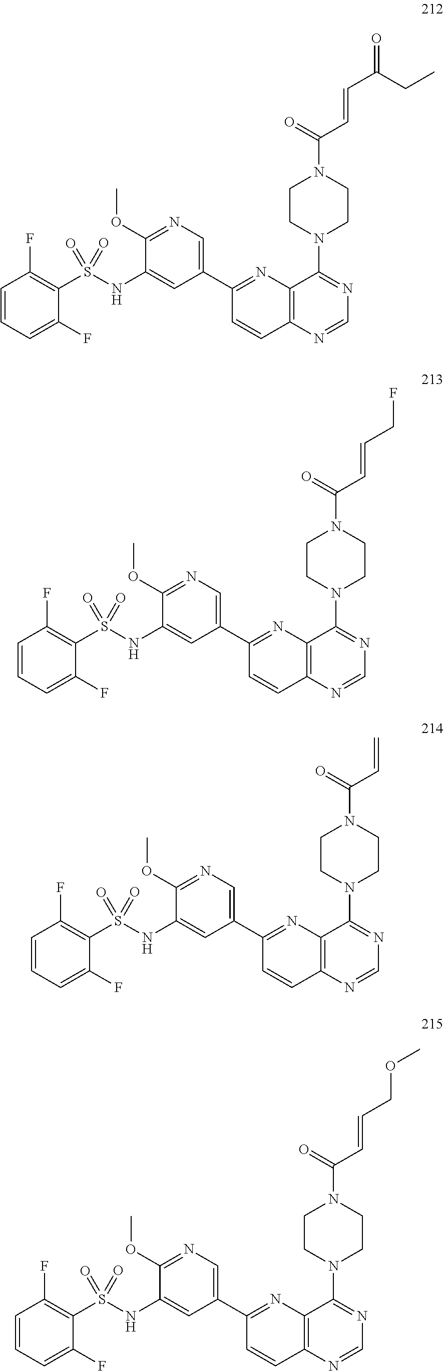 [SMILES: CCC(=O)/C=C/C(=O)N1CCN(c2ncnc3ccc(-c4cnc(OC)c(NS(=O)(=O)c5c(F)cccc5F)c4)nc23)CC1 ; COc1ncc(-c2ccc3ncnc(N4CCN(C(=O)/C=C/CF)CC4)c3n2)cc1NS(=O)(=O)c1c(F)cccc1F ; C=CC(=O)N1CCN(c2ncnc3ccc(-c4cnc(OC)c(NS(=O)(=O)c5c(F)cccc5F)c4)nc23)CC1 ; COC/C=C/C(=O)N1CCN(c2ncnc3ccc(-c4cnc(OC)c(NS(=O)(=O)c5c(F)cccc5F)c4)nc23)CC1]

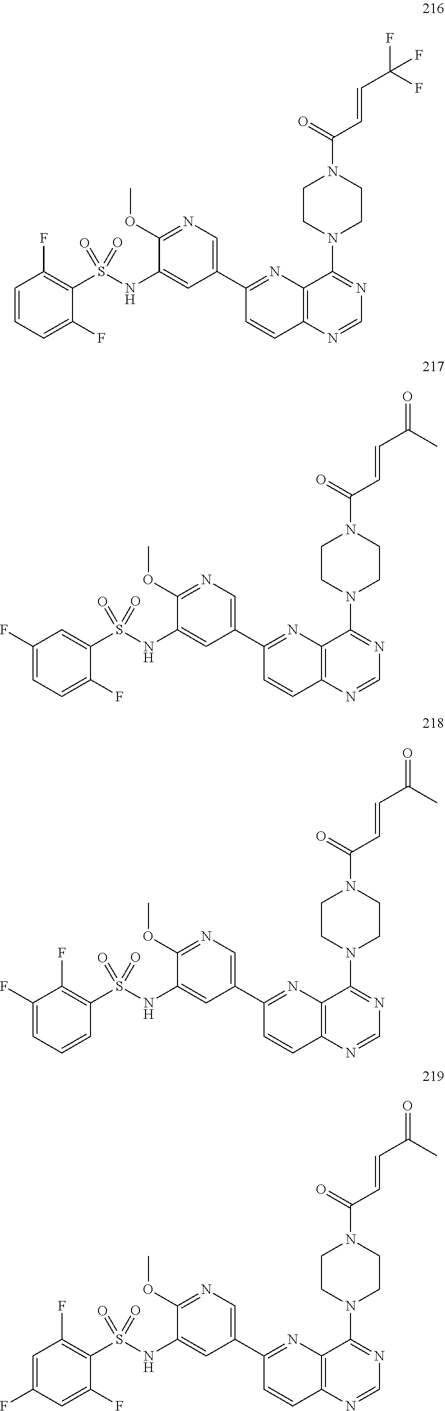 [SMILES: COc1ncc(-c2ccc3ncnc(N4CCN(C(=O)/C=C/C(F)(F)F)CC4)c3n2)cc1NS(=O)(=O)c1c(F)cccc1F ; COc1ncc(-c2ccc3ncnc(N4CCN(C(=O)/C=C/C(C)=O)CC4)c3n2)cc1NS(=O)(=O)c1cc(F)ccc1F ; COc1ncc(-c2ccc3ncnc(N4CCN(C(=O)/C=C/C(C)=O)CC4)c3n2)cc1NS(=O)(=O)c1cccc(F)c1F ; COc1ncc(-c2ccc3ncnc(N4CCN(C(=O)/C=C/C(C)=O)CC4)c3n2)cc1NS(=O)(=O)c1c(F)cc(F)cc1F]

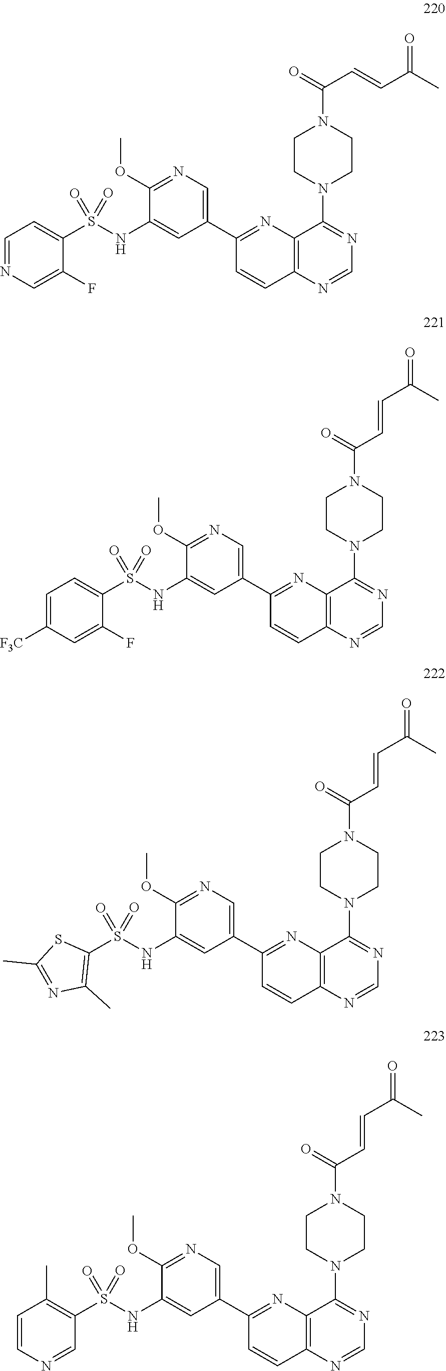

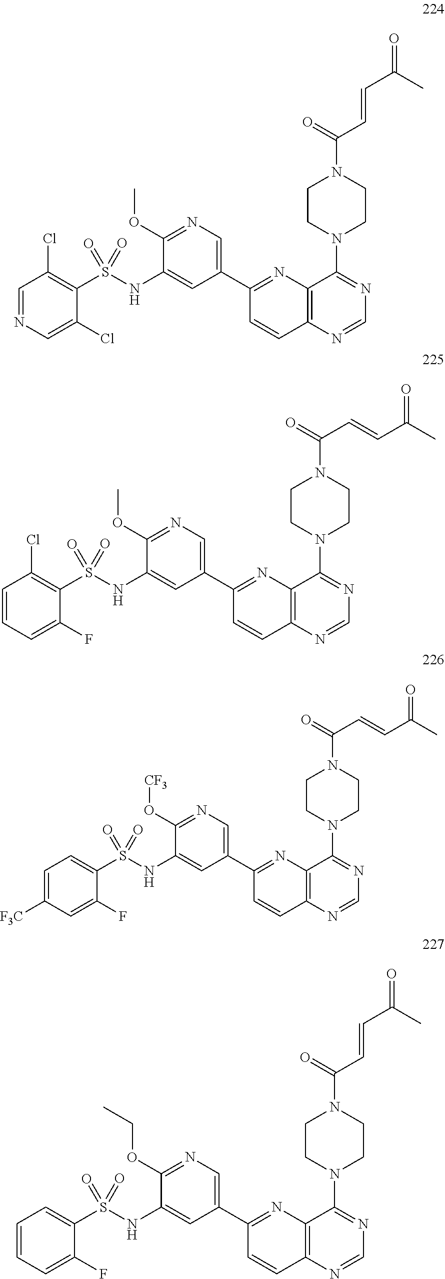 [SMILES: COc1ncc(-c2ccc3ncnc(N4CCN(C(=O)/C=C/C(C)=O)CC4)c3n2)cc1NS(=O)(=O)c1c(Cl)cncc1Cl ; COc1ncc(-c2ccc3ncnc(N4CCN(C(=O)/C=C/C(C)=O)CC4)c3n2)cc1NS(=O)(=O)c1c(F)cccc1Cl ; CC(=O)/C=C/C(=O)N1CCN(c2ncnc3ccc(-c4cnc(OC(F)(F)F)c(NS(=O)(=O)c5ccc(C(F)(F)F)cc5F)c4)nc23)CC1 ; CCOc1ncc(-c2ccc3ncnc(N4CCN(C(=O)/C=C/C(C)=O)CC4)c3n2)cc1NS(=O)(=O)c1ccccc1F]

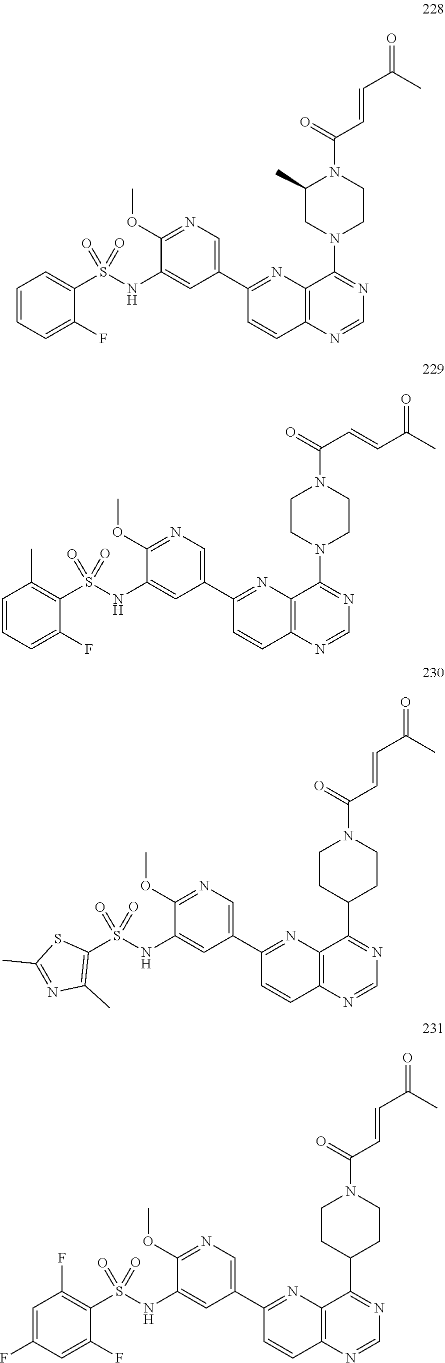

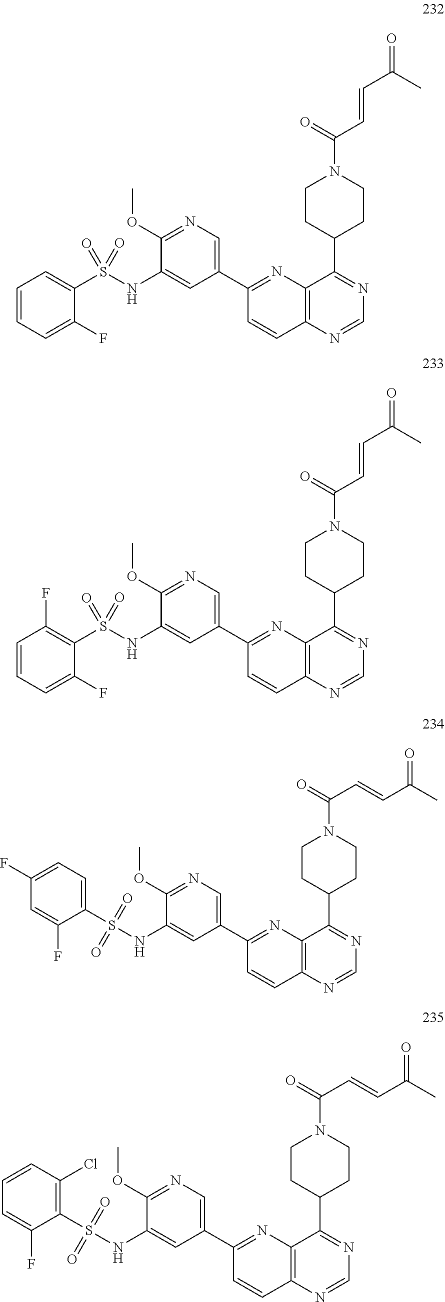 [SMILES: COc1ncc(-c2ccc3ncnc(C4CCN(C(=O)/C=C/C(C)=O)CC4)c3n2)cc1NS(=O)(=O)c1ccccc1F ; COc1ncc(-c2ccc3ncnc(C4CCN(C(=O)/C=C/C(C)=O)CC4)c3n2)cc1NS(=O)(=O)c1c(F)cccc1F ; COc1ncc(-c2ccc3ncnc(C4CCN(C(=O)/C=C/C(C)=O)CC4)c3n2)cc1NS(=O)(=O)c1ccc(F)cc1F ; COc1ncc(-c2ccc3ncnc(C4CCN(C(=O)/C=C/C(C)=O)CC4)c3n2)cc1NS(=O)(=O)c1c(F)cccc1Cl]

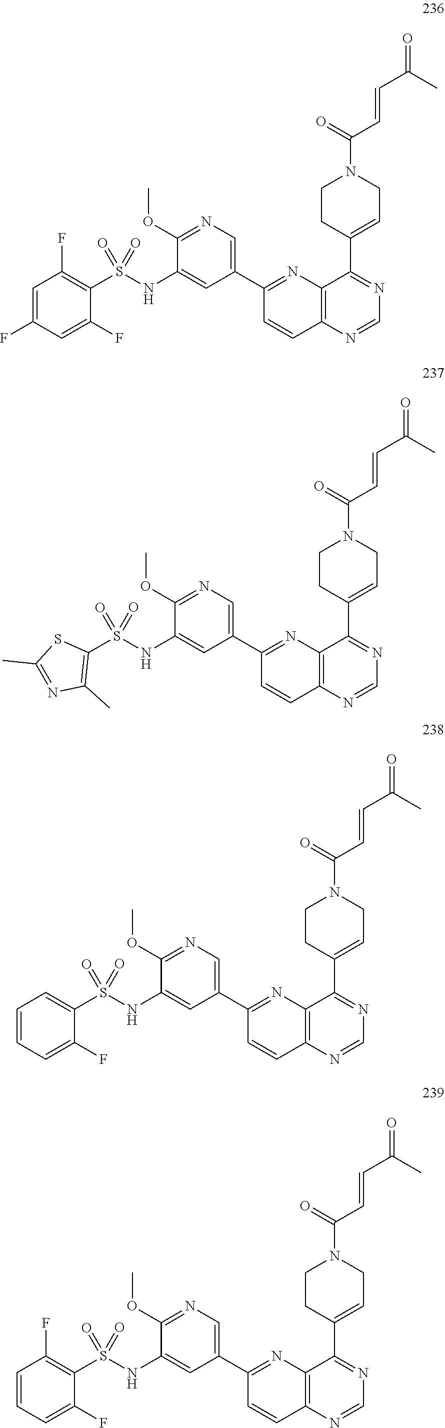

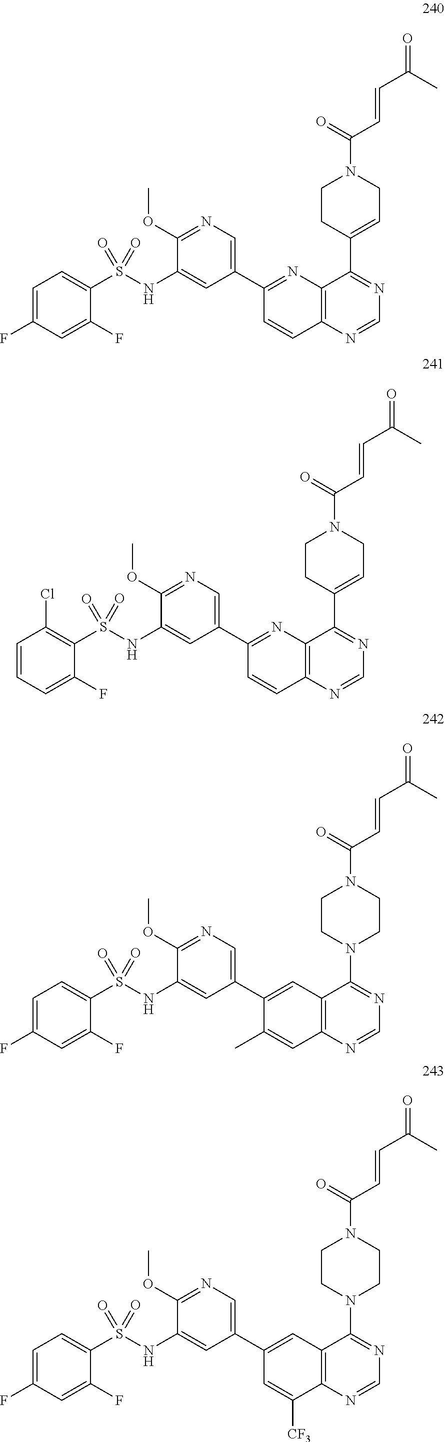 [SMILES: COc1ncc(-c2ccc3ncnc(C4=CCN(C(=O)/C=C/C(C)=O)CC4)c3n2)cc1NS(=O)(=O)c1ccc(F)cc1F ; COc1ncc(-c2ccc3ncnc(C4=CCN(C(=O)/C=C/C(C)=O)CC4)c3n2)cc1NS(=O)(=O)c1c(F)cccc1Cl ; COc1ncc(-c2cc3c(N4CCN(C(=O)/C=C/C(C)=O)CC4)ncnc3cc2C)cc1NS(=O)(=O)c1ccc(F)cc1F ; COc1ncc(-c2cc(C(F)(F)F)c3ncnc(N4CCN(C(=O)/C=C/C(C)=O)CC4)c3c2)cc1NS(=O)(=O)c1ccc(F)cc1F]

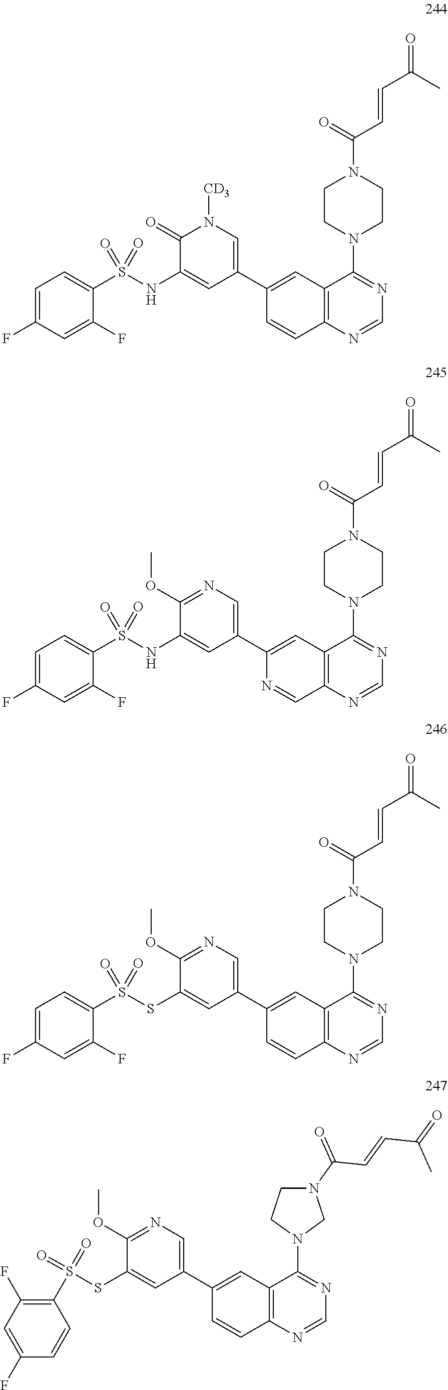 [SMILES: [2H]C([2H])([2H])n1cc(-c2ccc3ncnc(N4CCN(C(=O)/C=C/C(C)=O)CC4)c3c2)cc(NS(=O)(=O)c2ccc(F)cc2F)c1=O ; COc1ncc(-c2cc3c(N4CCN(C(=O)/C=C/C(C)=O)CC4)ncnc3cn2)cc1NS(=O)(=O)c1ccc(F)cc1F ; COc1ncc(-c2ccc3ncnc(N4CCN(C(=O)/C=C/C(C)=O)CC4)c3c2)cc1SS(=O)(=O)c1ccc(F)cc1F ; COc1ncc(-c2ccc3ncnc(N4CCN(C(=O)/C=C/C(C)=O)C4)c3c2)cc1SS(=O)(=O)c1ccc(F)cc1F]

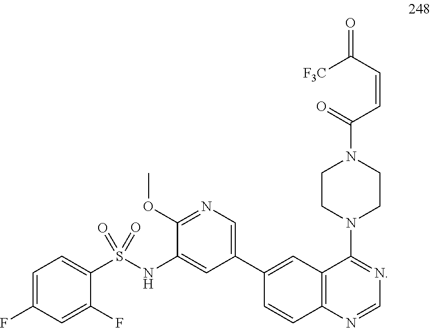 [SMILES: COc1ncc(-c2ccc3ncnc(N4CCN(C(=O)/C=C\C(=O)C(F)(F)F)CC4)c3c2)cc1NS(=O)(=O)c1ccc(F)cc1F]

37. A pharmaceutical composition, wherein the active ingredient of the pharmaceutical composition is selected from one or a combination of two or more of the compound of claim 1, or a tautomer, meso isomer, racemate, enantiomer, diastereomer or mixture thereof, or a pharmaceutically acceptable hydrate, solvate or salt thereof.

38. (canceled)

39. A method of treating a disease mediated by KRAS and/or PI3K, comprising administering to a subject in need thereof a therapeutically effective amount of the compound of claim 1, or a tautomer, meso isomer, racemate, enantiomer, diastereomer or mixture thereof, or a pharmaceutically acceptable hydrate, solvate or salt thereof or pharmaceutical composition, wherein the active ingredient of the pharmaceutical composition is selected from one or a combination of two or more of the compound of claim 1, or a tautomer, meso isomer, racemate, enantiomer, diastereomer or mixture thereof, or a pharmaceutically acceptable hydrate, solvate or salt thereof;
further, wherein the disease mediated by KRAS and/or PI3K comprises a disease mediated by one or more of KRAS G12C, PI3Kα, and PI3Kδ;
preferably, wherein the disease is cancer or an autoimmune disease;
preferably, wherein the cancer is selected from: non-small cell lung cancer, lung cancer, pancreatic cancer, ovarian cancer, bladder cancer, prostate cancer, chronic myelogenous leukemia, colorectal cancer, brain cancer, liver cancer, kidney cancer, stomach cancer, breast cancer, triple negative breast cancer, skin cancer, melanin Cancer, head and neck cancer, bone cancer, cervical cancer, pelvic cancer, vaginal cancer, oral cancer, lymphoma, blood cancer, esophageal cancer, urethra cancer, or nasal cavity cancer.

40. A method of treating a disease that is resistant to anticancer agents, comprising administering to a subject in need thereof a therapeutically effective amount of the compound of claim 1, or a tautomer, meso isomer, racemate, enantiomer, diastereomer or mixture thereof, or a pharmaceutically acceptable hydrate, solvate or salt thereof or a pharmaceutical composition, wherein the active ingredient of the pharmaceutical composition is selected from one or a combination of two or more of the compound of claim 1, or a tautomer, meso isomer, racemate, enantiomer, diastereomer or mixture thereof, or a pharmaceutically acceptable hydrate, solvate or salt thereof;
further, wherein the anticancer agent is selected from RAS G12C inhibitors, KRAS G12V inhibitors, KRAS G12D inhibitors, KRAS G12S inhibitors, preferably KRAS G12C inhibitors;
further, wherein the KRAS G12C inhibitors is selected from AMG-510, MRTX-849, preferably AMG-510.

41. (canceled)

42. (canceled)

43. A pharmaceutical composition, wherein the active ingredient of the pharmaceutical composition is selected from one or a combination of two or more of the compound of claim 16, or a tautomer, meso isomer, racemate, enantiomer, diastereomer or mixture thereof, or a pharmaceutically acceptable hydrate, solvate or salt thereof.

44. A pharmaceutical composition, wherein the active ingredient of the pharmaceutical composition is selected from one or a combination of two or more of the compound of claim 36, or a tautomer, meso isomer, racemate, enantiomer, diastereomer or mixture thereof, or a pharmaceutically acceptable hydrate, solvate or salt thereof.

45. A method of treating a disease mediated by KRAS and/or PI3K, comprising administering to a subject in need thereof a therapeutically effective amount of the compound of claim 16, or a tautomer, meso isomer, racemate, enantiomer, diastereomer or mixture thereof, or a pharmaceutically acceptable hydrate, solvate or salt thereof or a pharmaceutical composition, wherein the active ingredient of the pharmaceutical composition is selected from one or a combination of two or more of the compound of claim 16, or a tautomer, meso isomer, racemate, enantiomer, diastereomer or mixture thereof, or a pharmaceutically acceptable hydrate, solvate or salt thereof:
further, wherein the disease mediated by KRAS and/or PI3K comprises a disease mediated by one or more of KRAS G12C, PI3Kα, and PI3Kδ;
preferably, wherein the disease is cancer or an autoimmune disease;
preferably, wherein the cancer is selected from: non-small cell lung cancer, lung cancer, pancreatic cancer, ovarian cancer, bladder cancer, prostate cancer, chronic myelogenous leukemia, colorectal cancer, brain cancer, liver cancer, kidney cancer, stomach cancer, breast cancer, triple negative breast cancer, skin cancer, melanin Cancer, head and neck cancer, bone cancer, cervical cancer, pelvic cancer, vaginal cancer, oral cancer, lymphoma, blood cancer, esophageal cancer, urethra cancer or nasal cavity cancer.

46. A method of treating a disease mediated by KRAS and/or PI3K, comprising administering to a subject in need thereof a therapeutically effective amount of the compound of claim 36, or a tautomer, meso isomer, racemate, enantiomer, diastereomer or mixture thereof, or a pharmaceutically acceptable hydrate, solvate or salt thereof or a pharmaceutical composition, wherein the active ingredient of the pharmaceutical composition is selected from one or a combination of two or more of the compound of claim 36, or a tautomer, meso isomer, racemate, enantiomer, diastereomer or mixture thereof, or a pharmaceutically acceptable hydrate, solvate or salt thereof;
further, wherein the disease mediated by KRAS and/or PI3K comprises a disease mediated by one or more of KRAS G12C, PI3Kα, and PI3Kδ;
preferably, wherein the disease is cancer or an autoimmune disease;
preferably, wherein the cancer is selected from: non-small cell lung cancer, lung cancer, pancreatic cancer, ovarian cancer, bladder cancer, prostate cancer, chronic myelogenous leukemia, colorectal cancer, brain cancer, liver cancer, kidney cancer, stomach cancer, breast cancer, triple negative breast cancer, skin cancer, melanin Cancer, head and neck cancer, bone cancer, cervical cancer, pelvic cancer, vaginal cancer, oral cancer, lymphoma, blood cancer, esophageal cancer, urethra cancer or nasal cavity cancer.

47. A method of treating a disease that is resistant to anticancer agents, comprising administering to a subject in need thereof a therapeutically effective amount of the compound of claim 16, or a tautomer, meso isomer, racemate, enantiomer, diastereomer or mixture thereof, or a pharmaceutically acceptable hydrate, solvate or salt thereof or a pharmaceutical composition, wherein the active ingredient of the pharmaceutical composition is selected from one or a combination of two or more of the compound of claim 16, or a tautomer, meso isomer, racemate, enantiomer, diastereomer or mixture thereof, or a pharmaceutically acceptable hydrate, solvate or salt thereof,
further, wherein the anticancer agent is selected from KRAS G12C inhibitors, KRAS G12V inhibitors, KRAS G12D inhibitors, KRAS G12S inhibitors, preferably KRAS G12C inhibitors;
further, wherein the KRAS G12C inhibitors is selected from AMG-510, MRTX-849, preferably AMG-510.

48. A method of treating a disease that is resistant to anticancer agents, comprising administering to a subject in need thereof a therapeutically effective amount of the compound of claim 36, or a tautomer, meso isomer, racemate, enantiomer, diastereomer or mixture thereof, or a pharmaceutically acceptable hydrate, solvate or salt thereof or a pharmaceutical composition, wherein the active ingredient of the pharmaceutical composition is selected from one or a combination of two or more of the compound of claim 36, or a tautomer, meso isomer, racemate, enantiomer, diastereomer or mixture thereof, or a pharmaceutically acceptable hydrate, solvate or salt thereof,
further, wherein the anticancer agent is selected from KRAS G12C inhibitors, KRAS G12V inhibitors, KRAS G12D inhibitors, KRAS G12S inhibitors, preferably KRAS G12C inhibitors;
further, wherein the KRAS G12C inhibitors is selected from AMG-510, MRTX-849, preferably AMG-510.
